(19) United States
(12) Patent Application Publication
(10) Pub. No.: US 2024/0276874 A1
(43) Pub. Date: Aug. 15, 2024

(54) LIGHT-EMITTING DEVICE INCLUDING AMINE-CONTAINING COMPOUND, ELECTRONIC APPARATUS INCLUDING LIGHT-EMITTING DEVICE, AND AMINE-CONTAINING COMPOUND

(71) Applicant: Samsung Display Co., Ltd., Yongin-si (KR)

(72) Inventors: Hankyu Pak, Yongin-si (KR); Minji Kim, Yongin-si (KR); Hyunbin Park, Yongin-si (KR); Byeongwook Yoo, Yongin-si (KR); Jeongmin Lee, Yongin-si (KR); Sohee Jo, Yongin-si (KR)

(21) Appl. No.: 18/514,709

(22) Filed: Nov. 20, 2023

(30) Foreign Application Priority Data
Jan. 5, 2023 (KR) 10-2023-0001951

(51) Int. Cl. H10K 85/60; C07C 211/61

(52) U.S. Cl. CPC H10K 85/633; C07C 211/61; H10K 50/15

(57) ABSTRACT

A light-emitting device including a first electrode, a second electrode facing the first electrode, and an interlayer between the first electrode and the second electrode and including an amine-containing compound represented by Formula 1, an electronic apparatus including the light-emitting device, and the amine-containing compound represented by Formula 1 are provided.

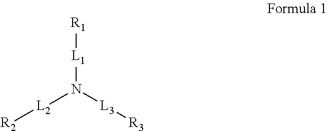

### CROSS-REFERENCE TO RELATED APPLICATION

This application claims priority to and the benefit of Korean Patent Application No. 10-2023-0001951, filed on Jan. 5, 2023, in the Korean Intellectual Property Office, the content of which is incorporated by reference herein in its entirety.

### BACKGROUND

### 1. Field

One or more embodiments of the present disclosure relate to a light-emitting device including an amine-containing compound, an electronic apparatus including the light-emitting device, and the amine-containing compound.

### 2. Description of the Related Art

Light-emitting devices (for example, organic light-emitting devices) are self-emissive devices that have relatively wide viewing angles, high contrast ratios, short response times, and excellent or suitable characteristics in terms of brightness, driving voltage, and response speed.

Light-emitting devices may include a first electrode on a substrate, and a hole transport region, an emission layer, an electron transport region, and a second electrode sequentially stacked on the first electrode. Holes provided from the first electrode may move toward the emission layer through the hole transport region, and electrons provided from the second electrode may move toward the emission layer through the electron transport region. Carriers, such as holes and electrons, recombine in the emission layer to produce excitons. These excitons transition and decay from an excited state to a ground state to thus generate light.

### SUMMARY

One or more aspects of embodiments of the present disclosure are directed toward a light-emitting device including an amine-containing compound, an electronic apparatus including the light-emitting device, and the amine-containing compound.

Additional aspects will be set forth in part in the description which follows and, in part, will be apparent from the description, or may be learned by practice of the presented embodiments of the disclosure.

According to one or more embodiments of the present disclosure, a light-emitting device may include:
a first electrode;
a second electrode facing the first electrode;
an interlayer between the first electrode and the second electrode and including an emission layer; and
an amine-containing compound represented by Formula 1:

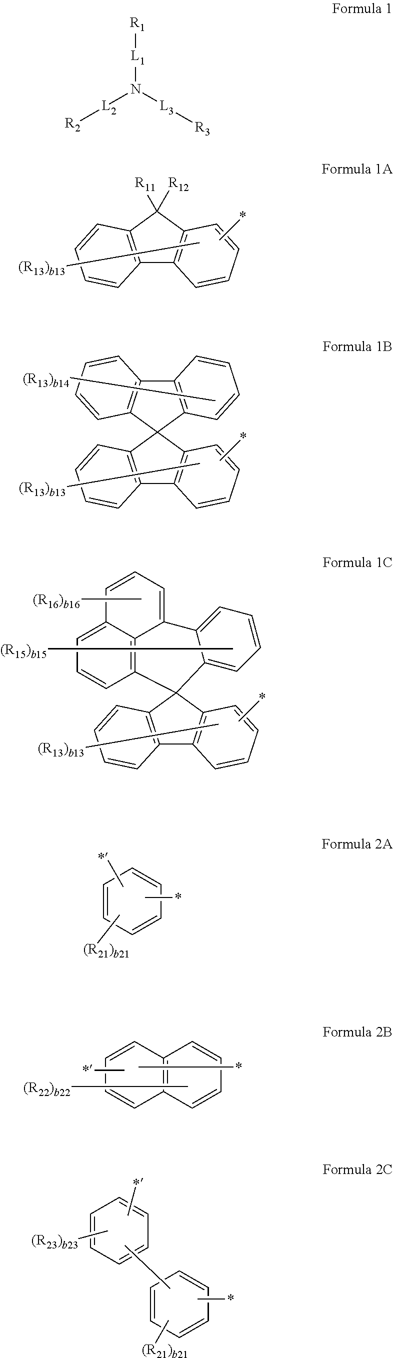

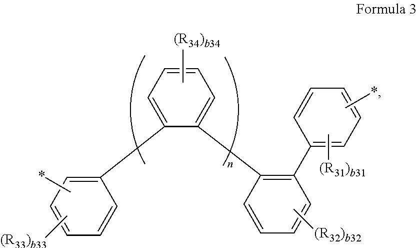

wherein, in Formula 1,
L₁ may be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
R₁ may be a group represented by one selected from among Formulae 1A to 1C, wherein, in Formulae 1A and 1C, * indicates a binding site to L₁ in Formula 1,
L₂ may be a group represented by one selected from among Formulae 2A to 2C, wherein, in Formulae 2A to 2C, * indicates a binding site to N in Formula 1, and indicates a binding site to R₂ in Formula 1,
R₂ may be a C₃-C₁₀ cycloalkyl group unsubstituted or substituted with at least one R₁₀ₐ,
L₃ may be a group represented by Formula 3, wherein, in Formula 3, * indicates a binding site to N in Formula 1, and *′ indicates a binding site to R₃ in Formula 1,
in Formula 3, n may be 0 or 1,
R₃, R₁₁ to R₁₆, R₂₁ to R₂₃, and R₃₁ to R₃₄ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ aryl alkyl group unsubstituted or substituted with at least one R₁₀ₐ, a C₂-C₆₀ heteroaryl alkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b13 and b15 may each be an integer from 0 to 7,
b14 may be an integer from 0 to 8,
b16 may be an integer from 0 to 3,
b21, b23, and b31 to b34 may each independently be an integer from 0 to 3,
b22 may be an integer from 0 to 6, and
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁ to Q₃, Q₁₁ to Q₁₃, Q₂₁ to Q₂₃, and Q₃₁ to Q₃₃ may each independently be hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments of the present disclosure, an electronic apparatus and electronic equipment may each include the light-emitting device.

According to one or more embodiments of the present disclosure, provided is the amine-containing compound represented by Formula 1.

### BRIEF DESCRIPTION OF THE DRAWINGS

The accompanying drawings are included to provide a further understanding of the present disclosure and are incorporated in and constitute a part of the present disclosure. The drawings illustrate embodiments of the present disclosure and, together with the description, serve to explain principles of the present disclosure. The above and other aspects, features, and advantages of certain embodiments of the disclosure will be more apparent from the following description taken in conjunction with the accompanying drawings, in which:

FIG. 1 is a schematic cross-sectional view of a light-emitting device according to one or more embodiments of the present disclosure;

FIG. 2 is a schematic cross-sectional view of an electronic apparatus according to one or more embodiments of the present disclosure;

FIG. 3 is a schematic cross-sectional view of an electronic apparatus according to one or more embodiments of the present disclosure;

FIG. 4 is a schematic view of electronic equipment according to one or more embodiments of the present disclosure;

FIG. 5 is a schematic view illustrating an exterior of a vehicle as electronic equipment according to one or more embodiments of present disclosure; and

FIGS. 6A to 6C are each a schematic view illustrating an interior of a vehicle as electronic equipment according to one or more embodiments of the present disclosure.

### DETAILED DESCRIPTION

Reference will now be made in more detail to embodiments, examples of which are illustrated in the accompanying drawings, wherein like reference numerals refer to like elements throughout the present disclosure, and duplicative descriptions thereof may not be provided for conciseness. In this regard, the embodiments of the present disclosure may have different forms and should not be construed as being limited to the descriptions set forth herein. Accordingly, the embodiments of the present disclosure are merely described, by referring to the drawings, to explain aspects of the present disclosure. As utilized herein, the term “and/or” or “or” may include any and all combinations of one or more of the associated listed items. Throughout the disclosure, the expressions such as “at least one of,” “one of,” and “selected from,” when preceding a list of elements, modify the entire list of elements and do not modify the individual elements of the list. For example, “at least one of a, b or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In one or more embodiments of the present disclosure, a light-emitting device may include: a first electrode; a second electrode facing the first electrode; an interlayer between the first electrode and the second electrode and including an emission layer; and an amine-containing compound represented by Formula 1:

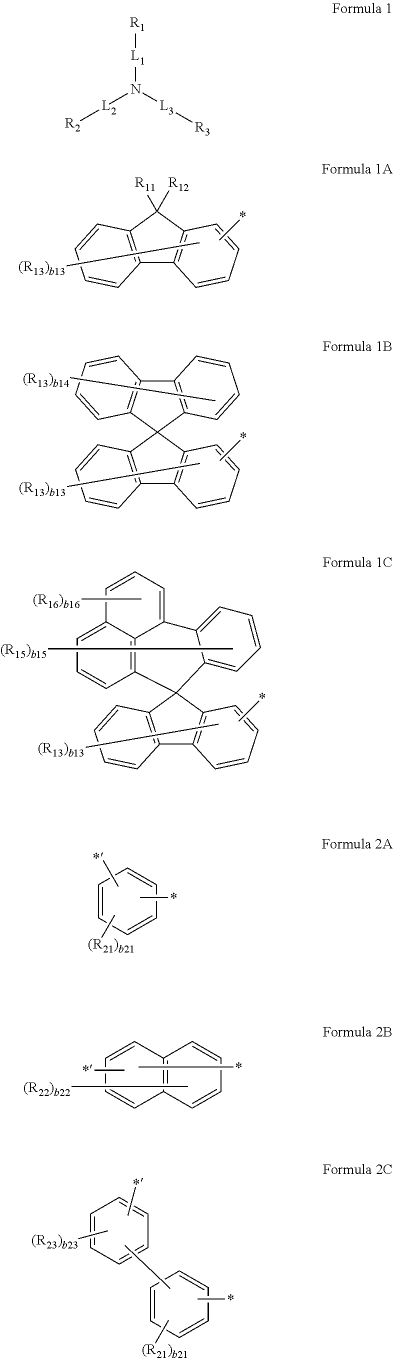

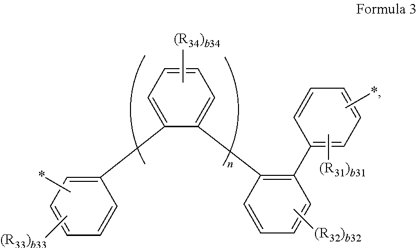

wherein, in Formula 1,
L₁ may be a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
In some embodiments, L₁ may be a single bond.
R₁ may be a group represented by one selected from among Formulae 1A to 1C, wherein, in Formulae 1A and 1C, * indicates a binding site to L₁ in Formula 1.
L₂ may be a group represented by one selected from among Formulae 2A to 2C, wherein, in Formulae 2A to 2C, * indicates a binding site to N in Formula 1, and indicates a binding site to R₂ in Formula 1.
R₂ may be a C₃-C₁₀ cycloalkyl group unsubstituted or substituted with at least one R₁₀ₐ.
L₃ may be a group represented by Formula 3, wherein, in Formula 3, * indicates a binding site to N in Formula 1, and *′ indicates a binding site to R₃ in Formula 1.
n in Formula 3 may be 0 or 1.
R₃, R₁₁ to R₁₆, R₂₁ to R₂₃, and R₃₁ to R₃₄ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ aryl alkyl group unsubstituted or substituted with at least one R₁₀ₐ, a C₂-C₆₀ heteroaryl alkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, R₃ may be hydrogen, a C₁-C₆₀ alkyl group unsubstituted or substituted with at least one R₁₀ₐ, or a C₃-C₆₀ carbocyclic group unsubstituted or substituted with at least one R₁₀ₐ.

In one or more embodiments, R₃ may be hydrogen.

In one or more embodiments, R₃ may be a C₃-C₁₀ cycloalkyl group unsubstituted or substituted with at least one R₁₀ₐ, and in some embodiments, R₃ may be a cyclopropyl group, a cyclobutyl group, a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, or a bicyclo[2.2.2]octyl group, each unsubstituted or substituted with at least one R₁₀ₐ.

In one or more embodiments, R₁₁ to R₁₆ may each independently be hydrogen, deuterium, —F, a cyano group, a C₁-C₂₀ alkyl group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ.

In one or more embodiments, R₁₁ to R₁₆ may each independently be: hydrogen, deuterium, —F, or a cyano group;
a C₁-C₂₀ alkyl group unsubstituted or substituted with hydrogen, deuterium, —F, a cyano group, or any combination thereof; or
a benzene group, a pyridine group, a dibenzofuran group, a dibenzothiophene group, a carbazole group, an azadibenzofuran group, an azadibenzothiophene group, or an azacarbazole group, each unsubstituted or substituted with hydrogen, deuterium, —F, a cyano group, a C₁-C₂₀ alkyl group, a deuterated C₁-C₂₀ alkyl group, a fluorinated C₁-C₂₀ alkyl group, a phenyl group, deuterated a phenyl group, a fluorinated phenyl group, a (C₁-C₂₀ alkyl)phenyl group, a biphenyl group, a dibenzofuranyl group, a dibenzothiophenyl group, a carbazolyl group, a fluorenyl group, a dibenzosilolyl group, or any combination thereof, and in some embodiments, R₁₁ to R₁₆ may each independently be:
hydrogen;
a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group; or
a benzene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, a 1,2-dimethylpropyl group, or a phenyl group, each unsubstituted or substituted with deuterium, —F, —Cl, —Br, —I, a hydroxyl group, a cyano group, or a nitro group.

In one or more embodiments, R₂₁ to R₂₃ and R₃₁ to R₃₄ may each independently be: hydrogen, deuterium, —F, —Cl, —Br, —I, a cyano group, or a C₁-C₂₀ alkyl group;
a C₁-C₂₀ alkyl group substituted with at least one selected from deuterium, —F, —Cl, —Br, —I, —CD₃, —CD₂H, —CDH₂, —CF₃, —CF₂H, —CFH₂, a cyano group, a nitro group, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a biphenyl group, a naphthyl group, a pyridinyl group, and a pyrimidinyl group;
a phenyl group, a pyridine group, a dibenzofuranyl group, a dibenzothiophenyl group, a carbazolyl group, an azadibenzofuranyl group, an azadibenzothiophenyl group, or an azacarbazolyl group, each unsubstituted or substituted with at least one selected from deuterium, —F, —Cl, —Br, —I, —CD₃, —CD₂H, —CDH₂, —CF₃, —CF₂H, —CFH₂, a cyano group, a C₁-C₂₀ alkyl group, a phenyl group, a biphenyl group, a (C₁-C₁₀ alkyl)phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₁)(Q₃₂)(Q₃₃), —N(Q₃₁)(Q₃₂), and —B(Q₃₁)(Q₃₂); or
—Si(Q₁)(Q₂)(Q₃), —N(Q₁)(Q₂), or —B(Q₁)(Q₂),
wherein Q₁ to Q₃ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, an isobutyl group, a sec-butyl group, a tert-butyl group, an n-pentyl group, an isopentyl group, a sec-pentyl group, a tert-pentyl group, a phenyl group, a naphthyl group, a pyridinyl group, a pyrimidinyl group, a pyridazinyl group, a pyrazinyl group, or a triazinyl group, each unsubstituted or substituted with at least one selected from deuterium, a C₁-C₁₀ alkyl group, a phenyl group, a biphenyl group, a pyridinyl group, a pyrimidinyl group, a pyridazinyl group, a pyrazinyl group, and a triazinyl group.

In some embodiments, R₂₁ to R₂₃ and R₃₁ to R₃₄ may each independently be:
a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, or a 1,2-dimethylpropyl group, each unsubstituted or substituted with deuterium, —F, —Cl, —Br, —I, a hydroxyl group, a cyano group, or a nitro group; or
a benzene group, a pyridine group, a dibenzofuran group, a dibenzothiophene group, a carbazole group, an azadibenzofuran group, an azadibenzothiophene group, or an azacarbazole group, each unsubstituted or substituted with a methyl group, an ethyl group, an n-propyl group, an iso-propyl group, an n-butyl group, an isobutyl group, a sec-butyl group, a tert-butyl group, an n-pentyl group, an isopentyl group, a sec-pentyl group, a tert-pentyl group, a 2-methylbutyl group, a 2-2 dimethylpropyl group, a 1-ethylpropyl group, a 1,2-dimethylpropyl group, or a phenyl group, each unsubstituted or substituted with deuterium, —F, —Cl, —Br, —I, a hydroxyl group, a cyano group, or a nitro group.

b13 and b15 may each be an integer from 0 to 7, b14 may be an integer from 0 to 8, b16 may be an integer from 0 to 3, b21, b23, and b31 to b34 may each independently be an integer from 0 to 3, and b22 may be an integer from 0 to 6.

In one or more embodiments, R₁ may be one selected from groups represented by Formulae 1-1 to 1-12:

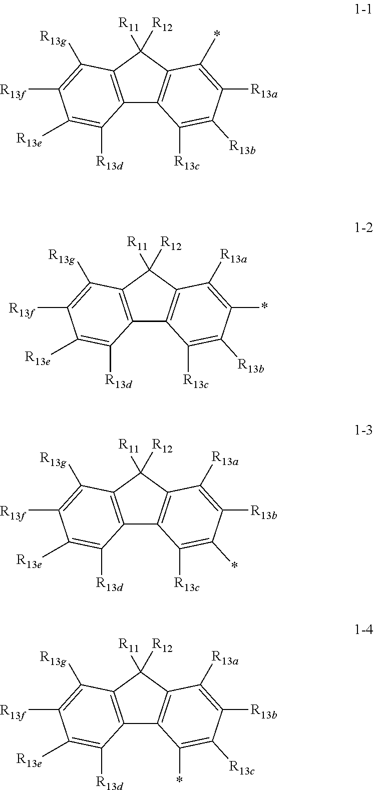

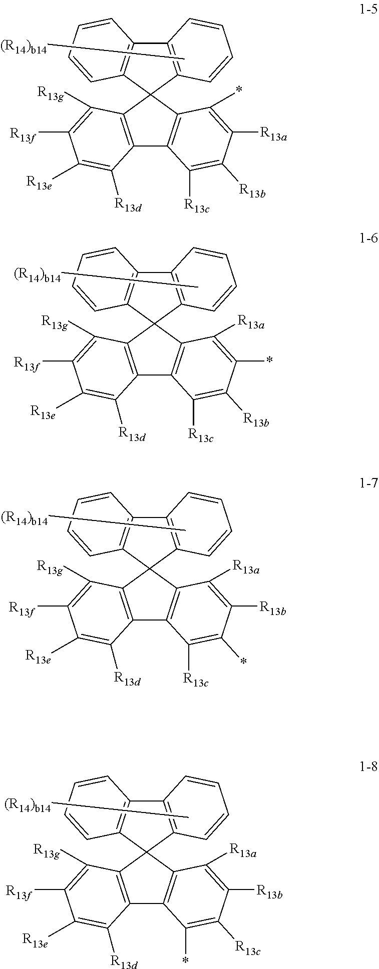

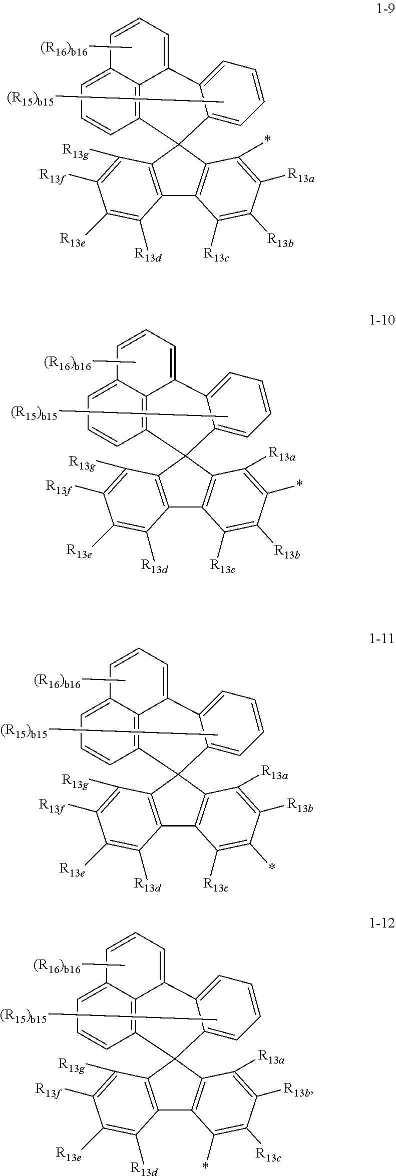

wherein in Formulae 11 to 1-12,
R₁₁, R₁₂, R₁₄, R₁₅, R₁₆, b14, b15, and b16 may respectively be understood by referring to the descriptions of R₁₁, R₁₂, R₁₄, R₁₅, R₁₆, b14, b15, and b16 provided herein,
R₁₃ₐ, R_{13b}, R_{13c}, R_{13d}, R₁₃ₑ, R_{13f}, and R_{13g} may each be understood by referring to the description of R₁₃ provided herein, and
indicates a binding site to Li in Formula 1.

In one or more embodiments, L₂ may be one selected from groups represented by Formulae 2-1 to 2-17:

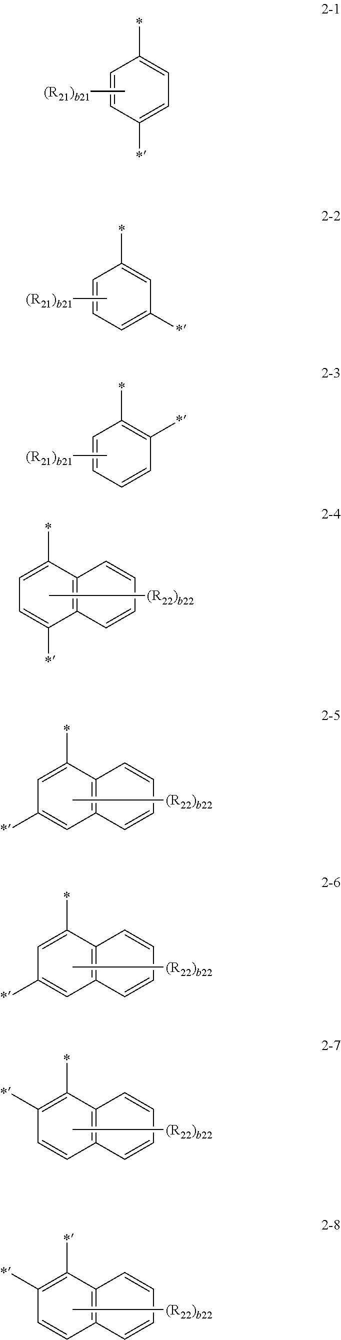

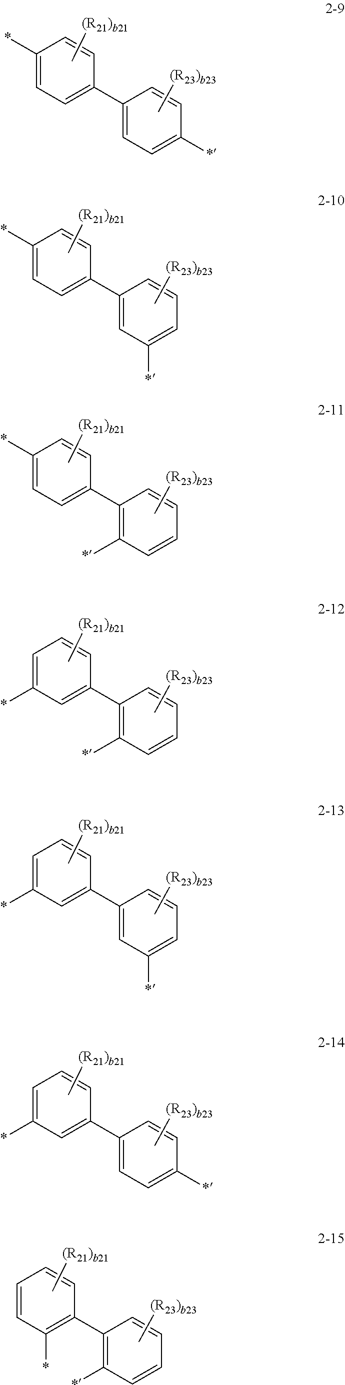

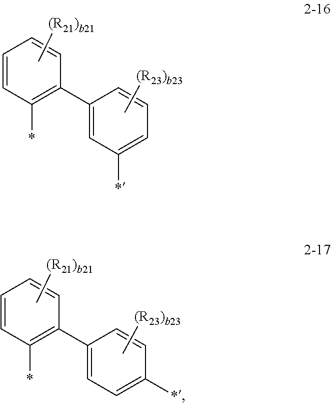

wherein, in Formulae 2-1 to 2-17,
R₂₁ to R₂₃ and b21 to b23 may respectively be understood by referring to the descriptions of R₂₁ to R₂₃ and b21 to b23 provided herein,
indicates a binding site to N in Formula 1, and
*′ indicates a binding site to R₂ in Formula 1.

In some embodiments, R₂ may be a cyclopropyl group, a cyclobutyl group, a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, or a bicyclo[2.2.2]octyl group, each unsubstituted or substituted with at least one R₁₀ₐ.

In one or more embodiments, R₂ may be one selected from Formulae 2-A to 2-G:

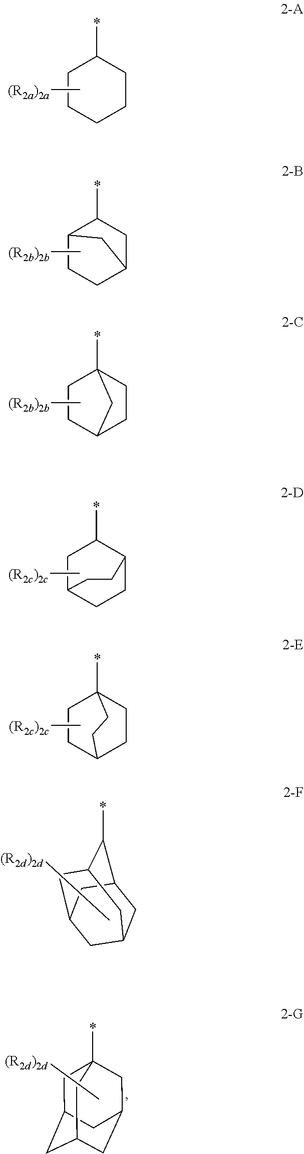

wherein, in Formulae 2-A to 2-G,
2a and 2b may each be an integer from 0 to 11,
2c may be an integer from 0 to 13,
2d may be an integer from 0 to 15,
R₂ₐ to R_{2d} may respectively be understood by referring to the descriptions of R₃, R₁₁ to R₁₆, R₂₁ to R₂₃, and R₃₁ to R₃₄ provided herein, and
* indicates a binding site to L₂ in Formula 1.

Any hydrogen in a cyclohexyl group of Formula 2-A may be substituted with R₂ₐ other than hydrogen,

Any hydrogen in a norbornanyl group of Formulae 2-B and 2-C may be substituted with R_{2b} other than hydrogen,

Any hydrogen in a bicyclo[2.2.2]octyl group of Formulae 2-D and 2-E may be substituted with R_{2c} other than hydrogen, and

Any hydrogen in an adamantanyl group of Formulae 2-F and 2-G may be substituted with R_{2d} other than hydrogen.

In one or more embodiments, L₃ may be one selected from groups represented by Formulae 3-1 to 3-18:

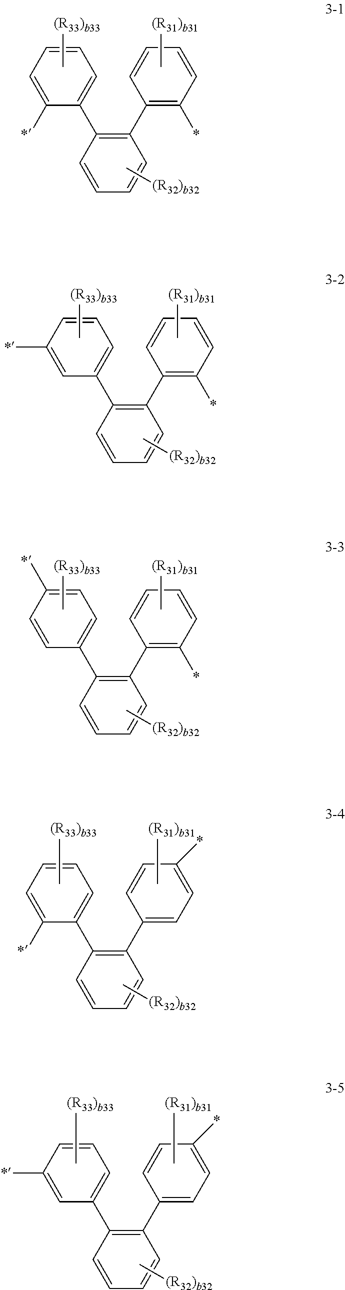

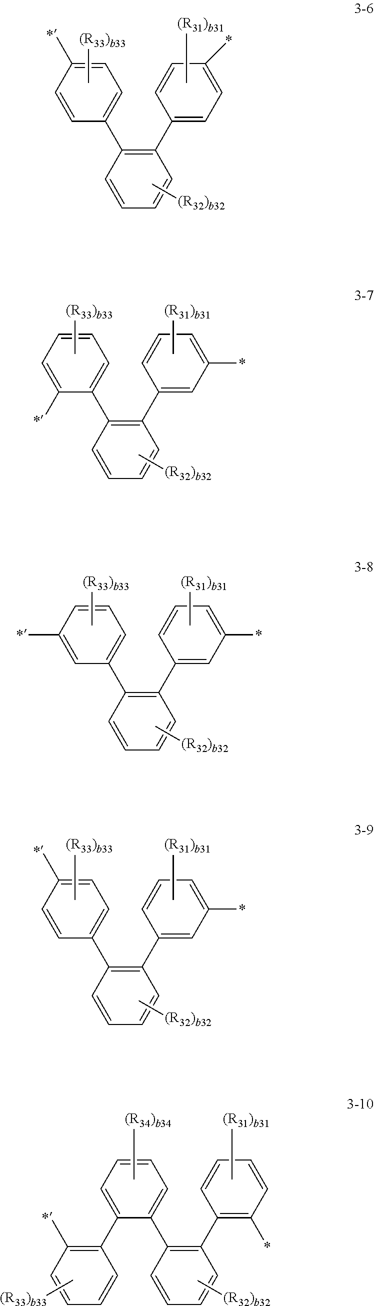

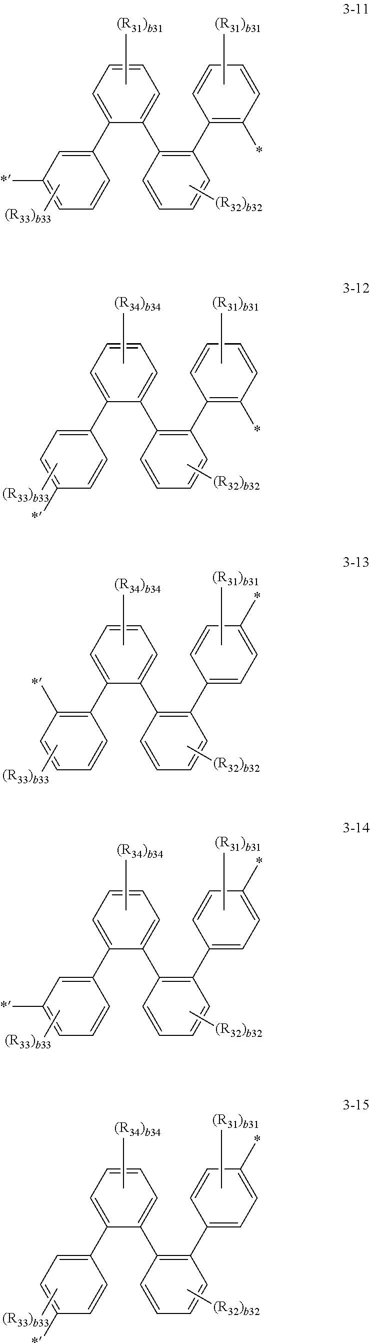

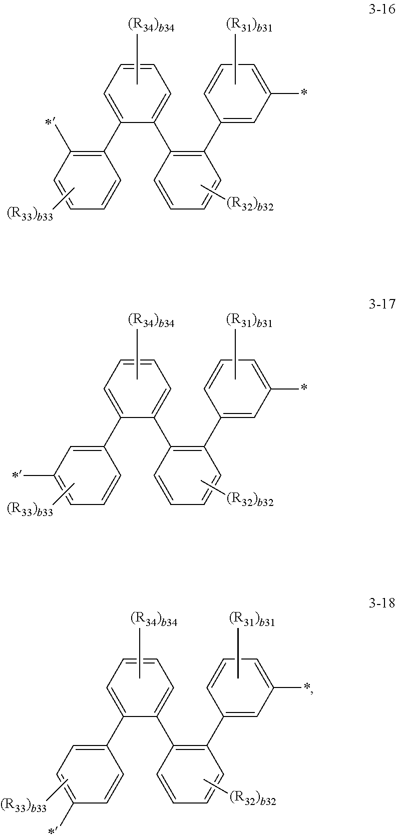

wherein, in Formulae 3-1 to 3-18,
R₃₁ to R₃₄ and b31 to b34 may respectively be understood by referring to the descriptions of R₃₁ to R₃₄ and b31 to b34 provided herein, and
* indicates a binding site to N in Formula 1, and *′ indicates a binding site to R₃ in Formula 1.

In one or more embodiments, the amine-containing compound represented by Formula 1 may be one selected from Compounds 1 to 375:

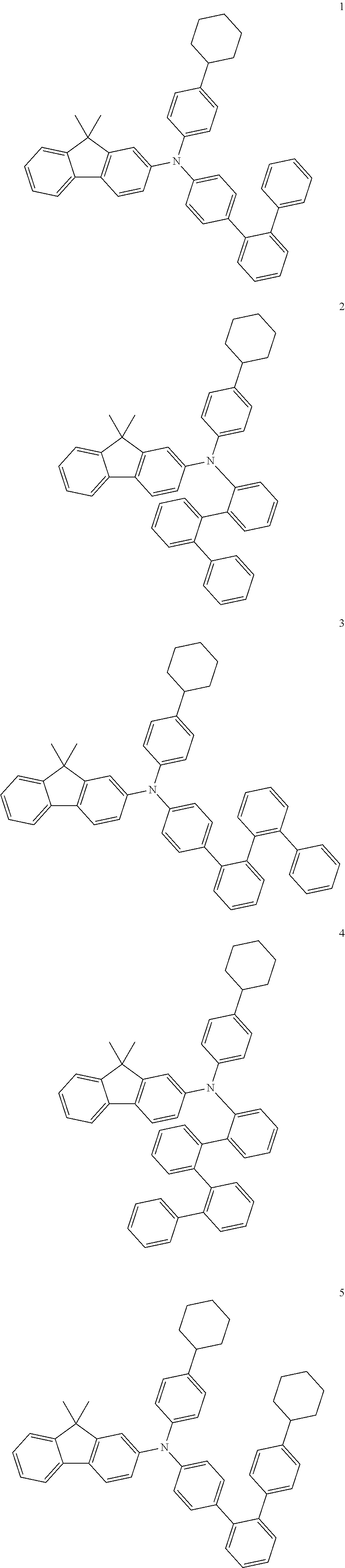

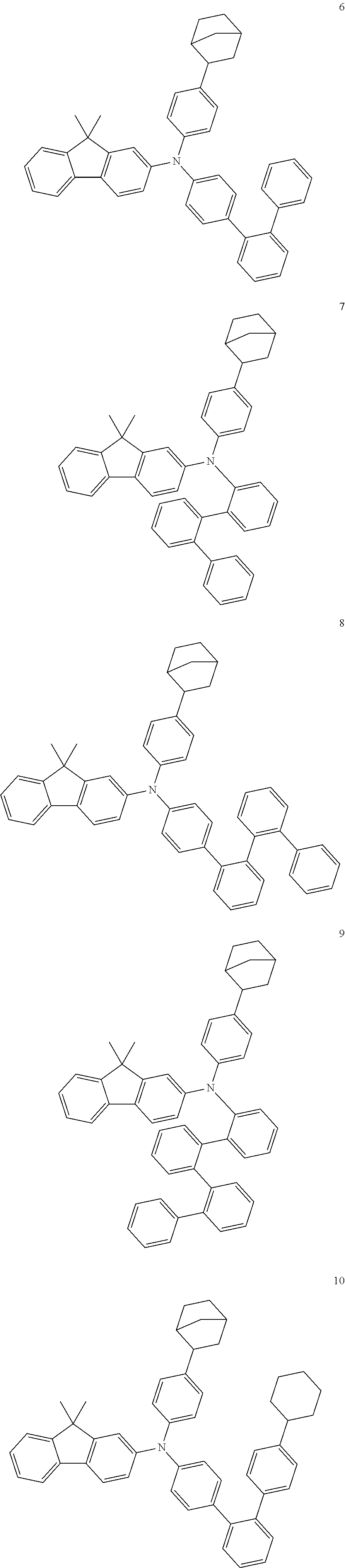

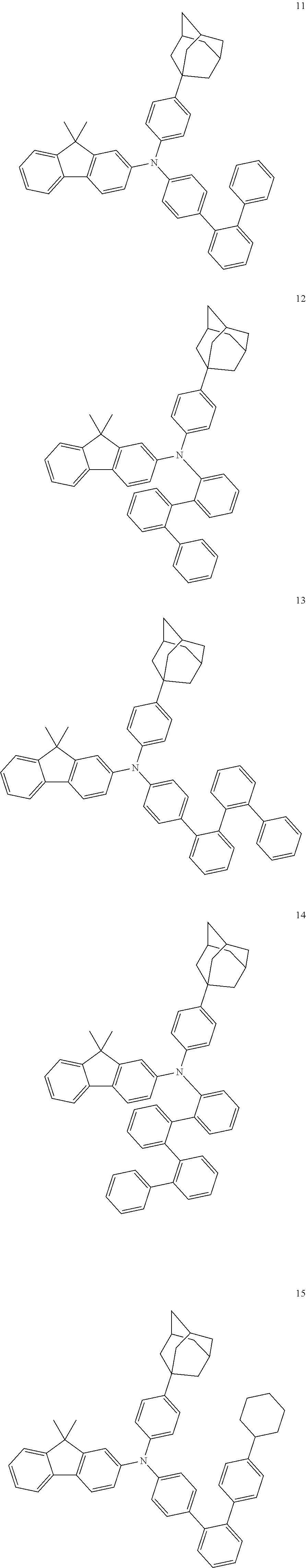 [SMILES: CC1(C)c2ccccc2-c2ccc(N(c3ccc(-c4ccccc4-c4ccccc4)cc3)c3ccc(C45CC6CC(CC(C6)C4)C5)cc3)cc21 ; CC1(C)c2ccccc2-c2ccc(N(c3ccc(C45CC6CC(CC(C6)C4)C5)cc3)c3ccccc3-c3ccccc3-c3ccccc3)cc21 ; CC1(C)c2ccccc2-c2ccc(N(c3ccc(-c4ccccc4-c4ccccc4-c4ccccc4)cc3)c3ccc(C45CC6CC(CC(C6)C4)C5)cc3)cc21 ; CC1(C)c2ccccc2-c2ccc(N(c3ccc(C45CC6CC(CC(C6)C4)C5)cc3)c3ccccc3-c3ccccc3-c3ccccc3-c3ccccc3)cc21 ; CC1(C)c2ccccc2-c2ccc(N(c3ccc(-c4ccccc4-c4ccc(C5CCCCC5)cc4)cc3)c3ccc(C45CC6CC(CC(C6)C4)C5)cc3)cc21]

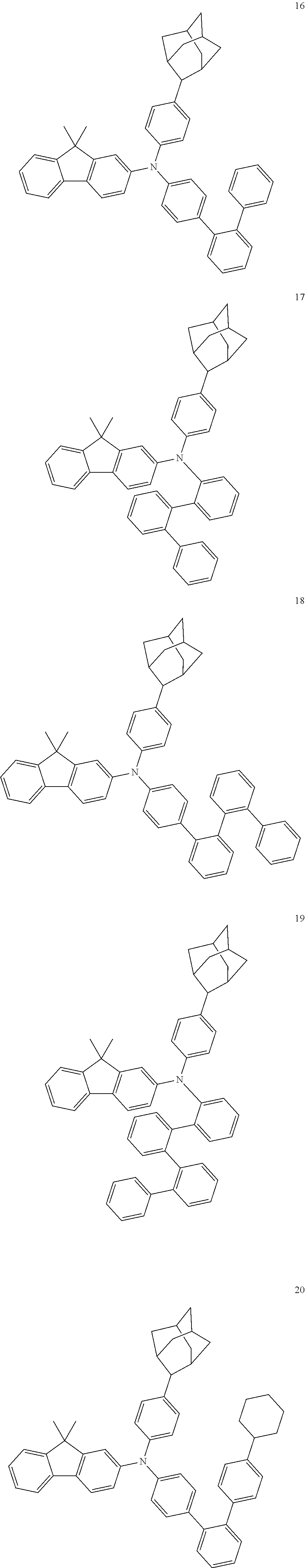

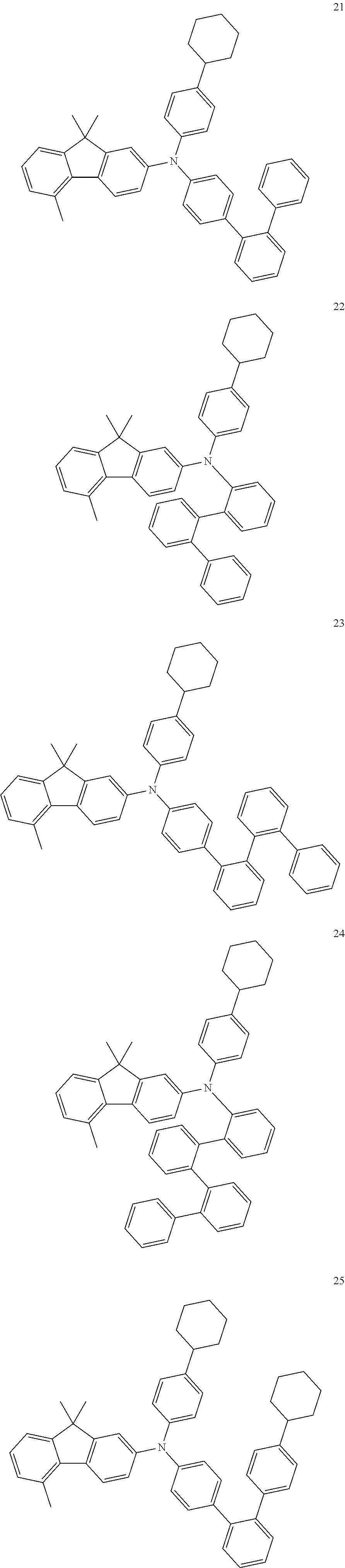

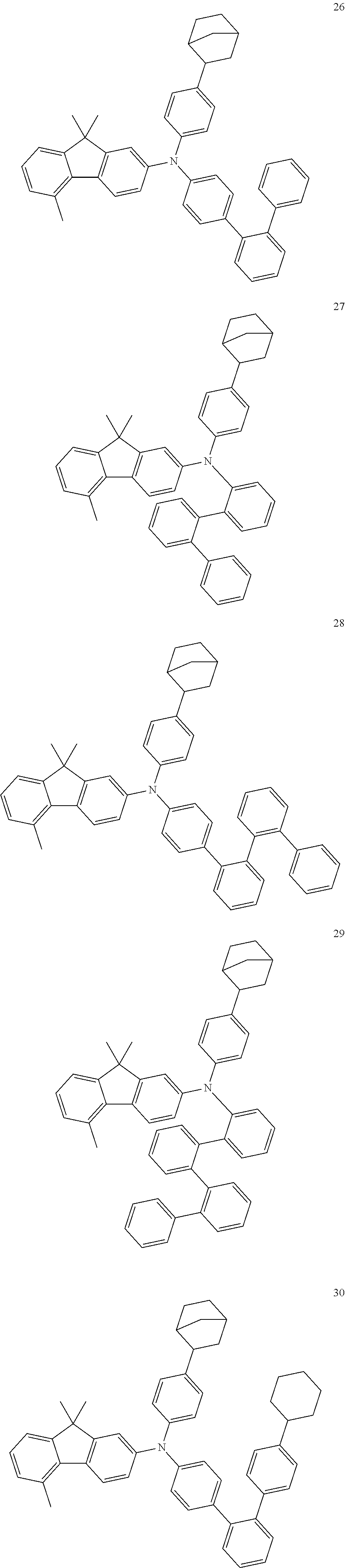

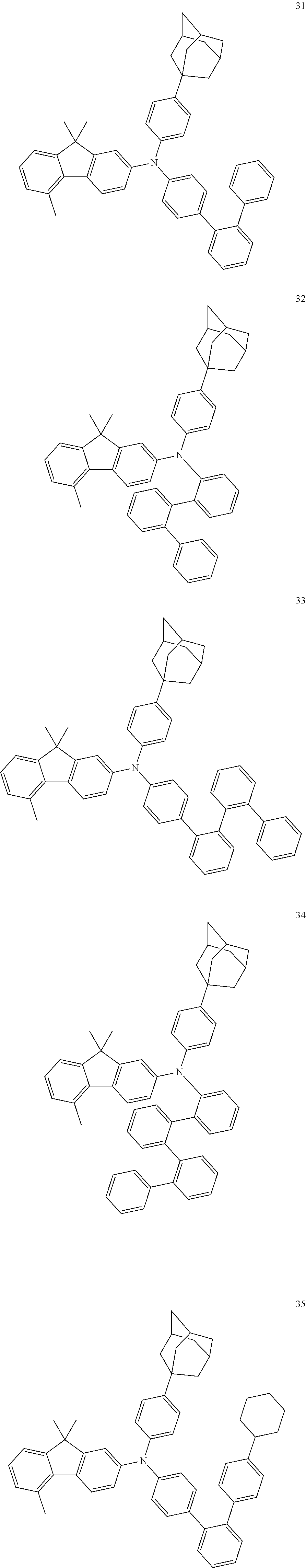 [SMILES: Cc1cccc2c1-c1ccc(N(c3ccc(-c4ccccc4-c4ccccc4)cc3)c3ccc(C45CC6CC(CC(C6)C4)C5)cc3)cc1C2(C)C ; Cc1cccc2c1-c1ccc(N(c3ccc(C45CC6CC(CC(C6)C4)C5)cc3)c3ccccc3-c3ccccc3-c3ccccc3)cc1C2(C)C ; Cc1cccc2c1-c1ccc(N(c3ccc(-c4ccccc4-c4ccccc4-c4ccccc4)cc3)c3ccc(C45CC6CC(CC(C6)C4)C5)cc3)cc1C2(C)C ; Cc1cccc2c1-c1ccc(N(c3ccc(C45CC6CC(CC(C6)C4)C5)cc3)c3ccccc3-c3ccccc3-c3ccccc3-c3ccccc3)cc1C2(C)C ; Cc1cccc2c1-c1ccc(N(c3ccc(-c4ccccc4-c4ccc(C5CCCCC5)cc4)cc3)c3ccc(C45CC6CC(CC(C6)C4)C5)cc3)cc1C2(C)C]

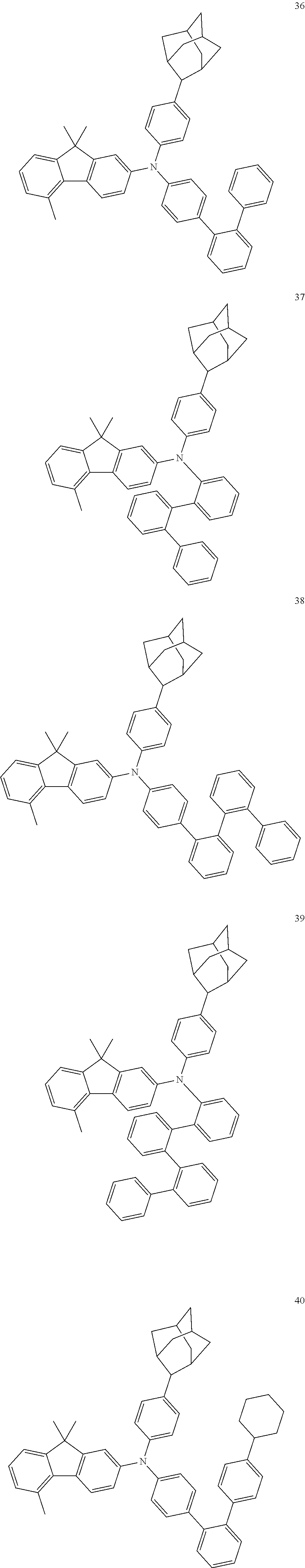

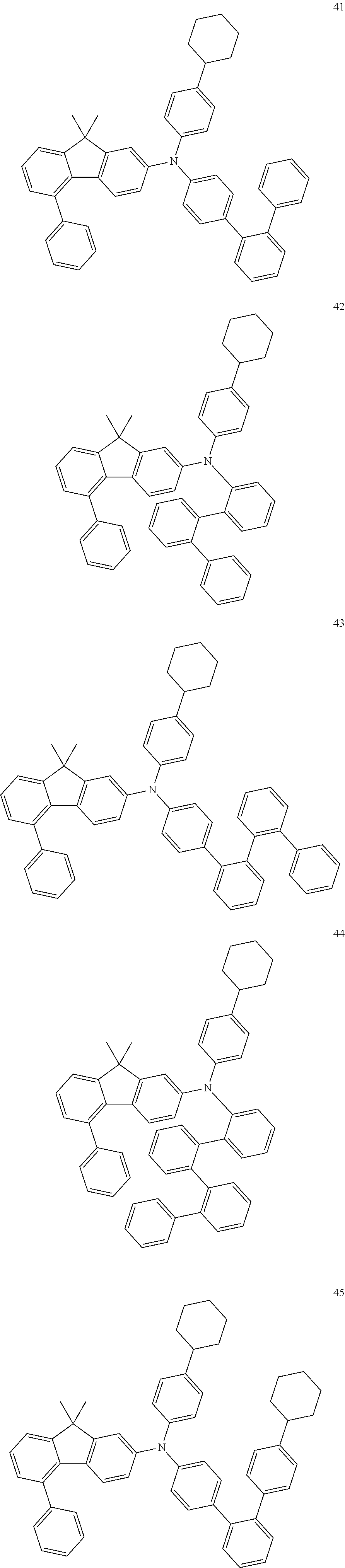

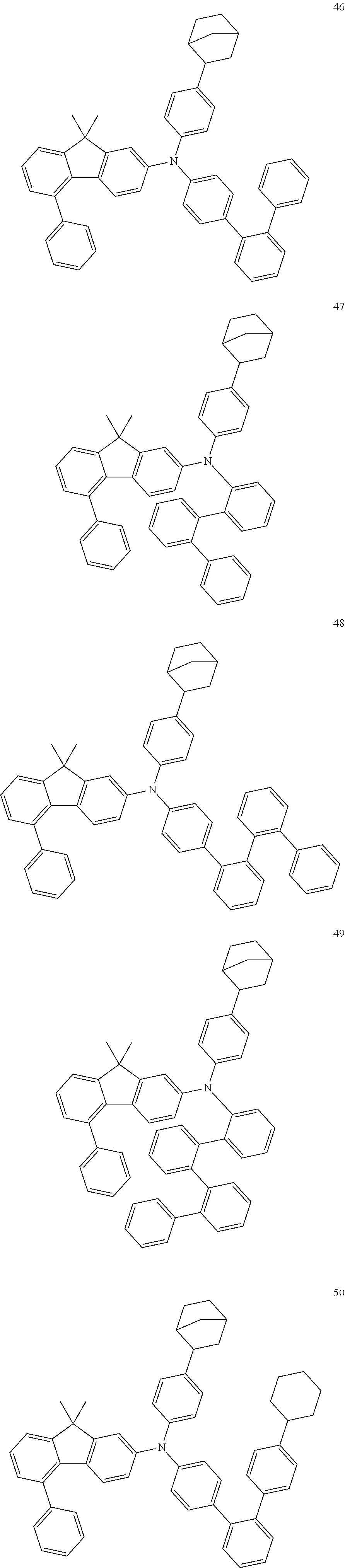

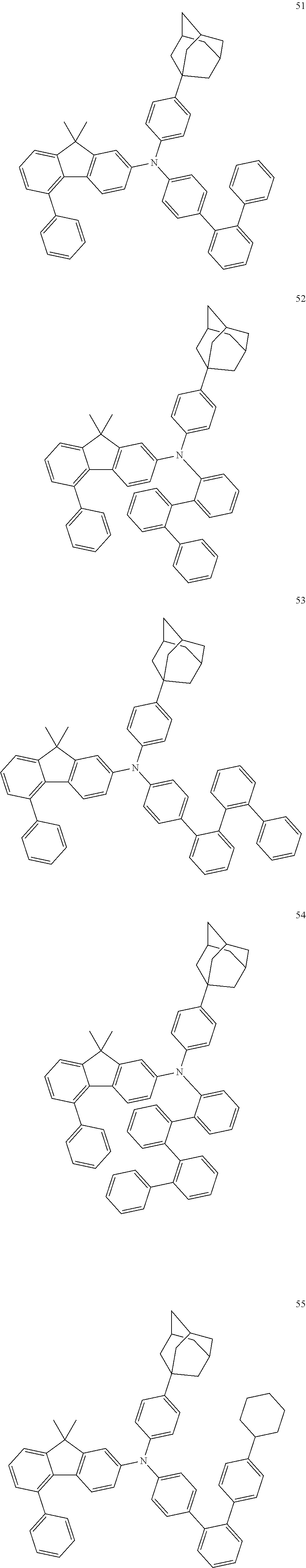

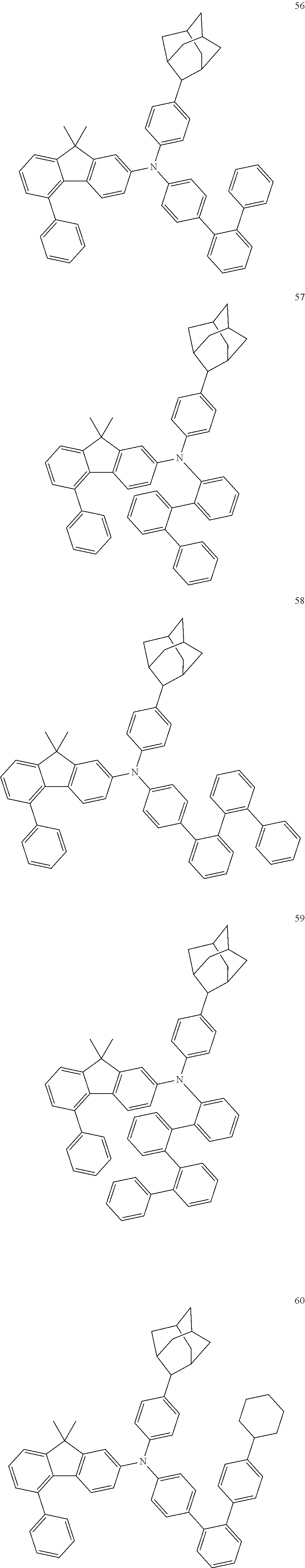

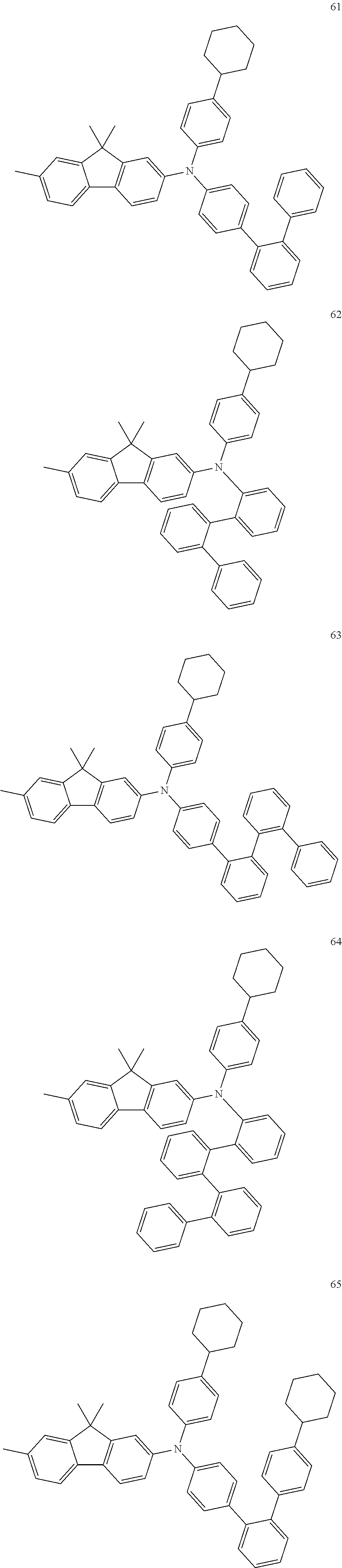

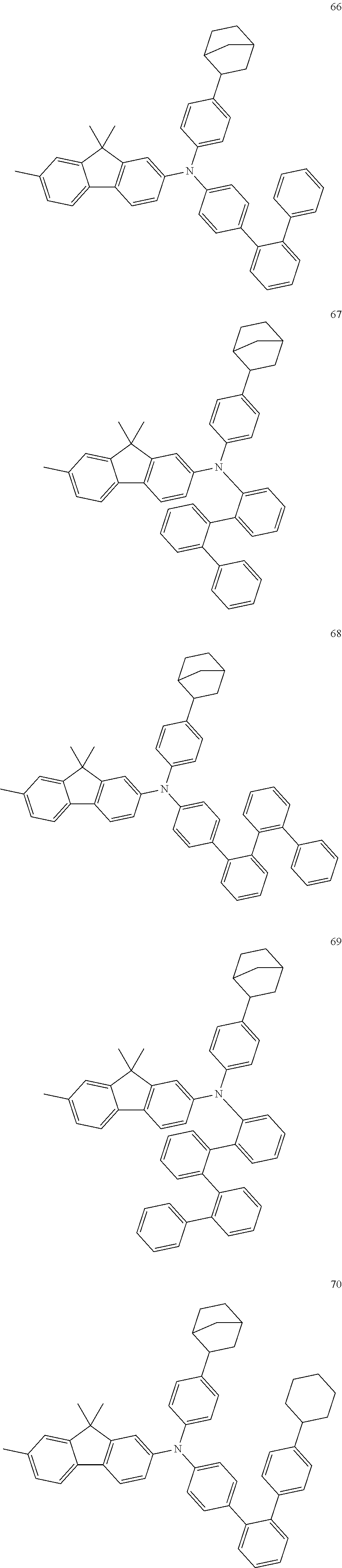

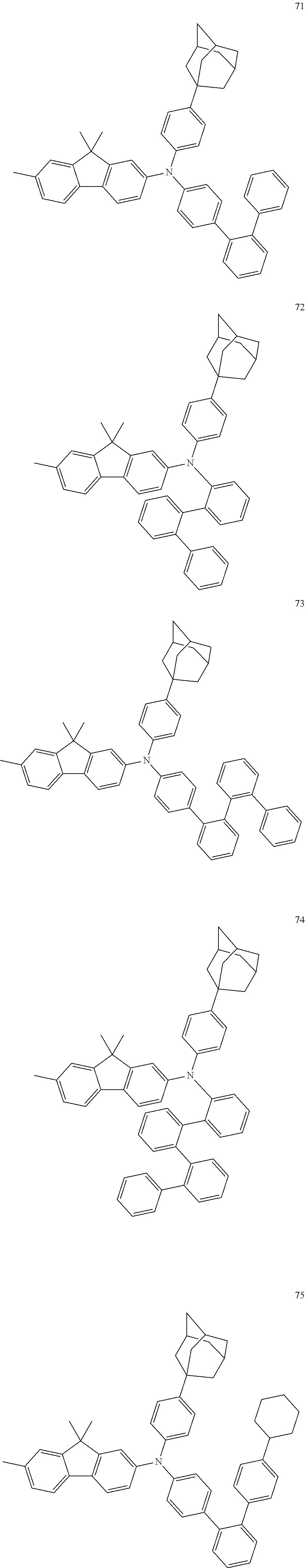 [SMILES: Cc1ccc2c(c1)C(C)(C)c1cc(N(c3ccc(-c4ccccc4-c4ccccc4)cc3)c3ccc(C45CC6CC(CC(C6)C4)C5)cc3)ccc1-2 ; Cc1ccc2c(c1)C(C)(C)c1cc(N(c3ccc(C45CC6CC(CC(C6)C4)C5)cc3)c3ccccc3-c3ccccc3-c3ccccc3)ccc1-2 ; Cc1ccc2c(c1)C(C)(C)c1cc(N(c3ccc(-c4ccccc4-c4ccccc4-c4ccccc4)cc3)c3ccc(C45CC6CC(CC(C6)C4)C5)cc3)ccc1-2 ; Cc1ccc2c(c1)C(C)(C)c1cc(N(c3ccc(C45CC6CC(CC(C6)C4)C5)cc3)c3ccccc3-c3ccccc3-c3ccccc3-c3ccccc3)ccc1-2 ; Cc1ccc2c(c1)C(C)(C)c1cc(N(c3ccc(-c4ccccc4-c4ccc(C5CCCCC5)cc4)cc3)c3ccc(C45CC6CC(CC(C6)C4)C5)cc3)ccc1-2]

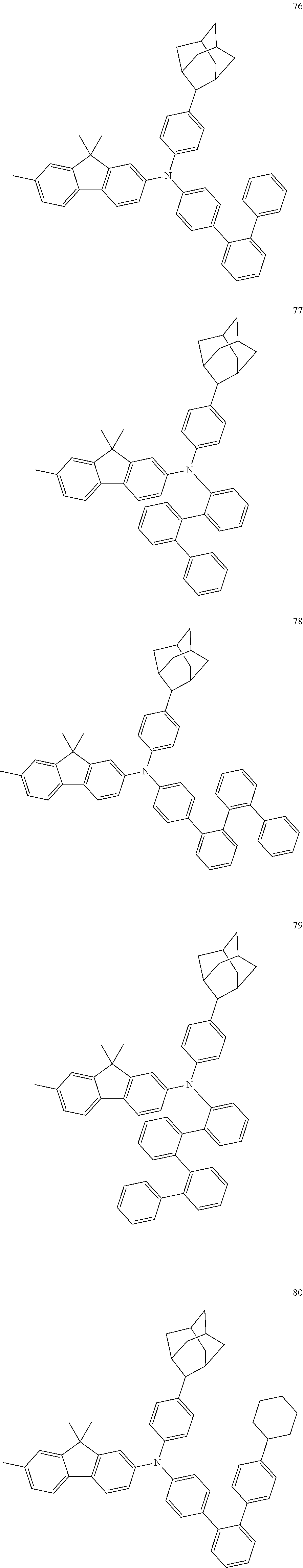 [SMILES: Cc1ccc2c(c1)C(C)(C)c1cc(N(c3ccc(-c4ccccc4-c4ccccc4)cc3)c3ccc(C4C5CC6CC(C5)CC4C6)cc3)ccc1-2 ; Cc1ccc2c(c1)C(C)(C)c1cc(N(c3ccc(C4C5CC6CC(C5)CC4C6)cc3)c3ccccc3-c3ccccc3-c3ccccc3)ccc1-2 ; Cc1ccc2c(c1)C(C)(C)c1cc(N(c3ccc(-c4ccccc4-c4ccccc4-c4ccccc4)cc3)c3ccc(C4C5CC6CC(C5)CC4C6)cc3)ccc1-2 ; Cc1ccc2c(c1)C(C)(C)c1cc(N(c3ccc(C4C5CC6CC(C5)CC4C6)cc3)c3ccccc3-c3ccccc3-c3ccccc3-c3ccccc3)ccc1-2 ; Cc1ccc2c(c1)C(C)(C)c1cc(N(c3ccc(-c4ccccc4-c4ccc(C5CCCCC5)cc4)cc3)c3ccc(C4C5CC6CC(C5)CC4C6)cc3)ccc1-2]

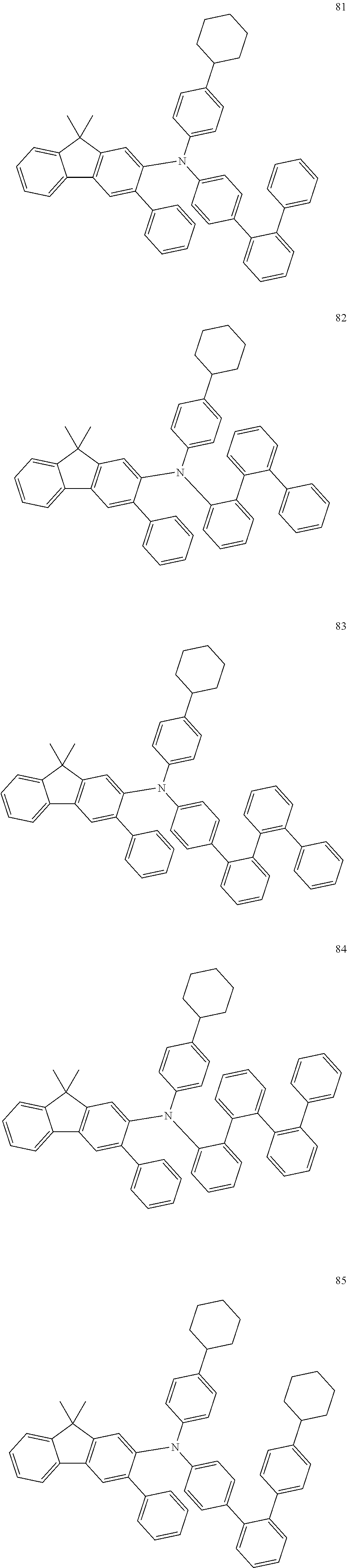 [SMILES: CC1(C)c2ccccc2-c2cc(-c3ccccc3)c(N(c3ccc(-c4ccccc4-c4ccccc4)cc3)c3ccc(C4CCCCC4)cc3)cc21 ; CC1(C)c2ccccc2-c2cc(-c3ccccc3)c(N(c3ccc(C4CCCCC4)cc3)c3ccccc3-c3ccccc3-c3ccccc3)cc21 ; CC1(C)c2ccccc2-c2cc(-c3ccccc3)c(N(c3ccc(-c4ccccc4-c4ccccc4-c4ccccc4)cc3)c3ccc(C4CCCCC4)cc3)cc21 ; CC1(C)c2ccccc2-c2cc(-c3ccccc3)c(N(c3ccc(C4CCCCC4)cc3)c3ccccc3-c3ccccc3-c3ccccc3-c3ccccc3)cc21 ; CC1(C)c2ccccc2-c2cc(-c3ccccc3)c(N(c3ccc(-c4ccccc4-c4ccc(C5CCCCC5)cc4)cc3)c3ccc(C4CCCCC4)cc3)cc21]

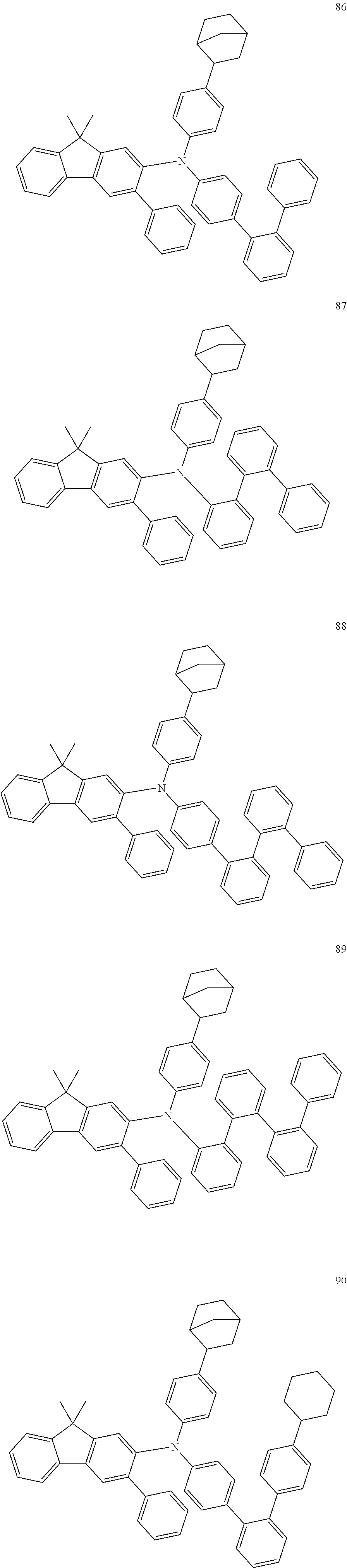 [SMILES: CC1(C)c2ccccc2-c2cc(-c3ccccc3)c(N(c3ccc(-c4ccccc4-c4ccccc4)cc3)c3ccc(C4CC5CCC4C5)cc3)cc21 ; CC1(C)c2ccccc2-c2cc(-c3ccccc3)c(N(c3ccc(C4CC5CCC4C5)cc3)c3ccccc3-c3ccccc3-c3ccccc3)cc21 ; CC1(C)c2ccccc2-c2cc(-c3ccccc3)c(N(c3ccc(-c4ccccc4-c4ccccc4-c4ccccc4)cc3)c3ccc(C4CC5CCC4C5)cc3)cc21 ; CC1(C)c2ccccc2-c2cc(-c3ccccc3)c(N(c3ccc(C4CC5CCC4C5)cc3)c3ccccc3-c3ccccc3-c3ccccc3-c3ccccc3)cc21 ; CC1(C)c2ccccc2-c2cc(-c3ccccc3)c(N(c3ccc(-c4ccccc4-c4ccc(C5CCCCC5)cc4)cc3)c3ccc(C4CC5CCC4C5)cc3)cc21]

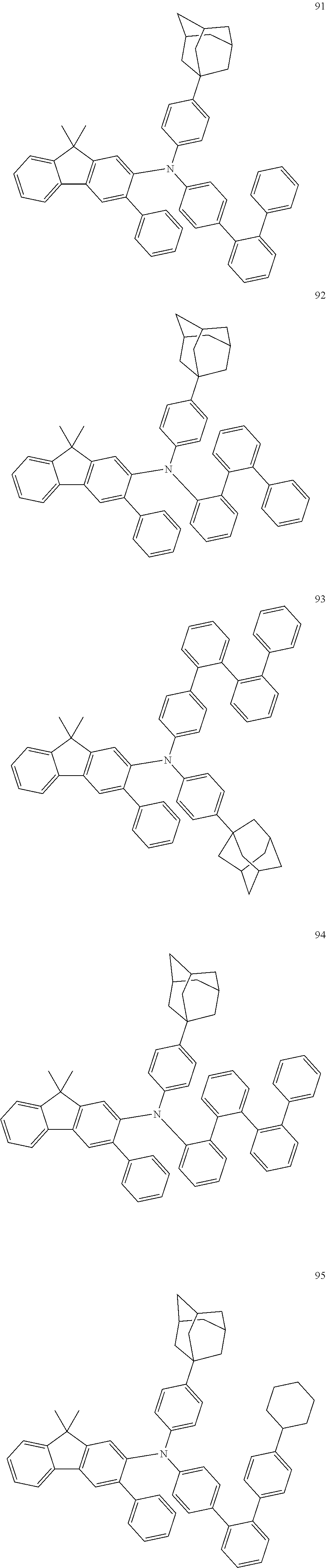

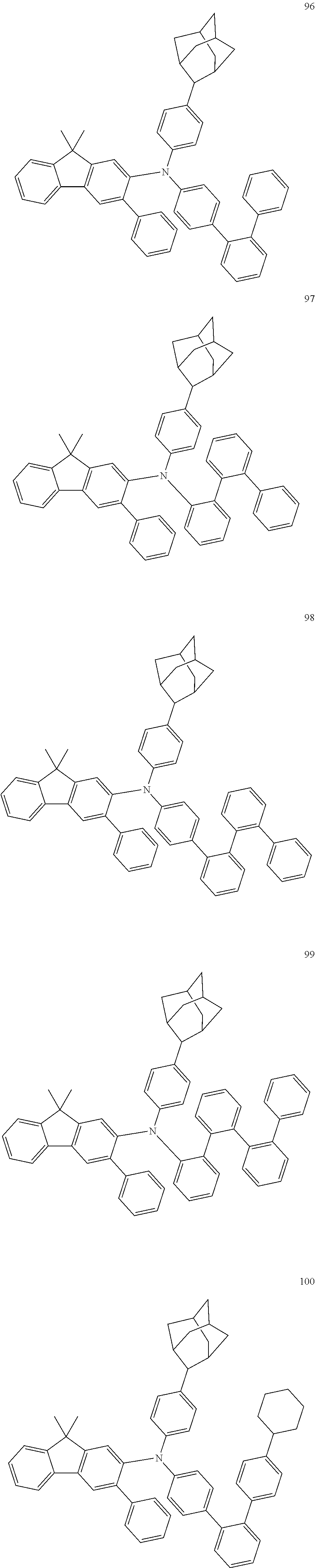

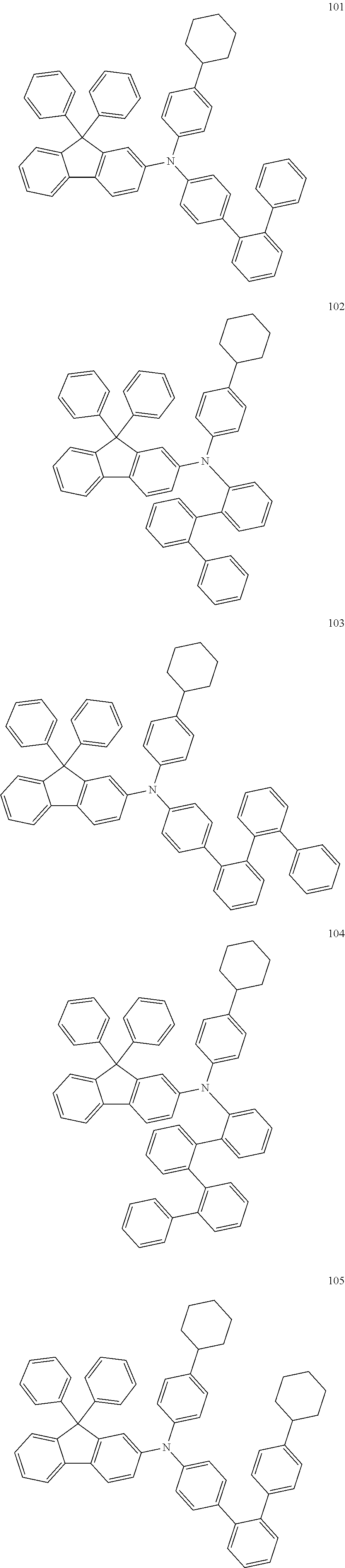

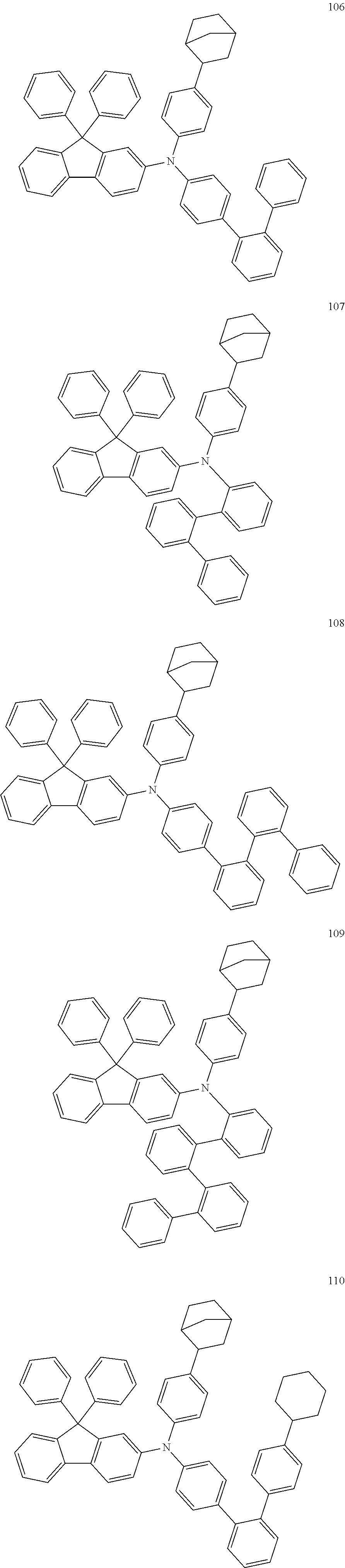

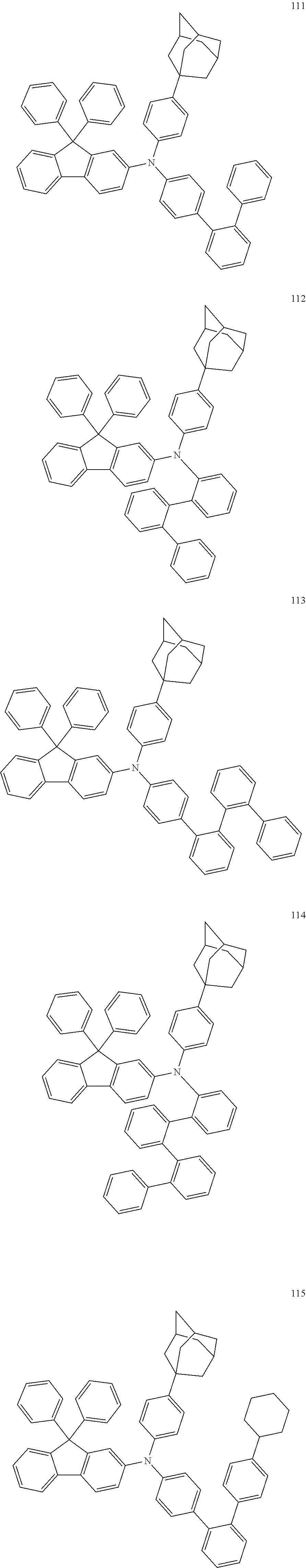

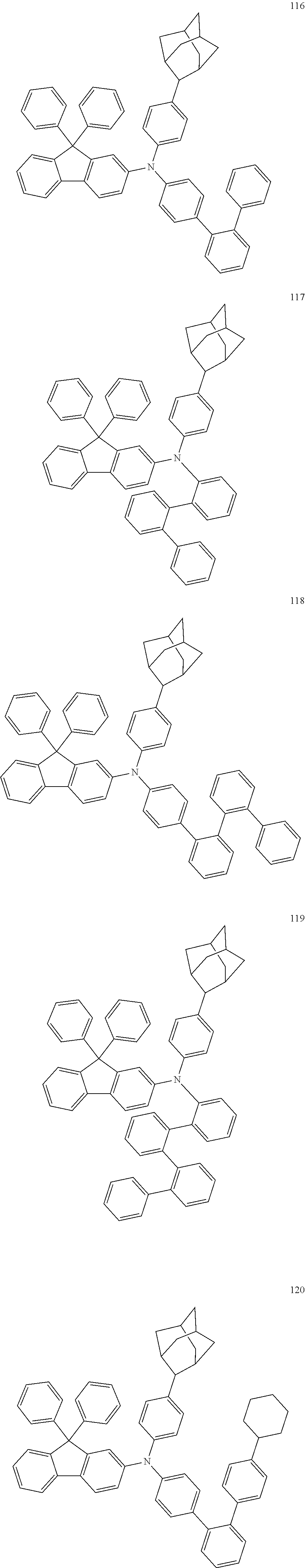

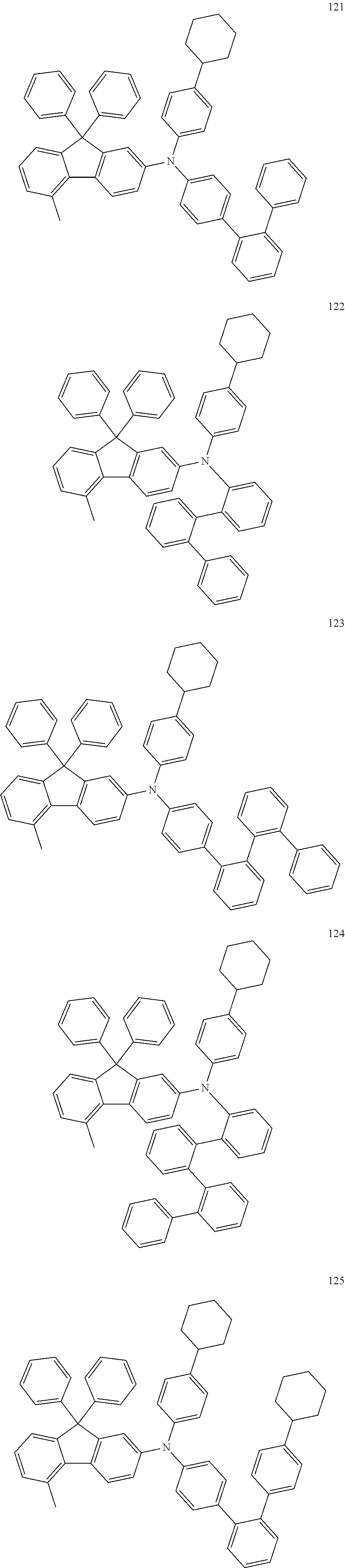

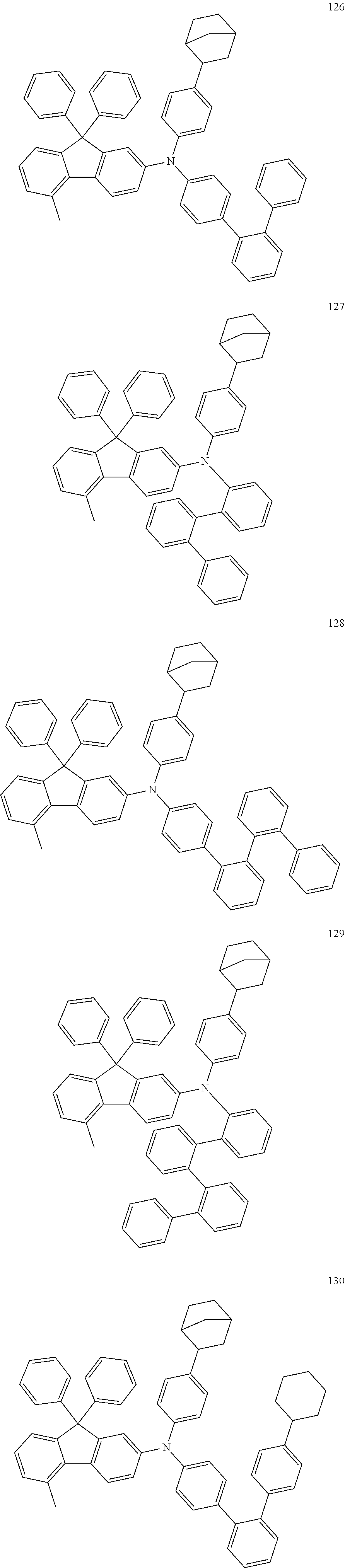

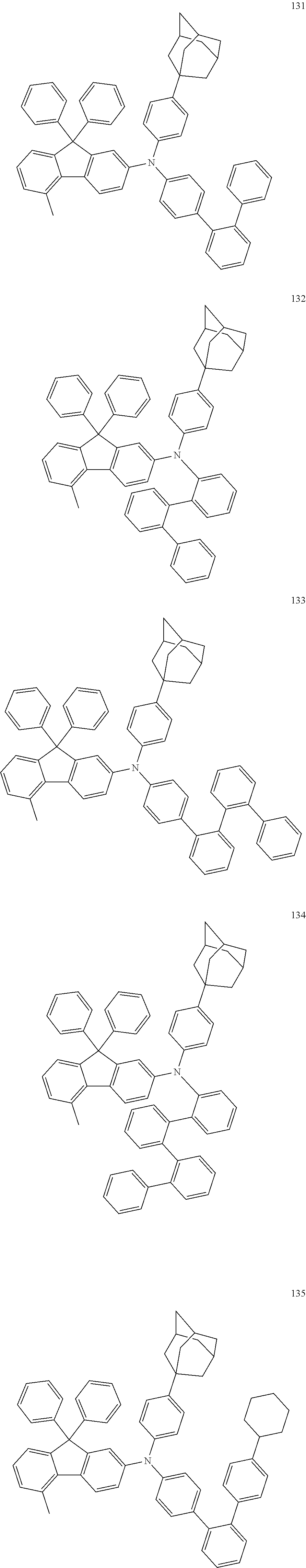

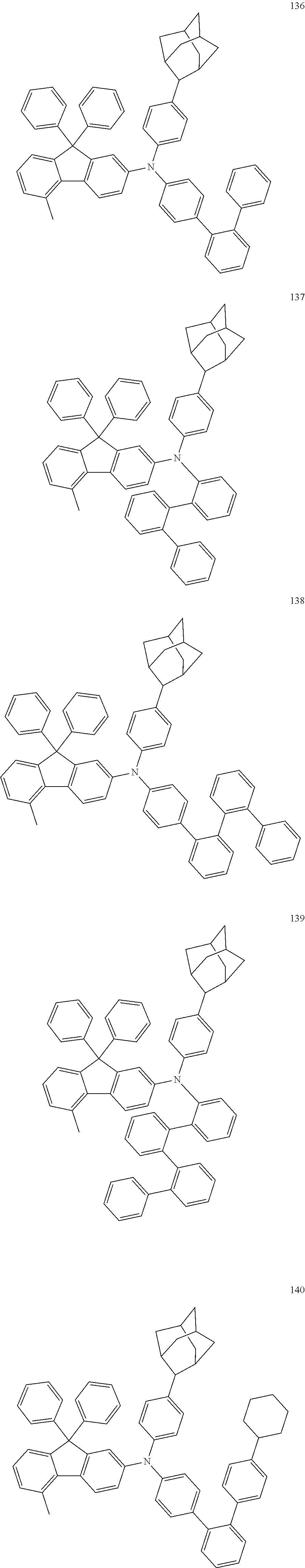

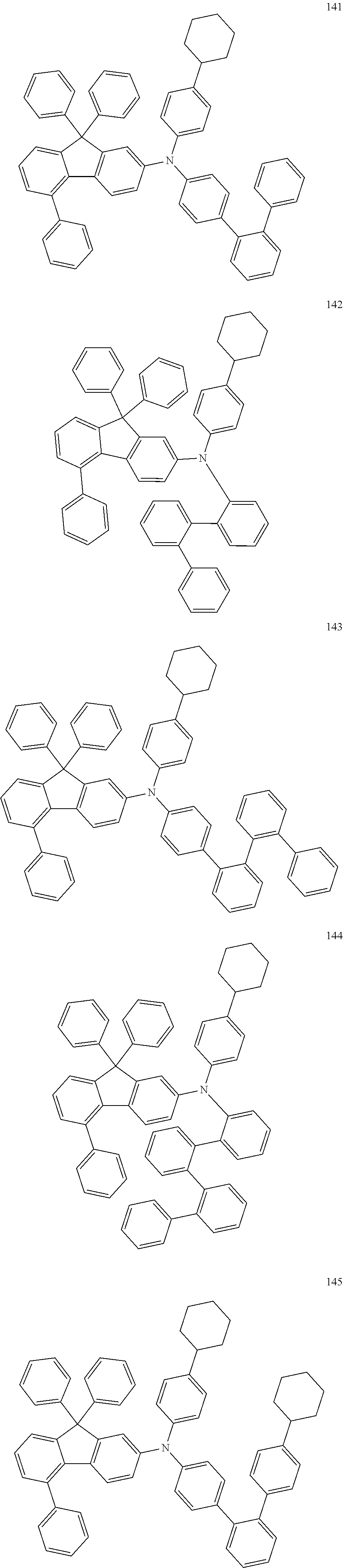

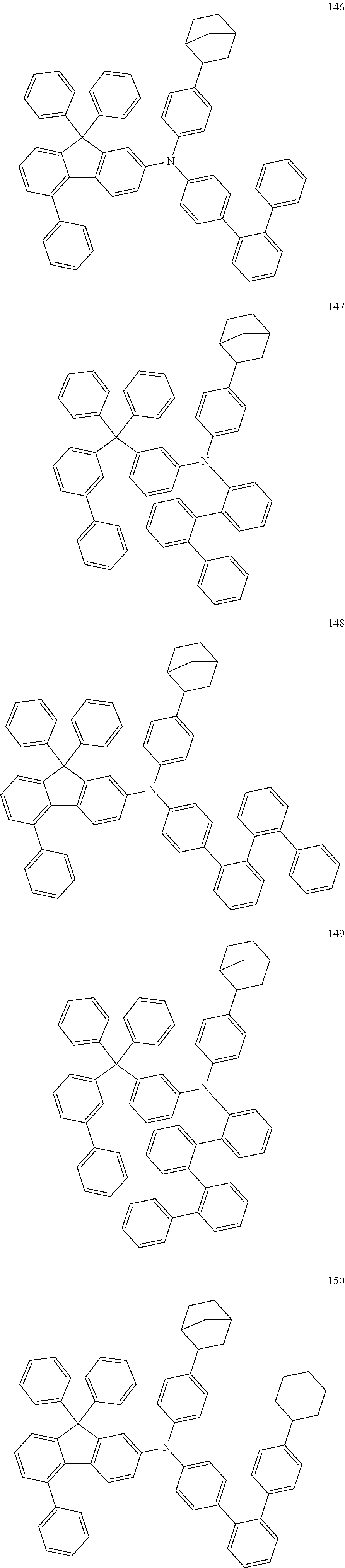

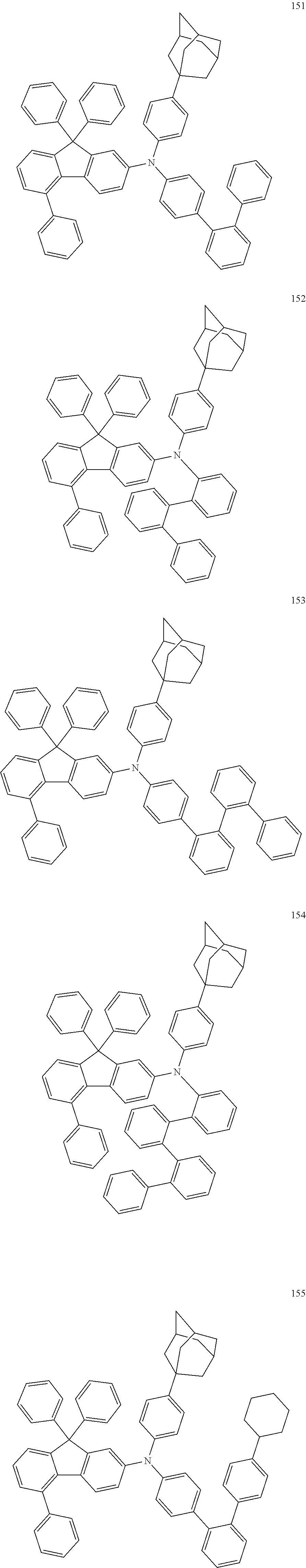

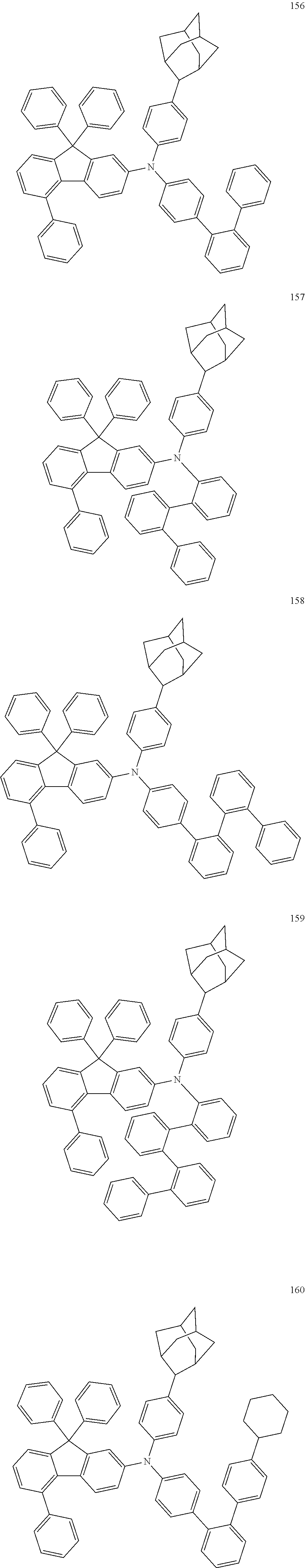

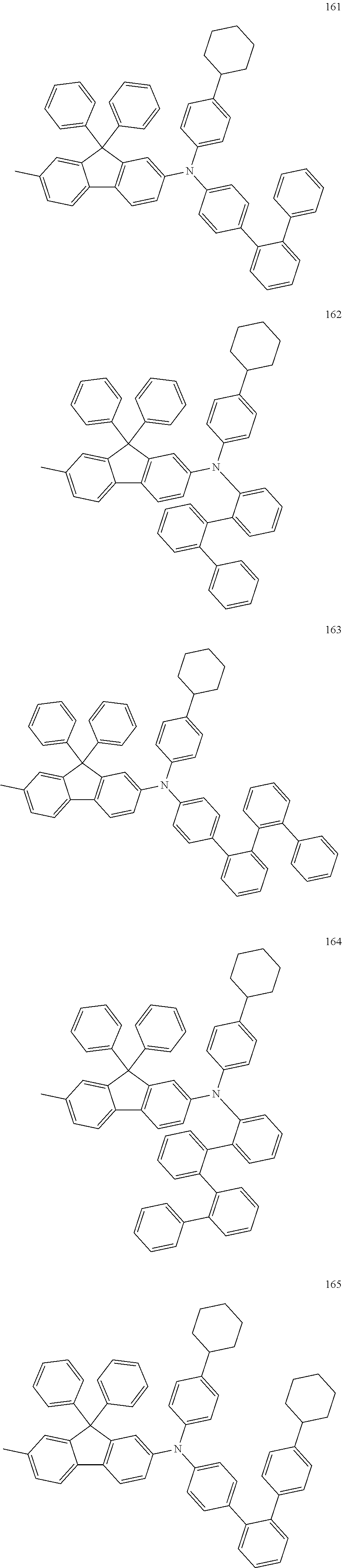

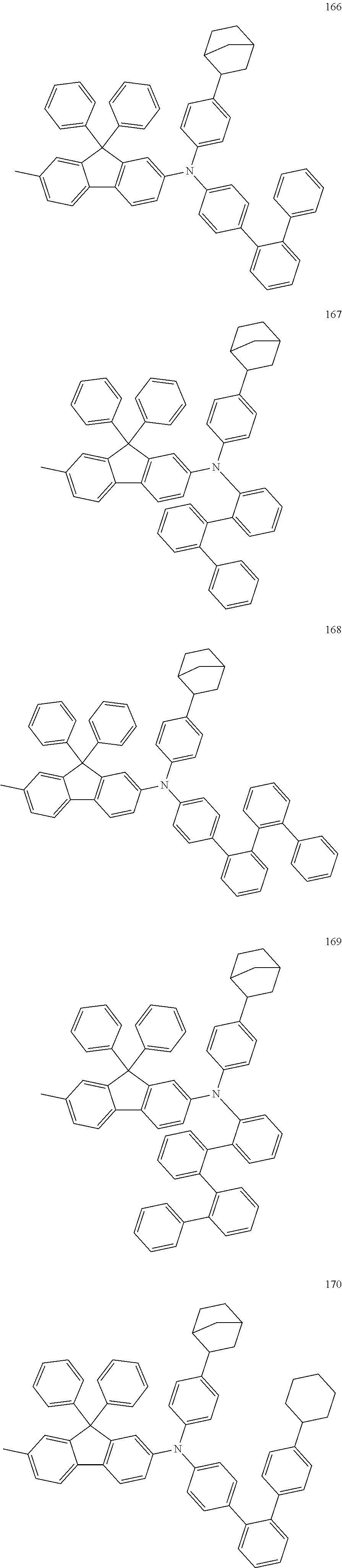

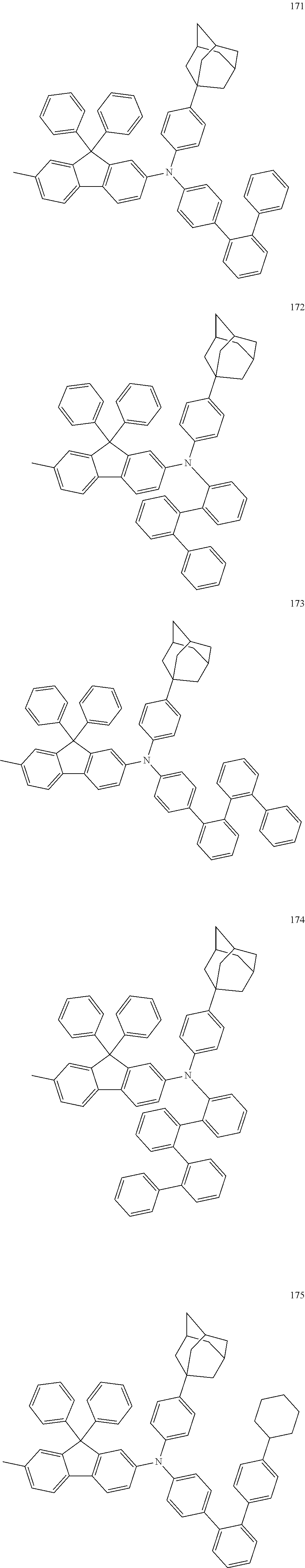

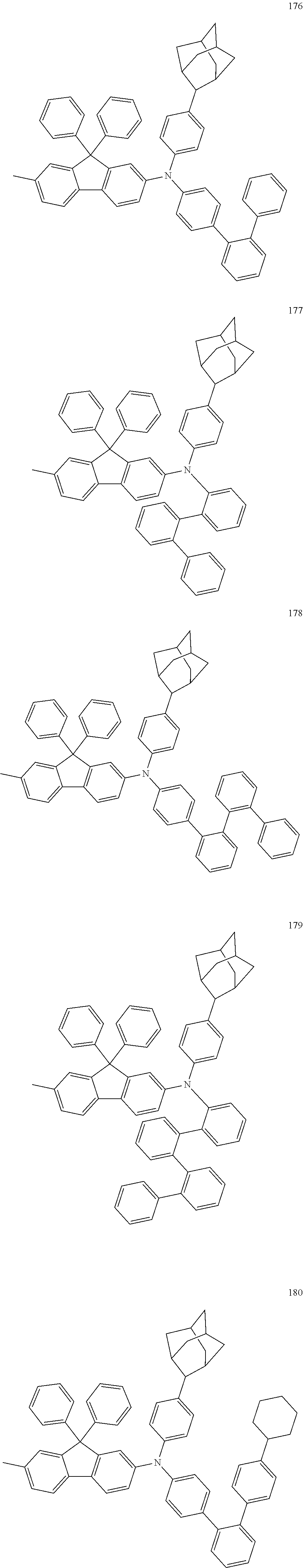

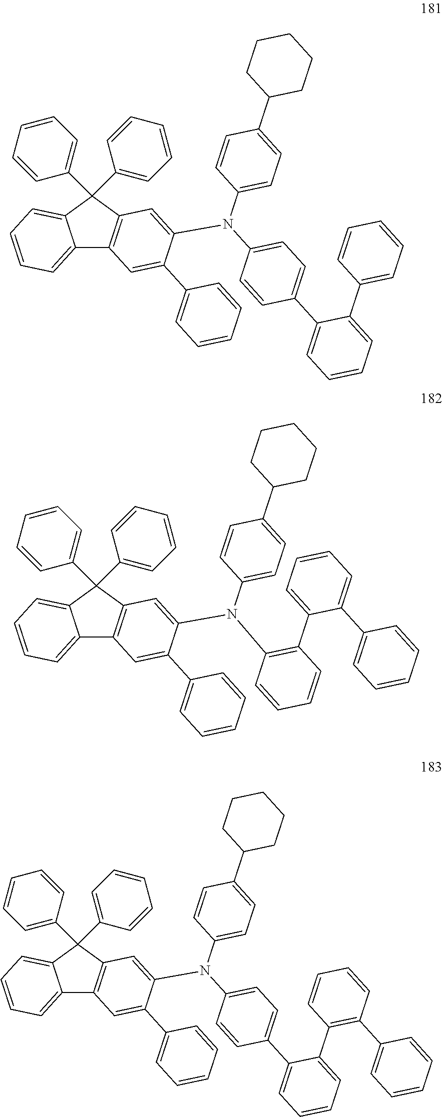 [SMILES: c1ccc(-c2ccccc2-c2ccc(N(c3ccc(C4CCCCC4)cc3)c3cc4c(cc3-c3ccccc3)-c3ccccc3C4(c3ccccc3)c3ccccc3)cc2)cc1 ; c1ccc(-c2ccccc2-c2ccccc2N(c2ccc(C3CCCCC3)cc2)c2cc3c(cc2-c2ccccc2)-c2ccccc2C3(c2ccccc2)c2ccccc2)cc1 ; c1ccc(-c2ccccc2-c2ccccc2-c2ccc(N(c3ccc(C4CCCCC4)cc3)c3cc4c(cc3-c3ccccc3)-c3ccccc3C4(c3ccccc3)c3ccccc3)cc2)cc1]

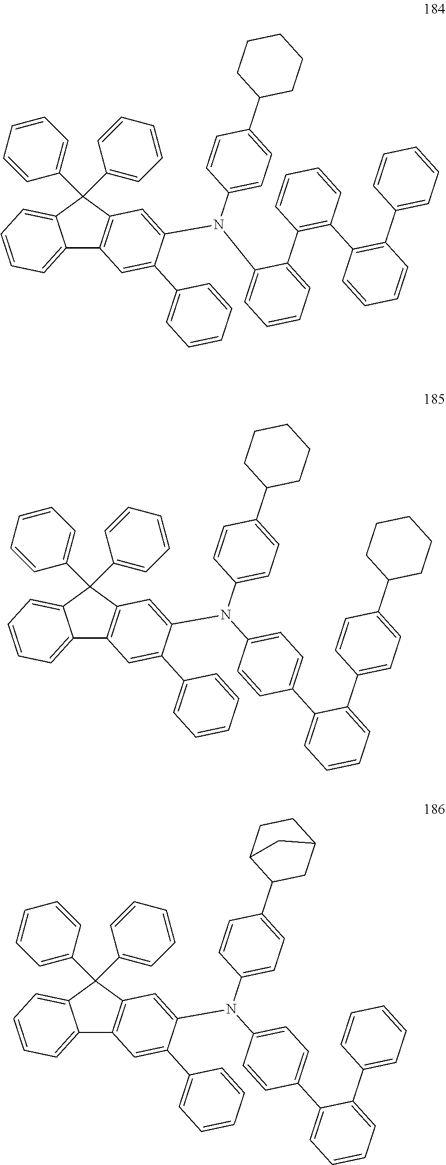 [SMILES: c1ccc(-c2ccccc2-c2ccccc2-c2ccccc2N(c2ccc(C3CCCCC3)cc2)c2cc3c(cc2-c2ccccc2)-c2ccccc2C3(c2ccccc2)c2ccccc2)cc1 ; c1ccc(-c2cc3c(cc2N(c2ccc(-c4ccccc4-c4ccc(C5CCCCC5)cc4)cc2)c2ccc(C4CCCCC4)cc2)C(c2ccccc2)(c2ccccc2)c2ccccc2-3)cc1 ; c1ccc(-c2ccccc2-c2ccc(N(c3ccc(C4CC5CCC4C5)cc3)c3cc4c(cc3-c3ccccc3)-c3ccccc3C4(c3ccccc3)c3ccccc3)cc2)cc1]

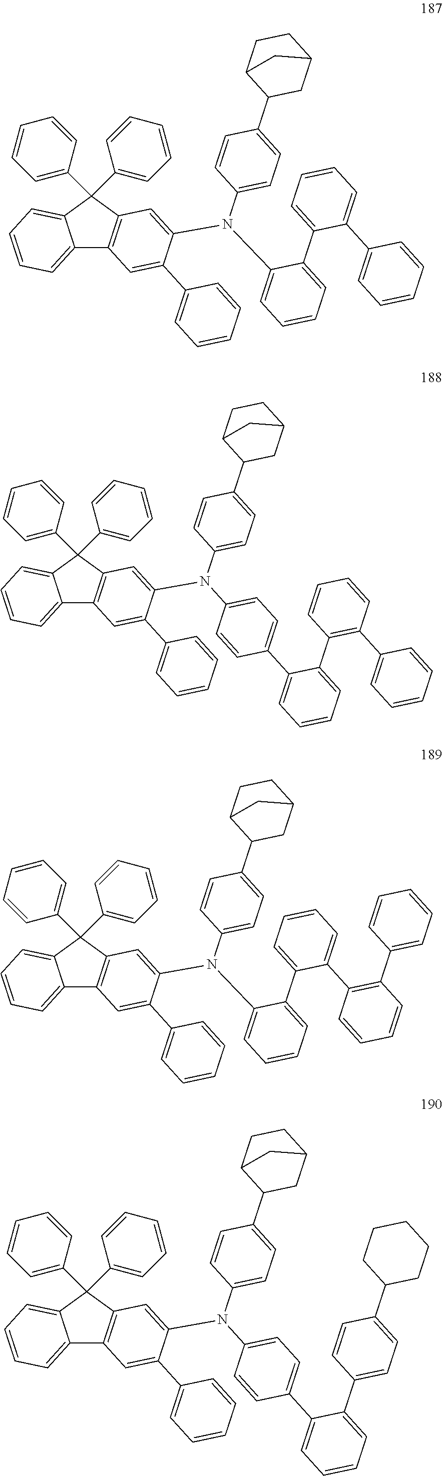 [SMILES: c1ccc(-c2ccccc2-c2ccccc2N(c2ccc(C3CC4CCC3C4)cc2)c2cc3c(cc2-c2ccccc2)-c2ccccc2C3(c2ccccc2)c2ccccc2)cc1 ; c1ccc(-c2ccccc2-c2ccccc2-c2ccc(N(c3ccc(C4CC5CCC4C5)cc3)c3cc4c(cc3-c3ccccc3)-c3ccccc3C4(c3ccccc3)c3ccccc3)cc2)cc1 ; c1ccc(-c2ccccc2-c2ccccc2-c2ccccc2N(c2ccc(C3CC4CCC3C4)cc2)c2cc3c(cc2-c2ccccc2)-c2ccccc2C3(c2ccccc2)c2ccccc2)cc1 ; c1ccc(-c2cc3c(cc2N(c2ccc(-c4ccccc4-c4ccc(C5CCCCC5)cc4)cc2)c2ccc(C4CC5CCC4C5)cc2)C(c2ccccc2)(c2ccccc2)c2ccccc2-3)cc1]

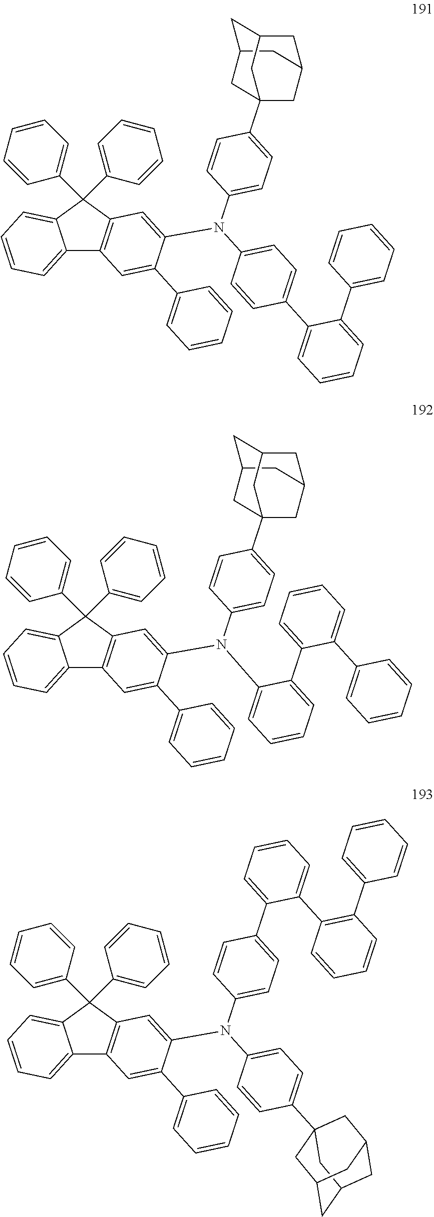 [SMILES: c1ccc(-c2ccccc2-c2ccc(N(c3ccc(C45CC6CC(CC(C6)C4)C5)cc3)c3cc4c(cc3-c3ccccc3)-c3ccccc3C4(c3ccccc3)c3ccccc3)cc2)cc1 ; c1ccc(-c2ccccc2-c2ccccc2N(c2ccc(C34CC5CC(CC(C5)C3)C4)cc2)c2cc3c(cc2-c2ccccc2)-c2ccccc2C3(c2ccccc2)c2ccccc2)cc1 ; c1ccc(-c2ccccc2-c2ccccc2-c2ccc(N(c3ccc(C45CC6CC(CC(C6)C4)C5)cc3)c3cc4c(cc3-c3ccccc3)-c3ccccc3C4(c3ccccc3)c3ccccc3)cc2)cc1]

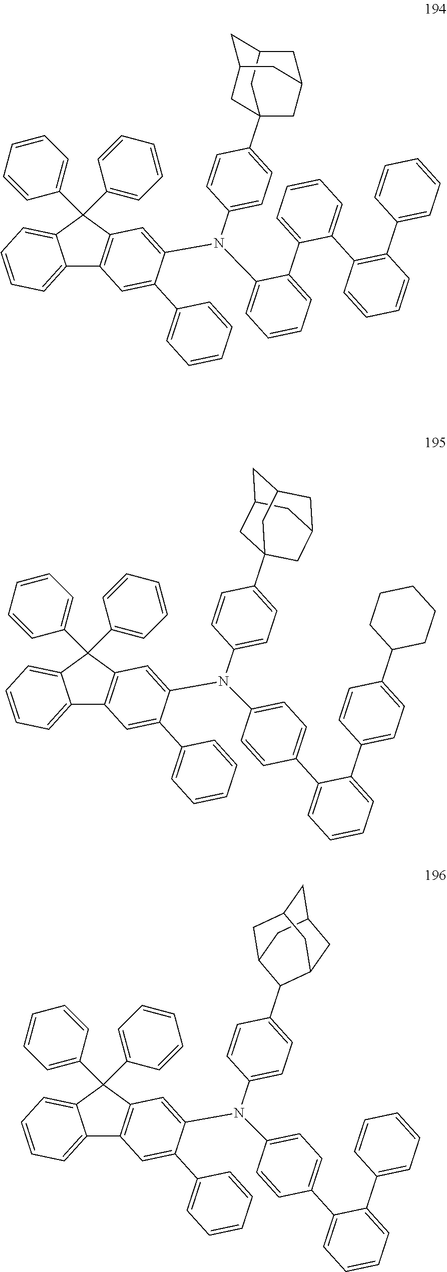 [SMILES: c1ccc(-c2ccccc2-c2ccccc2-c2ccccc2N(c2ccc(C34CC5CC(CC(C5)C3)C4)cc2)c2cc3c(cc2-c2ccccc2)-c2ccccc2C3(c2ccccc2)c2ccccc2)cc1 ; c1ccc(-c2cc3c(cc2N(c2ccc(-c4ccccc4-c4ccc(C5CCCCC5)cc4)cc2)c2ccc(C45CC6CC(CC(C6)C4)C5)cc2)C(c2ccccc2)(c2ccccc2)c2ccccc2-3)cc1 ; c1ccc(-c2ccccc2-c2ccc(N(c3ccc(C4C5CC6CC(C5)CC4C6)cc3)c3cc4c(cc3-c3ccccc3)-c3ccccc3C4(c3ccccc3)c3ccccc3)cc2)cc1]

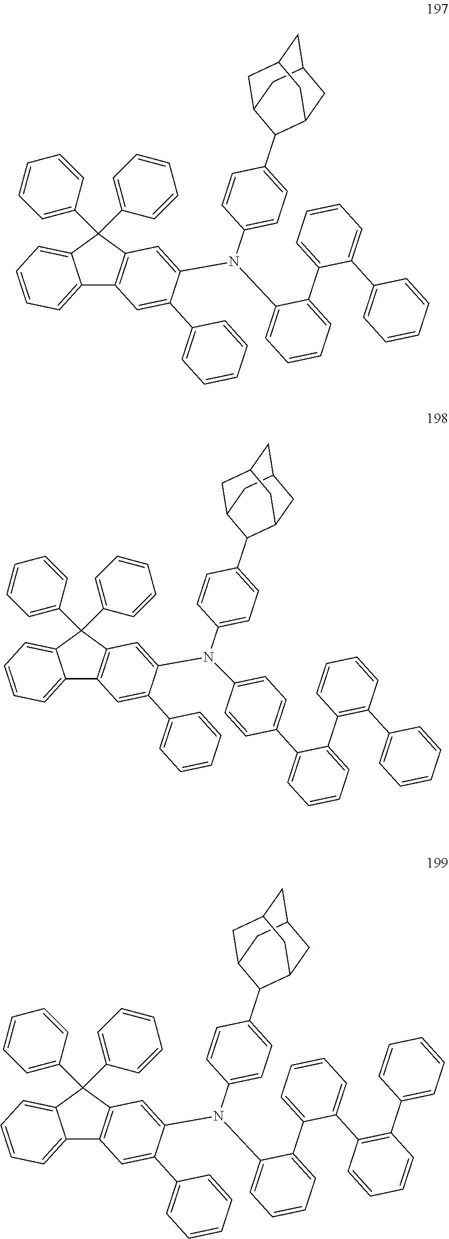 [SMILES: c1ccc(-c2ccccc2-c2ccccc2N(c2ccc(C3C4CC5CC(C4)CC3C5)cc2)c2cc3c(cc2-c2ccccc2)-c2ccccc2C3(c2ccccc2)c2ccccc2)cc1 ; c1ccc(-c2ccccc2-c2ccccc2-c2ccc(N(c3ccc(C4C5CC6CC(C5)CC4C6)cc3)c3cc4c(cc3-c3ccccc3)-c3ccccc3C4(c3ccccc3)c3ccccc3)cc2)cc1 ; c1ccc(-c2ccccc2-c2ccccc2-c2ccccc2N(c2ccc(C3C4CC5CC(C4)CC3C5)cc2)c2cc3c(cc2-c2ccccc2)-c2ccccc2C3(c2ccccc2)c2ccccc2)cc1]

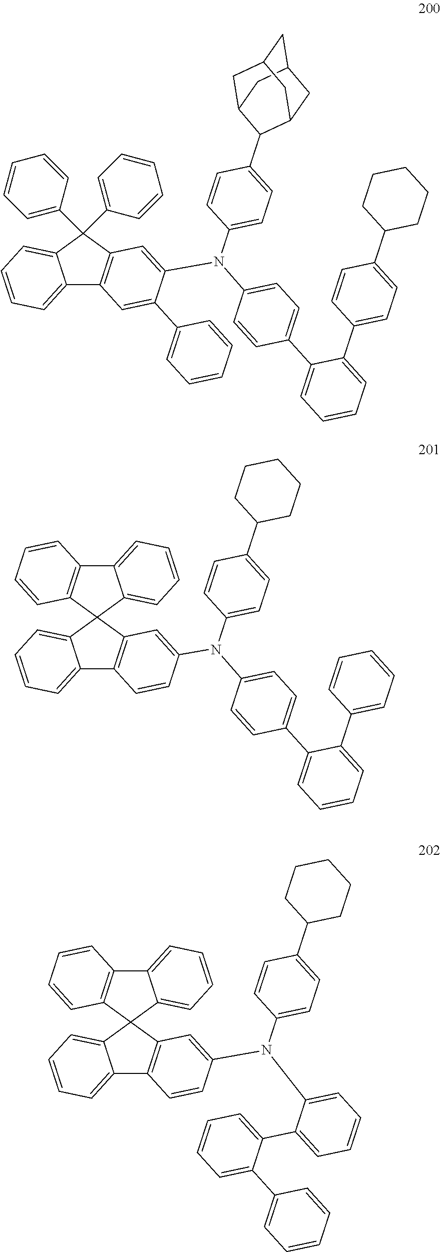 [SMILES: c1ccc(-c2cc3c(cc2N(c2ccc(-c4ccccc4-c4ccc(C5CCCCC5)cc4)cc2)c2ccc(C4C5CC6CC(C5)CC4C6)cc2)C(c2ccccc2)(c2ccccc2)c2ccccc2-3)cc1 ; c1ccc(-c2ccccc2-c2ccc(N(c3ccc(C4CCCCC4)cc3)c3ccc4c(c3)C3(c5ccccc5-c5ccccc53)c3ccccc3-4)cc2)cc1 ; c1ccc(-c2ccccc2-c2ccccc2N(c2ccc(C3CCCCC3)cc2)c2ccc3c(c2)C2(c4ccccc4-c4ccccc42)c2ccccc2-3)cc1]

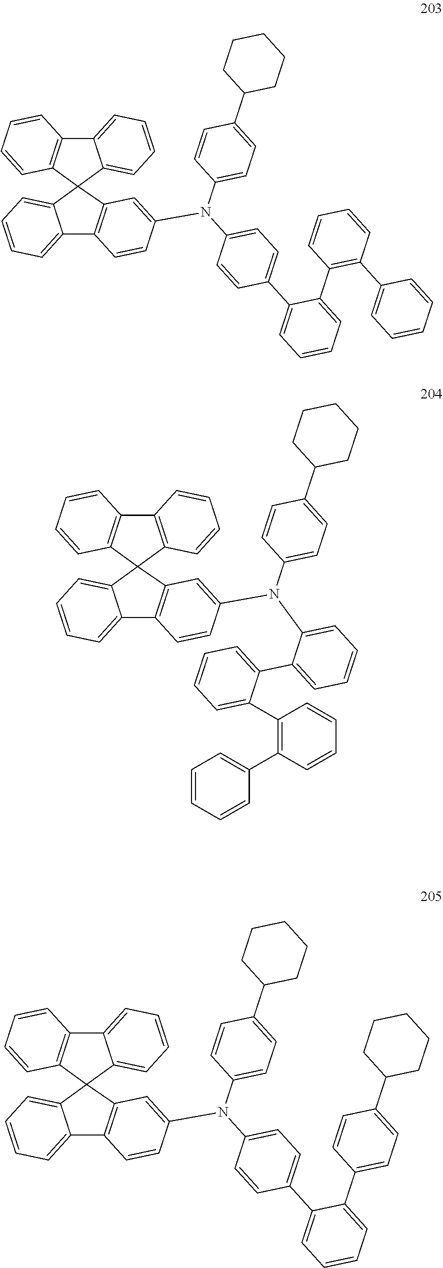 [SMILES: c1ccc(-c2ccccc2-c2ccccc2-c2ccc(N(c3ccc(C4CCCCC4)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(C3CCCCC3)cc2)c2ccc3c(c2)C2(c4ccccc4-c4ccccc42)c2ccccc2-3)cc1 ; c1ccc(-c2ccc(N(c3ccc(C4CCCCC4)cc3)c3ccc4c(c3)C3(c5ccccc5-c5ccccc53)c3ccccc3-4)cc2)c(-c2ccc(C3CCCCC3)cc2)c1]

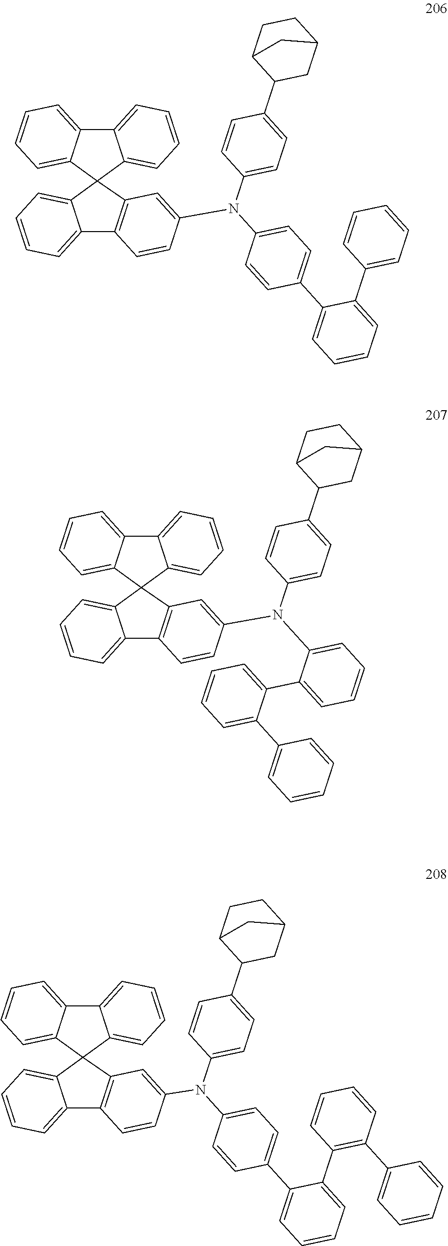 [SMILES: c1ccc(-c2ccccc2-c2ccc(N(c3ccc(C4CC5CCC4C5)cc3)c3ccc4c(c3)C3(c5ccccc5-c5ccccc53)c3ccccc3-4)cc2)cc1 ; c1ccc(-c2ccccc2-c2ccccc2N(c2ccc(C3CC4CCC3C4)cc2)c2ccc3c(c2)C2(c4ccccc4-c4ccccc42)c2ccccc2-3)cc1 ; c1ccc(-c2ccccc2-c2ccccc2-c2ccc(N(c3ccc(C4CC5CCC4C5)cc3)c3ccc4c(c3)C3(c5ccccc5-c5ccccc53)c3ccccc3-4)cc2)cc1]

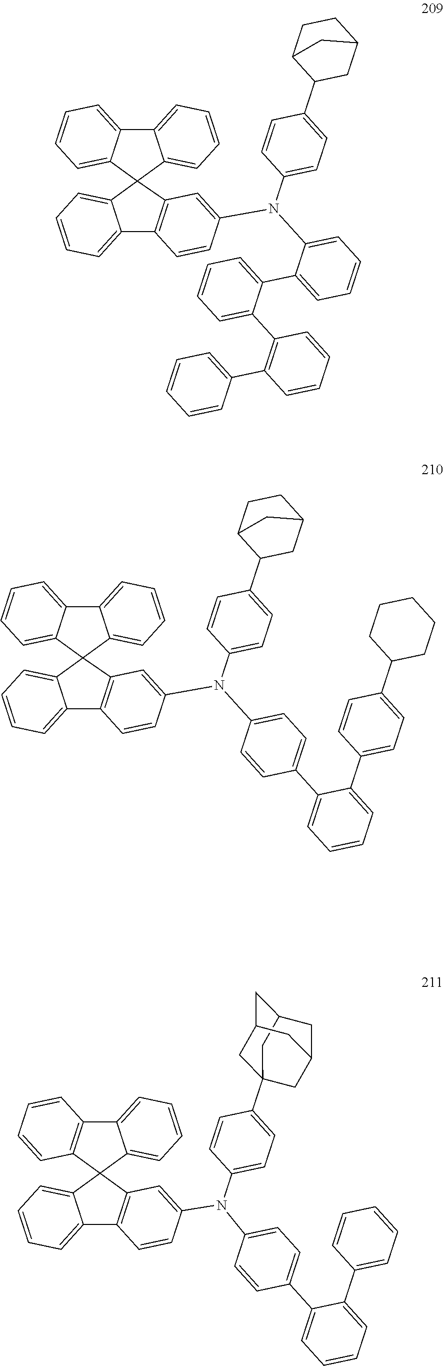 [SMILES: c1ccc(-c2ccccc2-c2ccccc2-c2ccccc2N(c2ccc(C3CC4CCC3C4)cc2)c2ccc3c(c2)C2(c4ccccc4-c4ccccc42)c2ccccc2-3)cc1 ; c1ccc(-c2ccc(N(c3ccc(C4CC5CCC4C5)cc3)c3ccc4c(c3)C3(c5ccccc5-c5ccccc53)c3ccccc3-4)cc2)c(-c2ccc(C3CCCCC3)cc2)c1 ; c1ccc(-c2ccccc2-c2ccc(N(c3ccc(C45CC6CC(CC(C6)C4)C5)cc3)c3ccc4c(c3)C3(c5ccccc5-c5ccccc53)c3ccccc3-4)cc2)cc1]

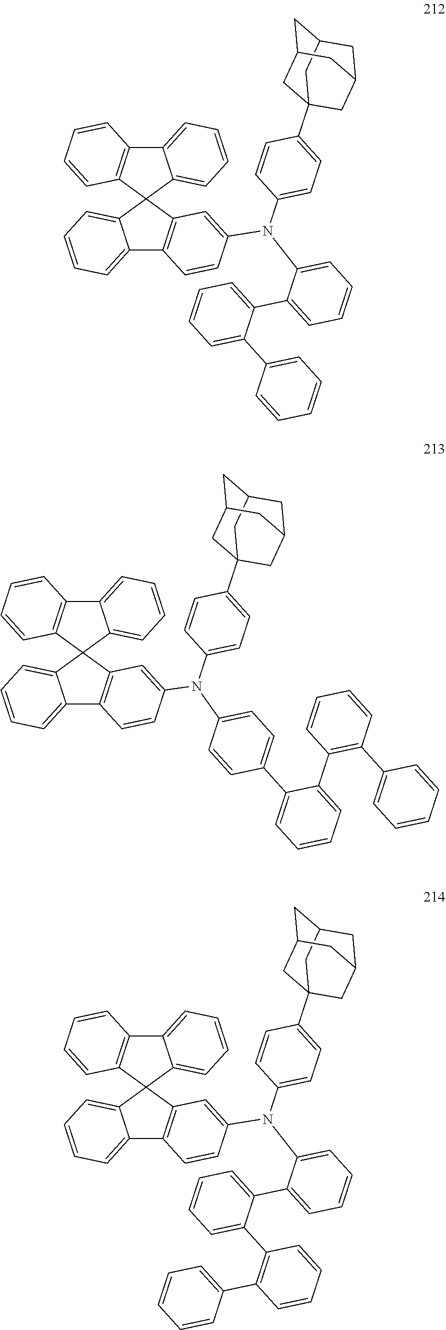 [SMILES: c1ccc(-c2ccccc2-c2ccccc2N(c2ccc(C34CC5CC(CC(C5)C3)C4)cc2)c2ccc3c(c2)C2(c4ccccc4-c4ccccc42)c2ccccc2-3)cc1 ; c1ccc(-c2ccccc2-c2ccccc2-c2ccc(N(c3ccc(C45CC6CC(CC(C6)C4)C5)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(C34CC5CC(CC(C5)C3)C4)cc2)c2ccc3c(c2)C2(c4ccccc4-c4ccccc42)c2ccccc2-3)cc1]

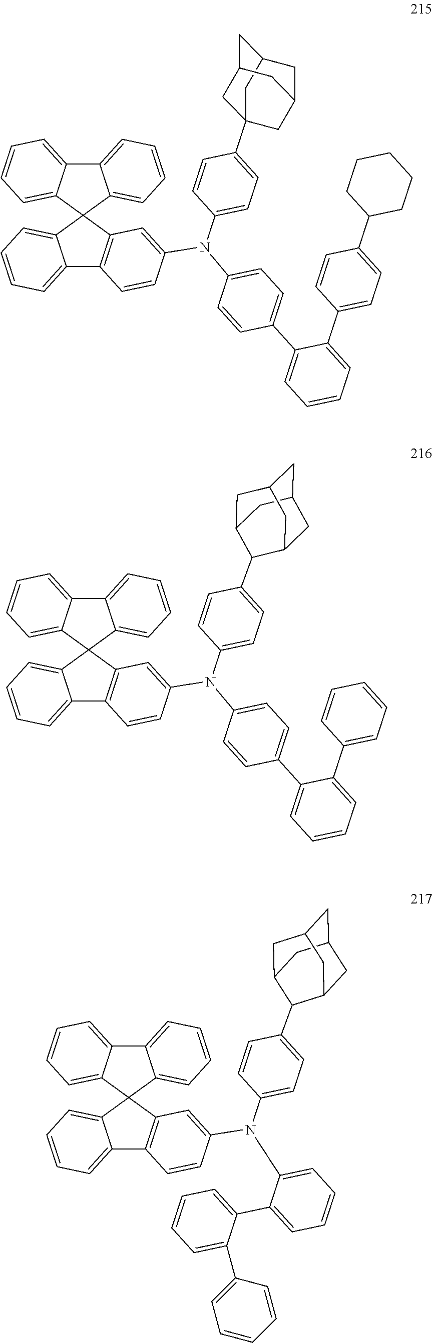 [SMILES: c1ccc(-c2ccc(N(c3ccc(C45CC6CC(CC(C6)C4)C5)cc3)c3ccc4c(c3)C3(c5ccccc5-c5ccccc53)c3ccccc3-4)cc2)c(-c2ccc(C3CCCCC3)cc2)c1 ; c1ccc(-c2ccccc2-c2ccc(N(c3ccc(C4C5CC6CC(C5)CC4C6)cc3)c3ccc4c(c3)C3(c5ccccc5-c5ccccc53)c3ccccc3-4)cc2)cc1 ; c1ccc(-c2ccccc2-c2ccccc2N(c2ccc(C3C4CC5CC(C4)CC3C5)cc2)c2ccc3c(c2)C2(c4ccccc4-c4ccccc42)c2ccccc2-3)cc1]

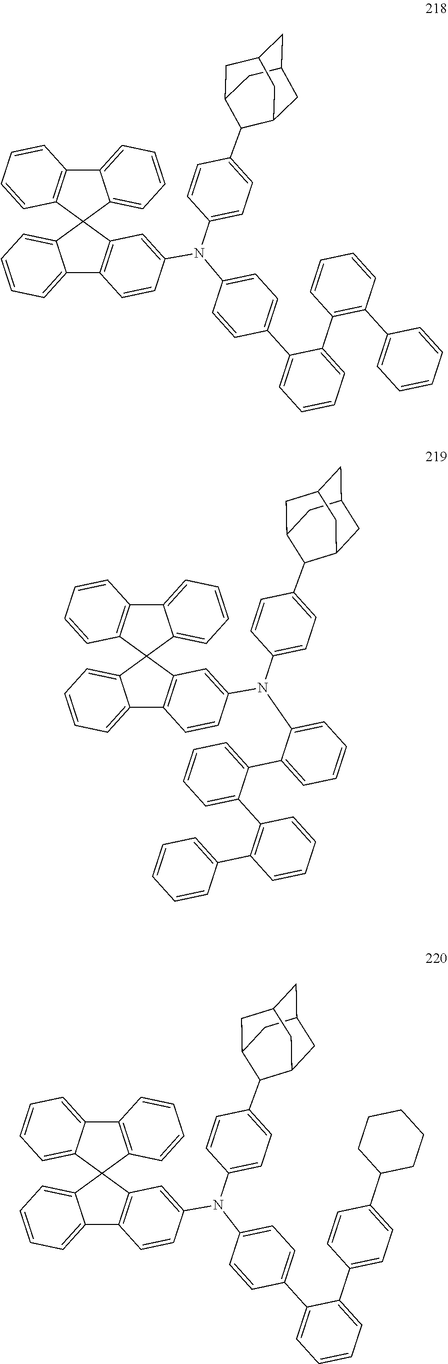 [SMILES: c1ccc(-c2ccccc2-c2ccccc2-c2ccc(N(c3ccc(C4C5CC6CC(C5)CC4C6)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(C3C4CC5CC(C4)CC3C5)cc2)c2ccc3c(c2)C2(c4ccccc4-c4ccccc42)c2ccccc2-3)cc1 ; c1ccc(-c2ccc(N(c3ccc(C4C5CC6CC(C5)CC4C6)cc3)c3ccc4c(c3)C3(c5ccccc5-c5ccccc53)c3ccccc3-4)cc2)c(-c2ccc(C3CCCCC3)cc2)c1]

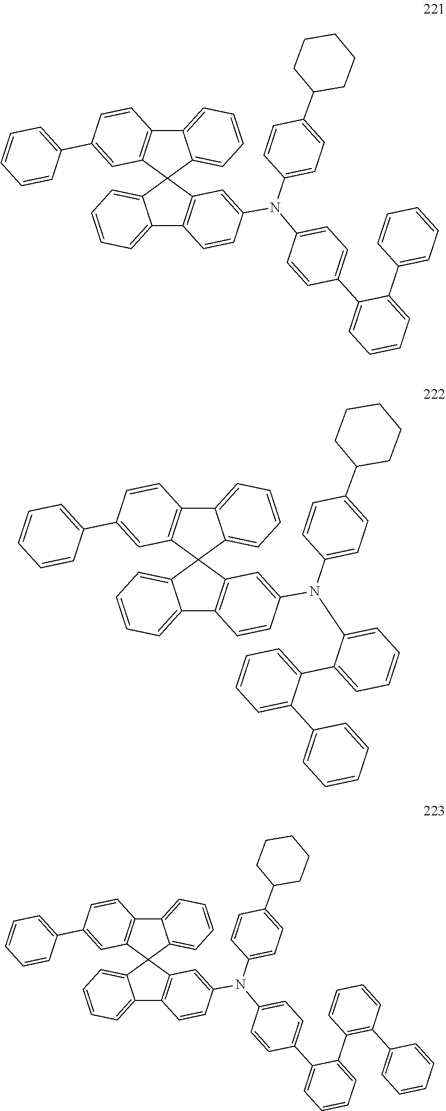 [SMILES: c1ccc(-c2ccc3c(c2)C2(c4ccccc4-3)c3ccccc3-c3ccc(N(c4ccc(-c5ccccc5-c5ccccc5)cc4)c4ccc(C5CCCCC5)cc4)cc32)cc1 ; c1ccc(-c2ccc3c(c2)C2(c4ccccc4-3)c3ccccc3-c3ccc(N(c4ccc(C5CCCCC5)cc4)c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccccc5-c5ccccc5)cc4)c4ccc(C5CCCCC5)cc4)cc32)cc1]

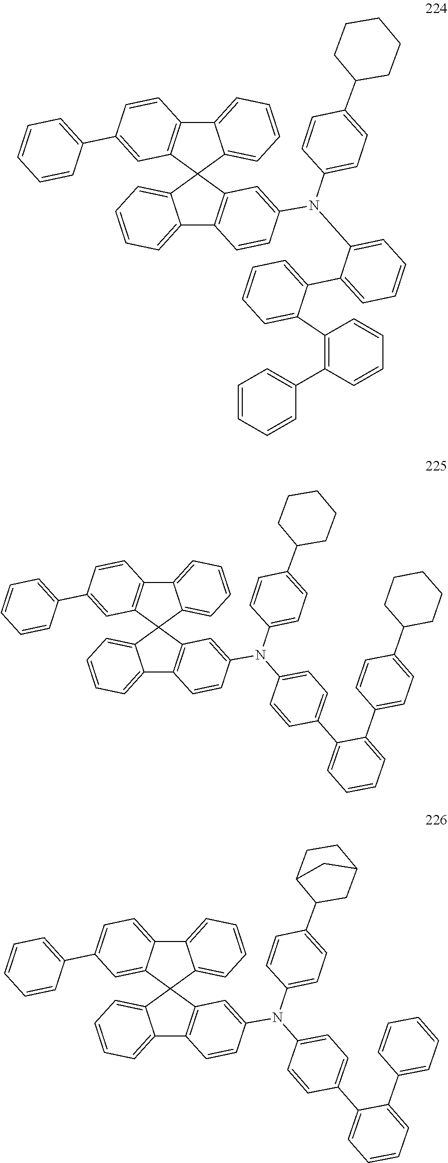 [SMILES: c1ccc(-c2ccc3c(c2)C2(c4ccccc4-3)c3ccccc3-c3ccc(N(c4ccc(C5CCCCC5)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccc(C6CCCCC6)cc5)cc4)c4ccc(C5CCCCC5)cc4)cc32)cc1 ; c1ccc(-c2ccc3c(c2)C2(c4ccccc4-3)c3ccccc3-c3ccc(N(c4ccc(-c5ccccc5-c5ccccc5)cc4)c4ccc(C5CC6CCC5C6)cc4)cc32)cc1]

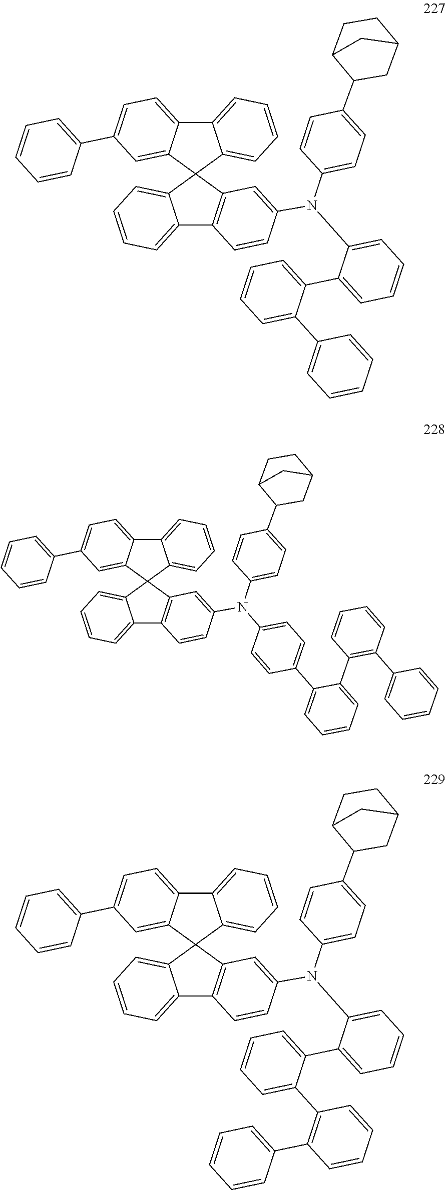 [SMILES: c1ccc(-c2ccc3c(c2)C2(c4ccccc4-3)c3ccccc3-c3ccc(N(c4ccc(C5CC6CCC5C6)cc4)c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccccc5-c5ccccc5)cc4)c4ccc(C5CC6CCC5C6)cc4)cc32)cc1 ; c1ccc(-c2ccc3c(c2)C2(c4ccccc4-3)c3ccccc3-c3ccc(N(c4ccc(C5CC6CCC5C6)cc4)c4ccccc4-c4ccccc4-c4ccccc4-c4ccccc4)cc32)cc1]

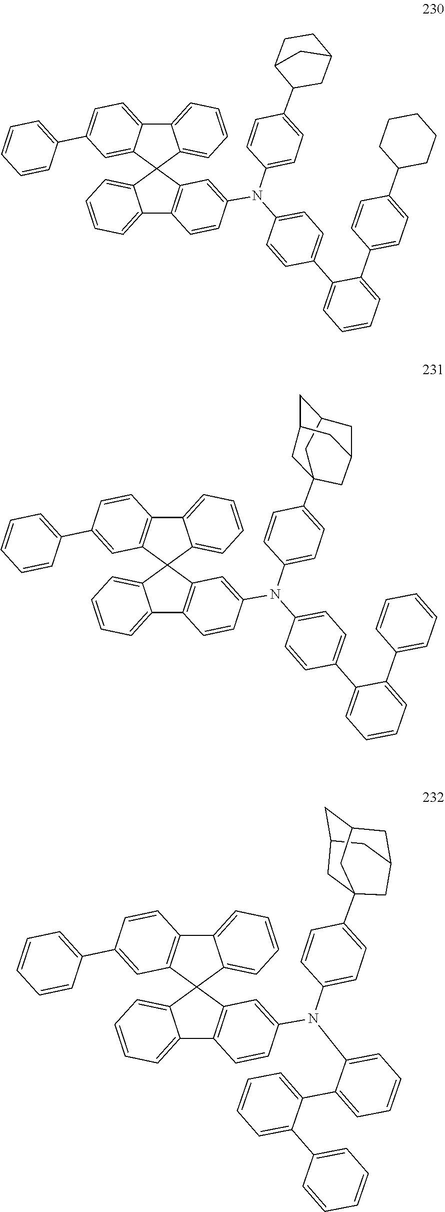 [SMILES: c1ccc(-c2ccc3c(c2)C2(c4ccccc4-3)c3ccccc3-c3ccc(N(c4ccc(-c5ccccc5-c5ccc(C6CCCCC6)cc5)cc4)c4ccc(C5CC6CCC5C6)cc4)cc32)cc1 ; c1ccc(-c2ccc3c(c2)C2(c4ccccc4-3)c3ccccc3-c3ccc(N(c4ccc(-c5ccccc5-c5ccccc5)cc4)c4ccc(C56CC7CC(CC(C7)C5)C6)cc4)cc32)cc1 ; c1ccc(-c2ccc3c(c2)C2(c4ccccc4-3)c3ccccc3-c3ccc(N(c4ccc(C56CC7CC(CC(C7)C5)C6)cc4)c4ccccc4-c4ccccc4-c4ccccc4)cc32)cc1]

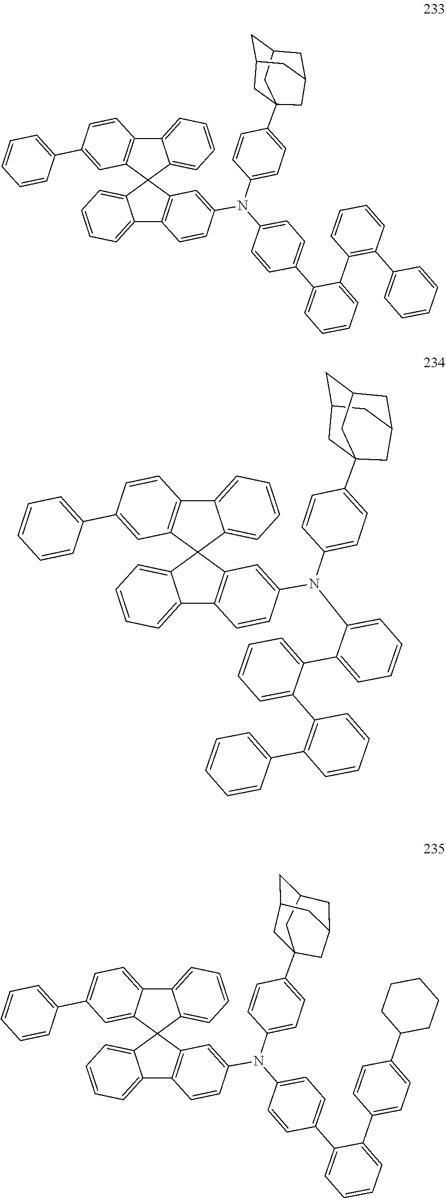

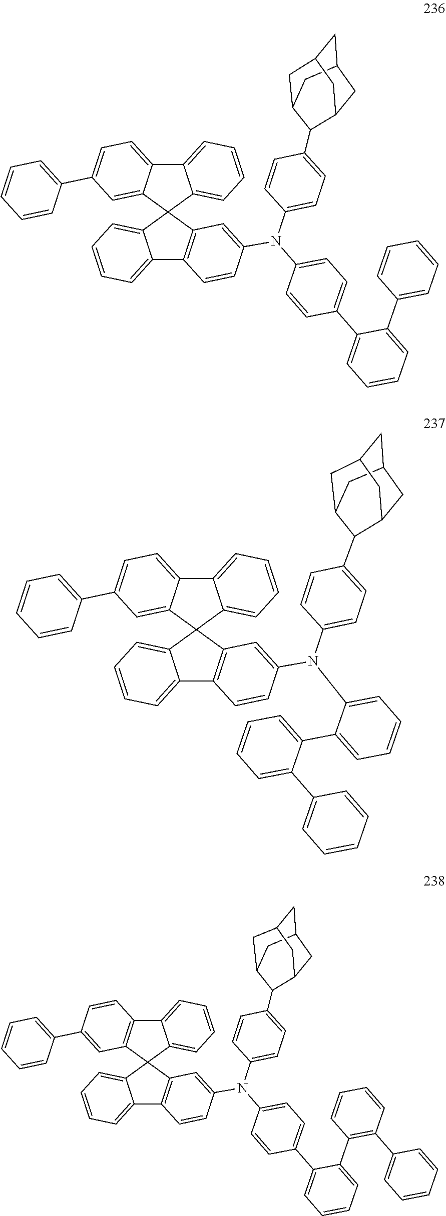 [SMILES: c1ccc(-c2ccc3c(c2)C2(c4ccccc4-3)c3ccccc3-c3ccc(N(c4ccc(-c5ccccc5-c5ccccc5)cc4)c4ccc(C5C6CC7CC(C6)CC5C7)cc4)cc32)cc1 ; c1ccc(-c2ccc3c(c2)C2(c4ccccc4-3)c3ccccc3-c3ccc(N(c4ccc(C5C6CC7CC(C6)CC5C7)cc4)c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccccc5-c5ccccc5)cc4)c4ccc(C5C6CC7CC(C6)CC5C7)cc4)cc32)cc1]

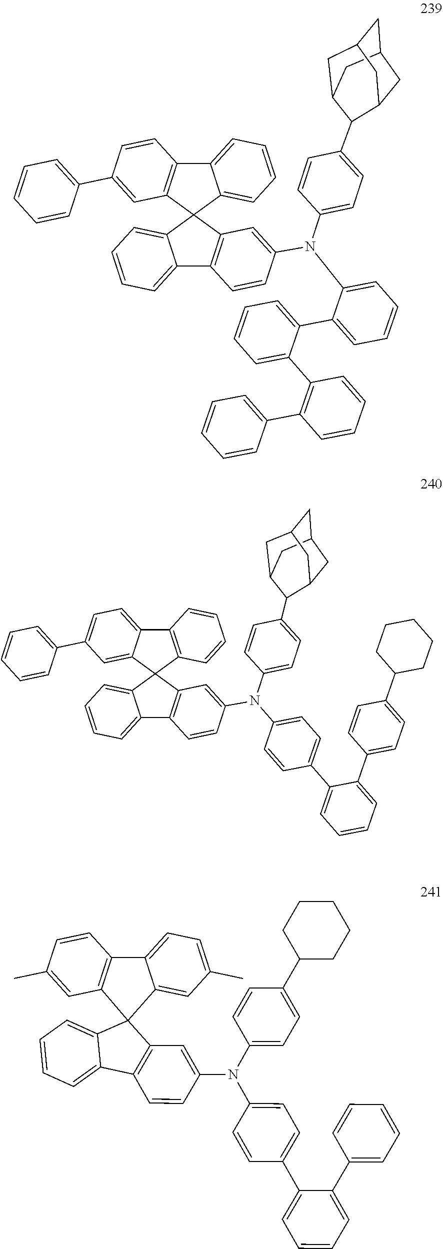 [SMILES: c1ccc(-c2ccc3c(c2)C2(c4ccccc4-3)c3ccccc3-c3ccc(N(c4ccc(C5C6CC7CC(C6)CC5C7)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccc(C6CCCCC6)cc5)cc4)c4ccc(C5C6CC7CC(C6)CC5C7)cc4)cc32)cc1 ; Cc1ccc2c(c1)C1(c3ccccc3-c3ccc(N(c4ccc(-c5ccccc5-c5ccccc5)cc4)c4ccc(C5CCCCC5)cc4)cc31)c1cc(C)ccc1-2]

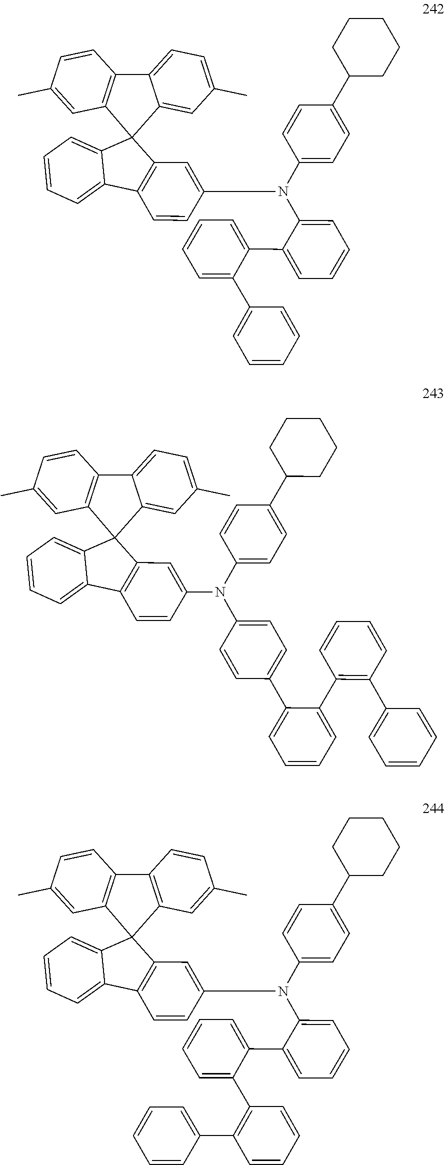 [SMILES: Cc1ccc2c(c1)C1(c3ccccc3-c3ccc(N(c4ccc(C5CCCCC5)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccccc5-c5ccccc5)cc4)c4ccc(C5CCCCC5)cc4)cc31)c1cc(C)ccc1-2 ; Cc1ccc2c(c1)C1(c3ccccc3-c3ccc(N(c4ccc(C5CCCCC5)cc4)c4ccccc4-c4ccccc4-c4ccccc4-c4ccccc4)cc31)c1cc(C)ccc1-2]

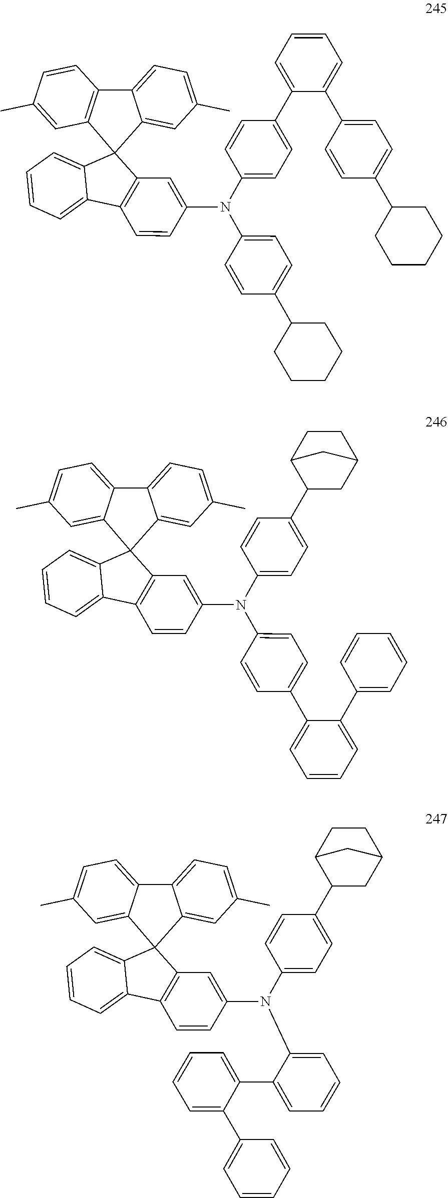 [SMILES: Cc1ccc2c(c1)C1(c3ccccc3-c3ccc(N(c4ccc(-c5ccccc5-c5ccc(C6CCCCC6)cc5)cc4)c4ccc(C5CCCCC5)cc4)cc31)c1cc(C)ccc1-2 ; Cc1ccc2c(c1)C1(c3ccccc3-c3ccc(N(c4ccc(-c5ccccc5-c5ccccc5)cc4)c4ccc(C5CC6CCC5C6)cc4)cc31)c1cc(C)ccc1-2 ; Cc1ccc2c(c1)C1(c3ccccc3-c3ccc(N(c4ccc(C5CC6CCC5C6)cc4)c4ccccc4-c4ccccc4-c4ccccc4)cc31)c1cc(C)ccc1-2]

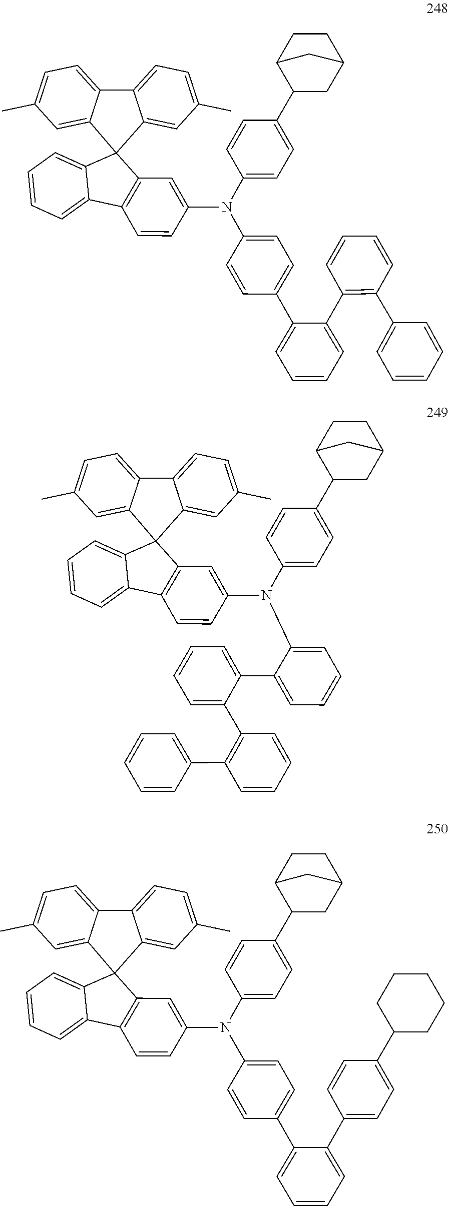

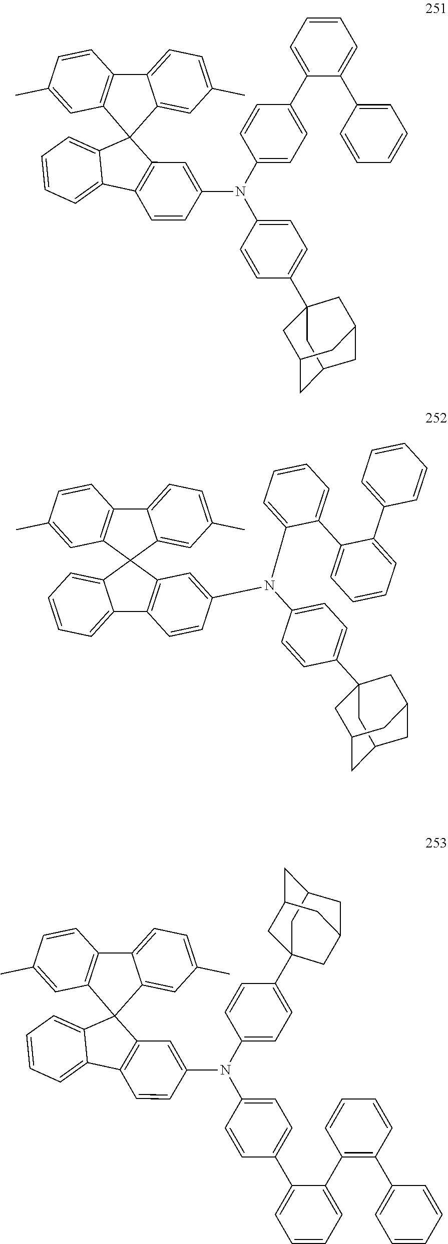 [SMILES: Cc1ccc2c(c1)C1(c3ccccc3-c3ccc(N(c4ccc(-c5ccccc5-c5ccccc5)cc4)c4ccc(C56CC7CC(CC(C7)C5)C6)cc4)cc31)c1cc(C)ccc1-2 ; Cc1ccc2c(c1)C1(c3ccccc3-c3ccc(N(c4ccc(C56CC7CC(CC(C7)C5)C6)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccccc5-c5ccccc5)cc4)c4ccc(C56CC7CC(CC(C7)C5)C6)cc4)cc31)c1cc(C)ccc1-2]

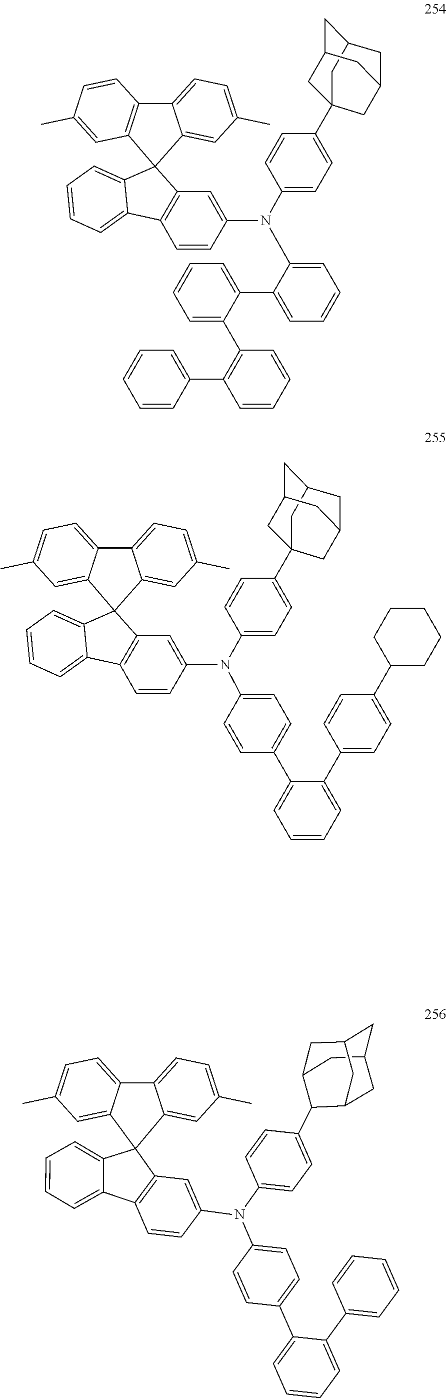 [SMILES: Cc1ccc2c(c1)C1(c3ccccc3-c3ccc(N(c4ccc(C56CC7CC(CC(C7)C5)C6)cc4)c4ccccc4-c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccc(C6CCCCC6)cc5)cc4)c4ccc(C56CC7CC(CC(C7)C5)C6)cc4)cc31)c1cc(C)ccc1-2 ; Cc1ccc2c(c1)C1(c3ccccc3-c3ccc(N(c4ccc(-c5ccccc5-c5ccccc5)cc4)c4ccc(C5C6CC7CC(C6)CC5C7)cc4)cc31)c1cc(C)ccc1-2]

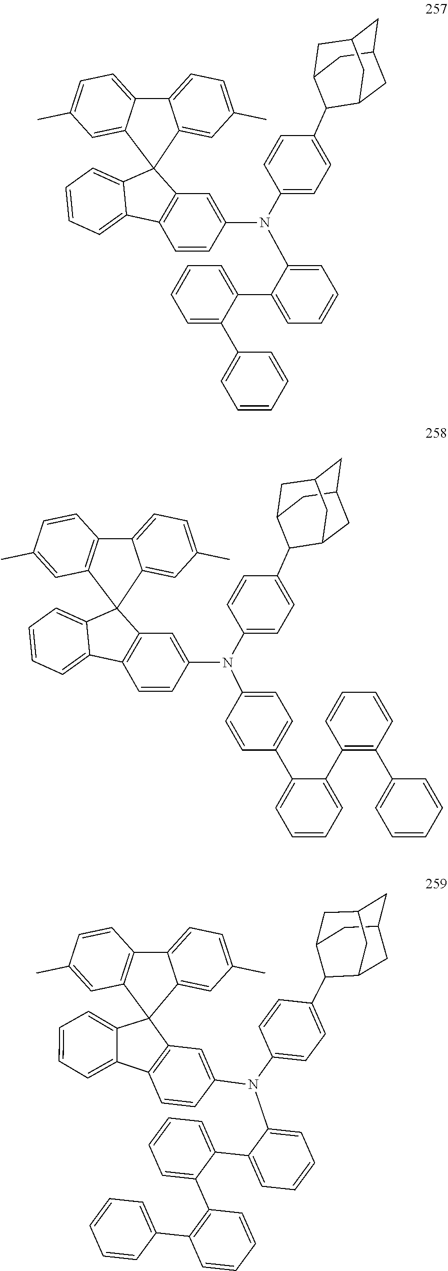 [SMILES: Cc1ccc2c(c1)C1(c3ccccc3-c3ccc(N(c4ccc(C5C6CC7CC(C6)CC5C7)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccccc5-c5ccccc5)cc4)c4ccc(C5C6CC7CC(C6)CC5C7)cc4)cc31)c1cc(C)ccc1-2 ; Cc1ccc2c(c1)C1(c3ccccc3-c3ccc(N(c4ccc(C5C6CC7CC(C6)CC5C7)cc4)c4ccccc4-c4ccccc4-c4ccccc4-c4ccccc4)cc31)c1cc(C)ccc1-2]

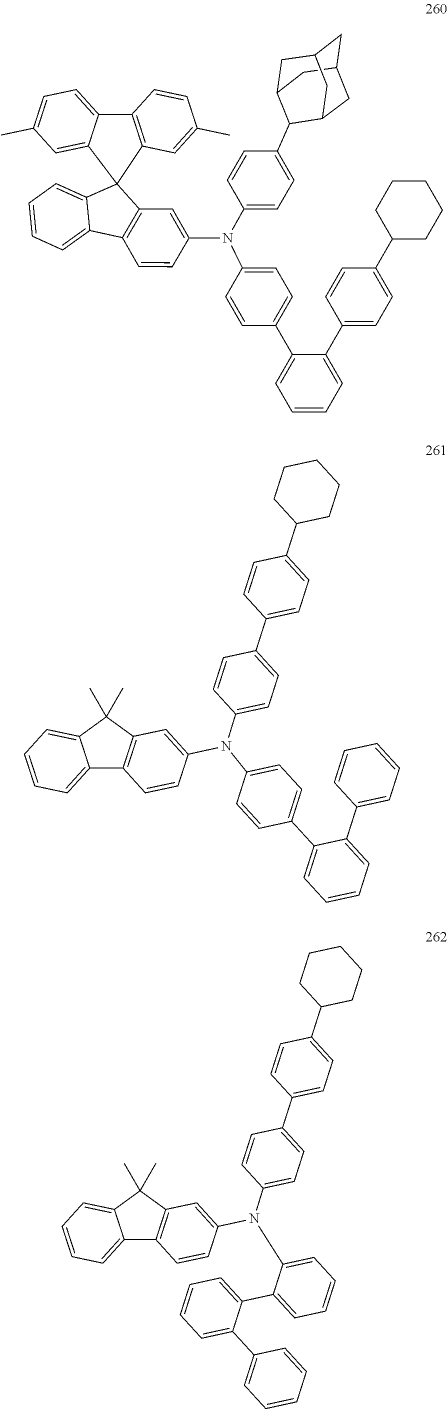

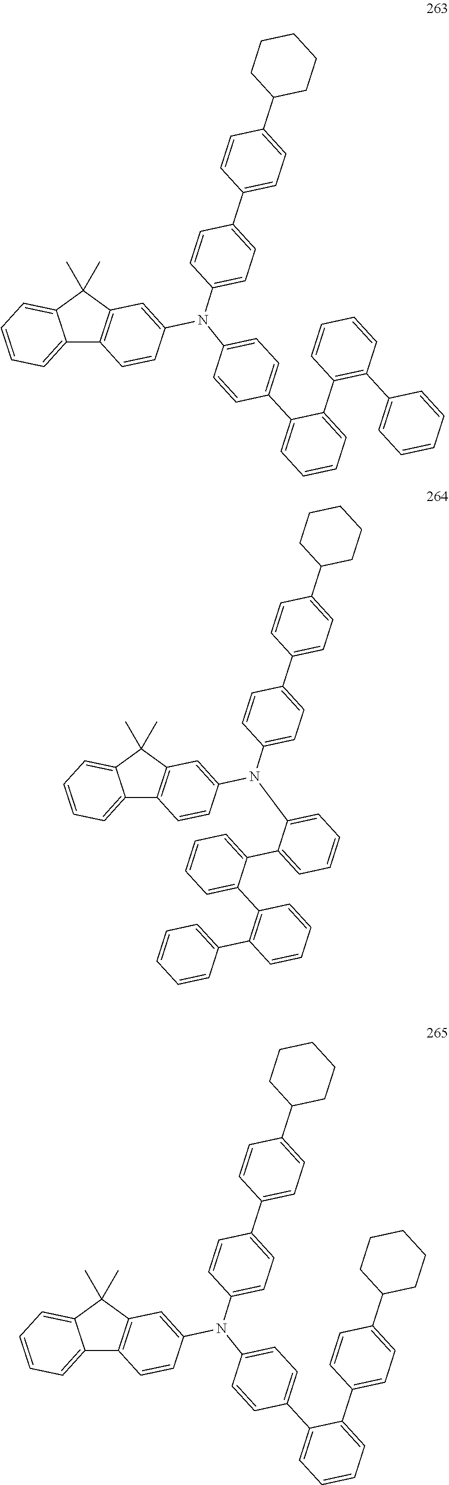

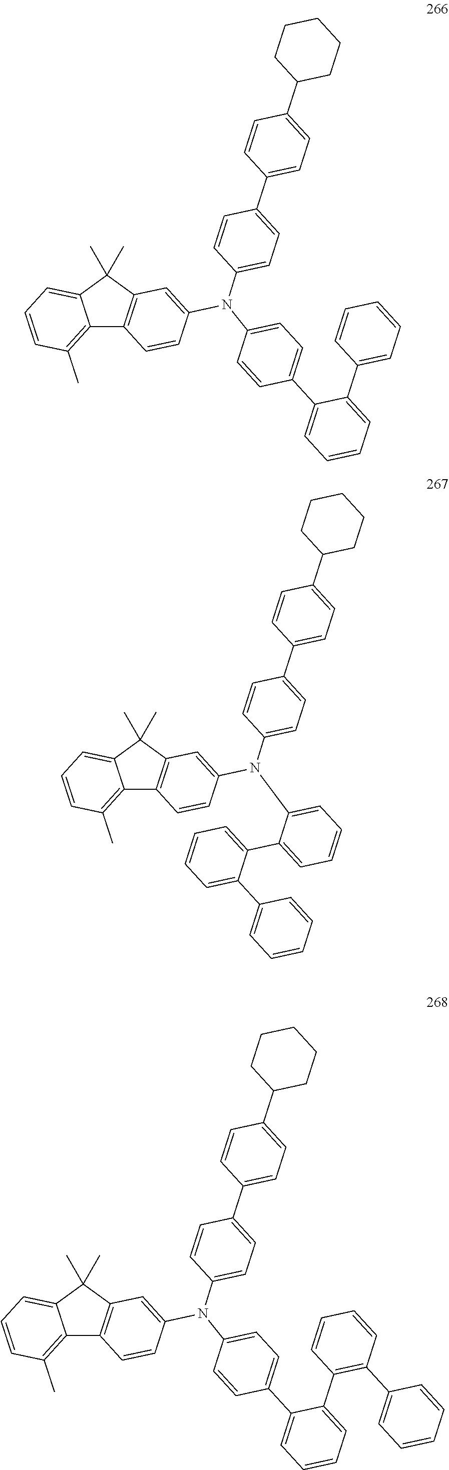

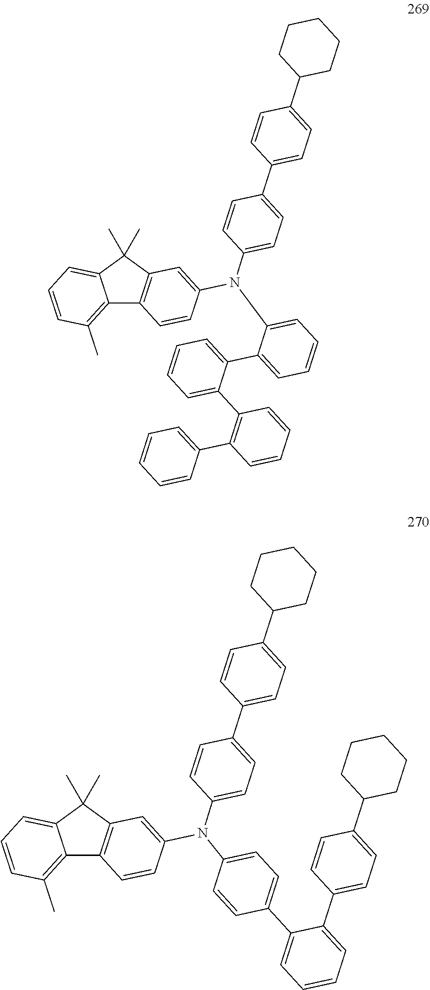 [SMILES: Cc1cccc2c1-c1ccc(N(c3ccc(-c4ccc(C5CCCCC5)cc4)cc3)c3ccccc3-c3ccccc3-c3ccccc3-c3ccccc3)cc1C2(C)C ; Cc1cccc2c1-c1ccc(N(c3ccc(-c4ccc(C5CCCCC5)cc4)cc3)c3ccc(-c4ccccc4-c4ccc(C5CCCCC5)cc4)cc3)cc1C2(C)C]

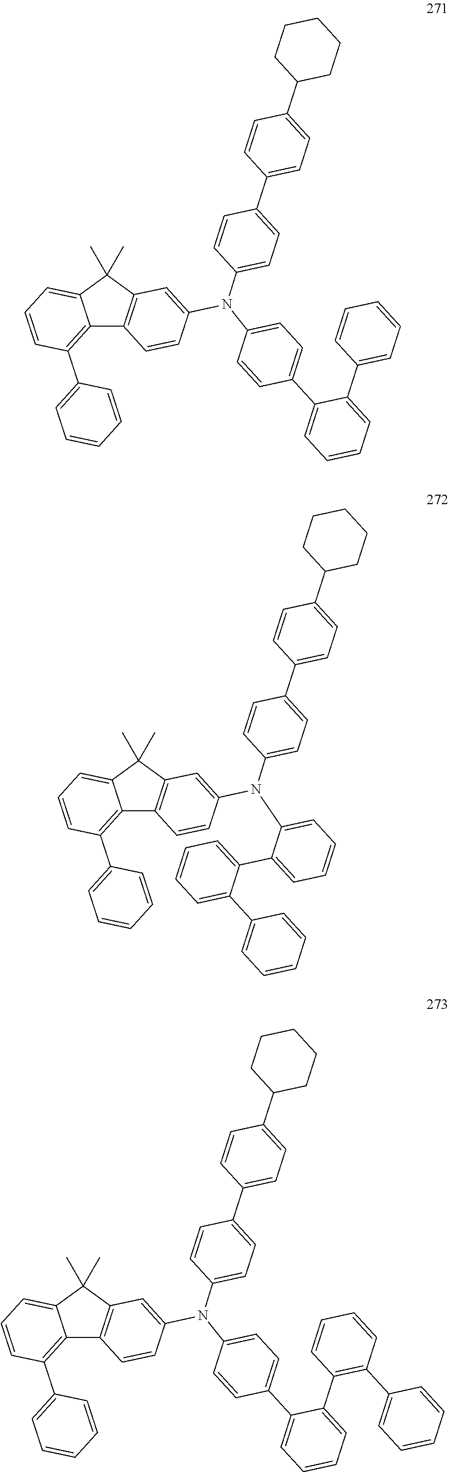 [SMILES: CC1(C)c2cc(N(c3ccc(-c4ccc(C5CCCCC5)cc4)cc3)c3ccc(-c4ccccc4-c4ccccc4)cc3)ccc2-c2c(-c3ccccc3)cccc21 ; CC1(C)c2cc(N(c3ccc(-c4ccc(C5CCCCC5)cc4)cc3)c3ccccc3-c3ccccc3-c3ccccc3)ccc2-c2c(-c3ccccc3)cccc21 ; CC1(C)c2cc(N(c3ccc(-c4ccc(C5CCCCC5)cc4)cc3)c3ccc(-c4ccccc4-c4ccccc4-c4ccccc4)cc3)ccc2-c2c(-c3ccccc3)cccc21]

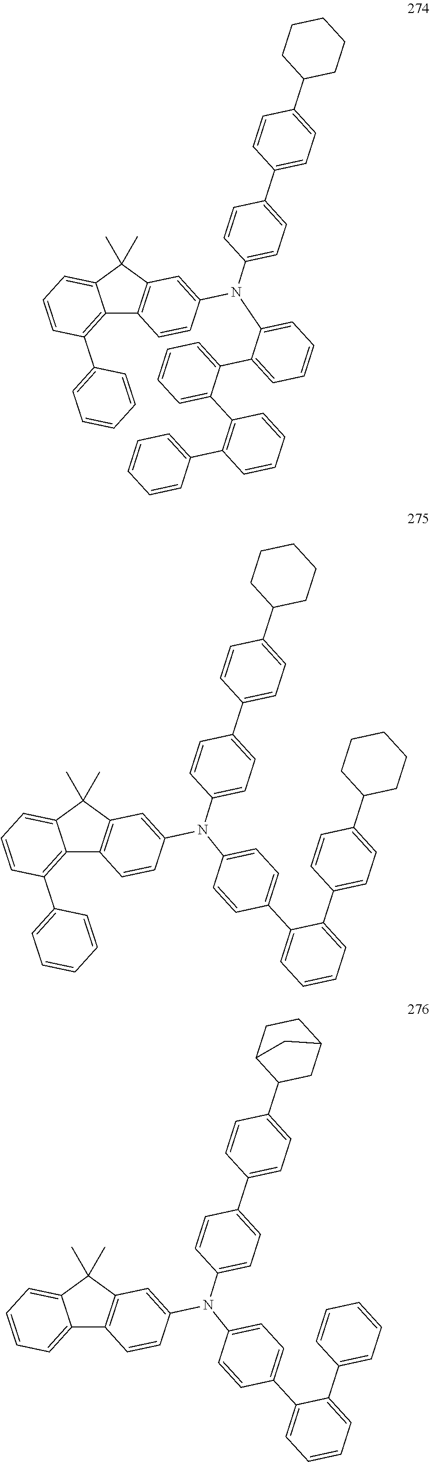

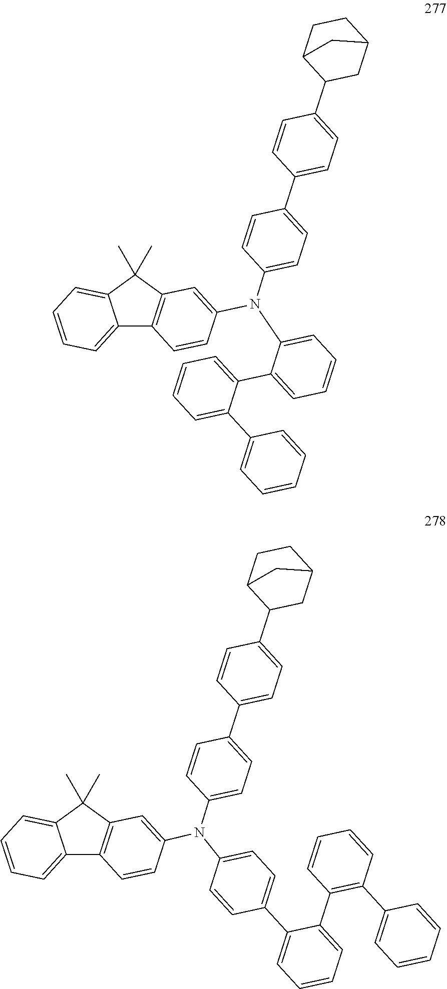 [SMILES: CC1(C)c2ccccc2-c2ccc(N(c3ccc(-c4ccc(C5CC6CCC5C6)cc4)cc3)c3ccccc3-c3ccccc3-c3ccccc3)cc21 ; CC1(C)c2ccccc2-c2ccc(N(c3ccc(-c4ccc(C5CC6CCC5C6)cc4)cc3)c3ccc(-c4ccccc4-c4ccccc4-c4ccccc4)cc3)cc21]

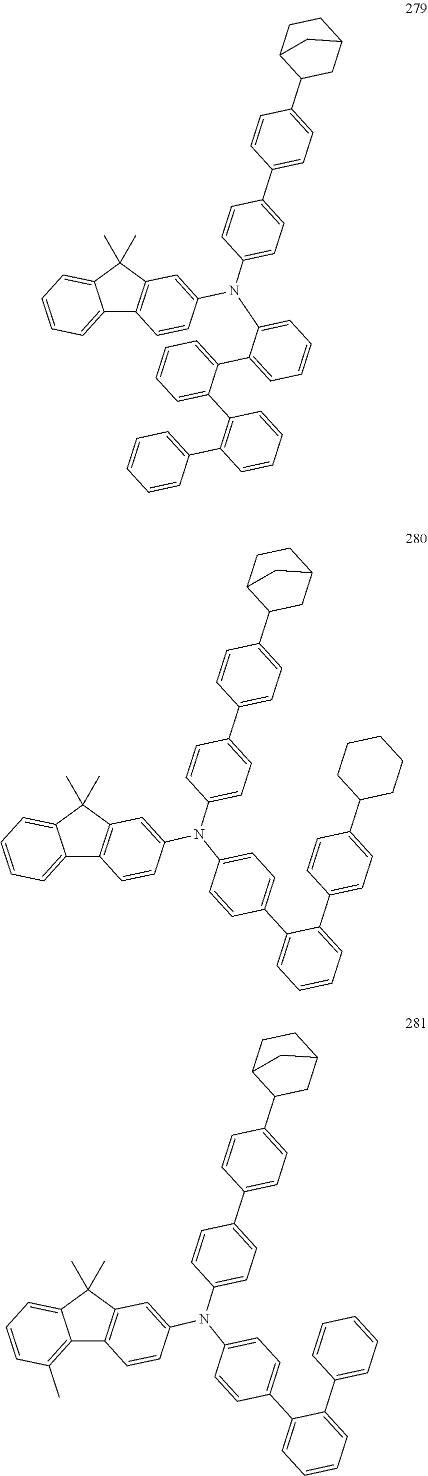

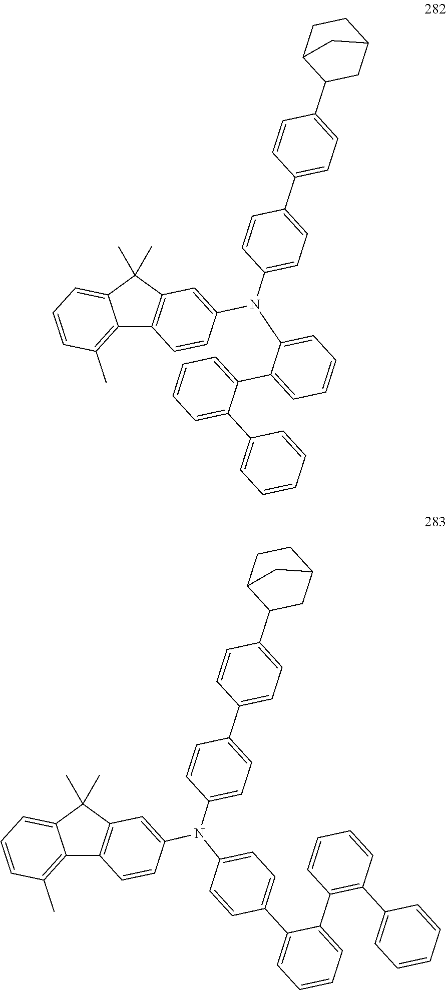 [SMILES: Cc1cccc2c1-c1ccc(N(c3ccc(-c4ccc(C5CC6CCC5C6)cc4)cc3)c3ccccc3-c3ccccc3-c3ccccc3)cc1C2(C)C ; Cc1cccc2c1-c1ccc(N(c3ccc(-c4ccc(C5CC6CCC5C6)cc4)cc3)c3ccc(-c4ccccc4-c4ccccc4-c4ccccc4)cc3)cc1C2(C)C]

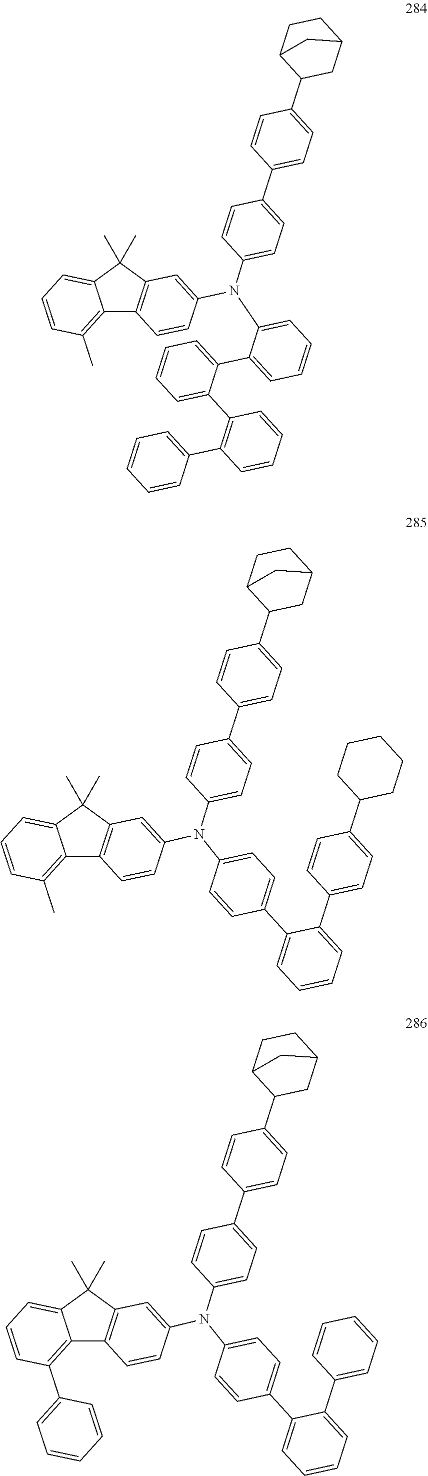

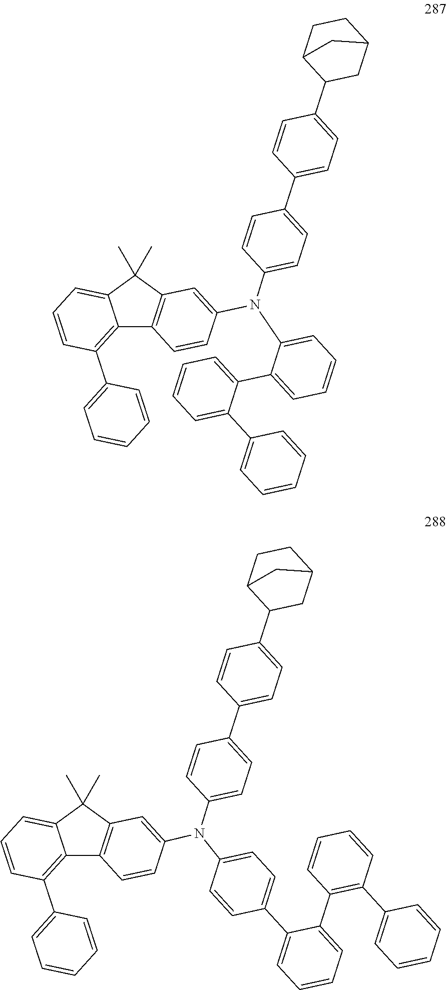 [SMILES: CC1(C)c2cc(N(c3ccc(-c4ccc(C5CC6CCC5C6)cc4)cc3)c3ccccc3-c3ccccc3-c3ccccc3)ccc2-c2c(-c3ccccc3)cccc21 ; CC1(C)c2cc(N(c3ccc(-c4ccc(C5CC6CCC5C6)cc4)cc3)c3ccc(-c4ccccc4-c4ccccc4-c4ccccc4)cc3)ccc2-c2c(-c3ccccc3)cccc21]

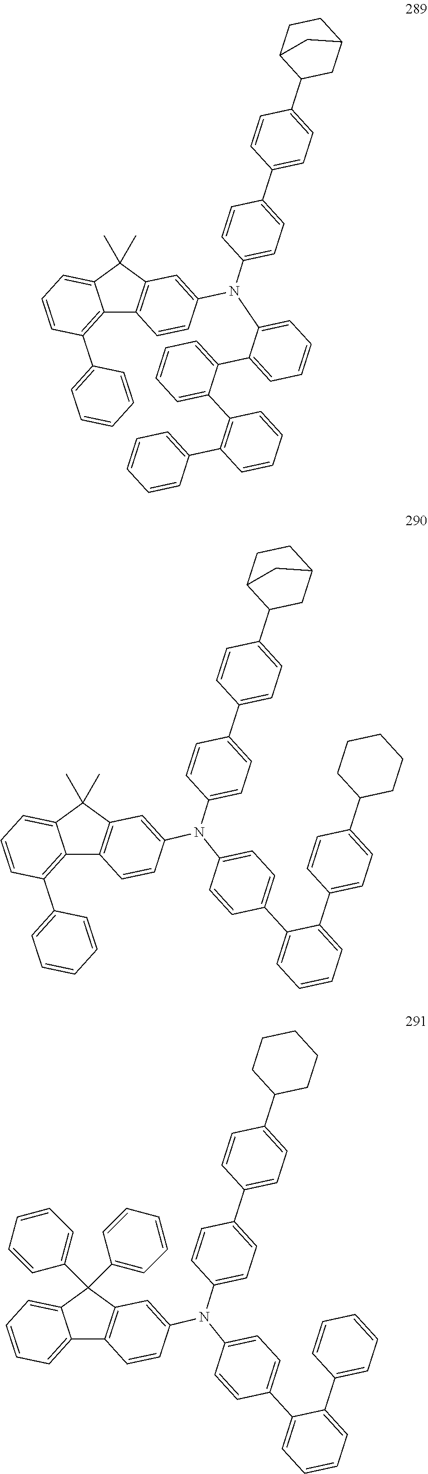

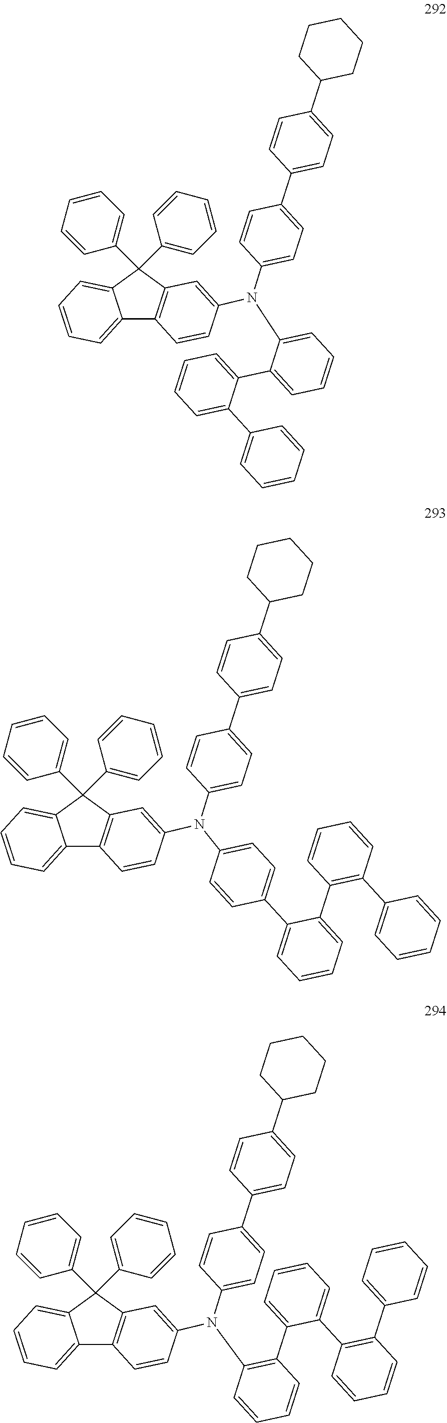 [SMILES: c1ccc(-c2ccccc2-c2ccccc2N(c2ccc(-c3ccc(C4CCCCC4)cc3)cc2)c2ccc3c(c2)C(c2ccccc2)(c2ccccc2)c2ccccc2-3)cc1 ; c1ccc(-c2ccccc2-c2ccccc2-c2ccc(N(c3ccc(-c4ccc(C5CCCCC5)cc4)cc3)c3ccc4c(c3)C(c3ccccc3)(c3ccccc3)c3ccccc3-4)cc2)cc1 ; c1ccc(-c2ccccc2-c2ccccc2-c2ccccc2N(c2ccc(-c3ccc(C4CCCCC4)cc3)cc2)c2ccc3c(c2)C(c2ccccc2)(c2ccccc2)c2ccccc2-3)cc1]

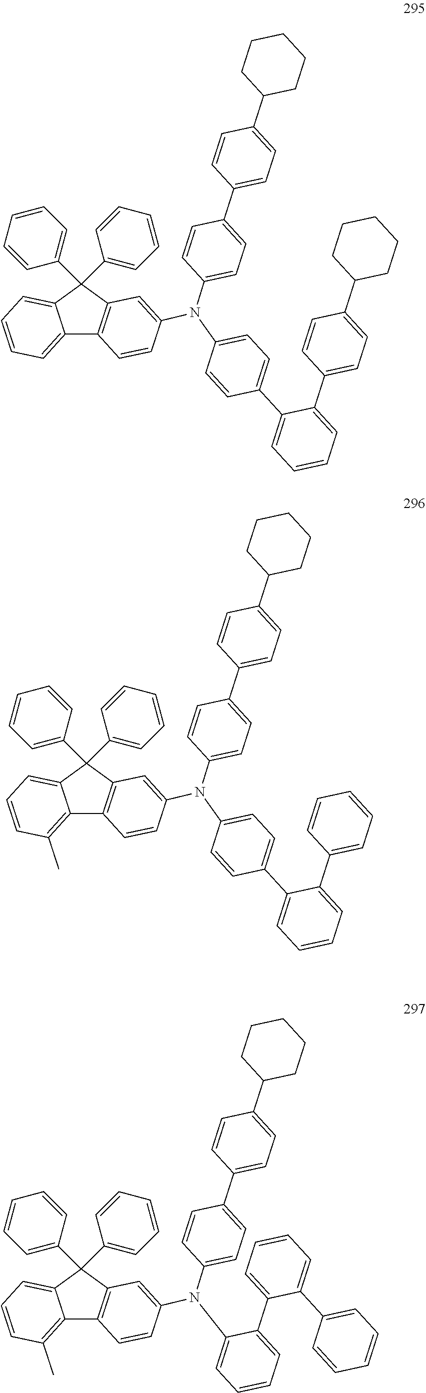

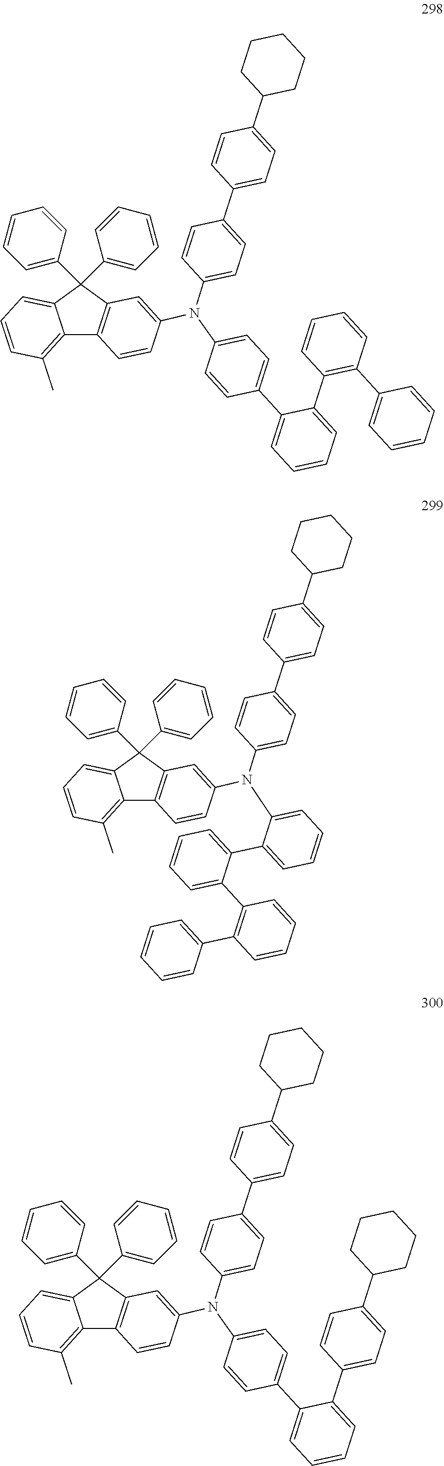 [SMILES: Cc1cccc2c1-c1ccc(N(c3ccc(-c4ccc(C5CCCCC5)cc4)cc3)c3ccc(-c4ccccc4-c4ccccc4-c4ccccc4)cc3)cc1C2(c1ccccc1)c1ccccc1 ; Cc1cccc2c1-c1ccc(N(c3ccc(-c4ccc(C5CCCCC5)cc4)cc3)c3ccccc3-c3ccccc3-c3ccccc3-c3ccccc3)cc1C2(c1ccccc1)c1ccccc1 ; Cc1cccc2c1-c1ccc(N(c3ccc(-c4ccc(C5CCCCC5)cc4)cc3)c3ccc(-c4ccccc4-c4ccc(C5CCCCC5)cc4)cc3)cc1C2(c1ccccc1)c1ccccc1]

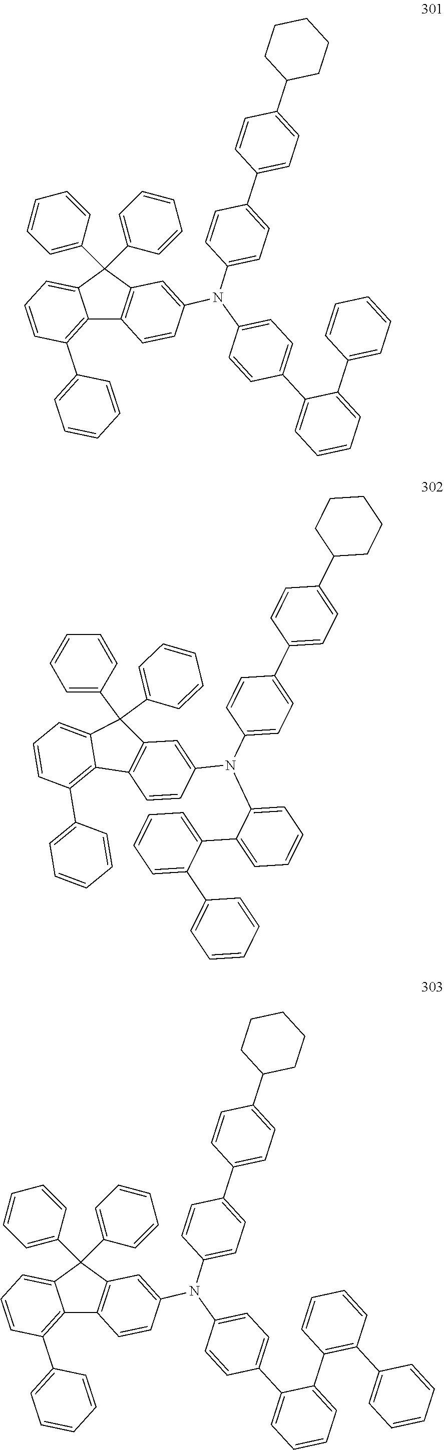

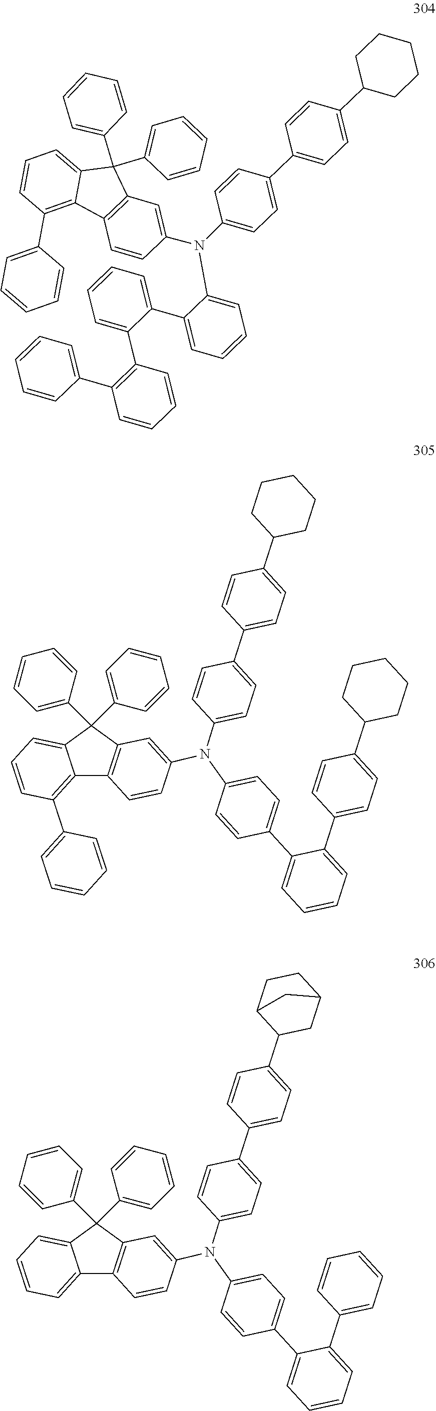 [SMILES: c1ccc(-c2ccccc2-c2ccccc2-c2ccccc2N(c2ccc(-c3ccc(C4CCCCC4)cc3)cc2)c2ccc3c(c2)C(c2ccccc2)(c2ccccc2)c2cccc(-c4ccccc4)c2-3)cc1 ; c1ccc(-c2cccc3c2-c2ccc(N(c4ccc(-c5ccc(C6CCCCC6)cc5)cc4)c4ccc(-c5ccccc5-c5ccc(C6CCCCC6)cc5)cc4)cc2C3(c2ccccc2)c2ccccc2)cc1 ; c1ccc(-c2ccccc2-c2ccc(N(c3ccc(-c4ccc(C5CC6CCC5C6)cc4)cc3)c3ccc4c(c3)C(c3ccccc3)(c3ccccc3)c3ccccc3-4)cc2)cc1]

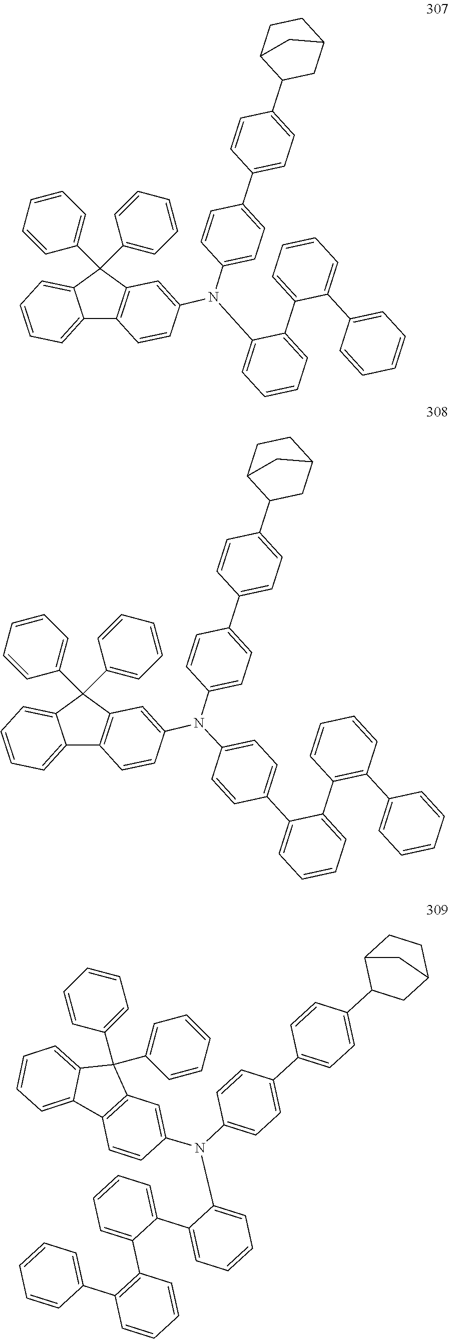 [SMILES: c1ccc(-c2ccccc2-c2ccccc2N(c2ccc(-c3ccc(C4CC5CCC4C5)cc3)cc2)c2ccc3c(c2)C(c2ccccc2)(c2ccccc2)c2ccccc2-3)cc1 ; c1ccc(-c2ccccc2-c2ccccc2-c2ccc(N(c3ccc(-c4ccc(C5CC6CCC5C6)cc4)cc3)c3ccc4c(c3)C(c3ccccc3)(c3ccccc3)c3ccccc3-4)cc2)cc1 ; c1ccc(-c2ccccc2-c2ccccc2-c2ccccc2N(c2ccc(-c3ccc(C4CC5CCC4C5)cc3)cc2)c2ccc3c(c2)C(c2ccccc2)(c2ccccc2)c2ccccc2-3)cc1]

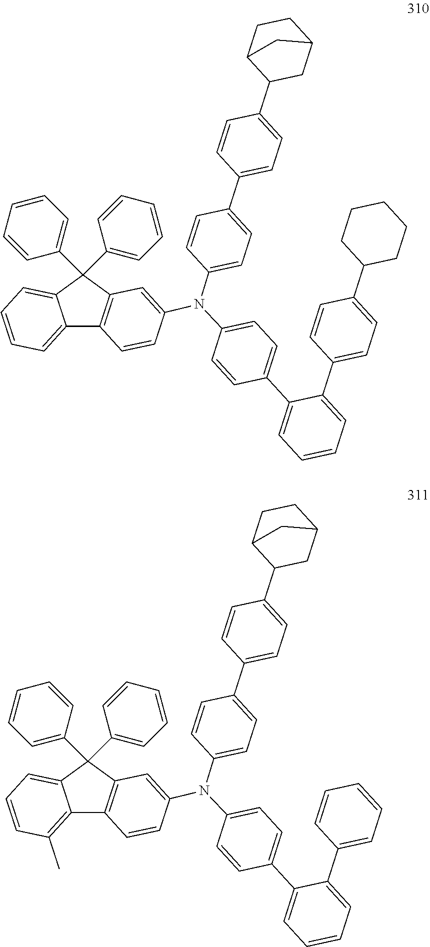

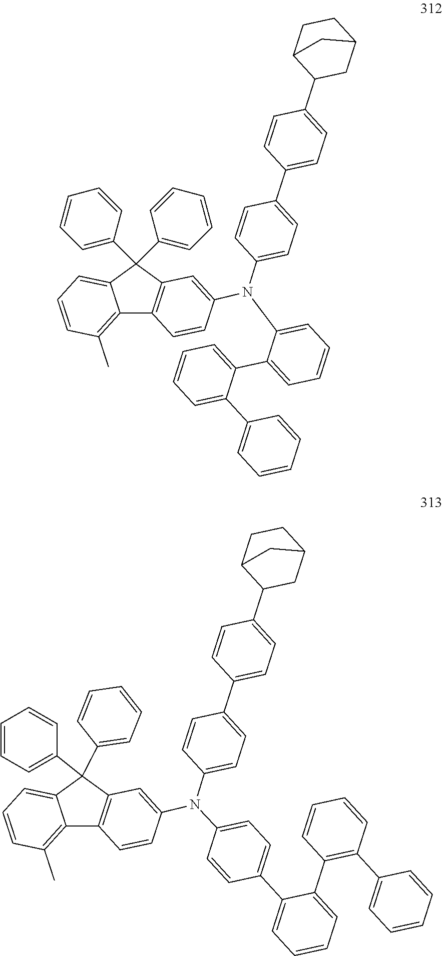 [SMILES: Cc1cccc2c1-c1ccc(N(c3ccc(-c4ccc(C5CC6CCC5C6)cc4)cc3)c3ccccc3-c3ccccc3-c3ccccc3)cc1C2(c1ccccc1)c1ccccc1 ; Cc1cccc2c1-c1ccc(N(c3ccc(-c4ccc(C5CC6CCC5C6)cc4)cc3)c3ccc(-c4ccccc4-c4ccccc4-c4ccccc4)cc3)cc1C2(c1ccccc1)c1ccccc1]

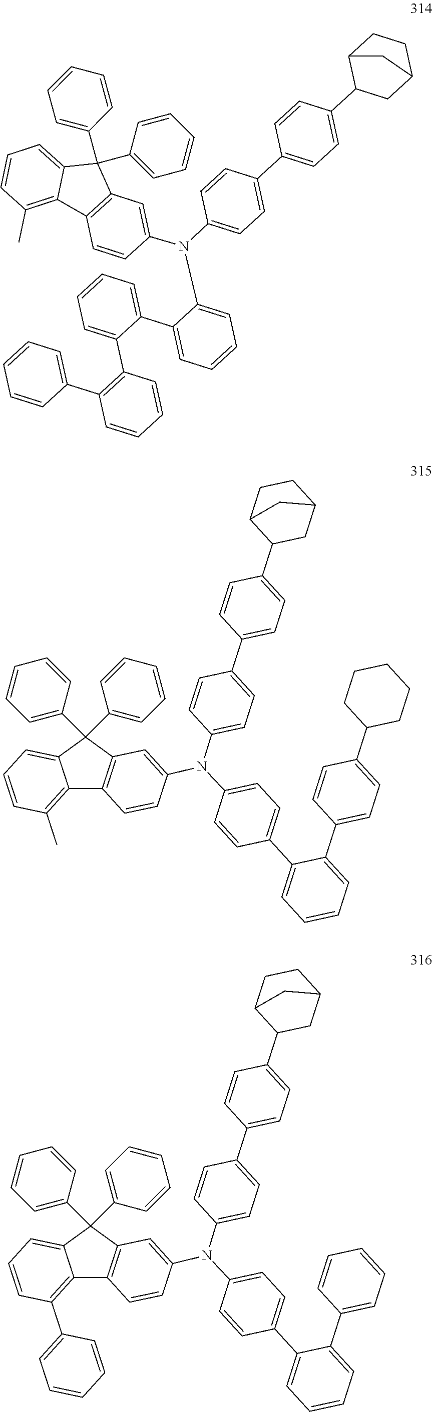 [SMILES: Cc1cccc2c1-c1ccc(N(c3ccc(-c4ccc(C5CC6CCC5C6)cc4)cc3)c3ccccc3-c3ccccc3-c3ccccc3-c3ccccc3)cc1C2(c1ccccc1)c1ccccc1 ; Cc1cccc2c1-c1ccc(N(c3ccc(-c4ccc(C5CC6CCC5C6)cc4)cc3)c3ccc(-c4ccccc4-c4ccc(C5CCCCC5)cc4)cc3)cc1C2(c1ccccc1)c1ccccc1 ; c1ccc(-c2ccccc2-c2ccc(N(c3ccc(-c4ccc(C5CC6CCC5C6)cc4)cc3)c3ccc4c(c3)C(c3ccccc3)(c3ccccc3)c3cccc(-c5ccccc5)c3-4)cc2)cc1]

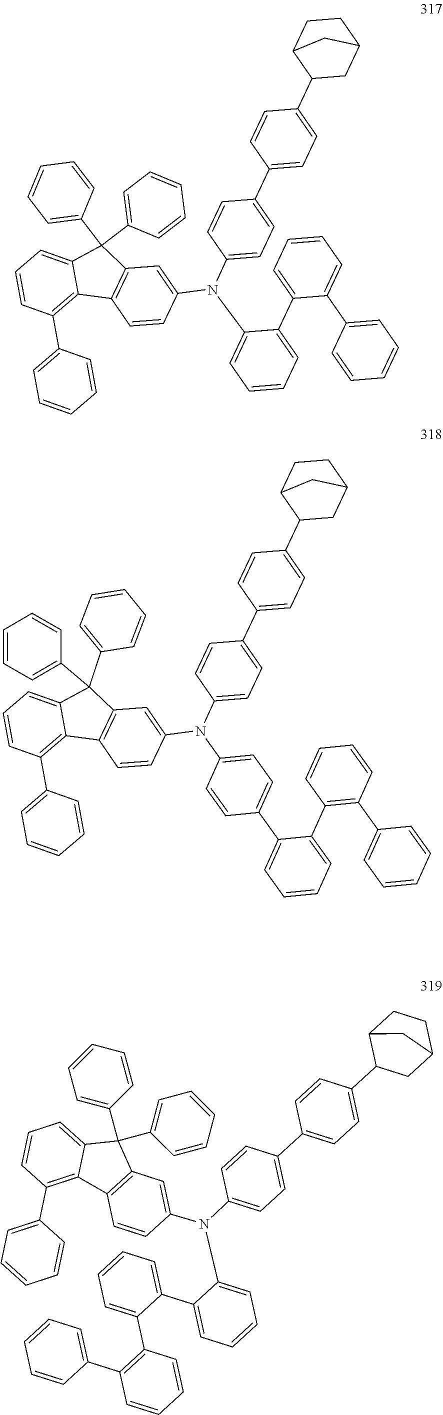 [SMILES: c1ccc(-c2ccccc2-c2ccccc2N(c2ccc(-c3ccc(C4CC5CCC4C5)cc3)cc2)c2ccc3c(c2)C(c2ccccc2)(c2ccccc2)c2cccc(-c4ccccc4)c2-3)cc1 ; c1ccc(-c2ccccc2-c2ccccc2-c2ccc(N(c3ccc(-c4ccc(C5CC6CCC5C6)cc4)cc3)c3ccc4c(c3)C(c3ccccc3)(c3ccccc3)c3cccc(-c5ccccc5)c3-4)cc2)cc1 ; c1ccc(-c2ccccc2-c2ccccc2-c2ccccc2N(c2ccc(-c3ccc(C4CC5CCC4C5)cc3)cc2)c2ccc3c(c2)C(c2ccccc2)(c2ccccc2)c2cccc(-c4ccccc4)c2-3)cc1]

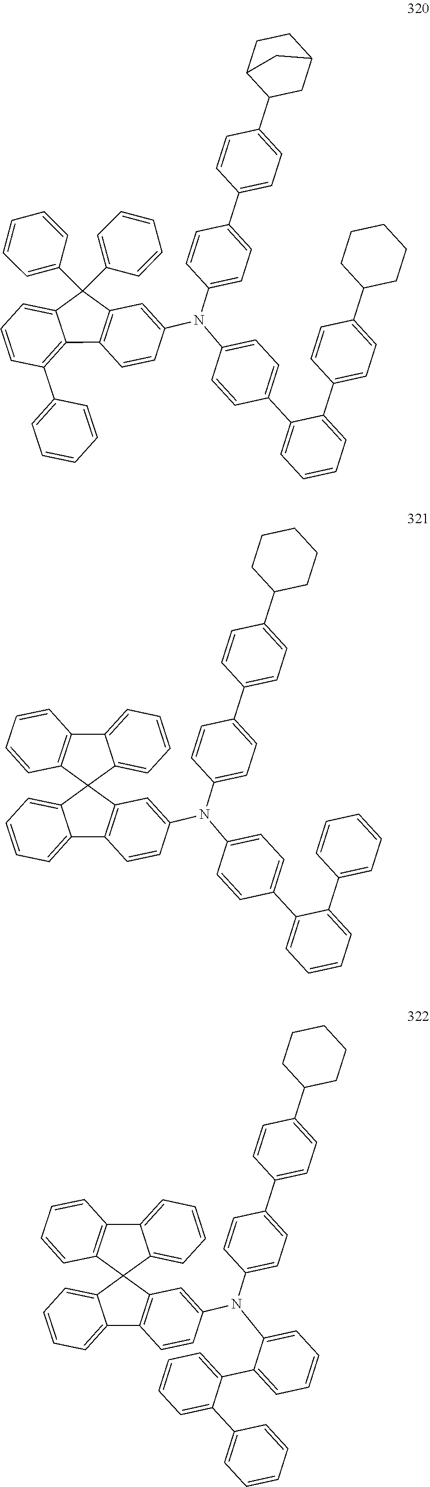 [SMILES: c1ccc(-c2cccc3c2-c2ccc(N(c4ccc(-c5ccc(C6CC7CCC6C7)cc5)cc4)c4ccc(-c5ccccc5-c5ccc(C6CCCCC6)cc5)cc4)cc2C3(c2ccccc2)c2ccccc2)cc1 ; c1ccc(-c2ccccc2-c2ccc(N(c3ccc(-c4ccc(C5CCCCC5)cc4)cc3)c3ccc4c(c3)C3(c5ccccc5-c5ccccc53)c3ccccc3-4)cc2)cc1 ; c1ccc(-c2ccccc2-c2ccccc2N(c2ccc(-c3ccc(C4CCCCC4)cc3)cc2)c2ccc3c(c2)C2(c4ccccc4-c4ccccc42)c2ccccc2-3)cc1]

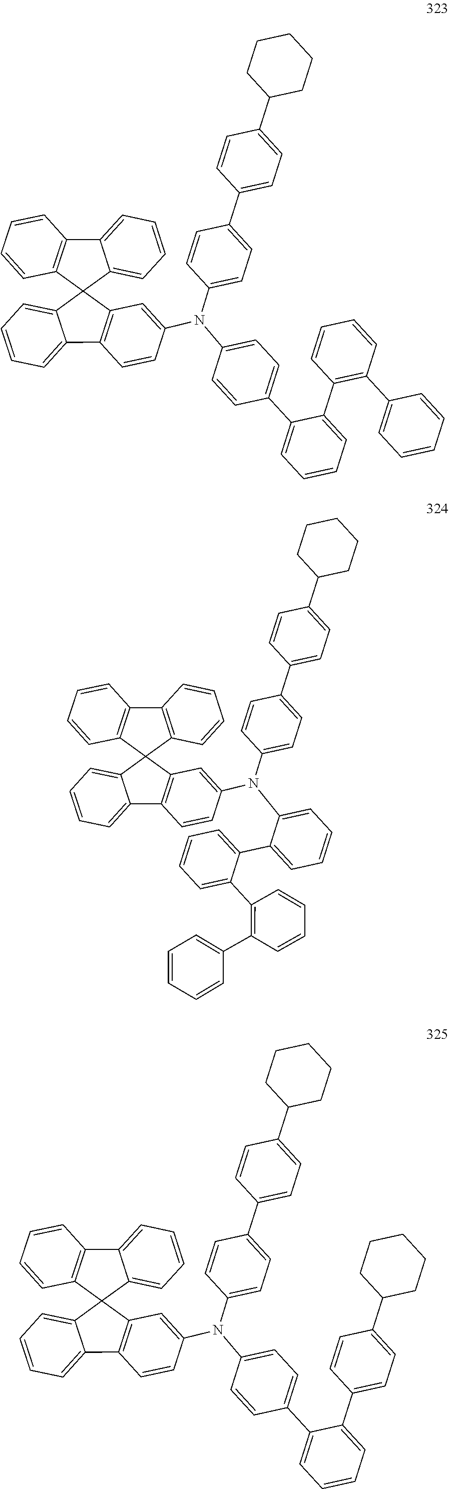

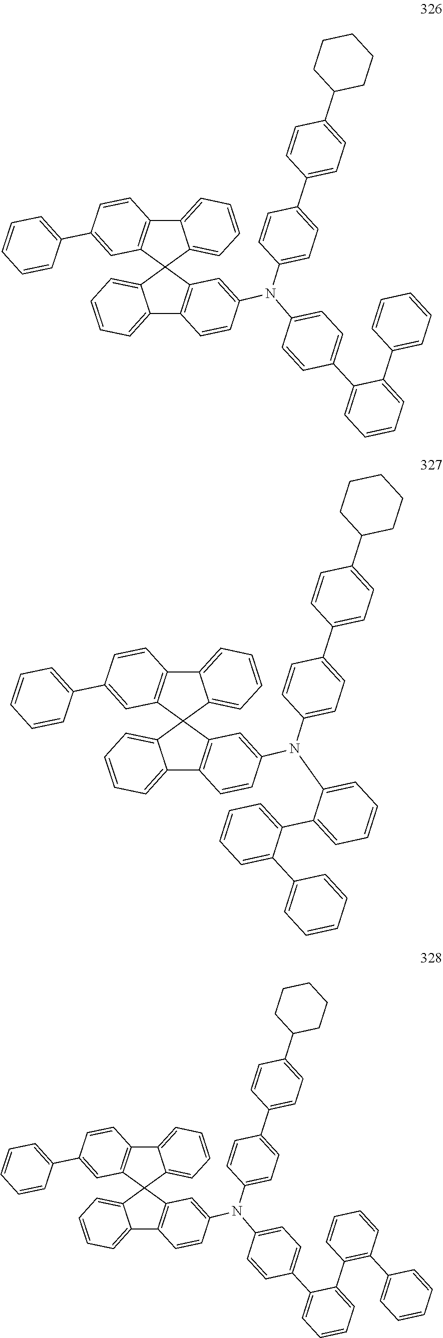

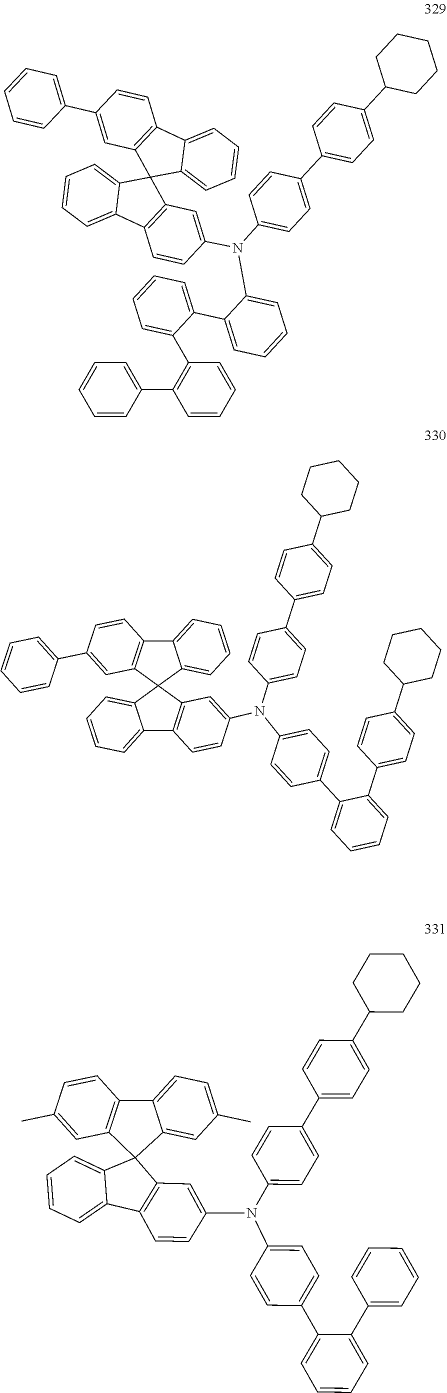 [SMILES: c1ccc(-c2ccc3c(c2)C2(c4ccccc4-3)c3ccccc3-c3ccc(N(c4ccc(-c5ccc(C6CCCCC6)cc5)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccc(C6CCCCC6)cc5)cc4)c4ccc(-c5ccccc5-c5ccc(C6CCCCC6)cc5)cc4)cc32)cc1 ; Cc1ccc2c(c1)C1(c3ccccc3-c3ccc(N(c4ccc(-c5ccc(C6CCCCC6)cc5)cc4)c4ccc(-c5ccccc5-c5ccccc5)cc4)cc31)c1cc(C)ccc1-2]

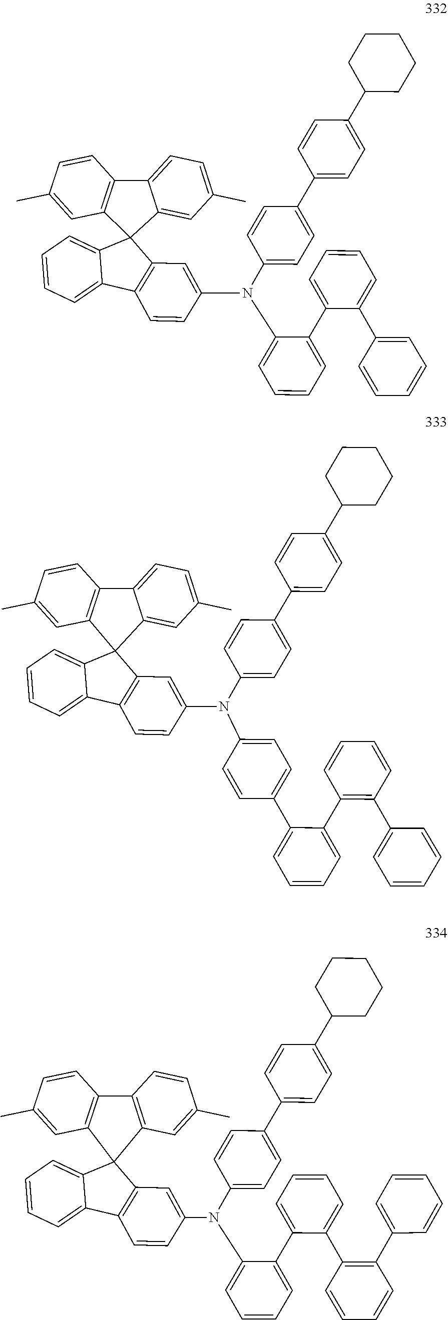 [SMILES: Cc1ccc2c(c1)C1(c3ccccc3-c3ccc(N(c4ccc(-c5ccc(C6CCCCC6)cc5)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4ccc(-c5ccccc5-c5ccccc5-c5ccccc5)cc4)cc31)c1cc(C)ccc1-2 ; Cc1ccc2c(c1)C1(c3ccccc3-c3ccc(N(c4ccc(-c5ccc(C6CCCCC6)cc5)cc4)c4ccccc4-c4ccccc4-c4ccccc4-c4ccccc4)cc31)c1cc(C)ccc1-2]

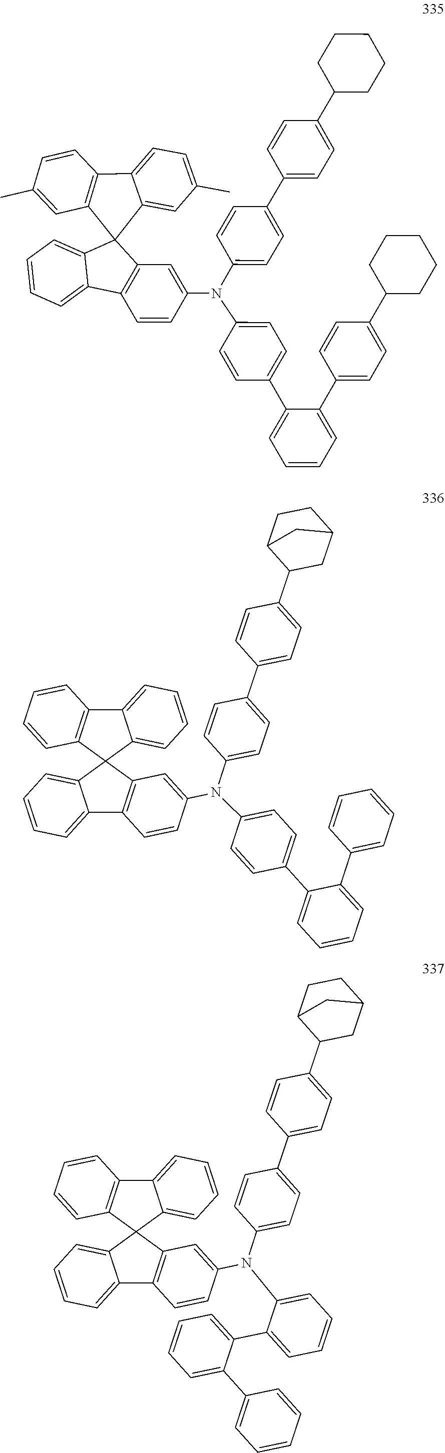

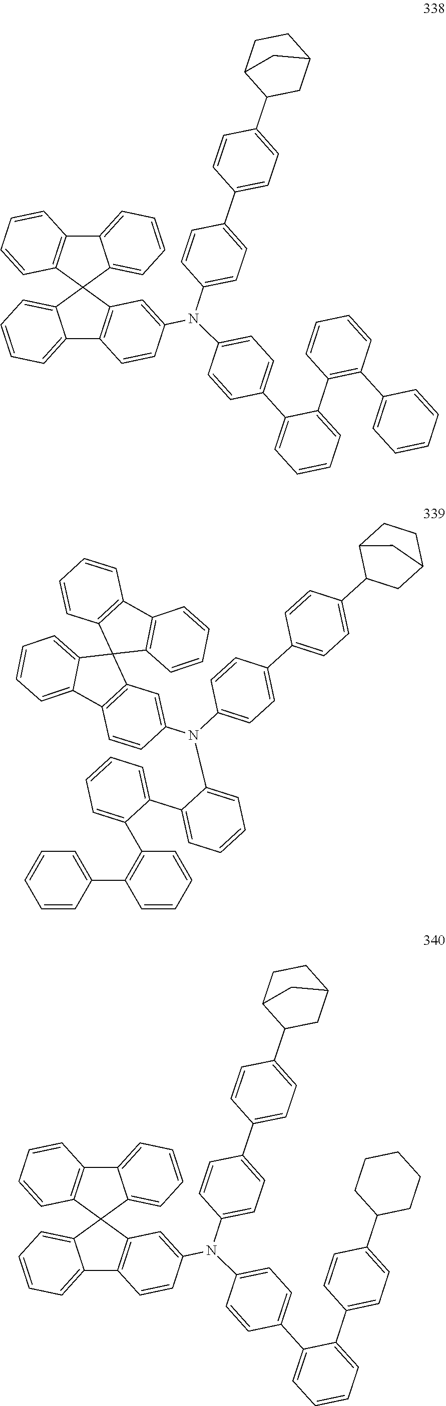 [SMILES: c1ccc(-c2ccccc2-c2ccccc2-c2ccc(N(c3ccc(-c4ccc(C5CC6CCC5C6)cc4)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(-c3ccc(C4CC5CCC4C5)cc3)cc2)c2ccc3c(c2)C2(c4ccccc4-c4ccccc42)c2ccccc2-3)cc1 ; c1ccc(-c2ccc(N(c3ccc(-c4ccc(C5CC6CCC5C6)cc4)cc3)c3ccc4c(c3)C3(c5ccccc5-c5ccccc53)c3ccccc3-4)cc2)c(-c2ccc(C3CCCCC3)cc2)c1]

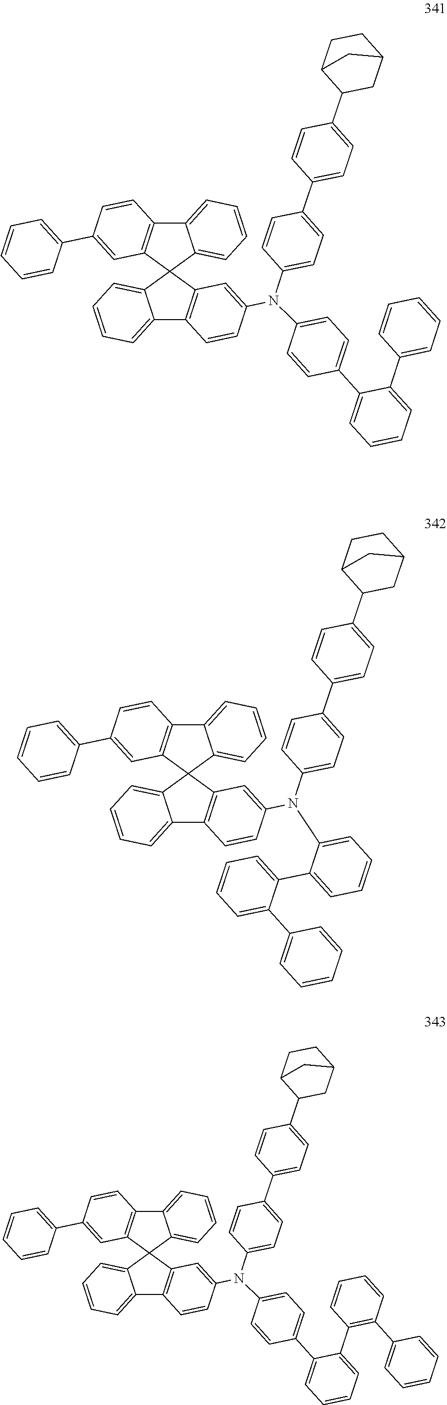 [SMILES: c1ccc(-c2ccc3c(c2)C2(c4ccccc4-3)c3ccccc3-c3ccc(N(c4ccc(-c5ccc(C6CC7CCC6C7)cc5)cc4)c4ccc(-c5ccccc5-c5ccccc5)cc4)cc32)cc1 ; c1ccc(-c2ccc3c(c2)C2(c4ccccc4-3)c3ccccc3-c3ccc(N(c4ccc(-c5ccc(C6CC7CCC6C7)cc5)cc4)c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccc(C6CC7CCC6C7)cc5)cc4)c4ccc(-c5ccccc5-c5ccccc5-c5ccccc5)cc4)cc32)cc1]

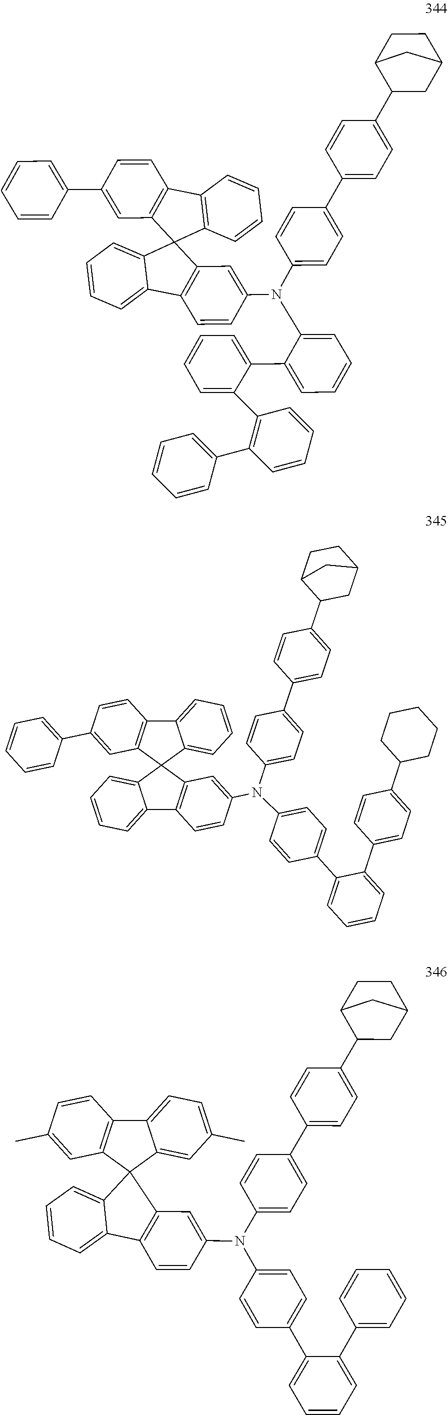 [SMILES: c1ccc(-c2ccc3c(c2)C2(c4ccccc4-3)c3ccccc3-c3ccc(N(c4ccc(-c5ccc(C6CC7CCC6C7)cc5)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccc(C6CC7CCC6C7)cc5)cc4)c4ccc(-c5ccccc5-c5ccc(C6CCCCC6)cc5)cc4)cc32)cc1 ; Cc1ccc2c(c1)C1(c3ccccc3-c3ccc(N(c4ccc(-c5ccc(C6CC7CCC6C7)cc5)cc4)c4ccc(-c5ccccc5-c5ccccc5)cc4)cc31)c1cc(C)ccc1-2]

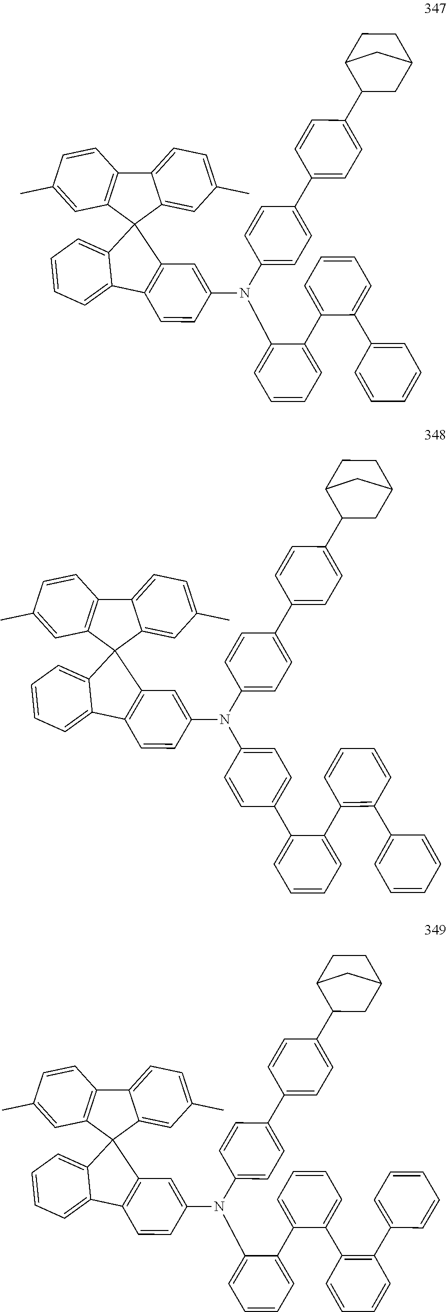 [SMILES: Cc1ccc2c(c1)C1(c3ccccc3-c3ccc(N(c4ccc(-c5ccc(C6CC7CCC6C7)cc5)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccc(C6CC7CCC6C7)cc5)cc4)c4ccc(-c5ccccc5-c5ccccc5-c5ccccc5)cc4)cc31)c1cc(C)ccc1-2 ; Cc1ccc2c(c1)C1(c3ccccc3-c3ccc(N(c4ccc(-c5ccc(C6CC7CCC6C7)cc5)cc4)c4ccccc4-c4ccccc4-c4ccccc4-c4ccccc4)cc31)c1cc(C)ccc1-2]

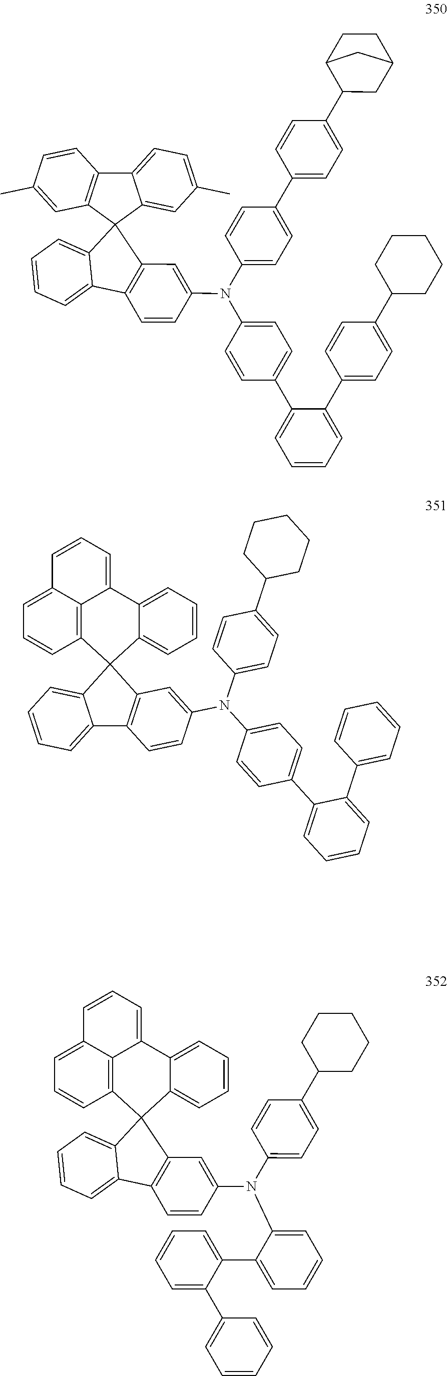 [SMILES: Cc1ccc2c(c1)C1(c3ccccc3-c3ccc(N(c4ccc(-c5ccc(C6CC7CCC6C7)cc5)cc4)c4ccc(-c5ccccc5-c5ccc(C6CCCCC6)cc5)cc4)cc31)c1cc(C)ccc1-2 ; c1ccc(-c2ccccc2-c2ccc(N(c3ccc(C4CCCCC4)cc3)c3ccc4c(c3)C3(c5ccccc5-4)c4ccccc4-c4cccc5cccc3c45)cc2)cc1 ; c1ccc(-c2ccccc2-c2ccccc2N(c2ccc(C3CCCCC3)cc2)c2ccc3c(c2)C2(c4ccccc4-3)c3ccccc3-c3cccc4cccc2c34)cc1]

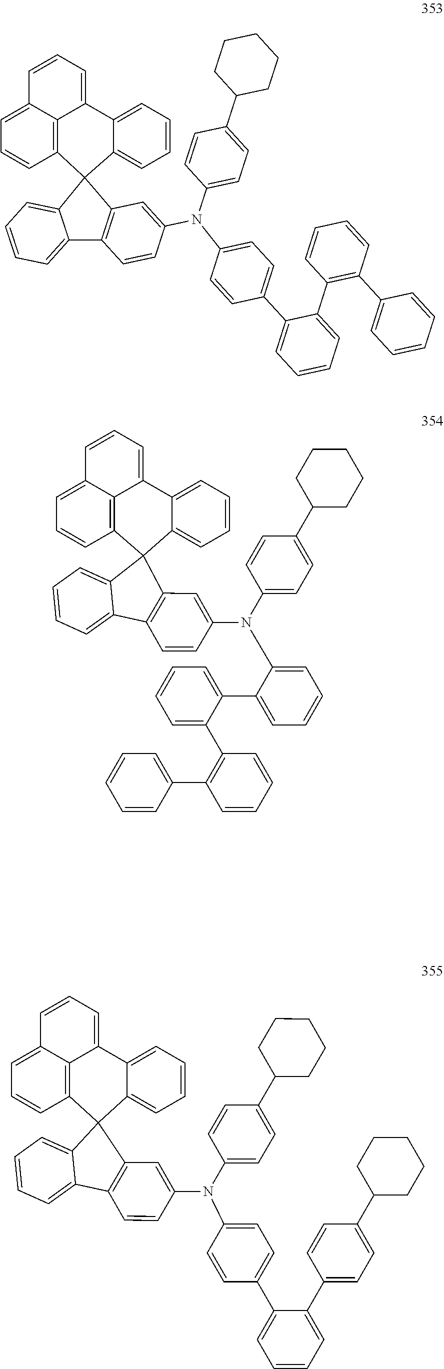 [SMILES: c1ccc(-c2ccccc2-c2ccccc2-c2ccc(N(c3ccc(C4CCCCC4)cc3)c3ccc4c(c3)C3(c5ccccc5-4)c4ccccc4-c4cccc5cccc3c45)cc2)cc1 ; c1ccc(-c2ccccc2-c2ccccc2-c2ccccc2N(c2ccc(C3CCCCC3)cc2)c2ccc3c(c2)C2(c4ccccc4-3)c3ccccc3-c3cccc4cccc2c34)cc1 ; c1ccc(-c2ccc(N(c3ccc(C4CCCCC4)cc3)c3ccc4c(c3)C3(c5ccccc5-4)c4ccccc4-c4cccc5cccc3c45)cc2)c(-c2ccc(C3CCCCC3)cc2)c1]

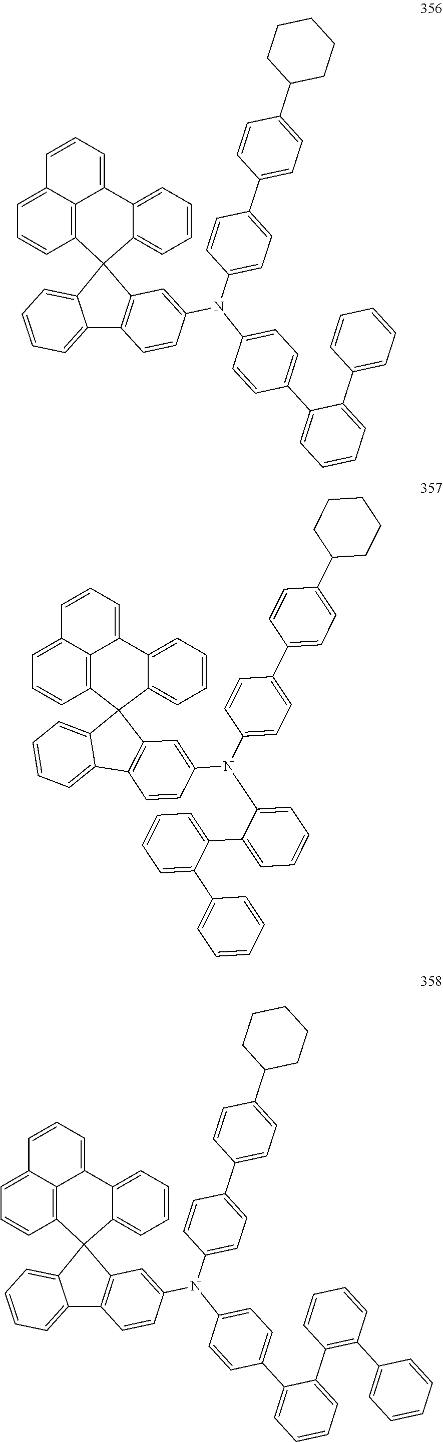

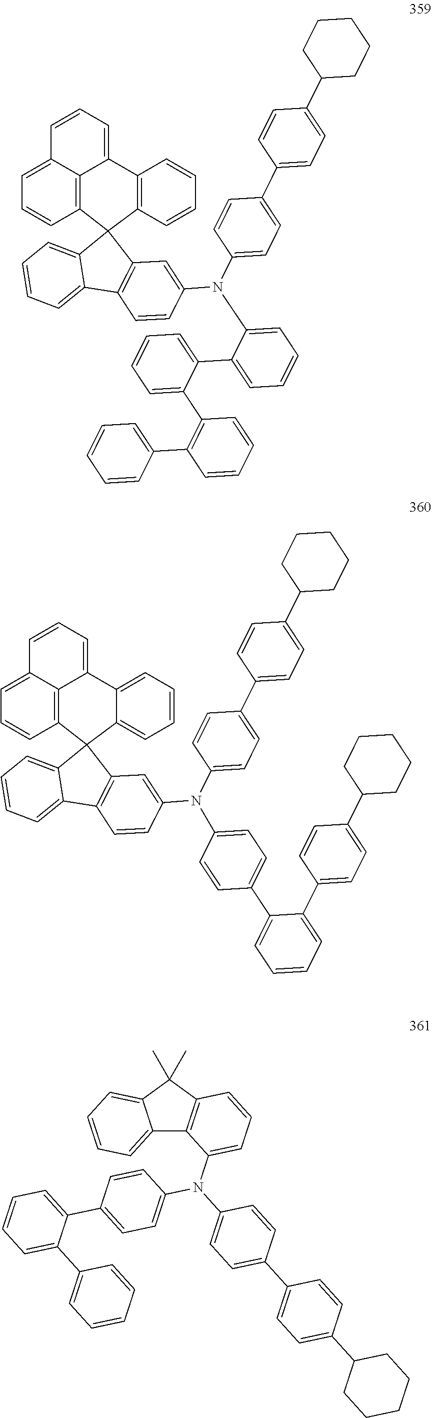 [SMILES: c1ccc(-c2ccccc2-c2ccccc2-c2ccccc2N(c2ccc(-c3ccc(C4CCCCC4)cc3)cc2)c2ccc3c(c2)C2(c4ccccc4-3)c3ccccc3-c3cccc4cccc2c34)cc1 ; c1ccc(-c2ccc(N(c3ccc(-c4ccc(C5CCCCC5)cc4)cc3)c3ccc4c(c3)C3(c5ccccc5-4)c4ccccc4-c4cccc5cccc3c45)cc2)c(-c2ccc(C3CCCCC3)cc2)c1 ; CC1(C)c2ccccc2-c2c(N(c3ccc(-c4ccc(C5CCCCC5)cc4)cc3)c3ccc(-c4ccccc4-c4ccccc4)cc3)cccc21]

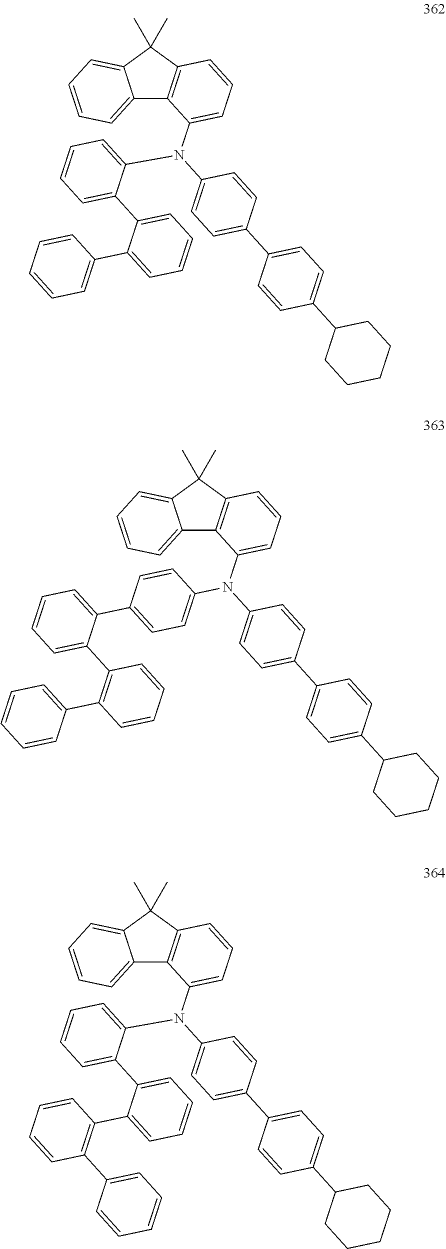 [SMILES: CC1(C)c2ccccc2-c2c(N(c3ccc(-c4ccc(C5CCCCC5)cc4)cc3)c3ccccc3-c3ccccc3-c3ccccc3)cccc21 ; CC1(C)c2ccccc2-c2c(N(c3ccc(-c4ccc(C5CCCCC5)cc4)cc3)c3ccc(-c4ccccc4-c4ccccc4-c4ccccc4)cc3)cccc21 ; CC1(C)c2ccccc2-c2c(N(c3ccc(-c4ccc(C5CCCCC5)cc4)cc3)c3ccccc3-c3ccccc3-c3ccccc3-c3ccccc3)cccc21]

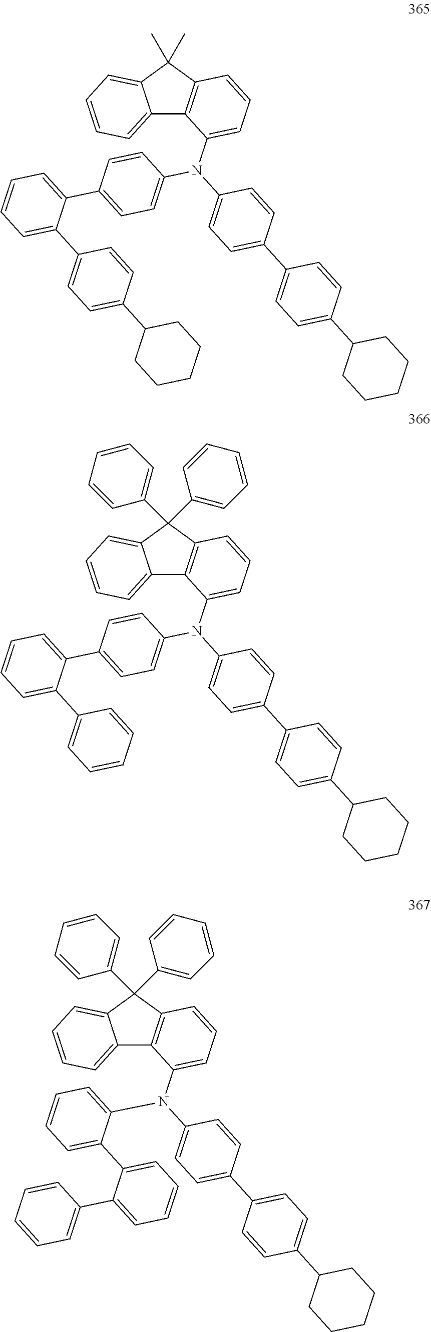 [SMILES: CC1(C)c2ccccc2-c2c(N(c3ccc(-c4ccc(C5CCCCC5)cc4)cc3)c3ccc(-c4ccccc4-c4ccc(C5CCCCC5)cc4)cc3)cccc21 ; c1ccc(-c2ccccc2-c2ccc(N(c3ccc(-c4ccc(C5CCCCC5)cc4)cc3)c3cccc4c3-c3ccccc3C4(c3ccccc3)c3ccccc3)cc2)cc1 ; c1ccc(-c2ccccc2-c2ccccc2N(c2ccc(-c3ccc(C4CCCCC4)cc3)cc2)c2cccc3c2-c2ccccc2C3(c2ccccc2)c2ccccc2)cc1]

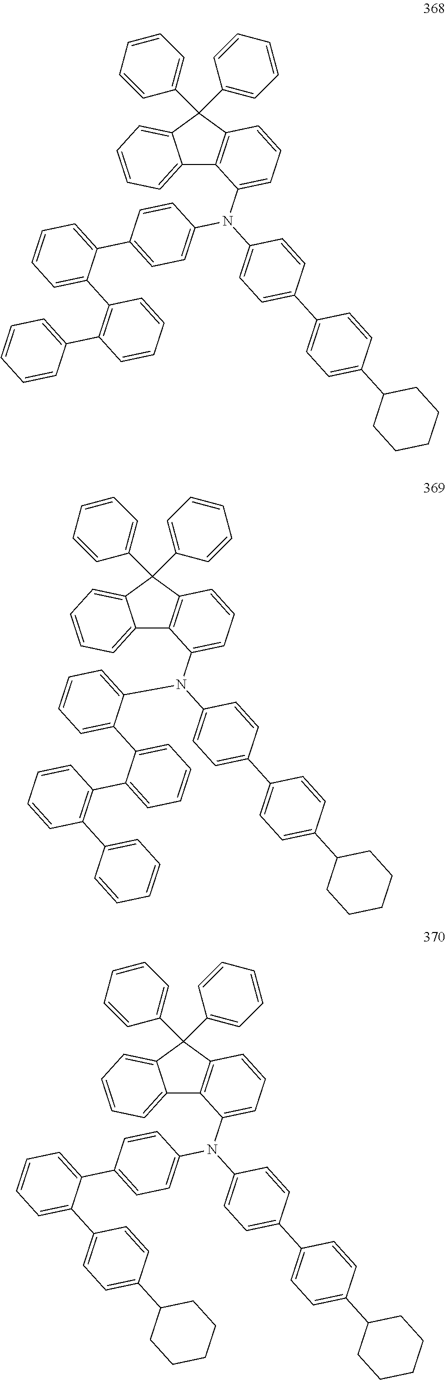 [SMILES: c1ccc(-c2ccccc2-c2ccccc2-c2ccc(N(c3ccc(-c4ccc(C5CCCCC5)cc4)cc3)c3cccc4c3-c3ccccc3C4(c3ccccc3)c3ccccc3)cc2)cc1 ; c1ccc(-c2ccccc2-c2ccccc2-c2ccccc2N(c2ccc(-c3ccc(C4CCCCC4)cc3)cc2)c2cccc3c2-c2ccccc2C3(c2ccccc2)c2ccccc2)cc1 ; c1ccc(C2(c3ccccc3)c3ccccc3-c3c(N(c4ccc(-c5ccc(C6CCCCC6)cc5)cc4)c4ccc(-c5ccccc5-c5ccc(C6CCCCC6)cc5)cc4)cccc32)cc1]

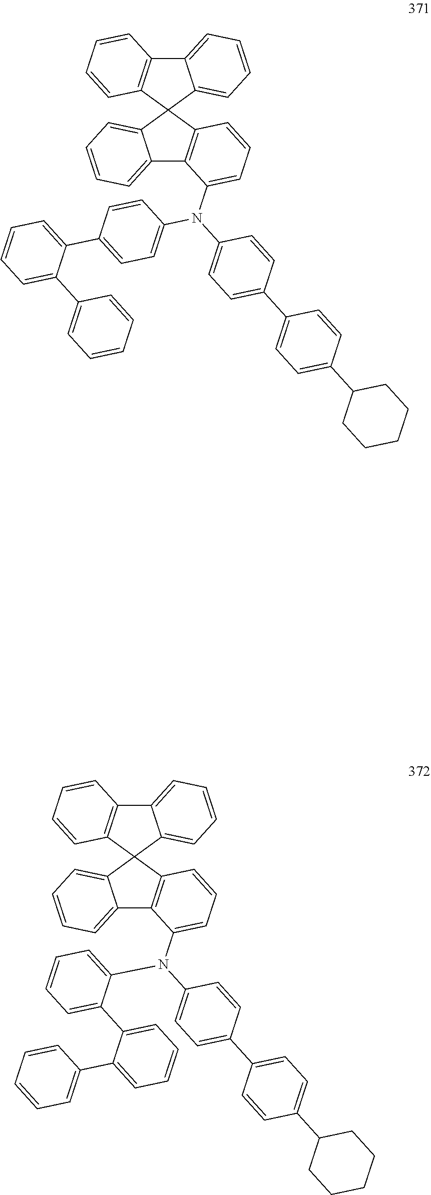 [SMILES: c1ccc(-c2ccccc2-c2ccc(N(c3ccc(-c4ccc(C5CCCCC5)cc4)cc3)c3cccc4c3-c3ccccc3C43c4ccccc4-c4ccccc43)cc2)cc1 ; c1ccc(-c2ccccc2-c2ccccc2N(c2ccc(-c3ccc(C4CCCCC4)cc3)cc2)c2cccc3c2-c2ccccc2C32c3ccccc3-c3ccccc32)cc1]

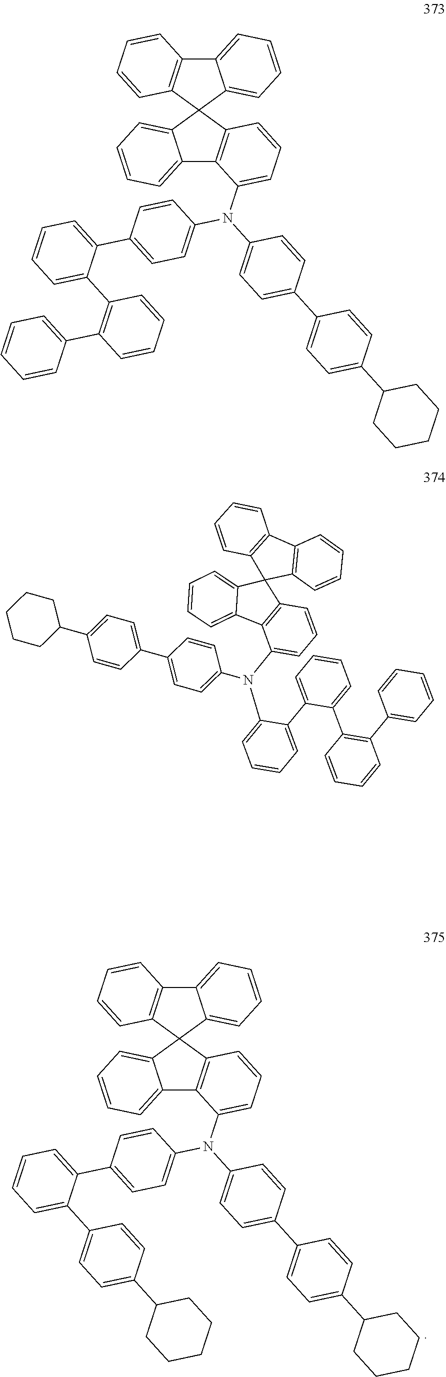 [SMILES: c1ccc(-c2ccccc2-c2ccccc2-c2ccc(N(c3ccc(-c4ccc(C5CCCCC5)cc4)cc3)c3cccc4c3-c3ccccc3C43c4ccccc4-c4ccccc43)cc2)cc1 ; c1ccc(-c2ccccc2-c2ccccc2-c2ccccc2N(c2ccc(-c3ccc(C4CCCCC4)cc3)cc2)c2cccc3c2-c2ccccc2C32c3ccccc3-c3ccccc32)cc1 ; c1ccc(-c2ccc(N(c3ccc(-c4ccc(C5CCCCC5)cc4)cc3)c3cccc4c3-c3ccccc3C43c4ccccc4-c4ccccc43)cc2)c(-c2ccc(C3CCCCC3)cc2)c1]

The amine-containing compound represented by Formula 1 may reduce intermolecular interaction by including L₃, thereby having low packing density and low refractive properties, and a refractive index of the molecule may be changed by varying the substituents of L₃.

In some embodiments, by including R1 in the amine-containing compound, π-conjugation of the molecule may be widely extended, and stability of the molecule in a Polaron state may be increased, so that device lifespan and efficiency may be improved, and a glass transition temperature may be greatly increased by increasing the molecular weight. In some embodiments, by changing the type or kind and position of the substituent in R1, the energy level may be varied and tuned, and through such a variation, the hole injection barrier of the ITO and hole transfer layer may be varied. In some embodiments, the exciton generation efficiency inside the emission layer may be increased by controlling the appropriate or suitable energy level between the hole transfer layer and the emission layer.

As a result, an organic electroluminescent device employing the amine-containing compound represented by Formula 1 may have characteristics of high efficiency, low voltage, high luminance, and long lifespan.

Methods of synthesizing the amine-containing compound represented by Formula 1 may be easily understood to those of ordinary skill in the art by referring to Synthesis Examples and/or Examples described herein.

In one or more embodiments, at least one of the amine-containing compounds represented by Formula 1 may be utilized in a light-emitting device (e.g., an organic light-emitting device). Accordingly, a light-emitting device may include: a first electrode; a second electrode facing the first electrode; an interlayer between the first electrode and the second electrode and including an emission layer; and an amine-containing compound represented by Formula 1 as described herein.

In one or more embodiments,
the first electrode of the light-emitting device may be an anode,
the second electrode of the light-emitting device may be a cathode,
the interlayer may further include a hole transport region between the first electrode and the emission layer and an electron transport region between the emission layer and the second electrode,
the hole transport region may include a hole injection layer, a hole transport layer, an emission auxiliary layer, an electron blocking layer, or any combination thereof, and

The electron transport region may include a hole blocking layer, an electron transport layer, an electron injection layer, an electron control layer, or a combination thereof.

In one or more embodiments, the amine-containing compound represented by Formula 1 may be included in the interlayer.

In one or more embodiments, the amine-containing compound represented by Formula 1 may be included in the hole transport region.

In one or more embodiments, the amine-containing compound represented by Formula 1 may be included in the hole transport layer.

In one or more embodiments, the amine-containing compound represented by Formula 1 may be included in the hole transport layer, and the hole transport layer and the emission layer may be in direct contact with each other.

In one or more embodiments, the emission layer may be to emit blue light. For example, the emission layer may be to emit blue light having a maximum emission wavelength in a range of about 400 nanometers (nm) to about 500 nm, about 410 nm to about 490 nm, about 420 nm to about 480 nm, or about 430 nm to about 470 nm.

In one or more embodiments, the emission layer of the light-emitting device may include a dopant and a host.

In one or more embodiments, the electron transport region in the light-emitting device may include a hole blocking layer, and the hole blocking layer may include a phosphine oxide-containing compound, a silicone-containing compound, or any combination thereof. In some embodiments, the hole blocking layer may be in a direct contact with the emission layer.

In one or more embodiments, the light-emitting device may include a capping layer located outside the first electrode or the second electrode. In some embodiments, the light-emitting device may further include a capping layer located outside the first electrode or the second electrode, and the amine-containing compound represented by Formula 1 may be included in the capping layer.

In one or more embodiments, the light-emitting device may further include at least one selected from among a first capping layer located outside the first electrode and a second capping layer located outside the second electrode, and at least one selected from among the first capping layer and the second capping layer may include the amine-containing compound. The first capping layer and the second capping layer may respectively be understood by referring to the descriptions of the first capping layer and the second capping layer provided herein.

In some embodiments, the light-emitting device may include:
a first capping layer located outside the first electrode and including the amine-containing compound represented by Formula 1;
a second capping layer located outside the second electrode and including the amine-containing compound represented by Formula 1; or
the first capping layer and the second capping layer.

The expression that an “(interlayer and/or a capping layer) includes an amine-containing compound” as utilized herein may be construed as meaning that the “(interlayer and/or the capping layer) may include one amine-containing compound of Formula 1 or two or more different amine-containing compounds of Formula 1”.

For example, in some embodiments, the interlayer and/or the capping layer may include Compound 1 only as the amine-containing compound. In those embodiments, Compound 1 may be included in the hole transport layer or the emission layer of the light-emitting device. In some embodiments, the interlayer may include Compounds 1 and 2 as the amine-containing compounds. In those embodiments, Compounds 1 and 2 may be included in substantially the same layer (for example, both (e.g., simultaneously) Compounds 1 and 2 may be included in the emission layer) or in different layers (for example, Compound 1 may be included in the emission layer, and Compound 2 may be included in the hole transport region).

The term “interlayer” as utilized herein refers to a single layer and/or a plurality of all layers located between a first electrode and a second electrode in a light-emitting device.

According to one or more embodiments, an electronic apparatus may include the light-emitting device. The electronic apparatus may further include a thin-film transistor. In some embodiments, the electronic apparatus may further include a thin-film transistor including a source electrode and drain electrode, and the first electrode of the light-emitting device may be electrically connected to the source electrode or the drain electrode. In some embodiments, the electronic apparatus may further include a color filter, a color-conversion layer, a touchscreen layer, a polarizing layer, or any combination thereof. The electronic apparatus may be understood by referring to the description of the electronic apparatus provided herein.

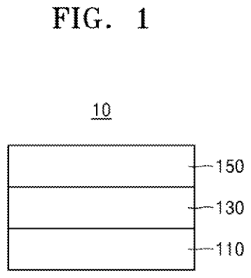

### DESCRIPTION OF FIG. 1

FIG. 1 is a schematic view of a light-emitting device 10 according to one or more embodiments of the present disclosure. The light-emitting device 10 may include a first electrode 110, an interlayer 130, and a second electrode 150.

Hereinafter, the structure of the light-emitting device 10 according to one or more embodiments and a method of manufacturing the light-emitting device 10 according to one or more embodiments will be described in more detail with reference to FIG. 1.

### First Electrode 110

In FIG. 1, in some embodiments, a substrate may be additionally provided and located under the first electrode 110 and/or above (e.g., on) the second electrode 150. The substrate may be a glass substrate or a plastic substrate. The substrate may be a flexible substrate including plastic having excellent or suitable heat resistance and durability, for example, polyimide, polyethylene terephthalate (PET), polycarbonate, polyethylene naphthalate, polyarylate (PAR), polyetherimide, or any combination thereof.

The first electrode 110 may be formed by depositing or sputtering, on the substrate, a material for forming the first electrode 110. When the first electrode 110 is an anode, a high work function material that facilitates hole injection may be utilized as a material for the first electrode.

The first electrode 110 may be a reflective electrode, a semi-transmissive electrode, or a transmissive electrode. In some embodiments, when the first electrode 110 is a transmissive electrode, a material for forming the first electrode 110 may be indium tin oxide (ITO), indium zinc oxide (IZO), tin oxide (SnO₂), zinc oxide (ZnO), or any combination thereof. In some embodiments, when the first electrode 110 is a semi-transmissive electrode or a reflective electrode, magnesium (Mg), silver (Ag), aluminum (Al), aluminum-lithium (Al—Li), calcium (Ca), magnesium-indium (Mg—In), magnesium-silver (Mg—Ag), or any combination thereof may be utilized as a material for forming the first electrode 110.

The first electrode 110 may have a single-layered structure including (e.g., consisting of) a single layer or a multi-layered structure including two or more layers. In some embodiments, the first electrode 110 may have a triple-layered structure of ITO/Ag/ITO.

### Interlayer 130

The interlayer 130 may be on the first electrode 110. The interlayer 130 may include an emission layer.

In one or more embodiments, the interlayer 130 may further include a hole transport region between the first electrode 110 and the emission layer and an electron transport region between the emission layer and the second electrode 150.

In one or more embodiments, the interlayer 130 may further include metal-containing compounds such as organometallic compounds, inorganic materials such as quantum dots, and/or the like, in addition to one or more suitable organic materials.

In one or more embodiments, the interlayer 130 may include: i) at least two emitting units sequentially stacked between the first electrode 110 and the second electrode 150; and ii) a charge generation layer between the at least two emitting units. When the interlayer 130 includes the at least two emitting units and the charge generation layer, the light-emitting device 10 may be a tandem light-emitting device.

### Hole Transport Region in Interlayer 130

The hole transport region may have i) a single-layered structure including (e.g., consisting of) a single layer including (e.g., consisting of) a single material, ii) a single-layered structure including (e.g., consisting of) a single layer including a plurality of different materials, or iii) a multi-layered structure having a plurality of layers including a plurality of different materials.

The hole transport region may include a hole injection layer, a hole transport layer, an emission auxiliary layer, an electron blocking layer, or a combination thereof.

For example, in some embodiments, the hole transport region may have a multi-layered structure, e.g., a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein constituent layers of each structure are sequentially stacked on the first electrode 110 in each stated order.

In some embodiments, the hole transport region may include, as an amine-containing compound, a compound represented by Formula 201, a compound represented by Formula 202, or any combination thereof:

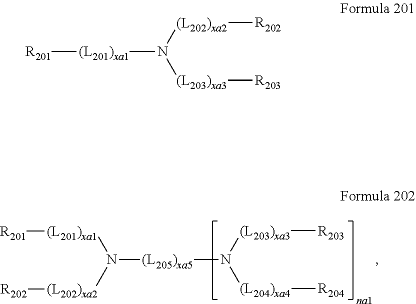

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S—*′, *—N(Q₂₀₁)—*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be bound to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (e.g., a carbazole group and/or the like) unsubstituted or substituted with at least one R₁₀ₐ (e.g., Compound HT16 described herein),
R₂₀₃ and R₂₀₄ may optionally be bound to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and
na1 may be an integer from 1 to 4.

In some embodiments, Formulae 201 and 202 may each include at least one selected from groups represented by Formulae CY201 to CY217:

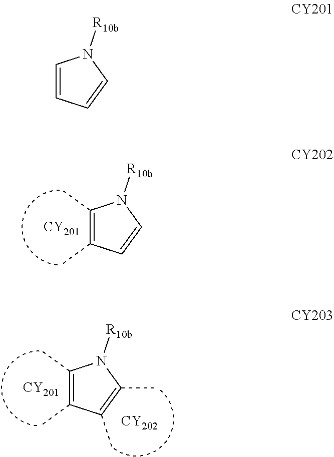

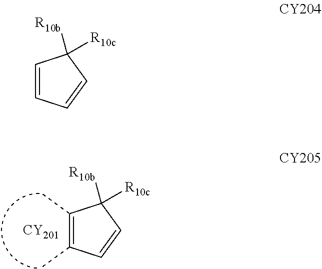

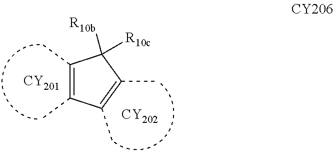

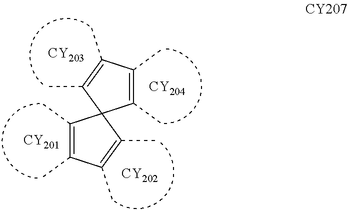

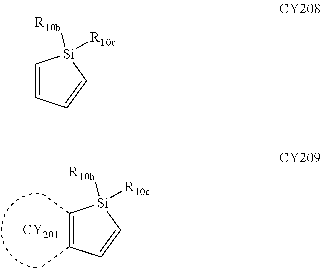

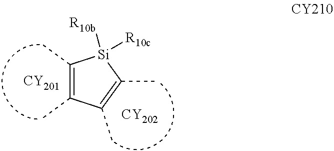

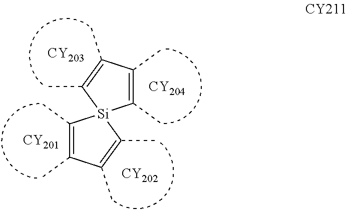

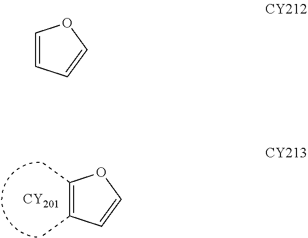

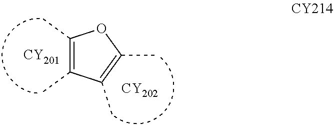

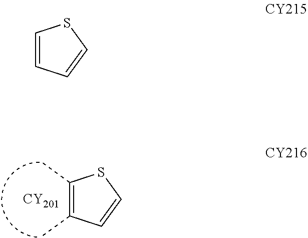

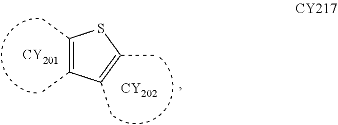

wherein, in Formulae CY201 to CY217, R_{10b} and R_{10c} may each be understood by referring to the descriptions of R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In one or more embodiments, in Formulae CY201 to CY217, ring CY201 to ring CY₂₀₄ may each independently be a benzene group, a naphthalene group, a phenanthrene group, or an anthracene group.

In one or more embodiments, Formulae 201 and 202 may each include at least one selected from the groups represented by Formulae CY201 to CY203.

In one or more embodiments, Formula 201 may include at least one selected from the groups represented by Formulae CY201 to CY203 and at least one selected from the groups represented by Formulae CY204 to CY217.

In one or more embodiments, in Formula 201, xa1 may be 1, R₂₀₁ may be represented by one selected from Formulae CY201 to CY203, xa2 may be 0, and R₂₀₂ may be represented by one selected from Formulae CY204 to CY207.

In one or more embodiments, Formulae 201 and 202 may each not include (e.g., may each exclude) any groups represented by Formulae CY201 to CY203.

In one or more embodiments, Formulae 201 and 202 may each not include (e.g., may each exclude) any groups represented by Formulae CY201 to CY203, and may include at least one selected from the groups represented by Formulae CY204 to CY217.

In one or more embodiments, Formulae 201 and 202 may each not include (e.g., may each exclude) any groups represented by Formulae CY201 to CY217.

In some embodiments, the hole transport region may include one selected from Compounds HT1 to HT46, 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), N,N′-di(naphthalen-1-yl)-N,N′-diphenyl-benzidine (NPB (NPD)), β-NPB, N,N′-bis(3-methylphenyl)-N,N′-diphenyl-[1,1′-biphenyl]-4,4′-diamine (TPD), spiro-TPD, spiro-NPB, methylated-NPB, 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), and polyaniline/poly(4-styrenesulfonate (PANI/PSS), and/or any combination thereof:

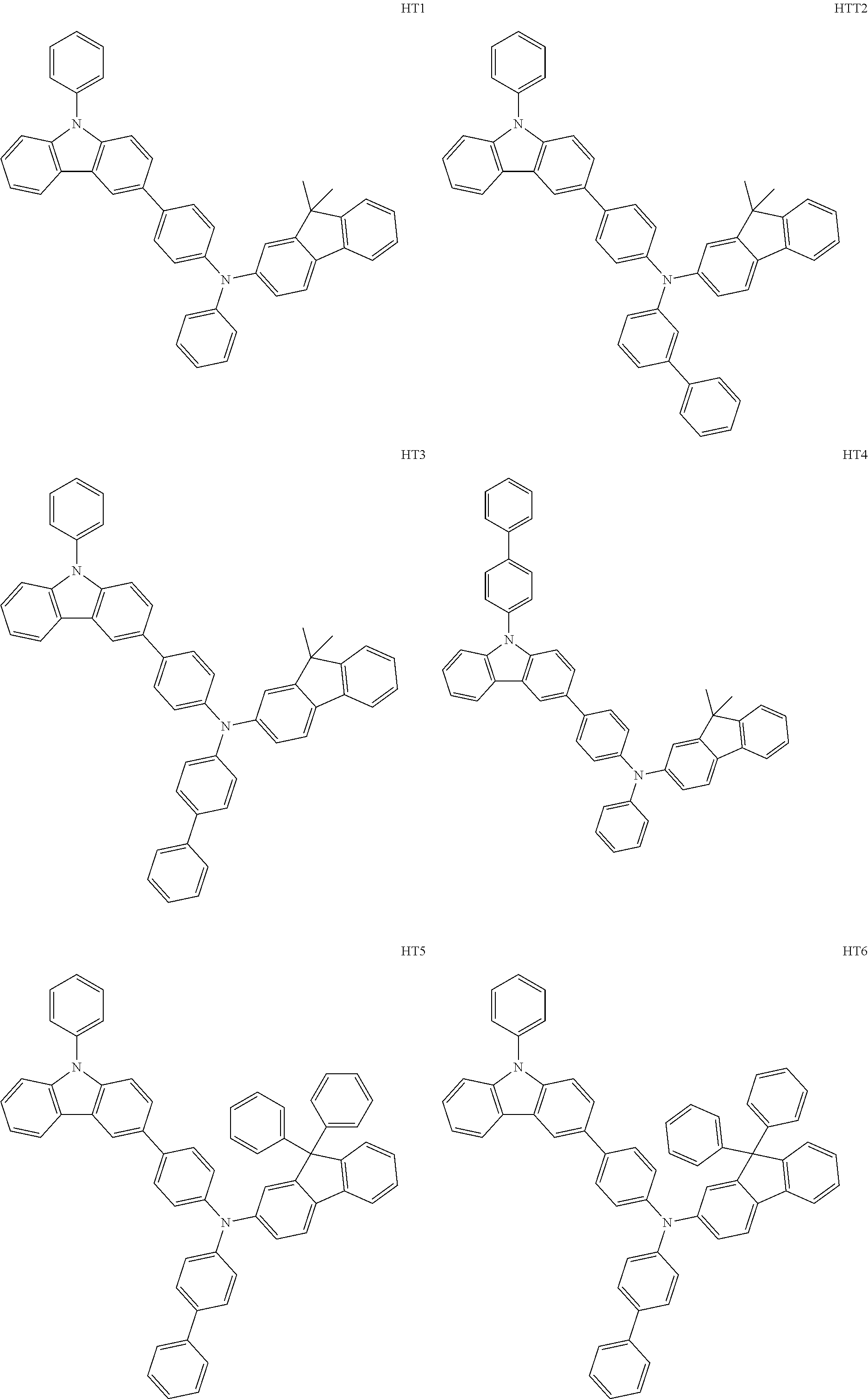

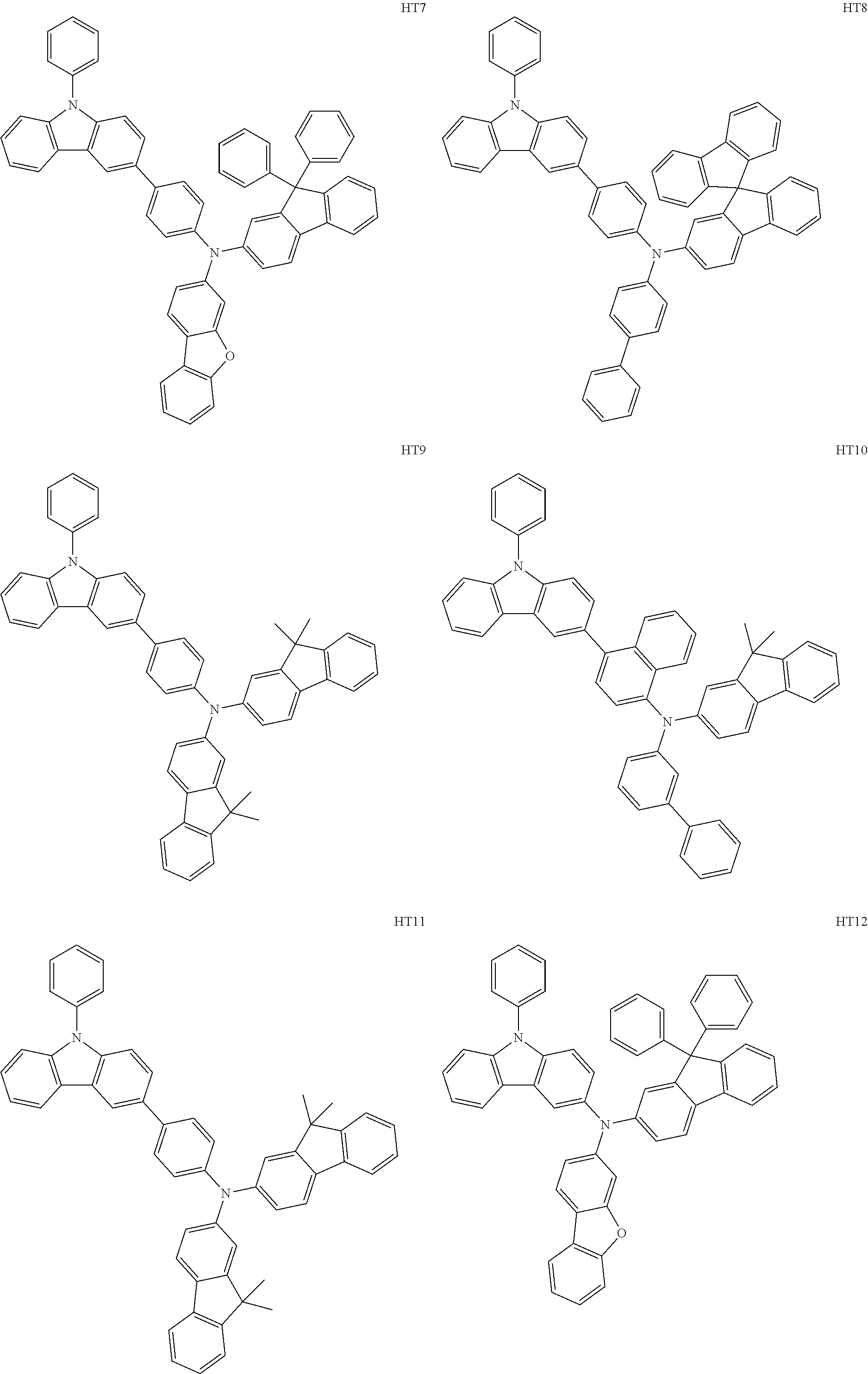

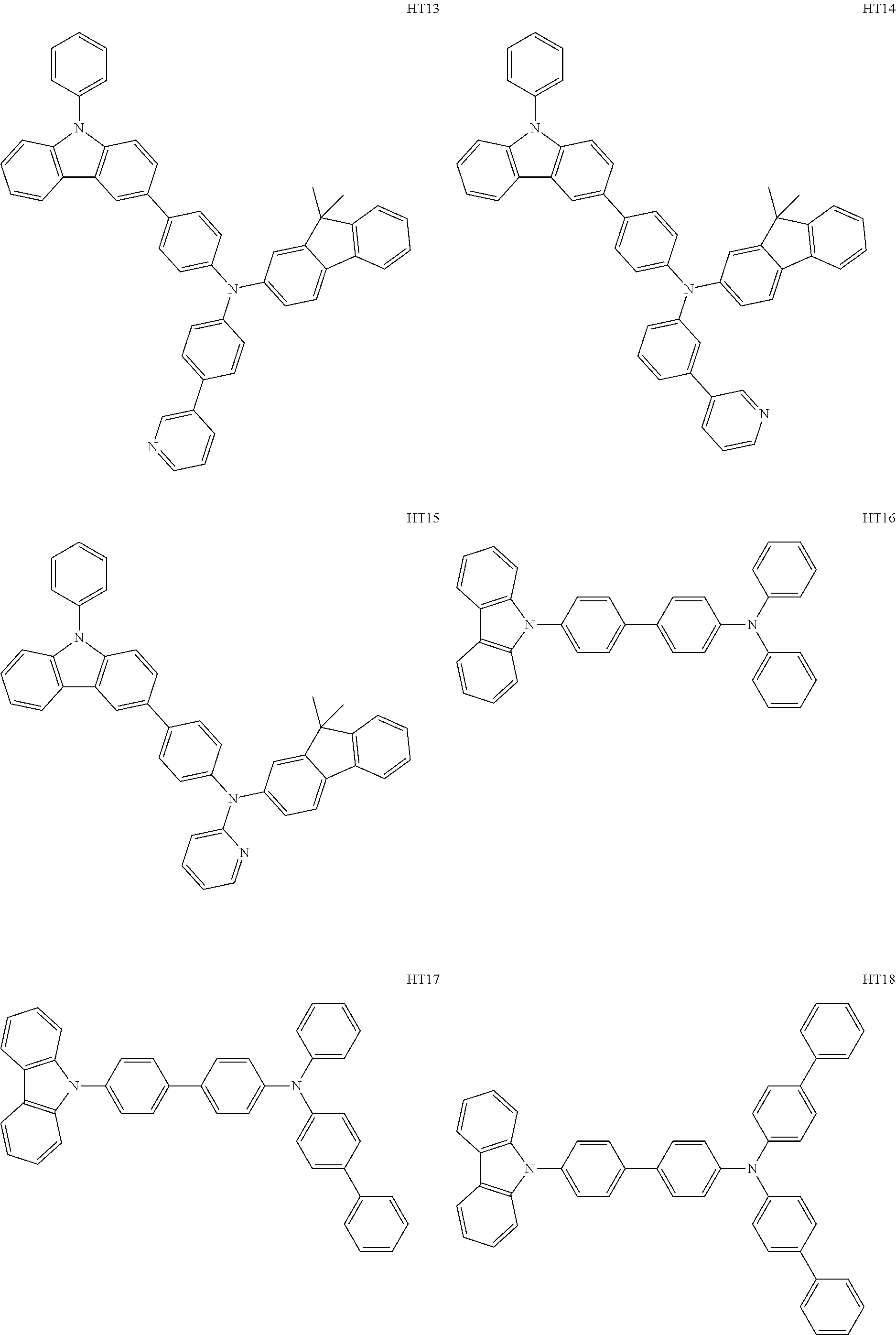 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1 ; c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

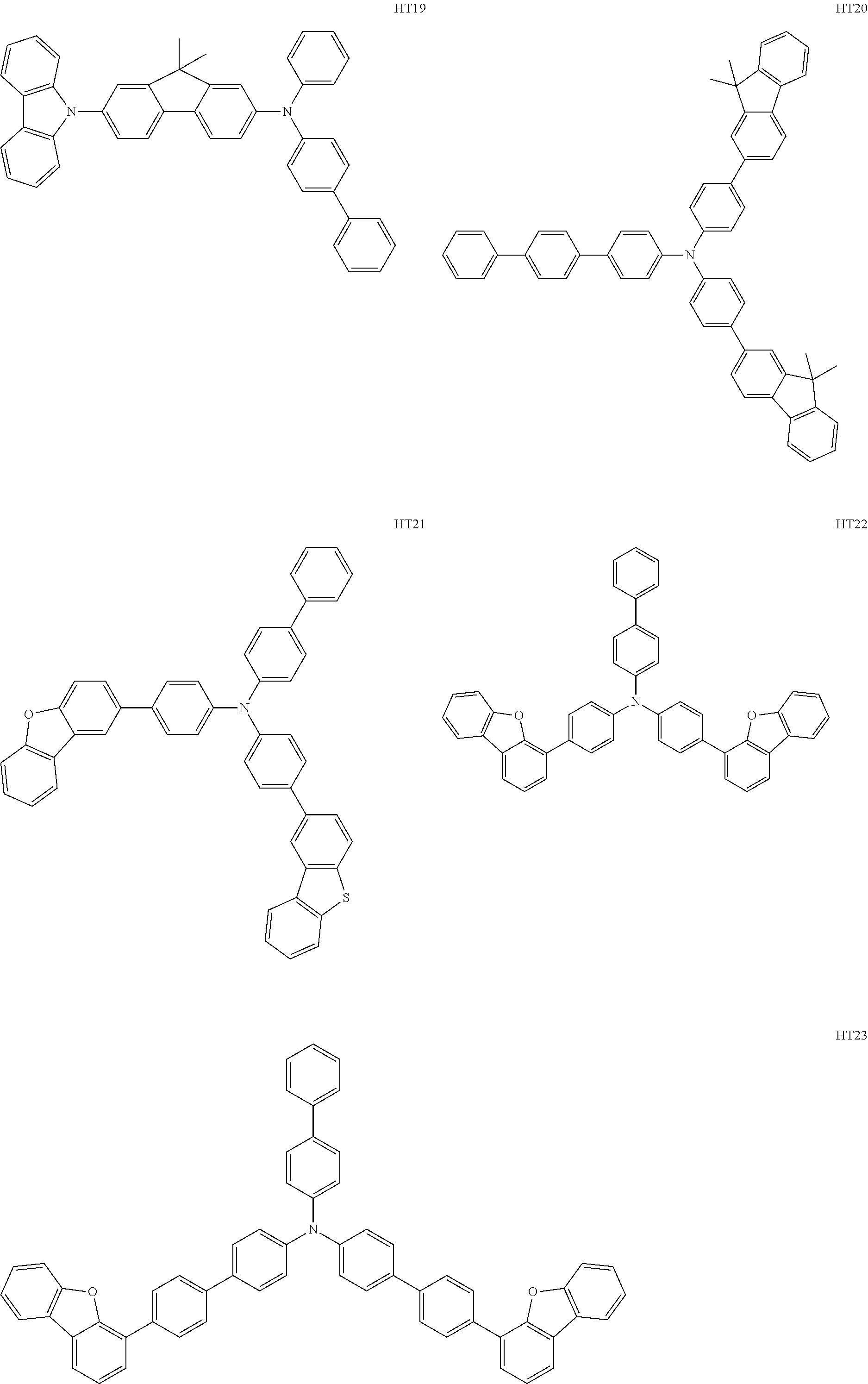 [SMILES: CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21 ; c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

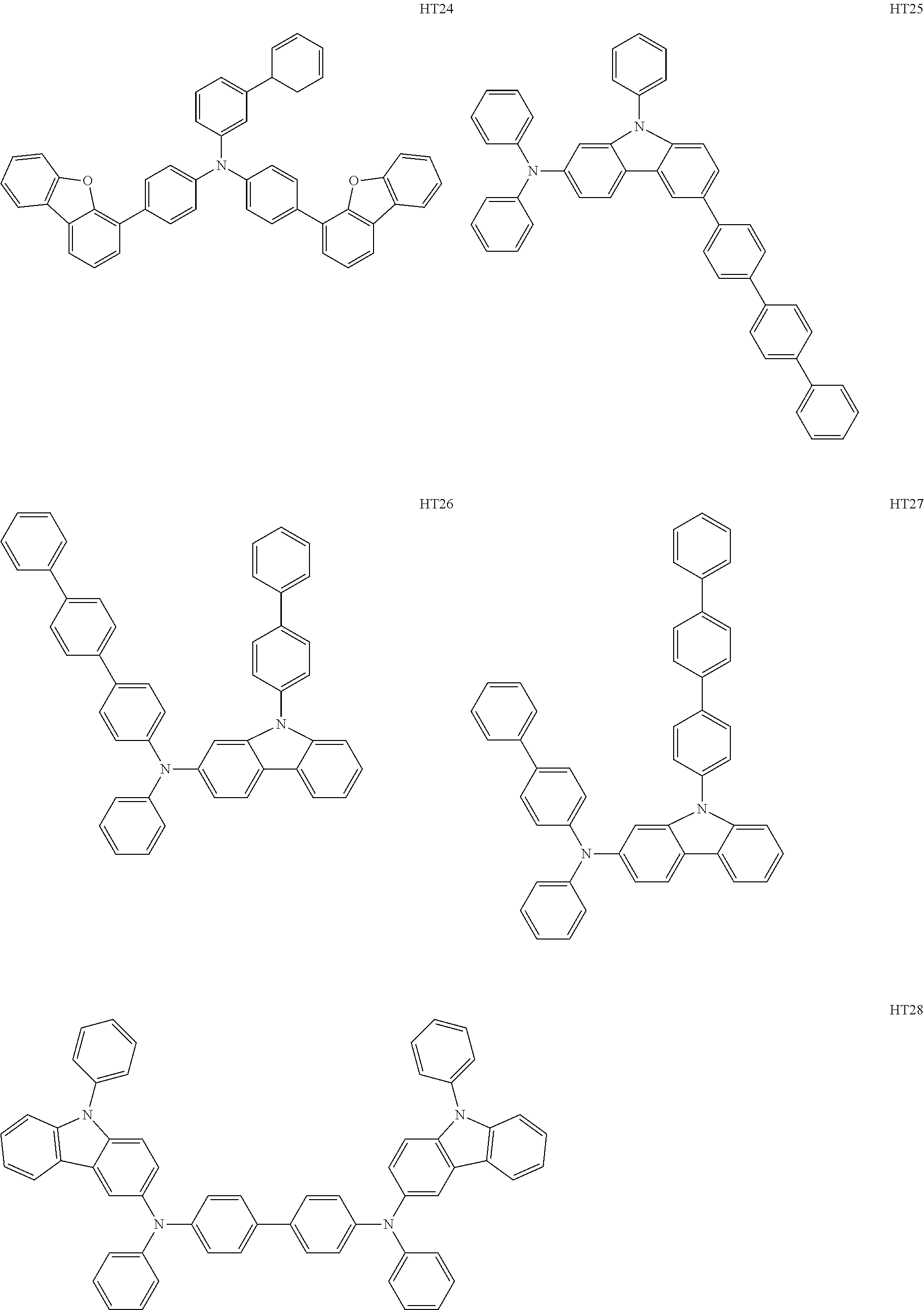 [SMILES: C1=CCC(c2cccc(N(c3ccc(-c4cccc5c4oc4ccccc45)cc3)c3ccc(-c4cccc5c4oc4ccccc45)cc3)c2)C=C1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

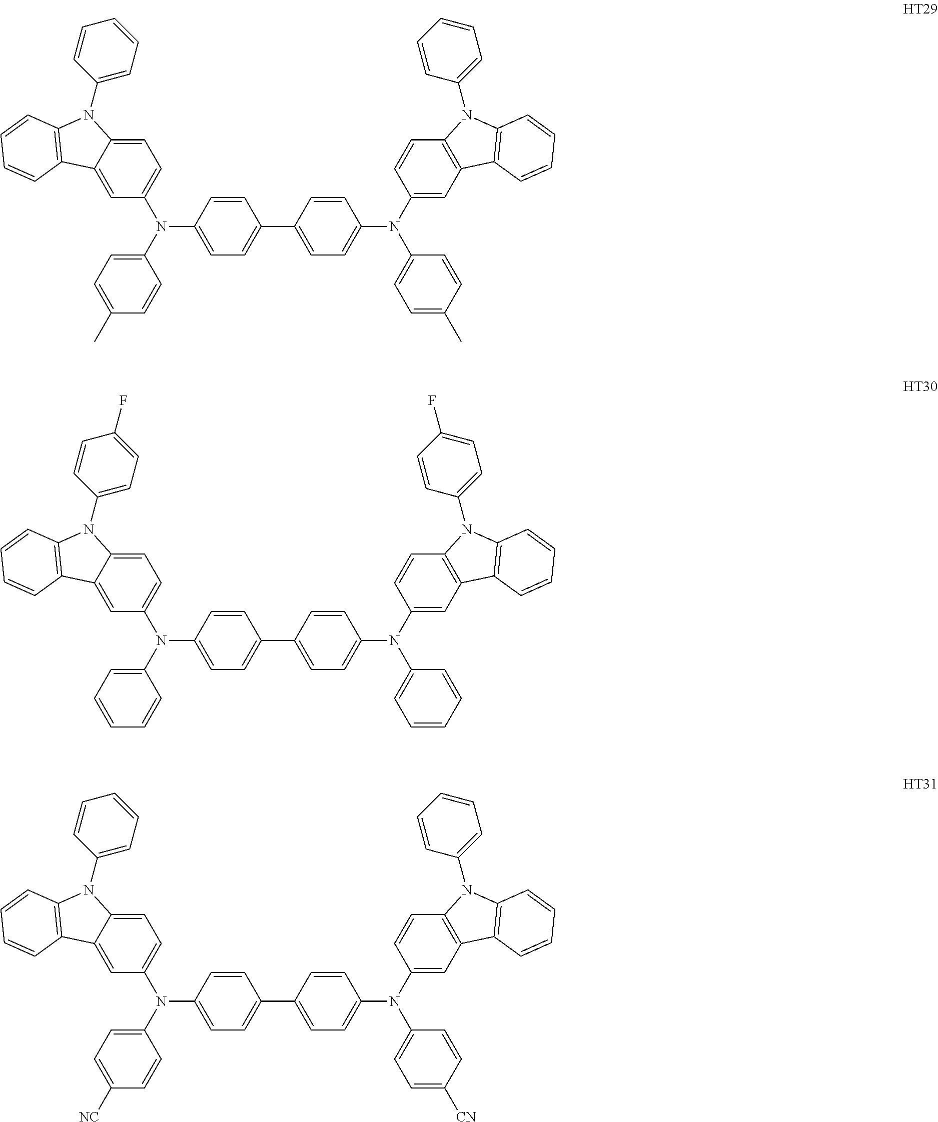 [SMILES: Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

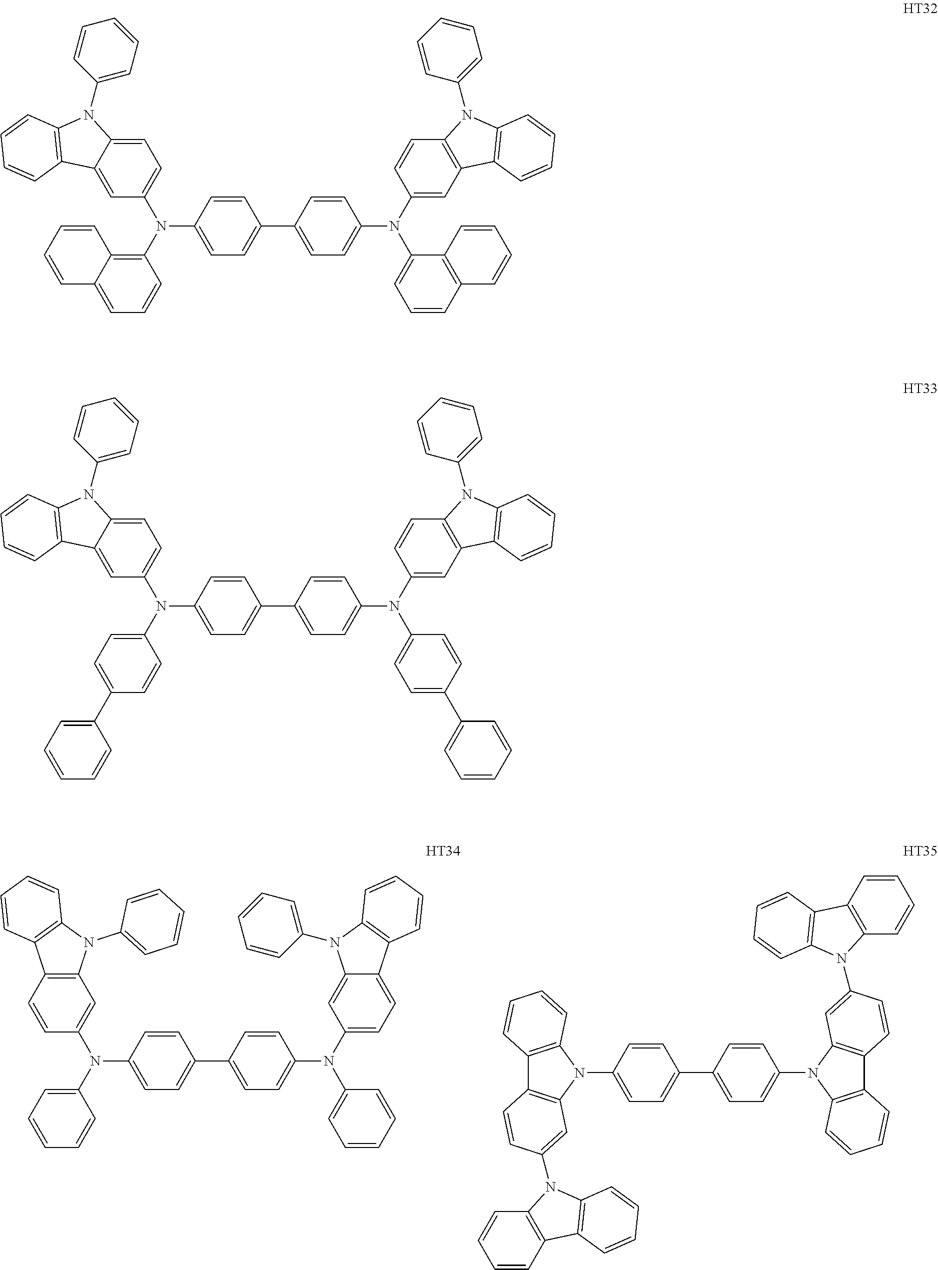 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1]

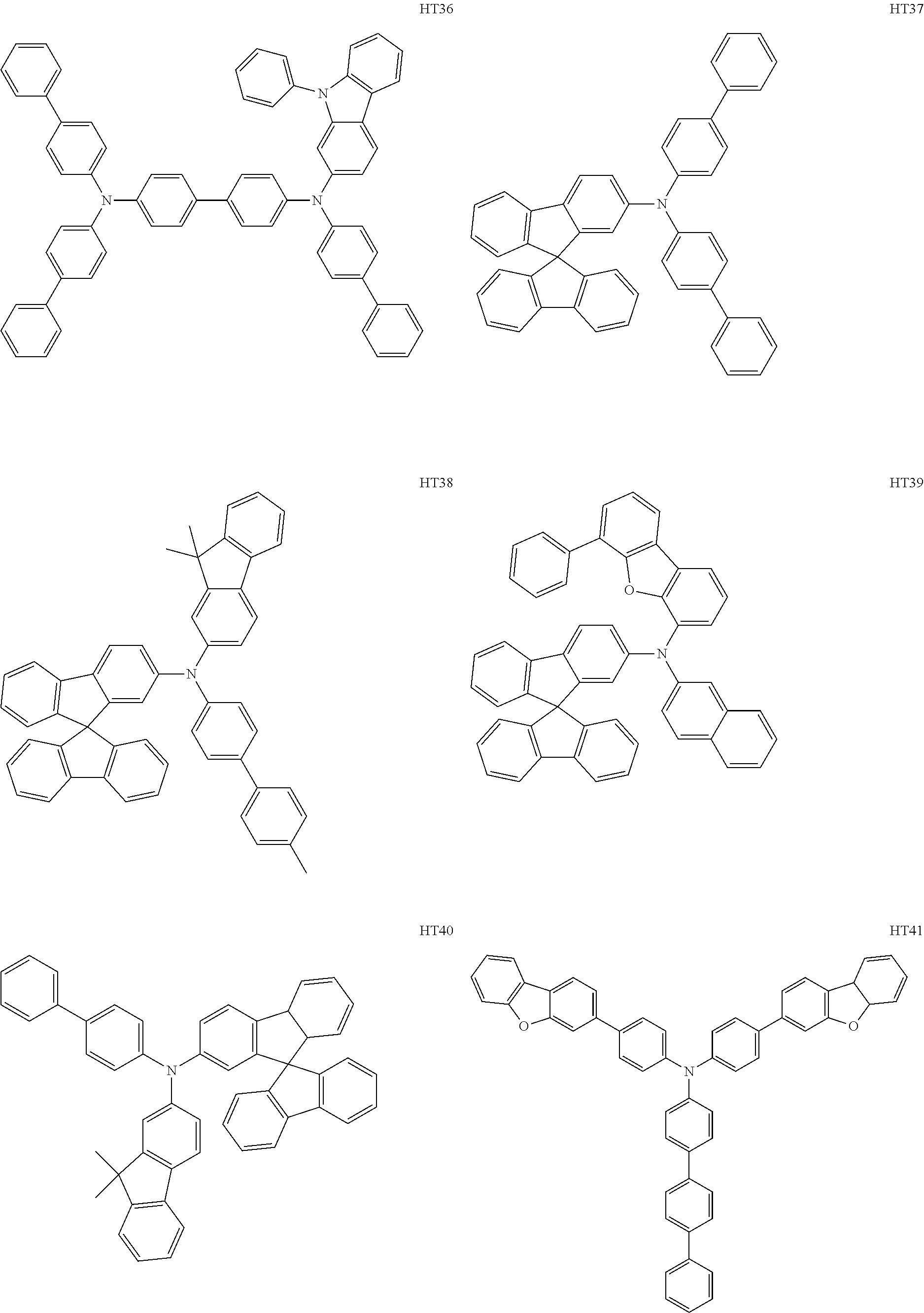

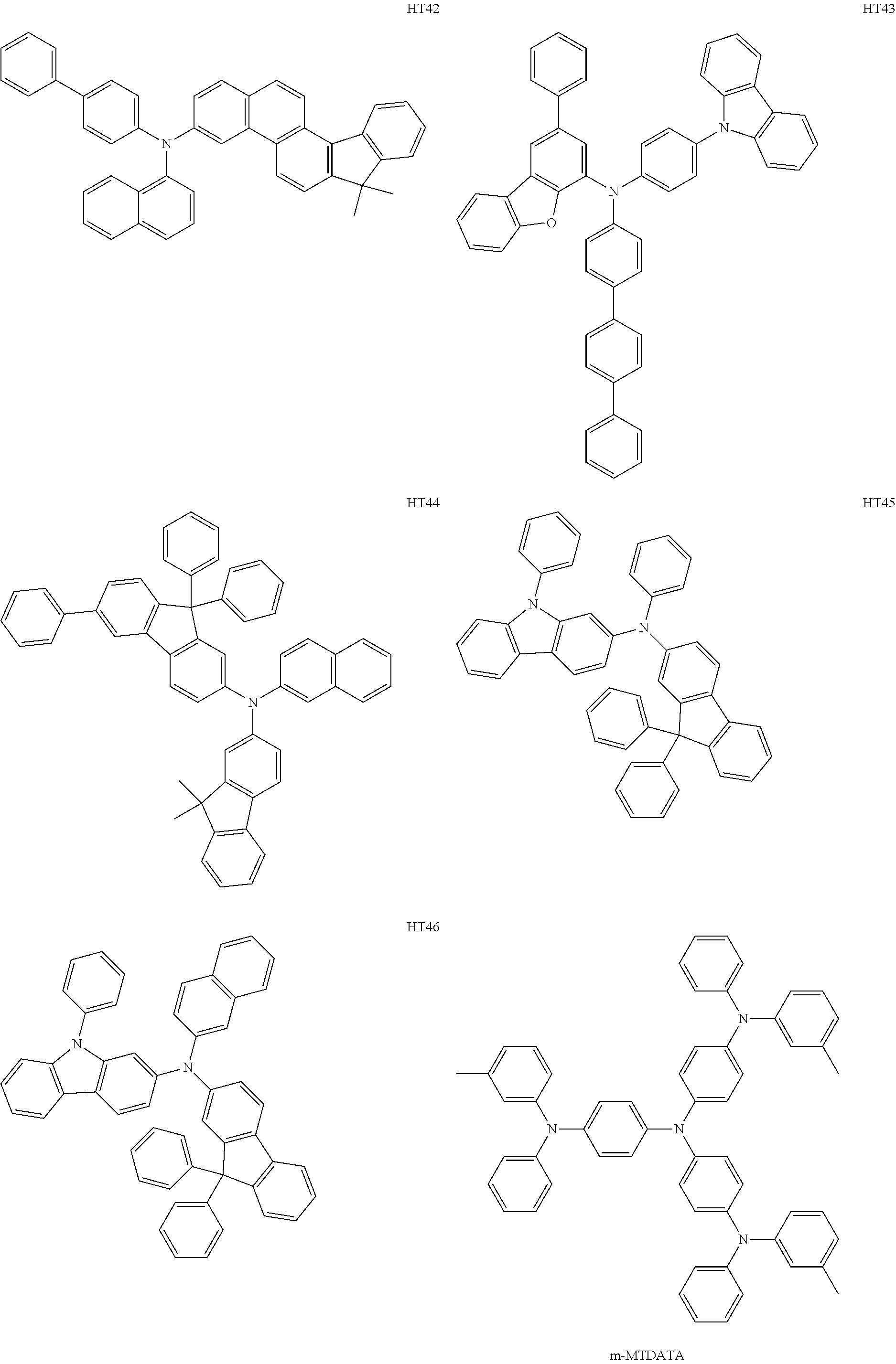

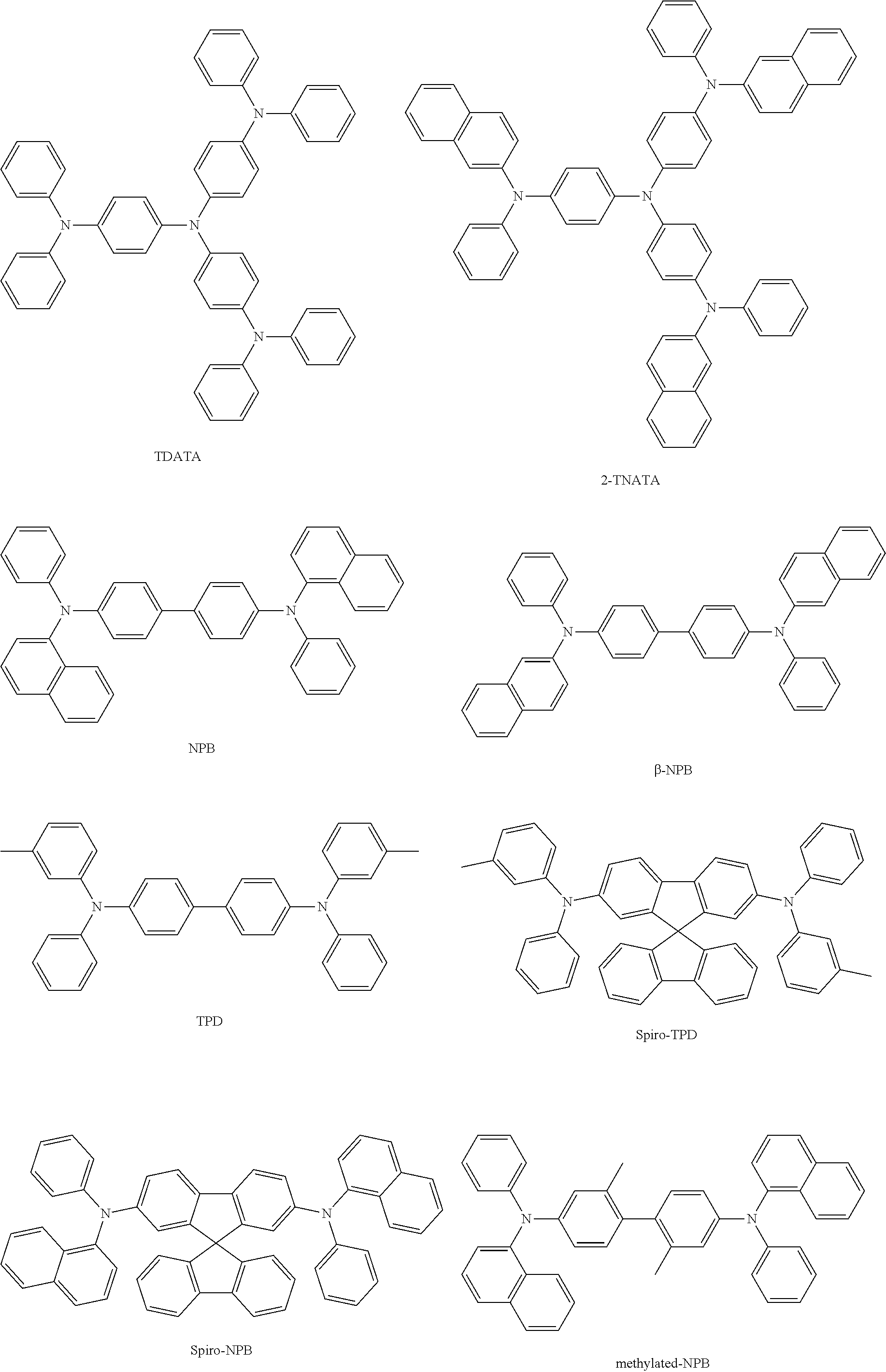

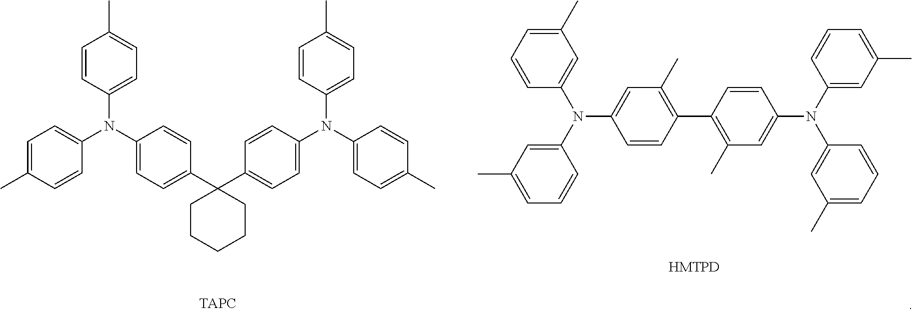

A thickness of the hole transport region may be in a range of about 50 Å (Angstroms) to about 10,000 Å, and in some embodiments, about 100 A to about 4,000 Å. When the hole transport region includes a hole injection layer, a hole transport layer, or a combination thereof, a thickness of the hole injection layer may be in a range of about 100 Å to about 9,000 Å, and in some embodiments, about 100 Å to about 1,000 Å, and a thickness of the hole transport layer may be in a range of about 50 Å to about 2,000 Å, and in some embodiments, about 100 Å to about 1,500 Å. When the thicknesses of the hole transport region, the hole injection layer, and the hole transport layer are within these ranges, excellent or suitable hole transport characteristics may be obtained without a substantial increase in driving voltage.

The emission auxiliary layer may increase light emission efficiency by compensating for an optical resonance distance according to the wavelength of light emitted by the emission layer. The electron blocking layer may prevent or reduce leakage of electrons to the hole transport region from the emission layer. Materials that may be included in the hole transport region may also be included in the emission auxiliary layer and the electron blocking layer.

p-Dopant

In one or more embodiments, the hole transport region may include a charge generating material as well as the aforementioned materials to improve conductive properties of the hole transport region. The charge generating material may be substantially homogeneously or non-homogeneously dispersed (for example, as a single layer including (e.g., consisting of) the charge generating material) in the hole transport region.

The charge generating material may include, for example, a p-dopant.

In some embodiments, a lowest unoccupied molecular orbital (LUMO) energy level of the p-dopant may be −3.5 eV or less.

In some embodiments, the p-dopant may include a quinone derivative, a compound containing a cyano group, a compound containing element EL1 and element EL2, or any combination thereof.

Non-limiting examples of the quinone derivative may include tetracyanoquinodimethane (TCNQ), 2,3,5,6-tetrafluoro-7,7,8,8-tetracyanoquinodimethane (F4-TCNQ), and/or the like.

Non-limiting examples of the compound containing a cyano group include dipyrazino[2,3-f: 2′,3′-h] quinoxaline-2,3,6,7,10,11-hexacarbonitrile (HAT-CN), a compound represented by Formula 221, and/or the like:

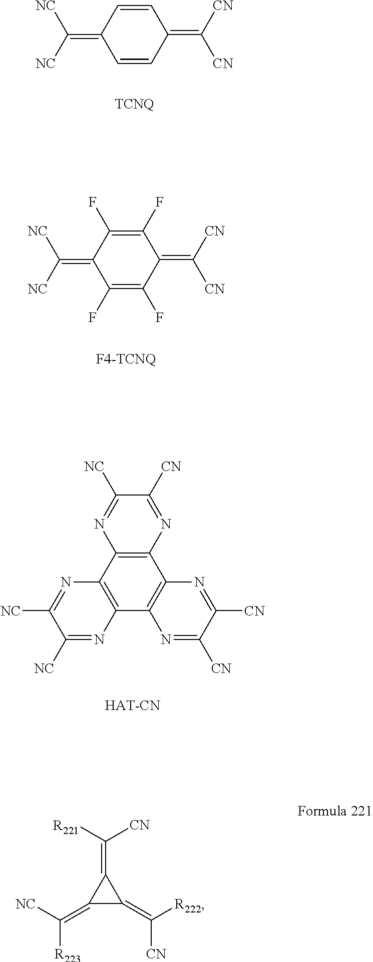

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
at least one selected from among R₂₂₁ to R₂₂₃ may each independently be: a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or a combination thereof, and element EL2 may be non-metal, a metalloid, or a combination thereof.

Non-limiting examples of the metal may include: an alkali metal (e.g., lithium (Li), sodium (Na), potassium (K), rubidium (Rb), cesium (Cs), and/or the like); an alkaline earth metal (e.g., beryllium (Be), magnesium (Mg), calcium (Ca), strontium (Sr), barium (Ba), and/or the like); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); a post-transition metal (e.g., zinc (Zn), indium (In), tin (Sn), and/or the like); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like); and/or the like.

Non-limiting examples of the metalloid may include silicon (Si), antimony (Sb), tellurium (Te), and/or the like.

Non-limiting examples of the non-metal may include oxygen (O), halogen (e.g., F, Cl, Br, I, and/or the like), and/or the like.

For example, the compound containing element EL1 and element EL2 may include a metal oxide, a metal halide (e.g., a metal fluoride, a metal chloride, a metal bromide, a metal iodide, and/or the like), a metalloid halide (e.g., a metalloid fluoride, a metalloid chloride, a metalloid bromide, a metalloid iodide, and/or the like), a metal telluride, or any combination thereof.

Non-limiting examples of the metal oxide may include a tungsten oxide (e.g., WO, W₂O₃, WO₂, WO₃, or W₂O₅), a vanadium oxide (e.g., VO, V₂O₃, VO₂, or V₂O₅), a molybdenum oxide (MoO, Mo₂O₃, MoO₂, MoO₃, or Mo₂O₅), a rhenium oxide (e.g., ReO₃), and/or the like.

Non-limiting examples of the metal halide may include alkali metal halide, alkaline earth metal halide, transition metal halide, post-transition metal halide, lanthanide metal halide, and/or the like.

Non-limiting examples of the alkali metal halide may include LiF, NaF, KF, RbF, CsF, LiCI, NaCl, KCl, RbCI, CsCl, LiBr, NaBr, KBr, RbBr, CsBr, Lil, Nal, KI, Rbl, Csl, and/or the like.

Non-limiting examples of the alkaline earth metal halide may include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, Bel₂, Mgl₂, Cal₂, Srl₂, Bal₂, and/or the like.

Non-limiting examples of the transition metal halide may include a titanium halide (e.g., TiF₄, TiCl₄, TiBr₄, or Til₄), a zirconium halide (e.g., ZrF₄, ZrCl₄, ZrBr₄, or Zrl₄), a hafnium halide (e.g., HfF₄, HfCl₄, HfBr₄, or Hfl₄), a vanadium halide (e.g., VF₃, VCl₃, VBr₃, or Vl₃), a niobium halide (e.g., NbF₃, NbCl₃, NbBr₃, or Nbl₃), a tantalum halide (e.g., TaF₃, TaCl₃, TaBr₃, or Tal₃), a chromium halide (e.g., CrF₃, CrCl₃, CrBr₃, or Crl₃), a molybdenum halide (e.g., MoF₃, MoCl₃, MoBr₃, or Mol₃), a tungsten halide (e.g., WF₃, WCl₃, WBr₃, or Wl₃), a manganese halide (e.g., MnF₂, MnCl₂, MnBr₂, or Mnl₂), a technetium halide (e.g., TcF₂, TcCl₂, TcBr₂, or Tcl₂), a rhenium halide (e.g., ReF₂, ReCl₂, ReBr₂, or Rel₂), a ferrous halide (e.g., FeF₂, FeCl₂, FeBr₂, or Fel₂), a ruthenium halide (e.g., RuF₂, RuCl₂, RuBr₂, or Rul₂), an osmium halide (e.g., OsF₂, OsCl₂, OsBr₂, or Osl₂), a cobalt halide (e.g., CoF₂, COCl₂, CoBr₂, or COl₂), a rhodium halide (e.g., RhF₂, RhCl₂, RhBr₂, or Rhl₂), an iridium halide (e.g., IrF₂, IrCl₂, IrBr₂, or Irl₂), a nickel halide (e.g., NiF₂, NiCl₂, NiBr₂, or Nil₂), a palladium halide (e.g., PdF₂, PdCl₂, PdBr₂, or Pdl₂), a platinum halide (e.g., PtF₂, PtCl₂, PtBr₂, or Ptl₂), a cuprous halide (e.g., CuF, CuCl, CuBr, or Cul), a silver halide (e.g., AgF, AgCl, AgBr, or Agl), a gold halide (e.g., AuF, AuCI, AuBr, or Aul), and/or the like.

Non-limiting examples of the post-transition metal halide may include a zinc halide (e.g., ZnF₂, ZnCl₂, ZnBr₂, or Znl₂), an indium halide (e.g., Inl₃), a tin halide (e.g., Snl₂), and/or the like.

Non-limiting examples of the lanthanide metal halide may include YbF, YbF₂, YbF₃, SmF₃, YbCl, YbCl₂, YbCl₃, SmCl₃, YbBr, YbBr₂, YbBr₃, SmBr₃, Ybl, Ybl₂, Ybl₃, Sml₃, and/or the like.

Non-limiting examples of the metalloid halide may include an antimony halide (e.g., SbCl₅) and/or the like.

Non-limiting examples of the metal telluride may include an alkali metal telluride (e.g., Li₂Te, Na₂Te, K₂Te, Rb₂Te, or Cs₂Te), an alkaline earth metal telluride (e.g., BeTe, MgTe, CaTe, SrTe, or BaTe), a transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, or Au₂Te), a post-transition metal telluride (e.g., ZnTe), a lanthanide metal telluride (e.g., LaTe, CeTe, PrTe, NdTe, PmTe, EuTe, GdTe, TbTe, DyTe, HoTe, ErTe, TmTe, YbTe, or LuTe), and/or the like.

### Emission Layer in Interlayer 130

When the light-emitting device 10 is a full color light-emitting device, the emission layer may be patterned into a red emission layer, a green emission layer, and/or a blue emission layer, according to a sub-pixel. In one or more embodiments, the emission layer may have a stacked structure. The stacked structure may include two or more layers selected from a red emission layer, a green emission layer, and a blue emission layer. The two or more layers may be in direct contact with each other or may be separated from each other to emit white light (e.g., combined white light). In one or more embodiments, the emission layer may include two or more materials. The two or more materials may include a red light-emitting material, a green light-emitting material, and/or a blue light-emitting material. The two or more materials may be mixed with each other in a single layer to emit white light (e.g., combined white light).

In one or more embodiments, the emission layer may include a host and a dopant. The dopant may be a phosphorescent dopant, a fluorescent dopant, or any combination thereof.

An amount of the dopant in the emission layer may be in a range of about 0.01 parts to about 15 parts by weight based on 100 parts by weight of the host.

In one or more embodiments, the emission layer may further include a first host and a second host, and the first host may be an electron transporting host, and the second host may be a hole transporting host.

In one or more embodiments, the first host may include at least one azine moiety, and the second host may include at least one carbazole moiety.

In one or more embodiments, the first host may include a compound represented by Formula 5:

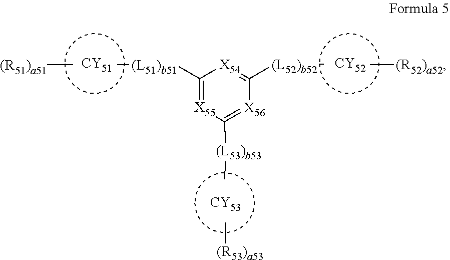

wherein, in Formula 5,
X₅₄ to X₅₆ may each independently be C(R₅₀), CH, or N, and at least one selected from among X₅₄ to X₅₆ may be N,
ring CY₅₁ to ring CY₅₃ may each independently include a C₅-C₆₀ carbocyclic group or a C₁-C₆₀ heterocyclic group,
L₅₁ to L₅₃ may each independently include a C₃-C₆₀ carbocyclic group unsubstituted or substituted with at least one R₁₀ₐ or a C₁-C₆₀ heterocyclic group unsubstituted or substituted with at least one R₁₀ₐ,
b51 to b53 may each independently be an integer from 0 to 3,
when b51 is 0, *-(L₅₁)_{b51}-*′ may be a single bond,
when b52 is 0, *-(L₅₂)_{b52}-*′ may be a single bond,
when b53 is 0, *-(L₅₃)_{b53}-*′ may be a single bond,
R₅₀ to R₅₃ may each be understood by referring to the description of R₁ provided herein,
a51 to a53 may each independently be an integer from 0 to 10.

In one or more embodiments, in Formula 5, ring CY₅₁ to ring CY₅₃ may each independently include i) a first ring, ii) a second ring, iii) a condensed ring in which at least two first rings are condensed, iv) a condensed ring in which at least two second rings are condensed, or v) a condensed ring in which at least one first ring is condensed with at least one second ring,
the first ring may include a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group, and
the second ring may include a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In some embodiments, in Formula 5, rings CY₅₁ to CY₅₃ may each independently include a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ.

In one or more embodiments, in Formula 5, L₅₁ to L₅₃ may each independently include a benzene group, a naphthalene group, an anthracene group, a phenanthrene group, a triphenylene group, a pyrene group, a chrysene group, a cyclopentadiene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an azafluorene group, an azacarbazole group, an azadibenzofuran group, an azadibenzothiophene group, an azadibenzosilole group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group.

In one or more embodiments, the first host may include at least one selected from compounds represented by Formulae ETH1 to ETH32:

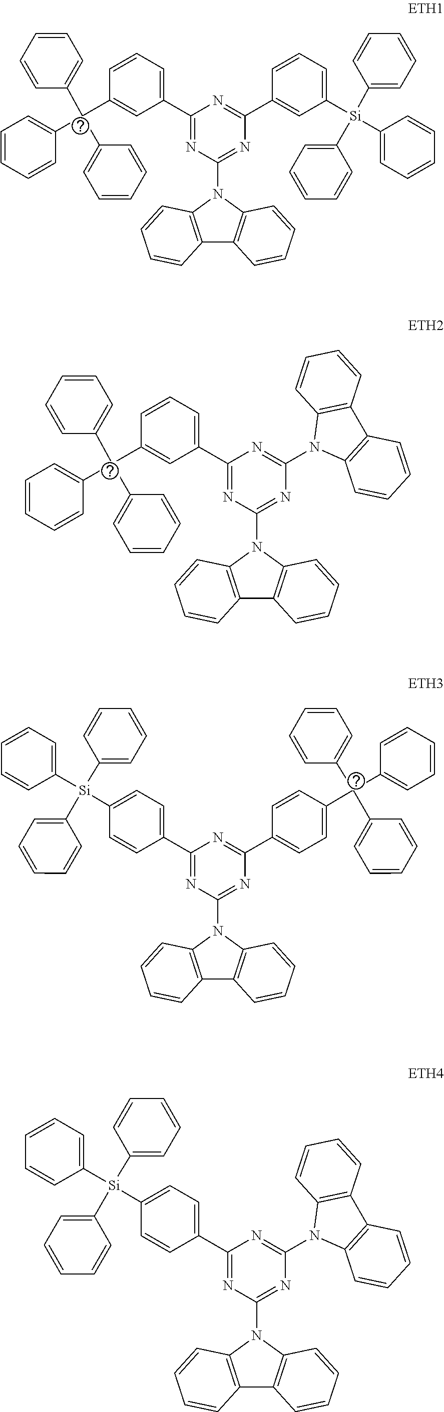

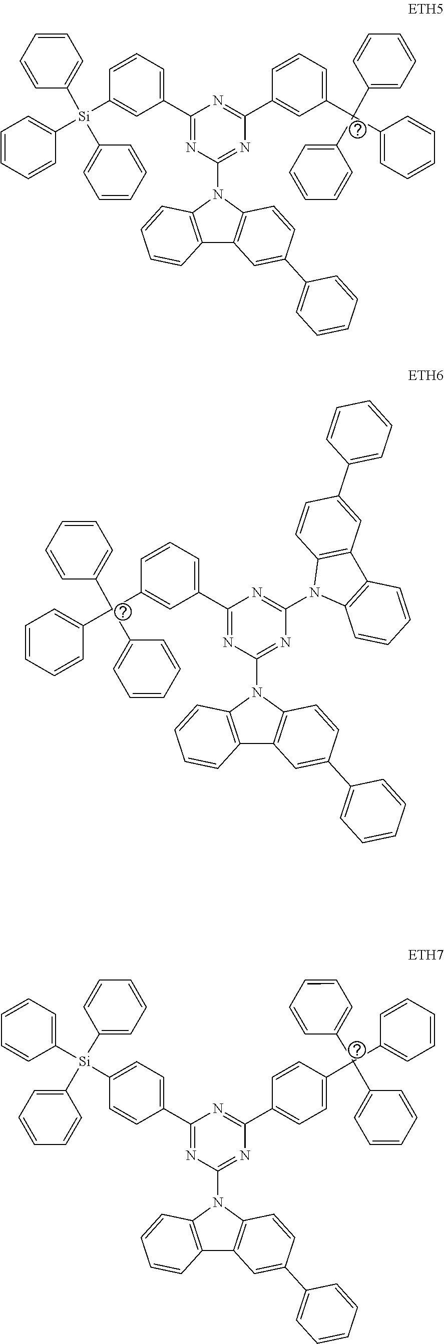

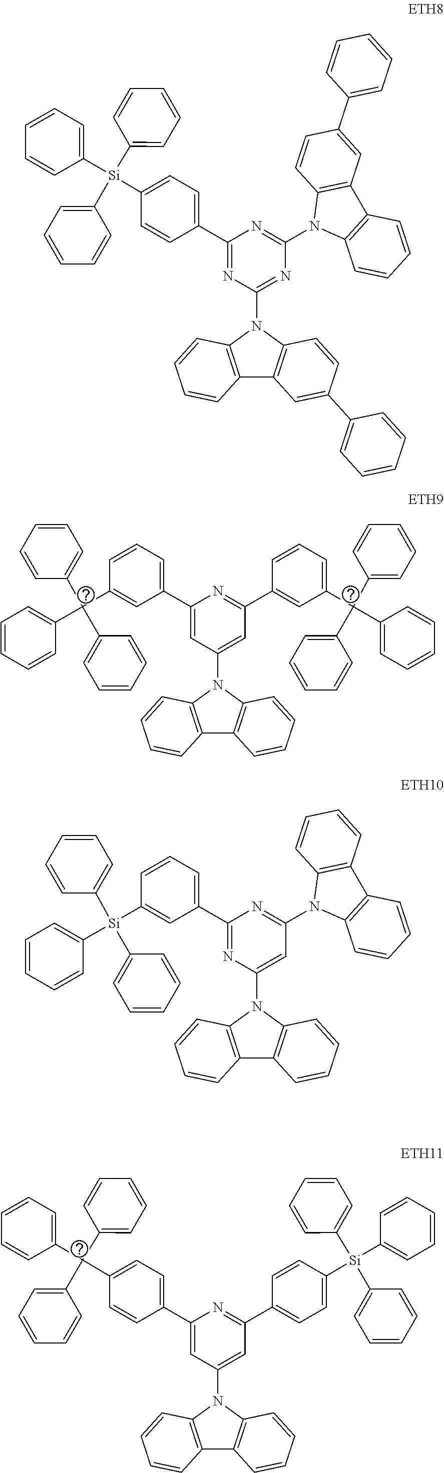

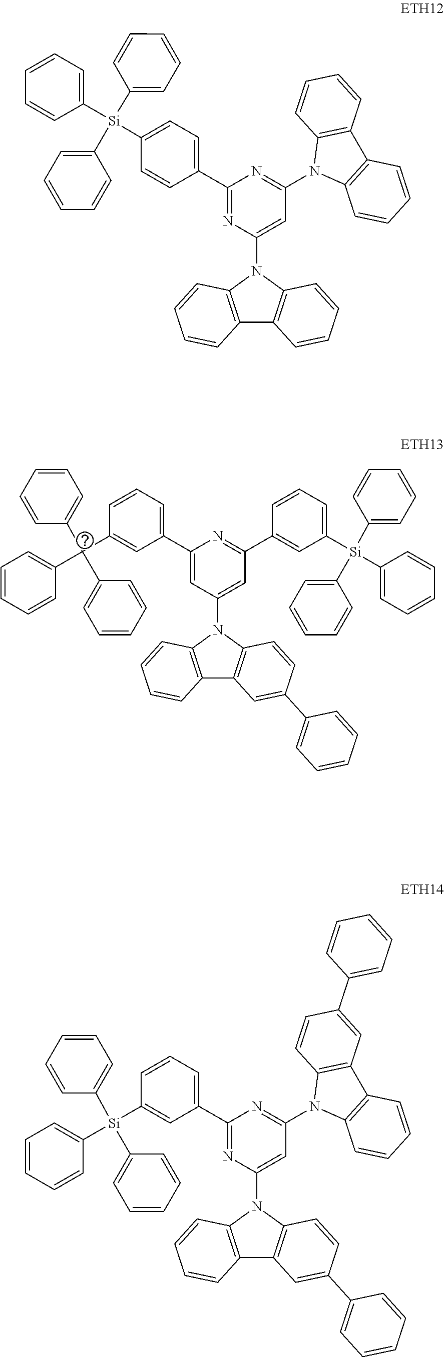

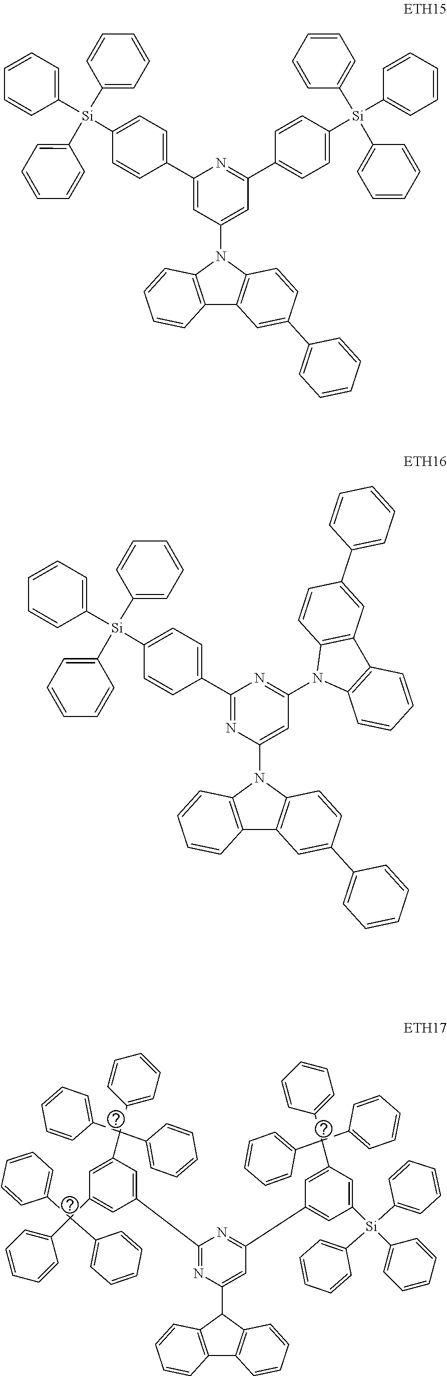 [SMILES: c1ccc(-c2ccc3c(c2)c2ccccc2n3-c2cc(-c3ccc([Si](c4ccccc4)(c4ccccc4)c4ccccc4)cc3)nc(-c3ccc([Si](c4ccccc4)(c4ccccc4)c4ccccc4)cc3)c2)cc1 ; c1ccc(-c2ccc3c(c2)c2ccccc2n3-c2cc(-n3c4ccccc4c4cc(-c5ccccc5)ccc43)nc(-c3ccc([Si](c4ccccc4)(c4ccccc4)c4ccccc4)cc3)n2)cc1 ; c1ccc(C(c2ccccc2)(c2ccccc2)c2cc(-c3nc(-c4cc(C(c5ccccc5)(c5ccccc5)c5ccccc5)cc([Si](c5ccccc5)(c5ccccc5)c5ccccc5)c4)cc(C4c5ccccc5-c5ccccc54)n3)cc(C(c3ccccc3)(c3ccccc3)c3ccccc3)c2)cc1]

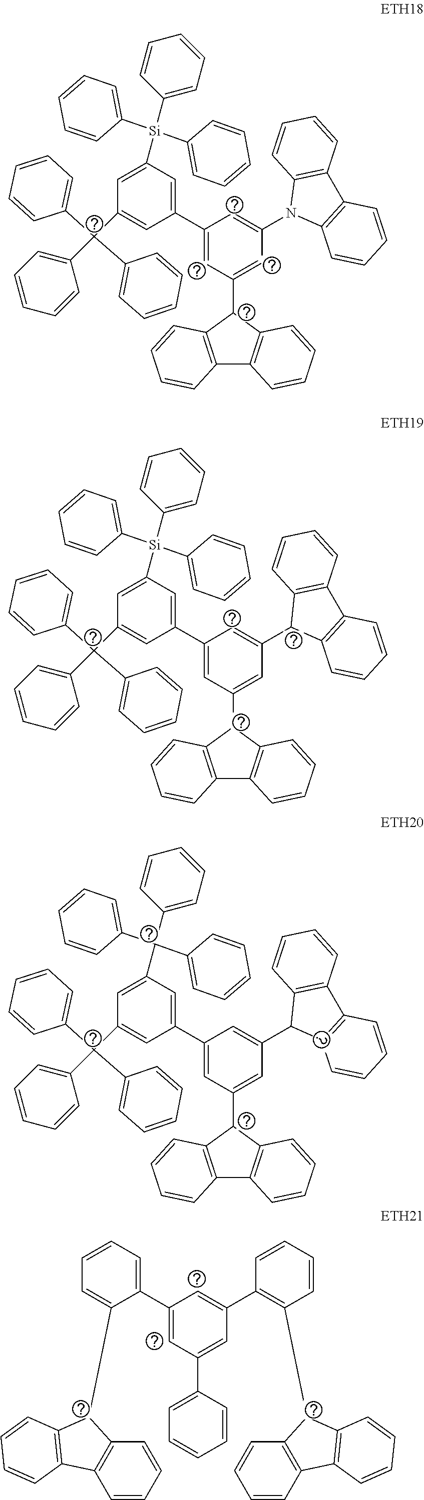

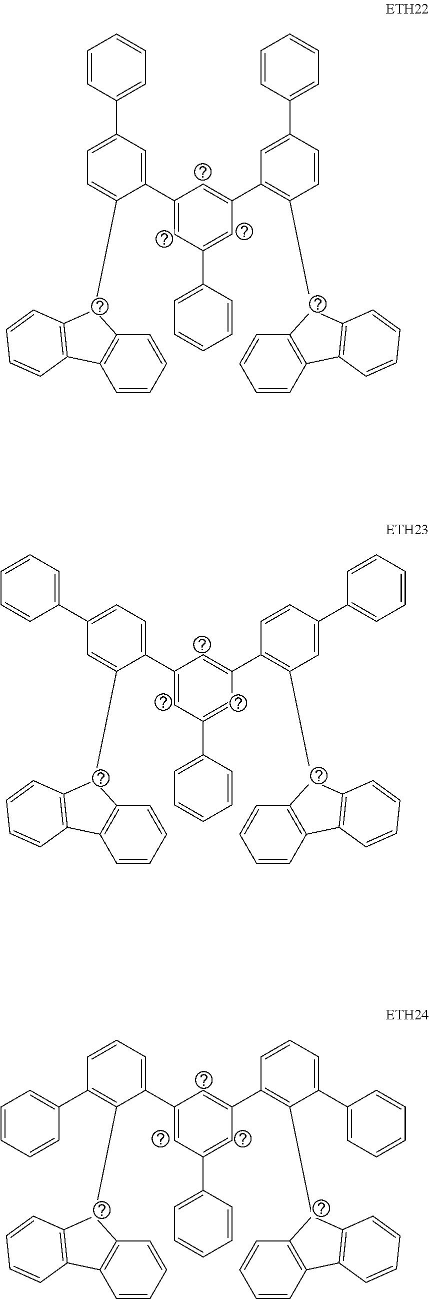

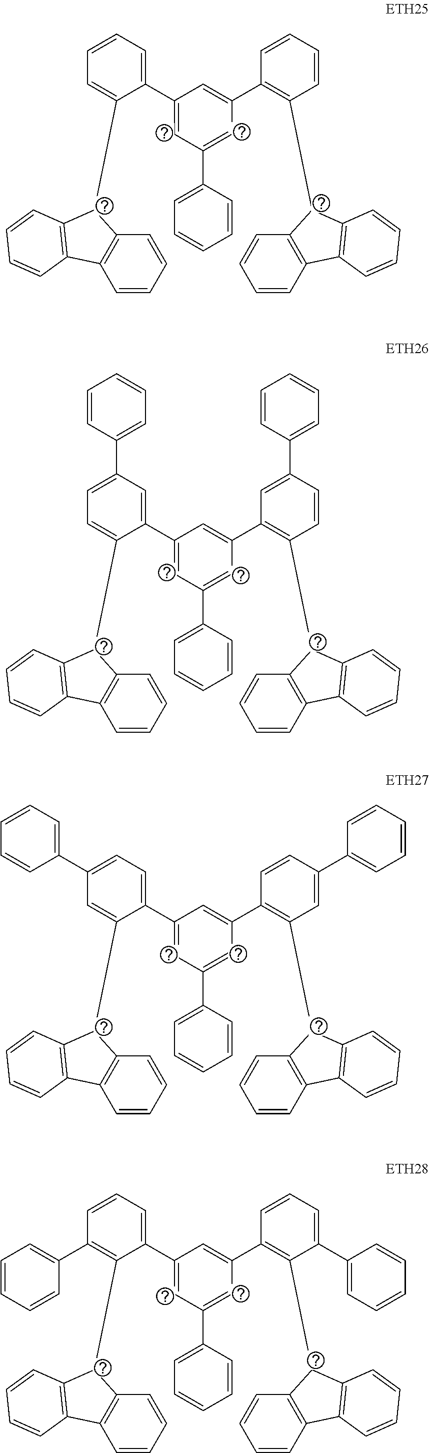

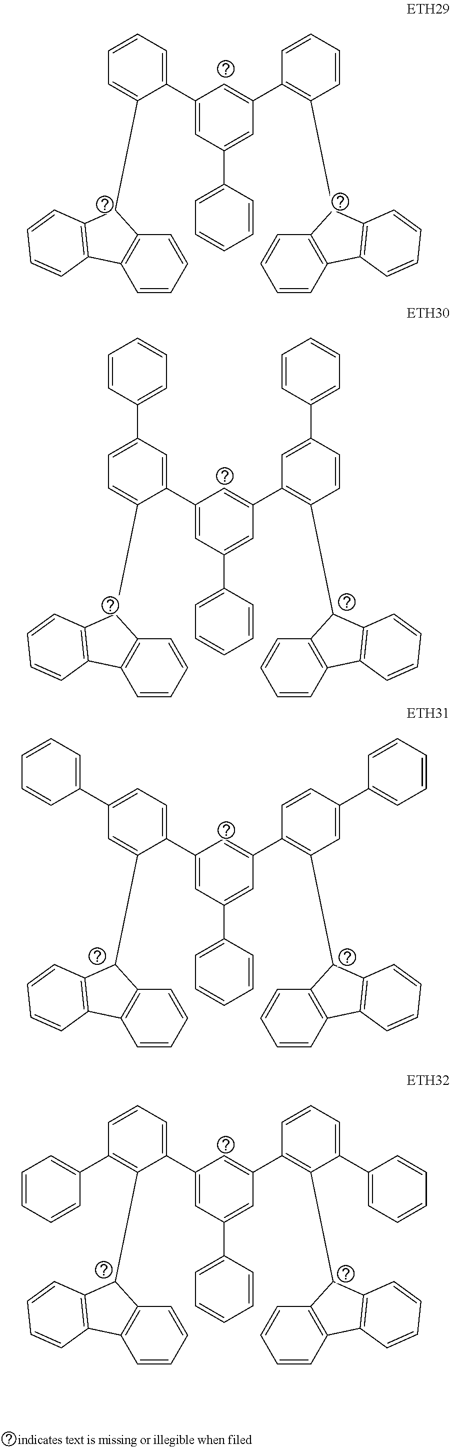

In one or more embodiments, the second host may include a compound including a group represented by Formula 7:

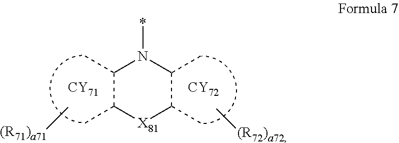

wherein, in Formula 7,
ring CY₇₁ and ring CY₇₂ may each independently include a C₅-C₆₀ carbocyclic group or a C₁-C₆₀ heterocyclic group,
X₈₁ may be a single bond, O, S, N(R₈₁), B(R₈₁), C(R₈₁ₐ)(R_{81b}), or Si(R₈₁ₐ)(R_{81b}),
R₇₁, R₇₂, R₈₁, R₈₁ₐ, and R_{81b} may each be understood by referring to the description of R₁ provided herein,
a71 and a72 may each independently be an integer from 0 to 10.

In one or more embodiments, in Formula 7, ring CY₇₁ and ring CY₇₂ may each independently include i) a first ring, ii) a second ring, iii) a condensed ring in which at least two first rings are condensed, iv) a condensed ring in which at least two second rings are condensed, or v) a condensed ring in which at least one first ring is condensed with at least one second ring,
the first ring may include a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group, and
the second ring may include a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In some embodiments, in Formula 7, rings CY₇₁ and CY₇₂ may each independently include a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluoren-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isooxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ.

In one or more embodiments, the second host may include a compound represented by one selected from Formulae 7-1 to 7-5:

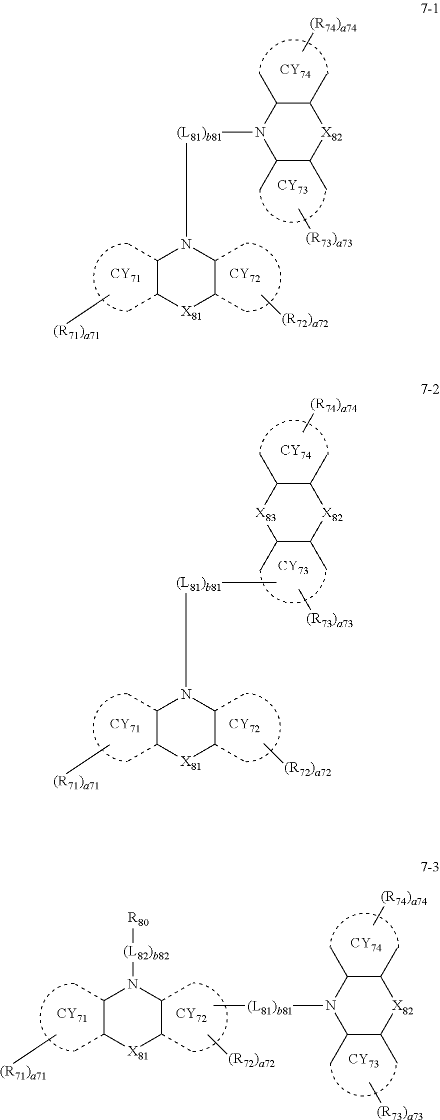

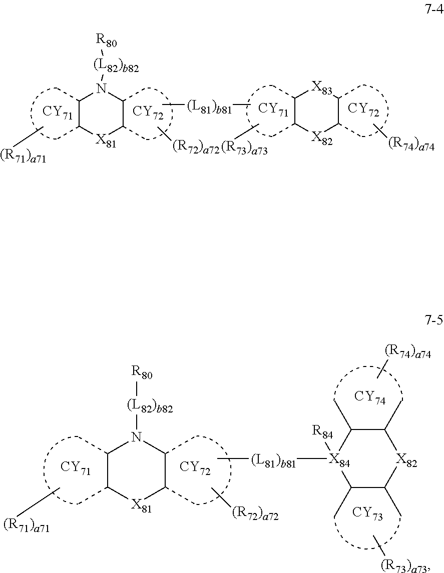

wherein, in Formulae 7-1 to 7-5,
ring CY₇₁, ring CY₇₂, X₈₁, R₇₁, R₇₂, a71, and a72 may respectively be understood by referring to the descriptions of ring CY₇₁, ring CY₇₂, X₈₁, R₇₁, R₇₂, a71, and a72 provided herein,
ring CY₇₃, ring CY₇₄, R₇₃, R₇₄, a73, and a74 may respectively be understood by referring to the descriptions of ring CY₇₁, ring CY₇₂, R₇₁, R₇₂, a71, and a72 provided herein,
L₈₁ and L₈₂ may each independently be selected from *—C(Q₄)(Q₅)-*′, *—Si(Q₄)(Q₅)-*′, a substituted or unsubstituted C₅-C₃₀ carbocyclic group, and a substituted or unsubstituted C₁-C₃₀ heterocyclic group, wherein Q₄ and Q₅ may each be understood by referring to the description of Q₁ provided herein,
b81 and b82 may each be an integer from 0 to 5, when b81 is 0, *-(L₈₁)_{b81}-*′ may be a single bond, when b81 is 2 or greater, at least two L₈₁(s) may be identical to or different from each other, when b82 is 0, *-(L₈₂)_{b82}-*′ may be a single bond, when b82 is 2 or greater, at least two L₈₂(s) may be identical to or different from each other,
X₈₂ may be a single bond, O, S, N(R₈₂), B(R₈₂), C(R₈₂ₐ)(R_{82b}), or Si(R₈₂ₐ)(R_{82b}),
X₈₃ may be a single bond, O, S, N(R₈₃), B(R₈₃), C(R₈₃ₐ)(R_{83b}), or Si(R₈₃ₐ)(R_{83b}),
X₈₂ and X₈₃ in Formulae 7-2 and 7-4 may not each be a single bond at the same time,
X₈₄ may be C or Si,
R₈₀, R₈₂, R₈₃, R₈₂ₐ, R_{82b}, R₈₃ₐ, R_{83b}, and R₈₄ may each be understood by referring to the description of R₈₁ provided herein, and
* and *′ each indicates a binding site to an adjacent atom.

In one or more embodiments,
in Formulae 7-1 and 7-2, a moiety represented by

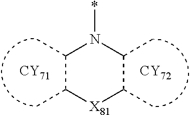

may be selected from groups represented by Formulae CY71-1(1) to CY71-1(8),
in Formulae 7-1 and 7-3, a moiety represented by

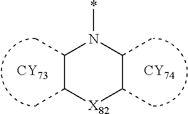

may be selected from groups represented by Formulae CY71-2(1) to CY71-2(8)
in Formulae 7-2 and 7-4, a moiety represented by

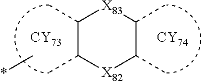

may be selected from groups represented by Formulae CY71-3(1) to CY71-3(32),
in Formulae 7-3 to 7-5, a moiety represented by

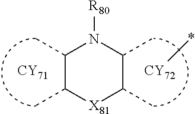

may be selected from groups represented by Formulae CY71-4(1) to CY71-4(32), and
in Formula 7-5, a moiety represented by

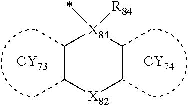

may be selected from groups represented by Formulae CY71-5(1) to CY71-5(8):

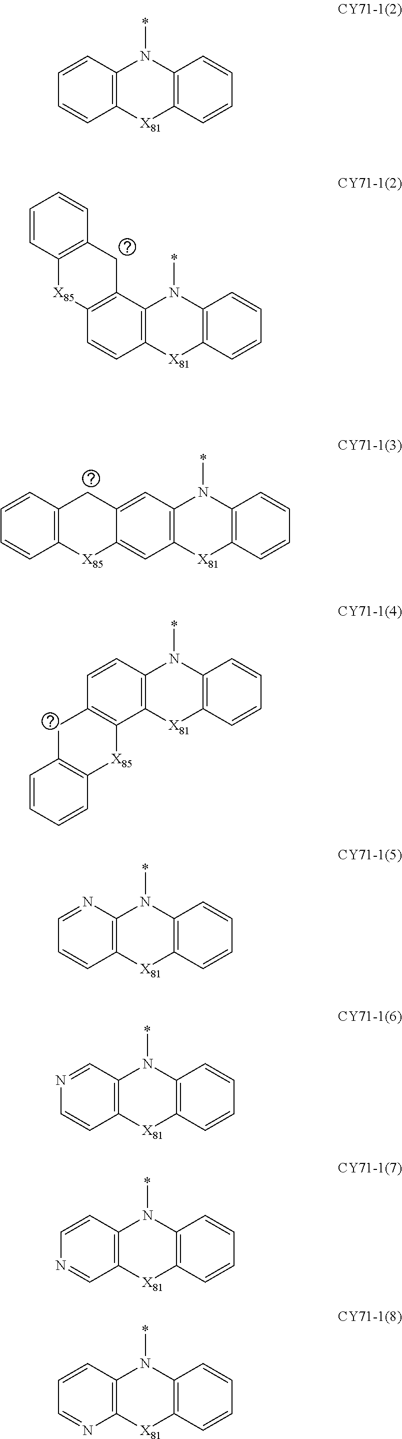

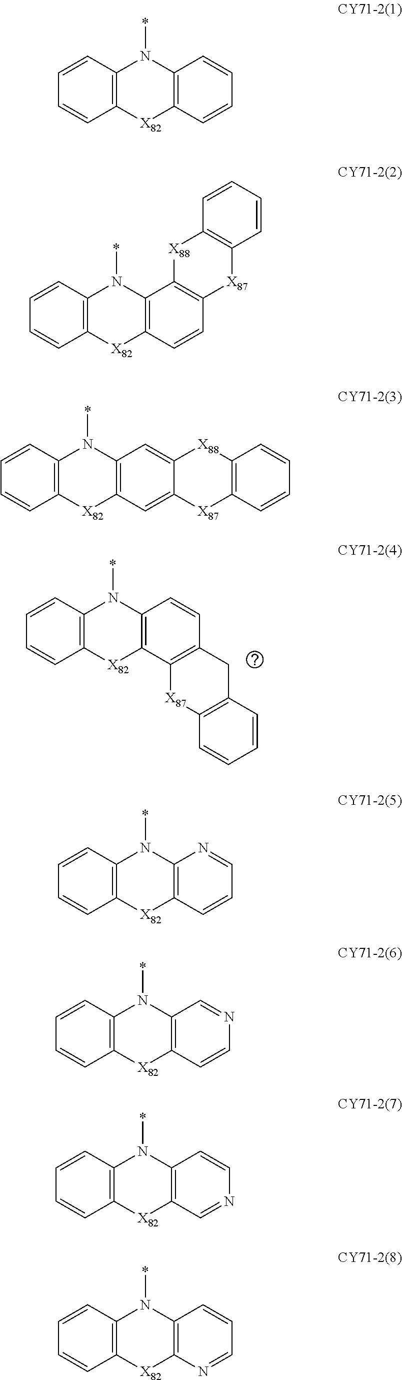

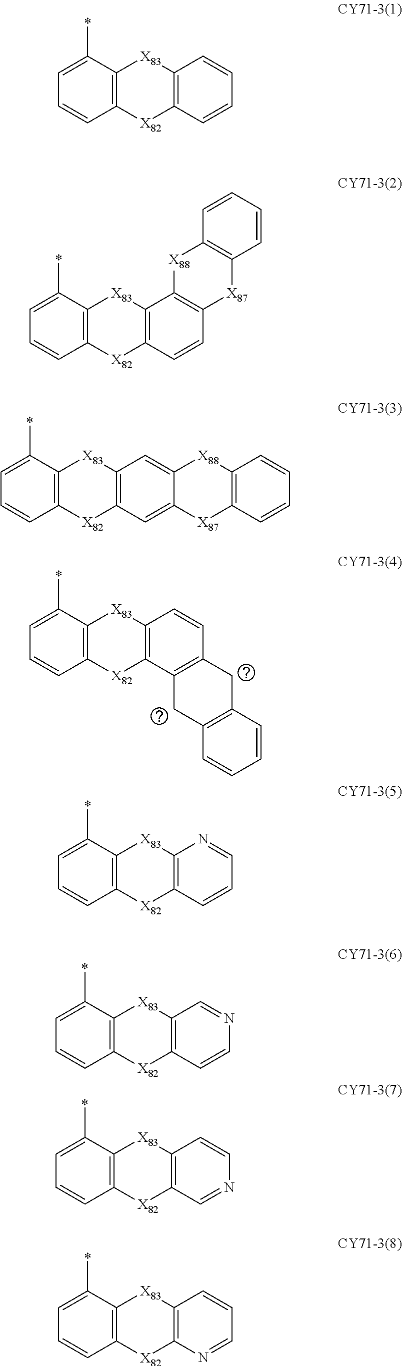

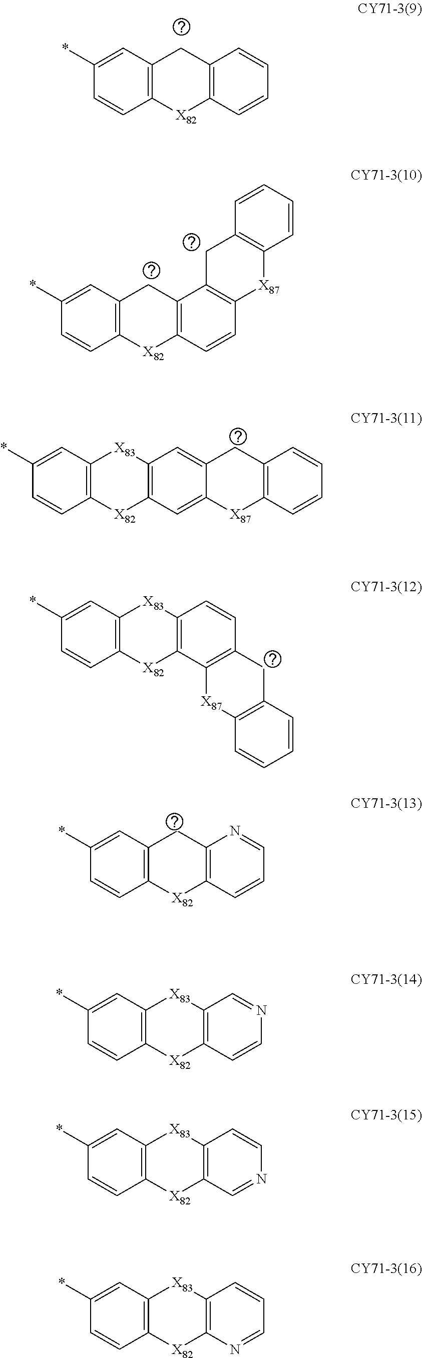

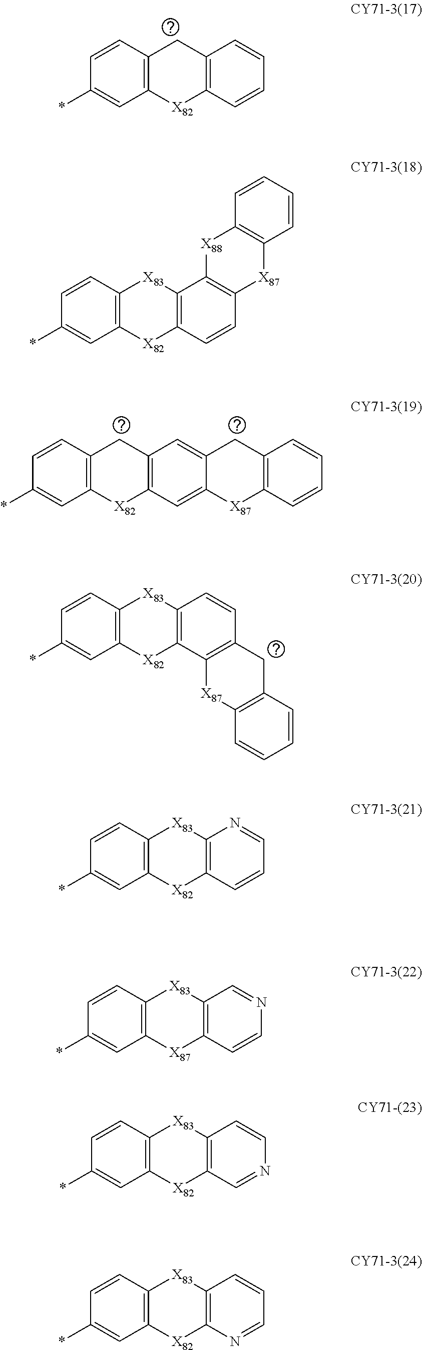

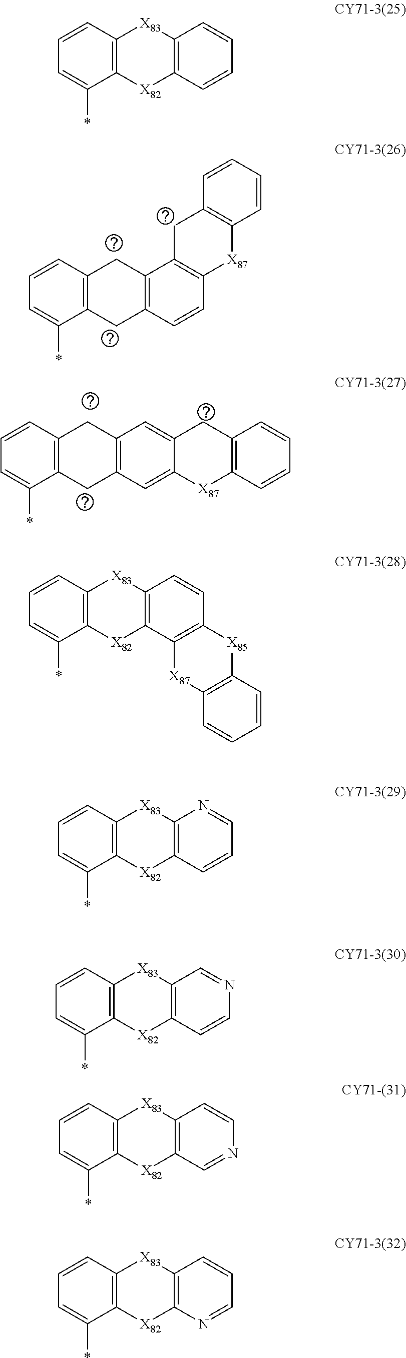

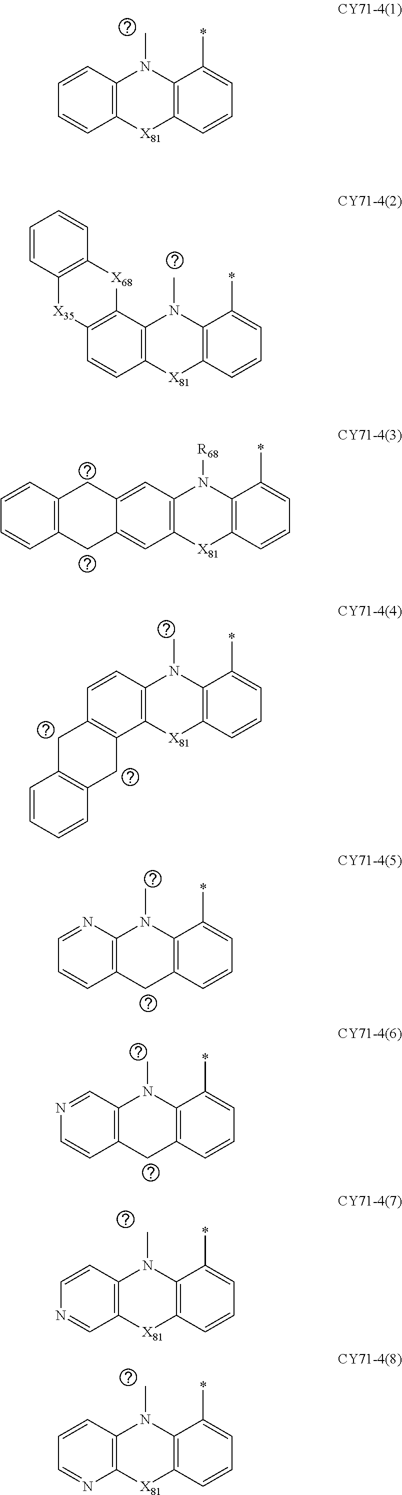

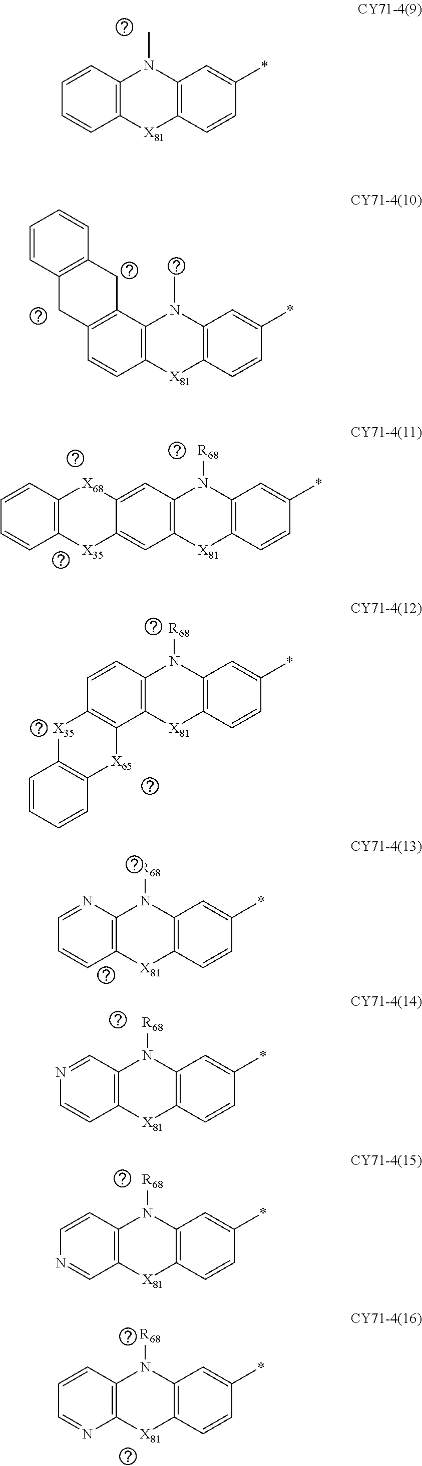

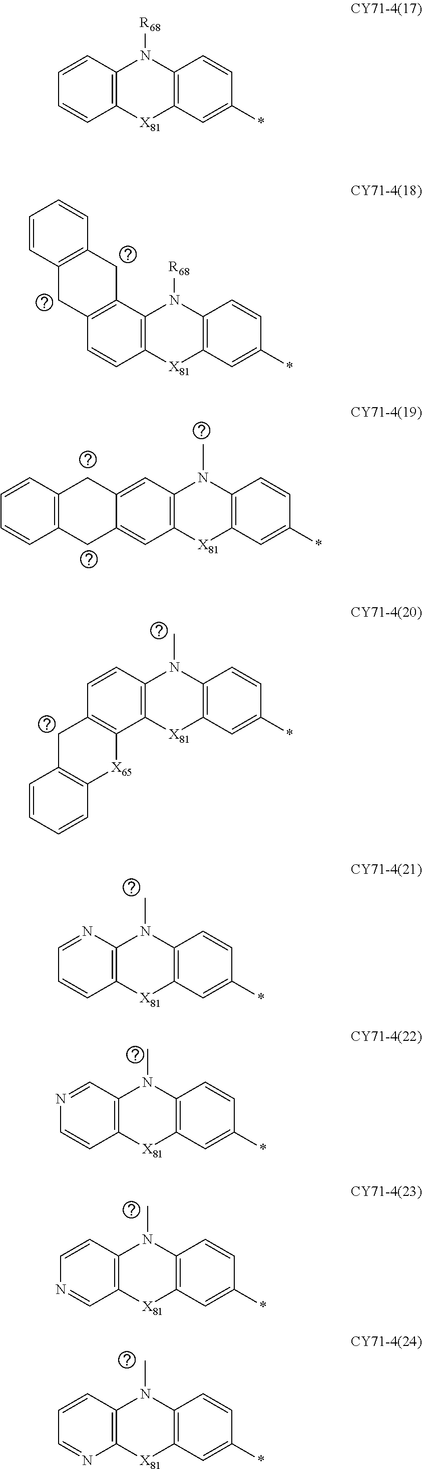

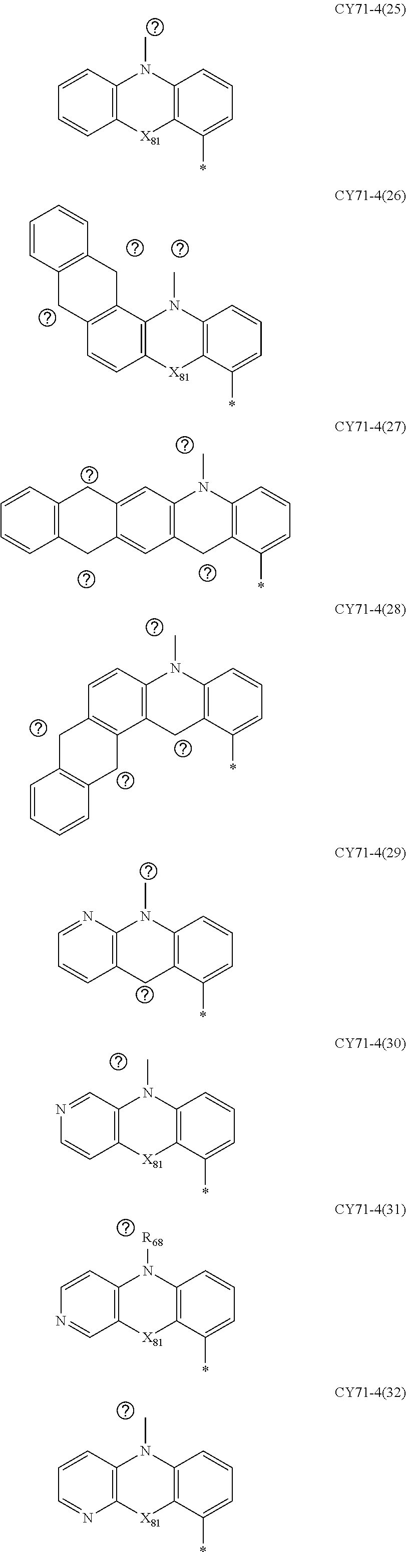

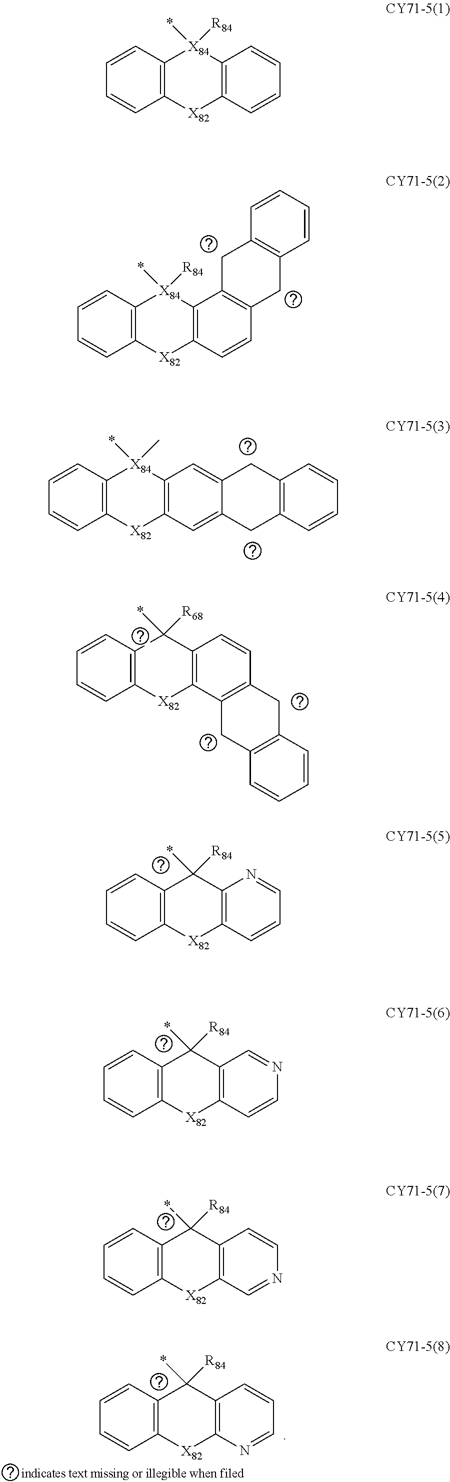

In Formulae CY71-1(1) to CY71-1(8), CY71-2(1) to CY71-2(8), CY71-3(1) to CY71-3(32), CY71-4(1) to CY71-4(32), and CY71-5(1) to CY71-5(8),
X₈₁ to X₈₄ and R₈₀ and R₈₄ may respectively be understood by referring to the descriptions of X₈₁ to X₈₄ and R₈₀ and R₈₄ provided herein,
X₈₅ may be a single bond, O, S, N(R₈₅), B(R₈₅), C(R₈₅ₐ)(R_{85b}), or Si(R₈₅ₐ) (R_{85b}),
X₈₆ may be a single bond, O, S, N(R₈₆), B(R₈₆), C(R₈₆ₐ)(R_{86b}), or Si(R₈₆ₐ)(R_{86b}),
in Formulae CY71-1(1) to CY71-1(8) and CY71-4(1) to CY71-4(32), X₈₅ and X₈₆ may not each be a single bond at the same time,
X₈₇ may be a single bond, O, S, N(R₈₇), B(R₈₇), C(R₈₇ₐ)(R_{87b}), or Si(R₈₇ₐ)(R_{87b}),
X₈₈ may be a single bond, O, S, N(R₈₈), B(R₈₈), C(R₈₈ₐ)(R_{88b}), or Si(R₈₈ₐ)(R_{88b}),
in Formulae CY71-2(1) to CY71-2(8), CY71-3(1) to CY71-3(32), and CY71-5(1) to CY71-5(8), X₈₇ and X₈₈ may not each be a single bond at the same time,
R₈₅ to R₈₈, R₈₅ₐ, R_{85b}, R₈₆ₐ, R_{86b}, R₈₇ₐ, R_{87b}, R₈₈ₐ, and R_{88b} may each be understood by referring to the description of R₈₁ provided herein.

In one or more embodiments, the second host may include at least one selected from compounds represented by Formulae HTH1 to HTH40.

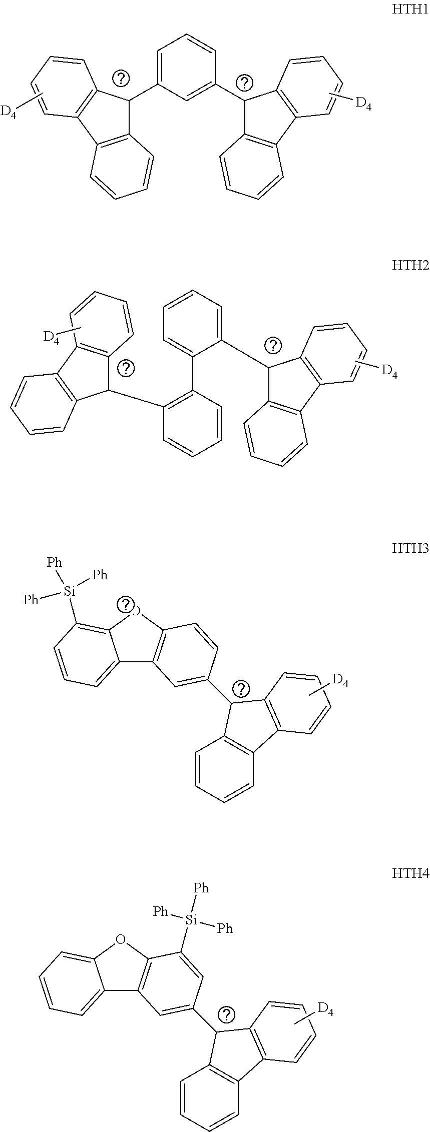

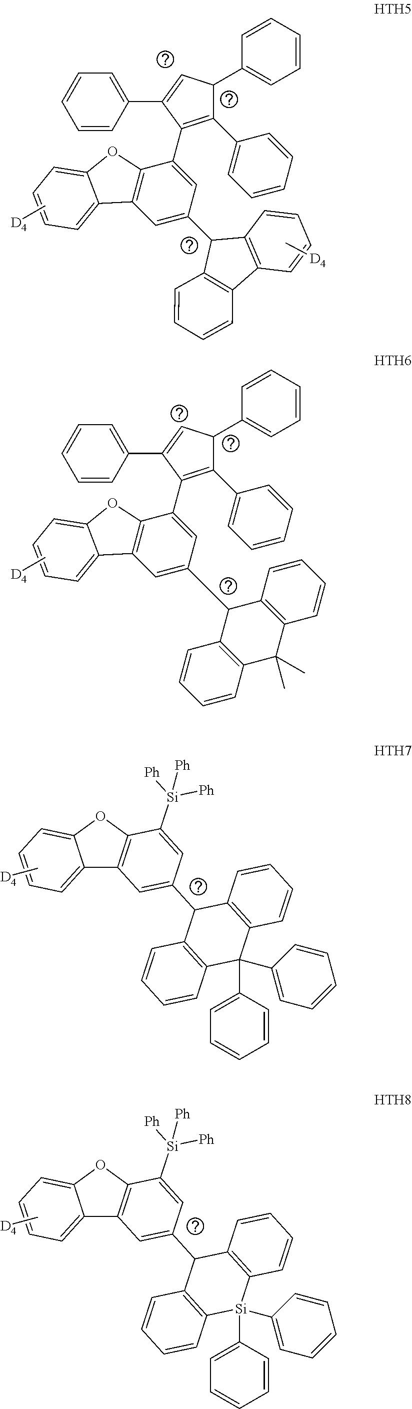

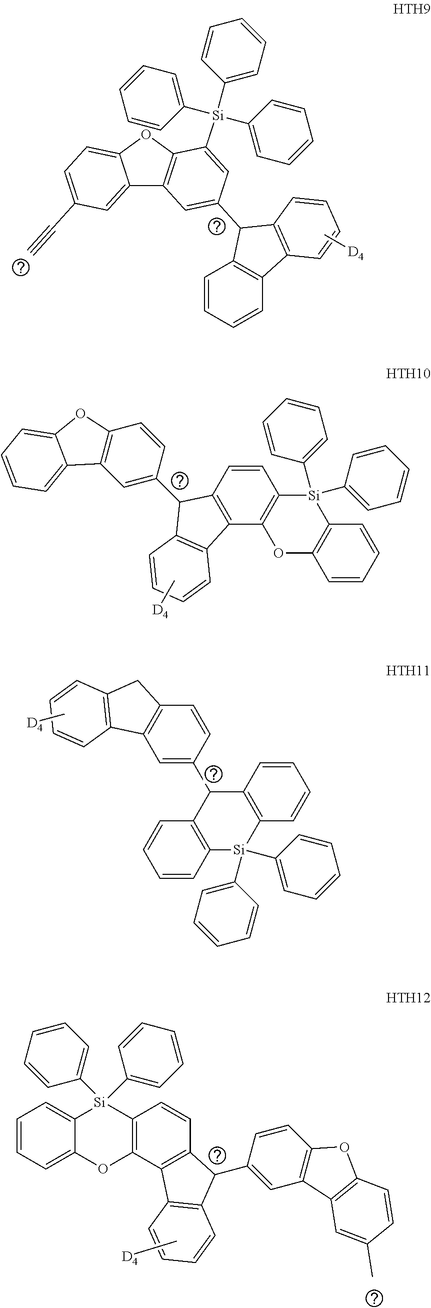

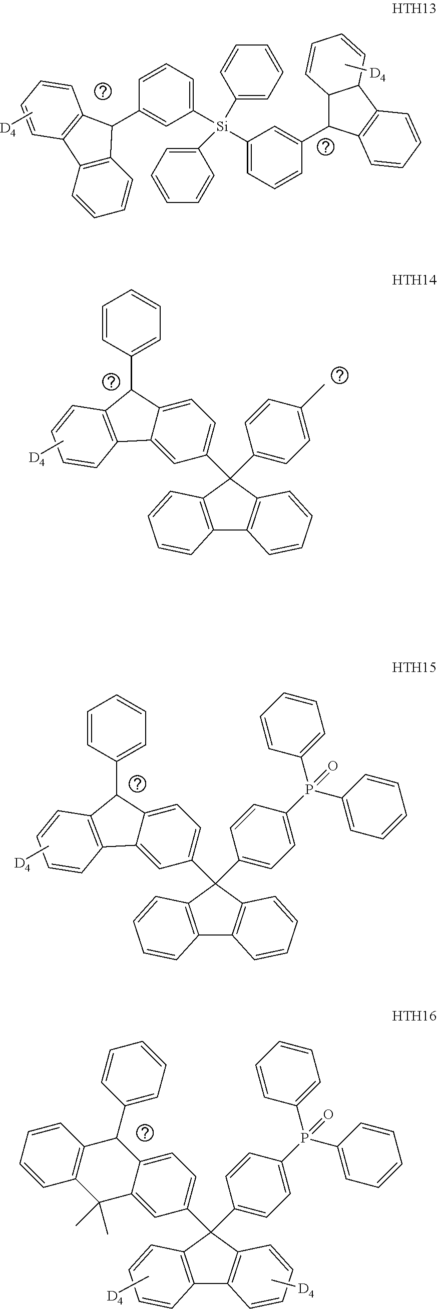

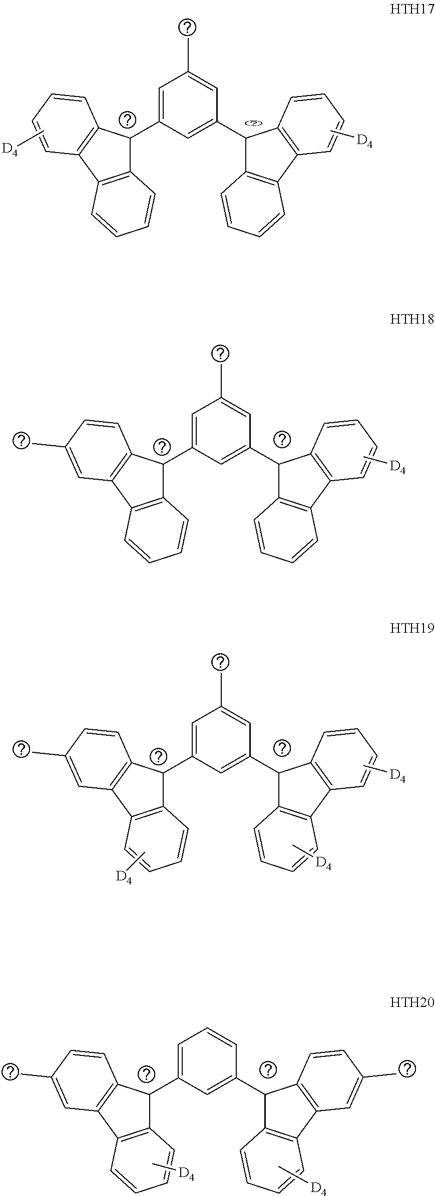

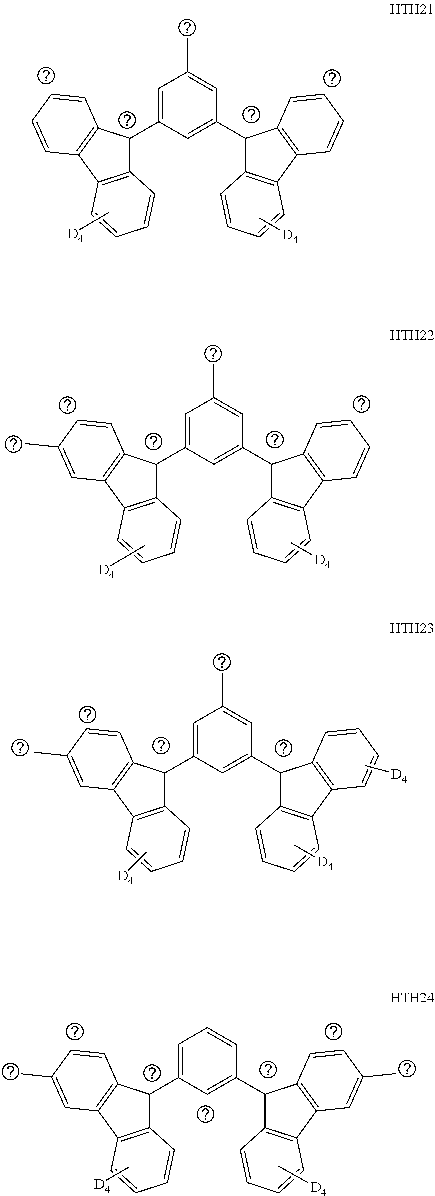

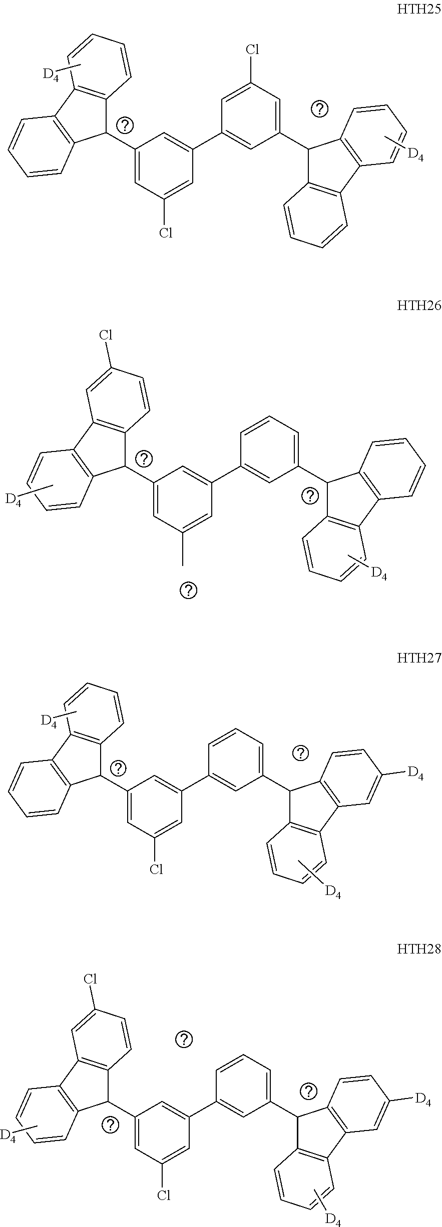

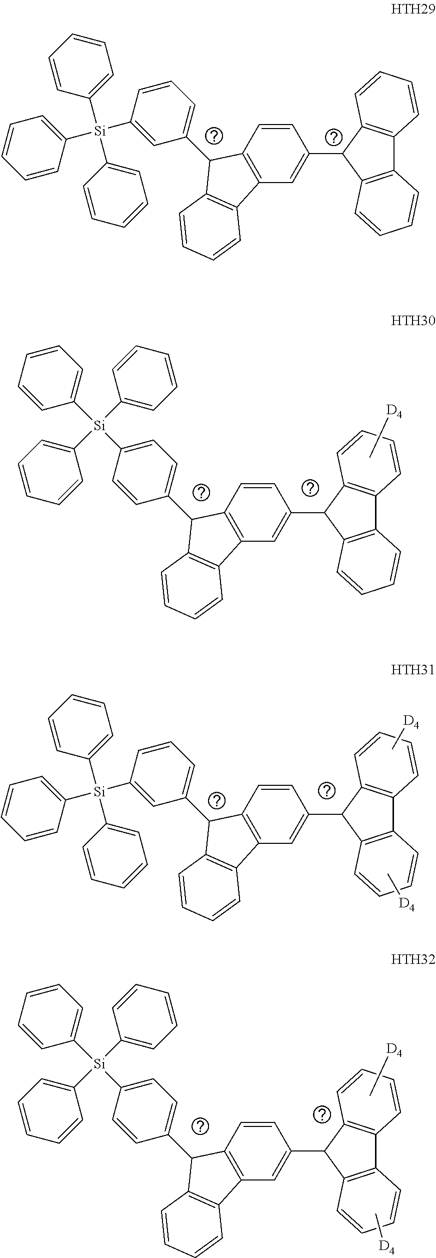

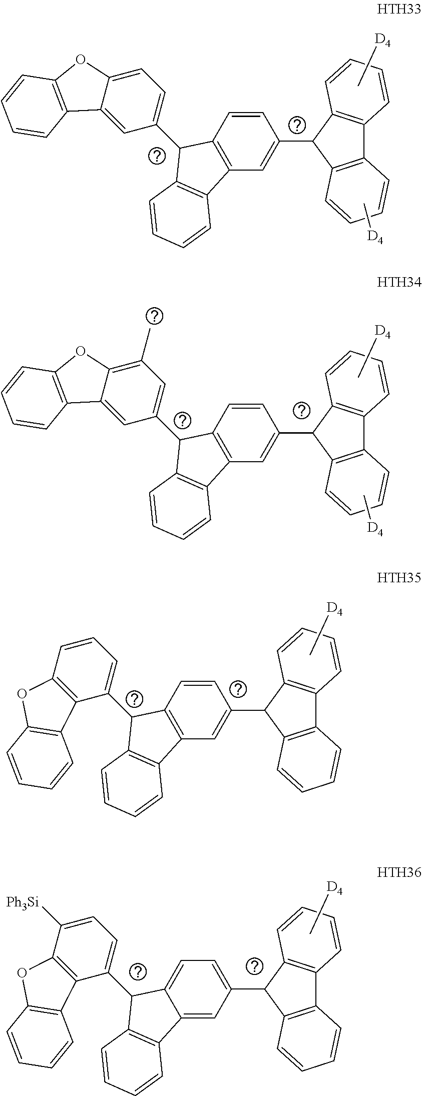

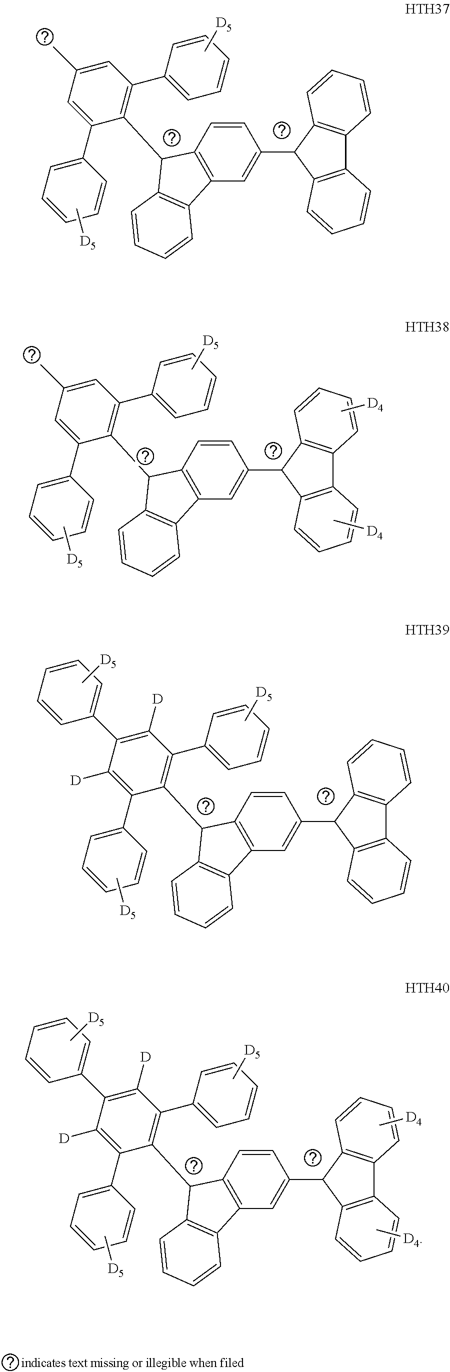

In a light-emitting device according to one or more embodiments, the first host and the second host may form an exciplex, an organometallic compound and the first host or the organometallic compound and the second host may not form an exciplex.

In some embodiments, the emission layer may include quantum dots.

In some embodiments, the emission layer may include a delayed fluorescence material. The delayed fluorescence material may serve as a host or a dopant in the emission layer.

A thickness of the emission layer may be in a range of about 100 Å to about 1,000 Å, and in some embodiments, about 200 Å to about 600 Å. When the thickness of the emission layer is within these ranges, improved luminescence characteristics may be obtained without a substantial increase in driving voltage.

### Host

In one or more embodiments, the host may include a compound represented by Formula 301:

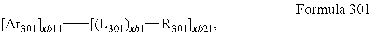

wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ may each be understood by referring to the description of Q₁ provided herein.

In some embodiments, when xb11 in Formula 301 is 2 or greater, at least two Ar₃₀₁(s) may be bound via a single bond.

In some embodiments, the host may include a compound represented by Formula 301-1, a compound represented by Formula 301-2, or any combination thereof:

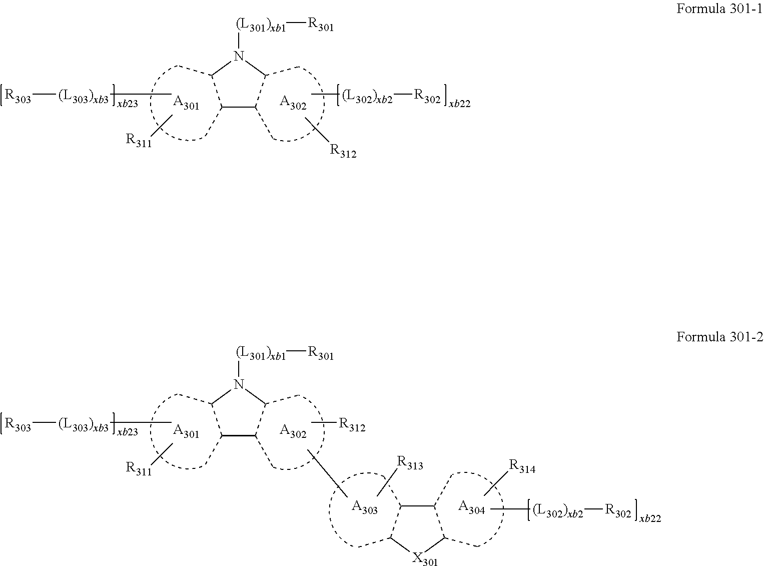

wherein, in Formulae 301-1 to 301-2,
ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ may respectively be understood by referring to the descriptions of L₃₀₁, xb1, and R₃₀₁ provided herein,
L₃₀₂ to L₃₀₄ may each be understood by referring to the description of L₃₀₁ provided herein,
xb2 to xb4 may each be understood by referring to the description of xb1 provided herein, and
R₃₀₂ to R₃₀₅ and R₃₁₁ to R₃₁₄ may each be understood by referring to the description of R₃₀₁ provided herein.

In some embodiments, the host may include an alkaline earth-metal complex, a post-transitional metal complex, or any combination thereof. For example, in some embodiments, the host may include a Be complex (e.g., Compound H55), an Mg complex, a Zn complex, or any combination thereof.

In some embodiments, the host may include one selected from among Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl) benzene (TCP), and/or any combination thereof:

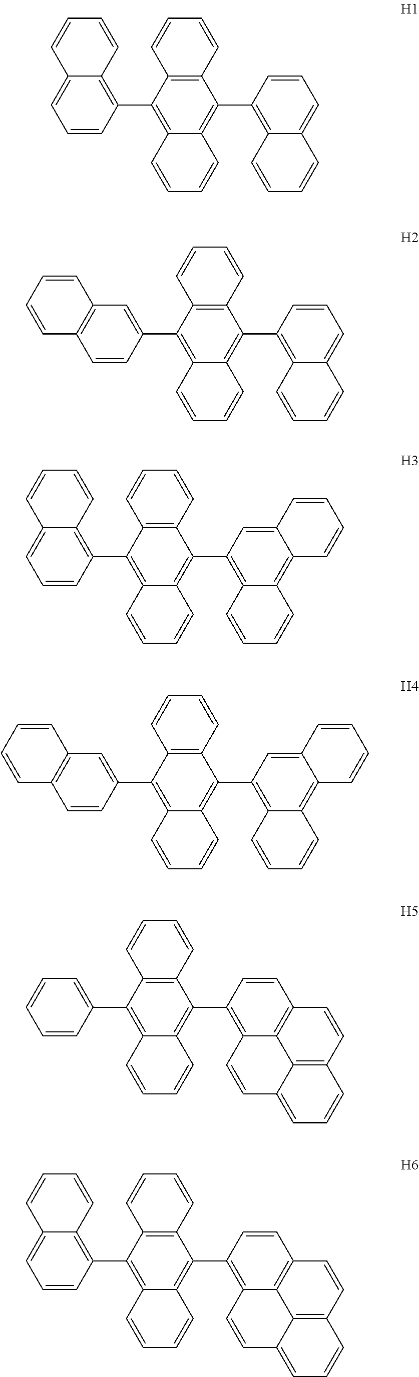

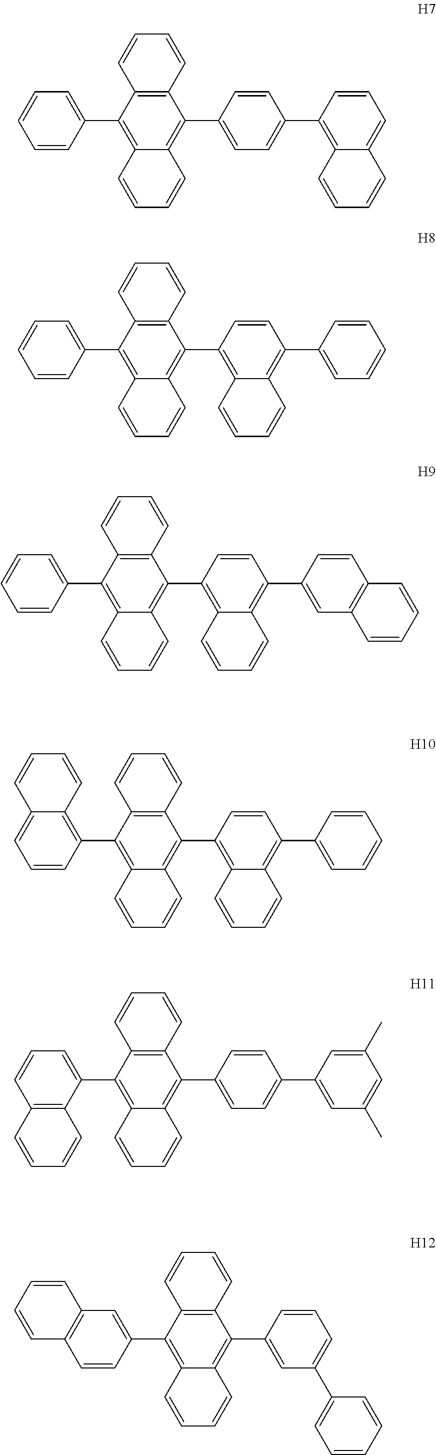

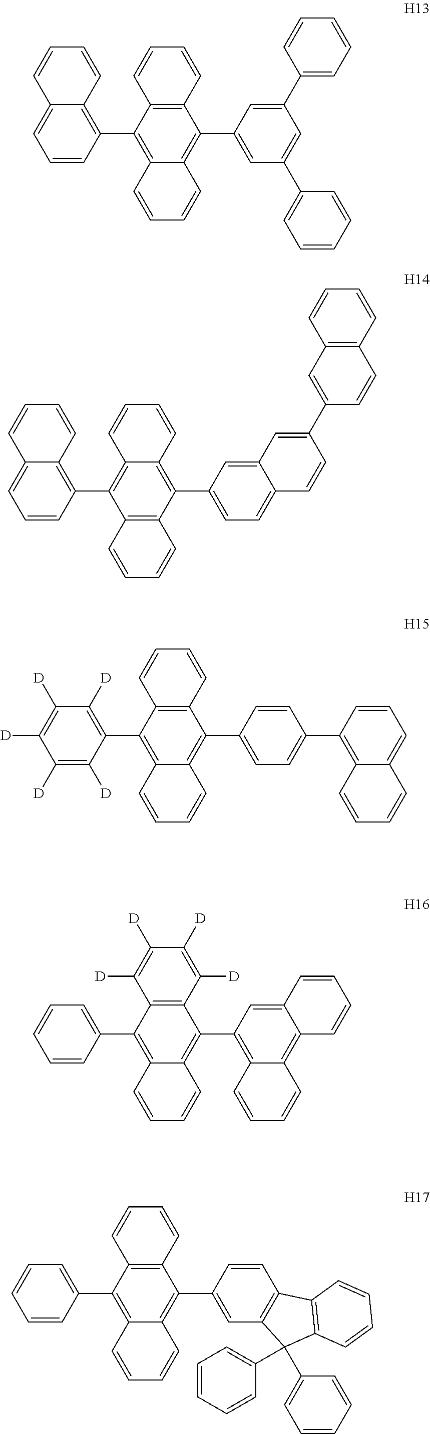

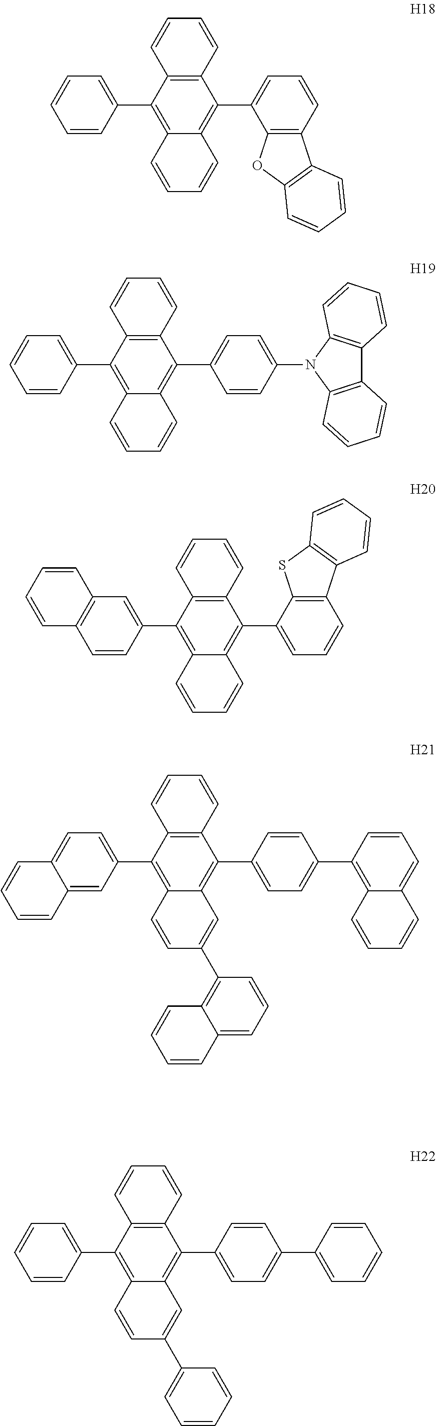

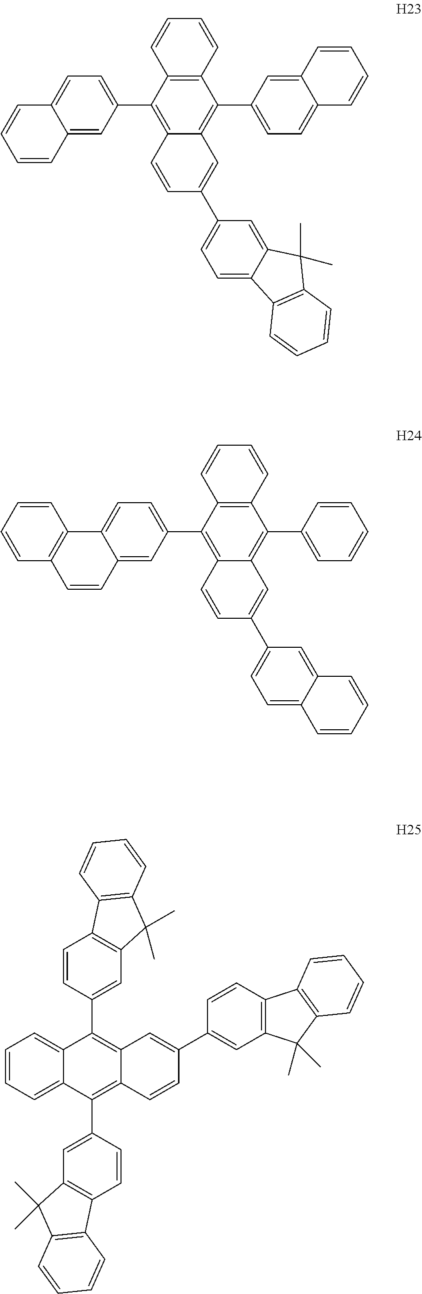

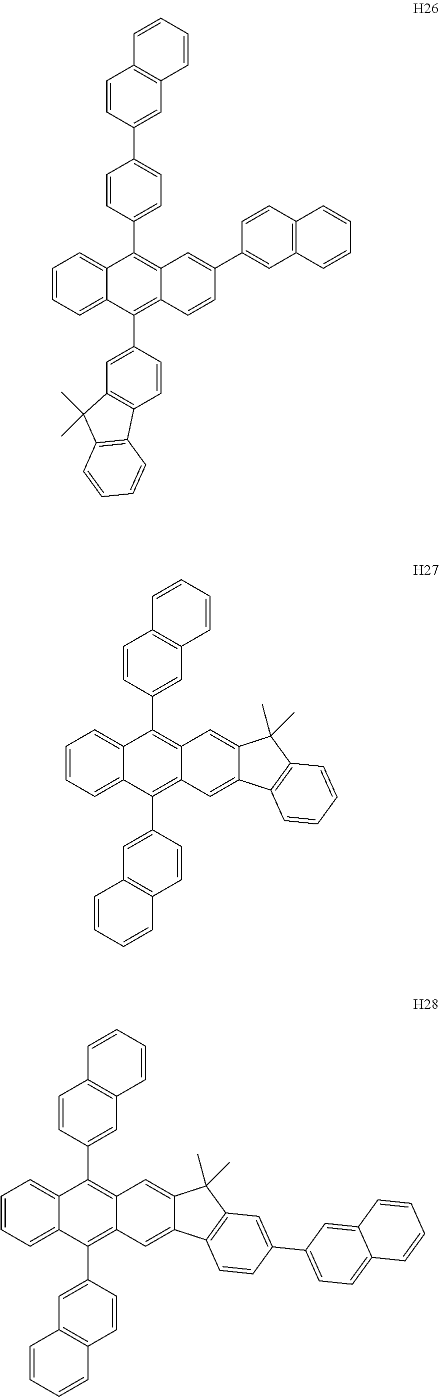

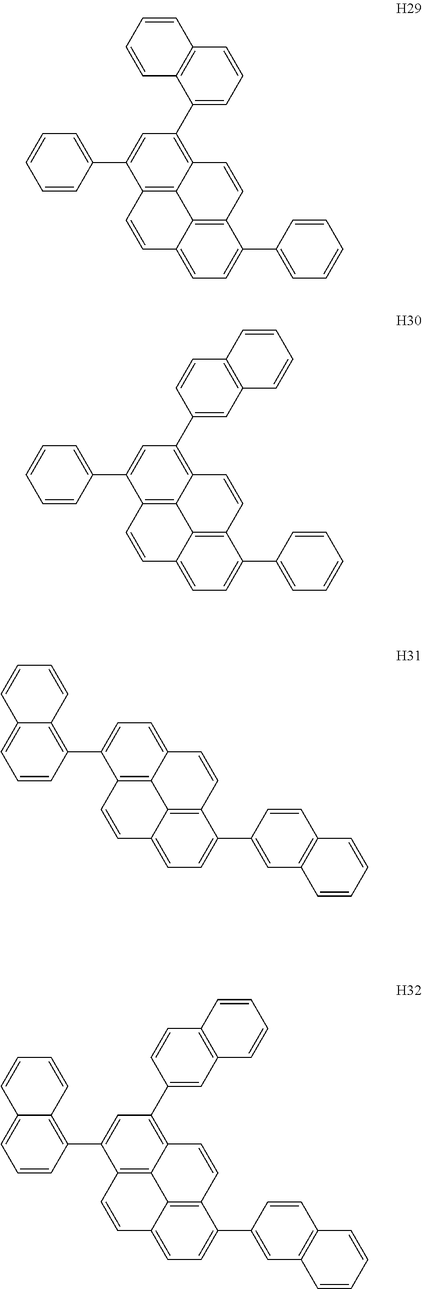

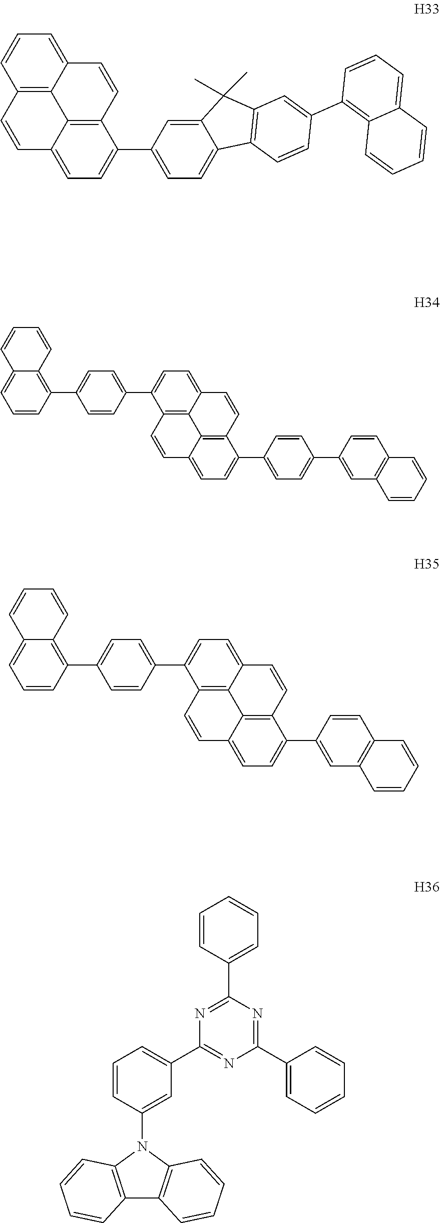

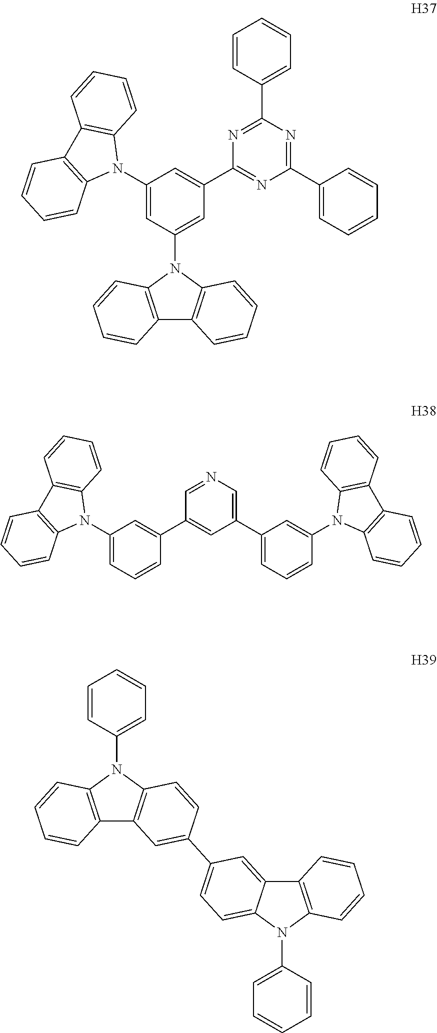 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

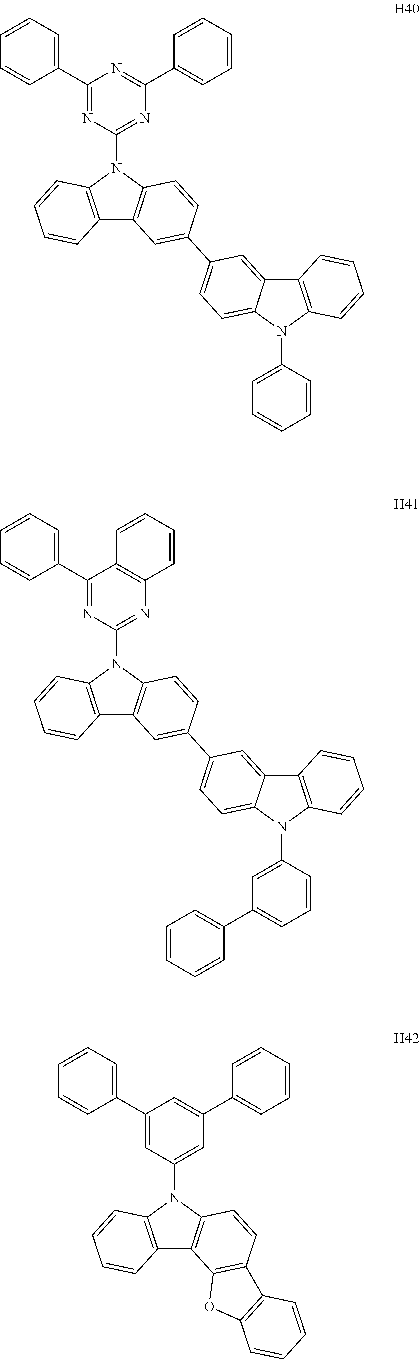 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

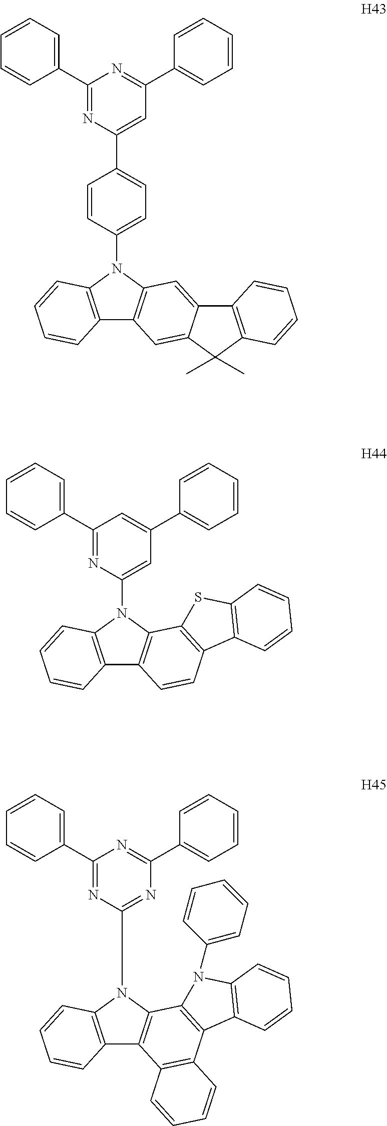 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1]

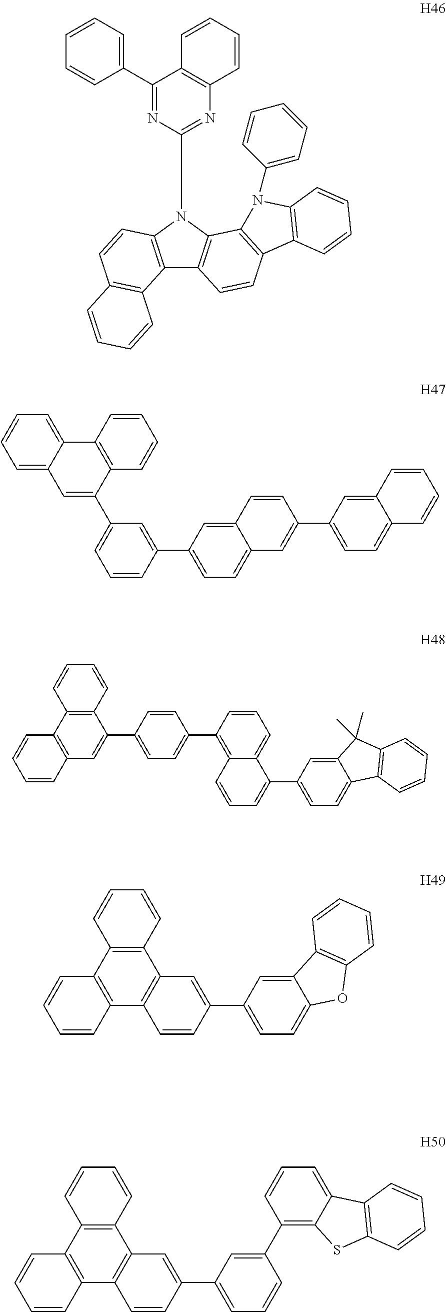

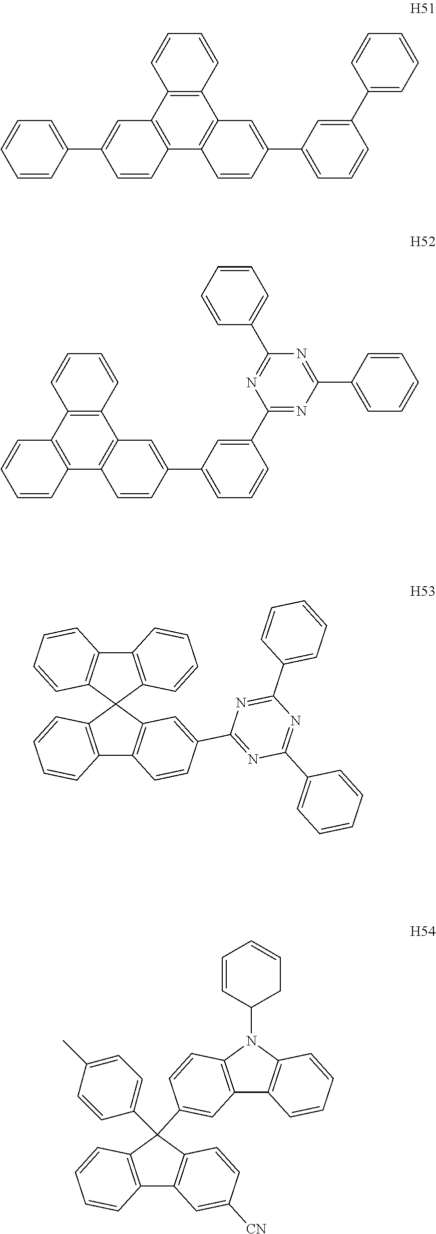

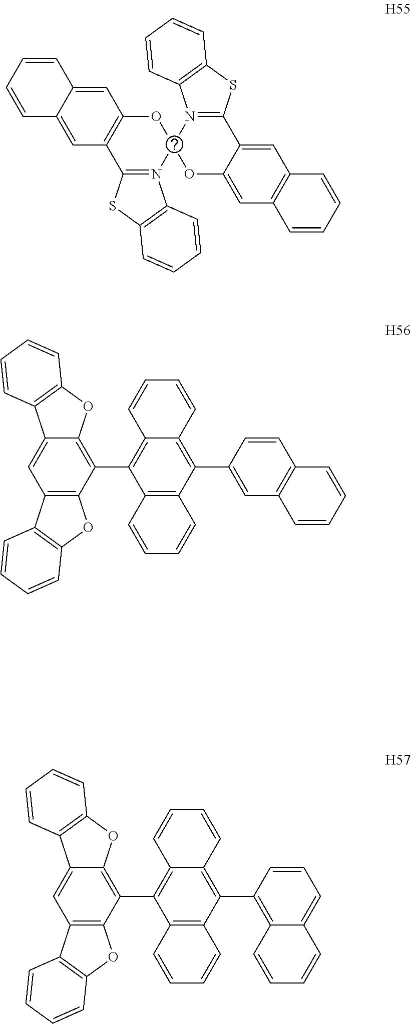

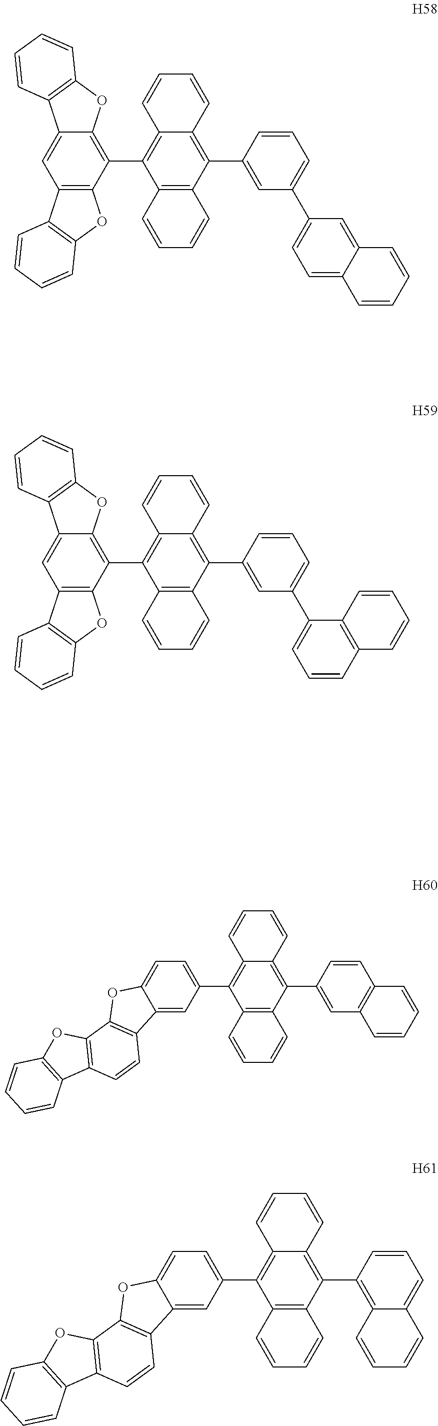 [SMILES: c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1]

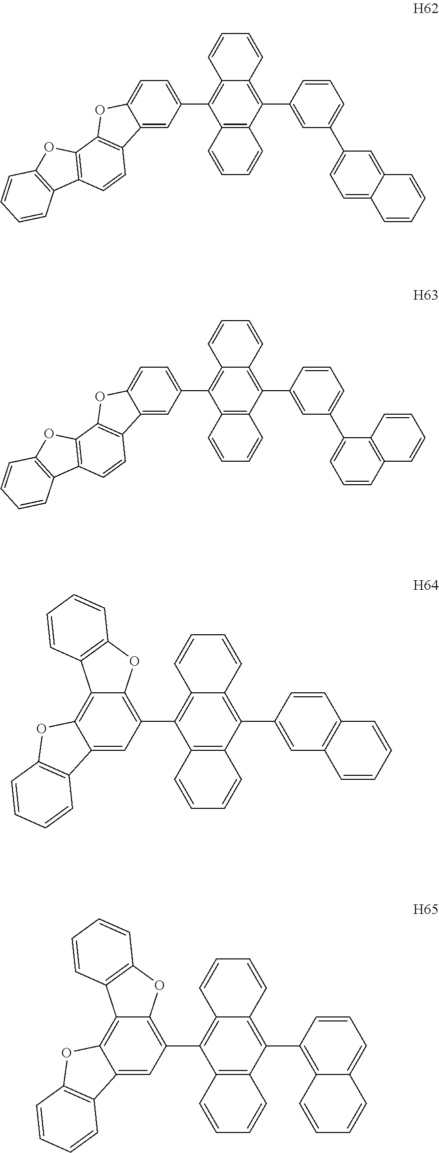 [SMILES: c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1]

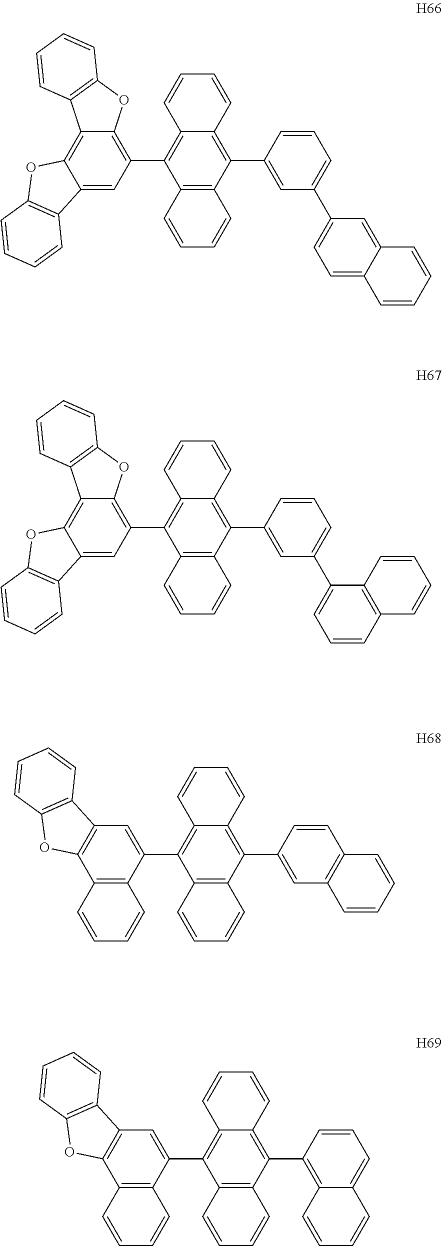 [SMILES: c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1]

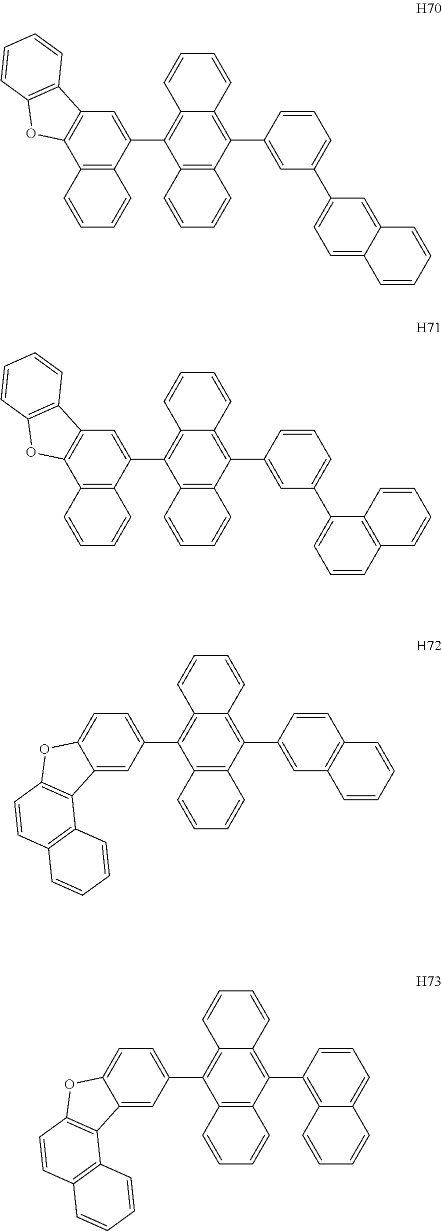 [SMILES: c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1 ; c1ccc2c(-c3c4ccccc4c(-c4ccc5oc6ccc7ccccc7c6c5c4)c4ccccc34)cccc2c1]

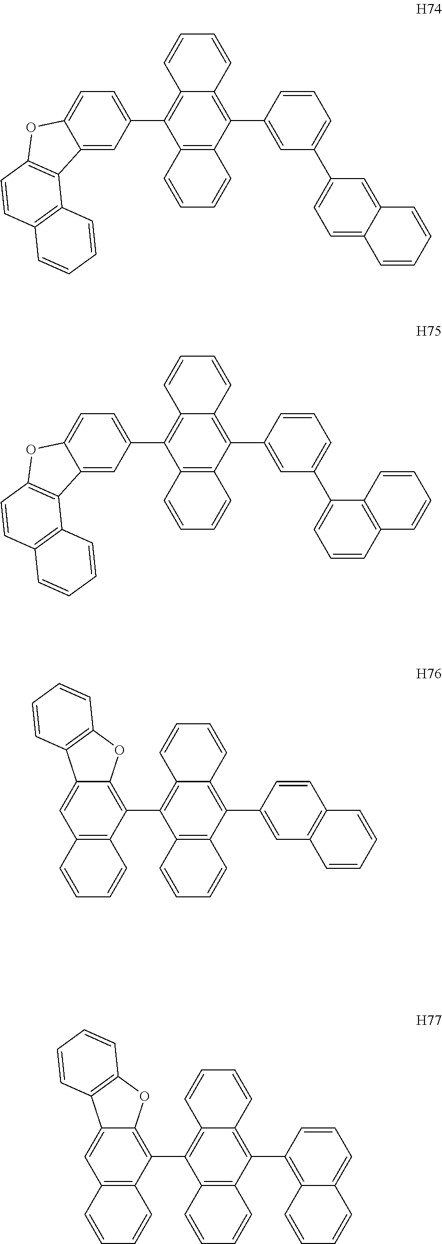 [SMILES: c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1 ; c1ccc2c(-c3c4ccccc4c(-c4c5ccccc5cc5c4oc4ccccc45)c4ccccc34)cccc2c1]

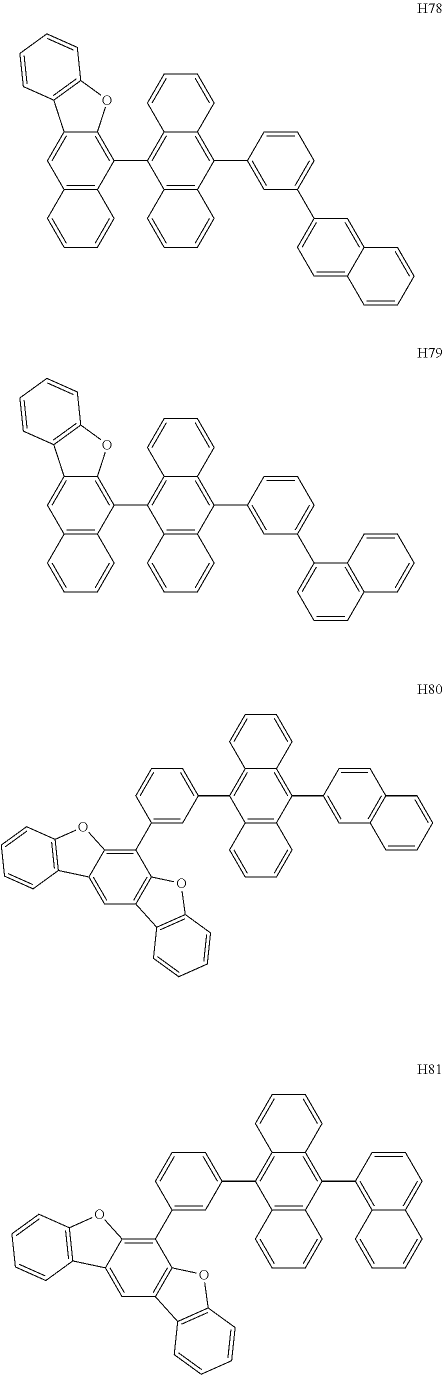 [SMILES: c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

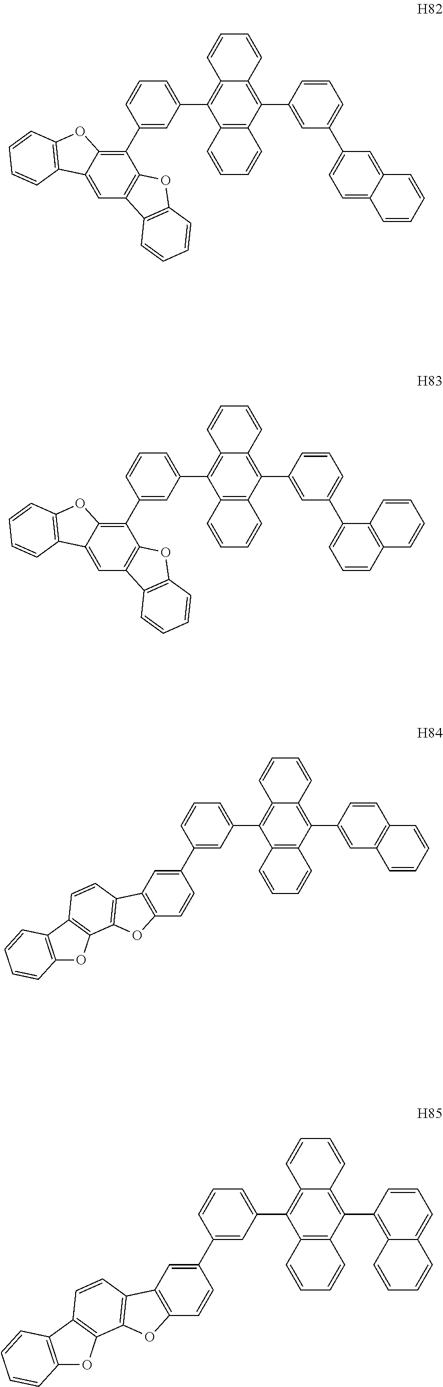 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1]

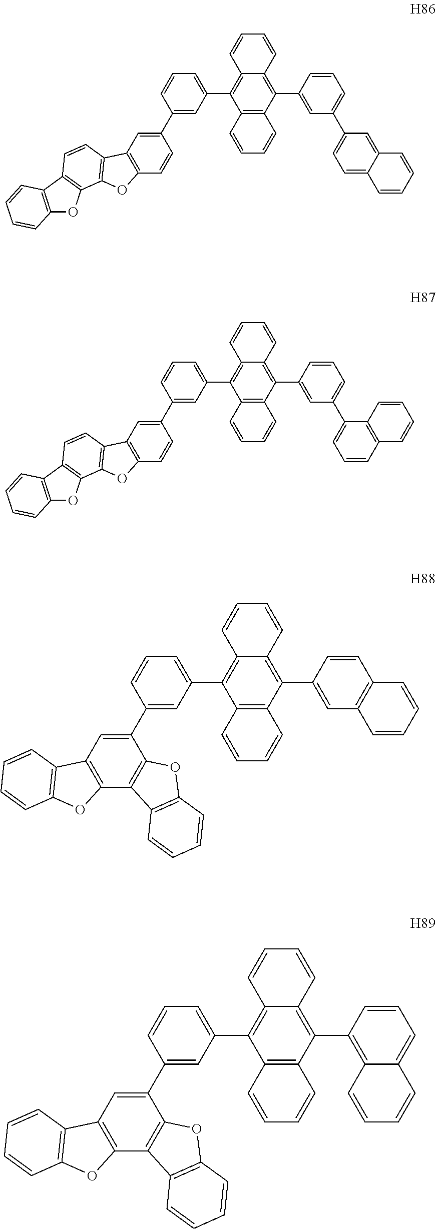 [SMILES: c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1]

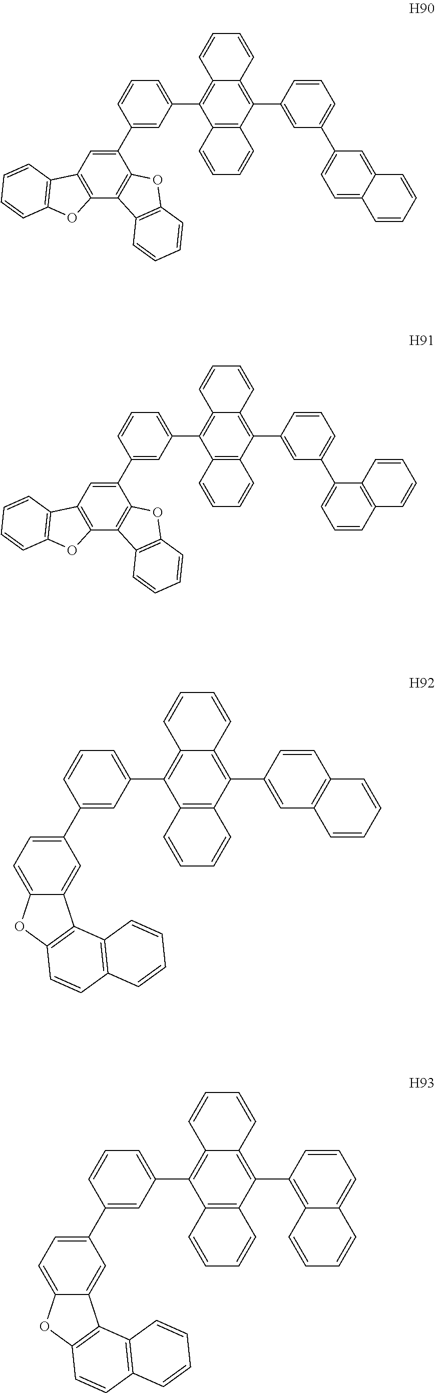 [SMILES: c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1 ; c1cc(-c2ccc3oc4ccc5ccccc5c4c3c2)cc(-c2c3ccccc3c(-c3cccc4ccccc34)c3ccccc23)c1]

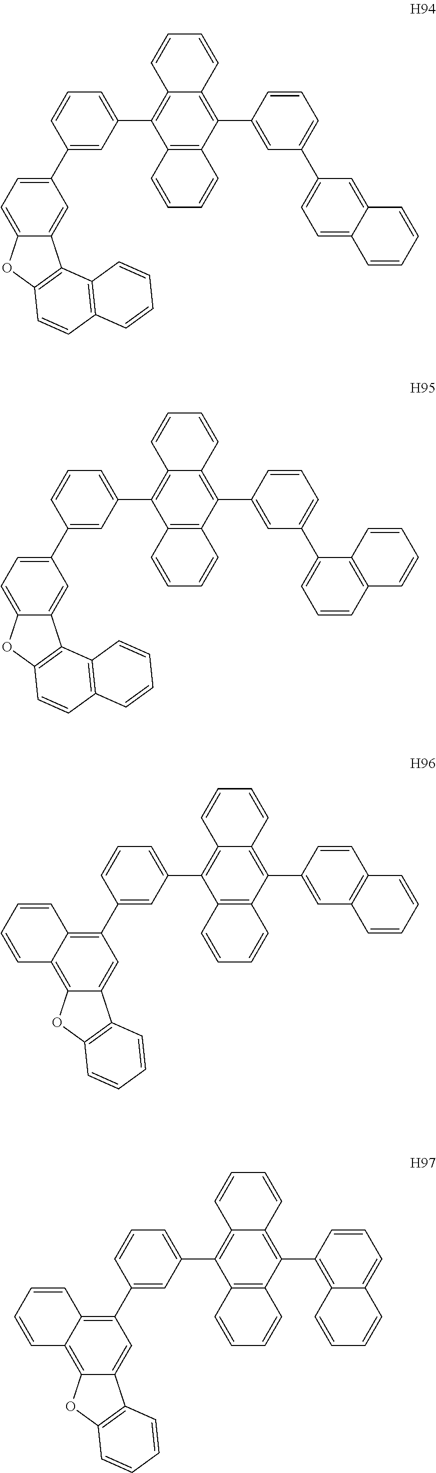 [SMILES: c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1]

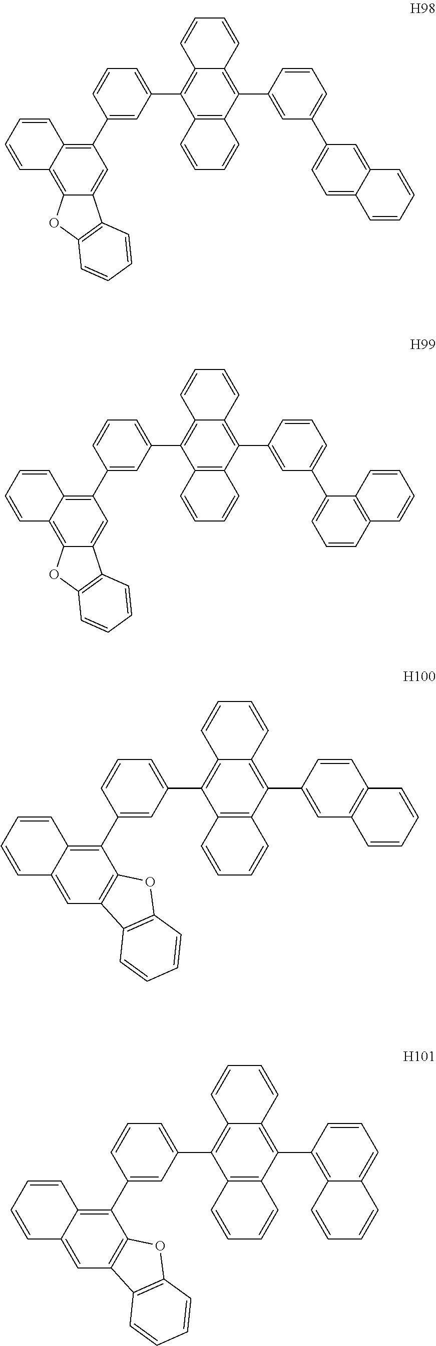 [SMILES: c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1]

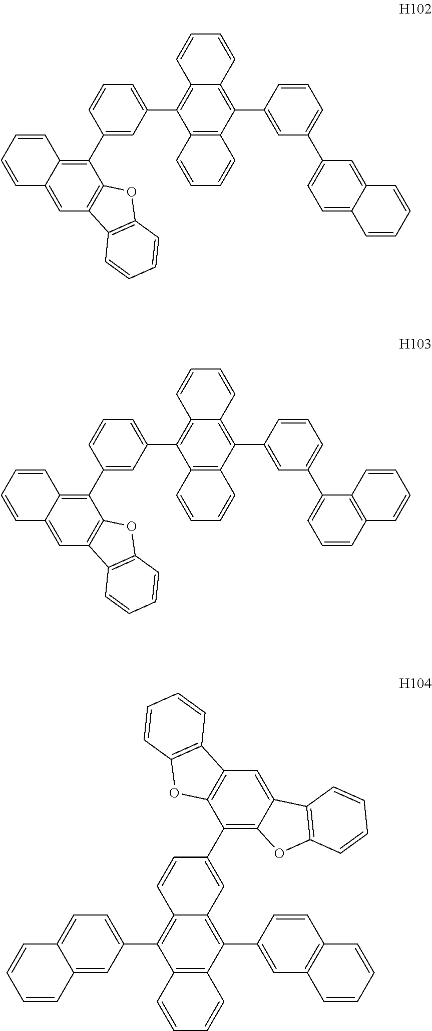 [SMILES: c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

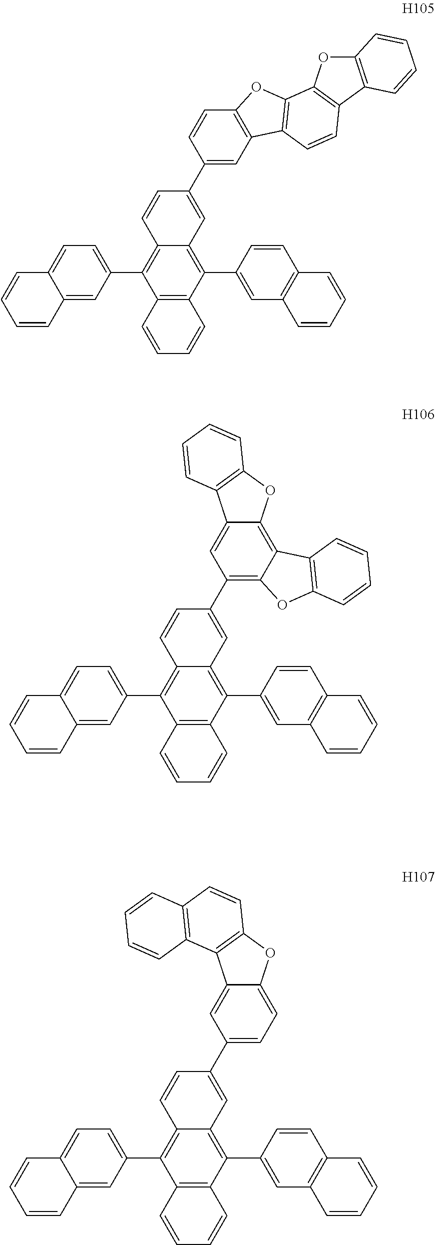 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

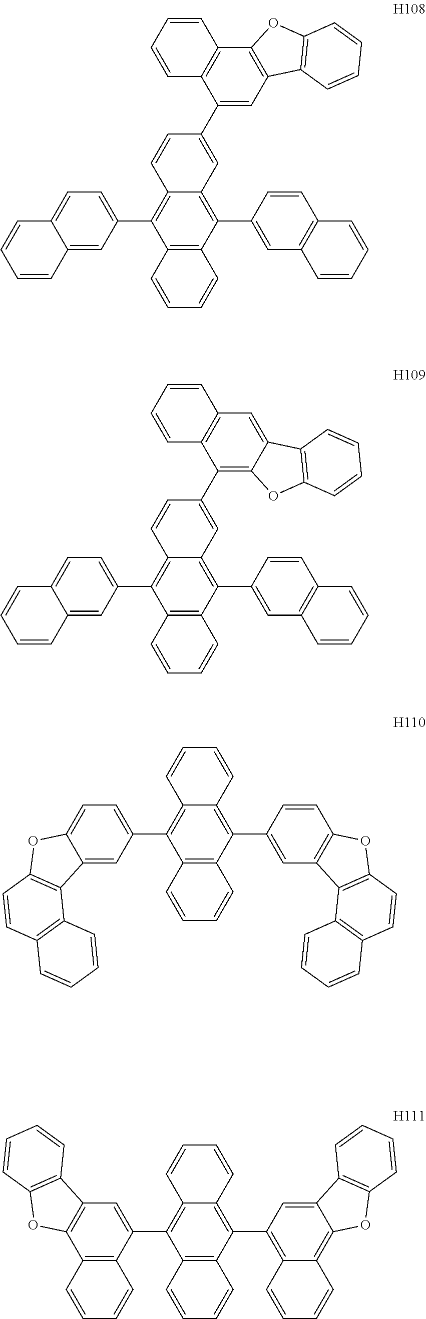 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21]

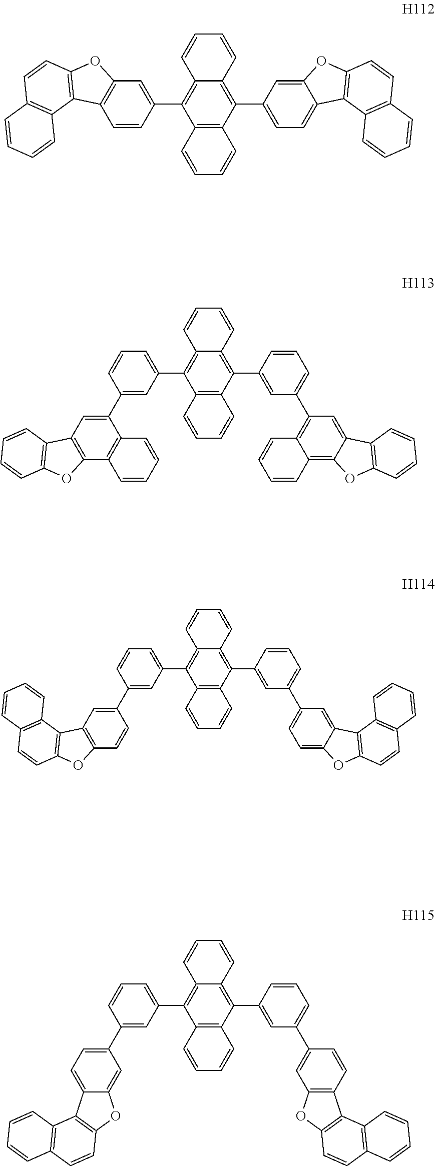 [SMILES: c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1]

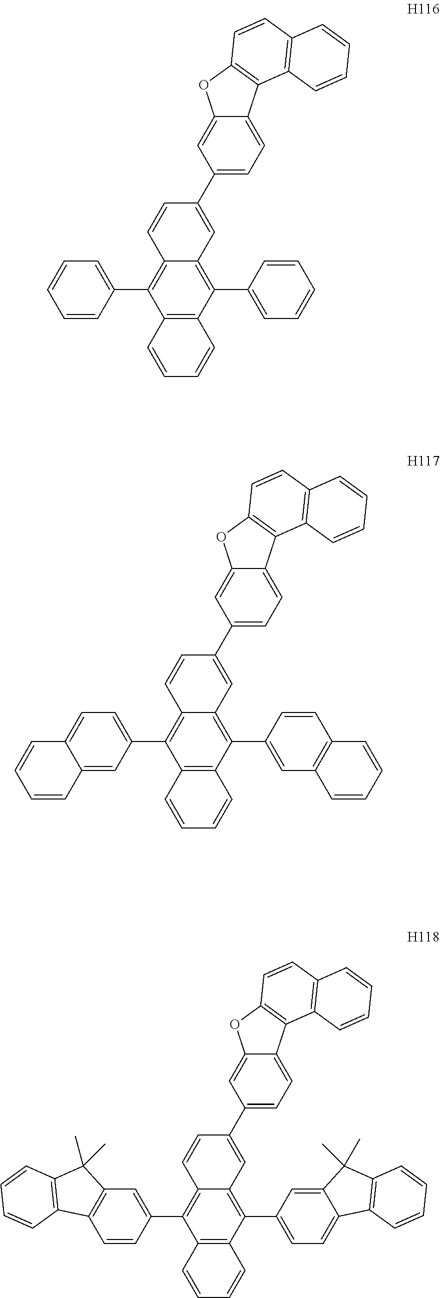 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

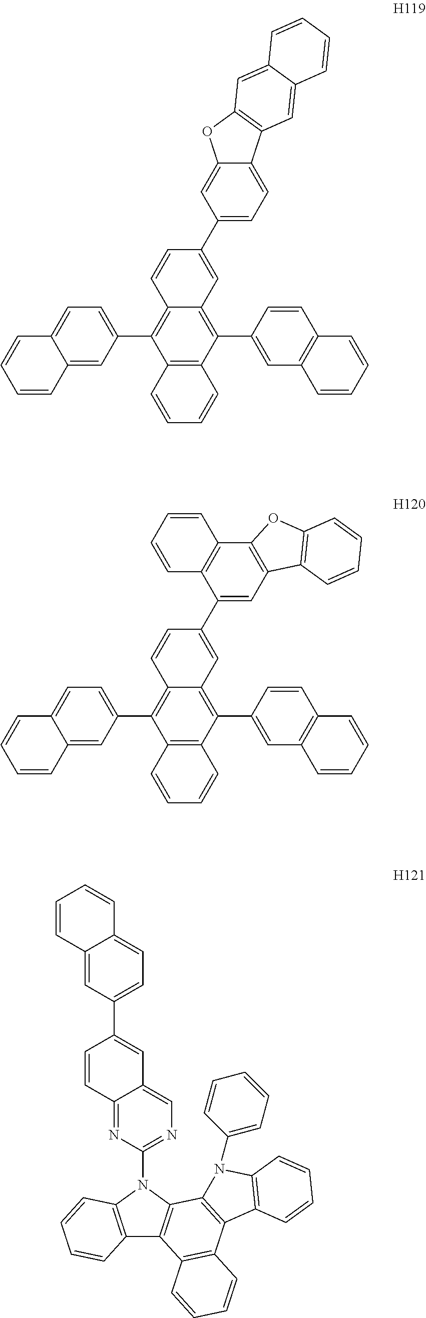 [SMILES: c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1]

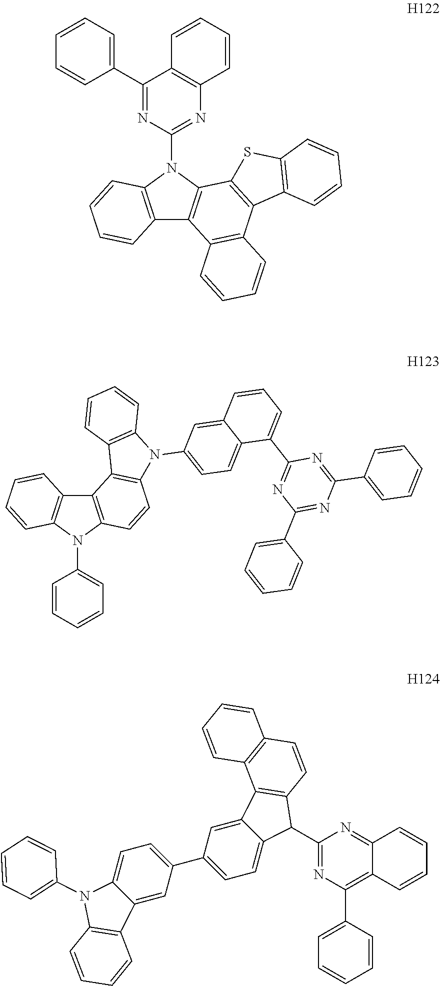 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(C3c4ccc(-c5ccc6c(c5)c5ccccc5n6-c5ccccc5)cc4-c4c3ccc3ccccc43)nc3ccccc23)cc1]

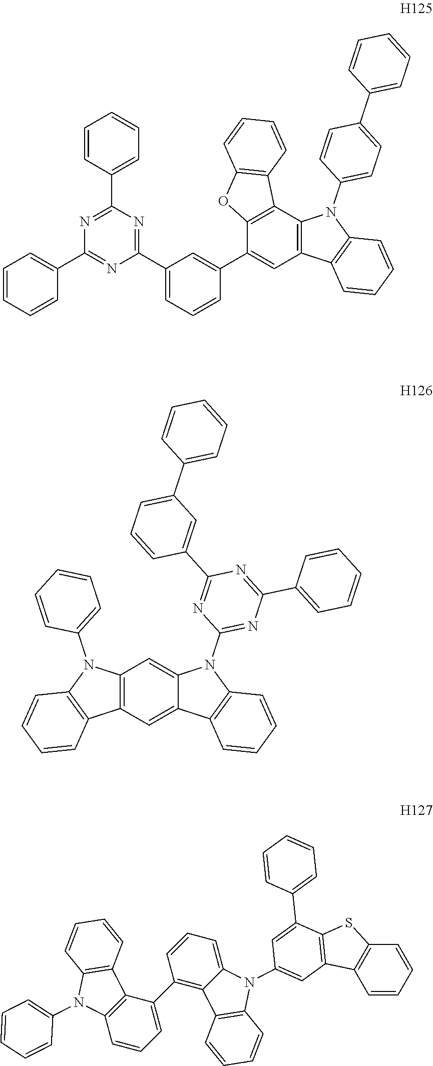 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1]

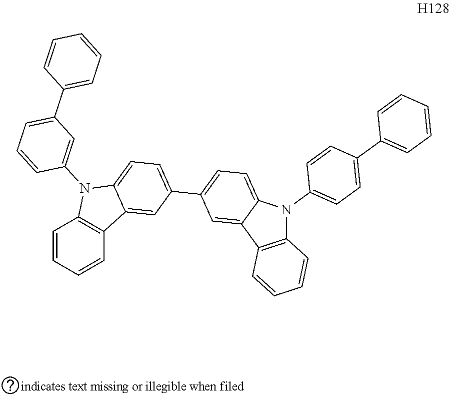 [SMILES: c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

In one or more embodiments, the phosphorescent dopant may include at least one transition metal as a center metal.

The phosphorescent dopant may include a monodentate ligand, a bidentate ligand, a tridentate ligand, a tetradentate ligand, a pentadentate ligand, a hexadentate ligand, or any combination thereof.

In some embodiments, the phosphorescent dopant may be electrically neutral.

In one or more embodiments, the phosphorescent dopant may include an organometallic compound represented by Formula 401:

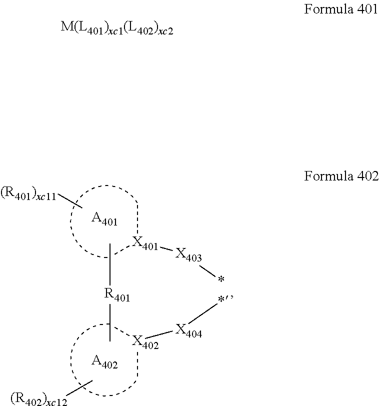

wherein, in Formulae 401 and 402,
M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, and when xc1 is 2 or greater, two or more of L₄₀₁(s) may be identical to or different from each other,
L₄₀₂ may be an organic ligand, and xc2 may be an integer from 0 to 4, and when xc2 is 2 or greater, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)*, *—C(Q₄₁₁)(Q₄₁₂)-*′, *—C(Q₄₁₁)═C(Q₄₁₂)-*′, *—C(Q₄₁₁)=*′, or *═C(Q₄₁₁*′,
X₄₀₃ and X₄₀₄ may each independently be a chemical bond (e.g., a covalent bond or a coordinate bond), O, S, N(Q₄₁₃), B(Q₄₁₃), P(Q₄₁₃), C(Q₄₁₃)(Q₄₁₄), or Si(Q₄₁₃)(Q₄₁₄),
wherein Q₄₁₁ to Q₄₁₄ may each be understood by referring to the description of Q₁ provided herein,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
wherein Q₄₀₁ to Q₄₀₃ may each be understood by referring to the description of Q₁ provided herein,
xc11 and xc12 may each independently be an integer from 0 to 10, and
* and *′ in Formula 402 each indicate a binding site to M in Formula 401.

For example, in some embodiments, in Formula 402, i) X₄₀₁ may be nitrogen, and X₄₀₂ may be carbon, or ii) X₄₀₁ and X₄₀₂ may each be nitrogen.

In one or more embodiments, when xc1 in Formula 402 is 2 or greater, two ring A₄₀₁ (s) of at least two L₄₀₁ (s) may optionally be bound via T₄₀₂ as a linking group, and/or two ring A₄₀₂(s) may optionally be bound via T₄₀₃ as a linking group (see Compounds PD1 to PD4 and PD7). T₄₀₂ and T₄₀₃ may each be understood by referring to the description of T₄₀₁ provided herein.

In Formula 401, L₄₀₂ may be any suitable organic ligand. For example, in some embodiments, L₄₀₂ may be a halogen, a diketone group (e.g., an acetylacetonate group), a carboxylic acid group (e.g., a picolinate group), —C(═O), an isonitrile group, —CN, a phosphorus-containing group (e.g., a phosphine group or a phosphite group), or any combination thereof.

In one or more embodiments, the phosphorescent dopant may be, for example, one selected from among Compounds PD1 to PD39, and/or any combination thereof:

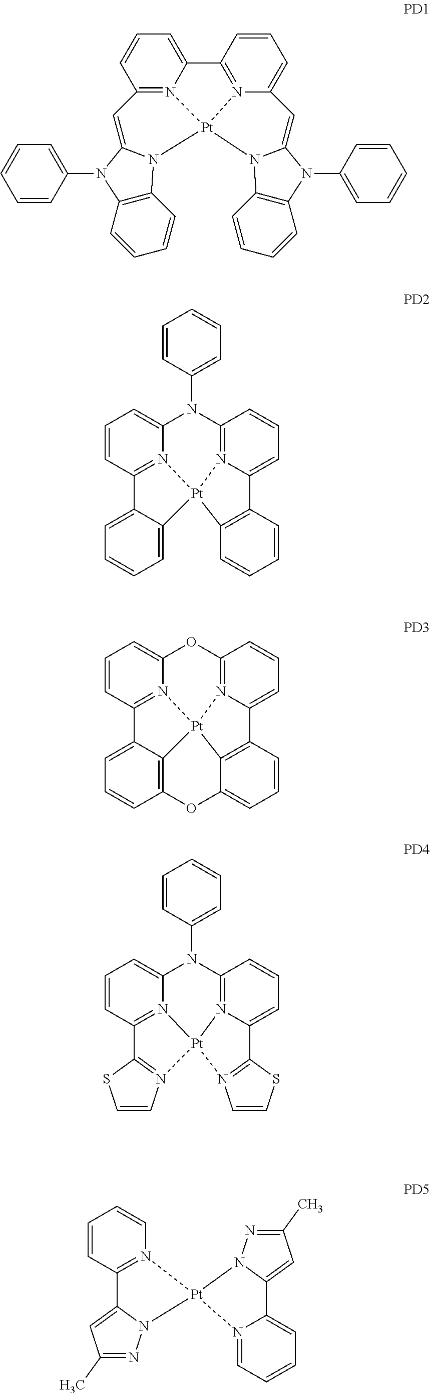

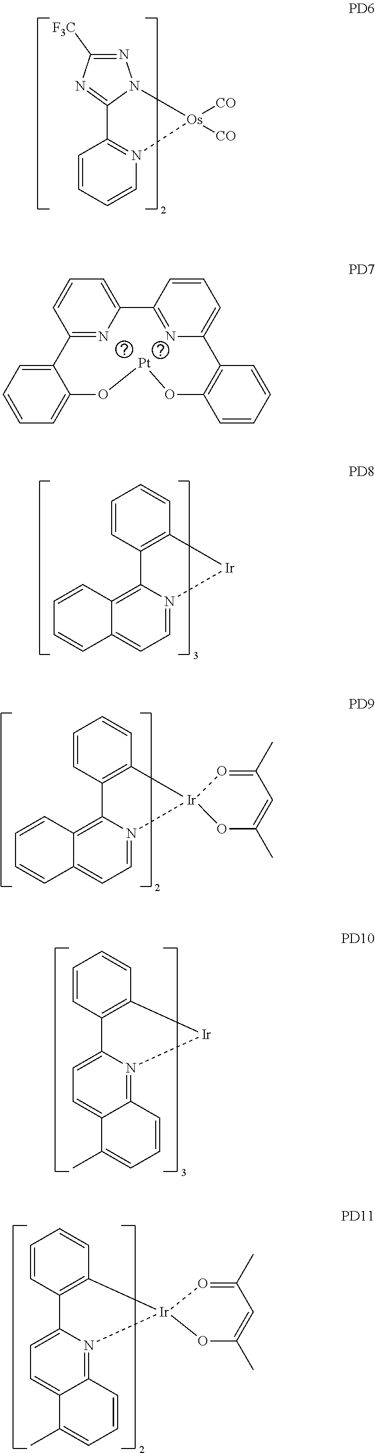

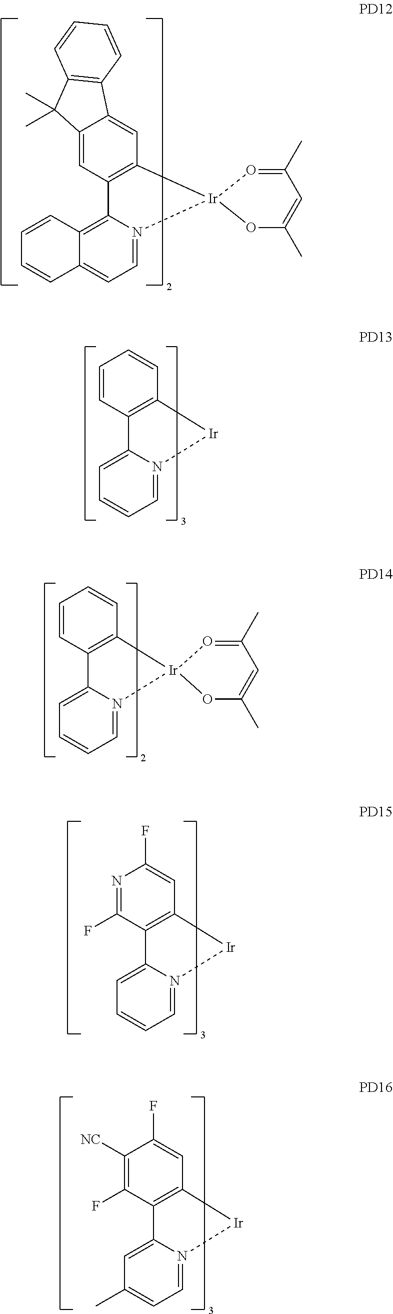

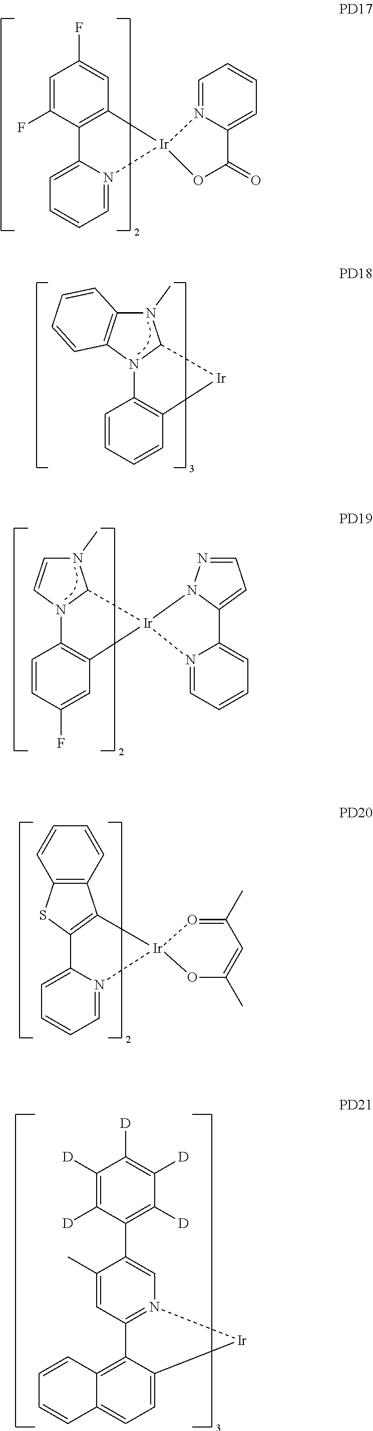

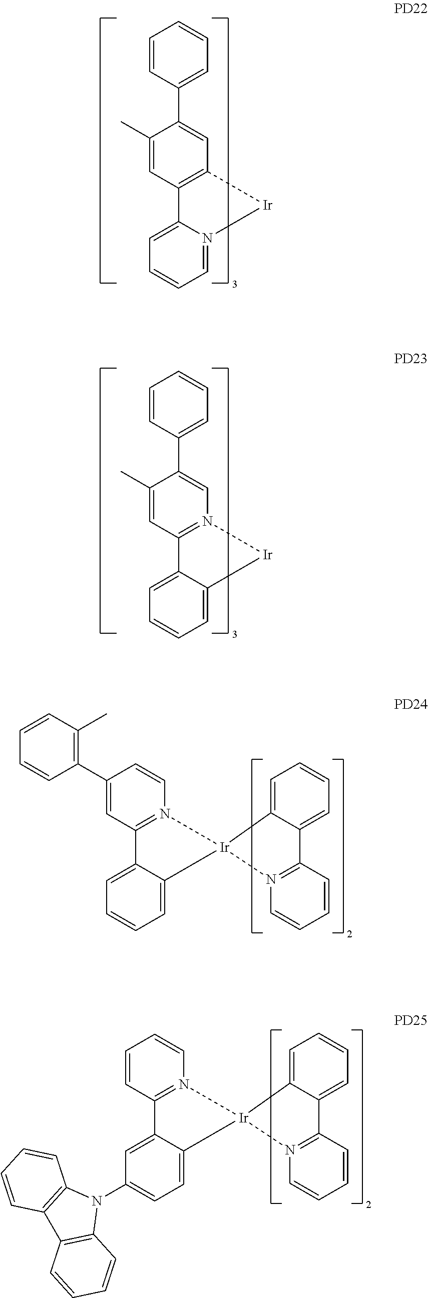

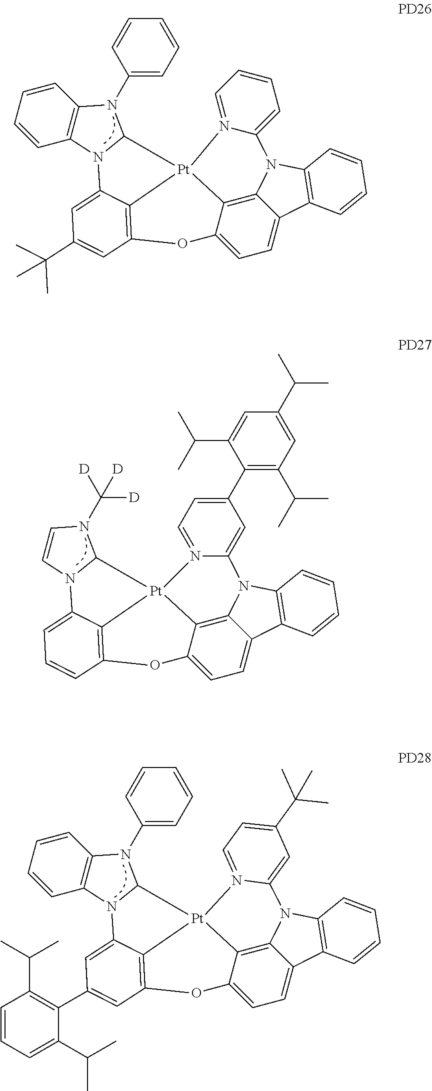

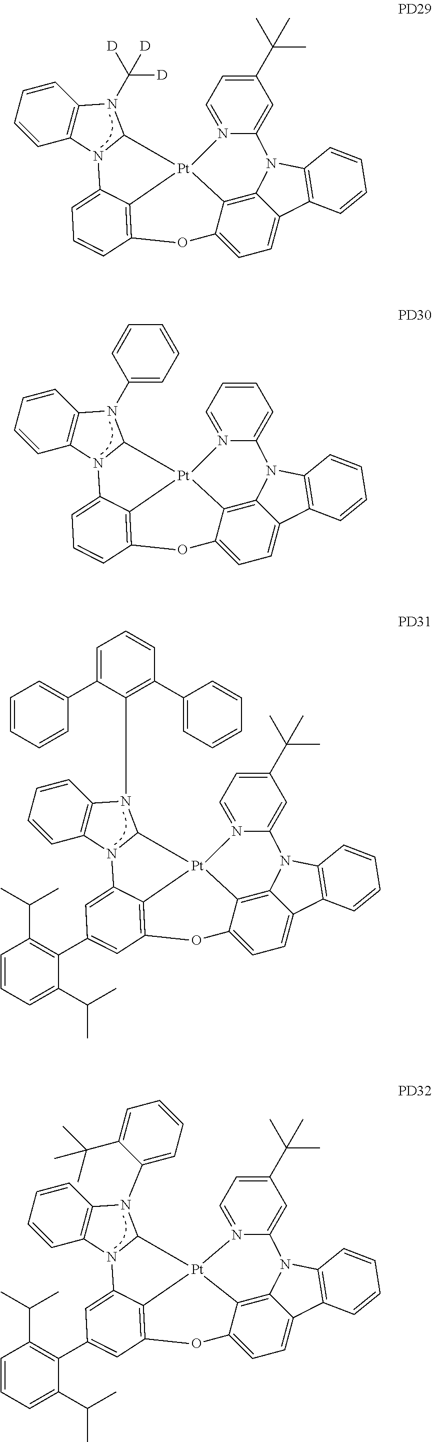

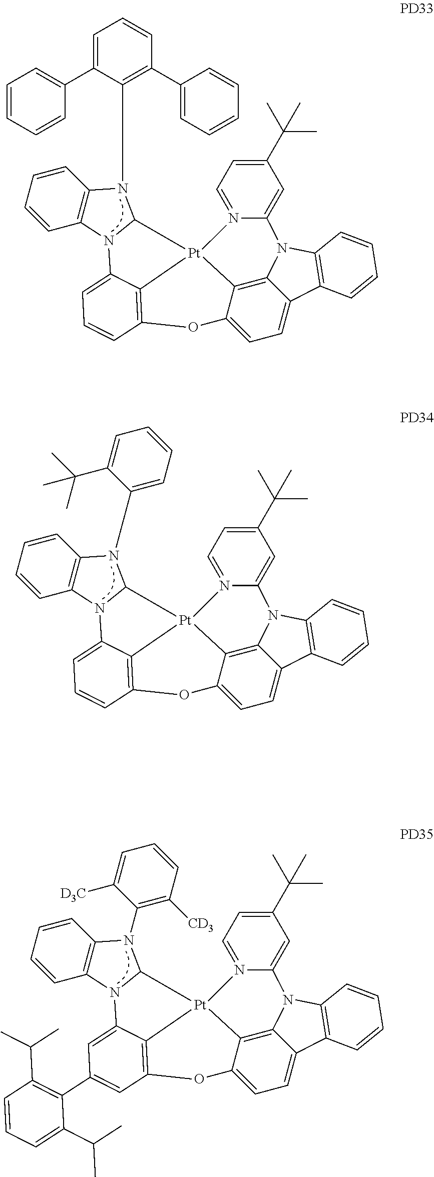

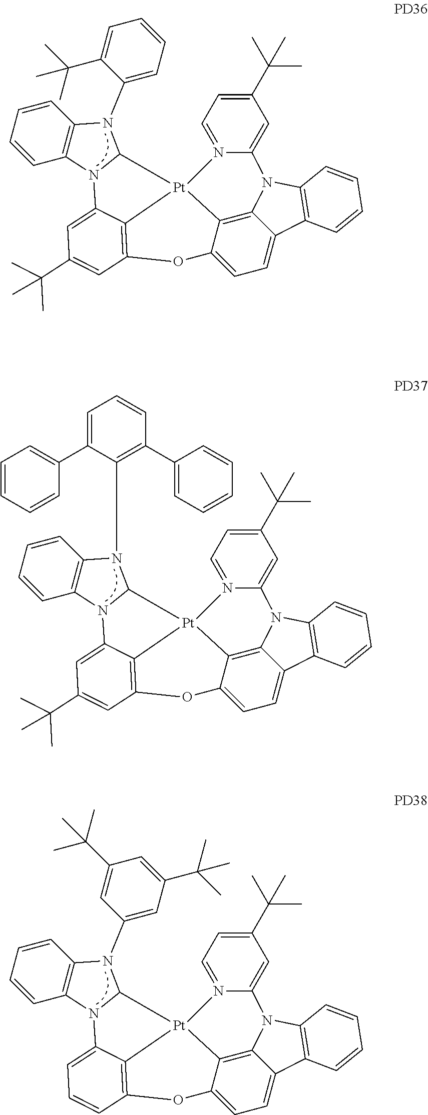

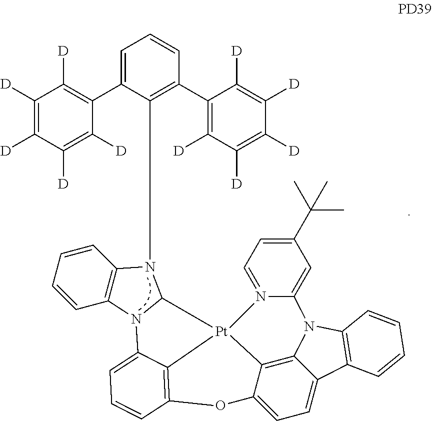

### Fluorescent Dopant

In one or more embodiments, the fluorescent dopant may include an amine-containing compound represented by Formula 501, a styryl-containing compound, or any combination thereof:

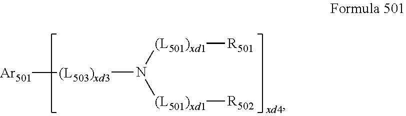

wherein, in Formula 501,
Ar₅₀₁, L₅₀₁ to L₅₀₅, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xd1 to xd3 may each independently be 0, 1, 2, or 3, and
xd4 may be 1, 2, 3, 4, 5, or 6.

In some embodiments, in Formula 501, Ar₅₀₁ may include a condensed ring group (e.g., an anthracene group, a chrysene group, or a pyrene group) in which at least three monocyclic groups are condensed.

In some embodiments, xd4 in Formula 501 may be 2.

In some embodiments, the fluorescent dopant may include one selected from among Compounds FD1 to FD37, 4,4′-bis(2,2-diphenylvinyl)-1,1′-biphenyl (DPVBi), 4,4′-bis[4-(N,N-diphenylamino)styryl]biphenyl (DPAVBi), and/or any combination thereof:

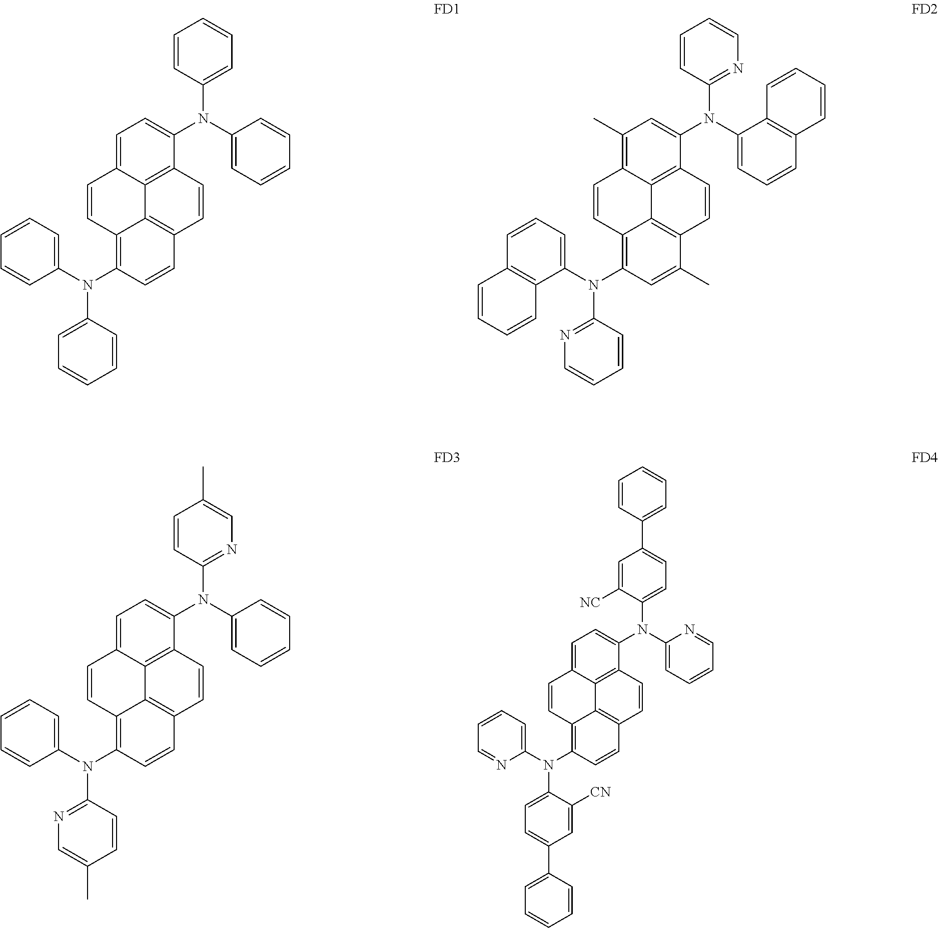 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1 ; N#Cc1cc(-c2ccccc2)ccc1N(c1ccccn1)c1ccc2ccc3c(N(c4ccccn4)c4ccc(-c5ccccc5)cc4C#N)ccc4ccc1c2c43]

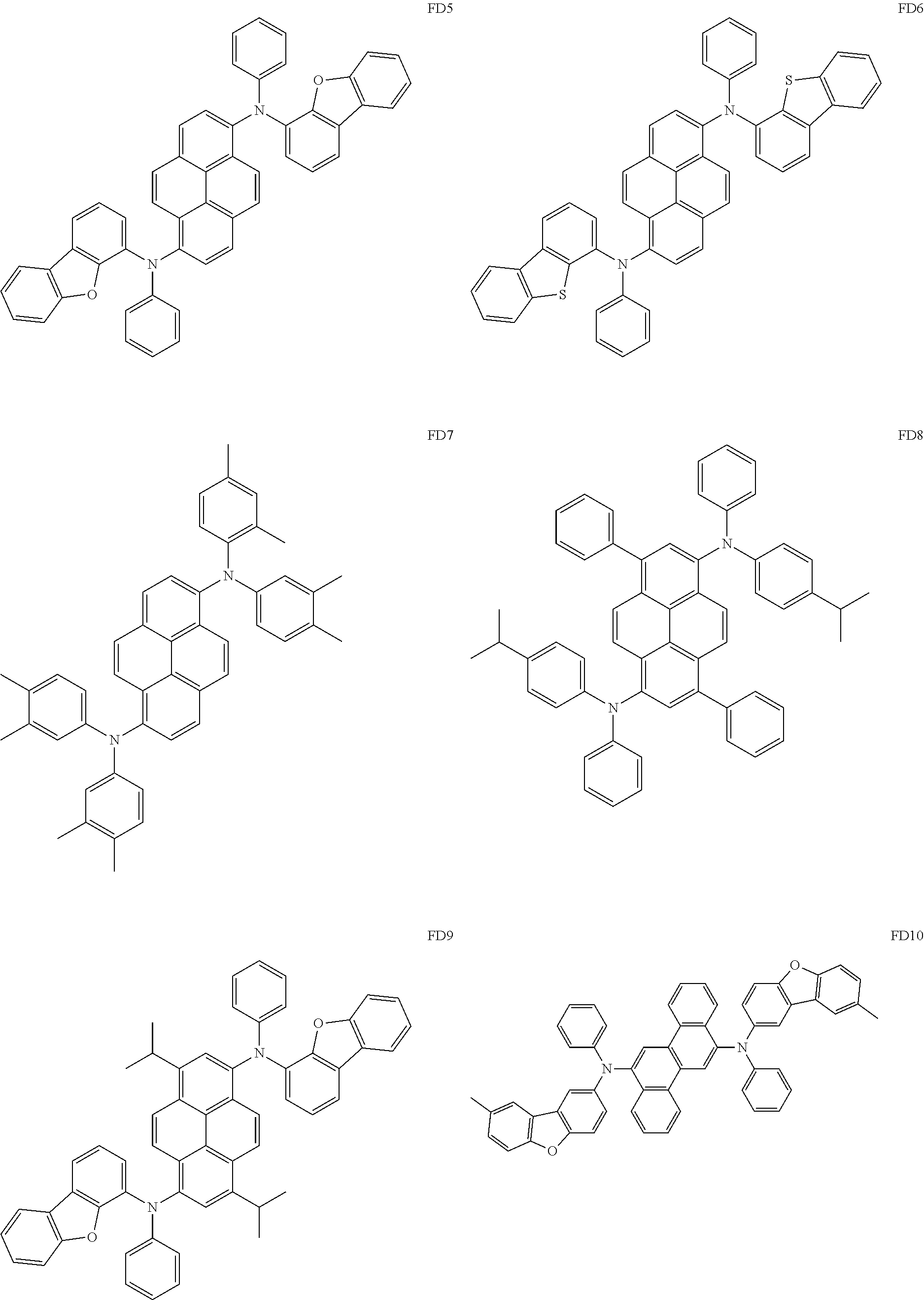

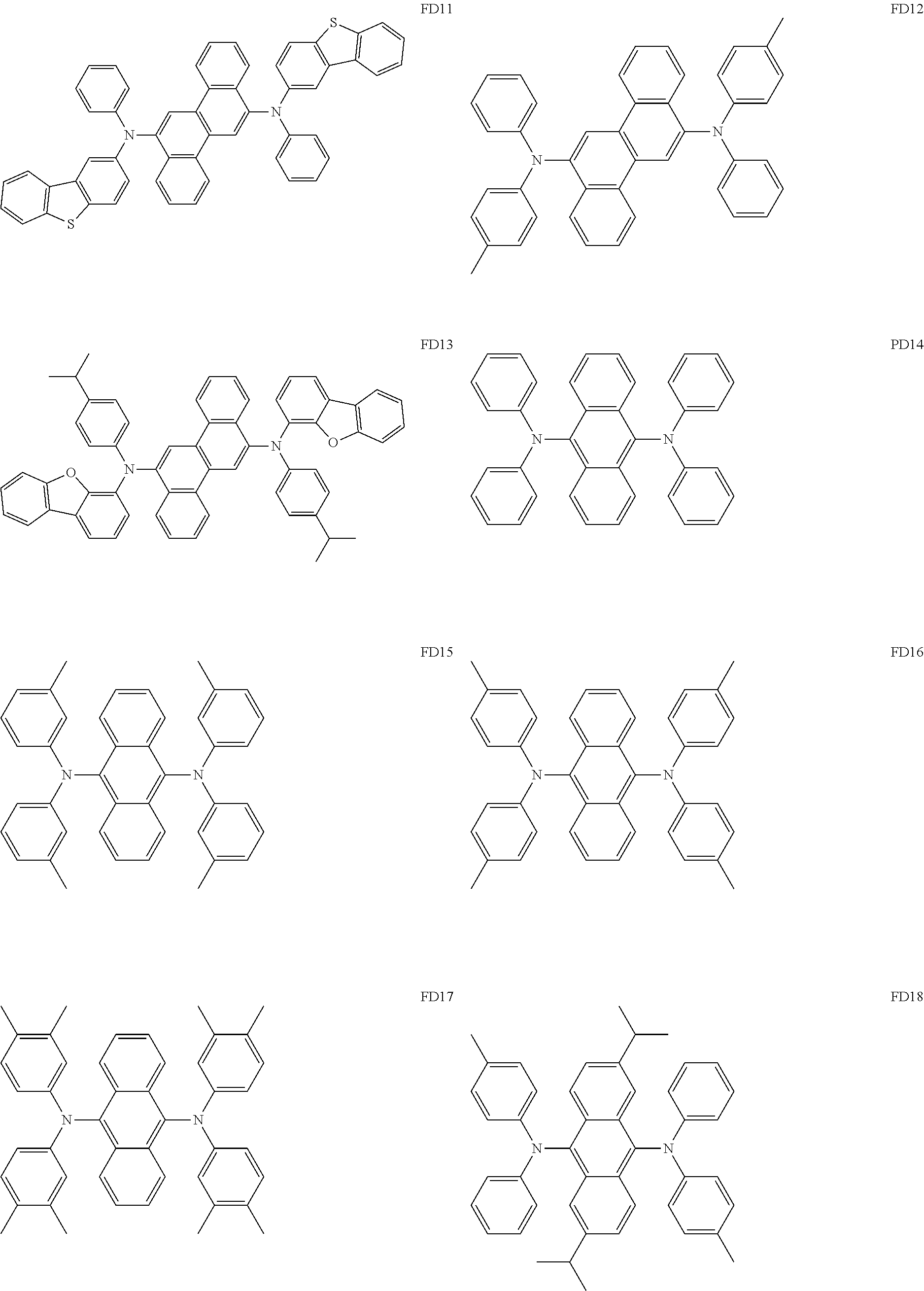

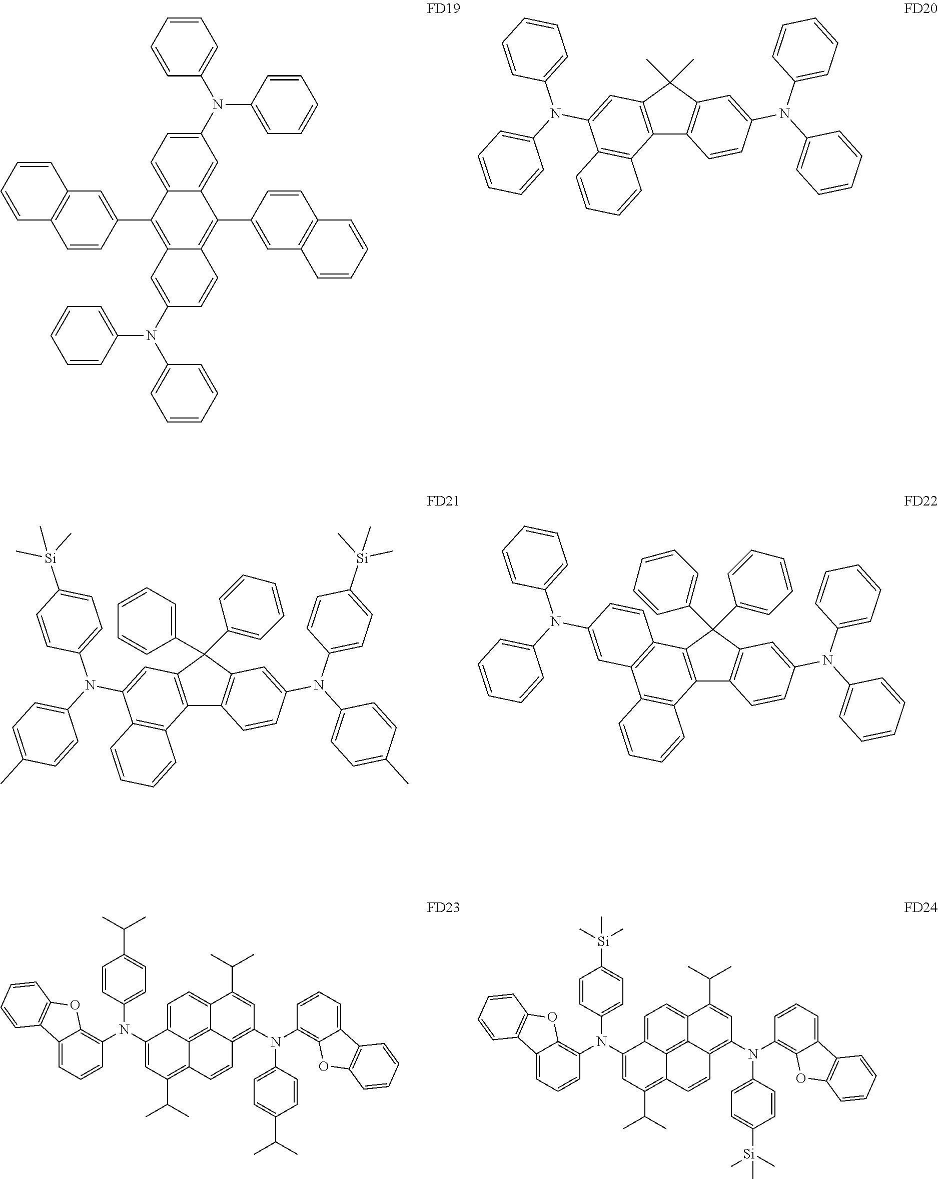

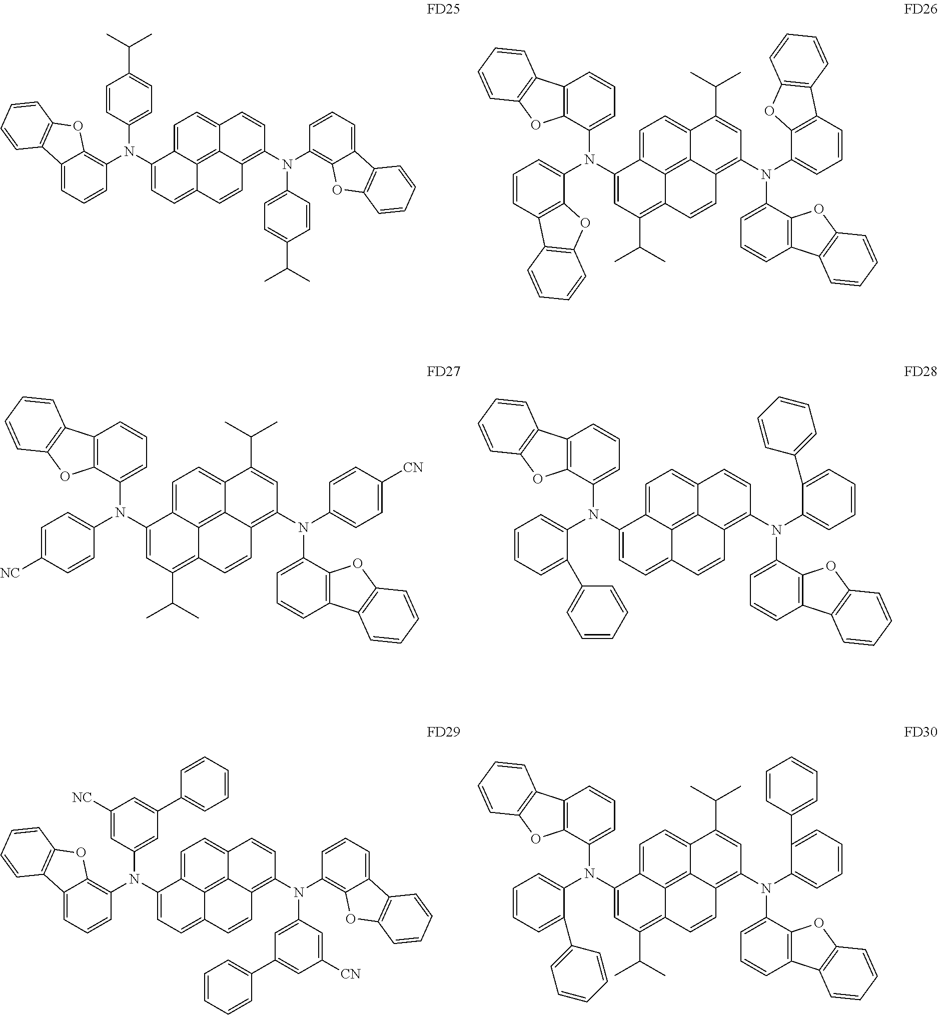

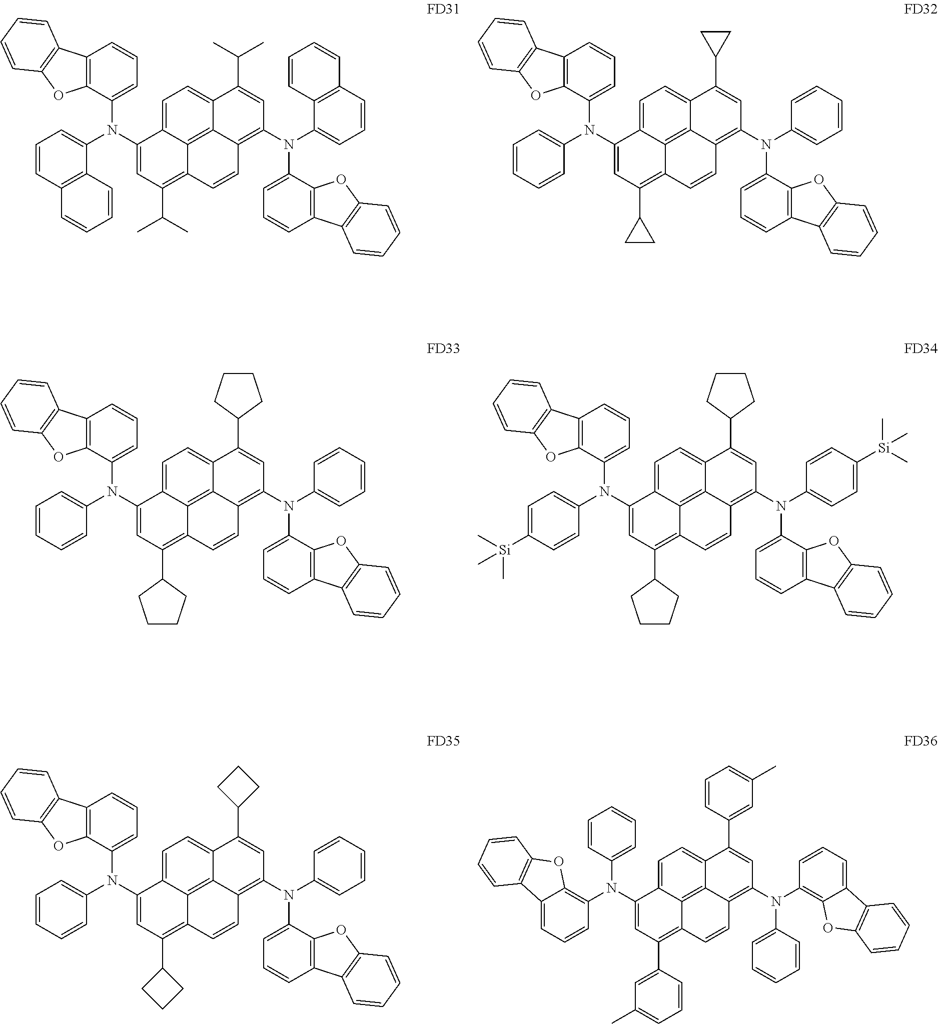

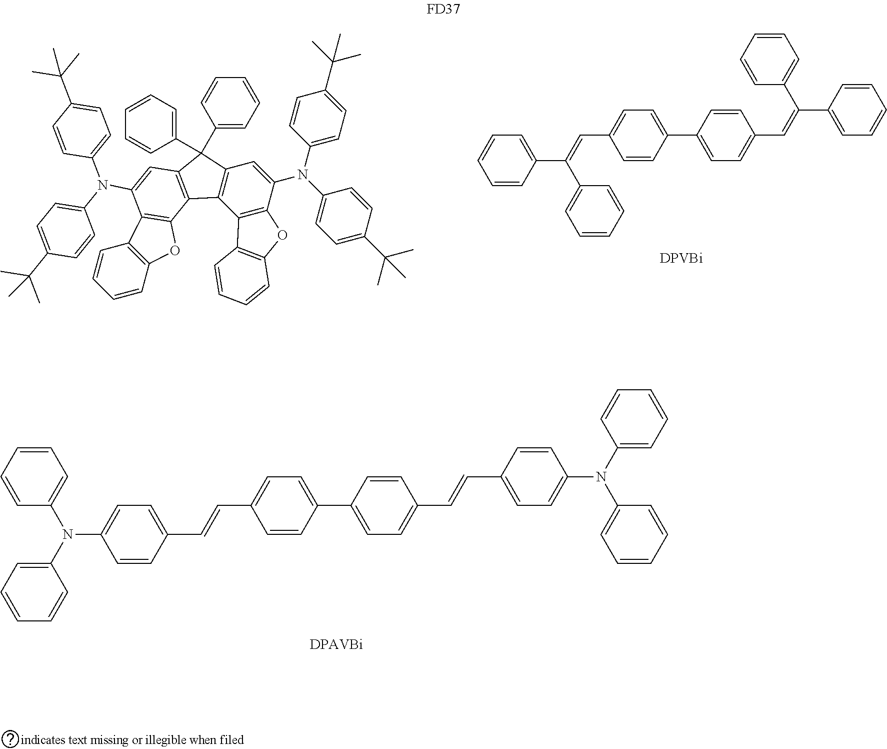

### Delayed Fluorescence Material

In one or more embodiments, the emission layer may include a delayed fluorescence material.

The delayed fluorescence material described herein may be any suitable compound that may be to emit delayed fluorescence according to a delayed fluorescence emission mechanism.

The delayed fluorescence material included in the emission layer may serve as a host or a dopant, depending on types (kinds) of other materials included in the emission layer.

In some embodiments, a difference between a triplet energy level (eV) of the delayed fluorescence material and a singlet energy level (eV) of the delayed fluorescence material may be about 0 eV or greater and about 0.5 eV or less. When the difference between the triplet energy level (eV) of the delayed fluorescence material and the singlet energy level (eV) of the delayed fluorescence material is within this range, up-conversion from the triplet state to the singlet state in the delayed fluorescence material may be effectively occurred, thus improving luminescence efficiency and/or the like of the light-emitting device 10.

In some embodiments, the delayed fluorescence material may include: i) a material including at least one electron donor (e.g., a rr electron-rich C₃-C₆₀ cyclic group such as a carbazole group and/or the like) and at least one electron acceptor (e.g., a sulfoxide group, a cyano group, a rr electron-deficient nitrogen-containing C₁-C₆₀ cyclic group, and/or the like), ii) a material including a C₈-C₆₀ polycyclic group including at least two cyclic groups condensed to each other and sharing boron (B), and/or iii) the like.

Non-limiting examples of the delayed fluorescence material may include at least one selected from Compounds DF1 to DF14:

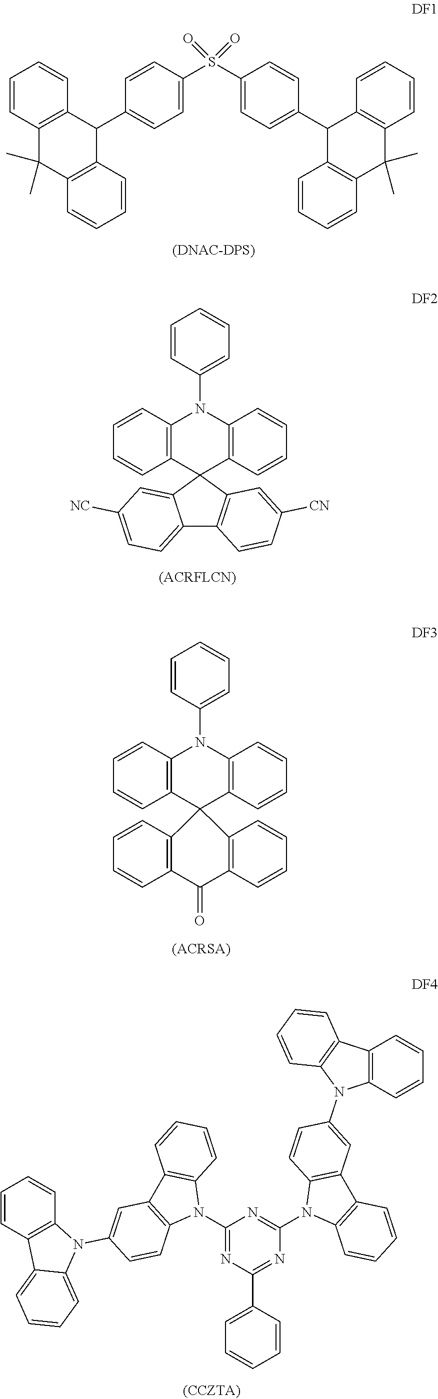

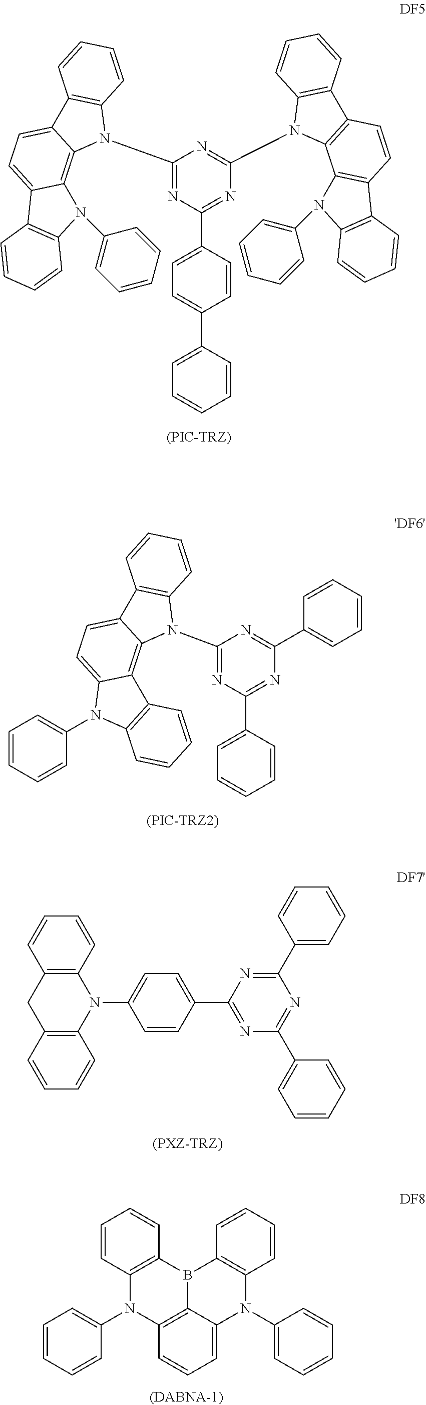

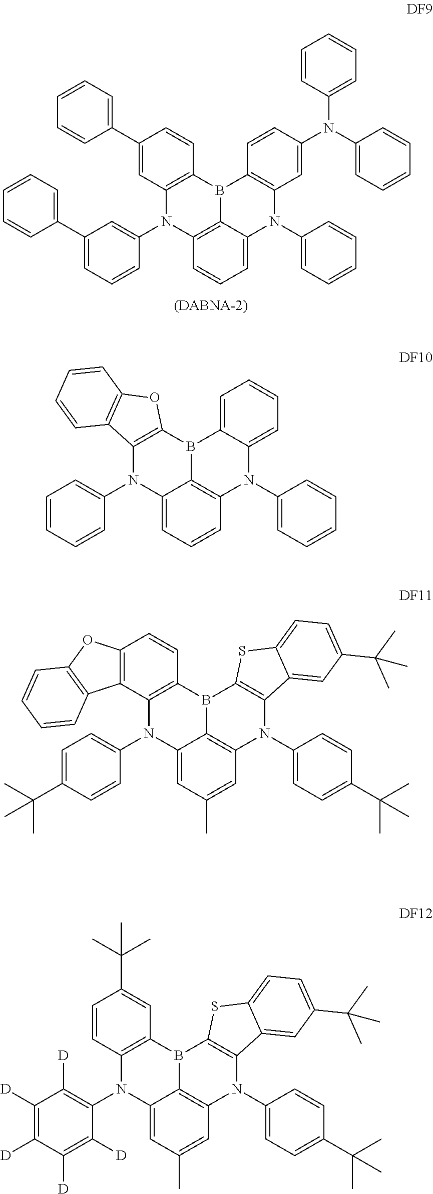

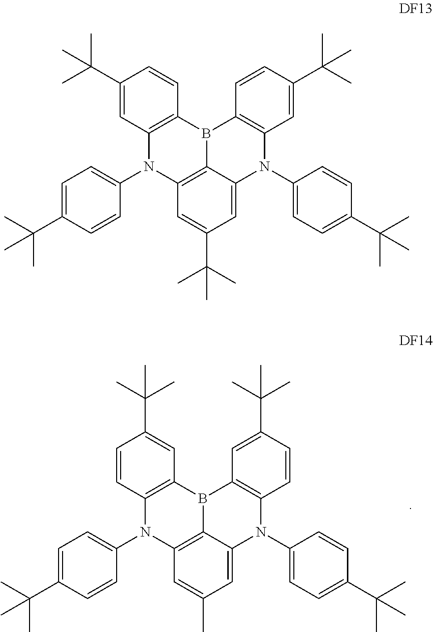

### Quantum Dots

In one or more embodiments, the emission layer may include quantum dots.

The term “quantum dot” as utilized herein refers to a crystal of a semiconductor compound and may include any suitable material capable of emitting emission wavelengths of one or more suitable lengths according to the size of the crystal.

The diameter of the quantum dot may be, for example, in a range of about 1 nm to about 10 nm.

Quantum dots may be synthesized by a wet chemical process, an organic metal chemical vapor deposition process, a molecular beam epitaxy process, or any similar process.

The wet chemical process is a method of growing a quantum dot particle crystal by mixing a precursor material with an organic solvent. When the crystal grows, the organic solvent may naturally serve as a dispersant coordinated on the surface of the quantum dot crystal and control the growth of the crystal. Thus, the wet chemical method may be easier to perform than the vapor deposition process such a metal organic chemical vapor deposition (MOCVD) or a molecular beam epitaxy (MBE) process. Further, the growth of quantum dot particles may be controlled or selected with a lower manufacturing cost.

The quantum dot may include a group II-VI semiconductor compound; a group III-V semiconductor compound; a group III-VI semiconductor compound; a group I-III-VI semiconductor compound; a group IV-VI semiconductor compound; a group IV element or compound; or any combination thereof.

Non-limiting examples of the group II-VI semiconductor compound may include a binary compound such as CdS, CdSe, CdTe, ZnS, ZnSe, ZnTe, ZnO, HgS, HgSe, HgTe, MgSe, and/or MgS; a ternary compound such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound such as CdZnSeS, CdZnSeTe, CdZnSTe, CdHgSeS, CdHgSeTe, CdHgSTe, HgZnSeS, HgZnSeTe, and/or HgZnSTe; or a combination thereof.

Non-limiting examples of the group III-V semiconductor compound may include a binary compound such as GaN, GaP, GaAs, GaSb, AlN, AlP, AlAs, AlSb, InN, InP, InAs, and/or InSb; a ternary compound such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound such as GaAlNP, GaAlNAs, GaAlNSb, GaAlPAs, GaAlPSb, GalnNP, GaInNAs, GalnNSb, GaInPAs, GalnPSb, InAINP, InAINAs, InAINSb, InAIPAs, and/or InAIPSb; or any combination thereof. In some embodiments, the group Ill-V semiconductor compound may further include a group II element. Non-limiting examples of the group III-V semiconductor compound further including the group II element may include InZnP, InGaZnP, InAIZnP, and/or the like.

Non-limiting examples of the III-VI group semiconductor compound may include a binary compound such as GaS, GaSe, Ga₂Se₃, GaTe, InS, InSe, In₂S₃, In₂Se₃, InTe, and/or the like; a ternary compound such as InGaS₃, InGaSe₃, and/or the like; or a combination thereof.

Non-limiting examples of the group I—III-VI semiconductor compound may include a ternary compound such as AgInS, AgInS₂, CuInS, CulnS₂, CuGaO₂, AgGaO₂, or AgAlO₂; or a combination thereof.

Non-limiting examples of the group IV-VI semiconductor compound may include: a binary compound such as SnS, SnSe, SnTe, PbS, PbSe, and/or PbTe; a ternary compound such as SnSeS, SnSeTe, SnSTe, PbSeS, PbSeTe, PbSTe, SnPbS, SnPbSe, and/or SnPbTe; a quaternary compound such as SnPbSSe, SnPbSeTe, and/or SnPbSTe; or a combination thereof.

The group IV element or compound may be a single element material such as Si and/or Ge; a binary compound such as SiC and/or SiGe; or a combination thereof.

Individual elements included in a multi-element compound, such as a binary compound, a ternary compound, and a quaternary compound, may be present in a particle thereof at a substantially uniform or non-substantially uniform concentration.

The quantum dot may have a single structure in which the concentration of each element included in the quantum dot is substantially uniform or a core-shell double structure. In some embodiments, materials included in the core may be different from materials included in the shell.

The shell of the quantum dot may serve as a protective layer for preventing or reducing chemical denaturation of the core to maintain semiconductor characteristics and/or as a charging layer for imparting electrophoretic characteristics to the quantum dot. The shell may be a monolayer or a multilayer. An interface between the core and the shell may have a concentration gradient where a concentration of elements present in the shell decreases toward the core.

Non-limiting examples of the shell of the quantum dot may include metal, metalloid, or nonmetal oxide, a semiconductor compound, or a combination thereof. Non-limiting examples of the metal, the metalloid, or the nonmetal oxide suitable as a shell may include a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄, and/or NiO; a ternary compound such as MgAl₂O₄, CoFe₂O₄, NiFe₂O₄, and/or CoMn₂O₄; or any combination thereof. Non-limiting examples of the semiconductor compound suitable as a shell may include: a group II-VI semiconductor compound; a group III-V semiconductor compound; a group III-VI semiconductor compound; a group 1-III-VI semiconductor compound; a group IV-VI semiconductor compound; or any combination thereof. In some embodiments, the semiconductor compound suitable as a shell may be CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

The quantum dot may have a full width of half maximum (FWHM) of an emission spectrum of about 45 nm or less, about 40 nm or less, or about 30 nm or less. When the FWHM of the quantum dot is within this range, color purity or color reproducibility may be improved. In some embodiments, because light emitted through the quantum dots is emitted in all directions, an optical viewing angle may be improved.

In some embodiments, the quantum dot may be a substantially spherical, pyramidal, multi-arm, or cubic nanoparticle, nanotube, nanowire, nanofiber, or nanoplate particle.

By adjusting the size of the quantum dot, the energy band gap of the quantum dot may also be adjusted, thereby obtaining light of one or more suitable wavelengths in a quantum dot emission layer. By utilizing quantum dots of one or more suitable sizes, a light-emitting device that may be to emit light of one or more suitable wavelengths may be realized. In some embodiments, the size of the quantum dot may be selected such that the quantum dot may be to emit red, green, and/or blue light. In some embodiments, the size of the quantum dot may be selected such that the quantum dot may be to emit white light by combining one or more suitable light colors.

### Electron Transport Region in Interlayer 130

The electron transport region may have i) a single-layered structure including (e.g., consisting of) a single layer including (e.g., consisting of) a single material, ii) a single-layered structure including (e.g., consisting of) a single layer including a plurality of different materials, or iii) a multi-layered structure having a plurality of layers including a plurality of different materials.

The electron transport region may include a buffer layer, a hole blocking layer, an electron control layer, an electron transport layer, an electron injection layer, or any combination thereof.

In some embodiments, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein constituent layers of each structure are sequentially stacked on the emission layer in each stated order.

In one or more embodiments, the electron transport region (e.g., a buffer layer, a hole blocking layer, an electron control layer, or an electron transport layer in the electron transport region) may include a metal-free compound including at least one rr electron-deficient nitrogen-containing C₁-C₆₀ cyclic group.

In some embodiments, the electron transport region may include a compound represented by Formula 601:

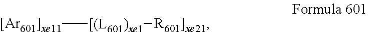

wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R10a, a C₁-C₆₀ heterocyclic group unsubstituted or substituted with at least one R10a, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
wherein Q₆₀₁ to Q₆₀₃ may each be understood by referring to the description of Q₁ provided herein,
xe21 may be 1, 2, 3, 4, or 5, and
at least one selected from among Ar₆₀₁, L₆₀₁, and R₆₀₁ may each independently be a rr electron-deficient nitrogen-containing C₁-C₆₀ cyclic group unsubstituted or substituted with at least one R₁₀ₐ.

For example, in some embodiments, in Formula 601, when xe11 is 2 or greater, two or more of Ar₆₀₁(s) may be bound to each other via a single bond.

In some embodiments, in Formula 601, Ar₆₀₁ may be a substituted or unsubstituted anthracene group.

In some embodiments, the electron transport region may include a compound represented by Formula 601-1:

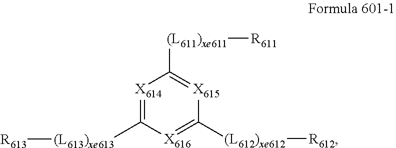

wherein, in Formula 601-1,
X₆₁₄ may be N or C(R₆₁₄), X₆₁₅ may be N or C(R₆₁₅), X₆₁₆ may be N or C(R₆₁₆), and at least one selected from among X₆₁₄ to X₆₁₆ may be N,
L₆₁₁ to L₆₁₃ may each be understood by referring to the description of L₆₀₁ provided herein,
xe611 to xe613 may each be understood by referring to the description of xe1 provided herein,
R₆₁₁ to R₆₁₃ may each be understood by referring to the description of R₆₀₁ provided herein, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, in Formulae 601 and 601-1, xe1 and xe611 to xe613 may each independently be 0, 1, or 2.

In one or more embodiments, the electron transport region may include at least one selected from among Compounds ET1 to ET45, 2,9-dimethyl-4,7-diphenyl-1,10-phenanthroline (BCP), 4,7-diphenyl-1,10-phenanthroline (Bphen), tris(8-hydroxyquinolinato)aluminum (Alq3), bis(2-methyl-8-quinolinolato-N1,O8)-(1,1′-biphenyl-4-olato)aluminum (BAlq), 3-(4-biphenyl)-4-phenyl-5-tert-butylphenyl-1,2,4-triazole (TAZ), 4-(naphthalen-1-yl)-3,5-diphenyl-4H-1,2,4-triazole (NTAZ), and/or any combination thereof:

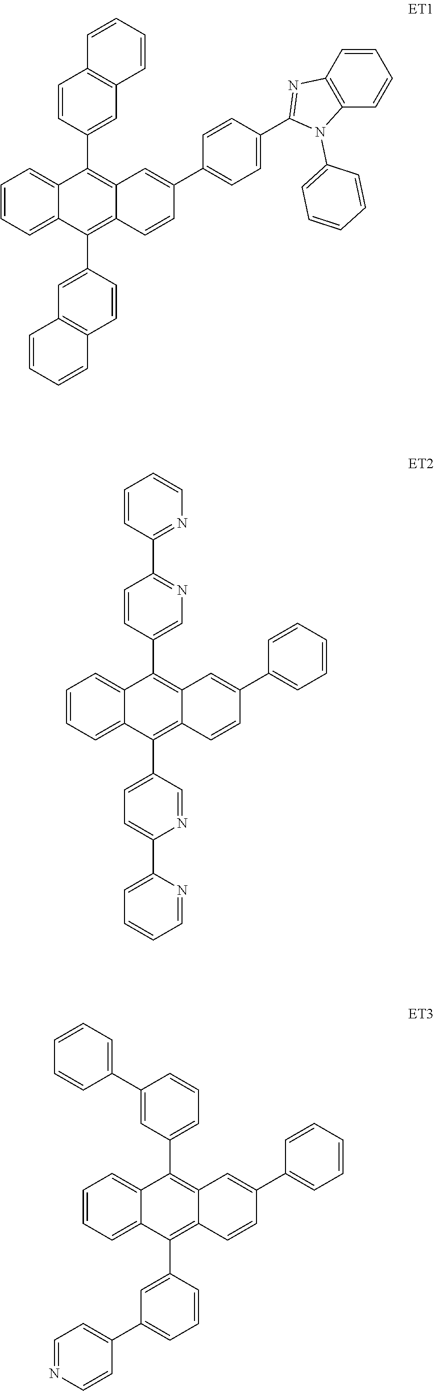 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2cccc(-c3c4ccccc4c(-c4cccc(-c5ccncc5)c4)c4ccc(-c5ccccc5)cc34)c2)cc1]

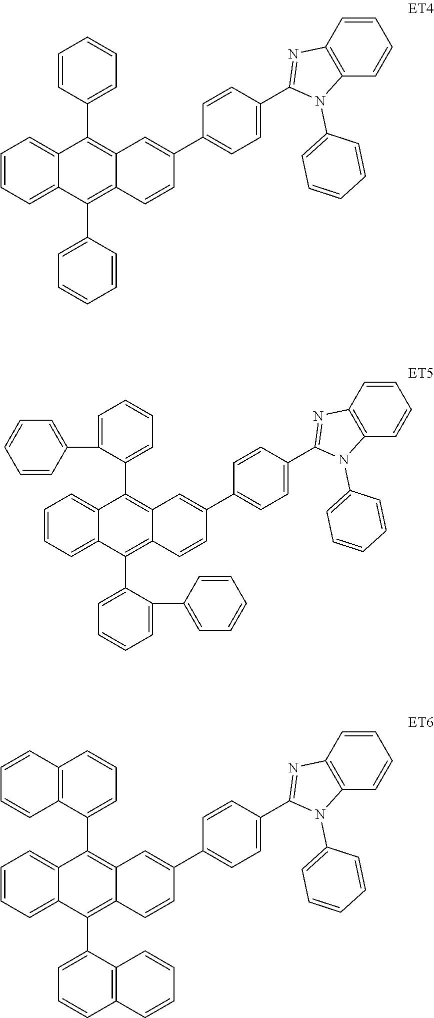 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

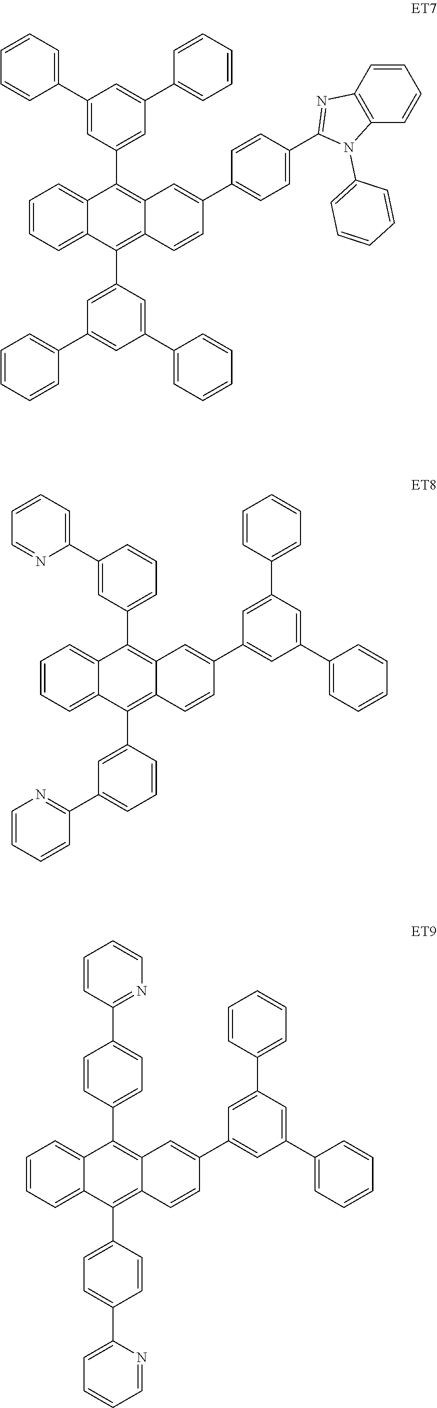 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

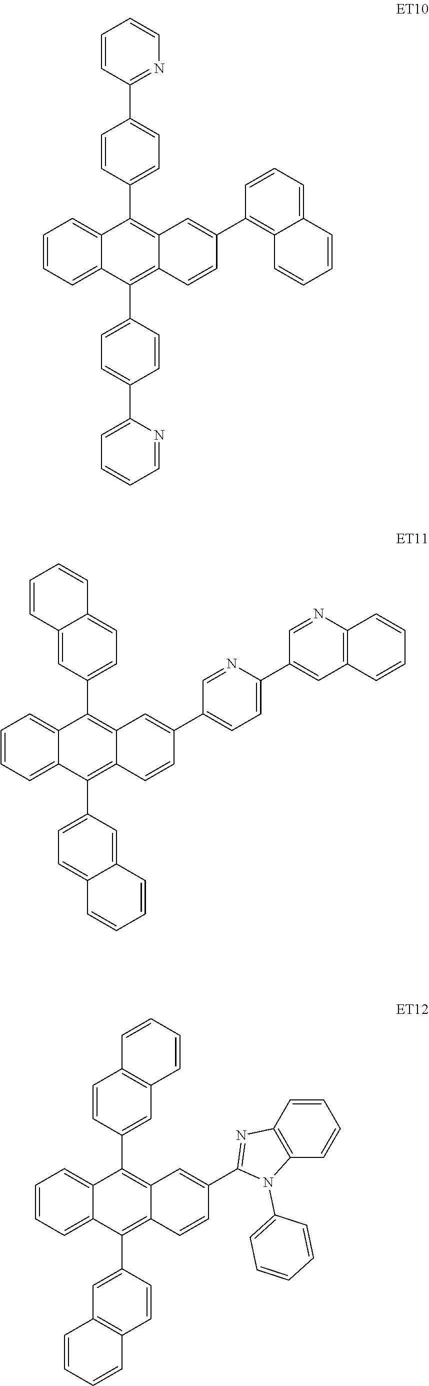 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

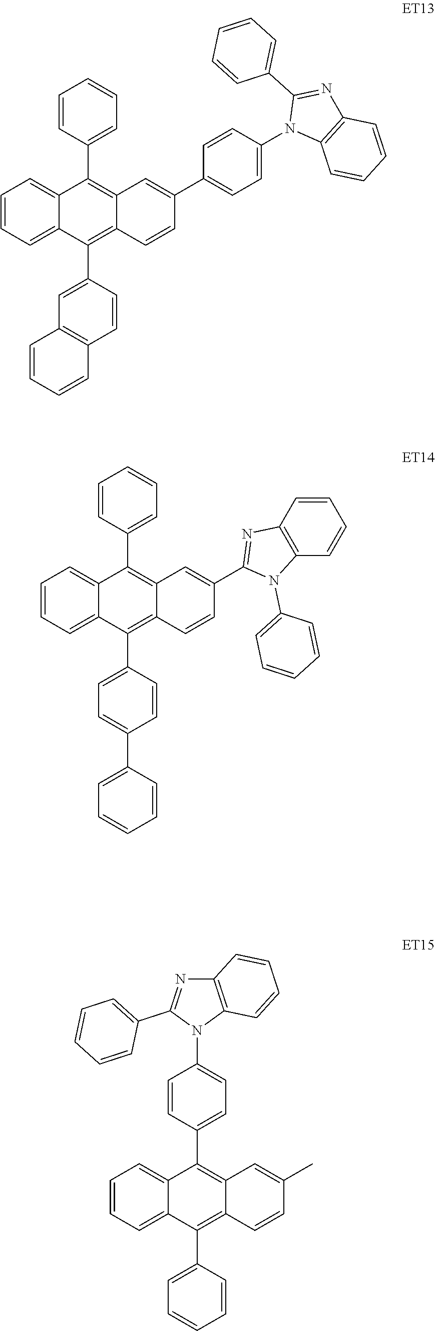 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

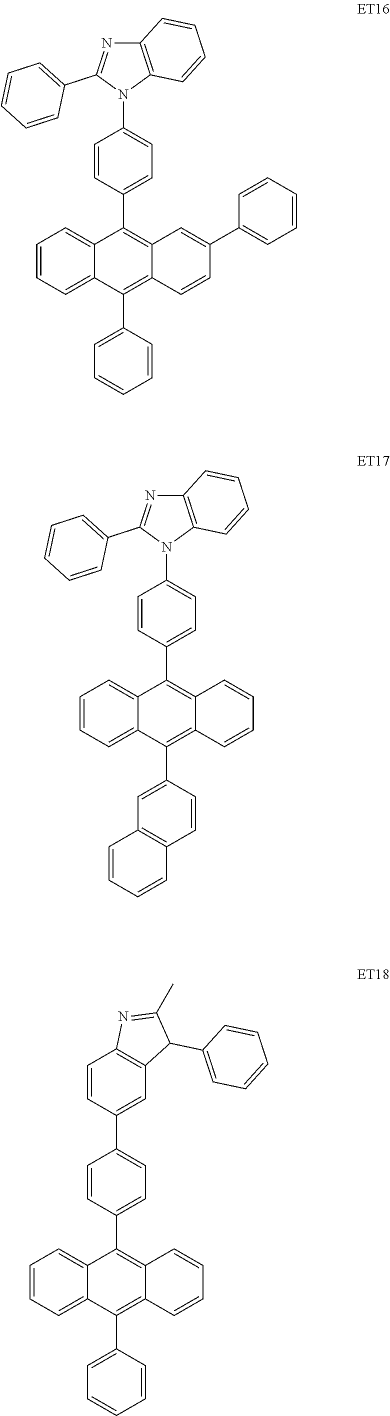 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; CC1=Nc2ccc(-c3ccc(-c4c5ccccc5c(-c5ccccc5)c5ccccc45)cc3)cc2C1c1ccccc1]

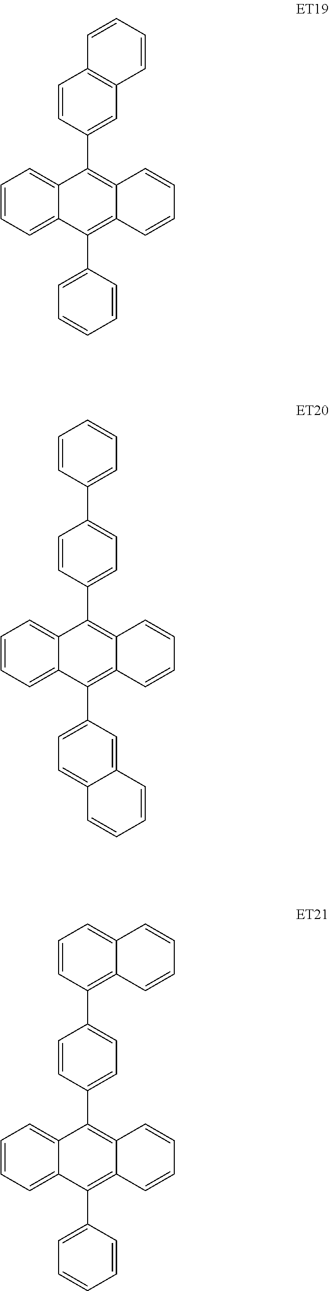

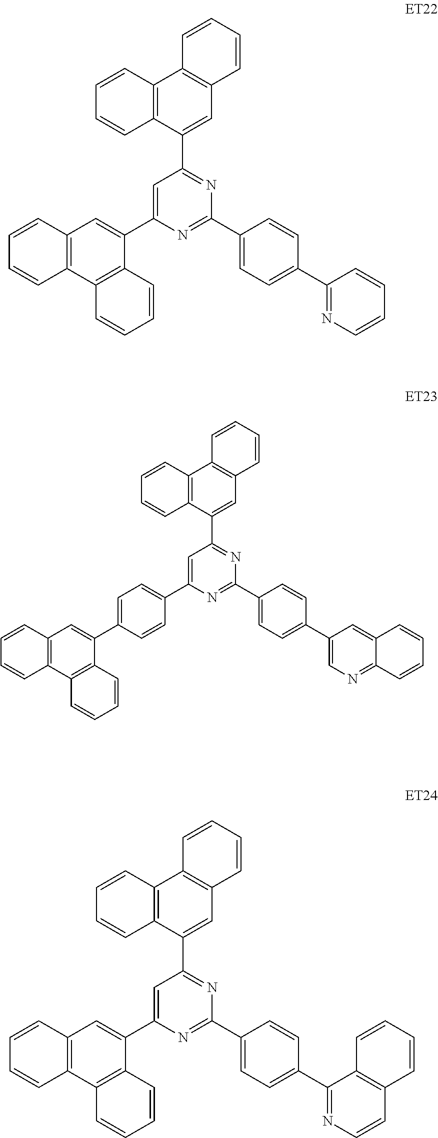 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

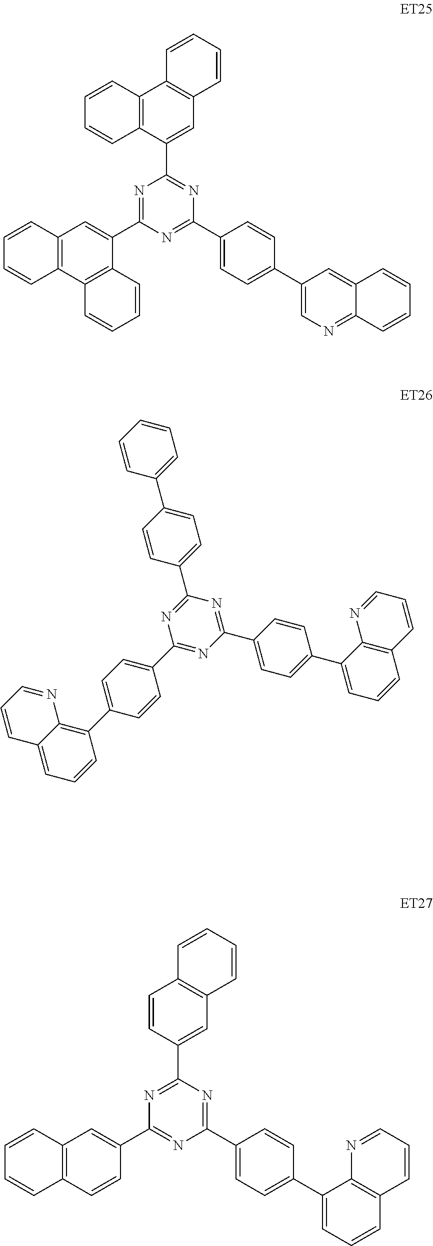 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

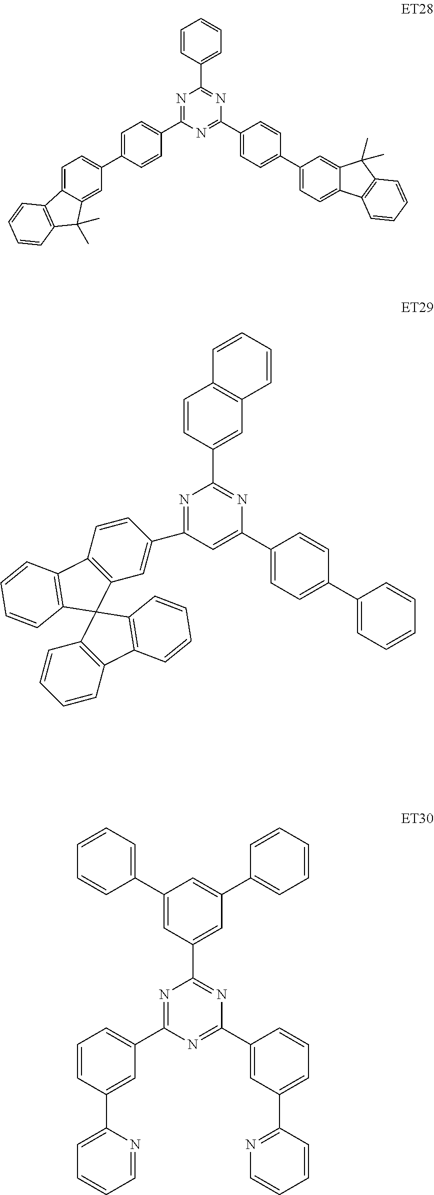 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

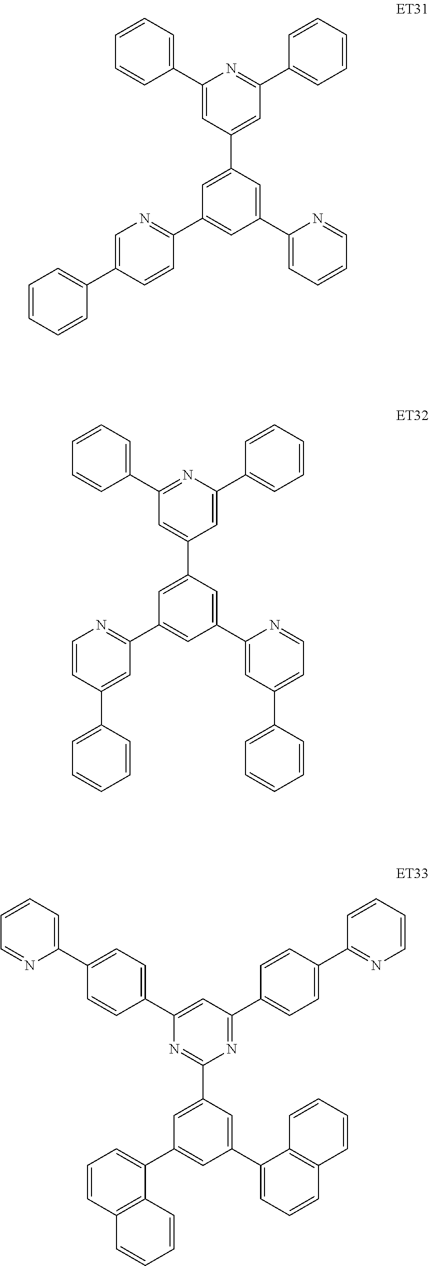 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

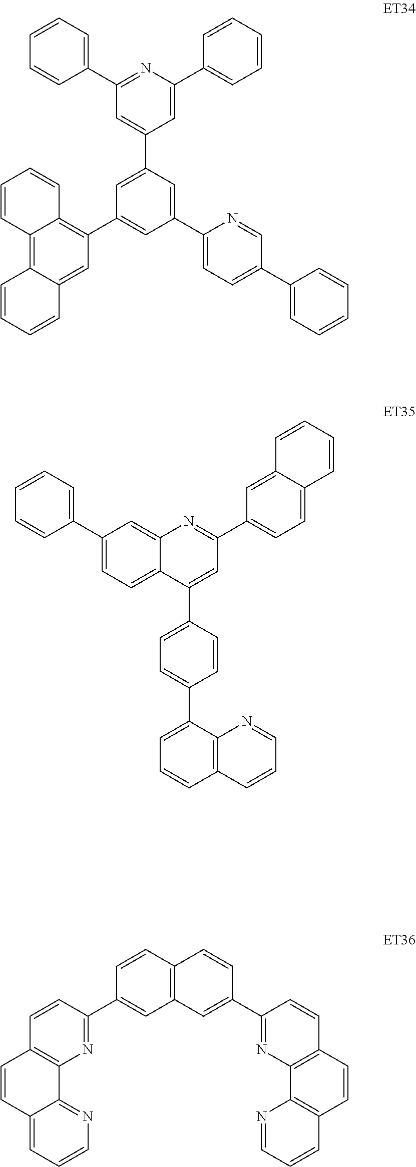 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

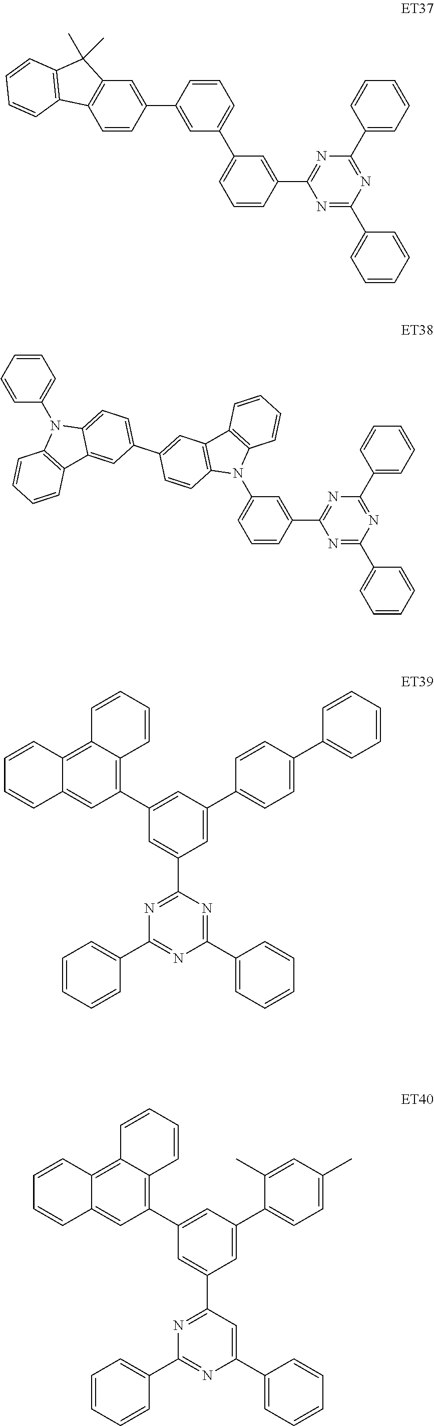 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

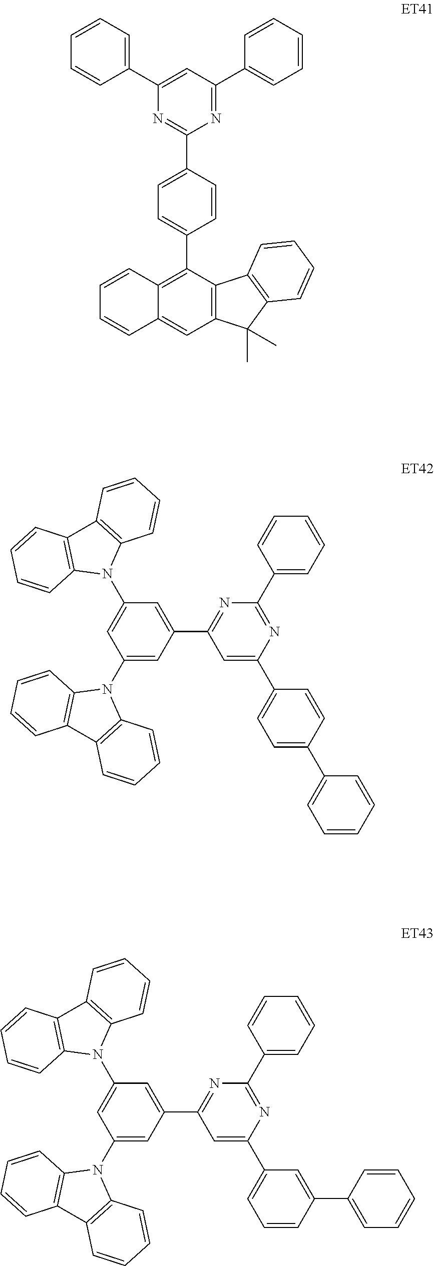 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

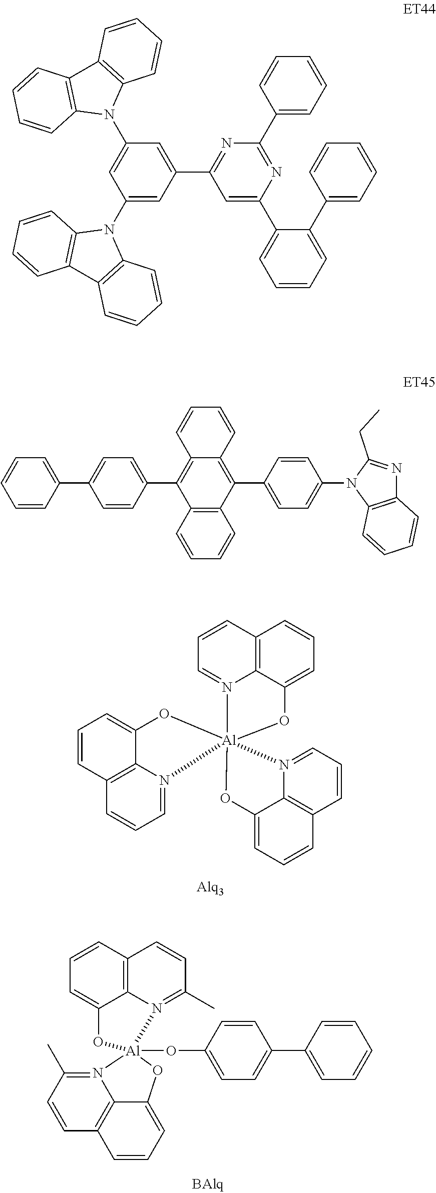

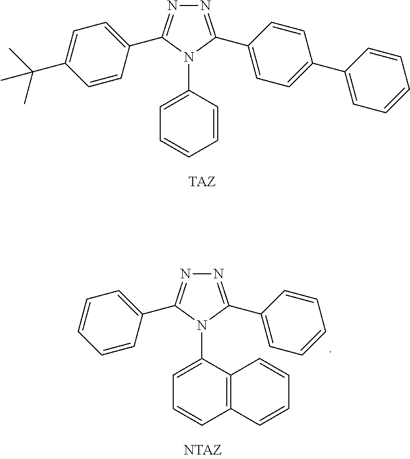 [SMILES: CC(C)(C)c1ccc(-c2nnc(-c3ccc(-c4ccccc4)cc3)n2-c2ccccc2)cc1 ; c1ccc(-c2nnc(-c3ccccc3)n2-c2cccc3ccccc23)cc1]

A thickness of the electron transport region may be in a range of about 100 Å to about 5,000 Å, and in some embodiments, about 160 Å to about 4,000 Å. When the electron transport region includes a buffer layer, a hole blocking layer, an electron control layer, an electron transport layer, or a combination thereof, the thicknesses of the buffer layer, the hole blocking layer, and/or the electron control layer may each independently be in a range of about 20 Å to about 1,000 Å, for example, about 30 Å to about 300 Å, and the thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region are each within these ranges, excellent or suitable electron transport characteristics may be obtained without a substantial increase in driving voltage.

In one or more embodiments, the electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a lithium (Li) ion, a sodium (Na) ion, a potassium (K) ion, a rubidium (Rb) ion, or a cesium (Cs) ion. A metal ion of the alkaline earth metal complex may be a beryllium (Be) ion, a magnesium (Mg) ion, a calcium (Ca) ion, a strontium (Sr) ion, or a barium (Ba) ion. Each ligand coordinated with the metal ion of the alkali metal complex or the alkaline earth metal complex may independently be hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, in some embodiments, the metal-containing material may include a Li complex. The Li complex may include, e.g., Compound ET-D1 (LiQ) or Compound ET-D2:

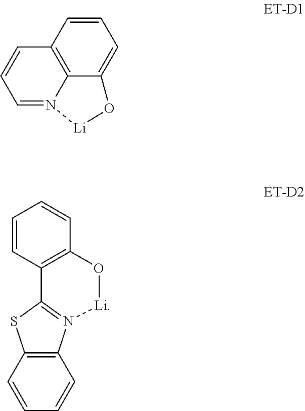

In one or more embodiments, the electron transport region may include an electron injection layer that facilitates injection of electrons from the second electrode 150. The electron injection layer may be in direct contact with the second electrode 150.

The electron injection layer may have i) a single-layered structure including (e.g., consisting of) a single layer including (e.g., consisting of) a single material, ii) a single-layered structure including (e.g., consisting of) a single layer including a plurality of different materials, or iii) a multi-layered structure having a plurality of layers including a plurality of different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may be Li, Na, K, Rb, Cs or any combination thereof. The alkaline earth metal may be Mg, Ca, Sr, Ba, or any combination thereof. The rare earth metal may be Sc, Y, Ce, Tb, Yb, Gd, or any combination thereof.

The alkali metal-containing compound, the alkaline earth metal-containing compound, and the rare earth metal-containing compound may respectively be oxides, halides (e.g., fluorides, chlorides, bromides, or iodides), tellurides, or any combination thereof of each of the alkali metal, the alkaline earth metal, and the rare earth metal.

The alkali metal-containing compound may be alkali metal oxides such as Li₂O, Cs₂O, and/or K₂O, alkali metal halides such as LiF, NaF, CsF, KF, Lil, Nal, Csl, and/or KI, or any combination thereof. The alkaline earth-metal-containing compound may include alkaline earth-metal oxides, such as BaO, SrO, CaO, BaₓSr₁₋ₓO (wherein x is a real number satisfying 0<x<1), and/or BaₓCa₁₋ₓO (wherein x is a real number satisfying 0<x<1). The rare earth metal-containing compound may include YbF₃, ScF₃, Sc₂O₃, Y₂O₃, Ce₂O₃, GdF₃, TbF₃, Ybl₃, Scl₃, Tbl₃, or any combination thereof. In some embodiments, the rare earth metal-containing compound may include a lanthanide metal telluride. Non-limiting examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth metal complex, and the rare earth metal complex may include: i) one of ions of the alkali metal, alkaline earth metal, and rare earth metal described above, respectively, and ii) a ligand bond to the metal ion, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

In one or more embodiments, the electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In some embodiments, the electron injection layer may further include an organic material (e.g., a compound represented by Formula 601).

In some embodiments, the electron injection layer may include (e.g., consist of) i) an alkali metal-containing compound (e.g., alkali metal halide), or ii) a) an alkali metal-containing compound (e.g., alkali metal halide); and b) an alkali metal, an alkaline earth metal, a rare earth metal, or any combination thereof. In some embodiments, the electron injection layer may be a KI:Yb co-deposition layer, a Rbl:Yb co-deposition layer, a LiF:Yb co-deposition layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth metal complex, the rare earth metal complex, or any combination thereof may be substantially homogeneously or non-homogeneously dispersed in a matrix including the organic material.

A thickness of the electron injection layer may be in a range of about 1 Å to about 100 Å, and in some embodiments, about 3 Å to about 90 Å. When the thickness of the electron injection layer is within these ranges, excellent or suitable electron injection characteristics may be obtained without a substantial increase in driving voltage.

Second electrode 150

The second electrode 150 may be on the interlayer 130. In one or more embodiments, the second electrode 150 may be a cathode that is an electron injection electrode. In those embodiment, a material for forming the second electrode 150 may be a material having a low work function, for example, a metal, an alloy, an electrically conductive compound, or a combination thereof, each having a low work function.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure, or a multi-layered structure including two or more layers.

### Capping Layer

A first capping layer may be located outside the first electrode 110, and/or a second capping layer may be located outside the second electrode 150. In some embodiments, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

In some embodiments, in the light-emitting device 10, light emitted from the emission layer in the interlayer 130 may pass through the first electrode 110 (which may be a semi-transmissive electrode or a transmissive electrode) and through the first capping layer to the outside. In some embodiments, in the light-emitting device 10, light emitted from the emission layer in the interlayer 130 may pass through the second electrode 150 (which may be a semi-transmissive electrode or a transmissive electrode) and through the second capping layer to the outside.

The first capping layer and the second capping layer may improve the external luminescence efficiency based on the principle of constructive interference. Accordingly, the optical extraction efficiency of the light-emitting device 10 may be increased, thus improving the luminescence efficiency of the light-emitting device 10.

The first capping layer and the second capping layer may each include a material having a refractive index of 1.6 or higher (e.g., at 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from among the first capping layer and the second capping layer may (e.g., the first capping layer and the second capping layer may each) independently include carbocyclic compounds, heterocyclic compounds, amine group-containing compounds, porphine derivatives, phthalocyanine derivatives, naphthalocyanine derivatives, alkali metal complexes, alkaline earth metal complexes, or any combination thereof. The carbocyclic compound, the heterocyclic compound, and the amine group-containing compound may optionally be substituted with a substituent including O, N, S, Se, Si, F, Cl, Br, I, or any combination thereof. In some embodiments, at least one selected from among the first capping layer and the second capping layer may (e.g., the first capping layer and the second capping layer may each) independently include an amine group-containing compound.

In some embodiments, at least one selected from among the first capping layer and the second capping layer may (e.g., the first capping layer and the second capping layer may each) independently include a compound represented by Formula 201, a compound represented by Formula 202, or any combination thereof.

In one or more embodiments, at least one selected from among the first capping layer and the second capping layer may (e.g., the first capping layer and the second capping layer may each) independently include at least one selected from among Compounds HT28 to HT33, Compounds CP1 to CP6, β-NPB, and/or any combination thereof:

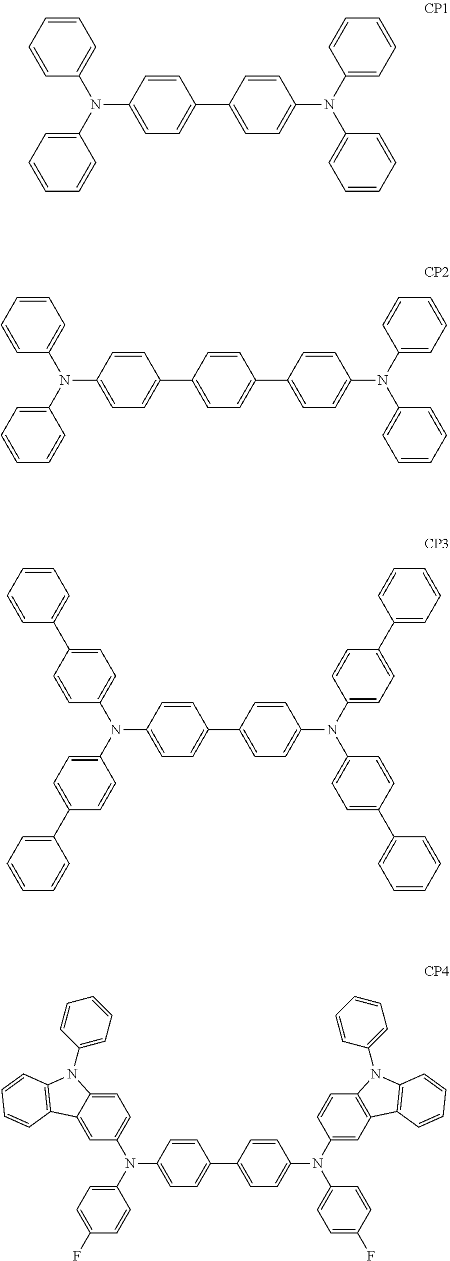 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

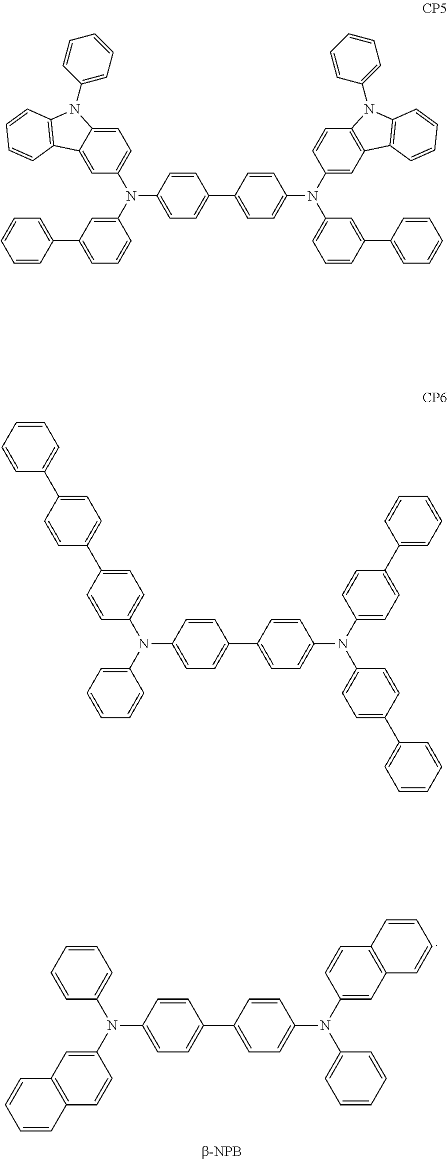 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The amine-containing compound represented by Formula 1 may be included in one or more suitable films. According to one or more embodiments of the present disclosure, a film including the amine-containing compound represented by Formula 1 may be provided. The film may be, for example, an optical member (or, a light-controlling member) (e.g., a color filter, a color-conversion member, a capping layer, a light extraction efficiency improvement layer, a selective light-absorbing layer, a polarizing layer, a quantum dot-containing layer, and/or the like), alight-blocking member (e.g., a light reflection layer or a light-absorbing layer), or a protection member (e.g., an insulating layer or a dielectric material layer).

### Electronic Apparatus

The light-emitting device may be included in one or more suitable electronic apparatuses. In some embodiments, an electronic apparatus including the light-emitting device may be a light-emitting apparatus or an authentication apparatus.

In one or more embodiments, the electronic apparatus (e.g., a light-emitting apparatus) may further include, in addition to the light-emitting device, i) a color filter, ii) a color conversion layer, or iii) a color filter and a color conversion layer. The color filter and/or the color conversion layer may be disposed on at least one travel direction of light emitted from the light-emitting device. For example, in some embodiments, light emitted from the light-emitting device may be blue light or white light (e.g., combined white light). The light-emitting device may be understood by referring to the descriptions provided herein. In some embodiments, the color conversion layer may include quantum dots. The quantum dot may be, for example, the quantum dot described herein.

The electronic apparatus may include a first substrate. The first substrate may include a plurality of sub-pixel areas, the color filter may include a plurality of color filter areas respectively corresponding to the plurality of sub-pixel areas, and the color conversion layer may include a plurality of color conversion areas respectively corresponding to the plurality of sub-pixel areas.

A pixel-defining film may be located between the plurality of sub-pixel areas to define each sub-pixel area.

The color filter may further include a plurality of color filter areas and light-blocking patterns between the plurality of color filter areas, and the color conversion layer may further include a plurality of color conversion areas and light-blocking patterns between the plurality of color conversion areas.

The plurality of color filter areas (or a plurality of color conversion areas) may include: a first area to emit first color light; a second area to emit second color light; and/or a third area to emit third color light, and the first color light, the second color light, and/or the third color light may have different maximum emission wavelengths. In some embodiments, the first color light may be red light, the second color light may be green light, and the third color light may be blue light. In some embodiments, the plurality of color filter areas (or the plurality of color conversion areas) may each include quantum dots. In some embodiments, the first area may include red quantum dots to emit red light, the second area may include green quantum dots to emit green light, and the third area may not include (e.g., may exclude) a quantum dot. The quantum dot may be understood by referring to the description of the quantum dot provided herein. The first area, the second area, and/or the third area may each further include an emitter.

In some embodiments, the light-emitting device may be to emit first light, the first area may be to absorb the first light to emit first-first color light, the second area may be to absorb the first light to emit second-first color light, and the third area may be to absorb the first light to emit third-first color light. In these embodiments, the first-first color light, the second-first color light, and the third-first color light may each have a different maximum emission wavelength. In some embodiments, the first light may be blue light, the first-first color light may be red light, the second-first color light may be green light, and the third-first light may be blue light.

In one or more embodiments, the electronic apparatus may further include a thin-film transistor, in addition to the light-emitting device. The thin-film transistor may include a source electrode, a drain electrode, and an active layer, wherein one selected from the source electrode and the drain electrode may be electrically connected to the first electrode or the second electrode of the light-emitting device.

The thin-film transistor may further include a gate electrode, a gate insulating film, and/or the like.

The active layer may include a crystalline silicon, an amorphous silicon, an organic semiconductor, and/or an oxide semiconductor.

In one or more embodiments, the electronic apparatus may further include an encapsulation unit for sealing the light-emitting device. The encapsulation unit may be located between the color filter and/or the color conversion layer and the light-emitting device. The encapsulation unit may allow light to pass to the outside from the light-emitting device and prevent or reduce the air and moisture to permeate to the light-emitting device at the same time. The encapsulation unit may be a sealing substrate including transparent glass or a plastic substrate. The encapsulation unit may be a thin-film encapsulating layer including at least one of an organic layer and/or an inorganic layer. When the encapsulation unit is a thin-film encapsulating layer, the electronic apparatus may be flexible.

In addition to the color filter and/or the color conversion layer, one or more suitable functional layers may be disposed on the encapsulation unit depending on the utilization of the electronic apparatus. Non-limiting examples of the functional layer may include a touch screen layer, a polarizing layer, and/or the like. The touch screen layer may be a resistive touch screen layer, a capacitive touch screen layer, or an infrared beam touch screen layer. The authentication apparatus may be, for example, a biometric authentication apparatus that identifies an individual according to biometric information (e.g., a fingertip, a pupil, and/or the like).

The authentication apparatus may further include a biometric information collecting unit, in addition to the light-emitting device described above.

The electronic apparatus may be applicable to one or more suitable displays, an optical source, lighting, a personal computer (e.g., a mobile personal computer), a cellphone, a digital camera, an electronic note, an electronic dictionary, an electronic game console, a medical device (e.g., an electronic thermometer, a blood pressure meter, a glucometer, a pulse measuring device, a pulse wave measuring device, an electrocardiograph recorder, an ultrasonic diagnosis device, or an endoscope display device), a fish finder, one or more suitable measurement devices, gauges (e.g., gauges of an automobile, an airplane, or a ship), and/or a projector.

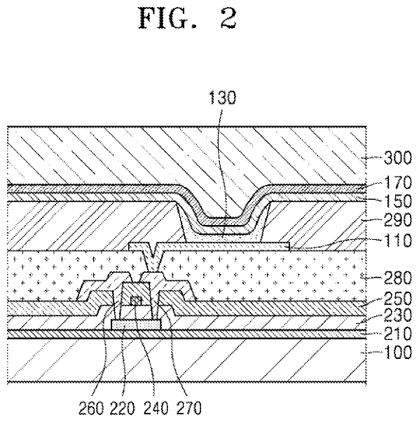

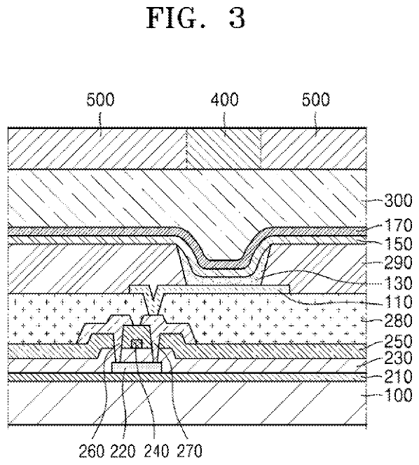

### Descriptions of FIGS. 2 and 3

FIG. 2 is a schematic cross-sectional view of an electronic apparatus according to one or more embodiments of the present disclosure.

The electronic apparatus shown in FIG. 2 may include a substrate 100, a thin-film transistor, a light-emitting device, and an encapsulation unit 300 sealing the light-emitting device.

The substrate 100 may be a flexible substrate, a glass substrate, or a metal substrate. A buffer layer 210 may be on the substrate 100. The buffer layer 210 may prevent or reduce penetration of impurities through the substrate 100 and provide a flat surface on the substrate 100.

A thin-film transistor may be on the buffer layer 210. The thin-film transistor may include an active layer 220, a gate electrode 240, a source electrode 260, and a drain electrode 270.

The active layer 220 may include an inorganic semiconductor such as silicon or polysilicon, an organic semiconductor, or an oxide semiconductor and may include a source area, a drain area, and a channel area.

A gate insulating film 230 for insulating the active layer 220 and the gate electrode 240 may be on the active layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270 to provide insulation therebetween.

The source electrode 260 and the drain electrode 270 may be on the interlayer insulating film 250. The interlayer insulating film 250 and the gate insulating film 230 may be formed to expose the source area and the drain area of the active layer 220, and the source electrode 260 and the drain electrode 270 may be adjacent to (e.g., in contact with) the exposed source area and the exposed drain area of the active layer 220, respectively.

The thin-film transistor may be electrically connected to a light-emitting device to drive the light-emitting device and may be protected by a passivation layer 280. The passivation layer 280 may include an inorganic insulating film, an organic insulating film, or a combination thereof. A light-emitting device may be on the passivation layer 280. The light-emitting device may include a first electrode 110, an interlayer 130, and a second electrode 150.

The first electrode 110 may be on the passivation layer 280. The passivation layer 280 may not fully cover the drain electrode 270 and expose a specific area of the drain electrode 270, and the first electrode 110 may be disposed to connect to the exposed area of the drain electrode 270.

A pixel-defining film 290 may be on the first electrode 110. The pixel-defining film 290 may expose a specific area of the first electrode 110, and the interlayer 130 may be formed in the exposed area of the first electrode 110. The pixel-defining film 290 may be a polyimide or polyacryl organic film. In some embodiments, at least some layers of the interlayer 130 may extend to the upper portion of the pixel-defining film 290 to be located in the form of a common layer.

The second electrode 150 may be on the interlayer 130, and a capping layer 170 may be additionally formed on the second electrode 150. The capping layer 170 may be formed to cover the second electrode 150.

The encapsulation unit 300 may be on the capping layer 170. The encapsulation unit 300 may be on the light-emitting device to protect the light-emitting device from moisture and/or oxygen. The encapsulation unit 300 may include: an inorganic film including silicon nitride (SiNₓ), silicon oxide (SiOₓ), indium tin oxide, indium zinc oxide, or any combination thereof; an organic film including PET, polyethylene naphthalate, polycarbonate, polyimide, polyethylene sulfonate, polyoxy methylene, poly arylate, hexamethyl disiloxane, an acrylic resin (e.g., polymethyl methacrylate, polyacrylic acid, and/or the like), an epoxy resin (e.g., aliphatic glycidyl ether (AGE) and/or the like), or any combination thereof; or a combination of the inorganic film(s) and/or the organic film(s).

FIG. 3 is a schematic cross-sectional view of an electronic apparatus according to one or more embodiments of the present disclosure.

The electronic apparatus shown in FIG. 3 may be substantially identical to the electronic apparatus shown in FIG. 2, except that a light-shielding pattern 500 and a functional area 400 are additionally located on the encapsulation unit 300. The functional area 400 may be i) a color filter area, ii) a color-conversion area, or iii) a combination of a color filter area and a color-conversion area. In some embodiments, the light-emitting device shown in FIG. 3 included in the electronic apparatus may be a tandem light-emitting device.

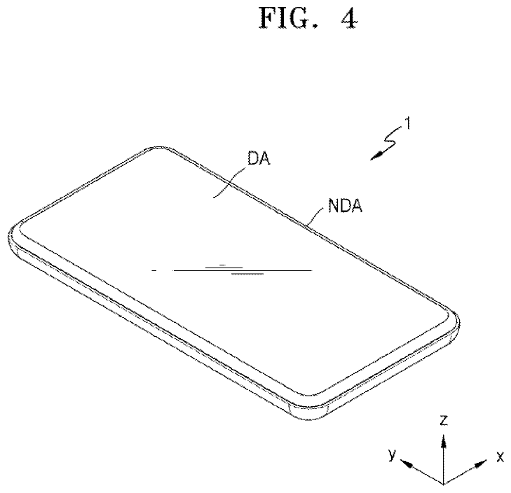

### Description of FIG. 4

FIG. 4 is a perspective view schematically illustrating electronic equipment 1 including the light-emitting device according to one or more embodiments of the present disclosure. The electronic equipment 1 may be an apparatus for displaying a moving image or still image, and may be any product such as a television, a laptop, a monitor, a billboard, and/or internet of things (IOT), and/or a portable electronic device such as a mobile phone, a smart phone, a tablet personal computer (PC), a mobile communication terminal, an electronic notebook, an electronic book, and/or a portable multimedia player (PMP) and/or a navigation device, an ultra-mobile PC (UMPC), or a part thereof. In some embodiments, the electronic equipment 1 may be a wearable device such as a smart watch, a watch phone, a glasses display, and/or a head mounted display (HMD), or a part thereof, but embodiments of the present disclosure are not limited thereto. For example, in some embodiments, the electronic equipment 1 may be a center information display (CID) on an instrument panel and a center fascia or dashboard of a vehicle, a room mirror display instead of a side mirror of a vehicle, an entertainment display for the rear seat of a car or a display placed on the back of the front seat, head up display (HUD) installed in the front of a vehicle or projected on a front window glass, and/or a computer generated hologram augmented reality head up display (CGH AR HUD). FIG. 4 shows an embodiment where the electronic equipment 1 is a smart phone for convenience of description.

The electronic equipment 1 shown in FIG. 4 may include a display area DA and a non-display area NDA outside the display area DA. A display apparatus of the electronic equipment 1 may realize an image through an array of a plurality of pixels that are two-dimensionally arranged in the display area DA.

The non-display area NDA may be an area that may not display an image, and may completely surround the display area DA. In the non-display area NDA, a driver for providing an electrical signal or power to the display devices arranged in the display area DA may be arranged. In the non-display area NDA, a pad, which is an area to which an electronic device or a printed circuit board may be electrically connected, may be arranged.

The electronic equipment 1 may have different lengths in the x-axis direction and in the y-axis direction. For example, in some embodiments, as shown in FIG. 4, the length in the x-axis direction may be shorter than the length in the y-axis direction. In some embodiments, the length in the x-axis direction may be the same as the length in the y-axis direction. In some embodiments, the length in the x-axis direction may be longer than the length in the y-axis direction.

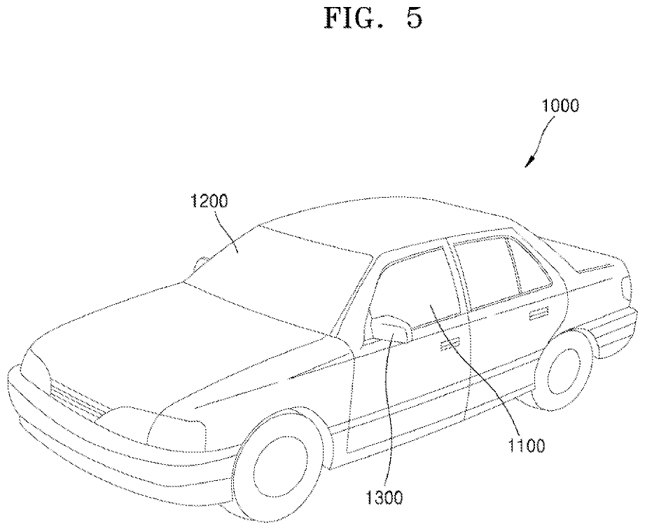

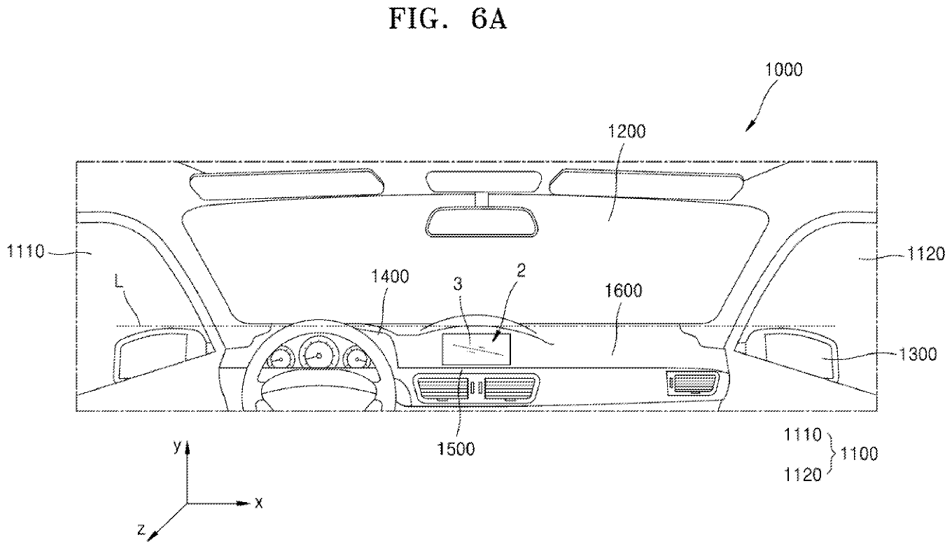

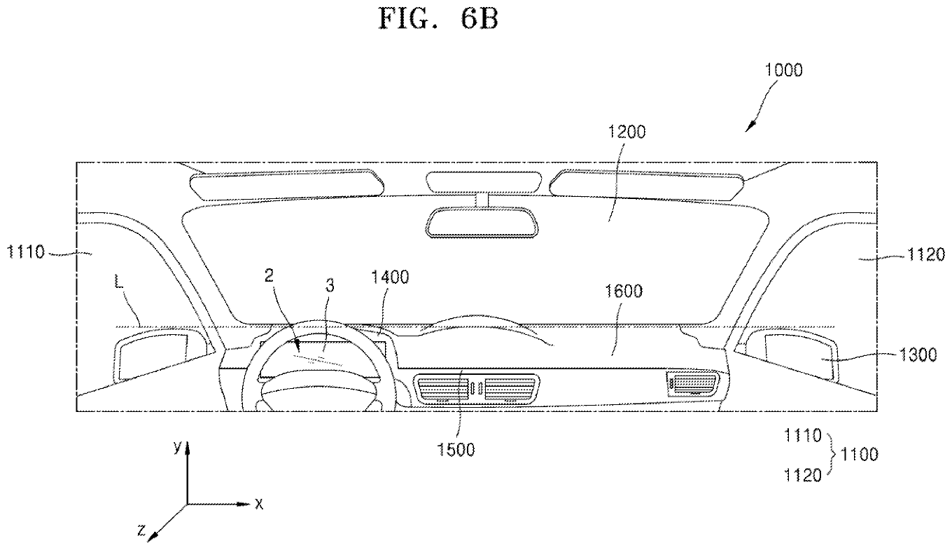

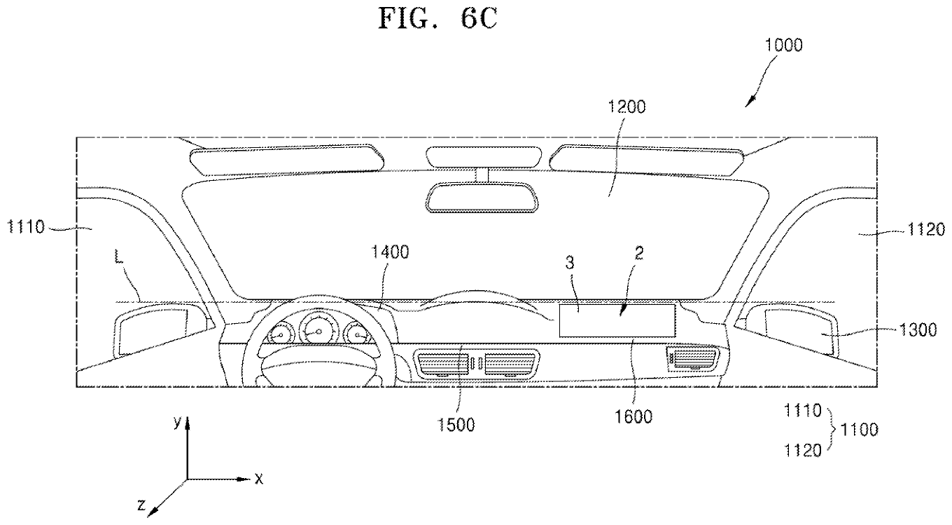

### Descriptions of FIGS. 5 and 6A to 6C

FIG. 5 is a schematic view illustrating an exterior of a vehicle 1000 as electronic equipment including a light-emitting device according to one or more embodiments of the present disclosure. FIGS. 6A to 6C are each a schematic view illustrating an interior of the vehicle 1000 according to one or more embodiments of the present disclosure.

In FIGS. 5 and 6A to 6C, the vehicle 1000 may refer to one or more suitable apparatuses that move an object to be transported such as a human, an object, or an animal, from a departure point to a destination. The vehicle 1000 may include a vehicle traveling on a road or a track, a vessel moving over the sea or a river, and an airplane flying in the sky utilizing the action of air.

The vehicle 1000 may travel on roads or tracks. The vehicle 1000 may move in a set or predetermined direction according to rotation of at least one wheel thereof. For example, the vehicle 1000 may include a three-wheeled or four-wheeled vehicle, a construction machine, a two-wheeled vehicle, a motorbike, a bicycle, or a train running on a track.

The vehicle 1000 may include a body having an interior and an exterior, and a chassis in which mechanical apparatuses necessary for driving are installed as the remaining parts except for the body. The exterior of the body of the vehicle may include a front panel, a bonnet, a roof panel, a rear panel, a trunk, and a pillar provided at a boundary between doors. The chassis of the vehicle 1000 may include a power generating apparatus, a power transmitting apparatus, a traveling apparatus, a steering apparatus, a braking apparatus, a suspension apparatus, a transmission apparatus, a fuel apparatus, front and rear, left and right wheels, and/or the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar located between the side window glass 1100 and the front window glass 1200.

The side window glass 1100 may be installed on a side of the vehicle 1000. In some embodiments, the side window glass 1100 may be installed on a door of the vehicle 1000. A plurality of side window glasses 1100 may be provided and may face each other. In some embodiments, the side window glass 1100 may include a first side window glass 1110 and a second side window glass 1120. In some embodiments, the first side window glass 1110 may be arranged adjacent to the cluster 1400. In some embodiments, the second side window glass 1120 may be arranged adjacent to the passenger seat dashboard 1600.

In some embodiments, the side window glasses 1100 may be spaced apart from each other in the x direction or the −x direction. For example, the first side window glass 1110 and the second side window glass 1120 may be spaced apart from each other in the x direction or the −x direction. In other words, an imaginary straight line L connecting the side window glasses 1100 may extend in the x direction or the −x direction. For example, the imaginary straight line L connecting the first side window glass 1110 and the second side window glass 1120 to each other may extend in the x direction or the −x direction.

The front window glass 1200 may be installed on a front of the vehicle 1000. The front window glass 1200 may be arranged between the side window glasses 1100 facing each other.

The side mirror 1300 may provide a rear view of the vehicle 1000. The side mirror 1300 may be installed on the exterior of the body of the vehicle. In some embodiments, a plurality of side mirrors 1300 may be provided. Any one of the plurality of side mirrors 1300 may be located outside the first side window glass 1110. The other one of the plurality of side mirrors 1300 may be located outside the second side window glass 1120.

The cluster 1400 may be located in front of the steering wheel. The cluster 1400 may include a tachometer, a speedometer, a coolant thermometer, a fuel gauge turn indicator, a high beam indicator, a warning indicator, a seat belt warning indicator, an odometer, a hodometer, an automatic shift selector indicator, a door open warning indicator, an engine oil warning indicator, and/or a low fuel warning indicator.

The center fascia 1500 may include a control panel on which a plurality of buttons for adjusting an audio apparatus, an air conditioning apparatus, and/or a heater of seats. The center fascia 1500 may be on one side of the cluster 1400.

The passenger seat dashboard 1600 may be spaced apart from the cluster 1400 with the center fascia 1500 interposed therebetween. In some embodiments, the cluster 1400 may be disposed to correspond to a driver seat, and the passenger seat dashboard 1600 may be disposed to correspond to a passenger seat. In some embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be inside the vehicle 1000. In some embodiments, the display apparatus 2 may be arranged between the side window glasses 1100 facing each other. The display apparatus 2 may be on at least one selected from among the cluster 1400, the center fascia 1500, and the passenger seat dashboard 1600.

The display apparatus 2 may include an organic light-emitting display apparatus, an inorganic EL display apparatus, a quantum dot display apparatus, and/or the like. Hereinafter, as the display apparatus 2 according to one or more embodiments, an organic light-emitting display apparatus including the light-emitting device according to one or more embodiments will be described as an example, however, embodiments of the present disclosure may include one or more suitable types (kinds) of the display apparatus.

As shown in FIG. 6A, in some embodiments, the display apparatus 2 may be disposed on the center fascia 1500. In one embodiment, the display apparatus 2 may display navigation information. In one embodiment, the display apparatus 2 may display information of audio, video, and/or vehicle settings.

As shown in FIG. 6B, in some embodiments, the display apparatus 2 may be disposed on the cluster 1400. In these embodiments, the cluster 1400 may show driving information and/or the like by the display apparatus 2. For example, the cluster 1400 may be implemented digitally. The digital cluster 1400 may display vehicle information and driving information as images. For example, a needle and a gauge and one or more suitable warning indicators of a tachometer may be displayed by digital signals.

As shown in FIG. 6C, in some embodiments, the display apparatus 2 may be disposed on the passenger seat dashboard 1600. The display apparatus 2 may be embedded in the passenger seat dashboard 1600 or located on the passenger seat dashboard 1600. In some embodiments, the display apparatus 2 disposed on the passenger seat dashboard 1600 may display an image related to information displayed on the cluster 1400 and/or information displayed on the center fascia 1500. In some embodiments, the display apparatus 2 disposed on the passenger seat dashboard 1600 may display information different from information displayed on the cluster 1400 and/or information displayed on the center fascia 1500.

### Manufacturing Method

The layers constituting the hole transport region, the emission layer, and the layers constituting the electron transport region may be formed in a specific region by utilizing one or more suitable methods such as vacuum deposition, spin coating, casting, Langmuir-Blodgett (LB) deposition, ink-jet printing, laser printing, and/or laser-induced thermal imaging.

When the layers constituting the hole transport region, the emission layer, and the layers constituting the electron transport region are each formed by vacuum deposition, the vacuum deposition may be performed at a deposition temperature in a range of about 100° C. to about 500° C. at a vacuum degree in a range of about 10⁻⁸ torr to about 10⁻³ torr, and at a deposition rate in a range of about 0.01 Angstroms per second (Å/sec) to about 100 Å/sec, depending on a material to be included in each layer and the structure of each layer to be formed.

### General Definitions of Terms

The term “C₃-C₆₀ carbocyclic group” as utilized herein refers to a cyclic group including (e.g., consisting of) carbon atoms only and having 3 to 60 carbon atoms as ring-forming atoms. The term “C₁-C₆₀ heterocyclic group” as utilized herein refers to a cyclic group having 1 to 60 carbon atoms in addition to a heteroatom as a ring-forming atom other than carbon atoms. The C₃-C₆₀ carbocyclic group and the C₁-C₆₀ heterocyclic group may each be a monocyclic group including (e.g., consisting of) one ring or a polycyclic group in which at least two rings are condensed. For example, the number of ring-forming atoms in the C₁-C₆₀ heterocyclic group may be in a range of 3 to 61.

The term “cyclic group” as utilized herein may include the C₃-C₆₀ carbocyclic group and the C₁-C₆₀ heterocyclic group.

The term “rr electron-rich C₃-C₆₀ cyclic group” refers to a cyclic group having 3 to 60 carbon atoms and not including *—N═*′ as a ring-forming moiety. The term “rr electron-deficient nitrogen-containing C₁-C₆₀ cyclic group” as utilized herein refers to a heterocyclic group having 1 to 60 carbon atoms and *—N═*′ as a ring-forming moiety.

In some embodiments,
the C₃-C₆₀ carbocyclic group may be i) a T1 group or ii) a group in which at least two T1 groups are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) a T2 group, ii) a group in which at least two T2 groups are condensed with each other, or iii) a group in which at least one T2 group is condensed with at least one T1 group (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
the π electron-rich C₃-C₆₀ cyclic group may be i) a T1 group, ii) a condensed group in which at least two T1 groups are condensed with each other, iii) a T3 group, iv) a condensed group in which at least two T3 groups are condensed with each other, or v) a condensed group in which at least one T3 group is condensed with at least one T1 group (for example, a C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonapthothiophene group, a benzonaphthosilole group, a benzofurodibenzofuran group, a benzofurodibenzothiophene group, a benzothienodibenzothiophene group, and/or the like), and
the π electron-deficient nitrogen-containing C₁-C₆₀ cyclic group may be i) a T4 group, ii) a group in which at least two T4 groups are condensed with each other, iii) a group in which at least one T4 group is condensed with at least one T1 group, iv) a group in which at least one T4 group is condensed with at least one T3 group, or v) a group in which at least one T4 group, at least one T1 group, and at least one T3 group are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),
wherein the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,
the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,
the T3 group may be a furan group, a thiophene group, a 1H-pyrrole group, a silole group, or a borole group, and
the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group”, “C₃-C₆₀ carbocyclic group”, “C₁-C₆₀ heterocyclic group”, “rr electron-rich C₃-C₆₀ cyclic group”, or “rr electron-deficient nitrogen-containing C₁-C₆₀ cyclic group” as utilized herein may be a group condensed with any suitable cyclic group, a monovalent group, or a polyvalent group (e.g., a divalent group, a trivalent group, a quadrivalent group, and/or the like), depending on the structure of a formula to which the term is applied. For example, a “benzene group” may be a benzo group, a phenyl group, a phenylene group, and/or the like, and this may be understood by one of ordinary skill in the art, depending on the structure of Formula including the “benzene group”.

Depending on context, in the present disclosure, a divalent group may refer or be a polyvalent group (e.g., trivalent, tetravalent, etc., and not just divalent) per, e.g., the structure of a formula in connection with which of the terms are utilized.

In some embodiments, non-limiting examples of the monovalent C₃-C₆₀ carbocyclic group and monovalent C₁-C₆₀ heterocyclic group may include a C₃-C₁₀ cycloalkyl group, a C₁-C₆₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group, and non-limiting examples of the divalent C₃-C₆₀ carbocyclic group and the divalent C₁-C₆₀ heterocyclic group may include a C₃-C₁₀ cycloalkylene group, a C₁-C₆₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group” as utilized herein refers to a linear or branched aliphatic hydrocarbon monovalent group having 1 to 60 carbon atoms, and non-limiting examples thereof may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an iso-nonyl group, a sec-nonyl group, a tert-nonyl group, an n-decyl group, an isodecyl group, a sec-decyl group, and/or a tert-decyl group. The term “C₁-C₆₀ alkylene group” as utilized herein refers to a divalent group having substantially the same structure as the C₁-C₆₀ alkyl group.

The term “C₂-C₆₀ alkenyl group” as utilized herein refers to a hydrocarbon group having at least one carbon-carbon double bond in the middle or at the terminus of the C₂-C₆₀ alkyl group. Non-limiting examples thereof may include an ethenyl group, a propenyl group, and a butenyl group. The term “C₂-C₆₀ alkenylene group” as utilized herein refers to a divalent group having substantially the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group. Non-limiting examples thereof may include an ethynyl group and/or a propynyl group. The term “C₂-C₆₀ alkynylene group” as utilized herein refers to a divalent group having substantially the same structure as the C₂-C₆₀ alkynyl group.

The term “C₁-C₆₀ alkoxy group” as utilized herein refers to a monovalent group represented by —OA₁₀₁ (wherein A₁₀₁ is a C₁-C₆₀ alkyl group). Non-limiting examples thereof may include a methoxy group, an ethoxy group, and/or an isopropyloxy group.

The term “C₃-C₁₀ cycloalkyl group” as utilized herein refers to a monovalent saturated hydrocarbon monocyclic group including 3 to 10 carbon atoms. Non-limiting examples of the C₃-C₁₀ cycloalkyl group as utilized herein may include a cyclopropyl group, a cyclobutyl group, a cyclopentyl group, a cyclohexyl group, a cycloheptyl group, a cyclooctyl group, an adamantanyl group, a norbornanyl (bicyclo[2.2.1]heptyl) group, a bicyclo[1.1.1]pentyl group, a bicyclo[2.1.1]hexyl group, and/or a bicyclo[2.2.2]octyl group. The term “C₃-C₁₀ cycloalkylene group” as utilized herein refers to a divalent group having substantially the same structure as the C₃-C₁₀ cycloalkyl group.

The term “C₁-C₁₀ heterocycloalkyl group” as utilized herein refers to a monovalent cyclic group including at least one heteroatom other than carbon atoms as a ring-forming atom and having 1 to 10 carbon atoms. Non-limiting examples thereof may include a 1,2,3,4-oxatriazolidinyl group, a tetrahydrofuranyl group, and/or a tetrahydrothiophenyl group. The term “C₁-C₆₀ heterocycloalkylene group” as utilized herein refers to a divalent group having substantially the same structure as the C₁-C₁₀ heterocycloalkyl group.

The term “C₃-C₁₀ cycloalkenyl group” as utilized herein refers to a monovalent cyclic group that has 3 to 10 carbon atoms and at least one carbon-carbon double bond in its ring, and is not aromatic. Non-limiting examples thereof may include a cyclopentenyl group, a cyclohexenyl group, and/or a cycloheptenyl group. The term “C₃-C₁₀ cycloalkenylene group” as utilized herein refers to a divalent group having substantially the same structure as the C₃-C₁₀ cycloalkenyl group.

The term “C₁-C₁₀ heterocycloalkenyl group” as utilized herein refers to a monovalent cyclic group including at least one heteroatom other than carbon atoms as a ring-forming atom, 1 to 10 carbon atoms, and at least one double bond in its ring. Non-limiting examples of the C₁-C₁₀ heterocycloalkenyl group may include a 4,5-dihydro-1,2,3,4-oxatriazolyl group, a 2,3-dihydrofuranyl group, and/or a 2,3-dihydrothiophenyl group. The term “C₁-C₁₀ heterocycloalkenylene group” as utilized herein refers to a divalent group having substantially the same structure as the C₁-C₁₀ heterocycloalkenyl group.

The term “C₆-C₆₀ aryl group” as utilized herein refers to a monovalent group having a carbocyclic aromatic system having 6 to 60 carbon atoms. The term “C₆-C₆₀ arylene group” as utilized herein refers to a divalent group having a carbocyclic aromatic system having 6 to 60 carbon atoms. Non-limiting examples of the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and/or an ovalenyl group. When the C₆-C₆₀ aryl group and the C₆-C₆₀ arylene group each independently include two or more rings, the respective rings may be fused.

The term “C₁-C₆₀ heteroaryl group” as utilized herein refers to a monovalent group having a heterocyclic aromatic system further including at least one heteroatom other than carbon atoms as a ring-forming atom and 1 to 60 carbon atoms. The term “C₁-C₆₀ heteroarylene group” as utilized herein refers to a divalent group having a heterocyclic aromatic system further including at least one heteroatom other than carbon atoms as a ring-forming atom and 1 to 60 carbon atoms. Non-limiting examples of the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and/or a naphthyridinyl group. When the C₁-C₆₀ heteroaryl group and the C₁-C₆₀ heteroarylene group each independently include two or more rings, the respective rings may be fused.

The term “monovalent non-aromatic condensed polycyclic group” as utilized herein refers to a monovalent group that has two or more rings condensed to each other and only carbon atoms (e.g., 8 to 60 carbon atoms) as ring forming atoms, wherein the molecular structure when considered as a whole is non-aromatic. Non-limiting examples of the monovalent non-aromatic condensed polycyclic group may include an indenyl group, a fluorenyl group, a spiro-bifluorenyl group, a benzofluorenyl group, an indenophenanthrenyl group, and/or an indenoanthracenyl group. The term “divalent non-aromatic condensed polycyclic group” as utilized herein refers to a divalent group having substantially the same structure as the monovalent non-aromatic condensed polycyclic group.

The term “monovalent non-aromatic condensed heteropolycyclic group” as utilized herein refers to a monovalent group that has two or more rings condensed to each other and at least one heteroatom other than carbon atoms (e.g., 1 to 60 carbon atoms), as a ring-forming atom, wherein the molecular structure when considered as a whole is non-aromatic. Non-limiting examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group” as utilized herein refers to a divalent group having substantially the same structure as the monovalent non-aromatic condensed heteropolycyclic group.

The term “C₆-C₆₀ aryloxy group” as utilized herein refers to —OA₁₀₂ (wherein A₁₀₂ is a C₆-C₆₀ aryl group). The term “C₆-C₆₀ arylthio group” as utilized herein refers to —SA₁₀₃ (wherein A₁₀₃ is a C₆-C₆₀ aryl group).

The term “C₇-C₆₀ aryl alkyl group” as utilized herein refers to -A₁₀₄A₁₀₅ (wherein A₁₀₄ is a C₁-C₅₄ alkylene group, and A₁₀₅ is a C₆-C₅₉ aryl group). The term “C₂-C₆₀ heteroaryl alkyl group” as utilized herein refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ” as utilized herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ aryl alkyl group; or a C₂-C₆₀ heteroaryl alkyl group.

The term “heteroatom” as utilized herein refers to any atom other than a carbon atom. Non-limiting examples of the heteroatom may include O, S, N, P, Si, B, Ge, Se, or any combination thereof.

A third-row transition metal as utilized herein may include hafnium (Hf), tantalum (Ta), tungsten (W), rhenium (Re), osmium (Os), iridium (Ir), platinum (Pt), and gold (Au).

The term “Ph” as utilized herein refers to a phenyl group. The term “Me” as utilized herein refers to a methyl group. The term “Et” as utilized herein refers to an ethyl group. The term “tert-Bu” or “Bu^{t}” as utilized herein refers to a tert-butyl group. The term “OMe” as utilized herein refers to a methoxy group.

The term “biphenyl group” as utilized herein refers to a phenyl group substituted with a phenyl group. In other words, the “biphenyl group” may be a substituted phenyl group having a C₆-C₆₀ aryl group as a substituent.

The term “terphenyl group” as utilized herein refers to a phenyl group substituted with a biphenyl group. In other words, the “terphenyl group” may be a substituted phenyl group having a C₆-C₆₀ aryl group substituted with a C₆-C₆₀ aryl group as a substituent.

The symbols * and *′ as utilized herein, unless defined otherwise, refer to a binding site to an adjacent atom in a corresponding formula or moiety.

In the present disclosure, the x-axis, y-axis, and z-axis are not limited to three axes on an orthogonal coordinates system, and may be interpreted in a broad sense including the orthogonal coordinates system. For example, the x-axis, y-axis, and z-axis may be orthogonal to each other, but the x-axis, y-axis, and z-axis may also refer to different directions that are not orthogonal to each other.

Hereinafter, compounds and a light-emitting device according to one or more embodiments will be described in more detail with reference to Synthesis Examples and Examples. The wording “B was utilized instead of A” utilized in describing Synthesis Examples refers to that an identical number of molar equivalents of B was utilized in place of A.

### EXAMPLE

### Synthesis Example 1 (Synthesis of Compound 1)

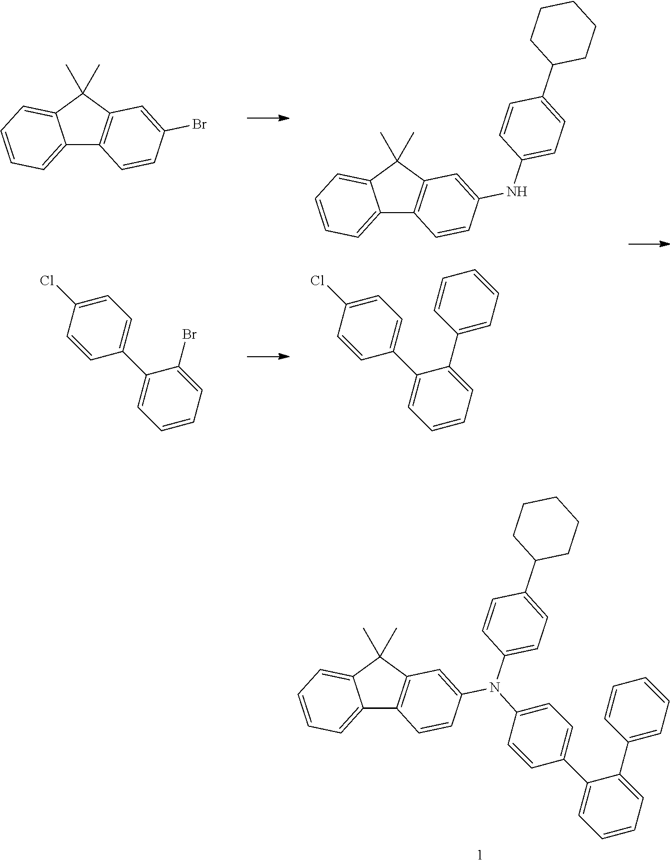

### Synthesis of Intermediate Compound 1-1

4-cyclohexylaniline (1.0 eq.), 2-bromo-9,9-dimethyl-9H-fluorene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 80° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 1-1 was obtained (yield: 85%).

### Synthesis of Intermediate Compound 1-2

phenylboronic acid (1.2 eq.), tetrakis(triphenylphosphine)palladium (0.05 eq.), and potassium carbonate (2.0 eq.) in 2-bromo-4′-chloro-1,1′-biphenyl (1.0 eq.) were dissolved in a solvent having a volumetric ratio of tetrahydrofuran (THF):H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 1-2 was obtained (yield: 85%).

### Synthesis of Compound 1

Intermediate Compound 1-1 (1.0 eq.), Intermediate Compound 1-2 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 1 was obtained (yield: 80%). By fast-atom bombardment mass spectrometry (FAB-MS), mass number m/z=595.32 was observed as a molecular ion peak. Thus, Compound 1 was identified.

### Synthesis Example 2 (Synthesis of Compound 2)

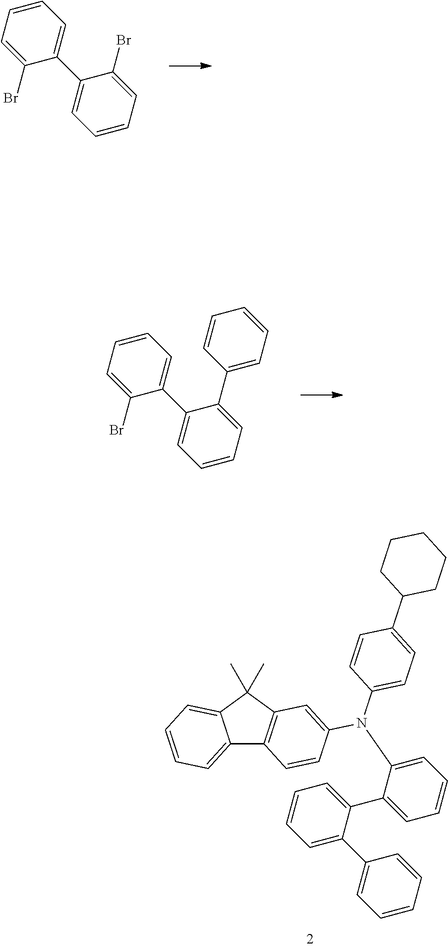 [SMILES: Brc1ccccc1-c1ccccc1Br ; Brc1ccccc1-c1ccccc1-c1ccccc1 ; CC1(C)c2ccccc2-c2ccc(N(c3ccc(C4CCCCC4)cc3)c3ccccc3-c3ccccc3-c3ccccc3)cc21]

### Synthesis of Intermediate Compound 2-1

phenylboronic acid (1.0 eq.), tetrakis(triphenylphosphine)palladium (0.05 eq.), and potassium carbonate (2.0 eq.) in 2,2′-dibromo-1,1′-biphenyl (1.0 eq.) were dissolved in a solvent having a volumetric ratio of THF:H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 2-1 was obtained (yield: 55%).

### Synthesis of Compound 2

Intermediate Compound 1-1 (1.0 eq.), Intermediate Compound 2-1 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 2 was obtained (yield: 80%). By FAB-MS, mass number m/z=595.32 was observed as a molecular ion peak. Thus, Compound 2 was identified.

### Synthesis Example 3 (Synthesis of Compound 3)

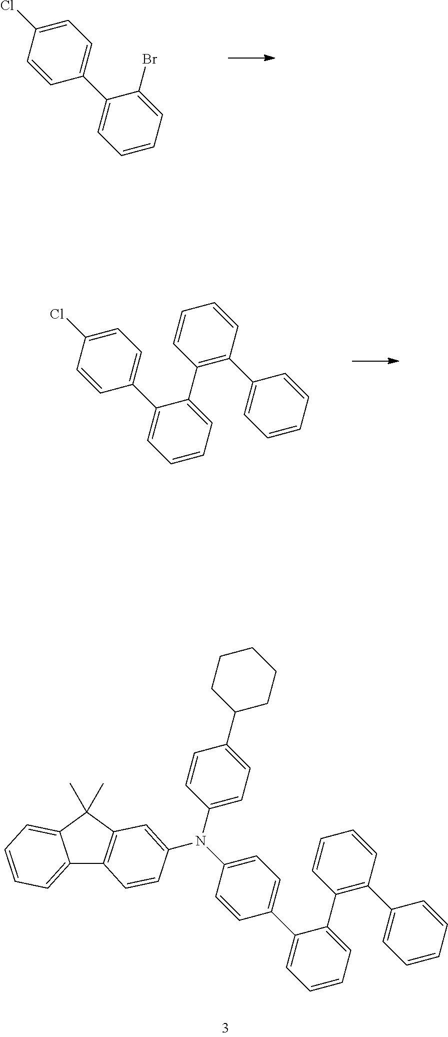 [SMILES: Clc1ccc(-c2ccccc2Br)cc1 ; Clc1ccc(-c2ccccc2-c2ccccc2-c2ccccc2)cc1 ; CC1(C)c2ccccc2-c2ccc(N(c3ccc(-c4ccccc4-c4ccccc4-c4ccccc4)cc3)c3ccc(C4CCCCC4)cc3)cc21]

### Synthesis of Intermediate Compound 3-1

[1,1′-biphenyl]-2-ylboronic acid (1.2 eq.), tetrakis(triphenylphosphine)palladium (0.05 eq.), and potassium carbonate (2.0 eq.) in 2-bromo-4′-chloro-1,1′-biphenyl (1.0 eq.) were dissolved in a solvent having a volumetric ratio of dioxane:H₂O of 4:1, followed by stirring under nitrogen atmosphere at a temperature of 110° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 3-1 was obtained (yield: 65%).

### Synthesis of Compound 3

Intermediate Compound 1-1 (1.0 eq.), Intermediate Compound 3-1 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 3 was obtained (yield: 63%). By FAB-MS, mass number m/z=671.36 was observed as a molecular ion peak. Thus, Compound 3 was identified.

### Synthesis Example 4 (Synthesis of Compound 5)

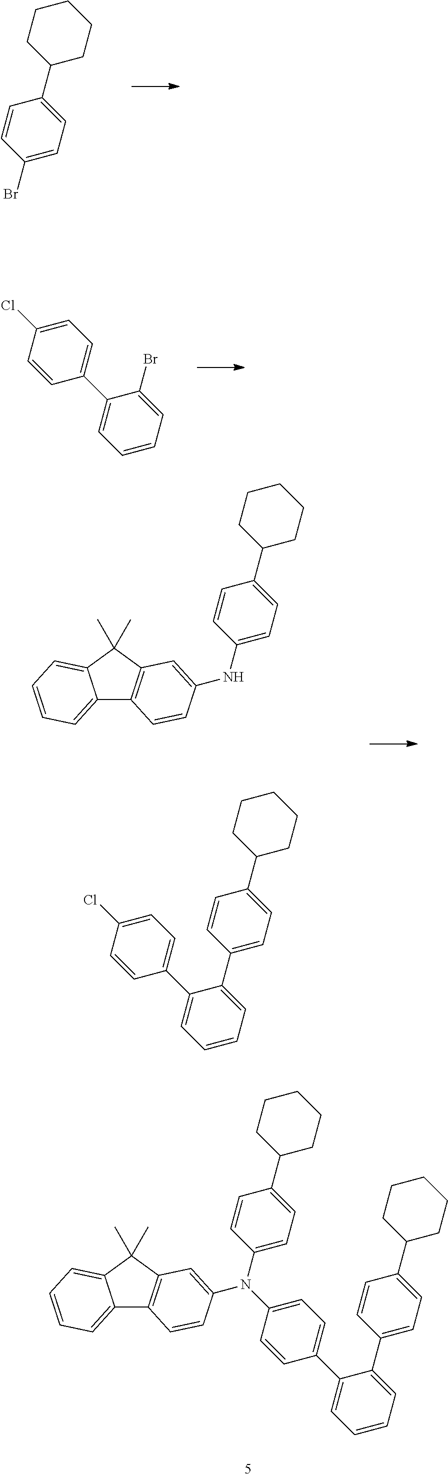 [SMILES: Brc1ccc(C2CCCCC2)cc1 ; Clc1ccc(-c2ccccc2Br)cc1 ; CC1(C)c2ccccc2-c2ccc(Nc3ccc(C4CCCCC4)cc3)cc21 ; Clc1ccc(-c2ccccc2-c2ccc(C3CCCCC3)cc2)cc1 ; CC1(C)c2ccccc2-c2ccc(N(c3ccc(-c4ccccc4-c4ccc(C5CCCCC5)cc4)cc3)c3ccc(C4CCCCC4)cc3)cc21]

### Synthesis of Intermediate Compound 5-1

1-Bromo-4-cyclohexylbenzene (1.0 eq.), 9,9-dimethyl-9H-fluoren-2-amine (1.2 eq.), tris(dibenzylideneacetone)dipalladium(0) (0.05 eq.), 2-dicyclohexylphosphino-2′,6′-dimethoxybiphenyl (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 5-1 was obtained (yield: 68%).

### Synthesis of Intermediate Compound 5-2

2-bromo-4′-chloro-1,1′-biphenyl (1.0 eq.), (4-cyclohexylphenyl)boronic acid (1.2 eq.), tetrakis(triphenylphosphine)palladium (0.05 eq.), and potassium carbonate (6.0 eq.) were dissolved in a solvent having a volumetric ratio of THF:H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 5-2 was obtained (yield: 65).

### Synthesis of Compound 5

Intermediate Compound 5-1 (1.0 eq.), Intermediate Compound 5-2 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 5 was obtained (yield: 65%). By fast-atom bombardment mass spectrometry (FAB-MS), mass number m/z=677.40 was observed as a molecular ion peak. Thus, Compound 5 was identified.

### Synthesis Example 5 (Synthesis of Compound 23)

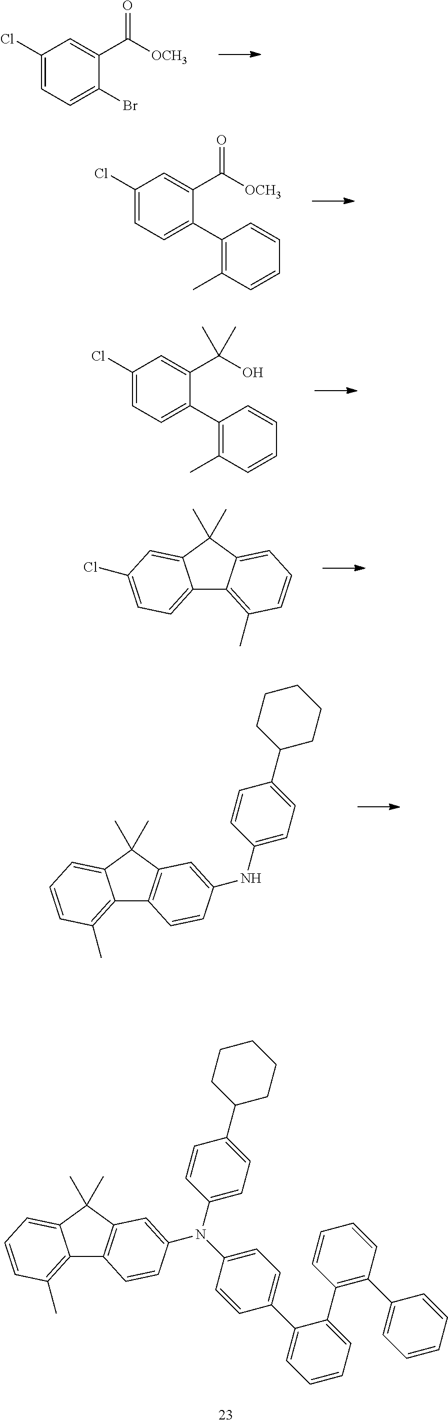

### Synthesis of Intermediate Compound 23-1

2-methylphenylboronic acid (1.2 eq.), tetrakis(triphenylphosphine)palladium (0.05 eq.), and potassium carbonate (2.0 eq.) in methyl 2-bromo-5-chlorobenzoate (1.0 eq.) were dissolved in a solvent having a volumetric ratio of THF:H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 23-1 was obtained (yield: 85%).

### Synthesis of Intermediate Compound 23-2

200 mL of THF was added to Intermediate Compound 23-1 (1.0 eq.), followed by cooling to 0° C. Under nitrogen atmosphere, methyl magnesium bromide (4.0 eq.) was added dropwise thereto, followed by stirring for 1 hour. Ammonium chloride aqueous solution was added dropwise thereto for neutralization, extraction was performed with ethyl acetate and water three times (e.g., dissolved in ethyl acetate and washed with water three times), and the resulting organic layer was dried utilizing MgSO₄ and then dried under reduced pressure. Through column chromatography, Intermediate Compound 23-2 was obtained (yield: 78%).

### Synthesis of Intermediate Compound 23-3

Trifluoromethanesulfonic acid (10 eq.) was slowly added dropwise to Intermediate Compound 23-2 (1.0 eq.) and dichloromethane (DCM) (200 mL), followed by stirring at a temperature of 50° C. Once the mixture was cooled to room temperature and extracted three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 23-3 was obtained (yield: 72%).

### Synthesis of Intermediate Compound 23-4

4-cyclohexylaniline (1.0 eq.), Intermediate Compound 23-3 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 80° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 23-4 was obtained (yield: 85%).

### Synthesis of Compound 23

Intermediate Compound 23-4 (1.0 eq.), Intermediate Compound 3-1 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 23 was obtained (yield: 63%). By FAB-MS, mass number m/z=685.37 was observed as a molecular ion peak. Thus, Compound 23 was identified.

### Synthesis Example 6 (Synthesis of Compound 83)

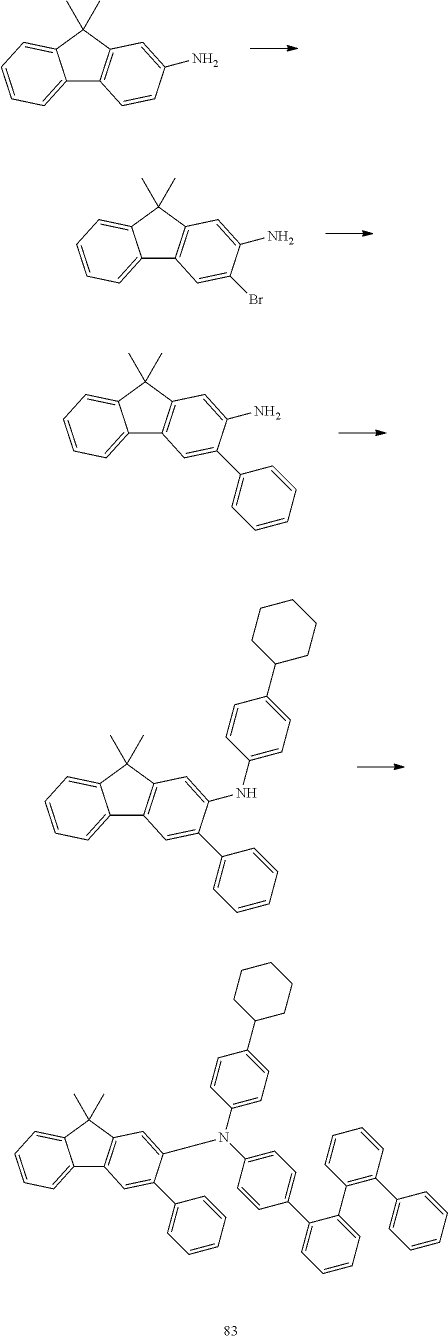 [SMILES: CC1(C)c2ccccc2-c2ccc(N)cc21 ; CC1(C)c2ccccc2-c2cc(Br)c(N)cc21 ; CC1(C)c2ccccc2-c2cc(-c3ccccc3)c(N)cc21 ; CC1(C)c2ccccc2-c2cc(-c3ccccc3)c(Nc3ccc(C4CCCCC4)cc3)cc21 ; CC1(C)c2ccccc2-c2cc(-c3ccccc3)c(N(c3ccc(-c4ccccc4-c4ccccc4-c4ccccc4)cc3)c3ccc(C4CCCCC4)cc3)cc21]

### Synthesis of Intermediate Compound 83-1

9,9-dimethyl-9H-fluoren-2-amine (1.0 eq.) was dissolved in DCM, and N-bromosuccinimide (1.0 eq.) was slowly added dropwise thereto at a temperature of 0° C. Once the mixture was warmed to room temperature and washed three times utilizing ethyl acetate and water in which sodium thiosulfate was dissolved, the resulting organic layer was dried utilizing MgSO₄ and then dried under reduced pressure. Through column chromatography, Intermediate Compound 83-1 was obtained (yield: 95%).

### Synthesis of Intermediate Compound 83-2

Intermediate Compound 83-1 (1.0 eq.), tetrakis(triphenylphosphine)palladium (0.05 eq.), and potassium carbonate (2.0 eq.) in phenylboronic acid (1.2 eq.) were dissolved in a solvent having a volumetric ratio of THF:H₂O of 4:1, followed by stirring under nitrogen atmosphere at a temperature of 80° C. for 2 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 83-2 was obtained (yield: 85%).

### Synthesis of Intermediate Compound 83-3

Intermediate Compound 83-2 (1.0 eq.), 1-bromo-4-cyclohexylbenzene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 80° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 83-3 was obtained (yield: 85%).

### Synthesis of Compound 83

Intermediate Compound 83-3 (1.0 eq.), Intermediate Compound 3-1 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 83 was obtained (yield: 63%). By FAB-MS, mass number m/z=747.39 was observed as a molecular ion peak. Thus, Compound 83 was identified.

### Synthesis Example 7 (Synthesis of Compound 103)

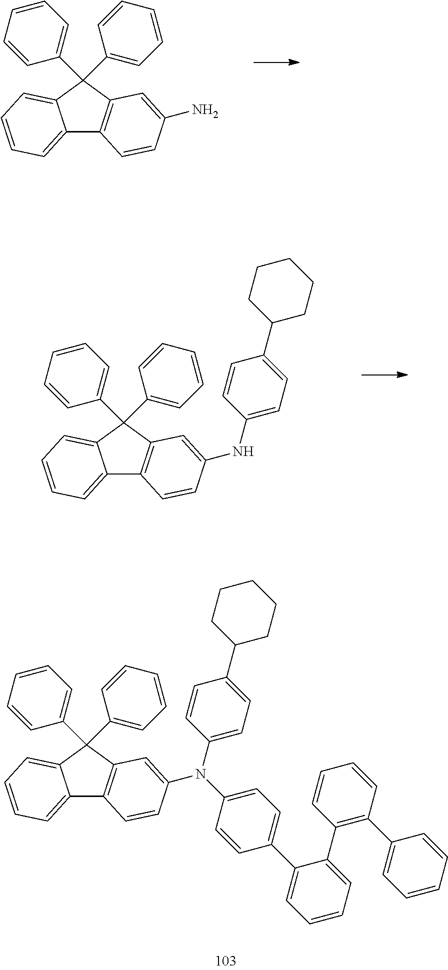 [SMILES: Nc1ccc2c(c1)C(c1ccccc1)(c1ccccc1)c1ccccc1-2 ; c1ccc(C2(c3ccccc3)c3ccccc3-c3ccc(Nc4ccc(C5CCCCC5)cc4)cc32)cc1 ; c1ccc(-c2ccccc2-c2ccccc2-c2ccc(N(c3ccc(C4CCCCC4)cc3)c3ccc4c(c3)C(c3ccccc3)(c3ccccc3)c3ccccc3-4)cc2)cc1]

### Synthesis of Intermediate Compound 103-1

9,9-diphenyl-9H-fluoren-2-amine (1.0 eq.), 1-bromo-4-cyclohexylbenzene (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 80° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 103-1 was obtained (yield: 87%).

### Synthesis of Compound 103

Intermediate Compound 103-1 (1.0 eq.), Intermediate Compound 3-1 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 103 was obtained (yield: 63%). By FAB-MS, mass number m/z=795.39 was observed as a molecular ion peak. Thus, Compound 103 was identified.

### Synthesis Example 8: Synthesis of Compound 123

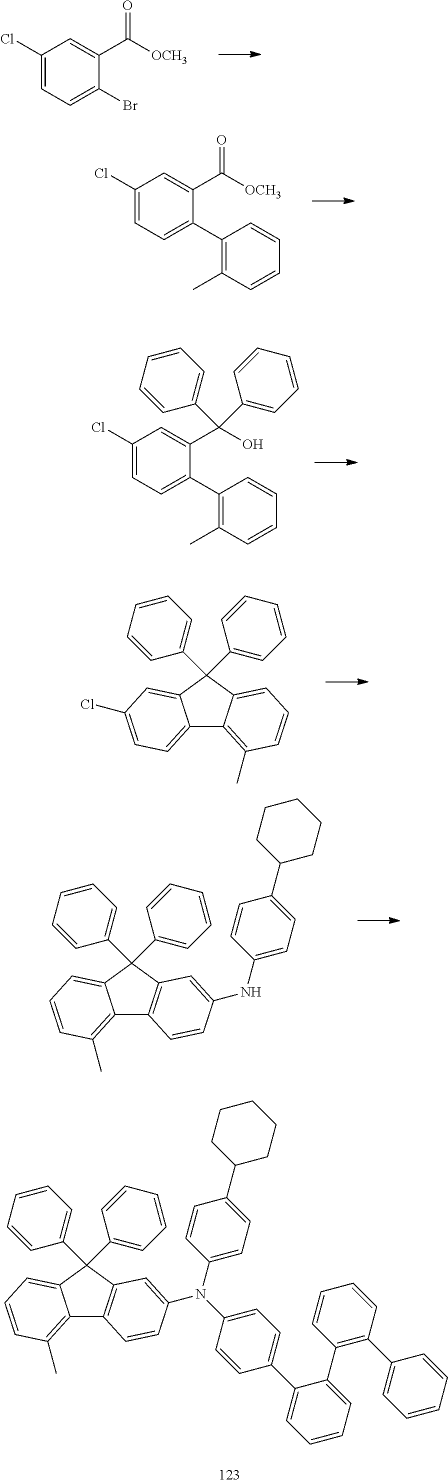

### Synthesis of Intermediate Compound 123-1

2-methylphenylboronic acid (1.2 eq.), tetrakis(triphenylphosphine)palladium (0.05 eq.), and potassium carbonate (2.0 eq.) in methyl 2-bromo-5-chlorobenzoate (1.0 eq.) were dissolved in a solvent having a volumetric ratio of THF:H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 123-1 was obtained (yield: 85%).

### Synthesis of Intermediate Compound 123-2

200 mL of THF was added to Intermediate Compound 123-1 (1.0 eq.), followed by cooling to 0° C. Under nitrogen atmosphere, phenyl magnesium bromide (4.0 eq.) was added dropwise thereto, followed by stirring for 1 hour. Ammonium chloride aqueous solution was added dropwise thereto for neutralization, extraction was performed with ethyl acetate and water three times (e.g., dissolved in ethyl acetate and washed with water three times), and the resulting organic layer was dried utilizing MgSO₄ and then dried under reduced pressure. Through column chromatography, Intermediate Compound 123-2 was obtained (yield: 76%).

### Synthesis of Intermediate Compound 123-3

Trifluoromethanesulfonic acid (10 eq.) was slowly added dropwise to Intermediate Compound 123-2 (1.0 eq.) and DCM (200 mL), followed by stirring at a temperature of 50° C. Once the mixture was cooled to room temperature and extracted three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 123-3 was obtained (yield: 68%).

### Synthesis of Intermediate Compound 123-4

4-cyclohexylaniline (1.0 eq.), Intermediate Compound 123-3 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 80° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 123-4 was obtained (yield: 85%).

### Synthesis of Compound 123

Intermediate Compound 123-4 (1.0 eq.), Intermediate Compound 3-1 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 123 was obtained (yield: 63%). By FAB-MS, mass number m/z=809.40 was observed as a molecular ion peak. Thus, Compound 123 was identified.

### Synthesis Example 9: (Synthesis of Compound 241)

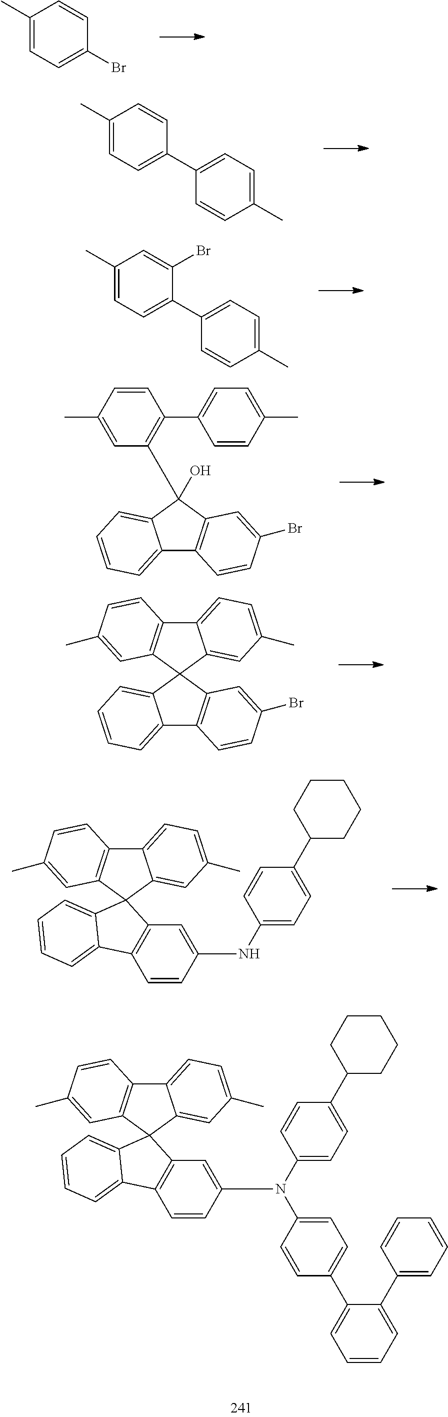

### Synthesis of Intermediate Compound 241-1

p-tolylboronic acid (1.2 eq.), tetrakis(triphenylphosphine)palladium (0.05 eq.), and potassium carbonate (2.0 eq.) in 1-bromo-4-methylbenzene (1.0 eq.) were dissolved in a solvent having a volumetric ratio of THF:H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 241-1 was obtained (yield: 85%).

### Synthesis of Intermediate Compound 241-2

Intermediate Compound 241-1 (1.0 eq.) was dissolved in DCM, and Br₂ (1.0 eq.) was slowly added thereto under a nitrogen atmosphere and at a temperature of 0° C. After stirring for 4 hours at room temperature, an organic layer resulting from washing three times with ethyl acetate and water was washed with sodium thiosulfate aqueous solution and dried utilizing MgSO₄ and then dried under reduced pressure. Through column chromatography, Intermediate Compound 241-2 was obtained (yield: 85%).

### Synthesis of Intermediate Compound 241-3

THF was added to Intermediate Compound 241-2 (1.0 eq.), followed by cooling to −78° C. Under nitrogen atmosphere, n-butyl lithium (1.1 eq.) was added dropwise thereto, followed by stirring for 1 hour at room temperature. 2-bromo-9H-fluoren-9-one (1.1 eq.) dissolved in THF was added dropwise thereto at room temperature, followed by stirring for 4 hours. Then, the resulting organic layer washed three times with ethyl acetate and water was dried utilizing MgSO₄ and then dried under reduced pressure. Through column chromatography, Intermediate Compound 241-3 was obtained (yield: 67%).

### Synthesis of Intermediate Compound 241-4

Trifluoromethanesulfonic acid (10 eq.) was slowly added dropwise to Intermediate Compound 241-3 (1.0 eq.) and DCM, followed by stirring at a temperature of 50° C. Once the mixture was cooled to room temperature and extracted three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 241-4 was obtained (yield: 72%).

### Synthesis of Intermediate Compound 241-5

4-cyclohexylaniline (1.0 eq.), Intermediate Compound 241-4 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 80° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 241-5 was obtained (yield: 85%).

### Synthesis of Compound 241

Intermediate Compound 241-5 (1.0 eq.), Intermediate Compound 1-2 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 241 was obtained (yield: 63%). By FAB-MS, mass number m/z=75.37 was observed as a molecular ion peak. Thus, Compound 241 was identified.

### Synthesis Example 10 (Synthesis of Compound 265)

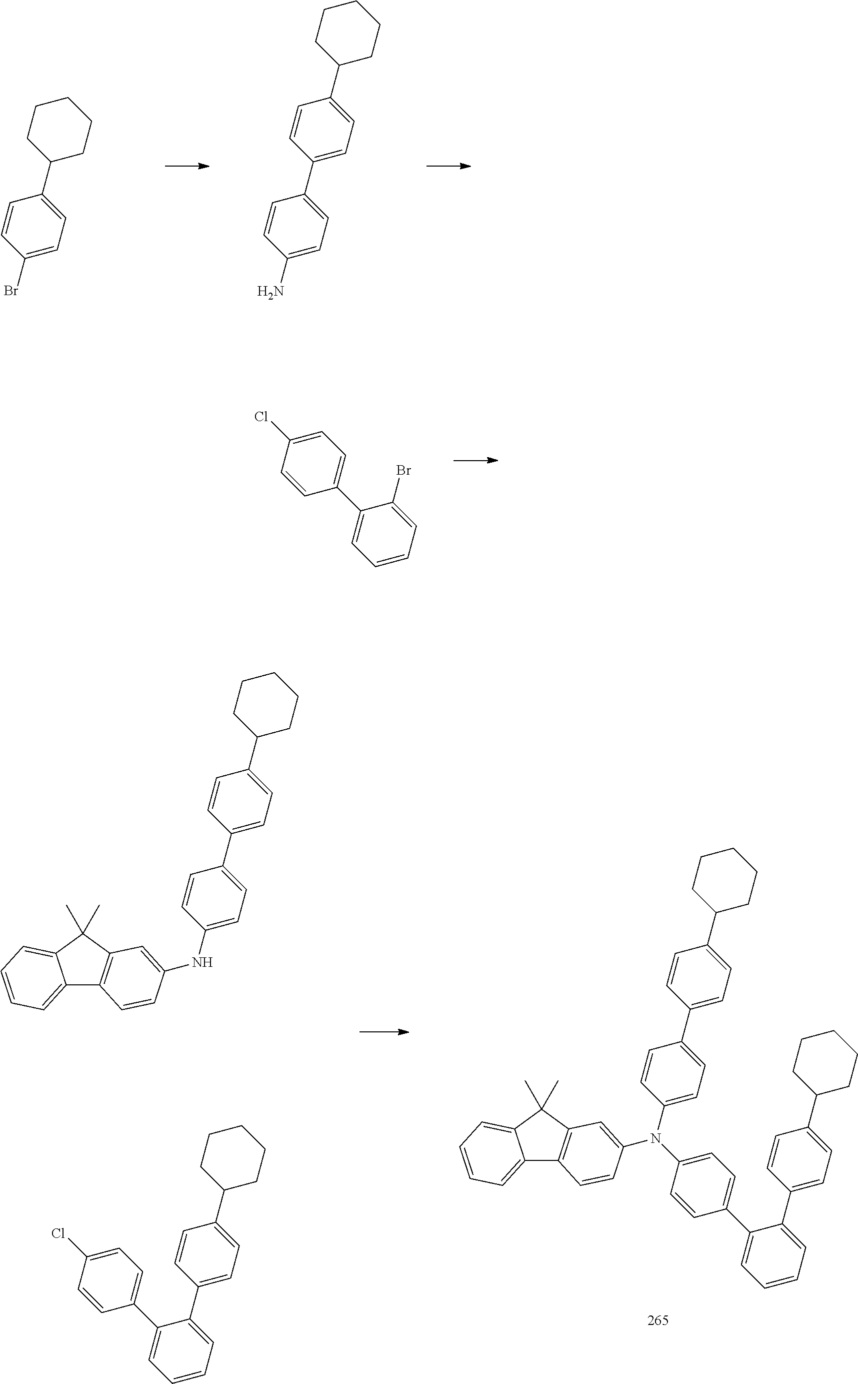 [SMILES: Brc1ccc(C2CCCCC2)cc1 ; Nc1ccc(-c2ccc(C3CCCCC3)cc2)cc1 ; Clc1ccc(-c2ccccc2Br)cc1 ; CC1(C)c2ccccc2-c2ccc(Nc3ccc(-c4ccc(C5CCCCC5)cc4)cc3)cc21 ; Clc1ccc(-c2ccccc2-c2ccc(C3CCCCC3)cc2)cc1 ; CC1(C)c2ccccc2-c2ccc(N(c3ccc(-c4ccc(C5CCCCC5)cc4)cc3)c3ccc(-c4ccccc4-c4ccc(C5CCCCC5)cc4)cc3)cc21]

### Synthesis of Intermediate Compound 265-1

1-bromo-4-cyclohexylbenzene (1.0 eq.), (4-aminophenyl)boronic acid (1.0 eq.), tetrakis(triphenylphosphine)palladium (0.05 eq.), and potassium carbonate (6.0 eq.) were dissolved in a solvent having a volumetric ratio of THF:H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 265-1 was obtained (yield: 65%).

### Synthesis of Intermediate Compound 265-2

Intermediate Compound 265-1 (1.0 eq.), 2-bromo-9,9-dimethyl-9H-fluorene (1.2 eq.), tris(dibenzylideneacetone)dipalladium(0) (0.05 eq.), 2-dicyclohexylphosphino-1 2′,6′-dimethoxybiphenyl (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 265-2 was obtained (yield: 68%).

### Synthesis of Intermediate Compound 265-3

2-bromo-4′-chloro-1,1′-biphenyl (1.0 eq.), (4-cyclohexylphenyl)boronic acid (1.2 eq.), tetrakis(triphenylphosphine)palladium (0.05 eq.), and potassium carbonate (6.0 eq.) were dissolved in a solvent having a volumetric ratio of THF:H₂O of 4:1, followed by stirring under nitrogen atmosphere at a temperature of 80° C. for 12 hours. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 265-3 was obtained (yield: 65%).

### Synthesis of Compound 265

Intermediate Compound 265-2 (1.0 eq.), Intermediate Compound 265-3 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 265 was obtained (yield: 65%). By FAB-MS, mass number m/z=753.43 was observed as a molecular ion peak. Thus, Compound 265 was identified.

### Synthesis Example 11 (Synthesis of Compound 268)

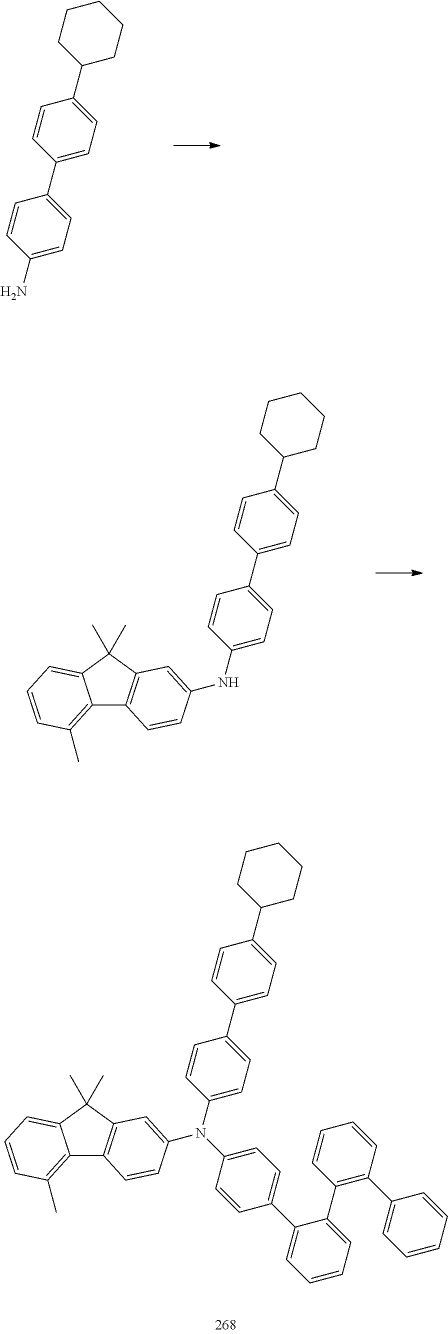 [SMILES: Nc1ccc(-c2ccc(C3CCCCC3)cc2)cc1 ; Cc1cccc2c1-c1ccc(Nc3ccc(-c4ccc(C5CCCCC5)cc4)cc3)cc1C2(C)C ; Cc1cccc2c1-c1ccc(N(c3ccc(-c4ccc(C5CCCCC5)cc4)cc3)c3ccc(-c4ccccc4-c4ccccc4-c4ccccc4)cc3)cc1C2(C)C]

### Synthesis of Intermediate Compound 268-1

Intermediate Compound 265-1 (1.0 eq.), Intermediate Compound 23-3 (1.2 eq.), tris(dibenzylideneacetone)dipalladium(0) (0.05 eq.), 2-dicyclohexylphosphino-2′,6′-dimethoxybiphenyl (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 268-1 was obtained (yield: 68%).

### Synthesis of Compound 268

Intermediate Compound 268-1 (1.0 eq.), Intermediate Compound 3-1 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 110° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 268 was obtained (yield: 80%). By FAB-MS, mass number m/z=761.40 was observed as a molecular ion peak. Thus, Compound 268 was identified.

### Synthesis Example 12 (Synthesis of Compound 270)

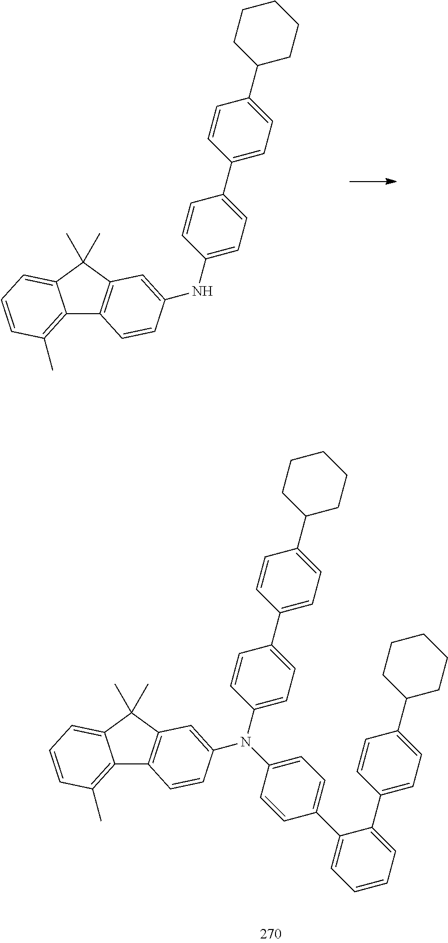 [SMILES: Cc1cccc2c1-c1ccc(Nc3ccc(-c4ccc(C5CCCCC5)cc4)cc3)cc1C2(C)C ; Cc1cccc2c1-c1ccc(N(c3ccc(-c4ccc(C5CCCCC5)cc4)cc3)c3ccc(-c4ccccc4-c4ccc(C5CCCCC5)cc4)cc3)cc1C2(C)C]

### Synthesis of Compound 270

Intermediate Compound 268-1 (1.0 eq.), Intermediate Compound 5-2 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 110° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 270 was obtained (yield: 80%). By FAB-MS, mass number m/z=767.45 was observed as a molecular ion peak. Thus, Compound 270 was identified.

### Synthesis Example 13 (Synthesis of Compound 291)

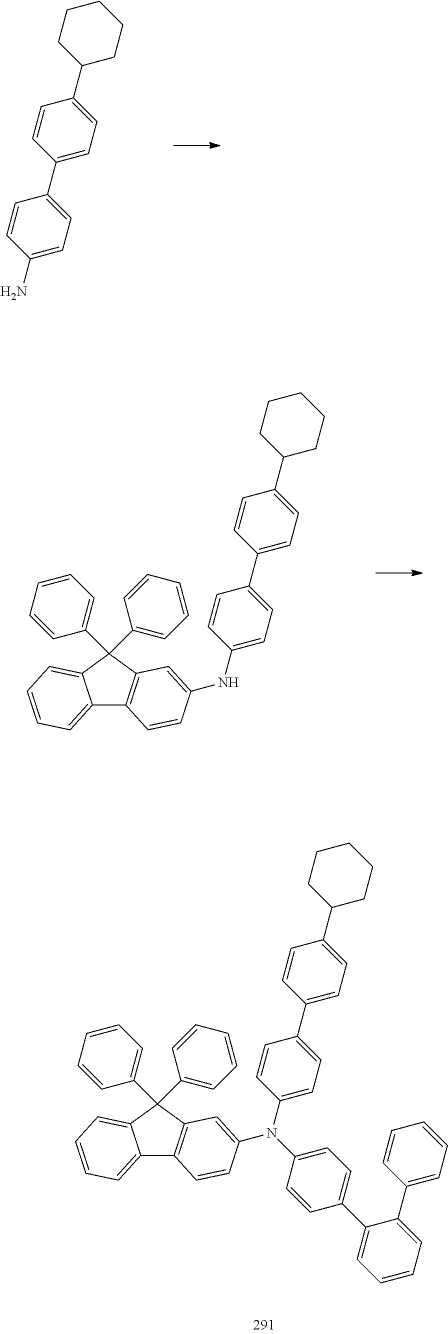 [SMILES: Nc1ccc(-c2ccc(C3CCCCC3)cc2)cc1 ; c1ccc(C2(c3ccccc3)c3ccccc3-c3ccc(Nc4ccc(-c5ccc(C6CCCCC6)cc5)cc4)cc32)cc1 ; c1ccc(-c2ccccc2-c2ccc(N(c3ccc(-c4ccc(C5CCCCC5)cc4)cc3)c3ccc4c(c3)C(c3ccccc3)(c3ccccc3)c3ccccc3-4)cc2)cc1]

### Synthesis of Intermediate Compound 291-1

Intermediate Compound 265-1 (1.0 eq.), 2-bromo-9,9-diphenyl-9H-fluorene (1.2 eq.), tris(dibenzylideneacetone)dipalladium(0) (0.05 eq.), 2-dicyclohexylphosphino-2′,6′-dimethoxybiphenyl (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 291-1 was obtained (yield: 68%).

### Synthesis of Compound 291

Intermediate Compound 291-1 (1.0 eq.), Intermediate Compound 1-2 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 110° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Compound 291 was obtained through column chromatography (yield: 80%). By FAB-MS, mass number m/z=795.39 was observed as a molecular ion peak. Thus, Compound 291 was identified.

### Synthesis Example 14 (Synthesis of Compound 296)

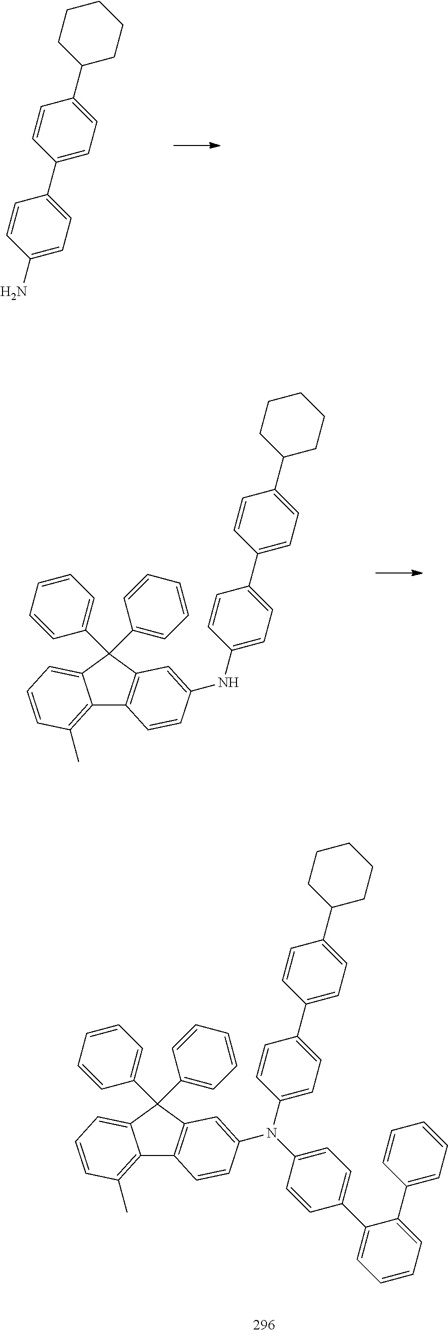 [SMILES: Nc1ccc(-c2ccc(C3CCCCC3)cc2)cc1 ; Cc1cccc2c1-c1ccc(Nc3ccc(-c4ccc(C5CCCCC5)cc4)cc3)cc1C2(c1ccccc1)c1ccccc1 ; Cc1cccc2c1-c1ccc(N(c3ccc(-c4ccc(C5CCCCC5)cc4)cc3)c3ccc(-c4ccccc4-c4ccccc4)cc3)cc1C2(c1ccccc1)c1ccccc1]

### Synthesis of Intermediate Compound 296-1

Intermediate Compound 265-1 (1.0 eq.), Intermediate Compound 123-3 (1.2 eq.), tris(dibenzylideneacetone)dipalladium(0) (0.05 eq.), 2-dicyclohexylphosphino-2′,6′-dimethoxybiphenyl (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 296-1 was obtained (yield: 68%).

### Synthesis of Compound 296

Intermediate Compound 296-1 (1.0 eq.), Intermediate Compound 1-2 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 110° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Finally, through column chromatography, Compound 268 was obtained (yield: 80%). By FAB-MS, mass number m/z=809.40 was observed as a molecular ion peak. Thus, Compound 296 was identified.

### Synthesis Example 15 (Synthesis of Compound 356)

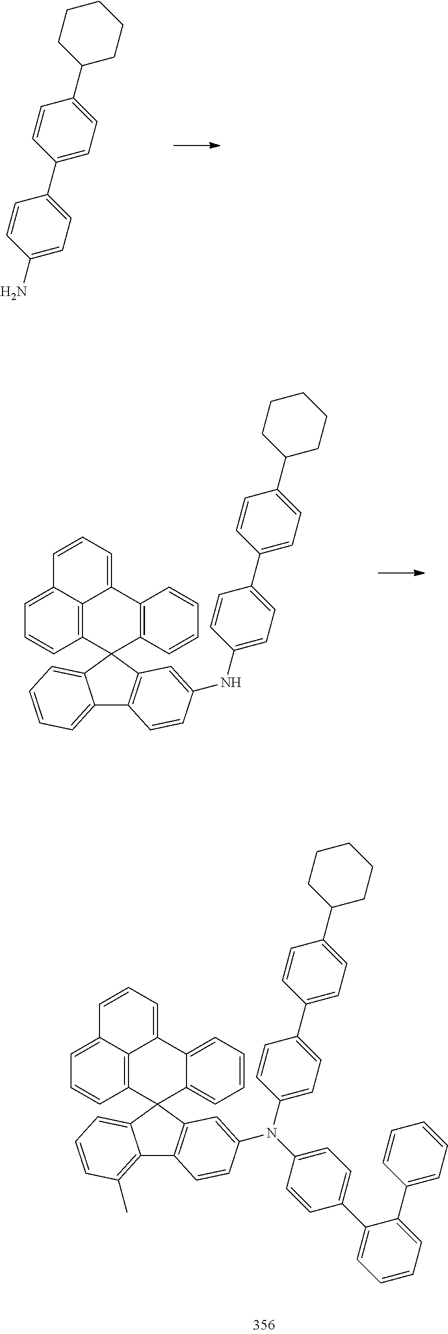 [SMILES: Nc1ccc(-c2ccc(C3CCCCC3)cc2)cc1 ; c1ccc2c(c1)-c1ccc(Nc3ccc(-c4ccc(C5CCCCC5)cc4)cc3)cc1C21c2ccccc2-c2cccc3cccc1c23 ; Cc1cccc2c1-c1ccc(N(c3ccc(-c4ccc(C5CCCCC5)cc4)cc3)c3ccc(-c4ccccc4-c4ccccc4)cc3)cc1C21c2ccccc2-c2cccc3cccc1c23]

### Synthesis of Intermediate Compound 356-1

Intermediate Compound 265-1 (1.0 eq.), 2′-bromospiro[benzo[de]anthracene-7,9′-fluorene] (1.1 eq.), tris(dibenzylideneacetone)dipalladium(0) (0.05 eq.), 2-dicyclohexylphosphino-2′,6′-dimethoxybiphenyl (0.10 eq.), and sodium tert-butoxide (2.0 eq.) were dissolved in toluene, followed by stirring under nitrogen atmosphere at a temperature of 100° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Through column chromatography, Intermediate Compound 356-1 was obtained (yield: 68%).

### Synthesis of Compound 356

Intermediate Compound 356-1 (1.0 eq.), Intermediate Compound 1-2 (1.2 eq.), tris(dibenzylideneacetone)dipalladium(0) (0.05 eq.), tri-tert-butylphosphine (0.10 eq.), and sodium tert-butoxide (2.0 eq.) were dissolved in toluene, followed by stirring under nitrogen atmosphere at a temperature of 110° C. for 1 hour. Once the mixture was cooled and washed three times utilizing ethyl acetate and water (e.g., dissolved in ethyl acetate and washed with water three times), the resulting organic layer was dried utilizing MgSO₄ and then dried under reduced pressure. Compound 356 was obtained through column chromatography (yield: 80%). By FAB-MS, mass number m/z=843.39 was observed as a molecular ion peak. Thus, Compound 356 was identified.

Methods of synthesizing compounds other than the compounds synthesized in Synthesis Examples 1 to 15 may be easily understood to those skilled in the art by referring to the synthesis pathways and raw materials described above.

### Evaluation Example 1

The lowest unoccupied molecular orbital (LUMO) energy level, highest occupied molecular orbital (HOMO) energy level, Bandgap, maximum emission wavelength (λₘₐₓ), and refractive index of each of the Compounds prepared in the Synthesis Examples were measured as shown in Table 1. The results thereof are shown in Table 2.

TABLE 1

| | |
|---|---|
| HOMO | A potential (V)-current (A) graph of each compound was |
| energy level | obtained by utilizing cyclic voltammetry (CV) (electrolyte: |
| evaluation | 0.1M BBu₄NPF₆/solvent: dimethyl formamide (DMF)/ |
| method | electrode: 3 electrode system (working electrode: glassy |
| | carbon (GC), reference electrode: Ag/AgCl, auxiliary |
| | electrode: Pt)), and then, from oxidation onset of the graph, |
| | a HOMO energy level of the compound was calculated. |
| LUMO | A potential (V)-current (A) graph of each compound was |
| energy level | obtained by utilizing cyclic voltammetry (CV) (electrolyte: |
| evaluation | 0.1M BBu₄NPF₆/solvent: dimethyl formamide (DMF)/ |
| method | electrode: 3 electrode system (working electrode: glassy |
| | carbon (GC), reference electrode: Ag/AgCl, auxiliary |
| | electrode: Pt)), and then, from reduction onset of the graph, |
| | a LUMO energy level of the compound was calculated. |
| Refractive | A refractive index measurement material was vacuum- |
| index | deposited on a bare glass at a deposition rate of 1 Å/s |
| | utilizing a vacuum evaporator to produce a single film of a |
| | measurement material. After measuring the refractive index |
| | utilizing ellipsometry, the refractive index of the desired or |
| | suitable wavelength after modeling fitting work was |
| | measured. |

TABLE 2

| | | | | | Refrac− |
|---|---|---|---|---|---|
| | | | | | tive |
| | | | | Bandgap | index |
| Compound | | HOMO (eV) | LUMO (eV) | (eV) | (η, 450 nm) |
| Synthesis | Compound | −5.15 | −2.20 | 2.95 | 1.79 |
| Example | 1 | | | | |
| 1 | | | | | |
| Synthesis | Compound | −5.14 | −2.20 | 2.94 | 1.76 |
| Example | 2 | | | | |
| 2 | | | | | |
| Synthesis | Compound | −5.15 | −2.20 | 2.95 | 1.79 |
| Example | 3 | | | | |
| 3 | | | | | |
| Synthesis | Compound | −5.14 | −2.19 | 2.95 | 1.77 |
| Example | 5 | | | | |
| 4 | | | | | |
| Synthesis | Compound | −5.15 | −2.20 | 2.95 | 1.80 |
| Example | 23 | | | | |
| 5 | | | | | |
| Synthesis | Compound | −5.16 | −2.15 | 3.01 | 1.79 |
| Example | 83 | | | | |
| 6 | | | | | |
| Synthesis | Compound | −5.19 | −2.18 | 3.01 | 1.80 |
| Example | 103 | | | | |
| 7 | | | | | |
| Synthesis | Compound | −5.19 | −1.91 | 3.28 | 1.81 |
| Example | 123 | | | | |
| 8 | | | | | |
| Synthesis | Compound | −5.19 | −2.19 | 3.00 | 1.82 |
| Example | 241 | | | | |
| 9 | | | | | |
| Synthesis | Compound | −5.16 | −2.20 | 2.96 | 1.80 |
| Example | 265 | | | | |
| 10 | | | | | |
| Synthesis | Compound | −5.16 | −2.20 | 2.96 | 1.80 |
| Example | 268 | | | | |
| 11 | | | | | |
| Synthesis | Compound | −5.15 | −2.20 | 2.95 | 1.79 |
| Example | 270 | | | | |
| 12 | | | | | |
| Synthesis | Compound | −5.20 | −1.91 | 3.29 | 1.80 |
| Example | 291 | | | | |
| 13 | | | | | |
| Synthesis | Compound | −5.20 | −1.91 | 3.29 | 1.81 |
| Example | 296 | | | | |
| 14 | | | | | |
| Synthesis | Compound | −5.20 | −1.91 | 3.29 | 1.82 |
| Example | 356 | | | | |
| 15 | | | | | |

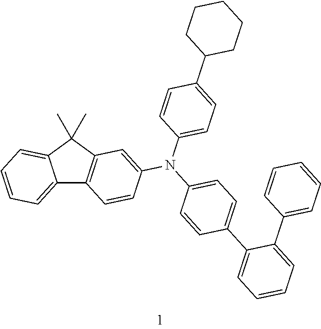 [SMILES: CC1(C)c2ccccc2-c2ccc(N(c3ccc(-c4ccccc4-c4ccccc4)cc3)c3ccc(C4CCCCC4)cc3)cc21]

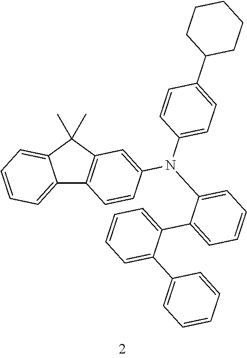 [SMILES: CC1(C)c2ccccc2-c2ccc(N(c3ccc(C4CCCCC4)cc3)c3ccccc3-c3ccccc3-c3ccccc3)cc21]

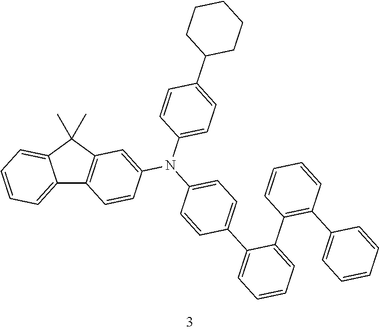 [SMILES: CC1(C)c2ccccc2-c2ccc(N(c3ccc(-c4ccccc4-c4ccccc4-c4ccccc4)cc3)c3ccc(C4CCCCC4)cc3)cc21]

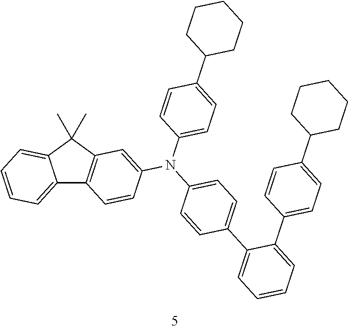 [SMILES: CC1(C)c2ccccc2-c2ccc(N(c3ccc(-c4ccccc4-c4ccc(C5CCCCC5)cc4)cc3)c3ccc(C4CCCCC4)cc3)cc21]

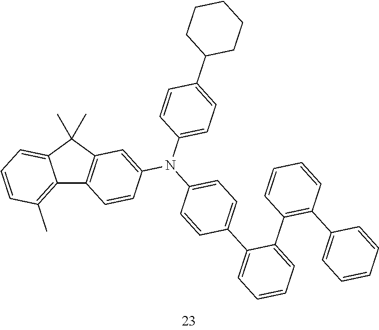 [SMILES: Cc1cccc2c1-c1ccc(N(c3ccc(-c4ccccc4-c4ccccc4-c4ccccc4)cc3)c3ccc(C4CCCCC4)cc3)cc1C2(C)C]

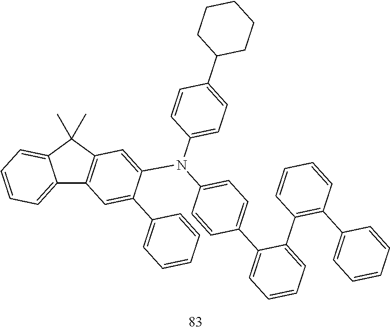 [SMILES: CC1(C)c2ccccc2-c2cc(-c3ccccc3)c(N(c3ccc(-c4ccccc4-c4ccccc4-c4ccccc4)cc3)c3ccc(C4CCCCC4)cc3)cc21]

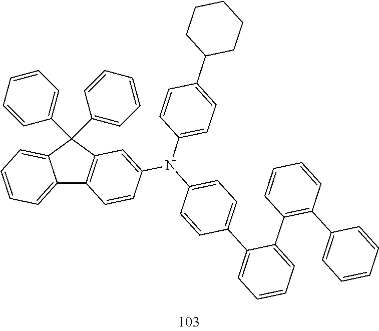 [SMILES: c1ccc(-c2ccccc2-c2ccccc2-c2ccc(N(c3ccc(C4CCCCC4)cc3)c3ccc4c(c3)C(c3ccccc3)(c3ccccc3)c3ccccc3-4)cc2)cc1]

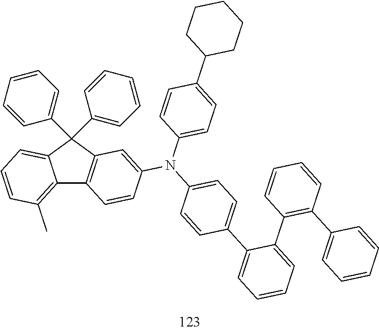 [SMILES: Cc1cccc2c1-c1ccc(N(c3ccc(-c4ccccc4-c4ccccc4-c4ccccc4)cc3)c3ccc(C4CCCCC4)cc3)cc1C2(c1ccccc1)c1ccccc1]

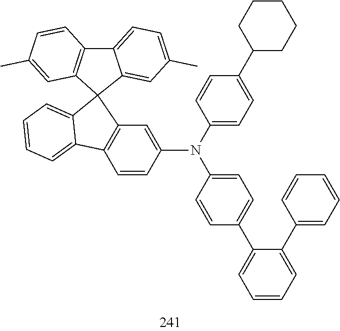 [SMILES: Cc1ccc2c(c1)C1(c3ccccc3-c3ccc(N(c4ccc(-c5ccccc5-c5ccccc5)cc4)c4ccc(C5CCCCC5)cc4)cc31)c1cc(C)ccc1-2]

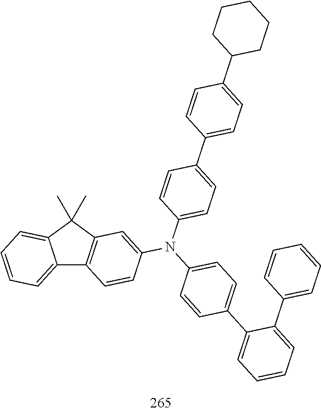 [SMILES: CC1(C)c2ccccc2-c2ccc(N(c3ccc(-c4ccc(C5CCCCC5)cc4)cc3)c3ccc(-c4ccccc4-c4ccccc4)cc3)cc21]

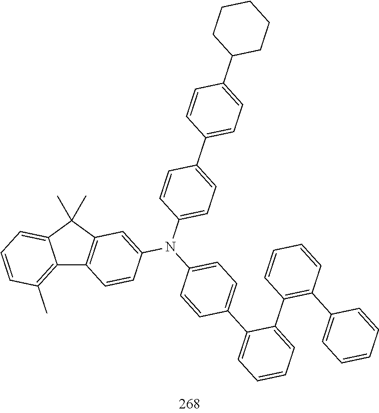 [SMILES: Cc1cccc2c1-c1ccc(N(c3ccc(-c4ccc(C5CCCCC5)cc4)cc3)c3ccc(-c4ccccc4-c4ccccc4-c4ccccc4)cc3)cc1C2(C)C]

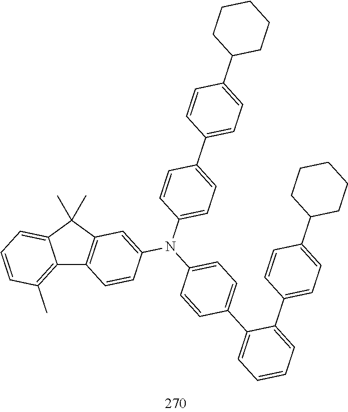 [SMILES: Cc1cccc2c1-c1ccc(N(c3ccc(-c4ccc(C5CCCCC5)cc4)cc3)c3ccc(-c4ccccc4-c4ccc(C5CCCCC5)cc4)cc3)cc1C2(C)C]

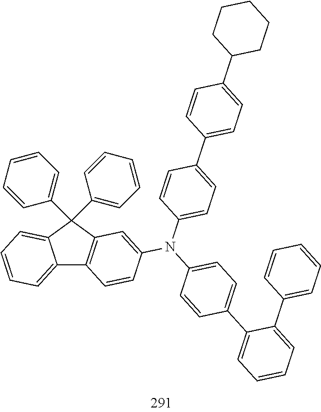 [SMILES: c1ccc(-c2ccccc2-c2ccc(N(c3ccc(-c4ccc(C5CCCCC5)cc4)cc3)c3ccc4c(c3)C(c3ccccc3)(c3ccccc3)c3ccccc3-4)cc2)cc1]

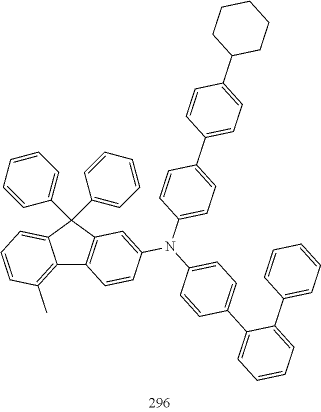 [SMILES: Cc1cccc2c1-c1ccc(N(c3ccc(-c4ccc(C5CCCCC5)cc4)cc3)c3ccc(-c4ccccc4-c4ccccc4)cc3)cc1C2(c1ccccc1)c1ccccc1]

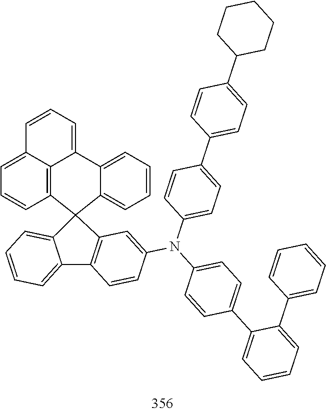 [SMILES: c1ccc(-c2ccccc2-c2ccc(N(c3ccc(-c4ccc(C5CCCCC5)cc4)cc3)c3ccc4c(c3)C3(c5ccccc5-4)c4ccccc4-c4cccc5cccc3c45)cc2)cc1]

### Example 1

As an anode, a 15 Ohms per square centimeter (0/cm²) (1,200 Å) ITO glass substrate (available from Corning Co., Ltd) was cut to a size of 50 millimeters (mm)×50 mm×0.7 mm, sonicated in isopropyl alcohol and pure water for 5 minutes in each solvent, cleaned with ultraviolet rays for 30 minutes, and then ozone, and was mounted on a vacuum deposition apparatus.

2-TNATA was vacuum-deposited on the anode to form a hole injection layer having a thickness of 600 Å. Compound 1 was then vacuum-deposited on the hole injection layer to form a hole transport layer having a thickness of 300 Å.

9,10-di(naphthalen-2-yl)anthracene (DNA) as a host material and 4,4′-bis[2-(4-(N,N-diphenylamino)phenyl)vinyl]biphenyl (DPAVBi) as a dopant material were co-deposited concurrently (e.g., simultaneously) on the hole transport layer at a weight ratio of 98:2 to form an emission layer having a thickness of 300 Å.

Then, Alq3 was vacuum-deposited on the emission layer to form an electron transport layer having a thickness of 300 Å, LiF was vacuum-deposited on the electron transport layer to form an electron injection layer having a thickness of 10 Å, and Al was vacuum-deposited on the electron injection layer to form a cathode having a thickness of 3,000 Å, thereby completing the manufacture of an organic light-emitting device.

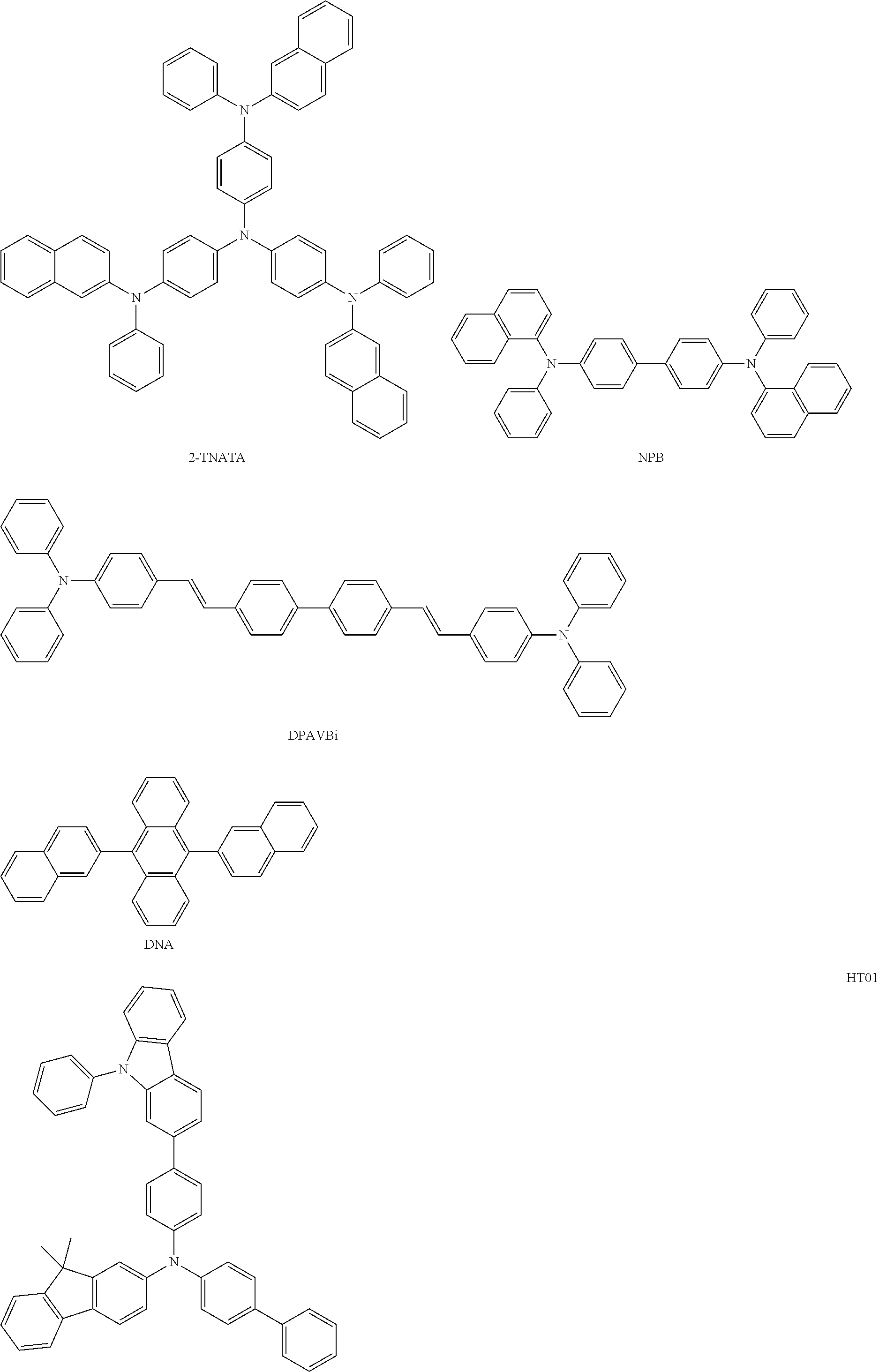

### Examples 2 to 15 and Comparative Examples 1 to 13

Organic light-emitting devices of Examples 2 to 15 and Comparative Examples 1 to 13 were each manufactured in substantially the same manner as in Example 1, except that Compounds 2, 3, 5, 23, 83, 103, 123, 241, 265, 268, 270, 291, 296, and 351 and Comparative Example Compounds 1 to 13 were respectively utilized instead of Compound 1 in forming a hole transport layer.

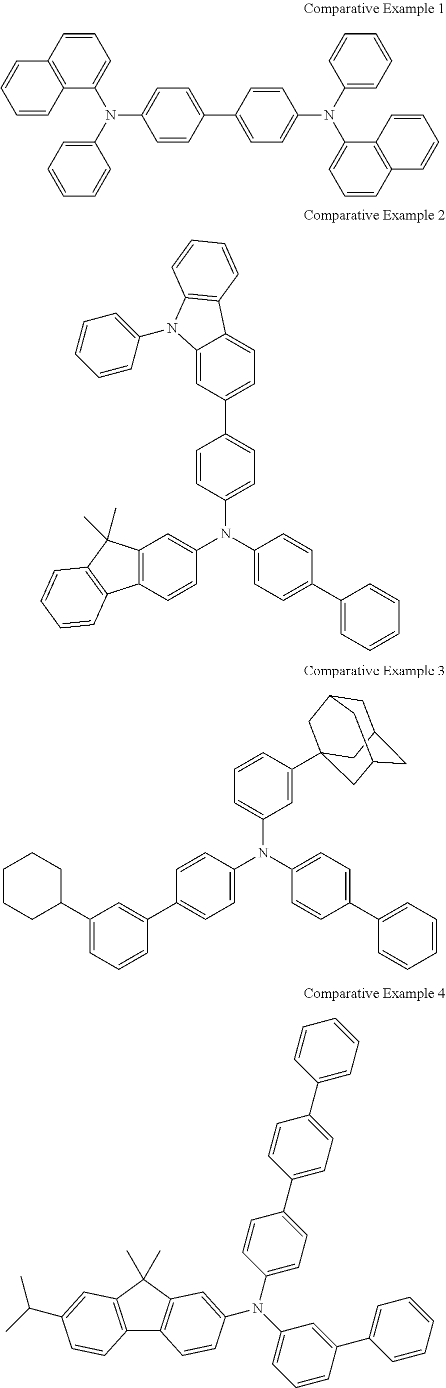 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccc(-c4ccccc4)cc3)c3ccc(-c4ccc5c6ccccc6n(-c6ccccc6)c5c4)cc3)cc21 ; c1ccc(-c2ccc(N(c3ccc(-c4cccc(C5CCCCC5)c4)cc3)c3cccc(C45CC6CC(CC(C6)C4)C5)c3)cc2)cc1 ; CC(C)c1ccc2c(c1)C(C)(C)c1cc(N(c3ccc(-c4ccc(-c5ccccc5)cc4)cc3)c3cccc(-c4ccccc4)c3)ccc1-2]

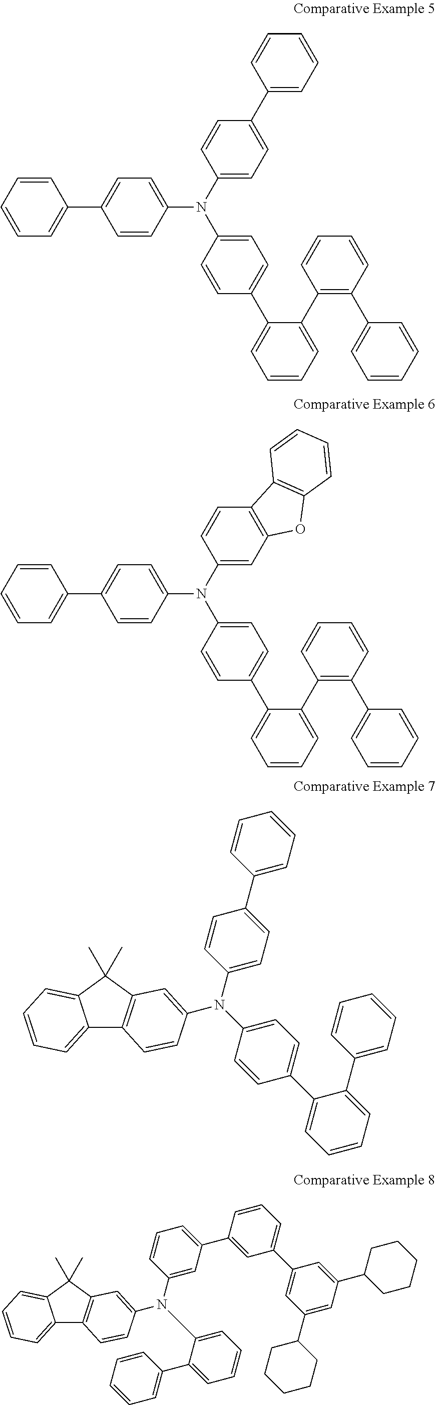

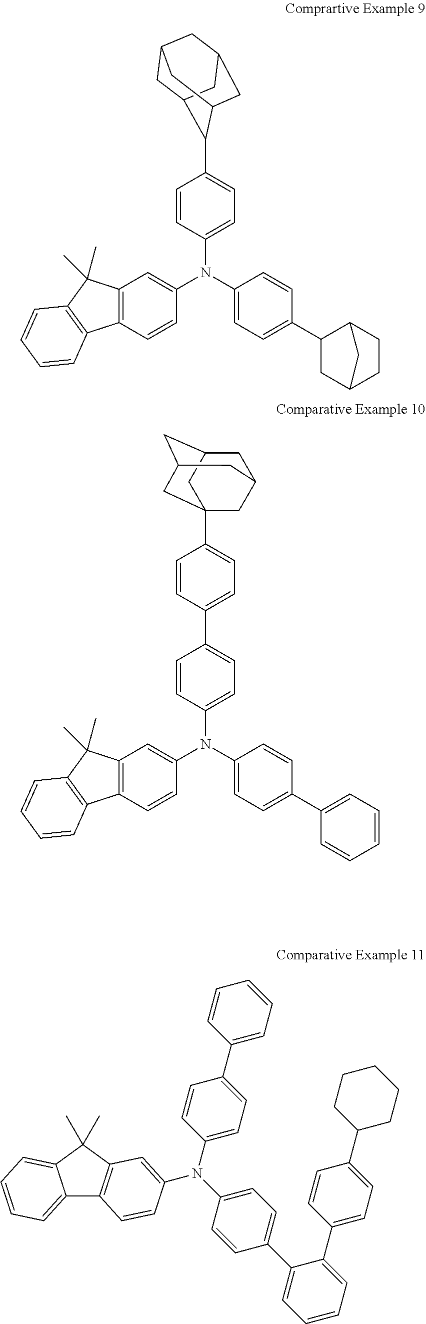 [SMILES: CC1(C)c2ccccc2-c2ccc(N(c3ccc(C4CC5CCC4C5)cc3)c3ccc(C4C5CC6CC(C5)CC4C6)cc3)cc21 ; CC1(C)c2ccccc2-c2ccc(N(c3ccc(-c4ccccc4)cc3)c3ccc(-c4ccc(C56CC7CC(CC(C7)C5)C6)cc4)cc3)cc21 ; CC1(C)c2ccccc2-c2ccc(N(c3ccc(-c4ccccc4)cc3)c3ccc(-c4ccccc4-c4ccc(C5CCCCC5)cc4)cc3)cc21]

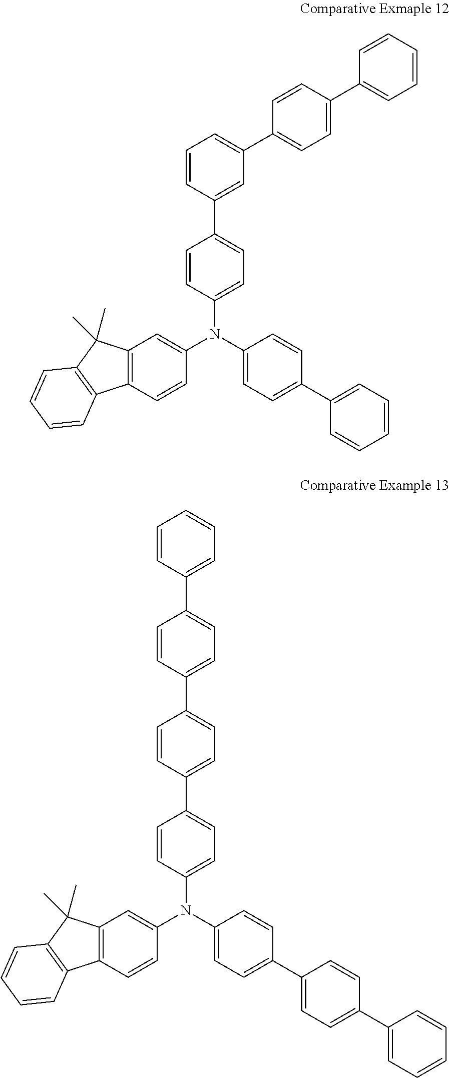 [SMILES: CC1(C)c2ccccc2-c2ccc(N(c3ccc(-c4ccccc4)cc3)c3ccc(-c4cccc(-c5ccc(-c6ccccc6)cc5)c4)cc3)cc21 ; CC1(C)c2ccccc2-c2ccc(N(c3ccc(-c4ccc(-c5ccccc5)cc4)cc3)c3ccc(-c4ccc(-c5ccc(-c6ccccc6)cc5)cc4)cc3)cc21]

### Examples 16 to 18 and Comparative Example 14

An organic light-emitting device of Example 16 was manufactured in substantially the same manner as in Example 1, except that, in forming a hole transport layer, Compound 1 was deposited to a thickness of 100 Å to form a first hole transport layer, HT01 was deposited to a thickness of 100 Å on the first hole transport layer to form a second hole transport layer, Compound 1 was deposited on the second hole transport layer to a thickness of 100 Å to form a third hole transport layer, thereby forming a hole transport layer.

In some embodiments, an organic light-emitting device of Example 17 was manufactured in substantially the same manner as in Example 16, except that Compound 268, HT01, and Compound 268 were respectively utilized for the first hole transport layer, the second hole transport layer, and the third hole transport layer, and an organic light-emitting device of Example 18 was manufactured in substantially the same manner as in Example 16, except that Compound 270, HT01, and Compound 270 were respectively utilized for the first hole transport layer, the second hole transport layer, and the third hole transport layer.

An organic light-emitting device of Comparative Example 14 was manufactured in substantially the same manner as in Example 16, except that Comparative Example Compound 9, HT01, and Comparative Example Compound 9 were respectively utilized for the first hole transport layer, the second hole transport layer, and the third hole transport layer.

### Evaluation Example 2

The driving voltage (V), current density (mA/cm²), luminance (cd/m²), luminescence efficiency (cd/A), and lifespan (hours) at 1,000 cd/m² of each of the organic light-emitting devices of Examples 1 to 18 and Comparative Examples 1 to 14 were measured utilizing a Keithley SMU 236 and a PR650 luminance meter. The results thereof are shown in Table 3.

TABLE 3

| | Hole | Driving | Current | | | | Half-life |
|---|---|---|---|---|---|---|---|
| | transport | voltage | density | Luminance | Efficiency | Emission | (hr @100 |
| | material | (V) | (mA/cm²) | (cd/m²) | (cd/A) | color | mA/cm²) |
| Example 1 | Compound 1 | 4.4 | 50 | 3650 | 7.3 | Blue | 380 |
| Example 2 | Compound 2 | 4.5 | 50 | 3700 | 7.4 | Blue | 360 |
| Example 3 | Compound 3 | 4.3 | 50 | 3720 | 7.44 | Blue | 395 |
| Example 4 | Compound 5 | 4.5 | 50 | 3730 | 7.46 | Blue | 390 |
| Example 5 | Compound 23 | 4.2 | 50 | 3850 | 7.7 | Blue | 415 |
| Example 6 | Compound 83 | 4.5 | 50 | 3900 | 7.8 | Blue | 410 |
| Example 7 | Compound 103 | 4.2 | 50 | 3690 | 7.38 | Blue | 385 |
| Example 8 | Compound 123 | 4.1 | 50 | 3730 | 7.46 | Blue | 405 |
| Example 9 | Compound 241 | 4.2 | 50 | 3700 | 7.4 | Blue | 395 |
| Example 10 | Compound 265 | 4.2 | 50 | 3920 | 7.84 | Blue | 398 |
| Example 11 | Compound 268 | 4.0 | 50 | 3950 | 7.9 | Blue | 410 |
| Example 12 | Compound 270 | 4.2 | 50 | 3900 | 7.8 | Blue | 410 |
| Example 13 | Compound 291 | 4.2 | 50 | 3850 | 7.7 | Blue | 402 |
| Example 14 | Compound 296 | 4.0 | 50 | 3900 | 7.8 | Blue | 405 |
| Example 15 | Compound 356 | 3.9 | 50 | 3850 | 7.7 | Blue | 400 |
| Example 16 | Compound 1, | 4.2 | 50 | 3990 | 7.98 | Blue | 420 |
| | HT01 | | | | | | |
| Example 17 | Compound 268, | 4.0 | 50 | 4050 | 8.1 | Blue | 430 |
| | HT01 | | | | | | |
| Example 18 | Compound 270, | 4.1 | 50 | 4150 | 8.3 | Blue | 440 |
| | HT01 | | | | | | |
| Comparative | Comparative | 7.01 | 50 | 2645 | 5.29 | Blue | 258 |
| Example 1 | Example | | | | | | |
| | Compound 1 | | | | | | |
| | (NPB) | | | | | | |
| Comparative | Comparative | 4.7 | 50 | 3200 | 6.4 | Blue | 300 |
| Example 2 | Example | | | | | | |
| | Compound 2 | | | | | | |
| | (HT01) | | | | | | |
| Comparative | Comparative | 8.5 | 50 | 3150 | 6.3 | Blue | 180 |
| Example 3 | Example | | | | | | |
| | Compound 3 | | | | | | |
| Comparative | Comparative | 5.4 | 50 | 3010 | 6.0 | Blue | 215 |
| Example 4 | Example | | | | | | |
| | Compound 4 | | | | | | |
| Comparative | Comparative | 6.2 | 50 | 3200 | 6.1 | Blue | 200 |
| Example 5 | Example | | | | | | |
| | Compound 5 | | | | | | |
| Comparative | Comparative | 5.7 | 50 | 3250 | 6.5 | Blue | 205 |
| Example 6 | Example | | | | | | |
| | Compound 6 | | | | | | |
| Comparative | Comparative | 4.8 | 50 | 2750 | 5.5 | Blue | 230 |
| Example 7 | Example | | | | | | |
| | Compound 7 | | | | | | |
| Comparative | Comparative | 6.2 | 50 | 3050 | 6.1 | Blue | 185 |
| Example 8 | Example | | | | | | |
| | Compound 8 | | | | | | |
| Comparative | Comparative | 6.1 | 50 | 3070 | 6.1 | Blue | 220 |
| Example 9 | Example | | | | | | |
| | Compound 9 | | | | | | |
| Comparative | Comparative | 5.6 | 50 | 3300 | 6.6 | Blue | 225 |
| Example 10 | Example | | | | | | |
| | Compound 10 | | | | | | |
| Comparative | Comparative | 4.8 | 50 | 3450 | 6.9 | Blue | 310 |
| Example 11 | Example | | | | | | |
| | Compound 11 | | | | | | |
| Comparative | Comparative | 5.8 | 50 | 3100 | 6.2 | Blue | 185 |
| Example 12 | Example | | | | | | |
| | Compound 12 | | | | | | |
| Comparative | Comparative | 5.4 | 50 | 3010 | 6.0 | Blue | 205 |
| Example 13 | Example | | | | | | |
| | Compound 13 | | | | | | |
| Comparative | Comparative | 4.9 | 50 | 3470 | 6.9 | Blue | 325 |
| Example 14 | Example | | | | | | |
| | Compound 9, | | | | | | |
| | HT01 | | | | | | |

As should be apparent from the foregoing description, when the amine-containing compound represented by Formula 1 is utilized, a light-emitting device and an electron apparatus including the light-emitting device may have a high efficiency and long lifespan.

In the present disclosure, it will be understood that the terms “comprise(s),” “include(s),” or “have/has” specify the presence of stated features, integers, steps, operations, elements, and/or components, but do not preclude the presence or addition of one or more other features, integers, steps, operations, elements, components, and/or groups thereof.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

In the present disclosure, although the terms “first,” “second,” “third,” “fourth,” etc., may be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

In the present disclosure, when particles are spherical, “diameter” indicates a particle diameter or an average particle diameter, and when the particles are non-spherical, the “diameter” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting device, the display device, the display apparatus, the electronic apparatus, the electronic equipment, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode facing the first electrode;
an interlayer between the first electrode and the second electrode, the interlayer comprising an emission layer; and
an amine-containing compound represented by Formula 1: 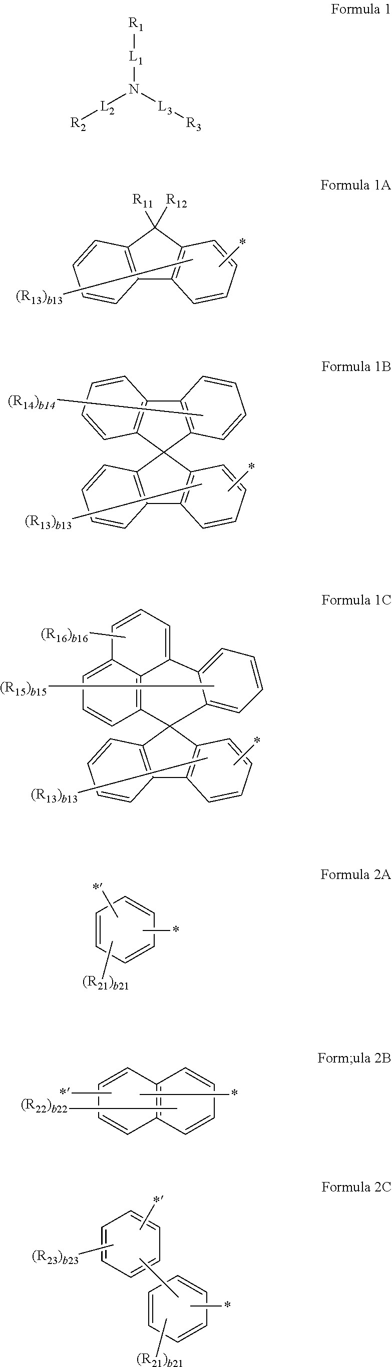 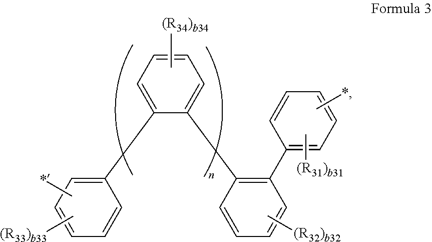
wherein, in Formula 1,
L₁ is a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
R₁ is a group represented by one selected from among Formulae 1A to 1C, wherein, in Formulae 1A and 1C, * indicates a binding site to L₁ in Formula 1,
L₂ is a group represented by one selected from among Formulae 2A to 2C, wherein, in Formulae 2A to 2C, * indicates a binding site to N in Formula 1, and indicates a binding site to R₂ in Formula 1,
R₂ is a C₃-C₁₀ cycloalkyl group unsubstituted or substituted with at least one R₁₀ₐ,
L₃ is a group represented by Formula 3, wherein, in Formula 3, * indicates a binding site to N in Formula 1, and *′ indicates a binding site to R₃ in Formula 1, in Formula 3, n is 0 or 1,
R₃, R₁₁ to R₁₆, R₂₁ to R₂₃, and R₃₁ to R₃₄ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ aryl alkyl group unsubstituted or substituted with at least one R₁₀ₐ, a C₂-C₆₀ heteroaryl alkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b13 and b15 are each an integer from 0 to 7,
b14 is an integer from 0 to 8,
b16 is an integer from 0 to 3,
b21, b23, and b31 to b34 are each independently an integer from 0 to 3,
b22 is an integer from 0 to 6, and
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The light-emitting device of claim 1, wherein:
the first electrode is an anode,
the second electrode is a cathode,
the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or any combination thereof, and
the electron transport region comprises a hole blocking layer, an electron transport layer, an electron injection layer, an electron control layer, or any combination thereof.

3. The light-emitting device of claim 1, wherein the amine-containing compound represented by Formula 1 is in the interlayer.

4. The light-emitting device of claim 2, wherein the amine-containing compound represented by Formula 1 is in the hole transport region.

5. The light-emitting device of claim 2, wherein the amine-containing compound is in the hole transport layer, and the hole transport layer is in direct contact with the emission layer.

6. The light-emitting device of claim 2, wherein the light-emitting device further comprises a capping layer outside the second electrode, and the amine-containing compound is in the capping layer.

7. An electronic apparatus comprising the light-emitting device of claim 1.

8. The electronic apparatus of claim 7, further comprising a color filter, a color-conversion layer, a touchscreen layer, a polarizing layer, or any combination thereof.

9. An electronic equipment comprising the light-emitting device of claim 1.

10. The electronic equipment of claim 9, wherein the electronic equipment is at least one selected from a flat panel display, a curved display, a computer monitor, a medical monitor, a television, an advertisement board, an indoor or outdoor lighting, and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a 3D display, a virtual or augmented reality display, a vehicle, a video wall comprising multiple displays tiled together, a theater or stadium screen, a phototherapy device, and a signage.

11. An amine-containing compound represented by Formula 1: 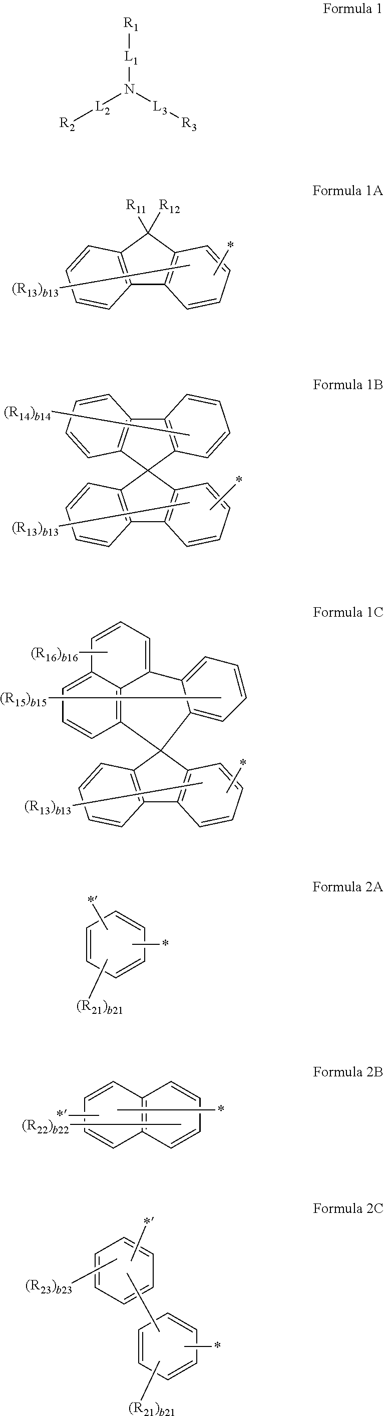 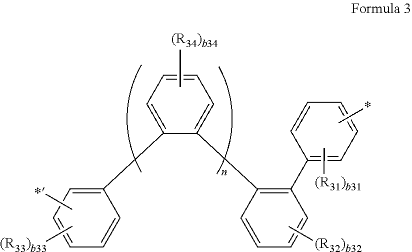
wherein, in Formula 1,
L₁ is a single bond, a C₃-C₆₀ carbocyclic group unsubstituted or substituted with at least one R₁₀ₐ, or a C₁-C₆₀ heterocyclic group unsubstituted or substituted with at least one R₁₀ₐ,
R₁ is a group represented by one selected from among Formulae 1A to 1C, wherein, in Formulae 1A and 1 C, * indicates a binding site to L₁ in Formula 1,
L₂ is a group represented by one selected from among Formulae 2A to 2C, wherein, in Formulae 2A to 2C, * indicates a binding site to N in Formula 1, and indicates a binding site to R₂ in Formula 1,
R₂ is a C₃-C₁₀ cycloalkyl group unsubstituted or substituted with at least one R₁₀ₐ,
L₃ is a group represented by Formula 3, wherein, in Formula 3, * indicates a binding site to N in Formula 1, and *′ indicates a binding site to R₃ in Formula 1,
in Formula 3, n is 0 or 1,
R₃, R₁₁ to R₁₆, R₂₁ to R₂₃, and R₃₁ to R₃₄ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ aryl alkyl group unsubstituted or substituted with at least one R₁₀ₐ, a C₂-C₆₀ heteroaryl alkyl group unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b13 and b15 are each an integer from 0 to 7,
b14 is an integer from 0 to 8,
b16 is an integer from 0 to 3,
b21, b23, and b31 to b34 are each independently an integer from 0 to 3,
b22 is an integer from 0 to 6, and
R₁₀ₐ is:
deuterium, —F, —CI, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —CI, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —CI, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
wherein Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, or C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

12. The amine-containing compound of claim 11, wherein L₁ is a single bond.

13. The amine-containing compound of claim 11, wherein R₁ is a group represented by one selected from Formulae 1-1 to 1-12: 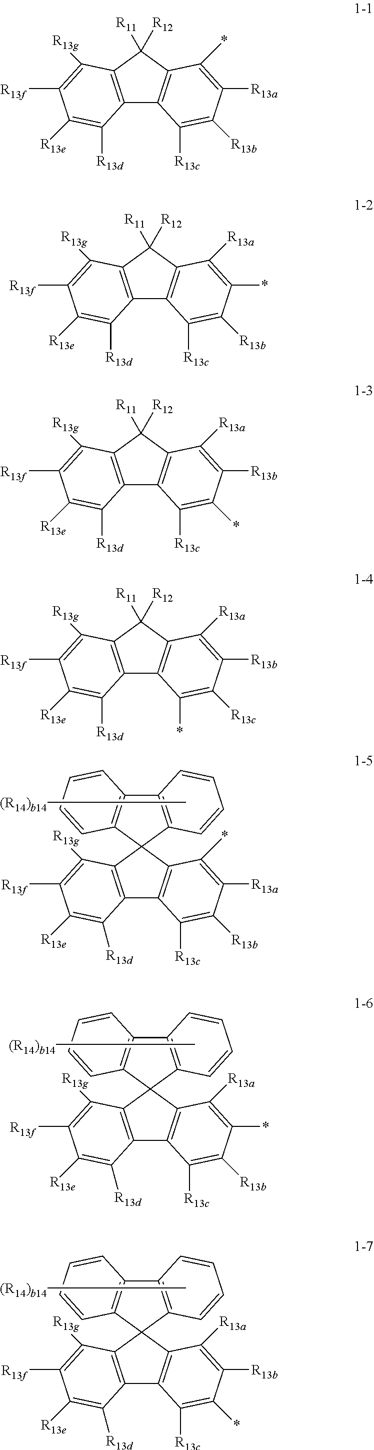 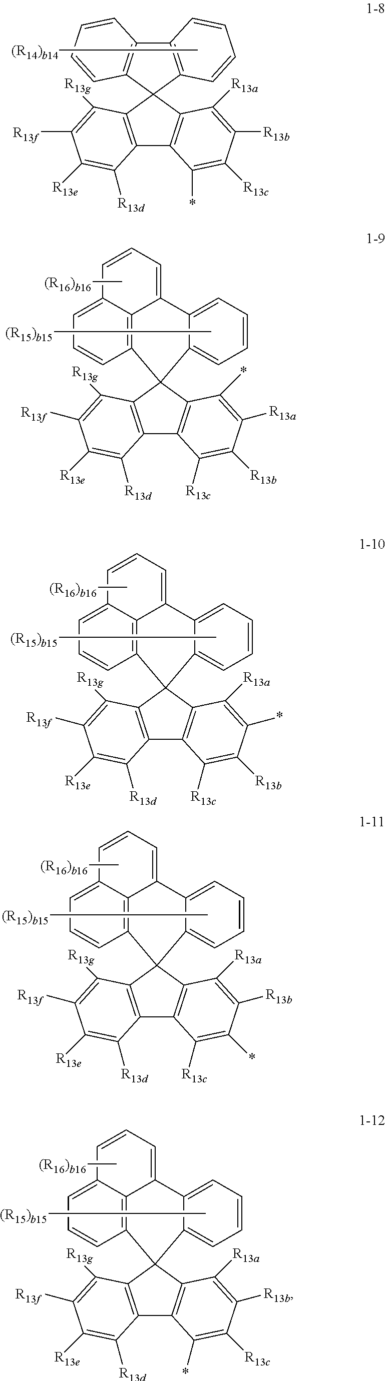
wherein, in Formulae 1-1 to 1-12,
R₁₁, R₁₂, R₁₄, R₁₅, R₁₆, b14, b15, and b16 are the same as defined with respect to R₁₁, R₁₂, R₁₄, R₁₅, R₁₆, b14, b15, and b16 in Formula 1, respectively,
R₁₃ₐ, R_{13b}, R_{13c}, R_{13d}, R₁₃ₑ, R_{13f}, and R_{13g} are each independently the same as defined with respect to R₁₃ in Formula 1, and
* indicates a binding site to L₁ in Formula 1.

14. The amine-containing compound of claim 11, wherein L₂ is a group represented by one selected from among Formulae 2-1 to 2-17: 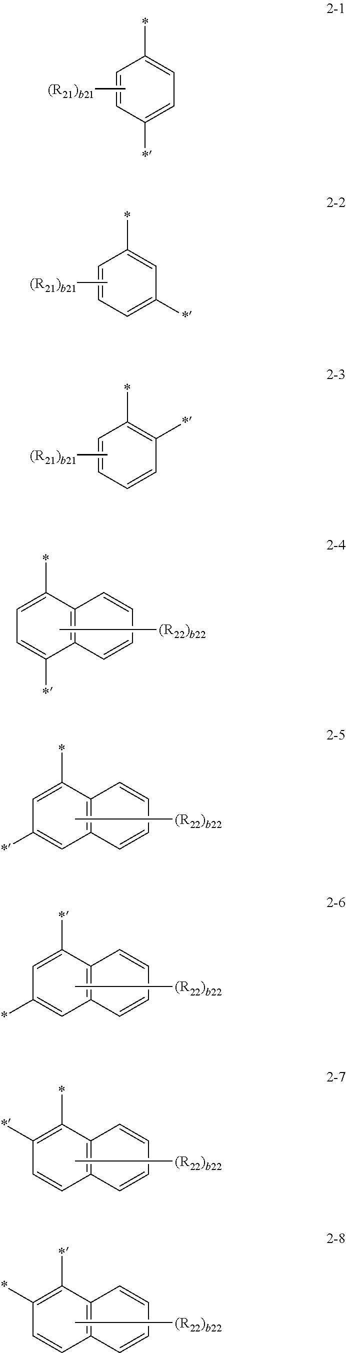 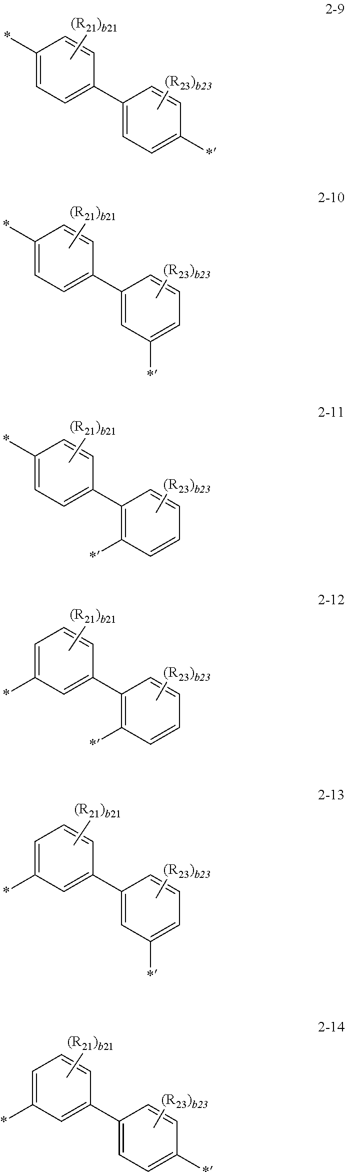 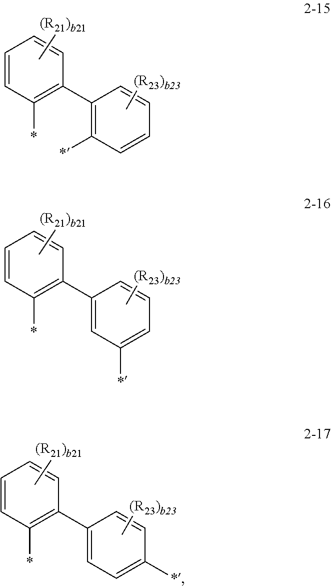
wherein, in Formulae 2-1 to 2-17,
R₂₁ to R₂₃ and b21 to b23 are the same as defined with respect to R₂₁ to R₂₃ and b21 to b23 in Formula 1, respectively,
* indicates a binding site to N in Formula 1, and
* indicates a binding site to R₂ in Formula 1.

15. The amine-containing compound of claim 11, wherein R₂ is one selected from among Formulae 2-A to 2-G: 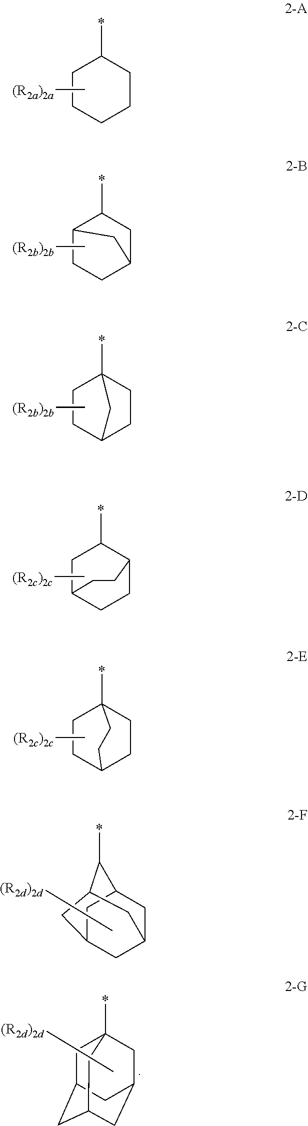
and
wherein, in Formulae 2-A to 2-G,
2a and 2b are each an integer from 0 to 11,
2c is an integer from 0 to 13,
2d is an integer from 0 to 15,
R₂ₐ to R_{2d} are each independently the same as defined with respect to R₃ in Formula 1, and
* indicates a binding site to L₂ in Formula 1.

16. The amine-containing compound of claim 11, wherein R₂ is a cyclopropyl group, a cyclobutyl group, a cyclopentyl group, a cyclohexyl group, a cycloheptyl group, a cyclooctyl group, an adamantanyl group, a norbornanyl group, a bicyclo[1.1.1]pentyl group, a bicyclo[2.1.1]hexyl group, or a bicyclo[2.2.2]octyl group, each unsubstituted or substituted with at least one R₁₀ₐ.

17. The amine-containing compound of claim 11, wherein L₃ is one selected from among Formulae 3-1 to 3-18: 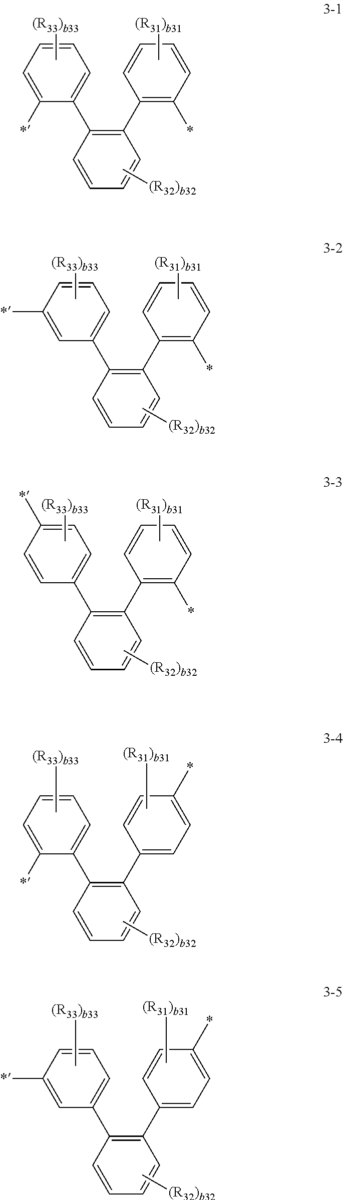 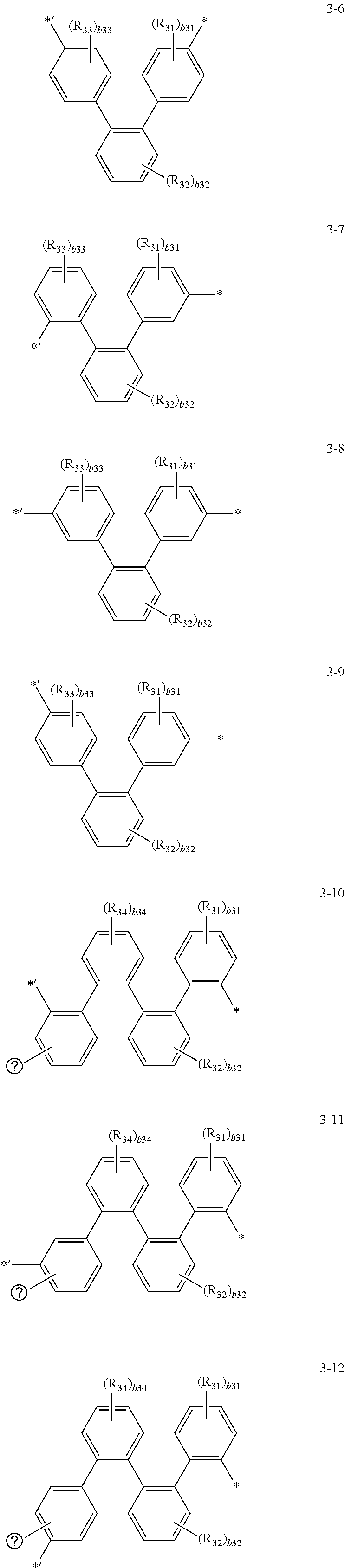 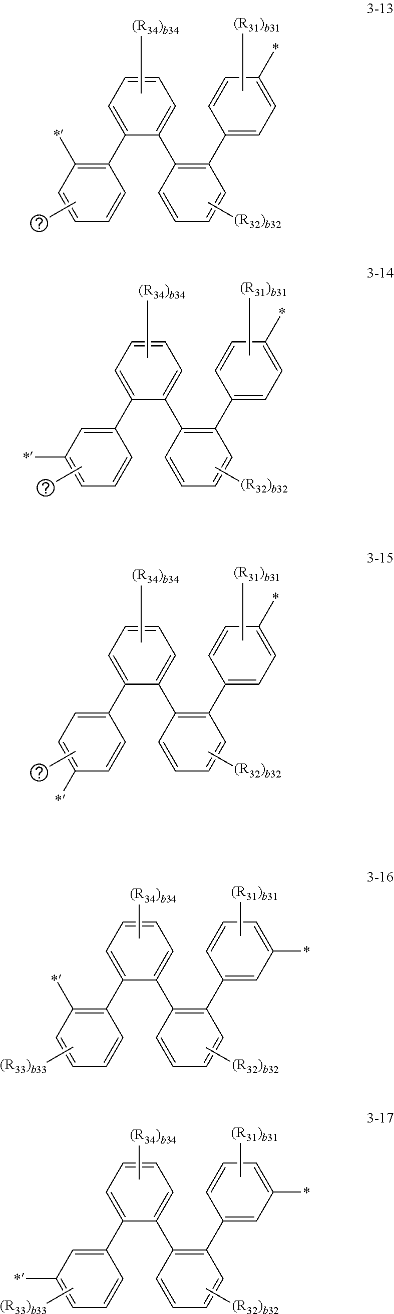 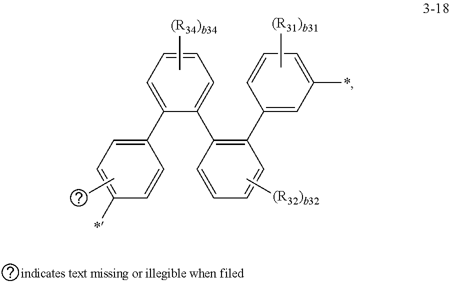
wherein, in Formulae 3-1 to 3-18,
R₃₁ to R₃₄ and b31 to b34 are each independently the same as defined in Formula 1, and
* indicates a binding site to N in Formula 1, and *′ indicates a binding site to R₃ in Formula 1.

18. The amine-containing compound of claim 11, wherein R₃ is a C₃-C₁₀ cycloalkyl group unsubstituted or substituted with at least one R₁₀ₐ.

19. The amine-containing compound of claim 11, wherein R₁₁ to R₁₆ are each independently:
hydrogen, deuterium, —F, or a cyano group;
a C₁-C₂₀ alkyl group unsubstituted or substituted with hydrogen, deuterium, —F, a cyano group, or any combination thereof; or
a benzene group, a pyridine group, a dibenzofuran group, a dibenzothiophene group, a carbazole group, an azadibenzofuran group, an azadibenzothiophene group, or an azacarbazole group, each unsubstituted or substituted with hydrogen, deuterium, —F, a cyano group, a C₁-C₂₀ alkyl group, a deuterated C₁-C₂₀ alkyl group, a fluorinated C₁-C₂₀ alkyl group, a phenyl group, deuterated a phenyl group, a fluorinated phenyl group, a (C₁-C₂₀ alkyl)phenyl group, a biphenyl group, a dibenzofuranyl group, a dibenzothiophenyl group, a carbazolyl group, a fluorenyl group, a dibenzosilolyl group, or any combination thereof.

20. The amine-containing compound of claim 11, wherein the amine-containing compound represented by Formula 1 is one selected from among Compounds 1 to 375: 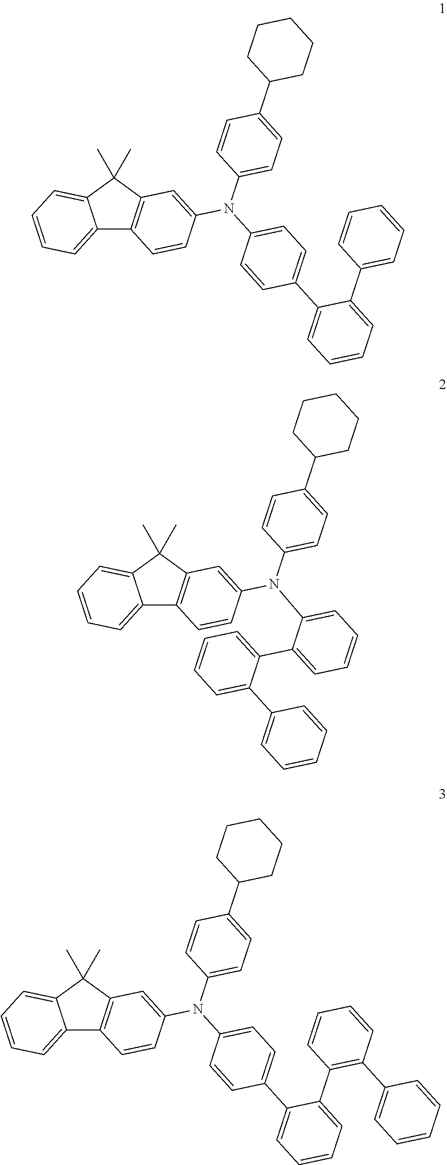 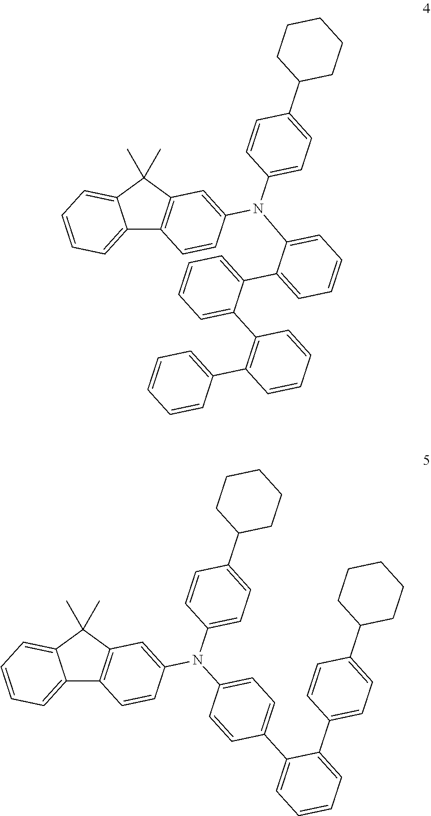 [SMILES: CC1(C)c2ccccc2-c2ccc(N(c3ccc(C4CCCCC4)cc3)c3ccccc3-c3ccccc3-c3ccccc3-c3ccccc3)cc21 ; CC1(C)c2ccccc2-c2ccc(N(c3ccc(-c4ccccc4-c4ccc(C5CCCCC5)cc4)cc3)c3ccc(C4CCCCC4)cc3)cc21] 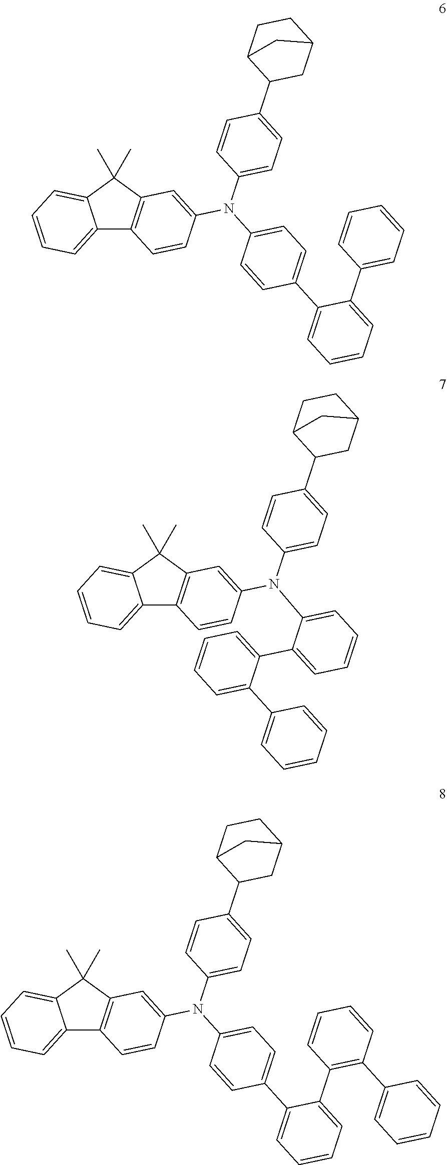 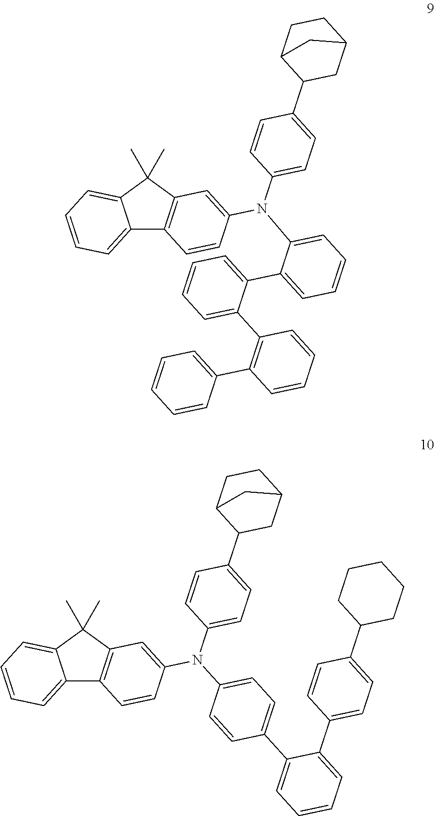 [SMILES: CC1(C)c2ccccc2-c2ccc(N(c3ccc(C4CC5CCC4C5)cc3)c3ccccc3-c3ccccc3-c3ccccc3-c3ccccc3)cc21 ; CC1(C)c2ccccc2-c2ccc(N(c3ccc(-c4ccccc4-c4ccc(C5CCCCC5)cc4)cc3)c3ccc(C4CC5CCC4C5)cc3)cc21] 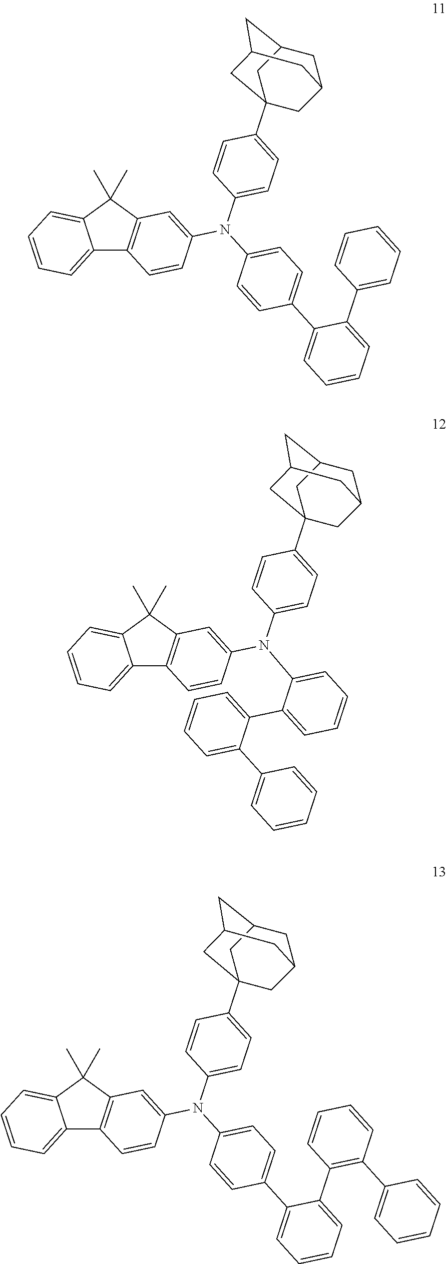 [SMILES: CC1(C)c2ccccc2-c2ccc(N(c3ccc(-c4ccccc4-c4ccccc4)cc3)c3ccc(C45CC6CC(CC(C6)C4)C5)cc3)cc21 ; CC1(C)c2ccccc2-c2ccc(N(c3ccc(C45CC6CC(CC(C6)C4)C5)cc3)c3ccccc3-c3ccccc3-c3ccccc3)cc21 ; CC1(C)c2ccccc2-c2ccc(N(c3ccc(-c4ccccc4-c4ccccc4-c4ccccc4)cc3)c3ccc(C45CC6CC(CC(C6)C4)C5)cc3)cc21] 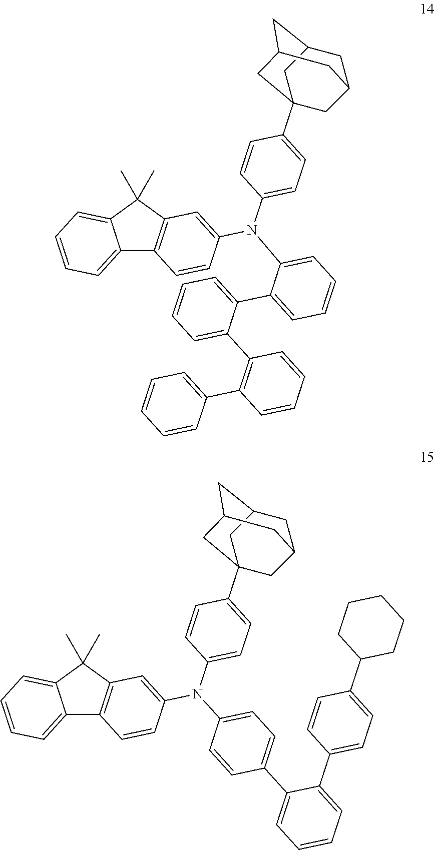 [SMILES: CC1(C)c2ccccc2-c2ccc(N(c3ccc(C45CC6CC(CC(C6)C4)C5)cc3)c3ccccc3-c3ccccc3-c3ccccc3-c3ccccc3)cc21 ; CC1(C)c2ccccc2-c2ccc(N(c3ccc(-c4ccccc4-c4ccc(C5CCCCC5)cc4)cc3)c3ccc(C45CC6CC(CC(C6)C4)C5)cc3)cc21] 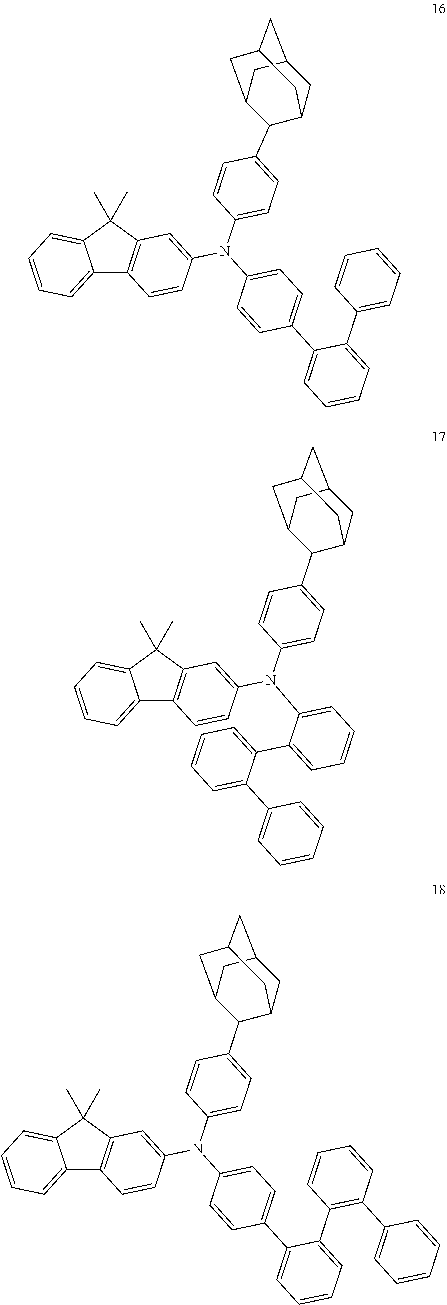 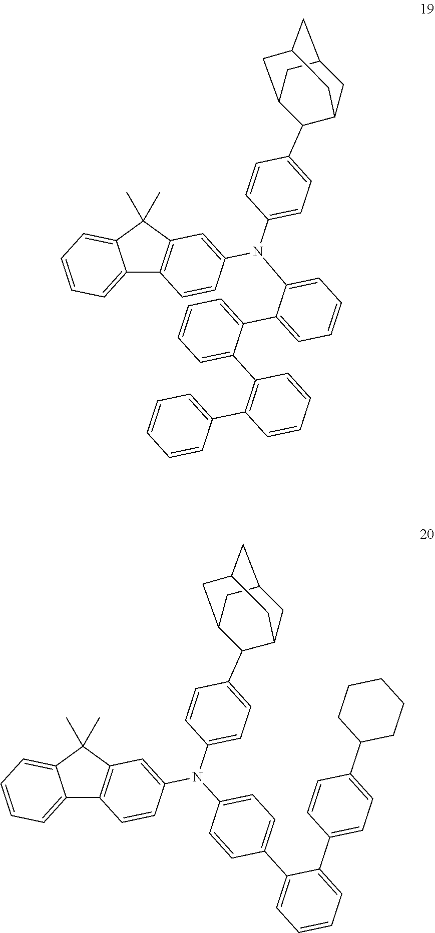 [SMILES: CC1(C)c2ccccc2-c2ccc(N(c3ccc(C4C5CC6CC(C5)CC4C6)cc3)c3ccccc3-c3ccccc3-c3ccccc3-c3ccccc3)cc21 ; CC1(C)c2ccccc2-c2ccc(N(c3ccc(-c4ccccc4-c4ccc(C5CCCCC5)cc4)cc3)c3ccc(C4C5CC6CC(C5)CC4C6)cc3)cc21] 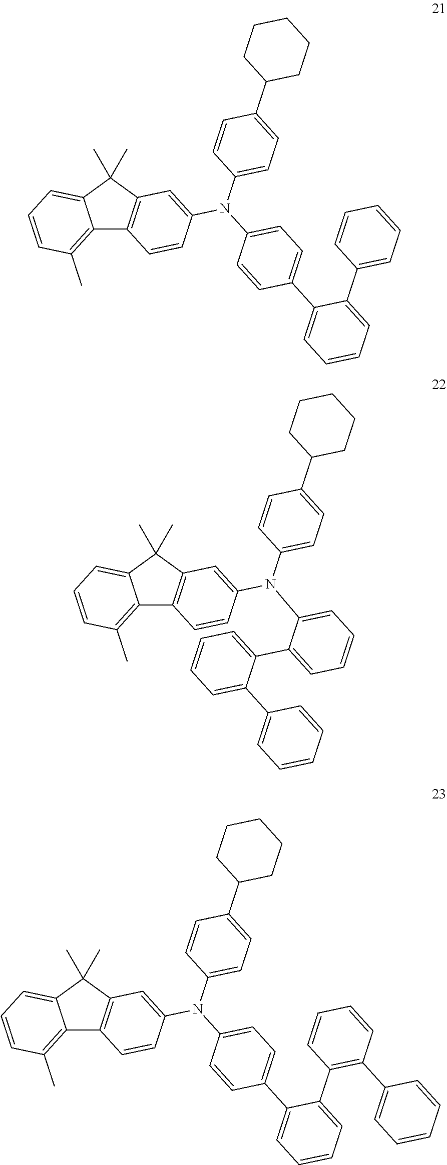 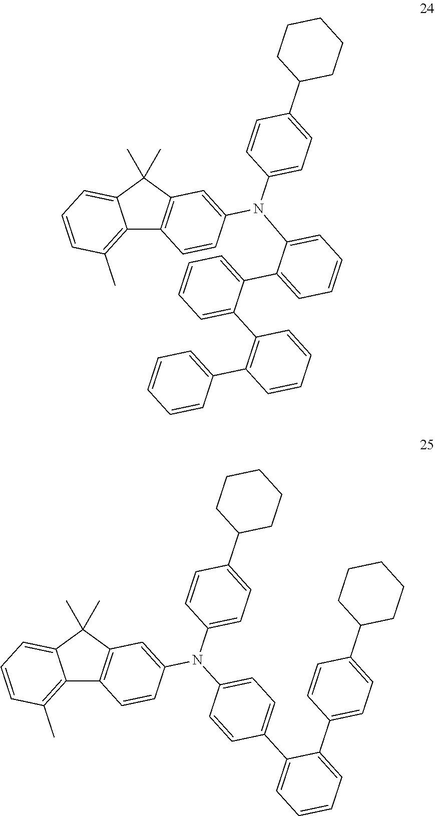 [SMILES: Cc1cccc2c1-c1ccc(N(c3ccc(C4CCCCC4)cc3)c3ccccc3-c3ccccc3-c3ccccc3-c3ccccc3)cc1C2(C)C ; Cc1cccc2c1-c1ccc(N(c3ccc(-c4ccccc4-c4ccc(C5CCCCC5)cc4)cc3)c3ccc(C4CCCCC4)cc3)cc1C2(C)C] 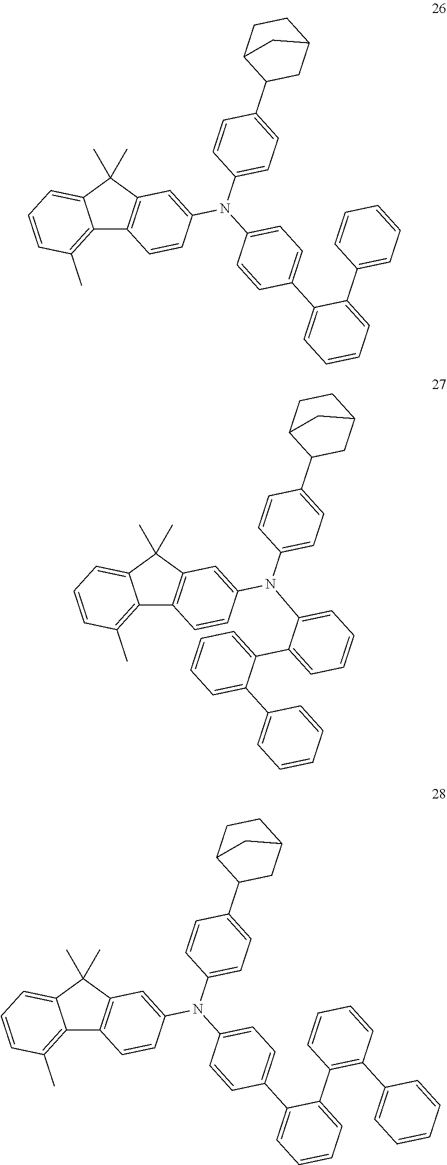 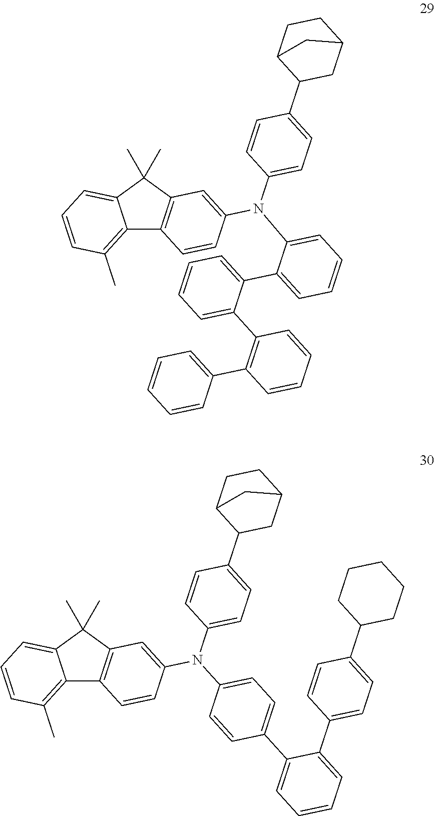 [SMILES: Cc1cccc2c1-c1ccc(N(c3ccc(C4CC5CCC4C5)cc3)c3ccccc3-c3ccccc3-c3ccccc3-c3ccccc3)cc1C2(C)C ; Cc1cccc2c1-c1ccc(N(c3ccc(-c4ccccc4-c4ccc(C5CCCCC5)cc4)cc3)c3ccc(C4CC5CCC4C5)cc3)cc1C2(C)C] 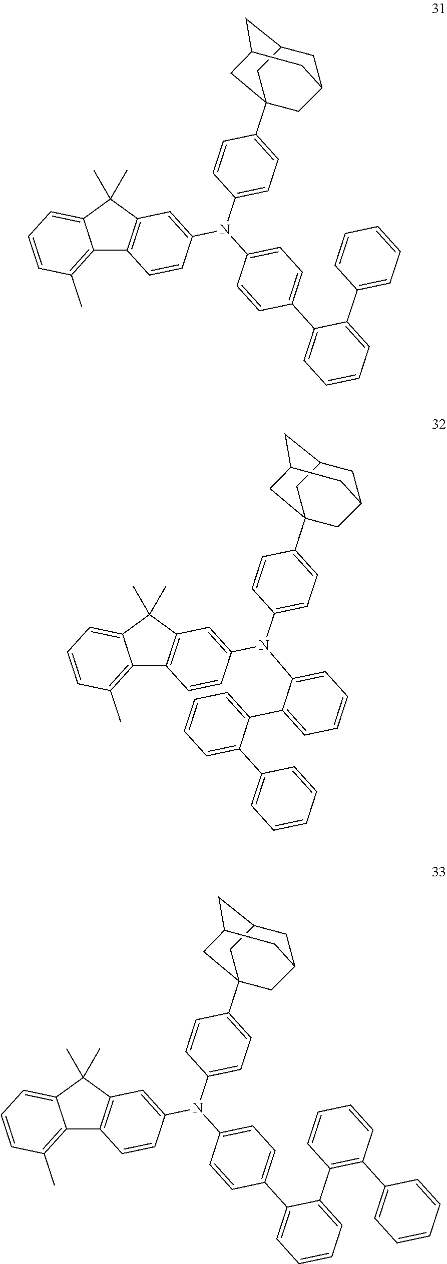 [SMILES: Cc1cccc2c1-c1ccc(N(c3ccc(-c4ccccc4-c4ccccc4)cc3)c3ccc(C45CC6CC(CC(C6)C4)C5)cc3)cc1C2(C)C ; Cc1cccc2c1-c1ccc(N(c3ccc(C45CC6CC(CC(C6)C4)C5)cc3)c3ccccc3-c3ccccc3-c3ccccc3)cc1C2(C)C ; Cc1cccc2c1-c1ccc(N(c3ccc(-c4ccccc4-c4ccccc4-c4ccccc4)cc3)c3ccc(C45CC6CC(CC(C6)C4)C5)cc3)cc1C2(C)C] 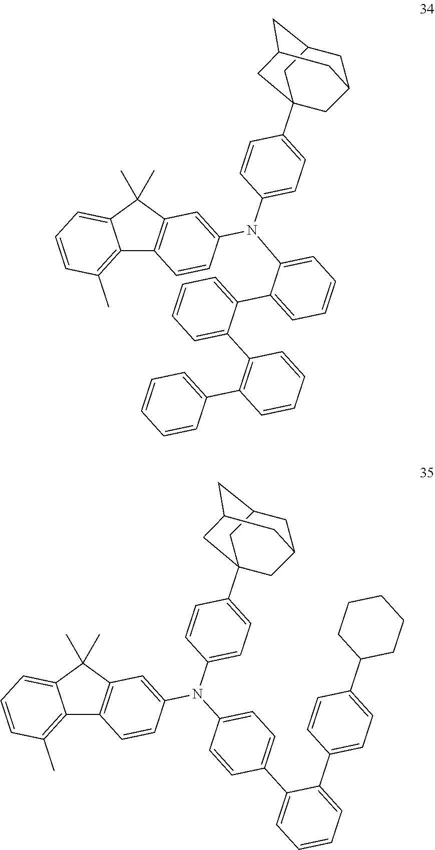 [SMILES: Cc1cccc2c1-c1ccc(N(c3ccc(C45CC6CC(CC(C6)C4)C5)cc3)c3ccccc3-c3ccccc3-c3ccccc3-c3ccccc3)cc1C2(C)C ; Cc1cccc2c1-c1ccc(N(c3ccc(-c4ccccc4-c4ccc(C5CCCCC5)cc4)cc3)c3ccc(C45CC6CC(CC(C6)C4)C5)cc3)cc1C2(C)C] 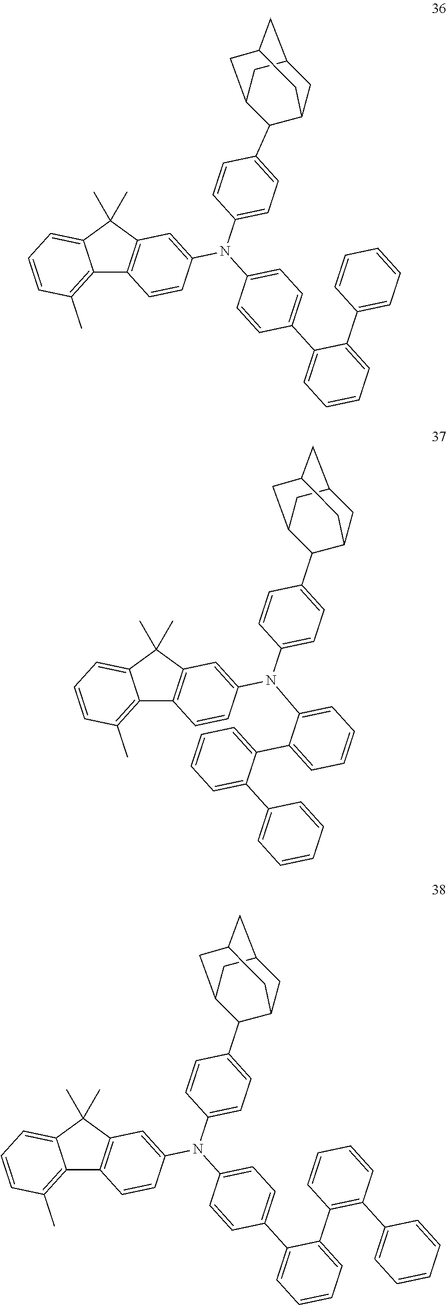 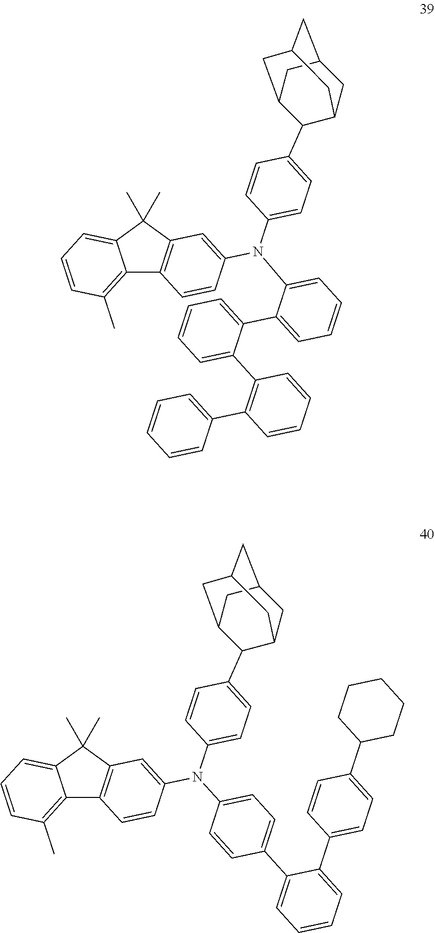 [SMILES: Cc1cccc2c1-c1ccc(N(c3ccc(C4C5CC6CC(C5)CC4C6)cc3)c3ccccc3-c3ccccc3-c3ccccc3-c3ccccc3)cc1C2(C)C ; Cc1cccc2c1-c1ccc(N(c3ccc(-c4ccccc4-c4ccc(C5CCCCC5)cc4)cc3)c3ccc(C4C5CC6CC(C5)CC4C6)cc3)cc1C2(C)C] 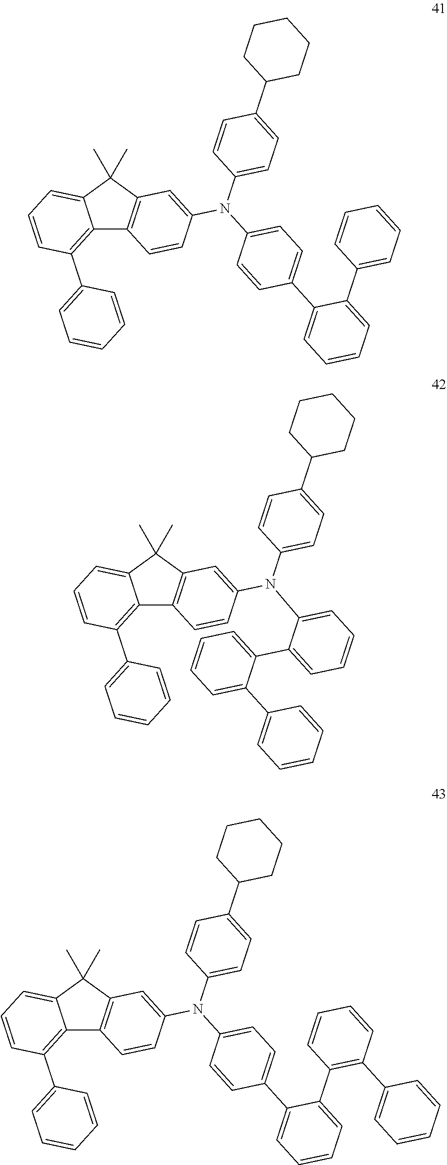 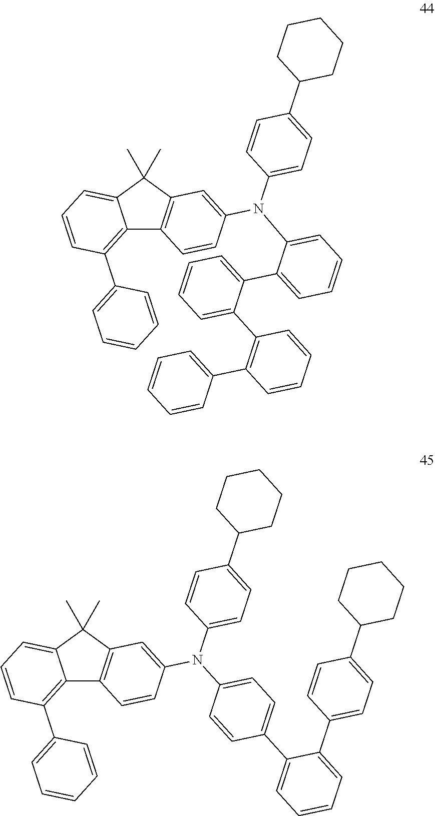 [SMILES: CC1(C)c2cc(N(c3ccc(C4CCCCC4)cc3)c3ccccc3-c3ccccc3-c3ccccc3-c3ccccc3)ccc2-c2c(-c3ccccc3)cccc21 ; CC1(C)c2cc(N(c3ccc(-c4ccccc4-c4ccc(C5CCCCC5)cc4)cc3)c3ccc(C4CCCCC4)cc3)ccc2-c2c(-c3ccccc3)cccc21] 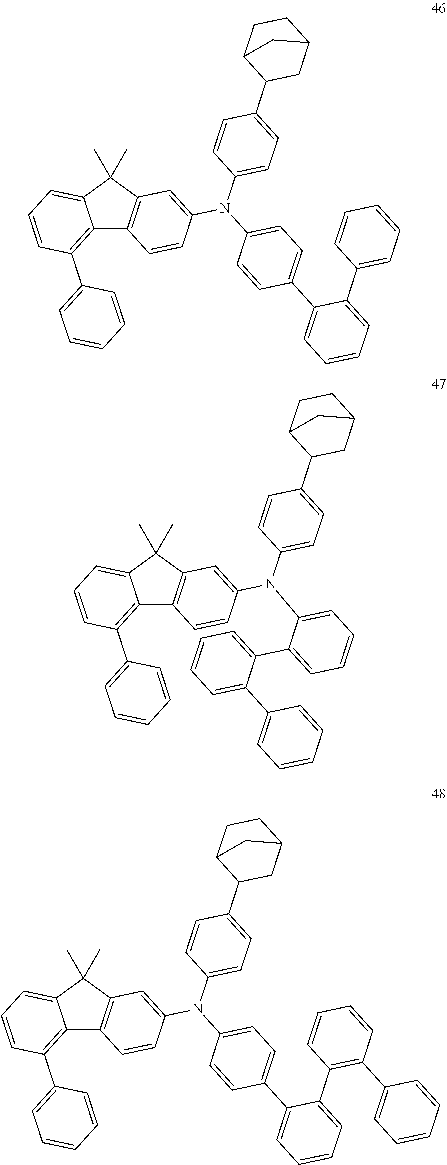 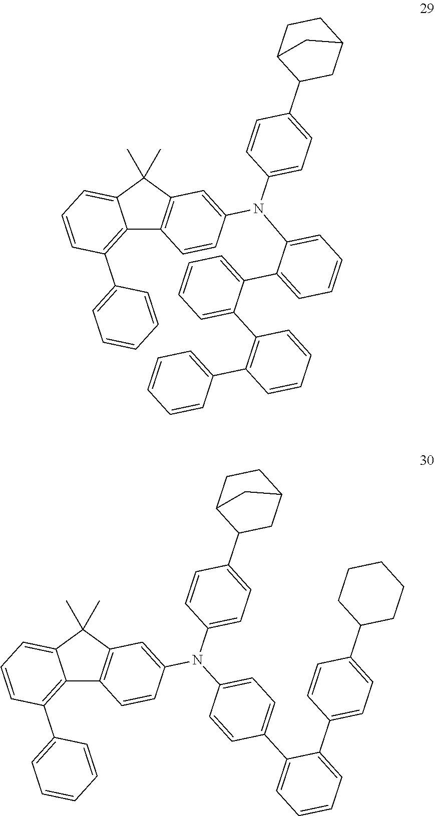 [SMILES: CC1(C)c2cc(N(c3ccc(C4CC5CCC4C5)cc3)c3ccccc3-c3ccccc3-c3ccccc3-c3ccccc3)ccc2-c2c(-c3ccccc3)cccc21 ; CC1(C)c2cc(N(c3ccc(-c4ccccc4-c4ccc(C5CCCCC5)cc4)cc3)c3ccc(C4CC5CCC4C5)cc3)ccc2-c2c(-c3ccccc3)cccc21] 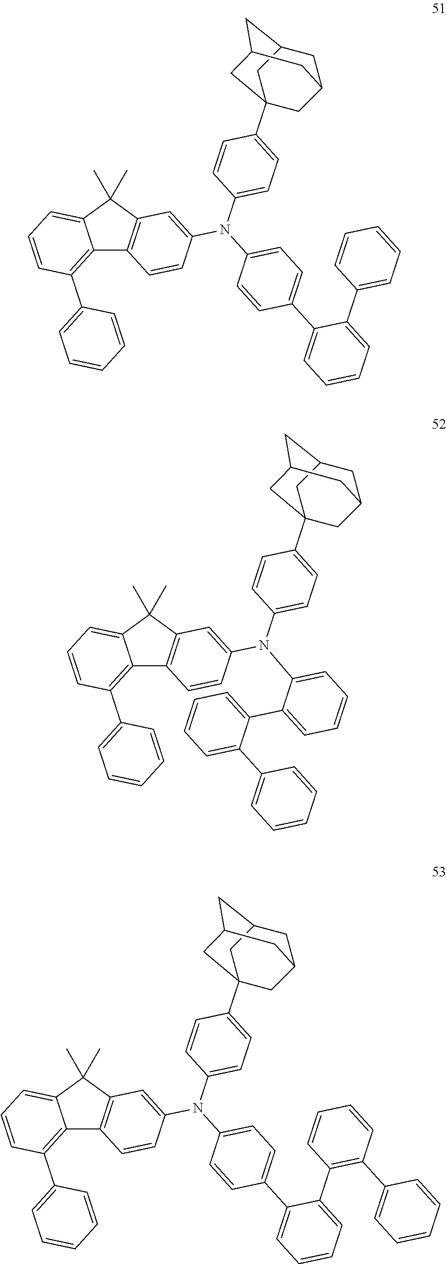 [SMILES: CC1(C)c2cc(N(c3ccc(-c4ccccc4-c4ccccc4)cc3)c3ccc(C45CC6CC(CC(C6)C4)C5)cc3)ccc2-c2c(-c3ccccc3)cccc21 ; CC1(C)c2cc(N(c3ccc(C45CC6CC(CC(C6)C4)C5)cc3)c3ccccc3-c3ccccc3-c3ccccc3)ccc2-c2c(-c3ccccc3)cccc21 ; CC1(C)c2cc(N(c3ccc(-c4ccccc4-c4ccccc4-c4ccccc4)cc3)c3ccc(C45CC6CC(CC(C6)C4)C5)cc3)ccc2-c2c(-c3ccccc3)cccc21] 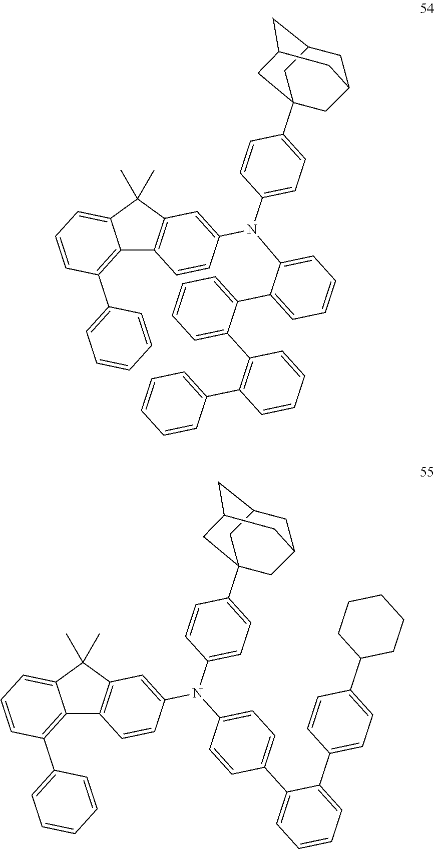 [SMILES: CC1(C)c2cc(N(c3ccc(C45CC6CC(CC(C6)C4)C5)cc3)c3ccccc3-c3ccccc3-c3ccccc3-c3ccccc3)ccc2-c2c(-c3ccccc3)cccc21 ; CC1(C)c2cc(N(c3ccc(-c4ccccc4-c4ccc(C5CCCCC5)cc4)cc3)c3ccc(C45CC6CC(CC(C6)C4)C5)cc3)ccc2-c2c(-c3ccccc3)cccc21] 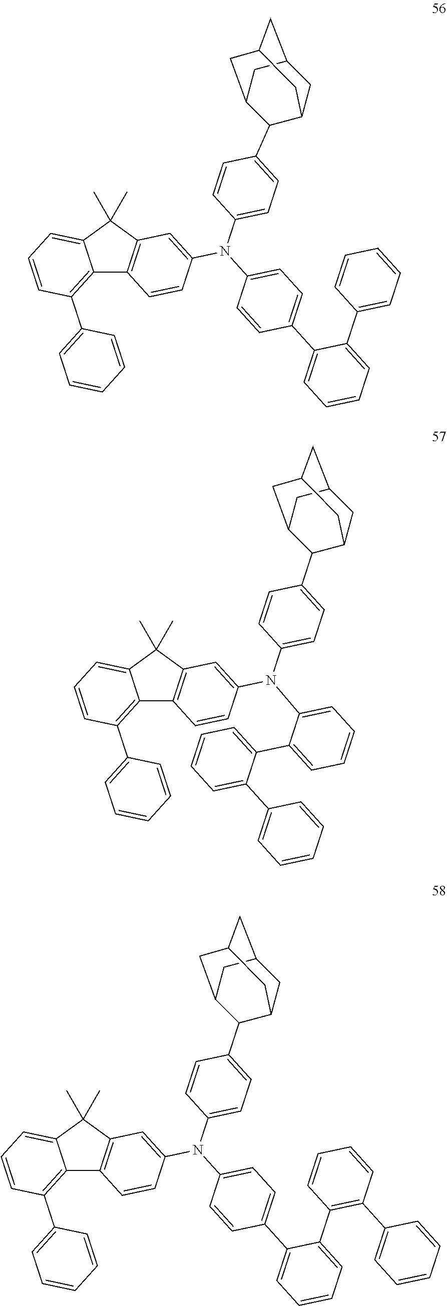 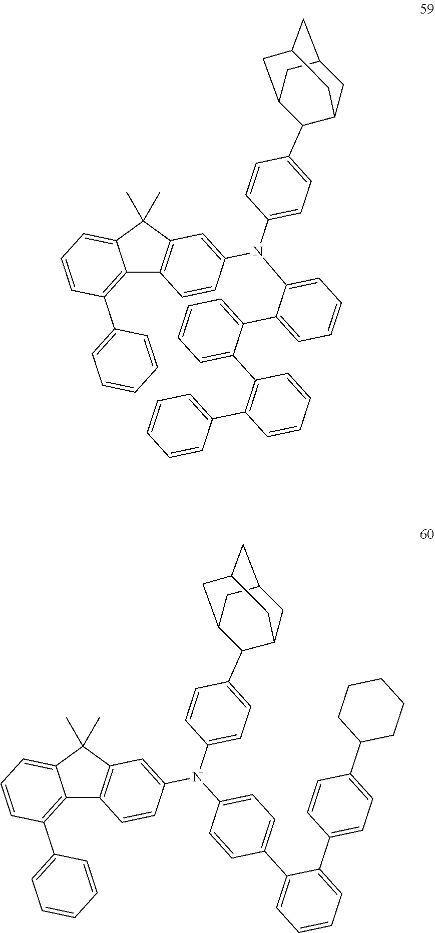 [SMILES: CC1(C)c2cc(N(c3ccc(C4C5CC6CC(C5)CC4C6)cc3)c3ccccc3-c3ccccc3-c3ccccc3-c3ccccc3)ccc2-c2c(-c3ccccc3)cccc21 ; CC1(C)c2cc(N(c3ccc(-c4ccccc4-c4ccc(C5CCCCC5)cc4)cc3)c3ccc(C4C5CC6CC(C5)CC4C6)cc3)ccc2-c2c(-c3ccccc3)cccc21] 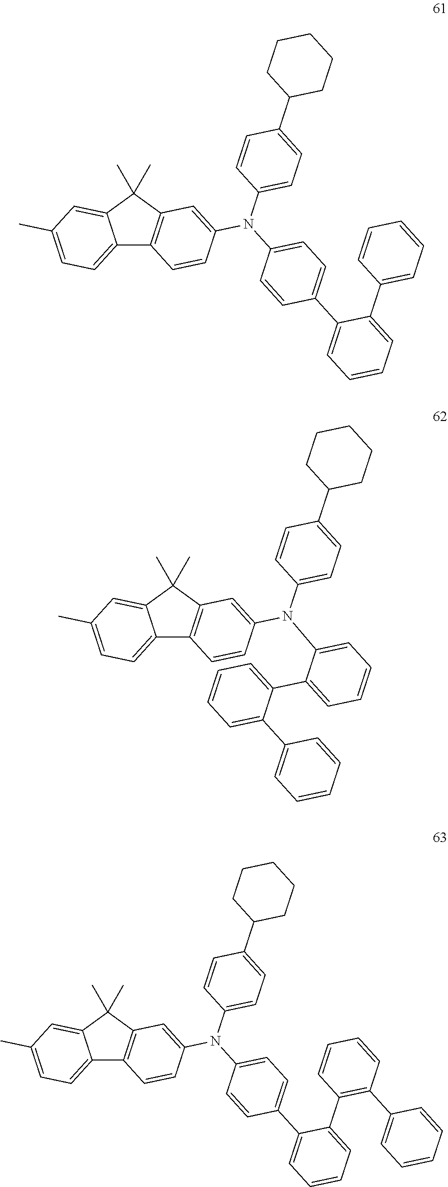 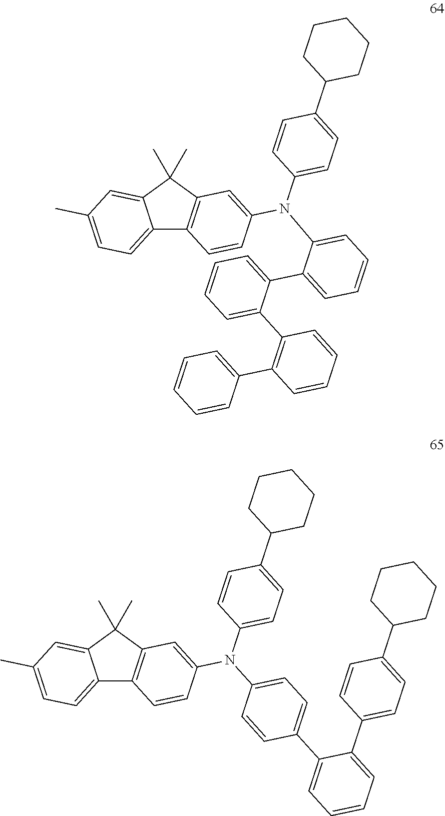 [SMILES: Cc1ccc2c(c1)C(C)(C)c1cc(N(c3ccc(C4CCCCC4)cc3)c3ccccc3-c3ccccc3-c3ccccc3-c3ccccc3)ccc1-2 ; Cc1ccc2c(c1)C(C)(C)c1cc(N(c3ccc(-c4ccccc4-c4ccc(C5CCCCC5)cc4)cc3)c3ccc(C4CCCCC4)cc3)ccc1-2] 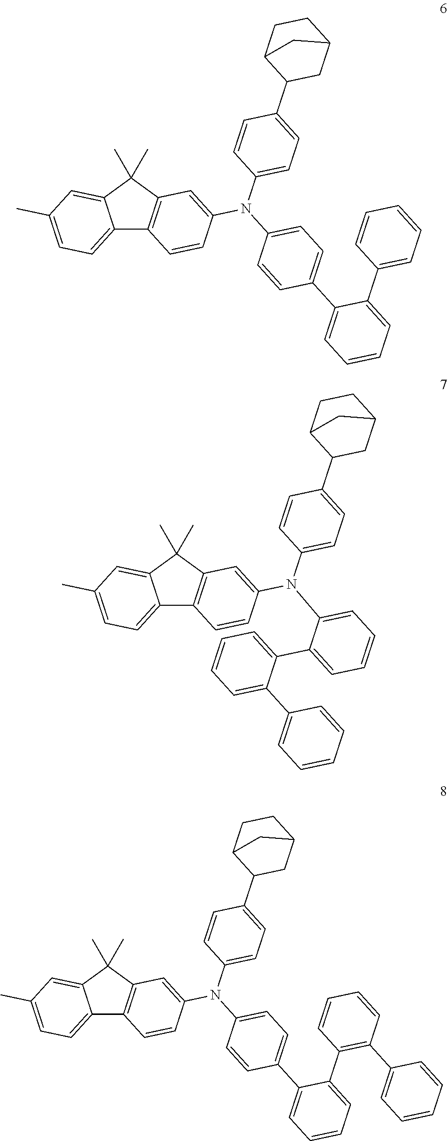 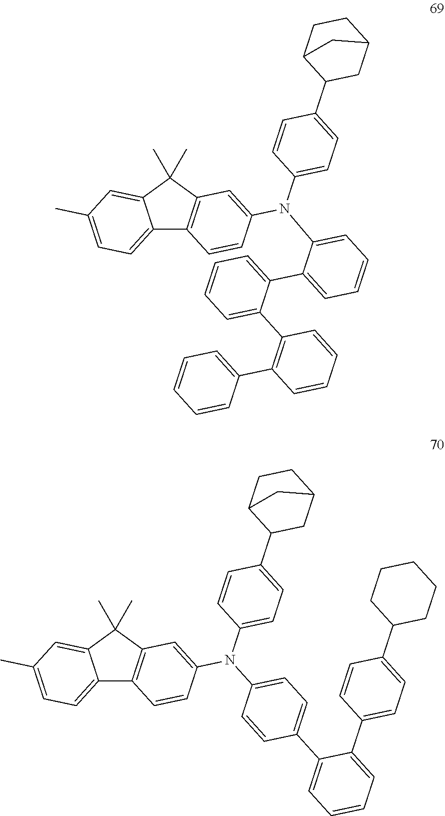 [SMILES: Cc1ccc2c(c1)C(C)(C)c1cc(N(c3ccc(C4CC5CCC4C5)cc3)c3ccccc3-c3ccccc3-c3ccccc3-c3ccccc3)ccc1-2 ; Cc1ccc2c(c1)C(C)(C)c1cc(N(c3ccc(-c4ccccc4-c4ccc(C5CCCCC5)cc4)cc3)c3ccc(C4CC5CCC4C5)cc3)ccc1-2] 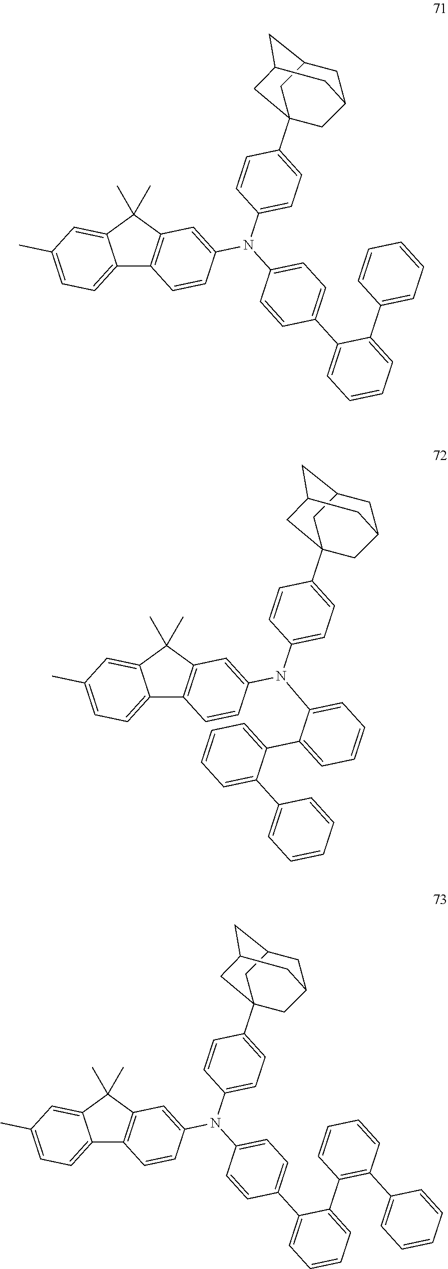 [SMILES: Cc1ccc2c(c1)C(C)(C)c1cc(N(c3ccc(-c4ccccc4-c4ccccc4)cc3)c3ccc(C45CC6CC(CC(C6)C4)C5)cc3)ccc1-2 ; Cc1ccc2c(c1)C(C)(C)c1cc(N(c3ccc(C45CC6CC(CC(C6)C4)C5)cc3)c3ccccc3-c3ccccc3-c3ccccc3)ccc1-2 ; Cc1ccc2c(c1)C(C)(C)c1cc(N(c3ccc(-c4ccccc4-c4ccccc4-c4ccccc4)cc3)c3ccc(C45CC6CC(CC(C6)C4)C5)cc3)ccc1-2] 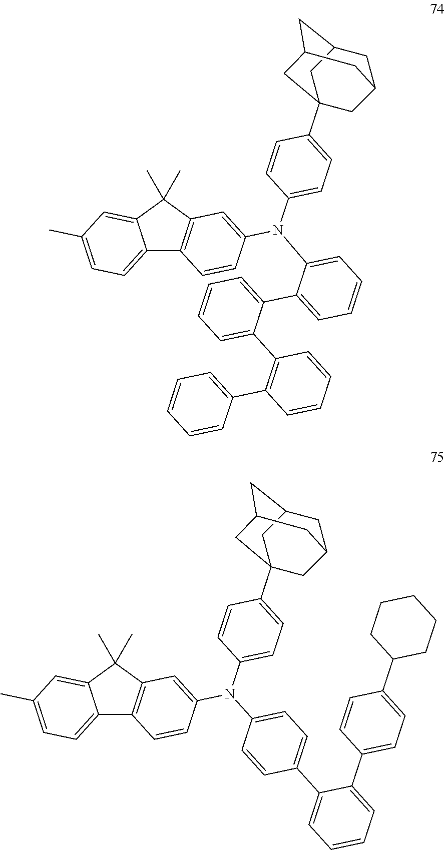 [SMILES: Cc1ccc2c(c1)C(C)(C)c1cc(N(c3ccc(C45CC6CC(CC(C6)C4)C5)cc3)c3ccccc3-c3ccccc3-c3ccccc3-c3ccccc3)ccc1-2 ; Cc1ccc2c(c1)C(C)(C)c1cc(N(c3ccc(-c4ccccc4-c4ccc(C5CCCCC5)cc4)cc3)c3ccc(C45CC6CC(CC(C6)C4)C5)cc3)ccc1-2] 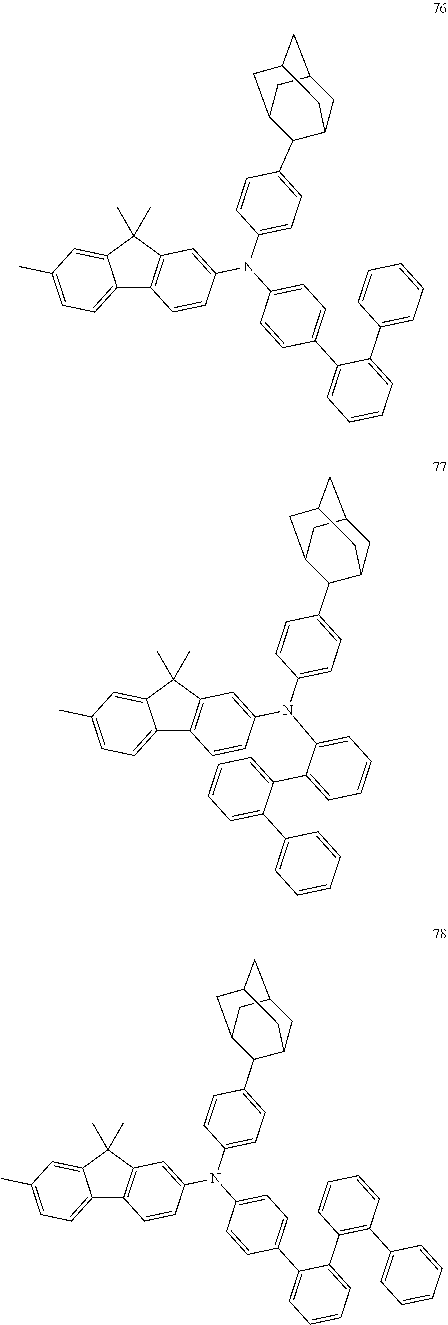 [SMILES: Cc1ccc2c(c1)C(C)(C)c1cc(N(c3ccc(-c4ccccc4-c4ccccc4)cc3)c3ccc(C4C5CC6CC(C5)CC4C6)cc3)ccc1-2 ; Cc1ccc2c(c1)C(C)(C)c1cc(N(c3ccc(C4C5CC6CC(C5)CC4C6)cc3)c3ccccc3-c3ccccc3-c3ccccc3)ccc1-2 ; Cc1ccc2c(c1)C(C)(C)c1cc(N(c3ccc(-c4ccccc4-c4ccccc4-c4ccccc4)cc3)c3ccc(C4C5CC6CC(C5)CC4C6)cc3)ccc1-2] 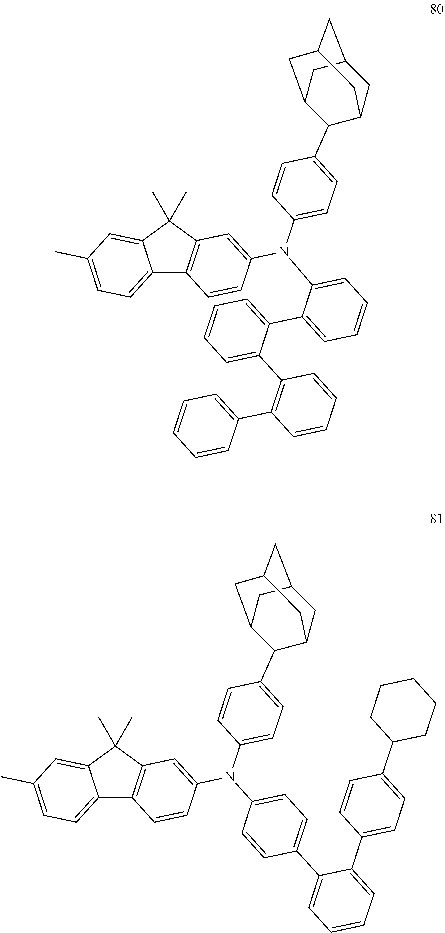 [SMILES: Cc1ccc2c(c1)C(C)(C)c1cc(N(c3ccc(C4C5CC6CC(C5)CC4C6)cc3)c3ccccc3-c3ccccc3-c3ccccc3-c3ccccc3)ccc1-2 ; Cc1ccc2c(c1)C(C)(C)c1cc(N(c3ccc(-c4ccccc4-c4ccc(C5CCCCC5)cc4)cc3)c3ccc(C4C5CC6CC(C5)CC4C6)cc3)ccc1-2] 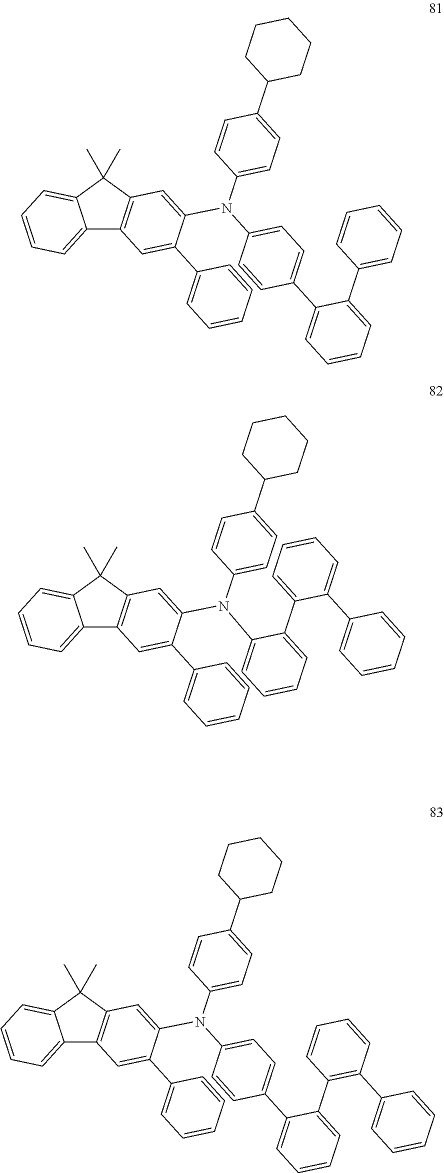 [SMILES: CC1(C)c2ccccc2-c2cc(-c3ccccc3)c(N(c3ccc(-c4ccccc4-c4ccccc4)cc3)c3ccc(C4CCCCC4)cc3)cc21 ; CC1(C)c2ccccc2-c2cc(-c3ccccc3)c(N(c3ccc(C4CCCCC4)cc3)c3ccccc3-c3ccccc3-c3ccccc3)cc21 ; CC1(C)c2ccccc2-c2cc(-c3ccccc3)c(N(c3ccc(-c4ccccc4-c4ccccc4-c4ccccc4)cc3)c3ccc(C4CCCCC4)cc3)cc21] 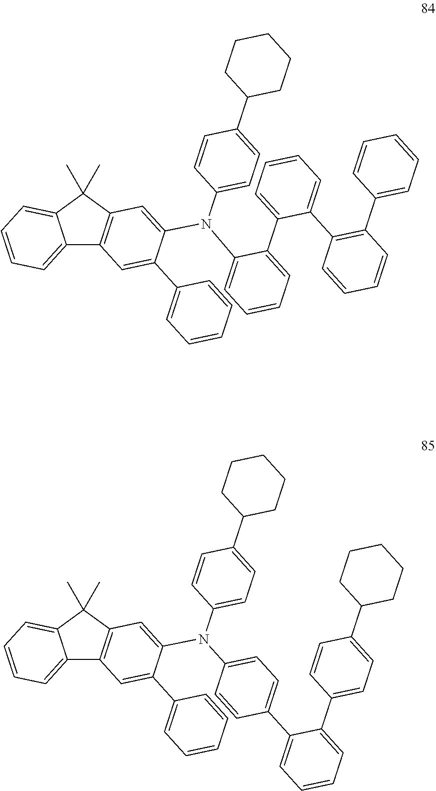 [SMILES: CC1(C)c2ccccc2-c2cc(-c3ccccc3)c(N(c3ccc(C4CCCCC4)cc3)c3ccccc3-c3ccccc3-c3ccccc3-c3ccccc3)cc21 ; CC1(C)c2ccccc2-c2cc(-c3ccccc3)c(N(c3ccc(-c4ccccc4-c4ccc(C5CCCCC5)cc4)cc3)c3ccc(C4CCCCC4)cc3)cc21] 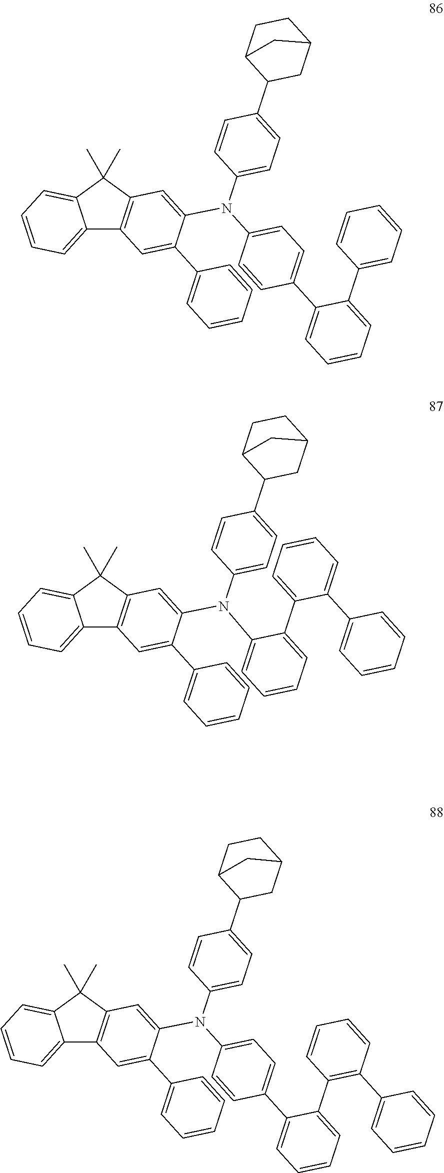 [SMILES: CC1(C)c2ccccc2-c2cc(-c3ccccc3)c(N(c3ccc(-c4ccccc4-c4ccccc4)cc3)c3ccc(C4CC5CCC4C5)cc3)cc21 ; CC1(C)c2ccccc2-c2cc(-c3ccccc3)c(N(c3ccc(C4CC5CCC4C5)cc3)c3ccccc3-c3ccccc3-c3ccccc3)cc21 ; CC1(C)c2ccccc2-c2cc(-c3ccccc3)c(N(c3ccc(-c4ccccc4-c4ccccc4-c4ccccc4)cc3)c3ccc(C4CC5CCC4C5)cc3)cc21] 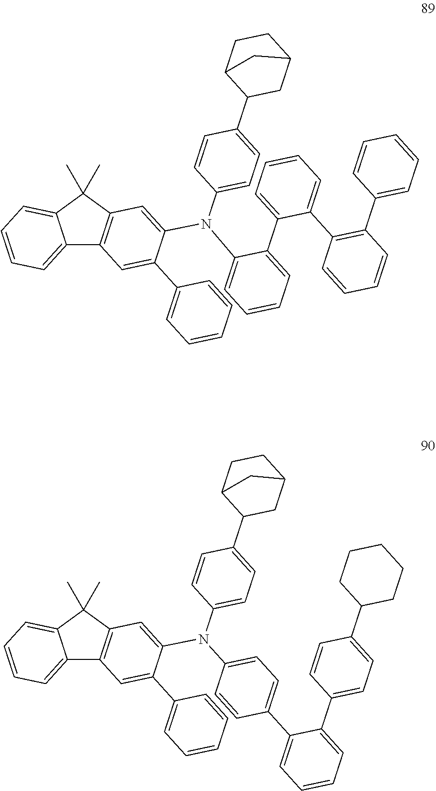 [SMILES: CC1(C)c2ccccc2-c2cc(-c3ccccc3)c(N(c3ccc(C4CC5CCC4C5)cc3)c3ccccc3-c3ccccc3-c3ccccc3-c3ccccc3)cc21 ; CC1(C)c2ccccc2-c2cc(-c3ccccc3)c(N(c3ccc(-c4ccccc4-c4ccc(C5CCCCC5)cc4)cc3)c3ccc(C4CC5CCC4C5)cc3)cc21] 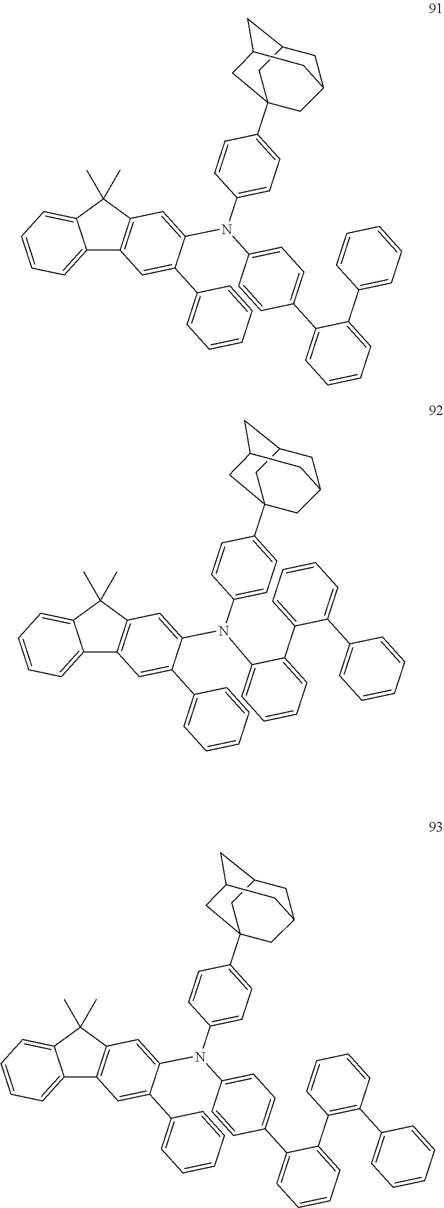 [SMILES: CC1(C)c2ccccc2-c2cc(-c3ccccc3)c(N(c3ccc(-c4ccccc4-c4ccccc4)cc3)c3ccc(C45CC6CC(CC(C6)C4)C5)cc3)cc21 ; CC1(C)c2ccccc2-c2cc(-c3ccccc3)c(N(c3ccc(C45CC6CC(CC(C6)C4)C5)cc3)c3ccccc3-c3ccccc3-c3ccccc3)cc21 ; CC1(C)c2ccccc2-c2cc(-c3ccccc3)c(N(c3ccc(-c4ccccc4-c4ccccc4-c4ccccc4)cc3)c3ccc(C45CC6CC(CC(C6)C4)C5)cc3)cc21] 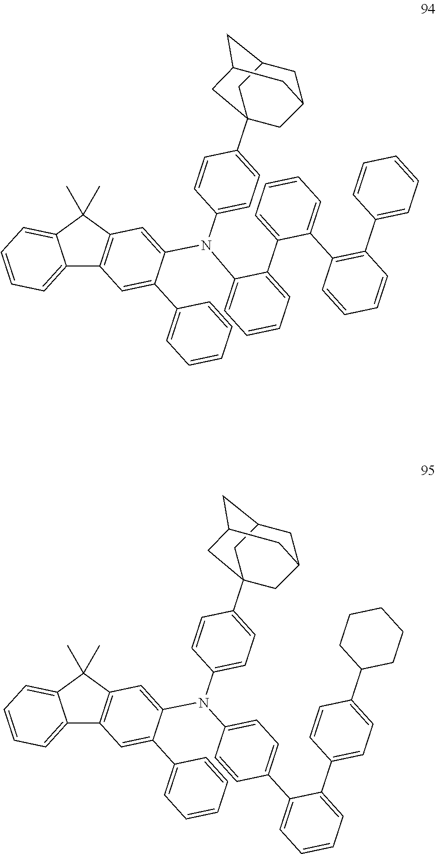 [SMILES: CC1(C)c2ccccc2-c2cc(-c3ccccc3)c(N(c3ccc(C45CC6CC(CC(C6)C4)C5)cc3)c3ccccc3-c3ccccc3-c3ccccc3-c3ccccc3)cc21 ; CC1(C)c2ccccc2-c2cc(-c3ccccc3)c(N(c3ccc(-c4ccccc4-c4ccc(C5CCCCC5)cc4)cc3)c3ccc(C45CC6CC(CC(C6)C4)C5)cc3)cc21] 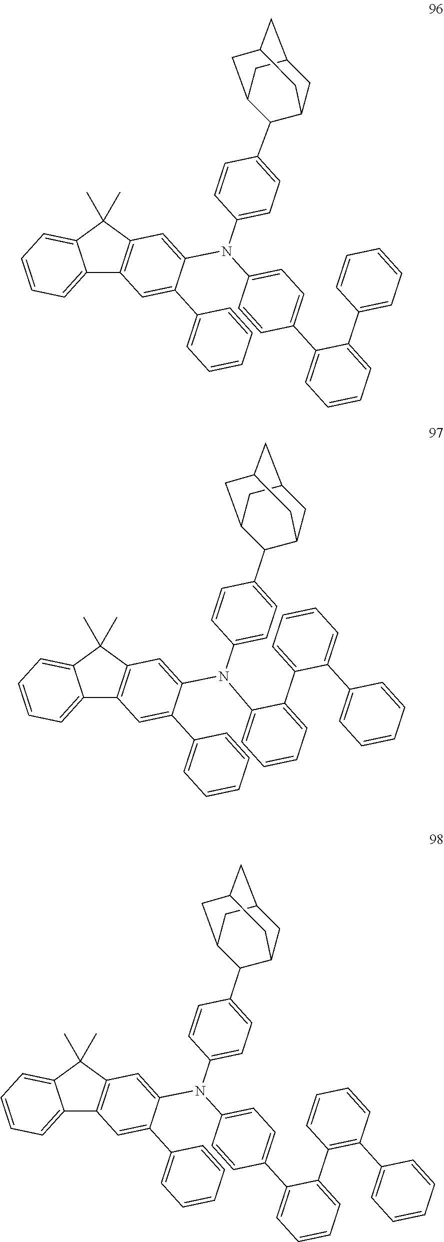 [SMILES: CC1(C)c2ccccc2-c2cc(-c3ccccc3)c(N(c3ccc(-c4ccccc4-c4ccccc4)cc3)c3ccc(C4C5CC6CC(C5)CC4C6)cc3)cc21 ; CC1(C)c2ccccc2-c2cc(-c3ccccc3)c(N(c3ccc(C4C5CC6CC(C5)CC4C6)cc3)c3ccccc3-c3ccccc3-c3ccccc3)cc21 ; CC1(C)c2ccccc2-c2cc(-c3ccccc3)c(N(c3ccc(-c4ccccc4-c4ccccc4-c4ccccc4)cc3)c3ccc(C4C5CC6CC(C5)CC4C6)cc3)cc21] 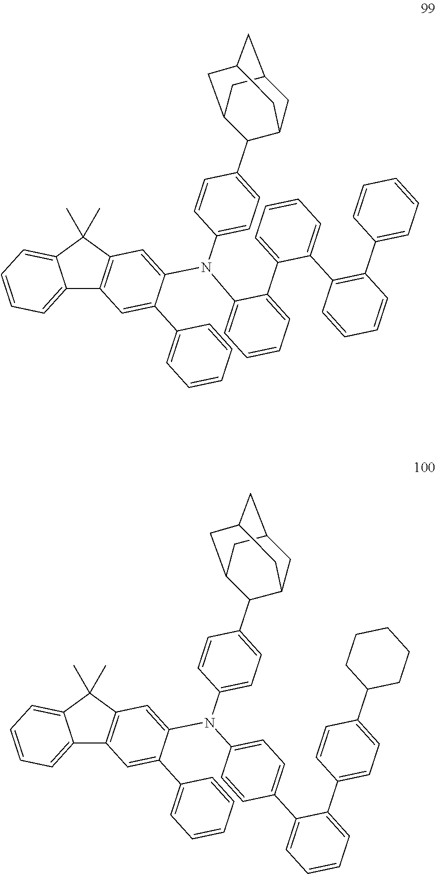 [SMILES: CC1(C)c2ccccc2-c2cc(-c3ccccc3)c(N(c3ccc(C4C5CC6CC(C5)CC4C6)cc3)c3ccccc3-c3ccccc3-c3ccccc3-c3ccccc3)cc21 ; CC1(C)c2ccccc2-c2cc(-c3ccccc3)c(N(c3ccc(-c4ccccc4-c4ccc(C5CCCCC5)cc4)cc3)c3ccc(C4C5CC6CC(C5)CC4C6)cc3)cc21] 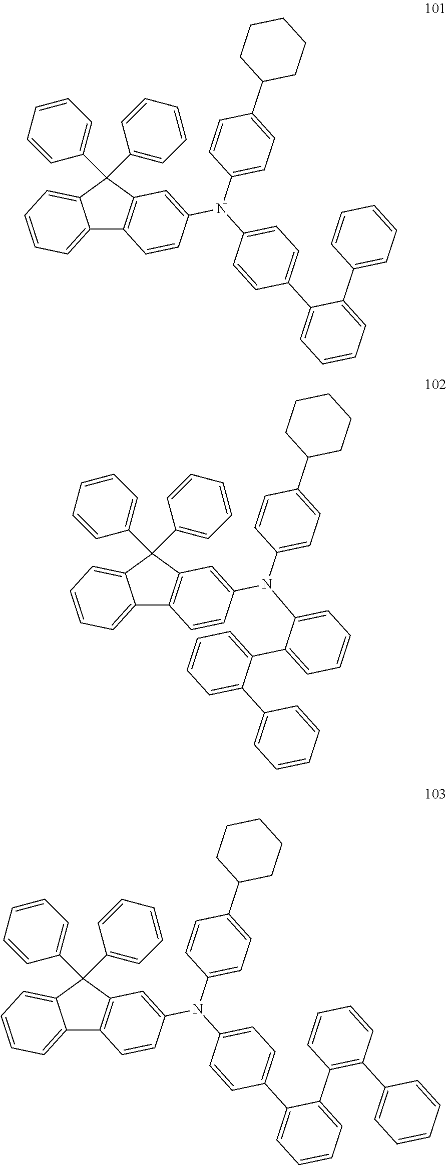 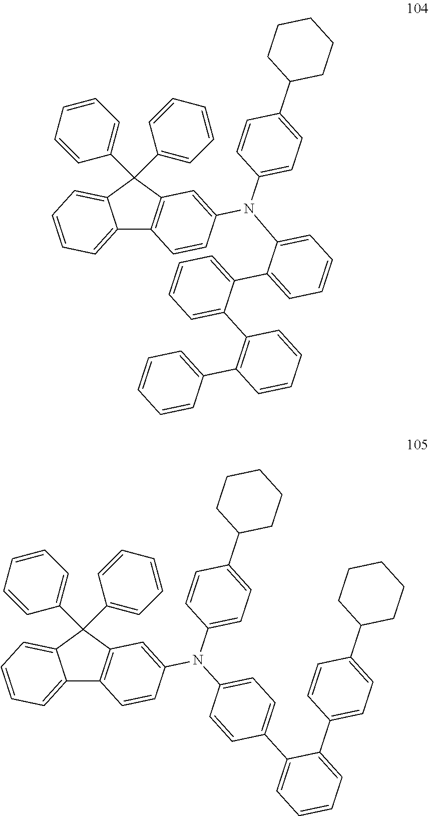 [SMILES: c1ccc(-c2ccccc2-c2ccccc2-c2ccccc2N(c2ccc(C3CCCCC3)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccccc5-c5ccc(C6CCCCC6)cc5)cc4)c4ccc(C5CCCCC5)cc4)cc32)cc1] 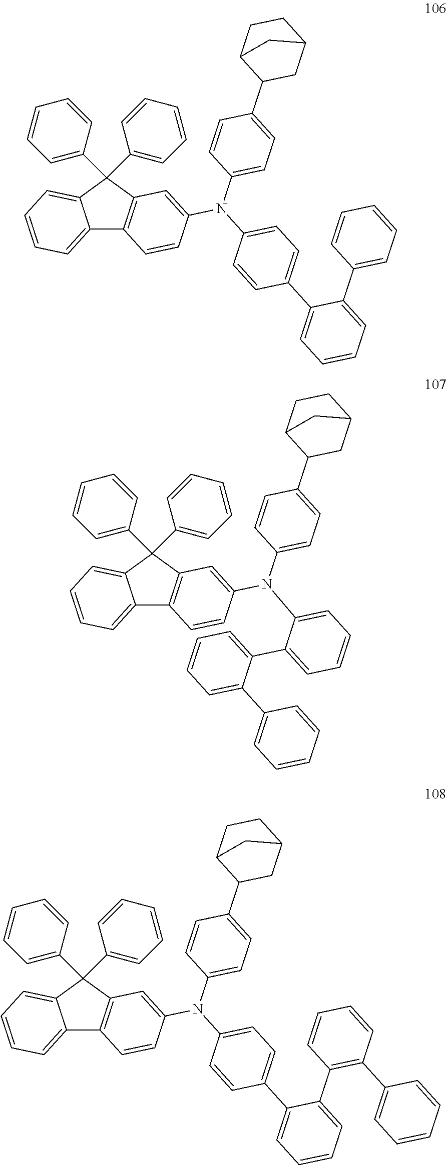 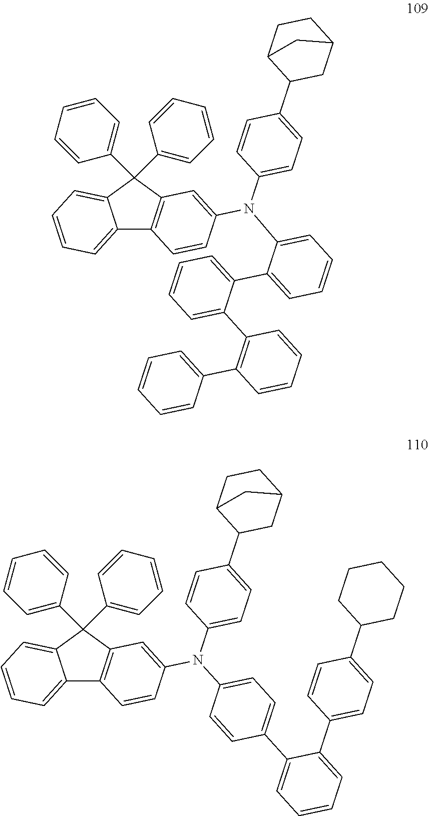 [SMILES: c1ccc(-c2ccccc2-c2ccccc2-c2ccccc2N(c2ccc(C3CC4CCC3C4)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccccc5-c5ccc(C6CCCCC6)cc5)cc4)c4ccc(C5CC6CCC5C6)cc4)cc32)cc1] 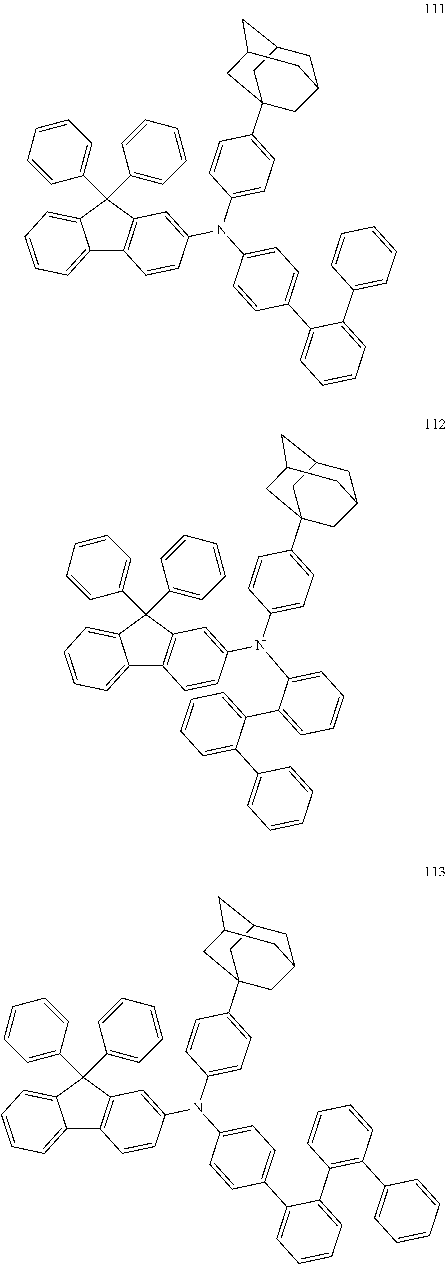 [SMILES: c1ccc(-c2ccccc2-c2ccc(N(c3ccc(C45CC6CC(CC(C6)C4)C5)cc3)c3ccc4c(c3)C(c3ccccc3)(c3ccccc3)c3ccccc3-4)cc2)cc1 ; c1ccc(-c2ccccc2-c2ccccc2N(c2ccc(C34CC5CC(CC(C5)C3)C4)cc2)c2ccc3c(c2)C(c2ccccc2)(c2ccccc2)c2ccccc2-3)cc1 ; c1ccc(-c2ccccc2-c2ccccc2-c2ccc(N(c3ccc(C45CC6CC(CC(C6)C4)C5)cc3)c3ccc4c(c3)C(c3ccccc3)(c3ccccc3)c3ccccc3-4)cc2)cc1] 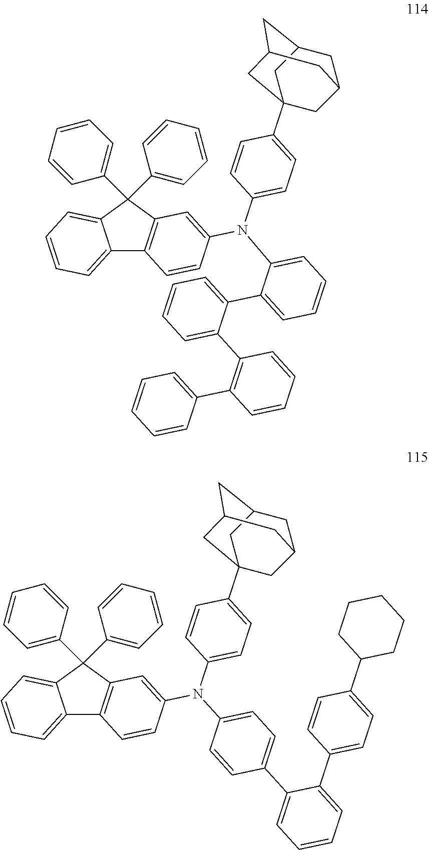 [SMILES: c1ccc(-c2ccccc2-c2ccccc2-c2ccccc2N(c2ccc(C34CC5CC(CC(C5)C3)C4)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccccc5-c5ccc(C6CCCCC6)cc5)cc4)c4ccc(C56CC7CC(CC(C7)C5)C6)cc4)cc32)cc1] 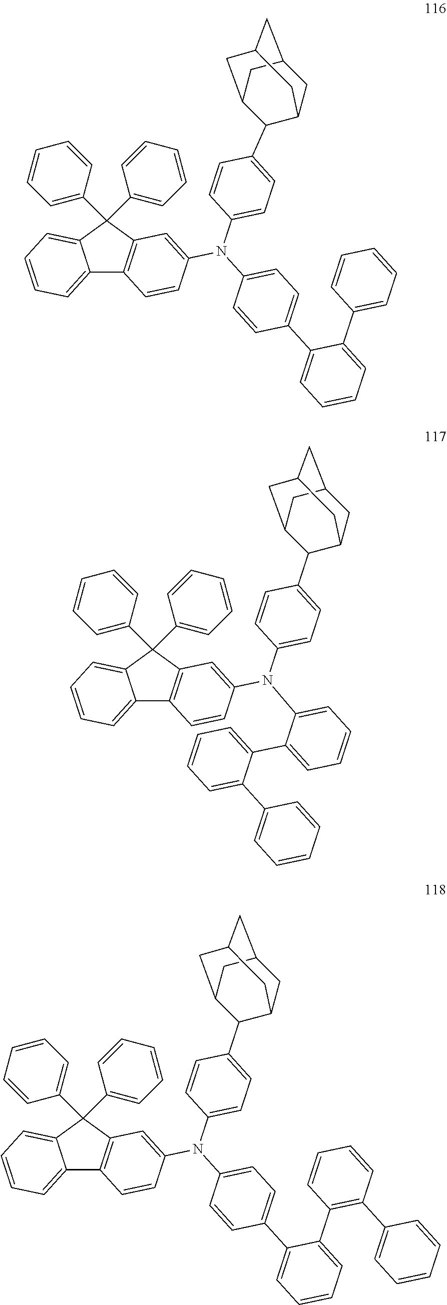 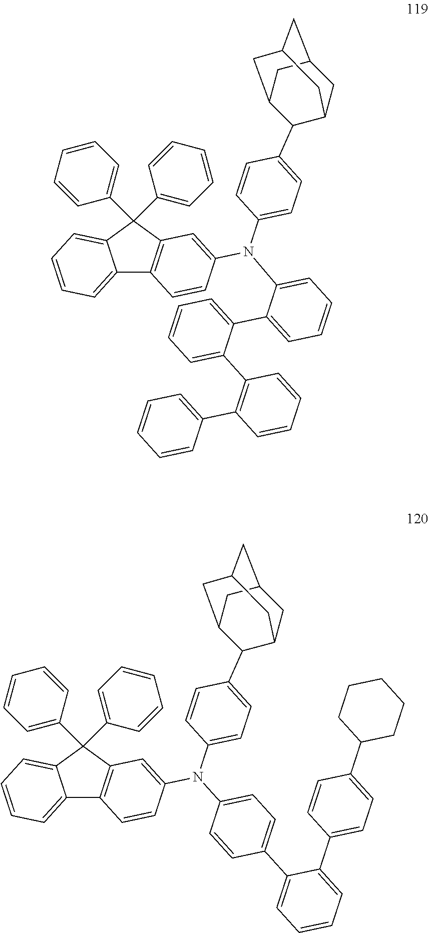 [SMILES: c1ccc(-c2ccccc2-c2ccccc2-c2ccccc2N(c2ccc(C3C4CC5CC(C4)CC3C5)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccccc5-c5ccc(C6CCCCC6)cc5)cc4)c4ccc(C5C6CC7CC(C6)CC5C7)cc4)cc32)cc1] 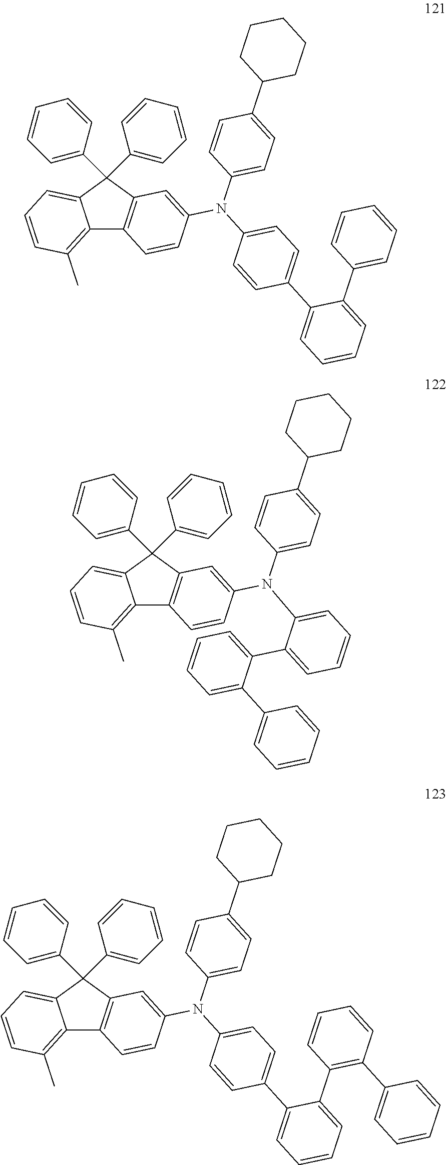 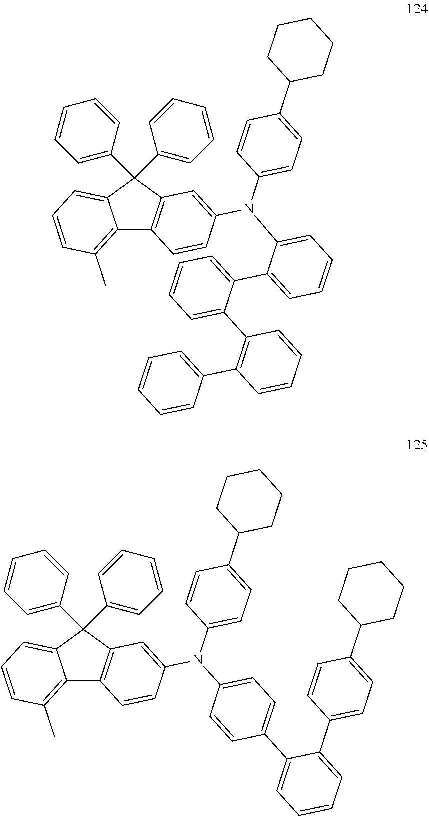 [SMILES: Cc1cccc2c1-c1ccc(N(c3ccc(C4CCCCC4)cc3)c3ccccc3-c3ccccc3-c3ccccc3-c3ccccc3)cc1C2(c1ccccc1)c1ccccc1 ; Cc1cccc2c1-c1ccc(N(c3ccc(-c4ccccc4-c4ccc(C5CCCCC5)cc4)cc3)c3ccc(C4CCCCC4)cc3)cc1C2(c1ccccc1)c1ccccc1] 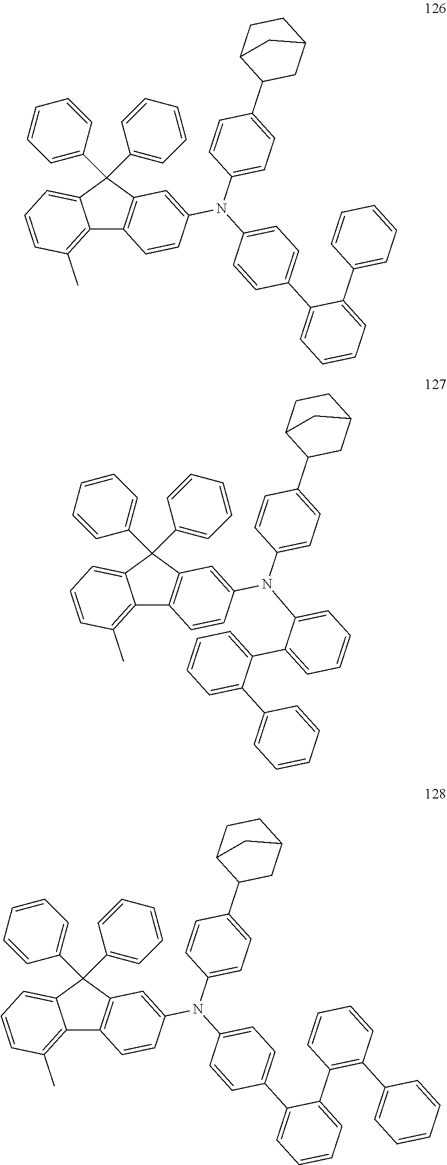 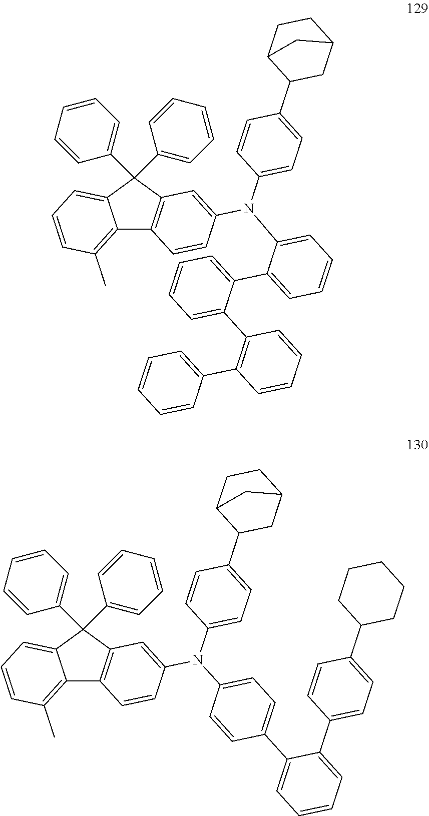 [SMILES: Cc1cccc2c1-c1ccc(N(c3ccc(C4CC5CCC4C5)cc3)c3ccccc3-c3ccccc3-c3ccccc3-c3ccccc3)cc1C2(c1ccccc1)c1ccccc1 ; Cc1cccc2c1-c1ccc(N(c3ccc(-c4ccccc4-c4ccc(C5CCCCC5)cc4)cc3)c3ccc(C4CC5CCC4C5)cc3)cc1C2(c1ccccc1)c1ccccc1] 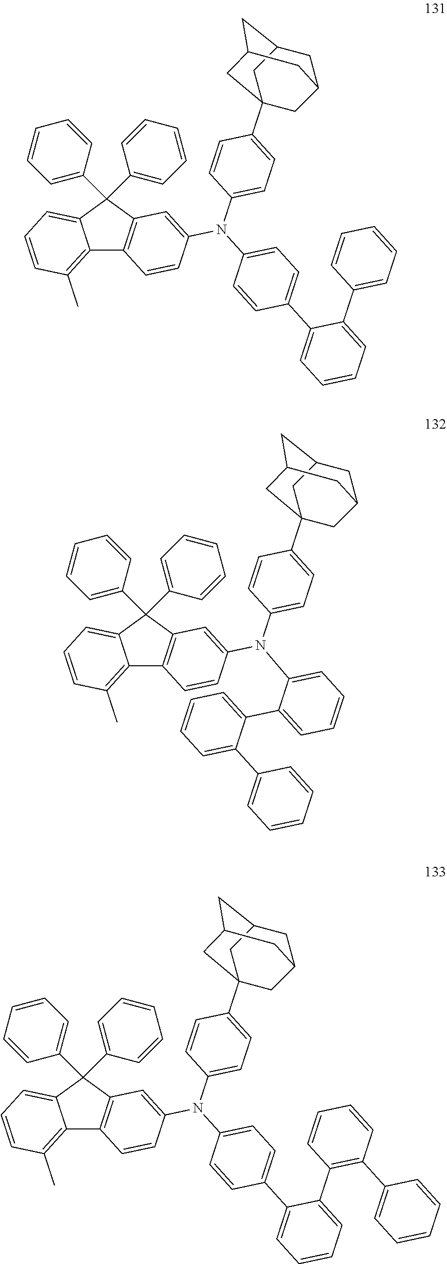 [SMILES: Cc1cccc2c1-c1ccc(N(c3ccc(-c4ccccc4-c4ccccc4)cc3)c3ccc(C45CC6CC(CC(C6)C4)C5)cc3)cc1C2(c1ccccc1)c1ccccc1 ; Cc1cccc2c1-c1ccc(N(c3ccc(C45CC6CC(CC(C6)C4)C5)cc3)c3ccccc3-c3ccccc3-c3ccccc3)cc1C2(c1ccccc1)c1ccccc1 ; Cc1cccc2c1-c1ccc(N(c3ccc(-c4ccccc4-c4ccccc4-c4ccccc4)cc3)c3ccc(C45CC6CC(CC(C6)C4)C5)cc3)cc1C2(c1ccccc1)c1ccccc1] 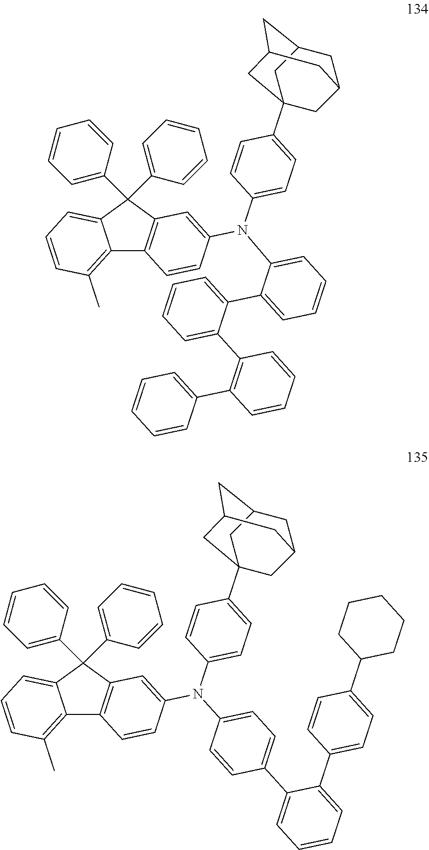 [SMILES: Cc1cccc2c1-c1ccc(N(c3ccc(C45CC6CC(CC(C6)C4)C5)cc3)c3ccccc3-c3ccccc3-c3ccccc3-c3ccccc3)cc1C2(c1ccccc1)c1ccccc1 ; Cc1cccc2c1-c1ccc(N(c3ccc(-c4ccccc4-c4ccc(C5CCCCC5)cc4)cc3)c3ccc(C45CC6CC(CC(C6)C4)C5)cc3)cc1C2(c1ccccc1)c1ccccc1] 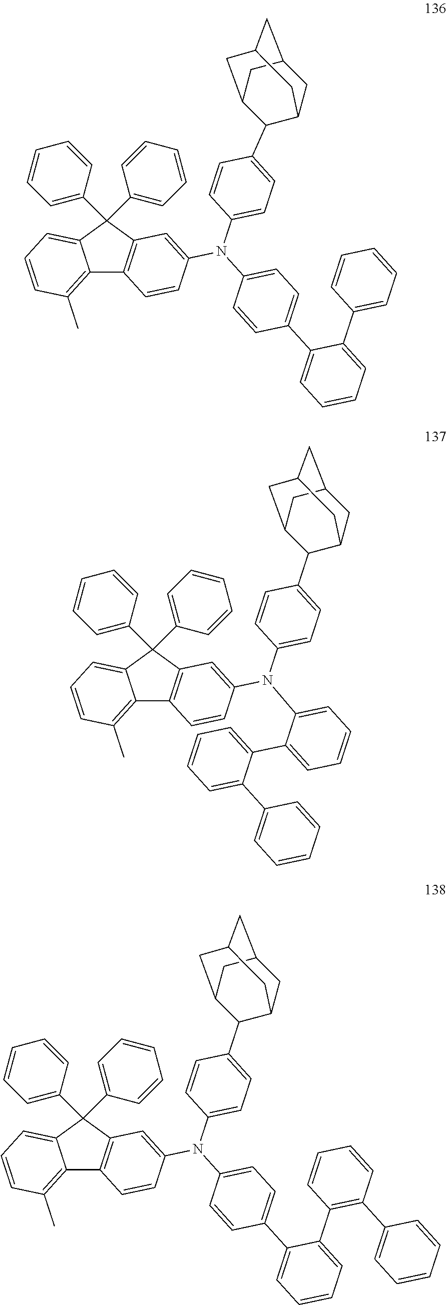 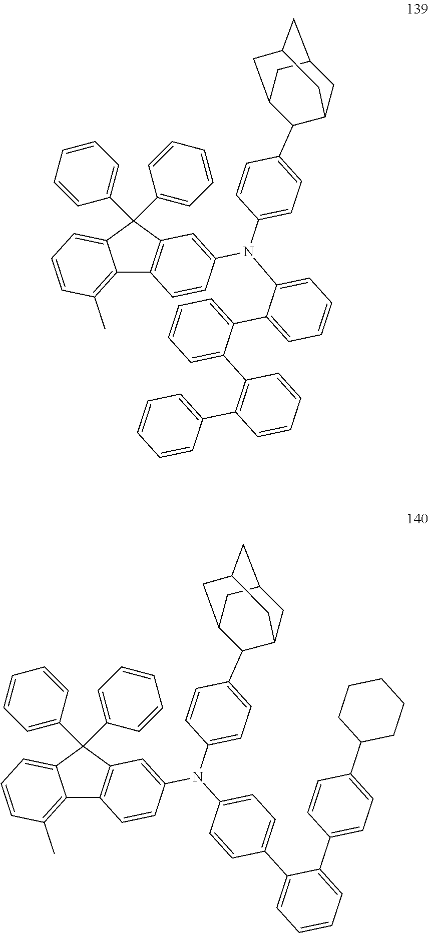 [SMILES: Cc1cccc2c1-c1ccc(N(c3ccc(C4C5CC6CC(C5)CC4C6)cc3)c3ccccc3-c3ccccc3-c3ccccc3-c3ccccc3)cc1C2(c1ccccc1)c1ccccc1 ; Cc1cccc2c1-c1ccc(N(c3ccc(-c4ccccc4-c4ccc(C5CCCCC5)cc4)cc3)c3ccc(C4C5CC6CC(C5)CC4C6)cc3)cc1C2(c1ccccc1)c1ccccc1] 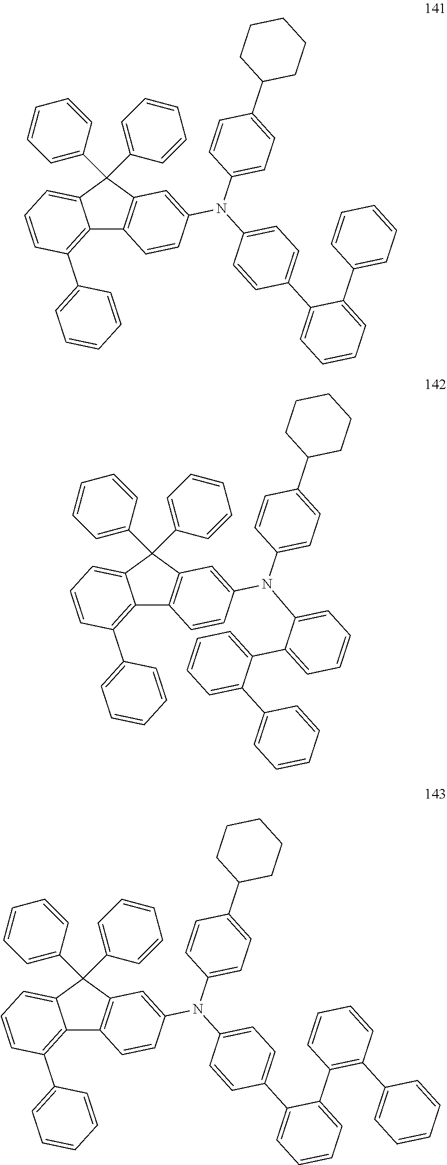 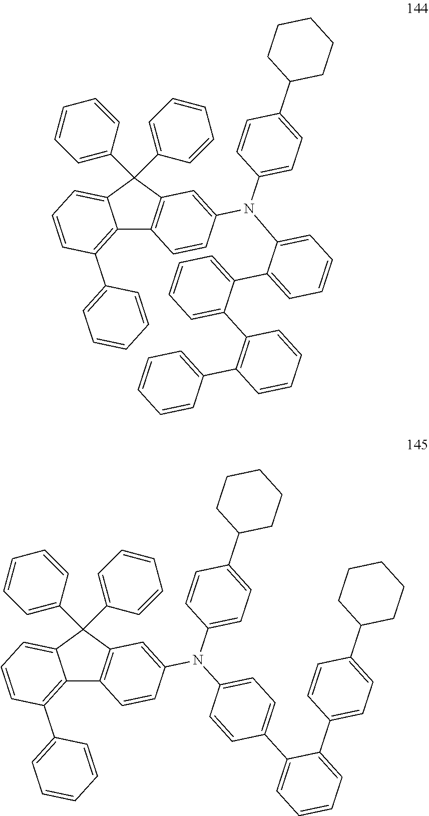 [SMILES: c1ccc(-c2ccccc2-c2ccccc2-c2ccccc2N(c2ccc(C3CCCCC3)cc2)c2ccc3c(c2)C(c2ccccc2)(c2ccccc2)c2cccc(-c4ccccc4)c2-3)cc1 ; c1ccc(-c2cccc3c2-c2ccc(N(c4ccc(-c5ccccc5-c5ccc(C6CCCCC6)cc5)cc4)c4ccc(C5CCCCC5)cc4)cc2C3(c2ccccc2)c2ccccc2)cc1] 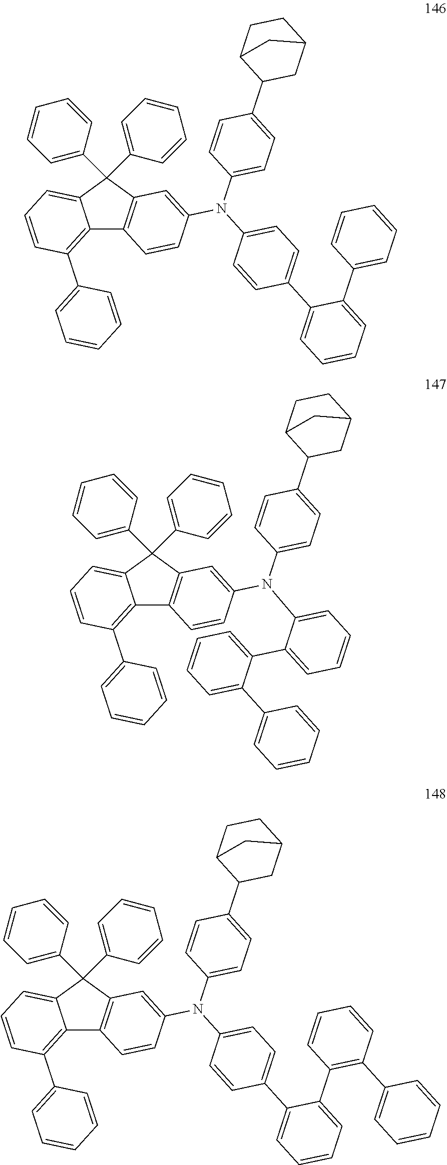 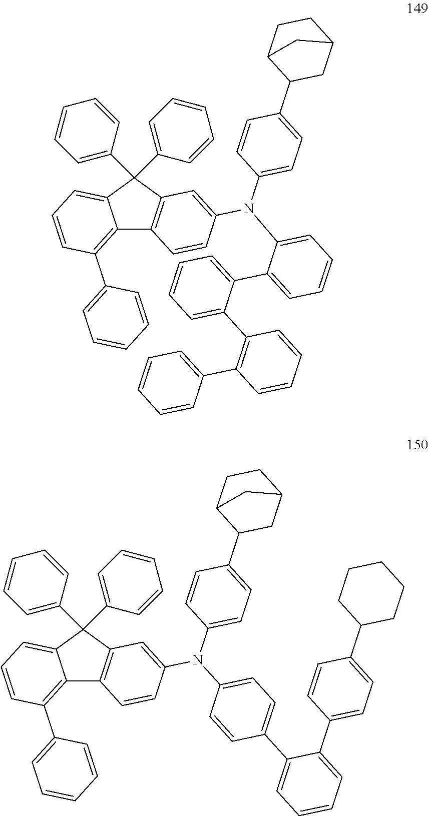 [SMILES: c1ccc(-c2ccccc2-c2ccccc2-c2ccccc2N(c2ccc(C3CC4CCC3C4)cc2)c2ccc3c(c2)C(c2ccccc2)(c2ccccc2)c2cccc(-c4ccccc4)c2-3)cc1 ; c1ccc(-c2cccc3c2-c2ccc(N(c4ccc(-c5ccccc5-c5ccc(C6CCCCC6)cc5)cc4)c4ccc(C5CC6CCC5C6)cc4)cc2C3(c2ccccc2)c2ccccc2)cc1] 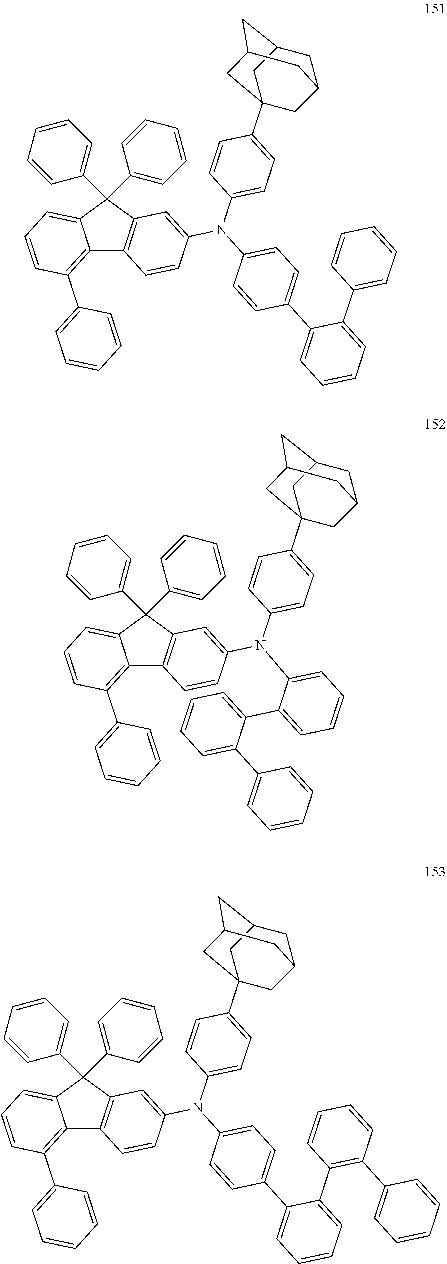 [SMILES: c1ccc(-c2ccccc2-c2ccc(N(c3ccc(C45CC6CC(CC(C6)C4)C5)cc3)c3ccc4c(c3)C(c3ccccc3)(c3ccccc3)c3cccc(-c5ccccc5)c3-4)cc2)cc1 ; c1ccc(-c2ccccc2-c2ccccc2N(c2ccc(C34CC5CC(CC(C5)C3)C4)cc2)c2ccc3c(c2)C(c2ccccc2)(c2ccccc2)c2cccc(-c4ccccc4)c2-3)cc1 ; c1ccc(-c2ccccc2-c2ccccc2-c2ccc(N(c3ccc(C45CC6CC(CC(C6)C4)C5)cc3)c3ccc4c(c3)C(c3ccccc3)(c3ccccc3)c3cccc(-c5ccccc5)c3-4)cc2)cc1] 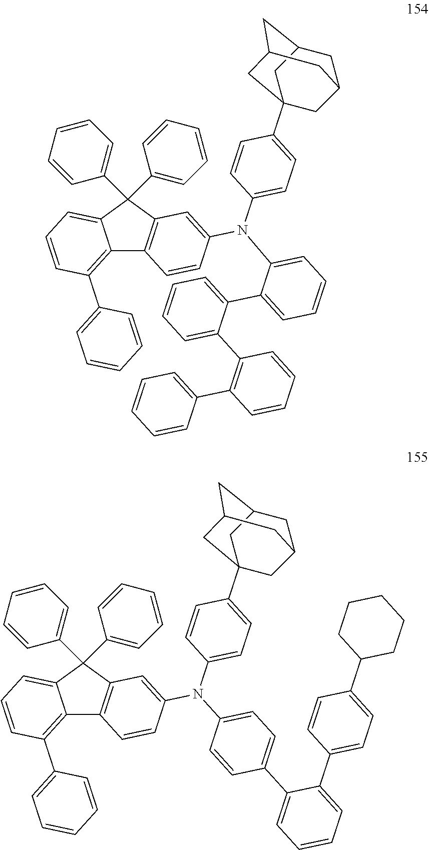 [SMILES: c1ccc(-c2ccccc2-c2ccccc2-c2ccccc2N(c2ccc(C34CC5CC(CC(C5)C3)C4)cc2)c2ccc3c(c2)C(c2ccccc2)(c2ccccc2)c2cccc(-c4ccccc4)c2-3)cc1 ; c1ccc(-c2cccc3c2-c2ccc(N(c4ccc(-c5ccccc5-c5ccc(C6CCCCC6)cc5)cc4)c4ccc(C56CC7CC(CC(C7)C5)C6)cc4)cc2C3(c2ccccc2)c2ccccc2)cc1] 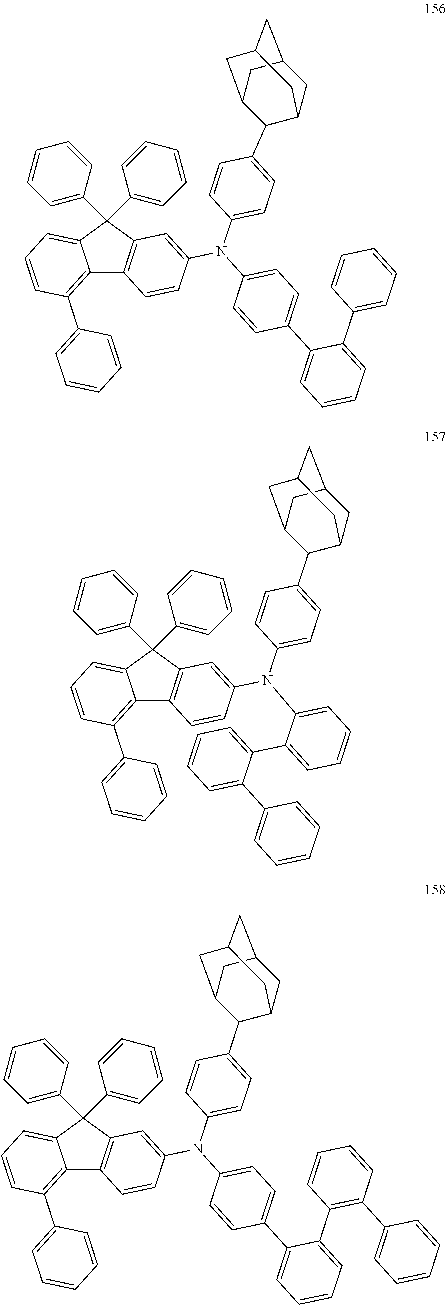 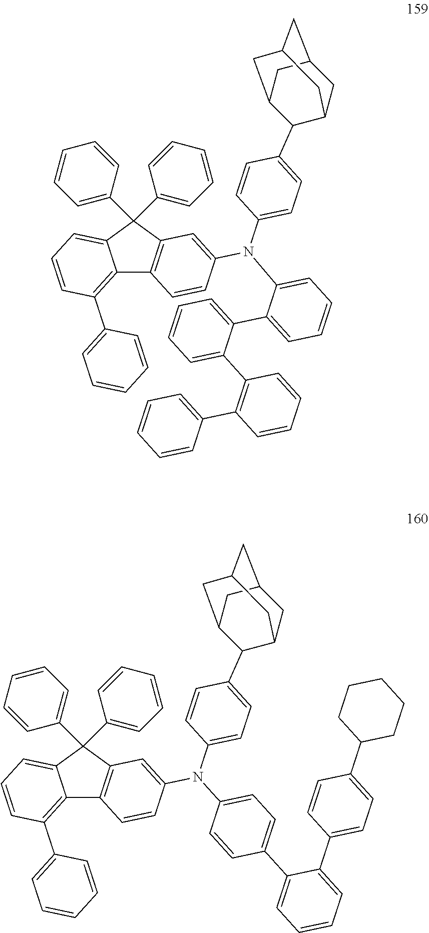 [SMILES: c1ccc(-c2ccccc2-c2ccccc2-c2ccccc2N(c2ccc(C3C4CC5CC(C4)CC3C5)cc2)c2ccc3c(c2)C(c2ccccc2)(c2ccccc2)c2cccc(-c4ccccc4)c2-3)cc1 ; c1ccc(-c2cccc3c2-c2ccc(N(c4ccc(-c5ccccc5-c5ccc(C6CCCCC6)cc5)cc4)c4ccc(C5C6CC7CC(C6)CC5C7)cc4)cc2C3(c2ccccc2)c2ccccc2)cc1] 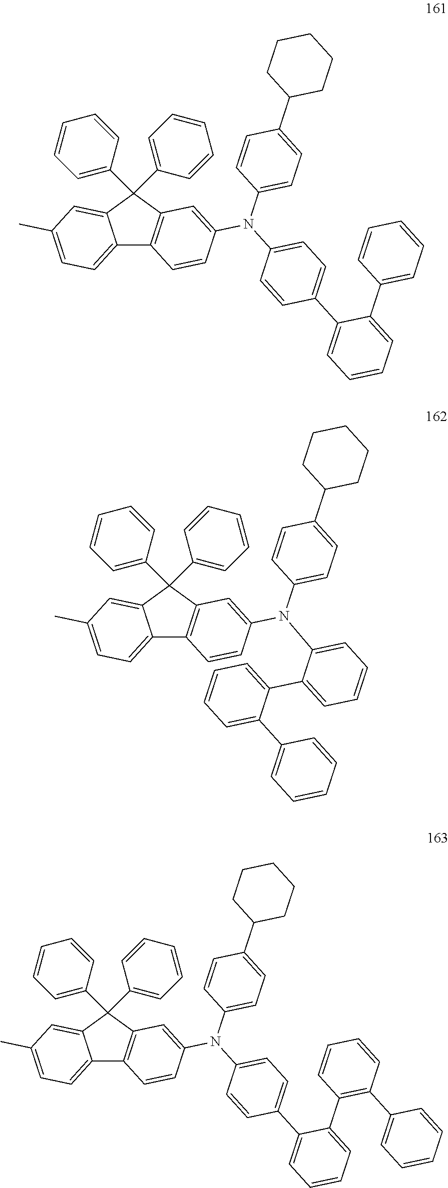 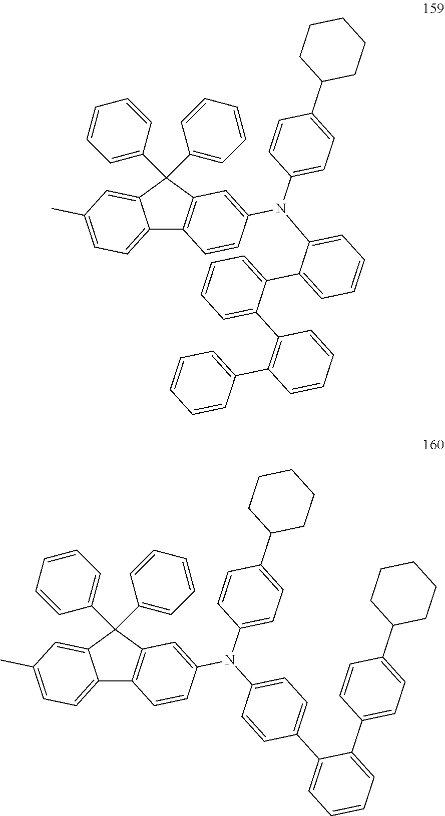 [SMILES: Cc1ccc2c(c1)C(c1ccccc1)(c1ccccc1)c1cc(N(c3ccc(C4CCCCC4)cc3)c3ccccc3-c3ccccc3-c3ccccc3-c3ccccc3)ccc1-2 ; Cc1ccc2c(c1)C(c1ccccc1)(c1ccccc1)c1cc(N(c3ccc(-c4ccccc4-c4ccc(C5CCCCC5)cc4)cc3)c3ccc(C4CCCCC4)cc3)ccc1-2] 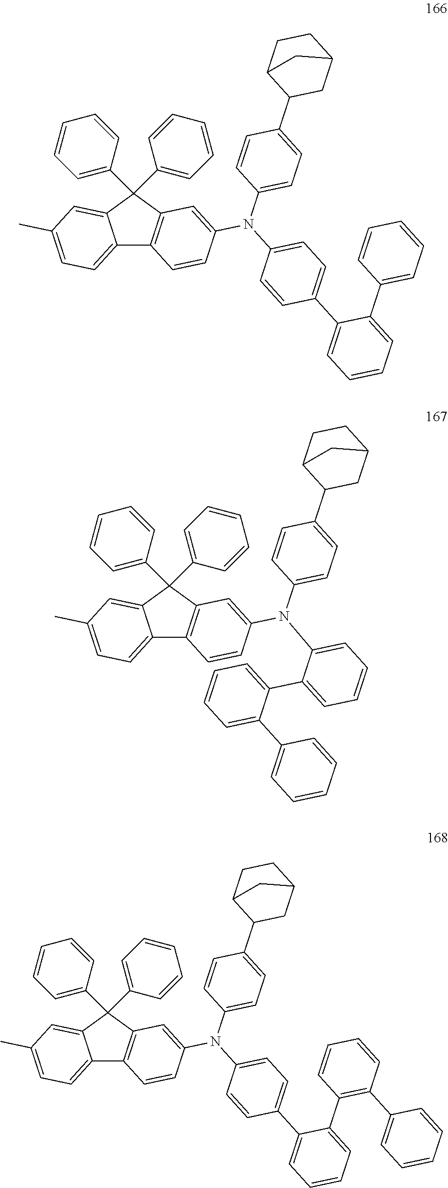 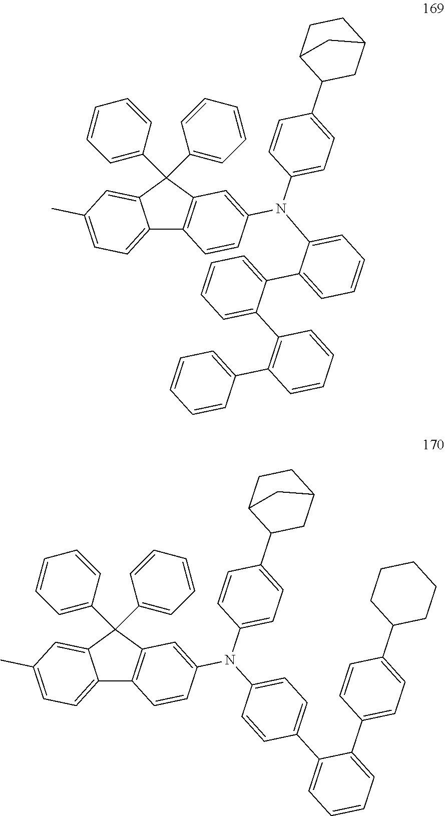 [SMILES: Cc1ccc2c(c1)C(c1ccccc1)(c1ccccc1)c1cc(N(c3ccc(C4CC5CCC4C5)cc3)c3ccccc3-c3ccccc3-c3ccccc3-c3ccccc3)ccc1-2 ; Cc1ccc2c(c1)C(c1ccccc1)(c1ccccc1)c1cc(N(c3ccc(-c4ccccc4-c4ccc(C5CCCCC5)cc4)cc3)c3ccc(C4CC5CCC4C5)cc3)ccc1-2] 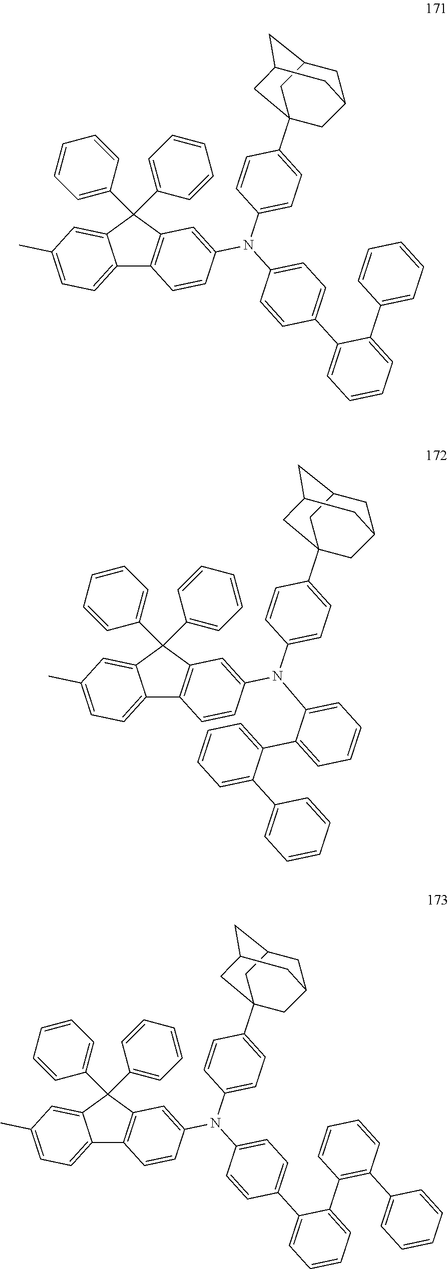 [SMILES: Cc1ccc2c(c1)C(c1ccccc1)(c1ccccc1)c1cc(N(c3ccc(-c4ccccc4-c4ccccc4)cc3)c3ccc(C45CC6CC(CC(C6)C4)C5)cc3)ccc1-2 ; Cc1ccc2c(c1)C(c1ccccc1)(c1ccccc1)c1cc(N(c3ccc(C45CC6CC(CC(C6)C4)C5)cc3)c3ccccc3-c3ccccc3-c3ccccc3)ccc1-2 ; Cc1ccc2c(c1)C(c1ccccc1)(c1ccccc1)c1cc(N(c3ccc(-c4ccccc4-c4ccccc4-c4ccccc4)cc3)c3ccc(C45CC6CC(CC(C6)C4)C5)cc3)ccc1-2] 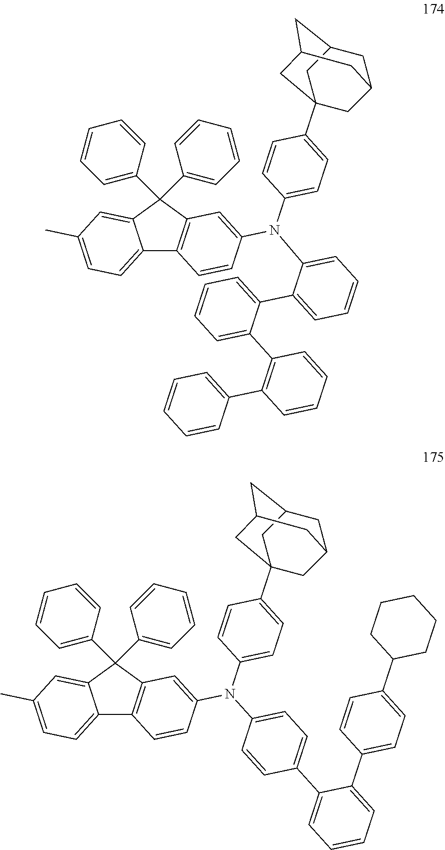 [SMILES: Cc1ccc2c(c1)C(c1ccccc1)(c1ccccc1)c1cc(N(c3ccc(C45CC6CC(CC(C6)C4)C5)cc3)c3ccccc3-c3ccccc3-c3ccccc3-c3ccccc3)ccc1-2 ; Cc1ccc2c(c1)C(c1ccccc1)(c1ccccc1)c1cc(N(c3ccc(-c4ccccc4-c4ccc(C5CCCCC5)cc4)cc3)c3ccc(C45CC6CC(CC(C6)C4)C5)cc3)ccc1-2] 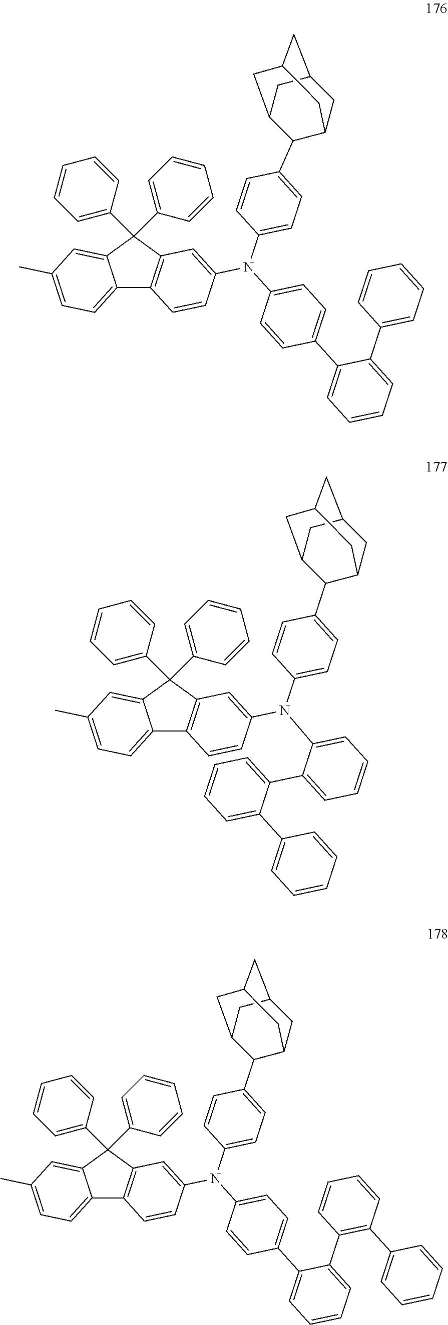 [SMILES: Cc1ccc2c(c1)C(c1ccccc1)(c1ccccc1)c1cc(N(c3ccc(-c4ccccc4-c4ccccc4)cc3)c3ccc(C4C5CC6CC(C5)CC4C6)cc3)ccc1-2 ; Cc1ccc2c(c1)C(c1ccccc1)(c1ccccc1)c1cc(N(c3ccc(C4C5CC6CC(C5)CC4C6)cc3)c3ccccc3-c3ccccc3-c3ccccc3)ccc1-2 ; Cc1ccc2c(c1)C(c1ccccc1)(c1ccccc1)c1cc(N(c3ccc(-c4ccccc4-c4ccccc4-c4ccccc4)cc3)c3ccc(C4C5CC6CC(C5)CC4C6)cc3)ccc1-2] 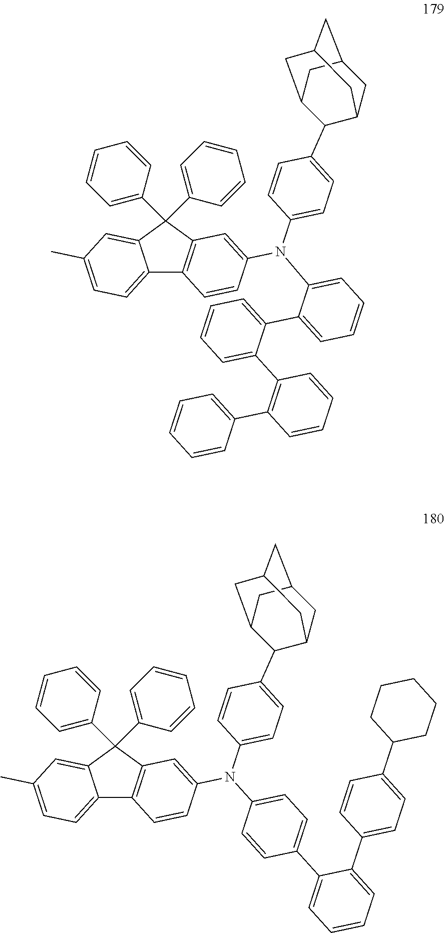 [SMILES: Cc1ccc2c(c1)C(c1ccccc1)(c1ccccc1)c1cc(N(c3ccc(C4C5CC6CC(C5)CC4C6)cc3)c3ccccc3-c3ccccc3-c3ccccc3-c3ccccc3)ccc1-2 ; Cc1ccc2c(c1)C(c1ccccc1)(c1ccccc1)c1cc(N(c3ccc(-c4ccccc4-c4ccc(C5CCCCC5)cc4)cc3)c3ccc(C4C5CC6CC(C5)CC4C6)cc3)ccc1-2] 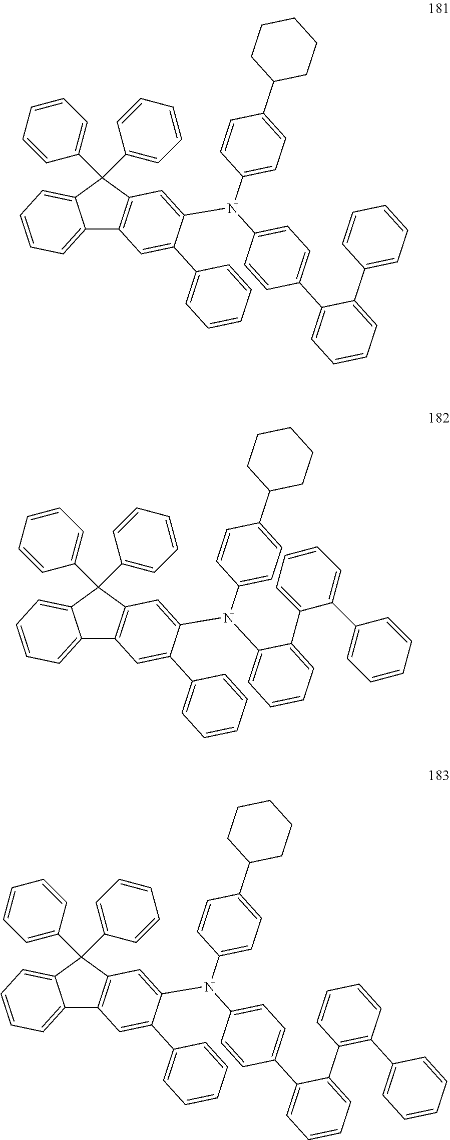 [SMILES: c1ccc(-c2ccccc2-c2ccc(N(c3ccc(C4CCCCC4)cc3)c3cc4c(cc3-c3ccccc3)-c3ccccc3C4(c3ccccc3)c3ccccc3)cc2)cc1 ; c1ccc(-c2ccccc2-c2ccccc2N(c2ccc(C3CCCCC3)cc2)c2cc3c(cc2-c2ccccc2)-c2ccccc2C3(c2ccccc2)c2ccccc2)cc1 ; c1ccc(-c2ccccc2-c2ccccc2-c2ccc(N(c3ccc(C4CCCCC4)cc3)c3cc4c(cc3-c3ccccc3)-c3ccccc3C4(c3ccccc3)c3ccccc3)cc2)cc1] 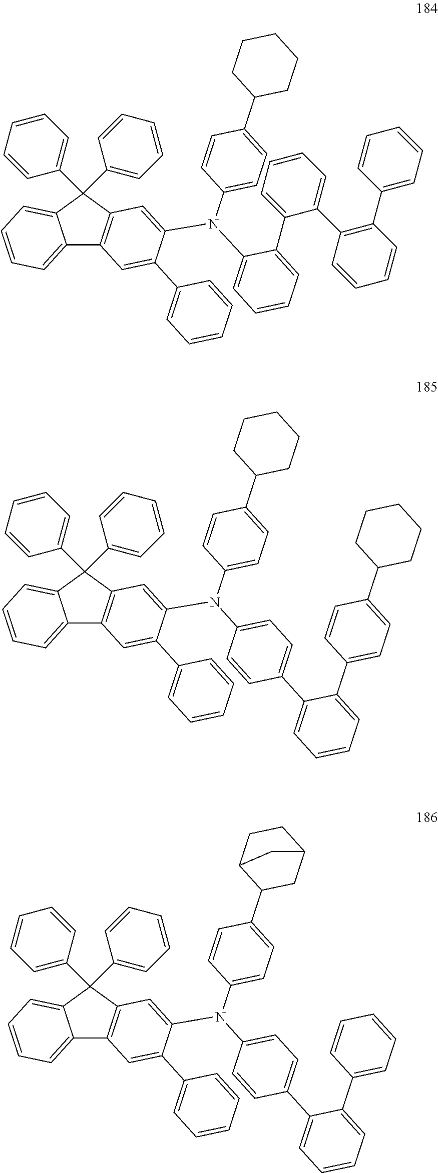 [SMILES: c1ccc(-c2ccccc2-c2ccccc2-c2ccccc2N(c2ccc(C3CCCCC3)cc2)c2cc3c(cc2-c2ccccc2)-c2ccccc2C3(c2ccccc2)c2ccccc2)cc1 ; c1ccc(-c2cc3c(cc2N(c2ccc(-c4ccccc4-c4ccc(C5CCCCC5)cc4)cc2)c2ccc(C4CCCCC4)cc2)C(c2ccccc2)(c2ccccc2)c2ccccc2-3)cc1 ; c1ccc(-c2ccccc2-c2ccc(N(c3ccc(C4CC5CCC4C5)cc3)c3cc4c(cc3-c3ccccc3)-c3ccccc3C4(c3ccccc3)c3ccccc3)cc2)cc1] 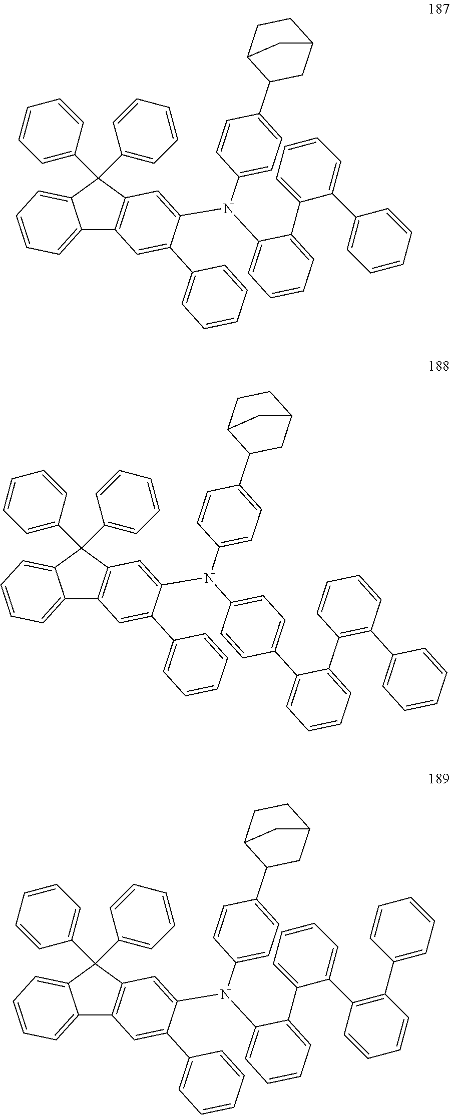 [SMILES: c1ccc(-c2ccccc2-c2ccccc2N(c2ccc(C3CC4CCC3C4)cc2)c2cc3c(cc2-c2ccccc2)-c2ccccc2C3(c2ccccc2)c2ccccc2)cc1 ; c1ccc(-c2ccccc2-c2ccccc2-c2ccc(N(c3ccc(C4CC5CCC4C5)cc3)c3cc4c(cc3-c3ccccc3)-c3ccccc3C4(c3ccccc3)c3ccccc3)cc2)cc1 ; c1ccc(-c2ccccc2-c2ccccc2-c2ccccc2N(c2ccc(C3CC4CCC3C4)cc2)c2cc3c(cc2-c2ccccc2)-c2ccccc2C3(c2ccccc2)c2ccccc2)cc1] 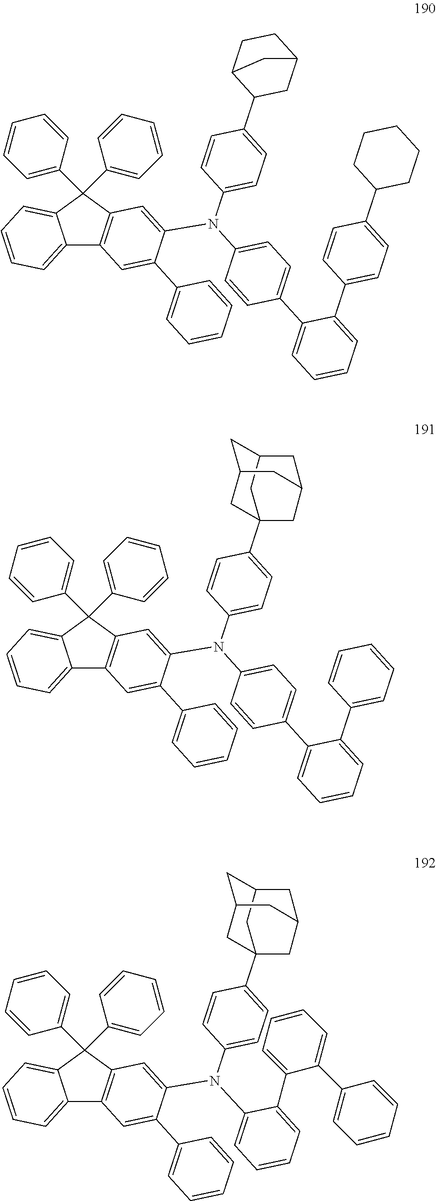 [SMILES: c1ccc(-c2cc3c(cc2N(c2ccc(-c4ccccc4-c4ccc(C5CCCCC5)cc4)cc2)c2ccc(C4CC5CCC4C5)cc2)C(c2ccccc2)(c2ccccc2)c2ccccc2-3)cc1 ; c1ccc(-c2ccccc2-c2ccc(N(c3ccc(C45CC6CC(CC(C6)C4)C5)cc3)c3cc4c(cc3-c3ccccc3)-c3ccccc3C4(c3ccccc3)c3ccccc3)cc2)cc1 ; c1ccc(-c2ccccc2-c2ccccc2N(c2ccc(C34CC5CC(CC(C5)C3)C4)cc2)c2cc3c(cc2-c2ccccc2)-c2ccccc2C3(c2ccccc2)c2ccccc2)cc1] 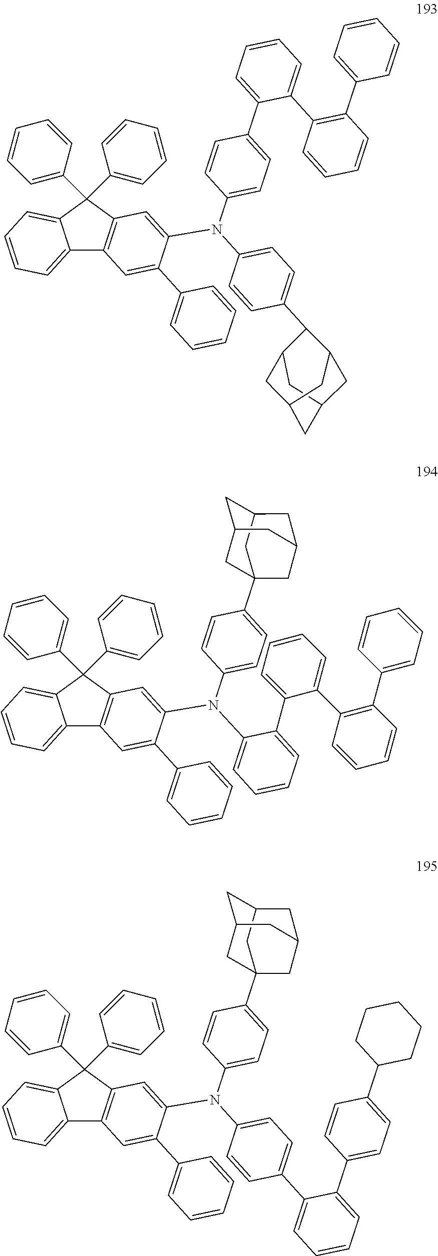 [SMILES: c1ccc(-c2ccccc2-c2ccccc2-c2ccc(N(c3ccc(C4C5CC6CC(C5)CC4C6)cc3)c3cc4c(cc3-c3ccccc3)-c3ccccc3C4(c3ccccc3)c3ccccc3)cc2)cc1 ; c1ccc(-c2ccccc2-c2ccccc2-c2ccccc2N(c2ccc(C34CC5CC(CC(C5)C3)C4)cc2)c2cc3c(cc2-c2ccccc2)-c2ccccc2C3(c2ccccc2)c2ccccc2)cc1 ; c1ccc(-c2cc3c(cc2N(c2ccc(-c4ccccc4-c4ccc(C5CCCCC5)cc4)cc2)c2ccc(C45CC6CC(CC(C6)C4)C5)cc2)C(c2ccccc2)(c2ccccc2)c2ccccc2-3)cc1] 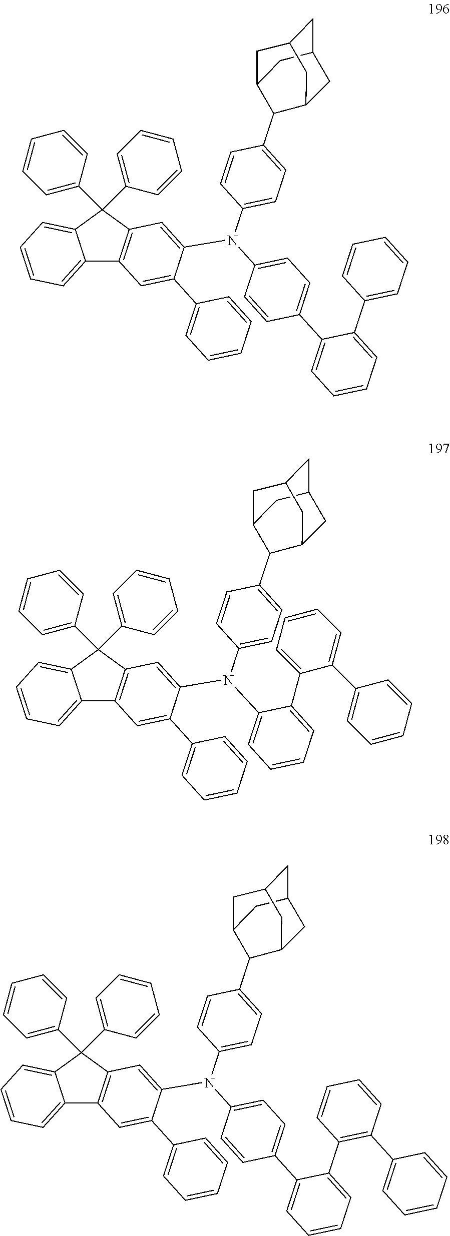 [SMILES: c1ccc(-c2ccccc2-c2ccc(N(c3ccc(C4C5CC6CC(C5)CC4C6)cc3)c3cc4c(cc3-c3ccccc3)-c3ccccc3C4(c3ccccc3)c3ccccc3)cc2)cc1 ; c1ccc(-c2ccccc2-c2ccccc2N(c2ccc(C3C4CC5CC(C4)CC3C5)cc2)c2cc3c(cc2-c2ccccc2)-c2ccccc2C3(c2ccccc2)c2ccccc2)cc1 ; c1ccc(-c2ccccc2-c2ccccc2-c2ccc(N(c3ccc(C4C5CC6CC(C5)CC4C6)cc3)c3cc4c(cc3-c3ccccc3)-c3ccccc3C4(c3ccccc3)c3ccccc3)cc2)cc1] 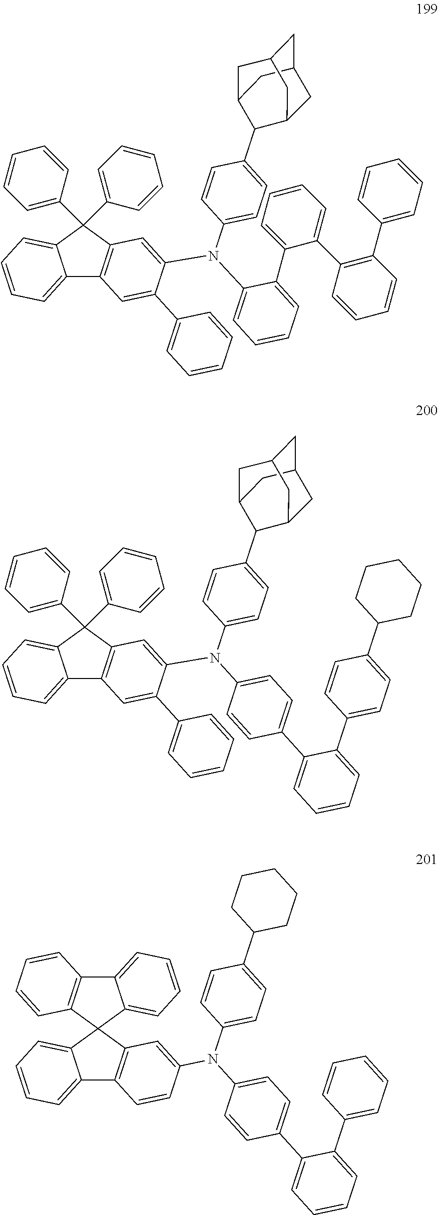 [SMILES: c1ccc(-c2ccccc2-c2ccccc2-c2ccccc2N(c2ccc(C3C4CC5CC(C4)CC3C5)cc2)c2cc3c(cc2-c2ccccc2)-c2ccccc2C3(c2ccccc2)c2ccccc2)cc1 ; c1ccc(-c2cc3c(cc2N(c2ccc(-c4ccccc4-c4ccc(C5CCCCC5)cc4)cc2)c2ccc(C4C5CC6CC(C5)CC4C6)cc2)C(c2ccccc2)(c2ccccc2)c2ccccc2-3)cc1 ; c1ccc(-c2ccccc2-c2ccc(N(c3ccc(C4CCCCC4)cc3)c3ccc4c(c3)C3(c5ccccc5-c5ccccc53)c3ccccc3-4)cc2)cc1] 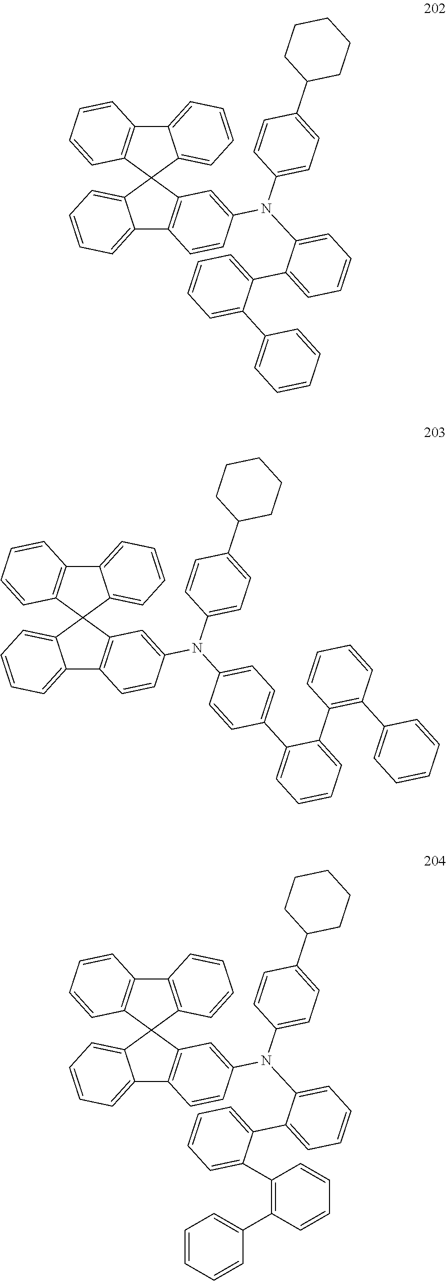 [SMILES: c1ccc(-c2ccccc2-c2ccccc2N(c2ccc(C3CCCCC3)cc2)c2ccc3c(c2)C2(c4ccccc4-c4ccccc42)c2ccccc2-3)cc1 ; c1ccc(-c2ccccc2-c2ccccc2-c2ccc(N(c3ccc(C4CCCCC4)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(C3CCCCC3)cc2)c2ccc3c(c2)C2(c4ccccc4-c4ccccc42)c2ccccc2-3)cc1] 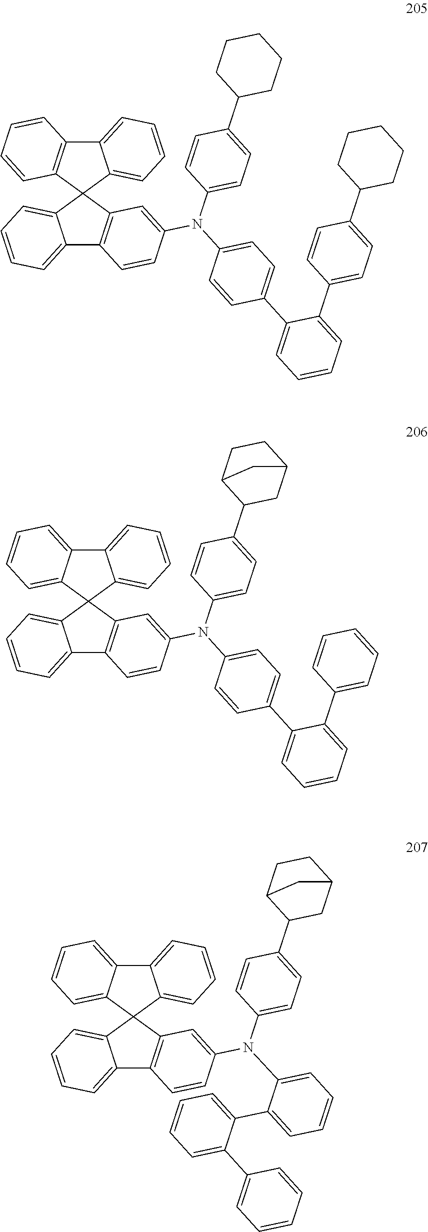 [SMILES: c1ccc(-c2ccc(N(c3ccc(C4CCCCC4)cc3)c3ccc4c(c3)C3(c5ccccc5-c5ccccc53)c3ccccc3-4)cc2)c(-c2ccc(C3CCCCC3)cc2)c1 ; c1ccc(-c2ccccc2-c2ccc(N(c3ccc(C4CC5CCC4C5)cc3)c3ccc4c(c3)C3(c5ccccc5-c5ccccc53)c3ccccc3-4)cc2)cc1 ; c1ccc(-c2ccccc2-c2ccccc2N(c2ccc(C3CC4CCC3C4)cc2)c2ccc3c(c2)C2(c4ccccc4-c4ccccc42)c2ccccc2-3)cc1] 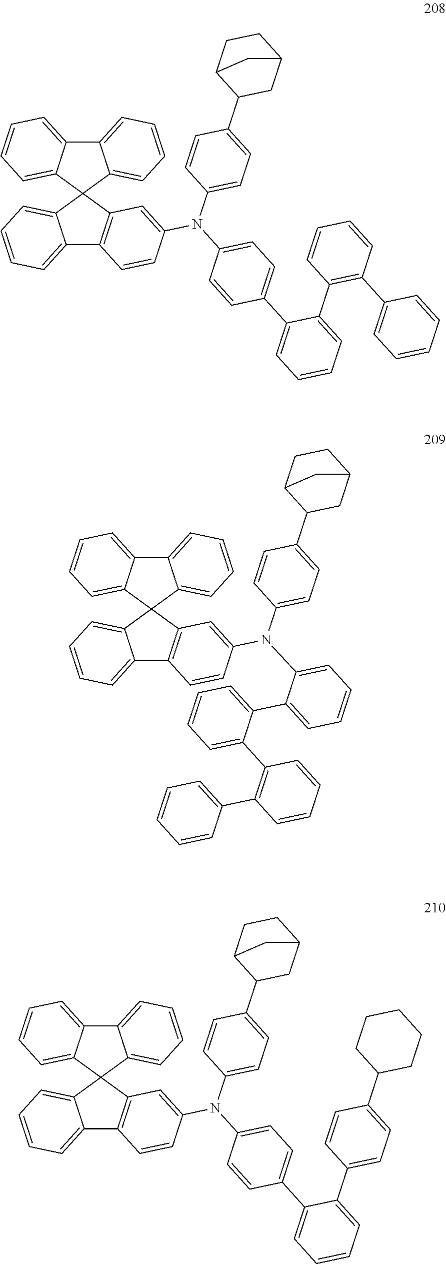 [SMILES: c1ccc(-c2ccccc2-c2ccccc2-c2ccc(N(c3ccc(C4CC5CCC4C5)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(C3CC4CCC3C4)cc2)c2ccc3c(c2)C2(c4ccccc4-c4ccccc42)c2ccccc2-3)cc1 ; c1ccc(-c2ccc(N(c3ccc(C4CC5CCC4C5)cc3)c3ccc4c(c3)C3(c5ccccc5-c5ccccc53)c3ccccc3-4)cc2)c(-c2ccc(C3CCCCC3)cc2)c1] 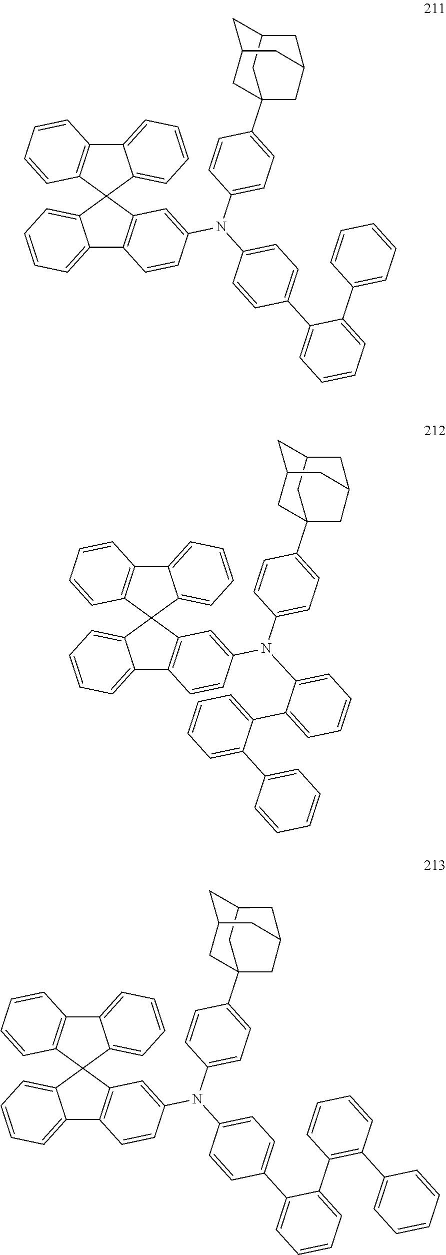 [SMILES: c1ccc(-c2ccccc2-c2ccc(N(c3ccc(C45CC6CC(CC(C6)C4)C5)cc3)c3ccc4c(c3)C3(c5ccccc5-c5ccccc53)c3ccccc3-4)cc2)cc1 ; c1ccc(-c2ccccc2-c2ccccc2N(c2ccc(C34CC5CC(CC(C5)C3)C4)cc2)c2ccc3c(c2)C2(c4ccccc4-c4ccccc42)c2ccccc2-3)cc1 ; c1ccc(-c2ccccc2-c2ccccc2-c2ccc(N(c3ccc(C45CC6CC(CC(C6)C4)C5)cc3)c3ccc4c(c3)C3(c5ccccc5-c5ccccc53)c3ccccc3-4)cc2)cc1] 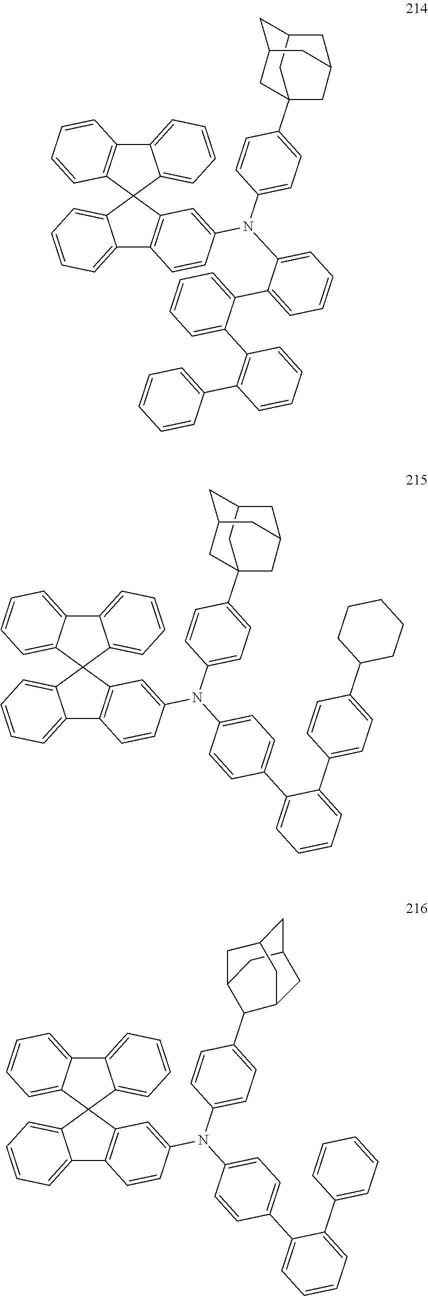 [SMILES: c1ccc(-c2ccccc2-c2ccccc2-c2ccccc2N(c2ccc(C34CC5CC(CC(C5)C3)C4)cc2)c2ccc3c(c2)C2(c4ccccc4-c4ccccc42)c2ccccc2-3)cc1 ; c1ccc(-c2ccc(N(c3ccc(C45CC6CC(CC(C6)C4)C5)cc3)c3ccc4c(c3)C3(c5ccccc5-c5ccccc53)c3ccccc3-4)cc2)c(-c2ccc(C3CCCCC3)cc2)c1 ; c1ccc(-c2ccccc2-c2ccc(N(c3ccc(C4C5CC6CC(C5)CC4C6)cc3)c3ccc4c(c3)C3(c5ccccc5-c5ccccc53)c3ccccc3-4)cc2)cc1] 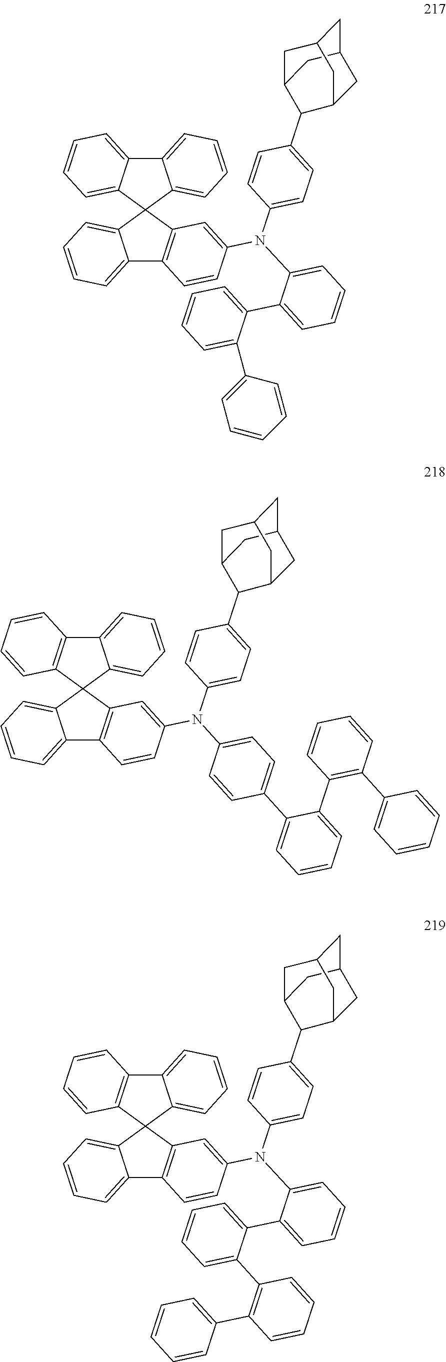 [SMILES: c1ccc(-c2ccccc2-c2ccccc2N(c2ccc(C3C4CC5CC(C4)CC3C5)cc2)c2ccc3c(c2)C2(c4ccccc4-c4ccccc42)c2ccccc2-3)cc1 ; c1ccc(-c2ccccc2-c2ccccc2-c2ccc(N(c3ccc(C4C5CC6CC(C5)CC4C6)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(C3C4CC5CC(C4)CC3C5)cc2)c2ccc3c(c2)C2(c4ccccc4-c4ccccc42)c2ccccc2-3)cc1] 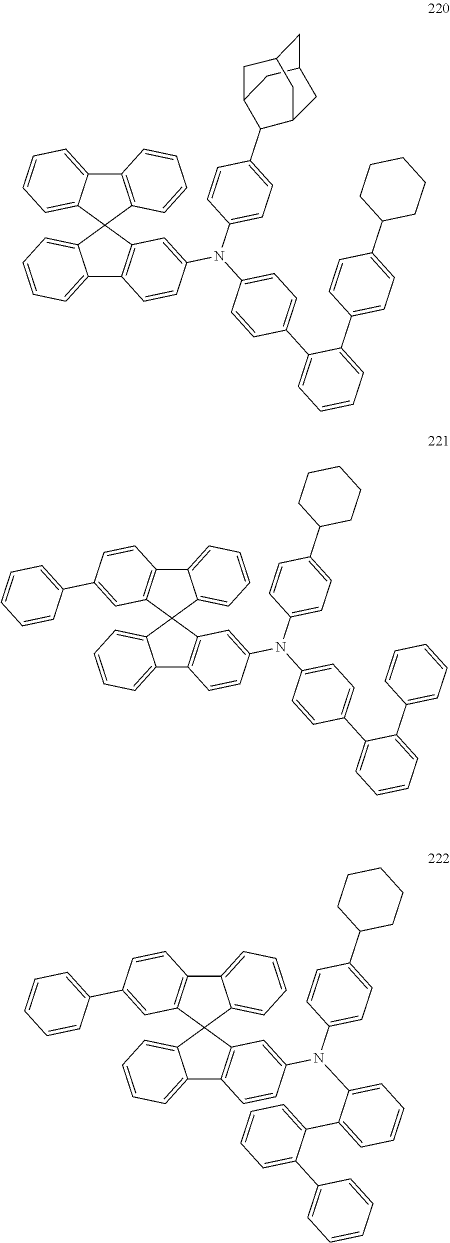 [SMILES: c1ccc(-c2ccc(N(c3ccc(C4C5CC6CC(C5)CC4C6)cc3)c3ccc4c(c3)C3(c5ccccc5-c5ccccc53)c3ccccc3-4)cc2)c(-c2ccc(C3CCCCC3)cc2)c1 ; c1ccc(-c2ccc3c(c2)C2(c4ccccc4-3)c3ccccc3-c3ccc(N(c4ccc(-c5ccccc5-c5ccccc5)cc4)c4ccc(C5CCCCC5)cc4)cc32)cc1 ; c1ccc(-c2ccc3c(c2)C2(c4ccccc4-3)c3ccccc3-c3ccc(N(c4ccc(C5CCCCC5)cc4)c4ccccc4-c4ccccc4-c4ccccc4)cc32)cc1] 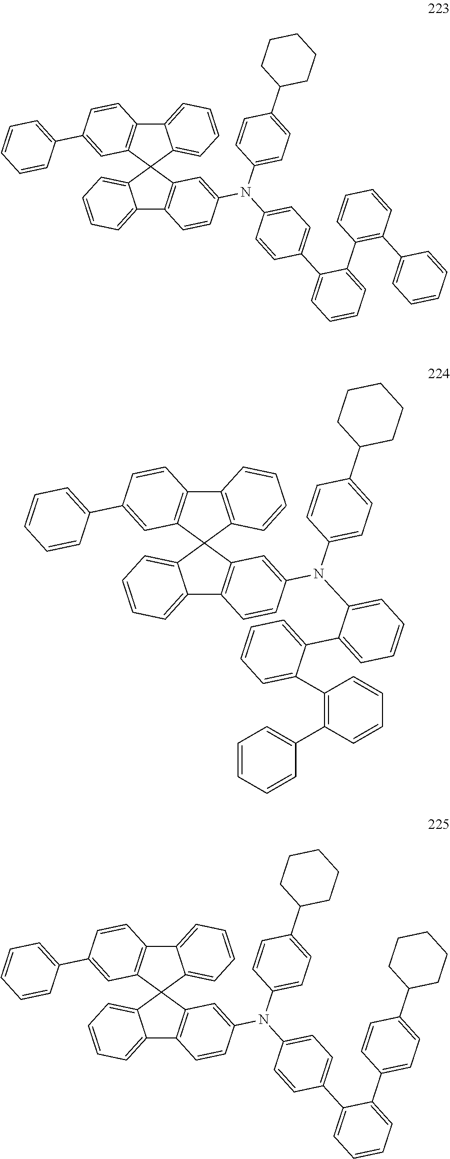 [SMILES: c1ccc(-c2ccc3c(c2)C2(c4ccccc4-3)c3ccccc3-c3ccc(N(c4ccc(-c5ccccc5-c5ccccc5-c5ccccc5)cc4)c4ccc(C5CCCCC5)cc4)cc32)cc1 ; c1ccc(-c2ccc3c(c2)C2(c4ccccc4-3)c3ccccc3-c3ccc(N(c4ccc(C5CCCCC5)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccc(C6CCCCC6)cc5)cc4)c4ccc(C5CCCCC5)cc4)cc32)cc1] 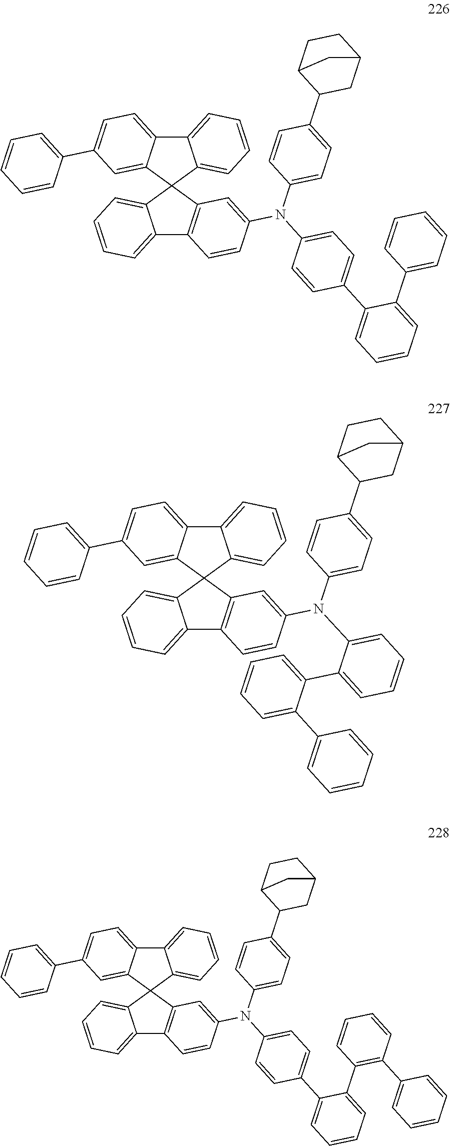 [SMILES: c1ccc(-c2ccc3c(c2)C2(c4ccccc4-3)c3ccccc3-c3ccc(N(c4ccc(-c5ccccc5-c5ccccc5)cc4)c4ccc(C5CC6CCC5C6)cc4)cc32)cc1 ; c1ccc(-c2ccc3c(c2)C2(c4ccccc4-3)c3ccccc3-c3ccc(N(c4ccc(C5CC6CCC5C6)cc4)c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccccc5-c5ccccc5)cc4)c4ccc(C5CC6CCC5C6)cc4)cc32)cc1] 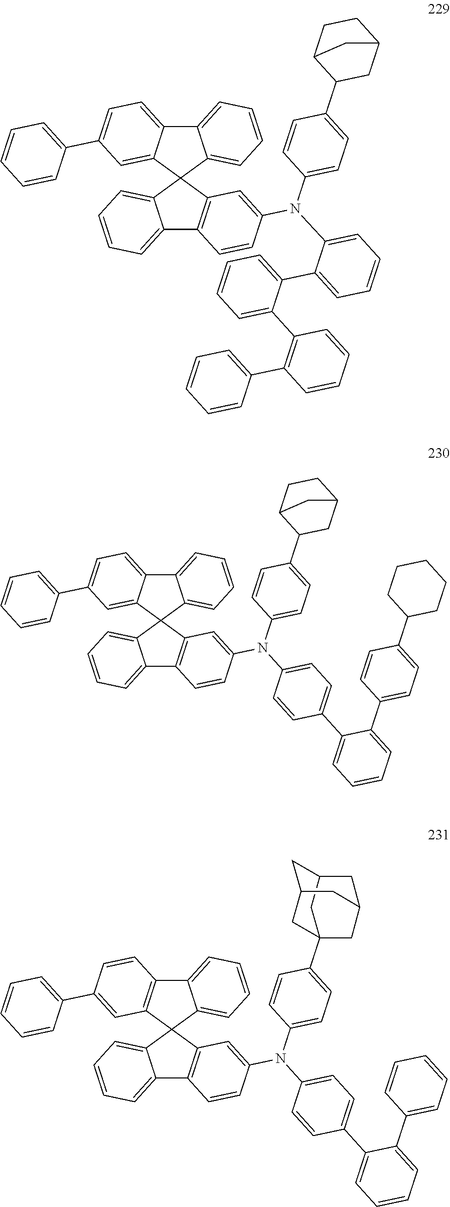 [SMILES: c1ccc(-c2ccc3c(c2)C2(c4ccccc4-3)c3ccccc3-c3ccc(N(c4ccc(C5CC6CCC5C6)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccc(C6CCCCC6)cc5)cc4)c4ccc(C5CC6CCC5C6)cc4)cc32)cc1 ; c1ccc(-c2ccc3c(c2)C2(c4ccccc4-3)c3ccccc3-c3ccc(N(c4ccc(-c5ccccc5-c5ccccc5)cc4)c4ccc(C56CC7CC(CC(C7)C5)C6)cc4)cc32)cc1] 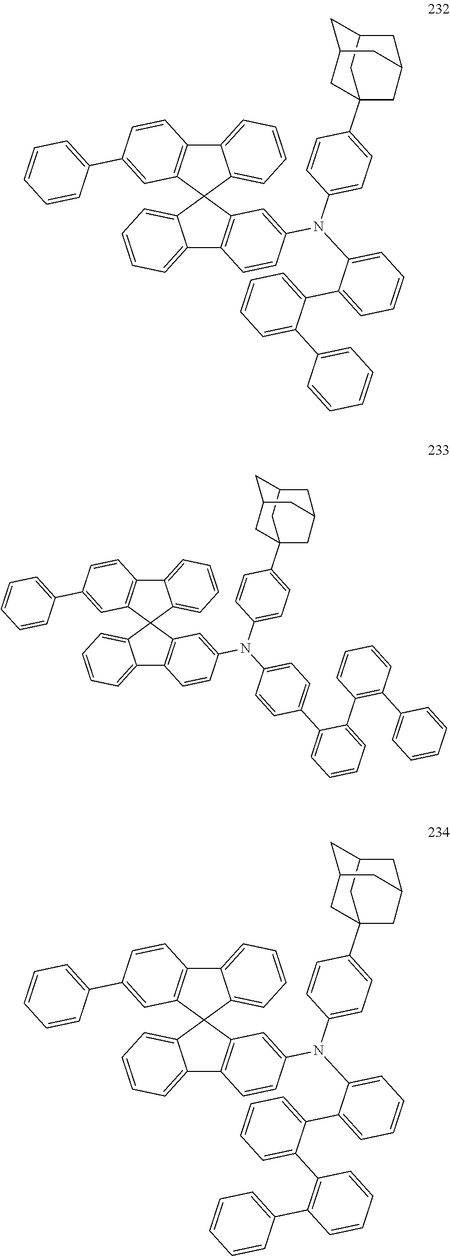 [SMILES: c1ccc(-c2ccc3c(c2)C2(c4ccccc4-3)c3ccccc3-c3ccc(N(c4ccc(C56CC7CC(CC(C7)C5)C6)cc4)c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccccc5-c5ccccc5)cc4)c4ccc(C56CC7CC(CC(C7)C5)C6)cc4)cc32)cc1 ; c1ccc(-c2ccc3c(c2)C2(c4ccccc4-3)c3ccccc3-c3ccc(N(c4ccc(C56CC7CC(CC(C7)C5)C6)cc4)c4ccccc4-c4ccccc4-c4ccccc4-c4ccccc4)cc32)cc1] 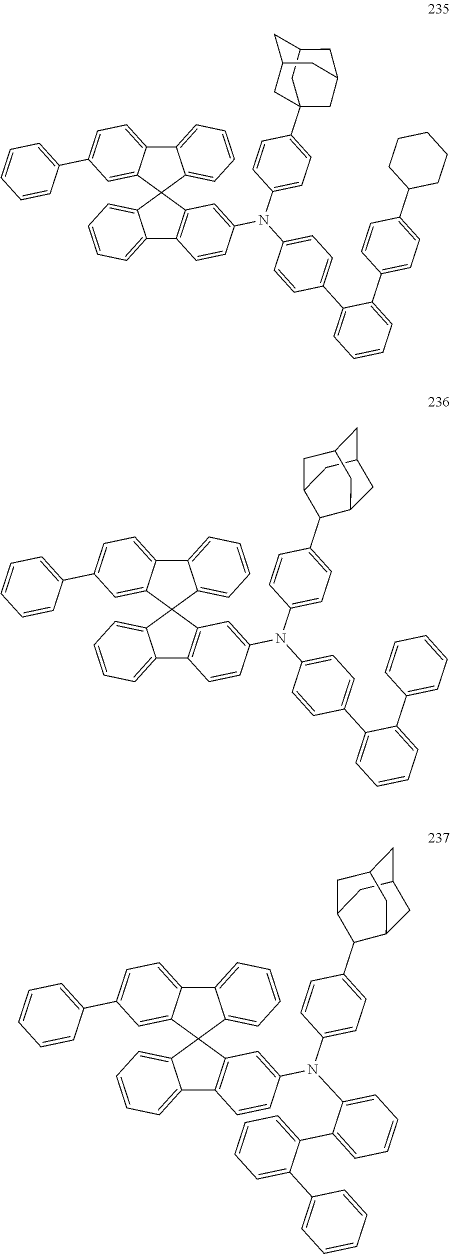 [SMILES: c1ccc(-c2ccc3c(c2)C2(c4ccccc4-3)c3ccccc3-c3ccc(N(c4ccc(-c5ccccc5-c5ccc(C6CCCCC6)cc5)cc4)c4ccc(C56CC7CC(CC(C7)C5)C6)cc4)cc32)cc1 ; c1ccc(-c2ccc3c(c2)C2(c4ccccc4-3)c3ccccc3-c3ccc(N(c4ccc(-c5ccccc5-c5ccccc5)cc4)c4ccc(C5C6CC7CC(C6)CC5C7)cc4)cc32)cc1 ; c1ccc(-c2ccc3c(c2)C2(c4ccccc4-3)c3ccccc3-c3ccc(N(c4ccc(C5C6CC7CC(C6)CC5C7)cc4)c4ccccc4-c4ccccc4-c4ccccc4)cc32)cc1] 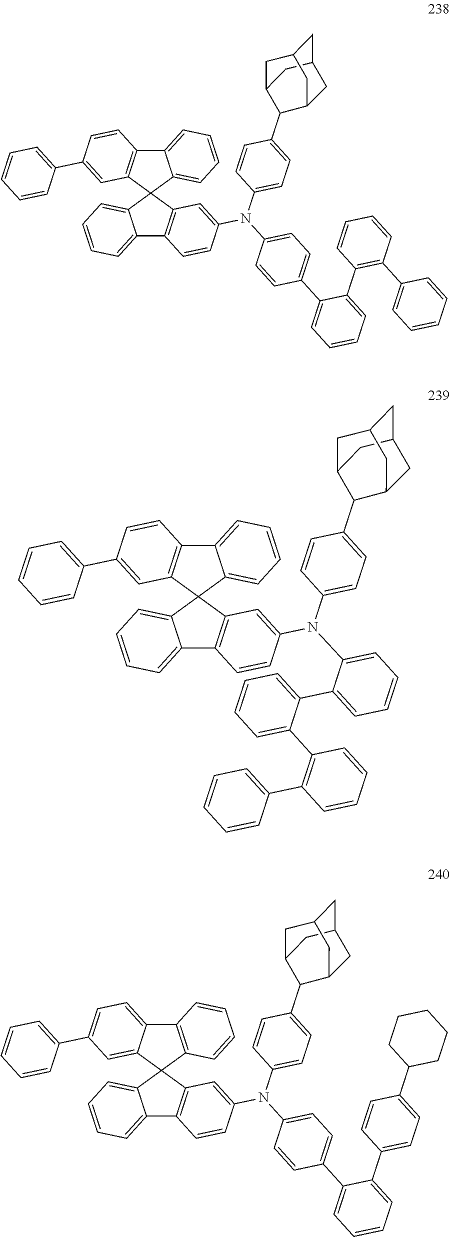 [SMILES: c1ccc(-c2ccc3c(c2)C2(c4ccccc4-3)c3ccccc3-c3ccc(N(c4ccc(-c5ccccc5-c5ccccc5-c5ccccc5)cc4)c4ccc(C5C6CC7CC(C6)CC5C7)cc4)cc32)cc1 ; c1ccc(-c2ccc3c(c2)C2(c4ccccc4-3)c3ccccc3-c3ccc(N(c4ccc(C5C6CC7CC(C6)CC5C7)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccccc5-c5ccc(C6CCCCC6)cc5)cc4)c4ccc(C5C6CC7CC(C6)CC5C7)cc4)cc32)cc1] 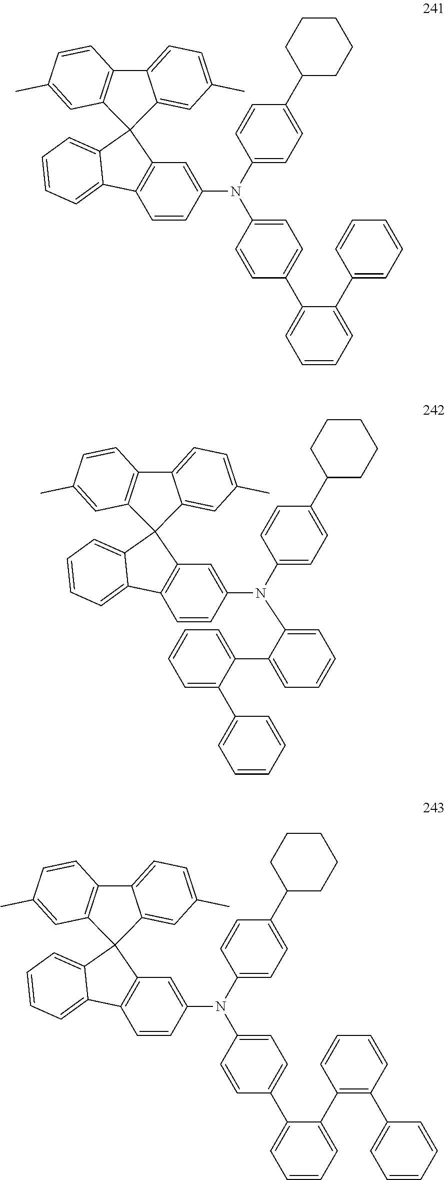 [SMILES: Cc1ccc2c(c1)C1(c3ccccc3-c3ccc(N(c4ccc(-c5ccccc5-c5ccccc5)cc4)c4ccc(C5CCCCC5)cc4)cc31)c1cc(C)ccc1-2 ; Cc1ccc2c(c1)C1(c3ccccc3-c3ccc(N(c4ccc(C5CCCCC5)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccccc5-c5ccccc5)cc4)c4ccc(C5CCCCC5)cc4)cc31)c1cc(C)ccc1-2] 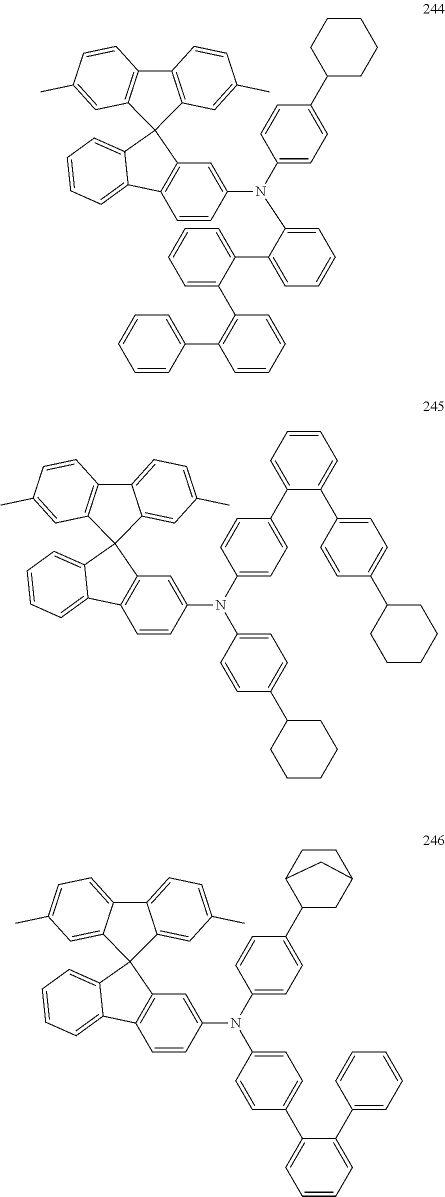 [SMILES: Cc1ccc2c(c1)C1(c3ccccc3-c3ccc(N(c4ccc(C5CCCCC5)cc4)c4ccccc4-c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccc(C6CCCCC6)cc5)cc4)c4ccc(C5CCCCC5)cc4)cc31)c1cc(C)ccc1-2 ; Cc1ccc2c(c1)C1(c3ccccc3-c3ccc(N(c4ccc(-c5ccccc5-c5ccccc5)cc4)c4ccc(C5CC6CCC5C6)cc4)cc31)c1cc(C)ccc1-2] 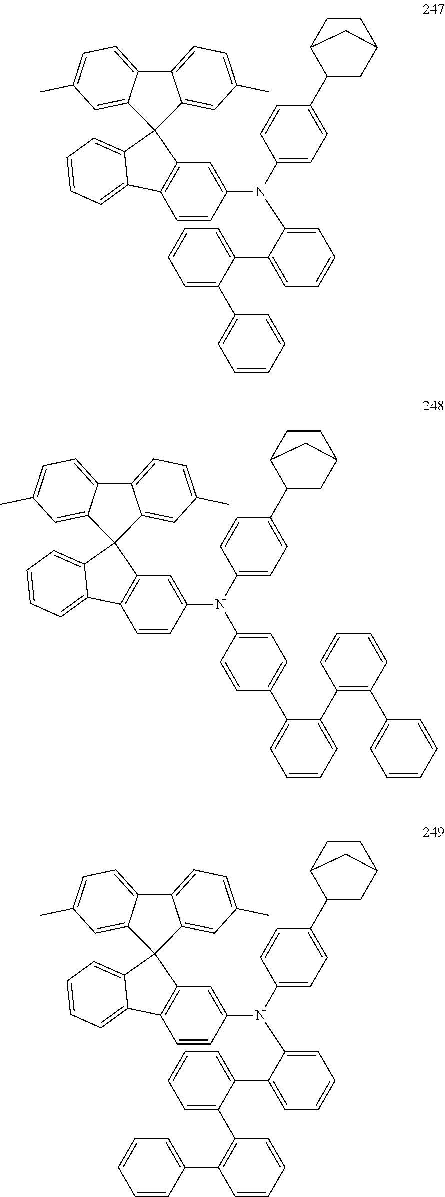 [SMILES: Cc1ccc2c(c1)C1(c3ccccc3-c3ccc(N(c4ccc(C5CC6CCC5C6)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccccc5-c5ccccc5)cc4)c4ccc(C5CC6CCC5C6)cc4)cc31)c1cc(C)ccc1-2 ; Cc1ccc2c(c1)C1(c3ccccc3-c3ccc(N(c4ccc(C5CC6CCC5C6)cc4)c4ccccc4-c4ccccc4-c4ccccc4-c4ccccc4)cc31)c1cc(C)ccc1-2] 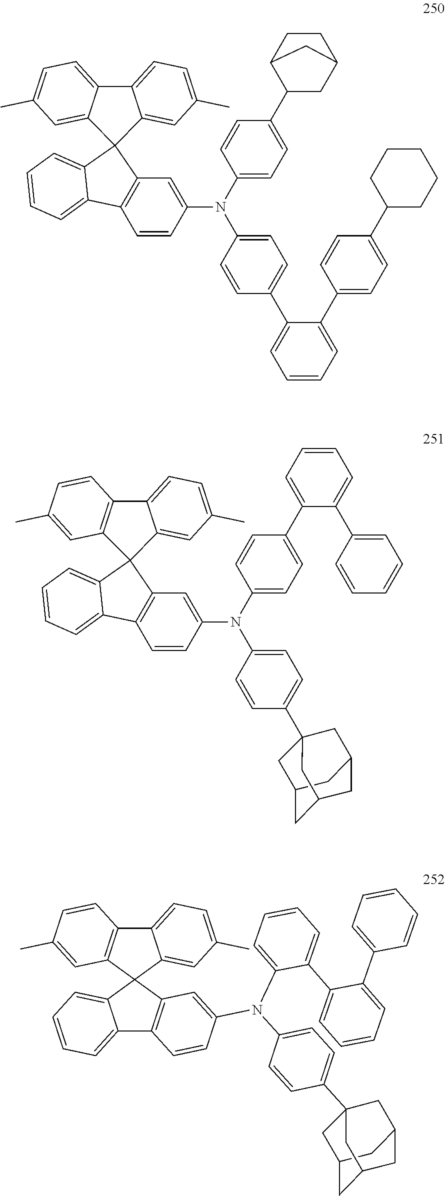 [SMILES: Cc1ccc2c(c1)C1(c3ccccc3-c3ccc(N(c4ccc(-c5ccccc5-c5ccc(C6CCCCC6)cc5)cc4)c4ccc(C5CC6CCC5C6)cc4)cc31)c1cc(C)ccc1-2 ; Cc1ccc2c(c1)C1(c3ccccc3-c3ccc(N(c4ccc(-c5ccccc5-c5ccccc5)cc4)c4ccc(C56CC7CC(CC(C7)C5)C6)cc4)cc31)c1cc(C)ccc1-2 ; Cc1ccc2c(c1)C1(c3ccccc3-c3ccc(N(c4ccc(C56CC7CC(CC(C7)C5)C6)cc4)c4ccccc4-c4ccccc4-c4ccccc4)cc31)c1cc(C)ccc1-2] 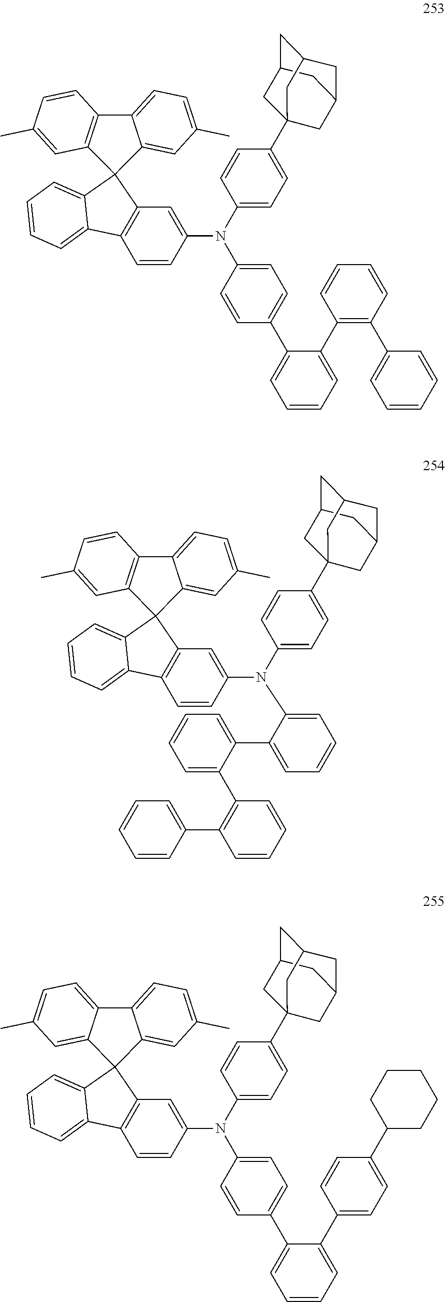 [SMILES: Cc1ccc2c(c1)C1(c3ccccc3-c3ccc(N(c4ccc(-c5ccccc5-c5ccccc5-c5ccccc5)cc4)c4ccc(C56CC7CC(CC(C7)C5)C6)cc4)cc31)c1cc(C)ccc1-2 ; Cc1ccc2c(c1)C1(c3ccccc3-c3ccc(N(c4ccc(C56CC7CC(CC(C7)C5)C6)cc4)c4ccccc4-c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccc(C6CCCCC6)cc5)cc4)c4ccc(C56CC7CC(CC(C7)C5)C6)cc4)cc31)c1cc(C)ccc1-2] 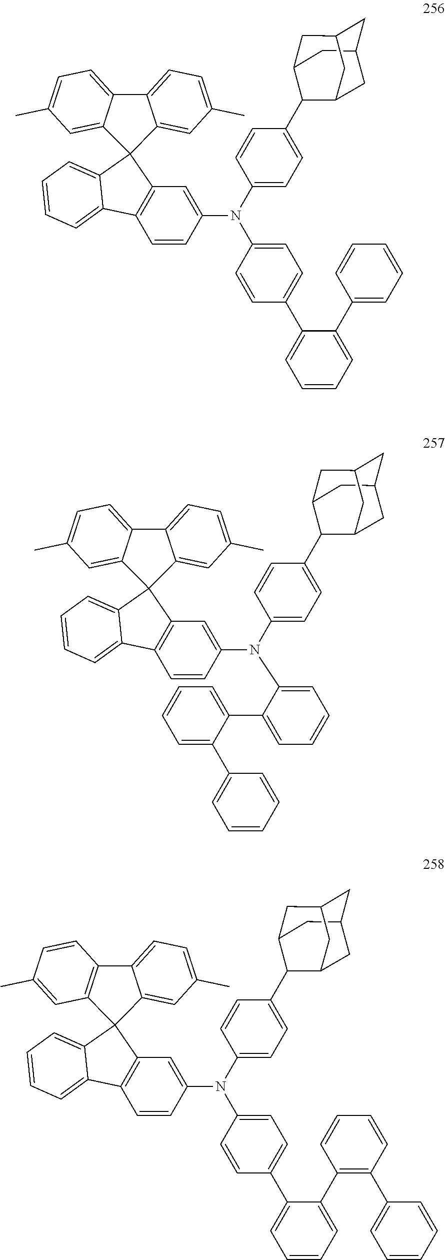 [SMILES: Cc1ccc2c(c1)C1(c3ccccc3-c3ccc(N(c4ccc(-c5ccccc5-c5ccccc5)cc4)c4ccc(C5C6CC7CC(C6)CC5C7)cc4)cc31)c1cc(C)ccc1-2 ; Cc1ccc2c(c1)C1(c3ccccc3-c3ccc(N(c4ccc(C5C6CC7CC(C6)CC5C7)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccccc5-c5ccccc5)cc4)c4ccc(C5C6CC7CC(C6)CC5C7)cc4)cc31)c1cc(C)ccc1-2] 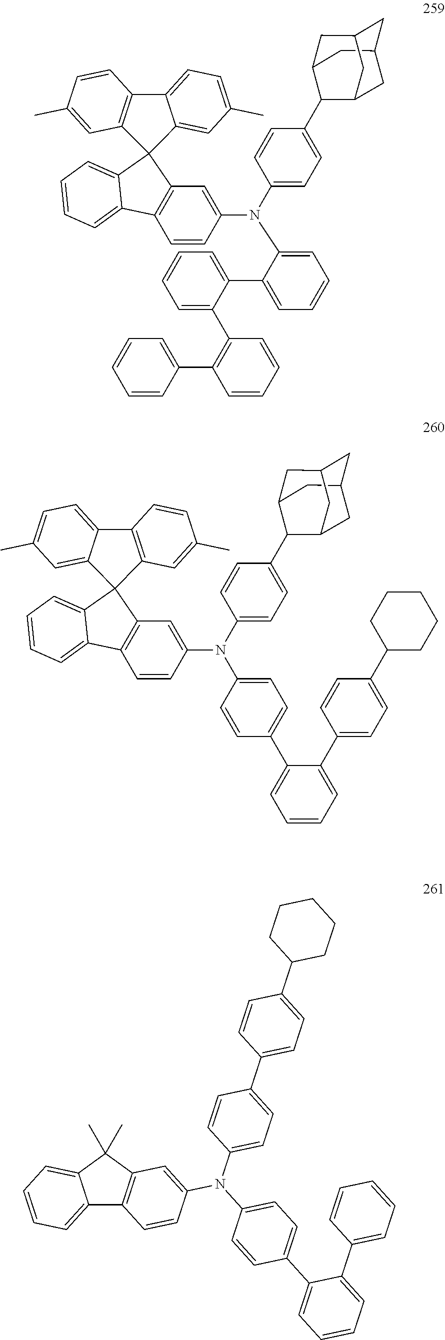 [SMILES: Cc1ccc2c(c1)C1(c3ccccc3-c3ccc(N(c4ccc(C5C6CC7CC(C6)CC5C7)cc4)c4ccccc4-c4ccccc4-c4ccccc4-c4ccccc4)cc31)c1cc(C)ccc1-2 ; Cc1ccc2c(c1)C1(c3ccccc3-c3ccc(N(c4ccc(-c5ccccc5-c5ccc(C6CCCCC6)cc5)cc4)c4ccc(C5C6CC7CC(C6)CC5C7)cc4)cc31)c1cc(C)ccc1-2 ; CC1(C)c2ccccc2-c2ccc(N(c3ccc(-c4ccc(C5CCCCC5)cc4)cc3)c3ccc(-c4ccccc4-c4ccccc4)cc3)cc21] 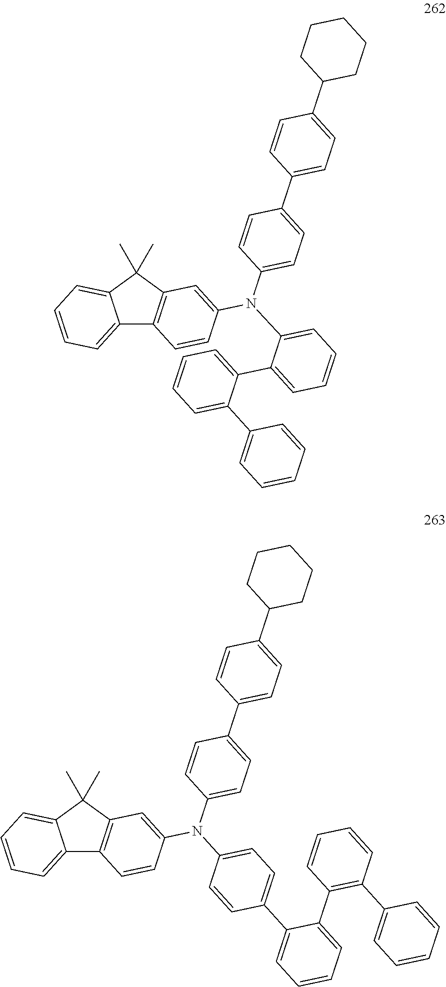 [SMILES: CC1(C)c2ccccc2-c2ccc(N(c3ccc(-c4ccc(C5CCCCC5)cc4)cc3)c3ccccc3-c3ccccc3-c3ccccc3)cc21 ; CC1(C)c2ccccc2-c2ccc(N(c3ccc(-c4ccc(C5CCCCC5)cc4)cc3)c3ccc(-c4ccccc4-c4ccccc4-c4ccccc4)cc3)cc21] 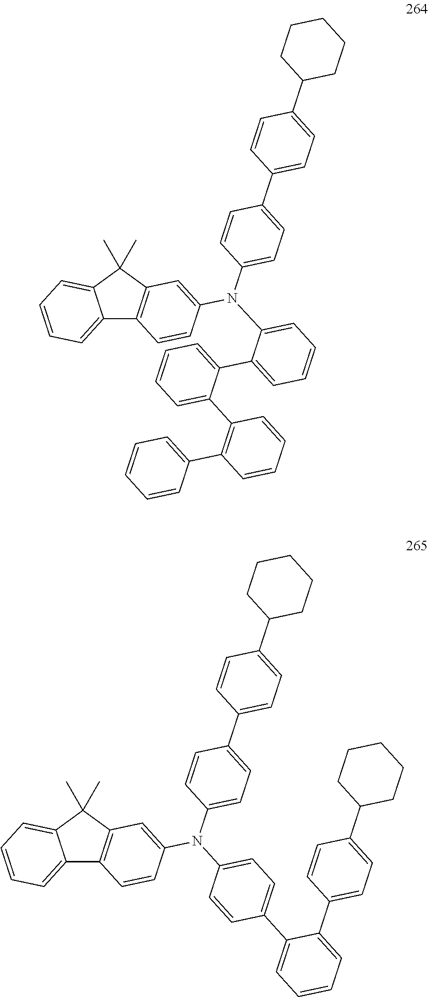 [SMILES: CC1(C)c2ccccc2-c2ccc(N(c3ccc(-c4ccc(C5CCCCC5)cc4)cc3)c3ccccc3-c3ccccc3-c3ccccc3-c3ccccc3)cc21 ; CC1(C)c2ccccc2-c2ccc(N(c3ccc(-c4ccc(C5CCCCC5)cc4)cc3)c3ccc(-c4ccccc4-c4ccc(C5CCCCC5)cc4)cc3)cc21] 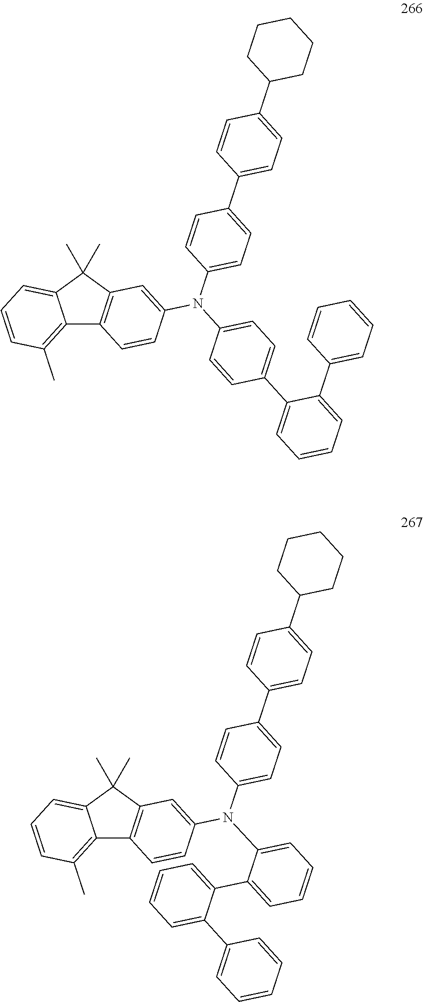 [SMILES: Cc1cccc2c1-c1ccc(N(c3ccc(-c4ccc(C5CCCCC5)cc4)cc3)c3ccc(-c4ccccc4-c4ccccc4)cc3)cc1C2(C)C ; Cc1cccc2c1-c1ccc(N(c3ccc(-c4ccc(C5CCCCC5)cc4)cc3)c3ccccc3-c3ccccc3-c3ccccc3)cc1C2(C)C] 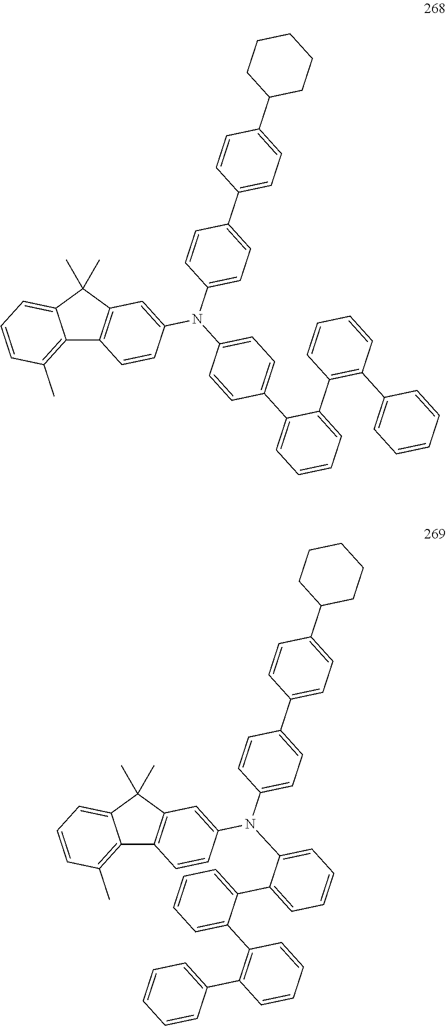 [SMILES: Cc1cccc2c1-c1ccc(N(c3ccc(-c4ccc(C5CCCCC5)cc4)cc3)c3ccc(-c4ccccc4-c4ccccc4-c4ccccc4)cc3)cc1C2(C)C ; Cc1cccc2c1-c1ccc(N(c3ccc(-c4ccc(C5CCCCC5)cc4)cc3)c3ccccc3-c3ccccc3-c3ccccc3-c3ccccc3)cc1C2(C)C] 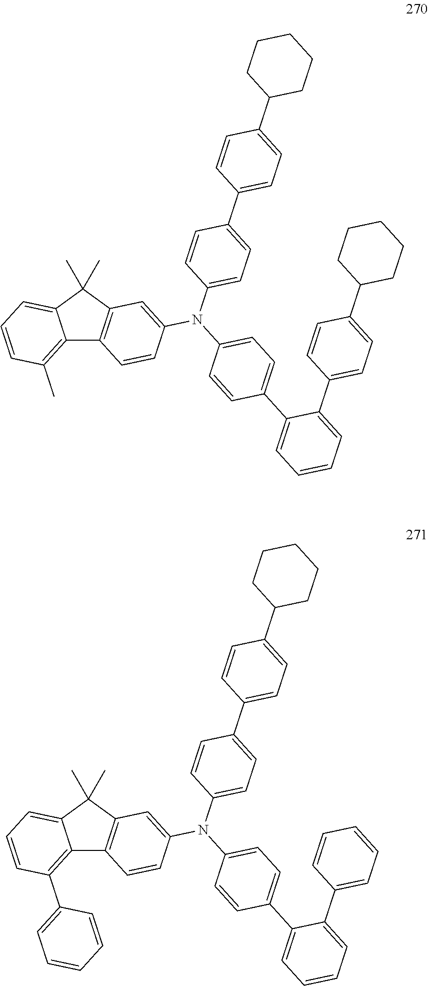 [SMILES: Cc1cccc2c1-c1ccc(N(c3ccc(-c4ccc(C5CCCCC5)cc4)cc3)c3ccc(-c4ccccc4-c4ccc(C5CCCCC5)cc4)cc3)cc1C2(C)C ; CC1(C)c2cc(N(c3ccc(-c4ccc(C5CCCCC5)cc4)cc3)c3ccc(-c4ccccc4-c4ccccc4)cc3)ccc2-c2c(-c3ccccc3)cccc21] 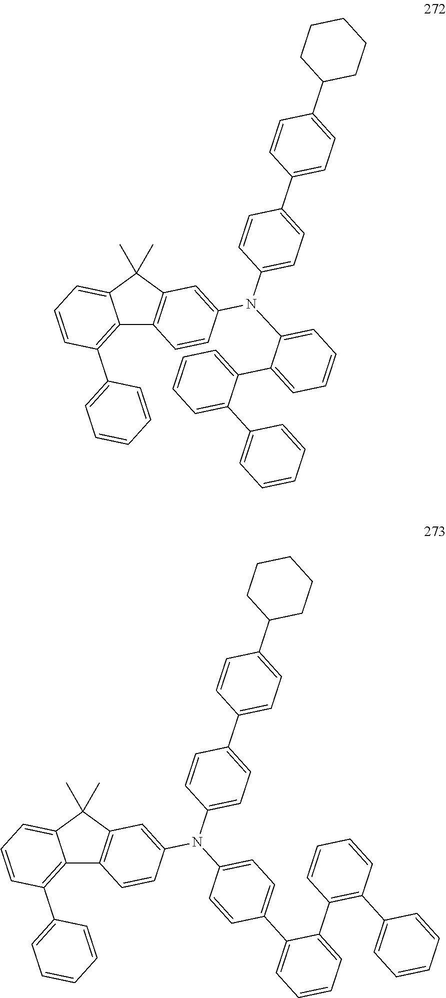 [SMILES: CC1(C)c2cc(N(c3ccc(-c4ccc(C5CCCCC5)cc4)cc3)c3ccccc3-c3ccccc3-c3ccccc3)ccc2-c2c(-c3ccccc3)cccc21 ; CC1(C)c2cc(N(c3ccc(-c4ccc(C5CCCCC5)cc4)cc3)c3ccc(-c4ccccc4-c4ccccc4-c4ccccc4)cc3)ccc2-c2c(-c3ccccc3)cccc21] 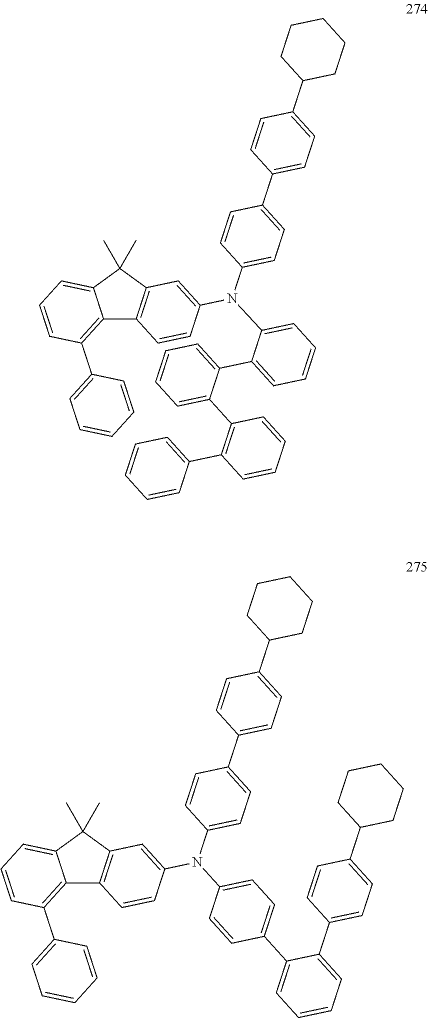 [SMILES: CC1(C)c2cc(N(c3ccc(-c4ccc(C5CCCCC5)cc4)cc3)c3ccccc3-c3ccccc3-c3ccccc3-c3ccccc3)ccc2-c2c(-c3ccccc3)cccc21 ; CC1(C)c2cc(N(c3ccc(-c4ccc(C5CCCCC5)cc4)cc3)c3ccc(-c4ccccc4-c4ccc(C5CCCCC5)cc4)cc3)ccc2-c2c(-c3ccccc3)cccc21] 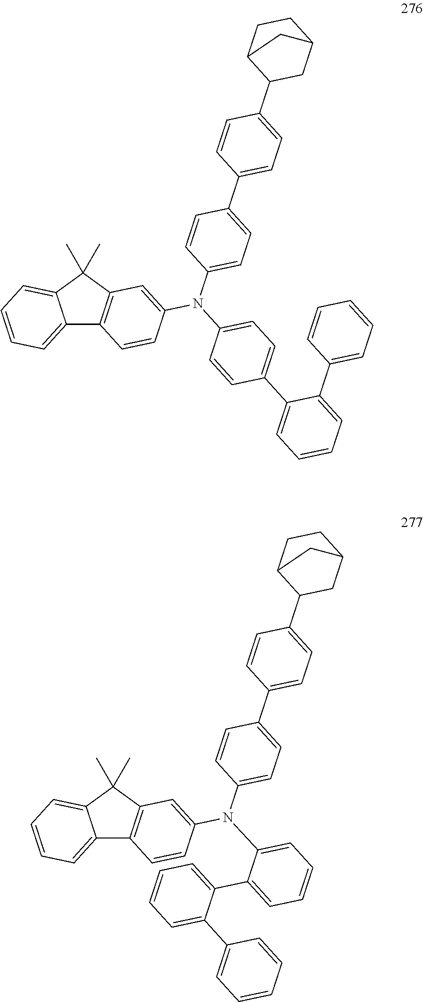 [SMILES: CC1(C)c2ccccc2-c2ccc(N(c3ccc(-c4ccc(C5CC6CCC5C6)cc4)cc3)c3ccc(-c4ccccc4-c4ccccc4)cc3)cc21 ; CC1(C)c2ccccc2-c2ccc(N(c3ccc(-c4ccc(C5CC6CCC5C6)cc4)cc3)c3ccccc3-c3ccccc3-c3ccccc3)cc21] 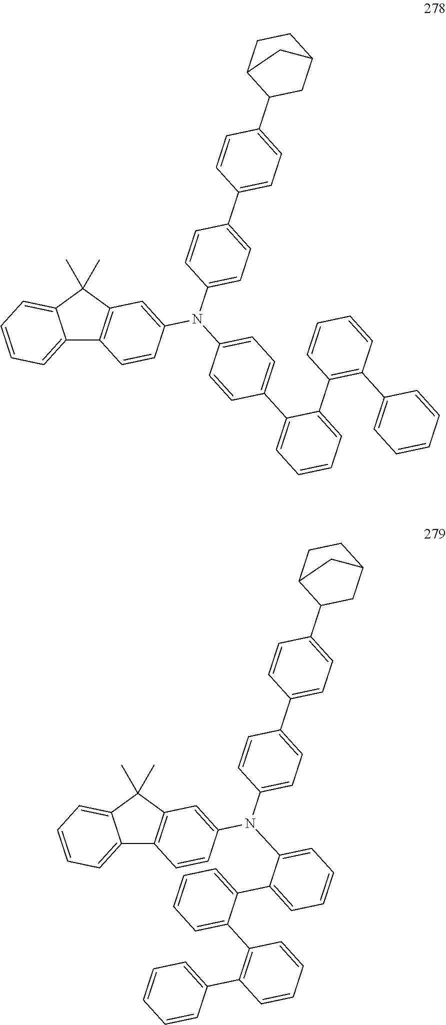 [SMILES: CC1(C)c2ccccc2-c2ccc(N(c3ccc(-c4ccc(C5CC6CCC5C6)cc4)cc3)c3ccc(-c4ccccc4-c4ccccc4-c4ccccc4)cc3)cc21 ; CC1(C)c2ccccc2-c2ccc(N(c3ccc(-c4ccc(C5CC6CCC5C6)cc4)cc3)c3ccccc3-c3ccccc3-c3ccccc3-c3ccccc3)cc21] 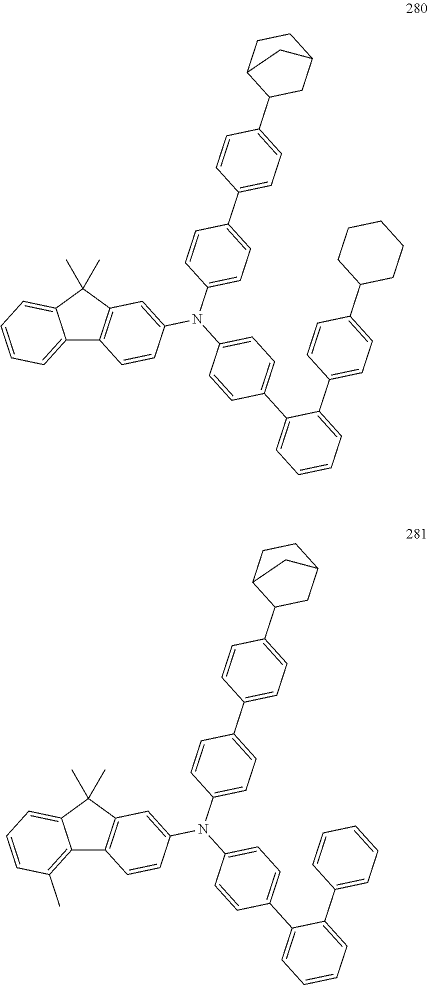 [SMILES: CC1(C)c2ccccc2-c2ccc(N(c3ccc(-c4ccc(C5CC6CCC5C6)cc4)cc3)c3ccc(-c4ccccc4-c4ccc(C5CCCCC5)cc4)cc3)cc21 ; Cc1cccc2c1-c1ccc(N(c3ccc(-c4ccc(C5CC6CCC5C6)cc4)cc3)c3ccc(-c4ccccc4-c4ccccc4)cc3)cc1C2(C)C] 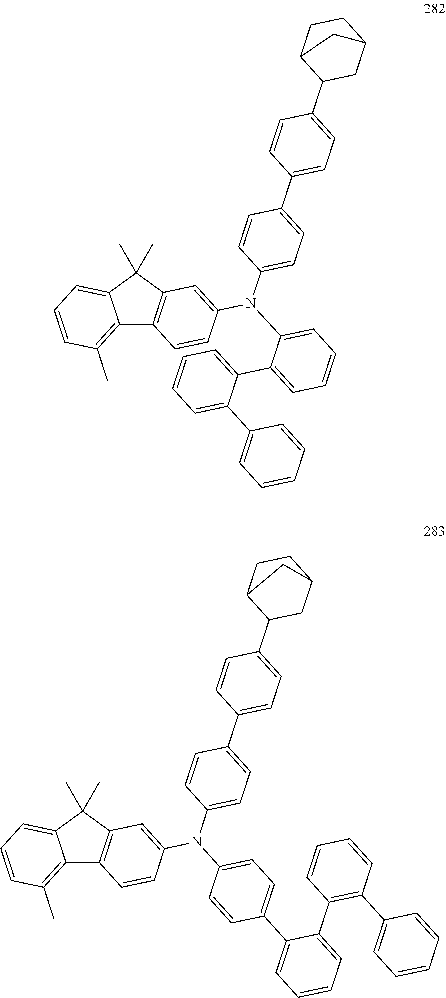 [SMILES: Cc1cccc2c1-c1ccc(N(c3ccc(-c4ccc(C5CC6CCC5C6)cc4)cc3)c3ccccc3-c3ccccc3-c3ccccc3)cc1C2(C)C ; Cc1cccc2c1-c1ccc(N(c3ccc(-c4ccc(C5CC6CCC5C6)cc4)cc3)c3ccc(-c4ccccc4-c4ccccc4-c4ccccc4)cc3)cc1C2(C)C] 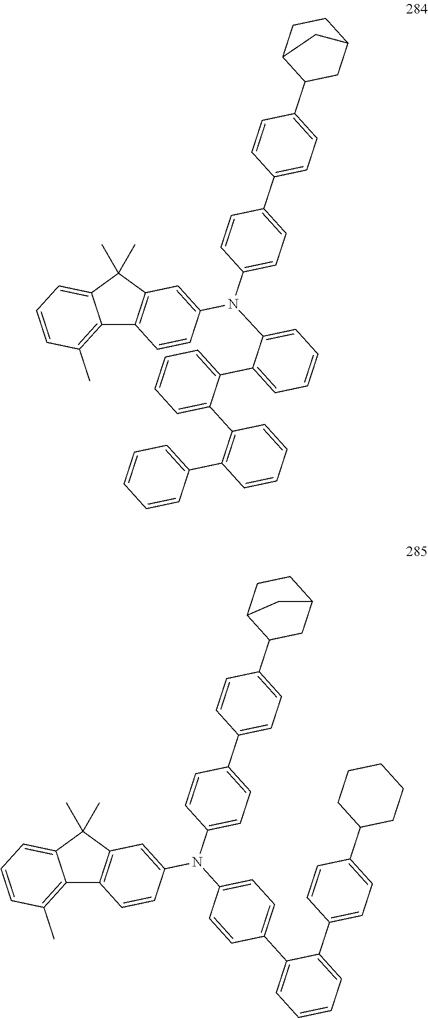 [SMILES: Cc1cccc2c1-c1ccc(N(c3ccc(-c4ccc(C5CC6CCC5C6)cc4)cc3)c3ccccc3-c3ccccc3-c3ccccc3-c3ccccc3)cc1C2(C)C ; Cc1cccc2c1-c1ccc(N(c3ccc(-c4ccc(C5CC6CCC5C6)cc4)cc3)c3ccc(-c4ccccc4-c4ccc(C5CCCCC5)cc4)cc3)cc1C2(C)C] 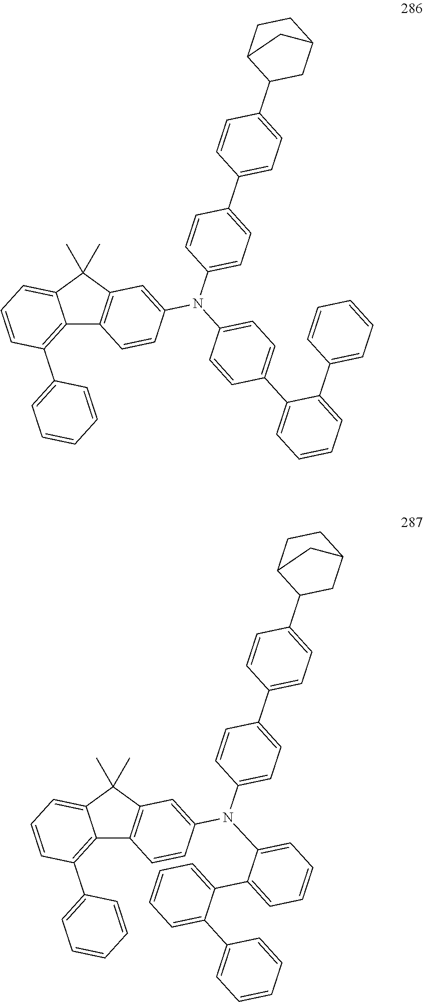 [SMILES: CC1(C)c2cc(N(c3ccc(-c4ccc(C5CC6CCC5C6)cc4)cc3)c3ccc(-c4ccccc4-c4ccccc4)cc3)ccc2-c2c(-c3ccccc3)cccc21 ; CC1(C)c2cc(N(c3ccc(-c4ccc(C5CC6CCC5C6)cc4)cc3)c3ccccc3-c3ccccc3-c3ccccc3)ccc2-c2c(-c3ccccc3)cccc21] 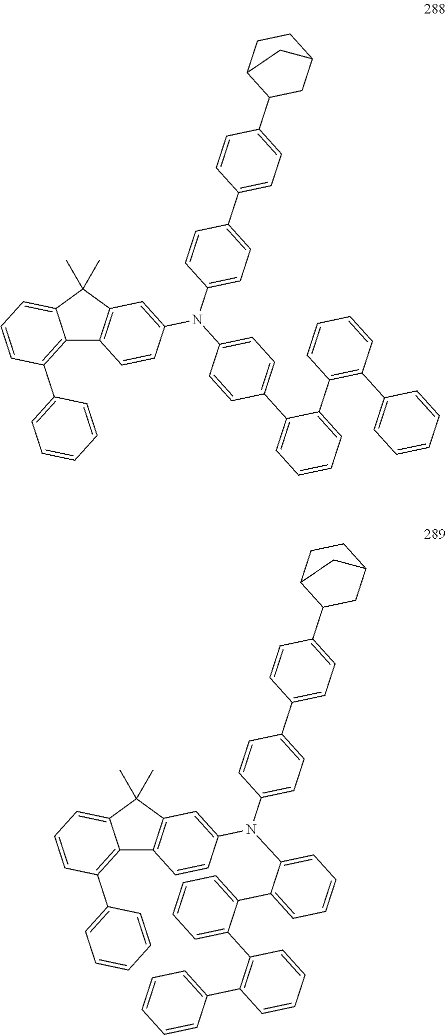 [SMILES: CC1(C)c2cc(N(c3ccc(-c4ccc(C5CC6CCC5C6)cc4)cc3)c3ccc(-c4ccccc4-c4ccccc4-c4ccccc4)cc3)ccc2-c2c(-c3ccccc3)cccc21 ; CC1(C)c2cc(N(c3ccc(-c4ccc(C5CC6CCC5C6)cc4)cc3)c3ccccc3-c3ccccc3-c3ccccc3-c3ccccc3)ccc2-c2c(-c3ccccc3)cccc21] 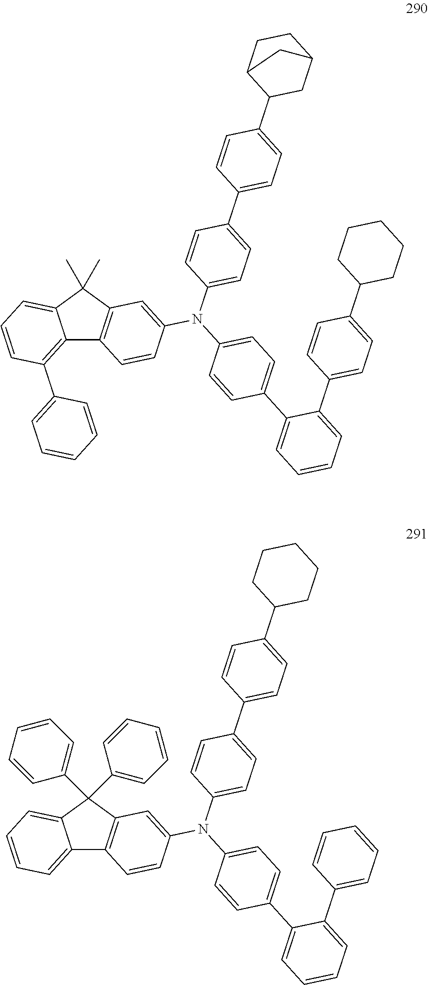 [SMILES: CC1(C)c2cc(N(c3ccc(-c4ccc(C5CC6CCC5C6)cc4)cc3)c3ccc(-c4ccccc4-c4ccc(C5CCCCC5)cc4)cc3)ccc2-c2c(-c3ccccc3)cccc21 ; c1ccc(-c2ccccc2-c2ccc(N(c3ccc(-c4ccc(C5CCCCC5)cc4)cc3)c3ccc4c(c3)C(c3ccccc3)(c3ccccc3)c3ccccc3-4)cc2)cc1] 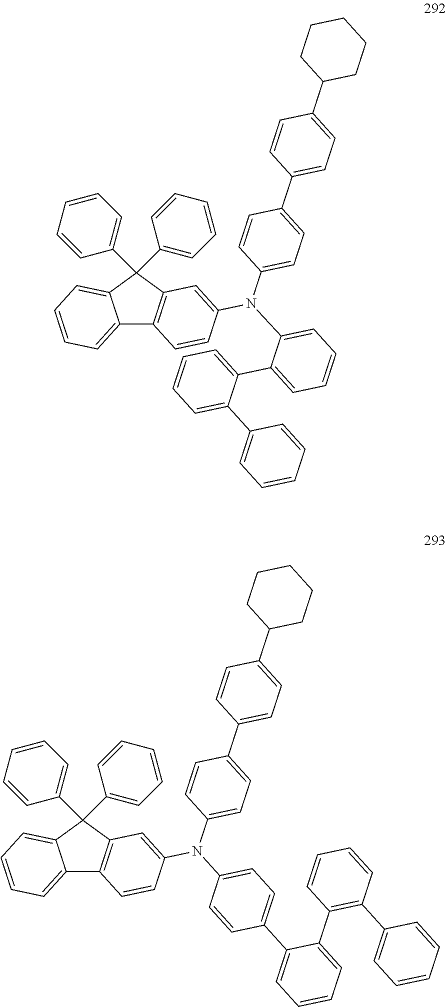 [SMILES: c1ccc(-c2ccccc2-c2ccccc2N(c2ccc(-c3ccc(C4CCCCC4)cc3)cc2)c2ccc3c(c2)C(c2ccccc2)(c2ccccc2)c2ccccc2-3)cc1 ; c1ccc(-c2ccccc2-c2ccccc2-c2ccc(N(c3ccc(-c4ccc(C5CCCCC5)cc4)cc3)c3ccc4c(c3)C(c3ccccc3)(c3ccccc3)c3ccccc3-4)cc2)cc1] 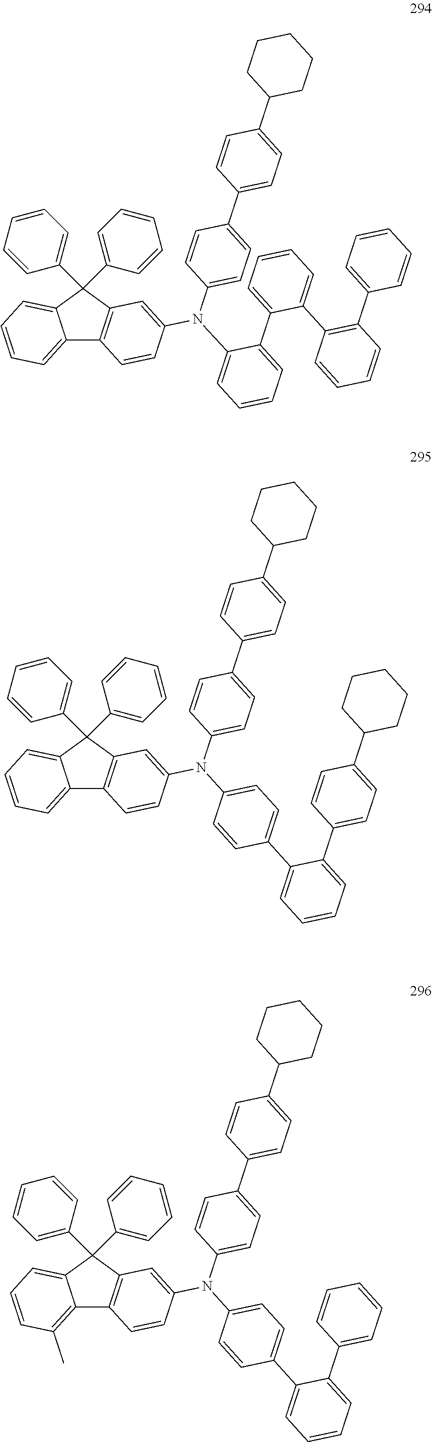 [SMILES: c1ccc(-c2ccccc2-c2ccccc2-c2ccccc2N(c2ccc(-c3ccc(C4CCCCC4)cc3)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccc(C6CCCCC6)cc5)cc4)c4ccc(-c5ccccc5-c5ccc(C6CCCCC6)cc5)cc4)cc32)cc1 ; Cc1cccc2c1-c1ccc(N(c3ccc(-c4ccc(C5CCCCC5)cc4)cc3)c3ccc(-c4ccccc4-c4ccccc4)cc3)cc1C2(c1ccccc1)c1ccccc1] 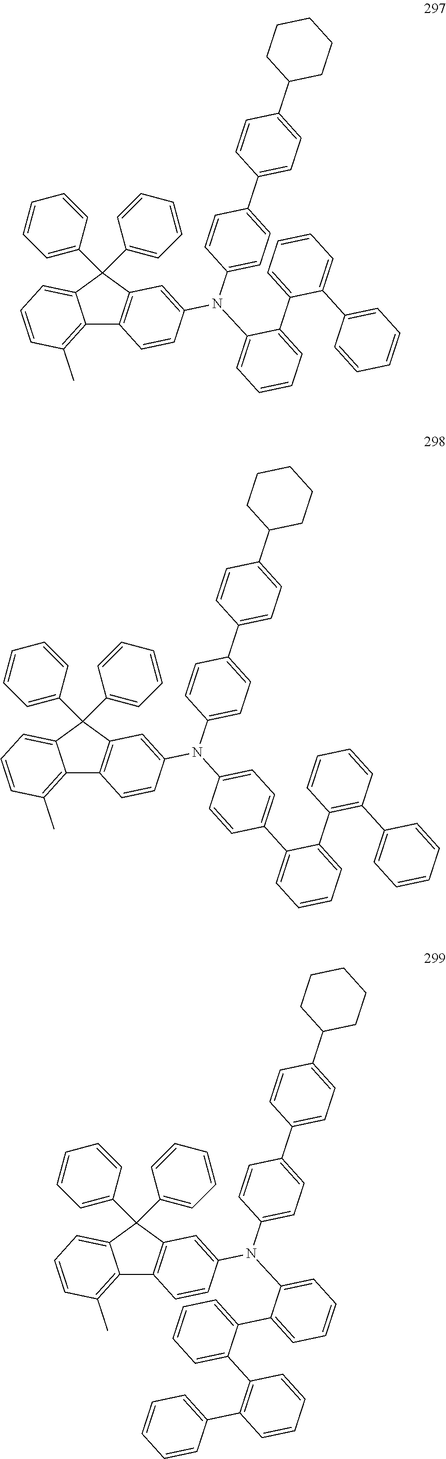 [SMILES: Cc1cccc2c1-c1ccc(N(c3ccc(-c4ccc(C5CCCCC5)cc4)cc3)c3ccccc3-c3ccccc3-c3ccccc3)cc1C2(c1ccccc1)c1ccccc1 ; Cc1cccc2c1-c1ccc(N(c3ccc(-c4ccc(C5CCCCC5)cc4)cc3)c3ccc(-c4ccccc4-c4ccccc4-c4ccccc4)cc3)cc1C2(c1ccccc1)c1ccccc1 ; Cc1cccc2c1-c1ccc(N(c3ccc(-c4ccc(C5CCCCC5)cc4)cc3)c3ccccc3-c3ccccc3-c3ccccc3-c3ccccc3)cc1C2(c1ccccc1)c1ccccc1] 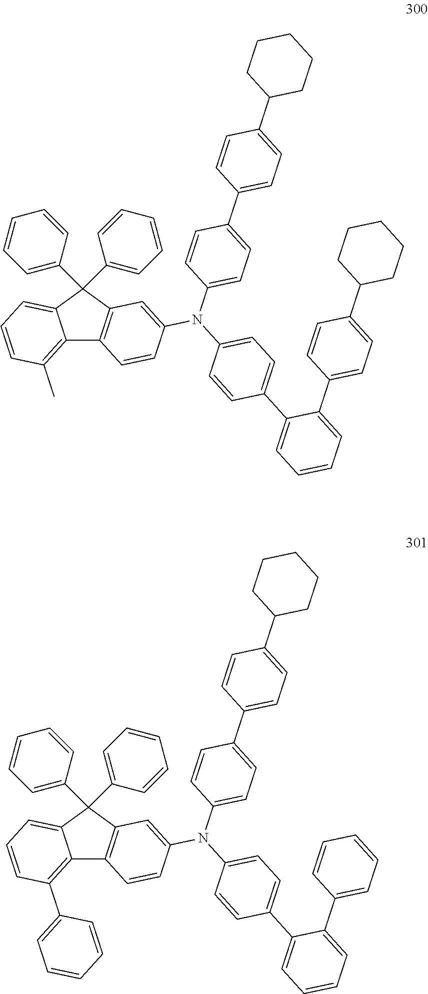 [SMILES: Cc1cccc2c1-c1ccc(N(c3ccc(-c4ccc(C5CCCCC5)cc4)cc3)c3ccc(-c4ccccc4-c4ccc(C5CCCCC5)cc4)cc3)cc1C2(c1ccccc1)c1ccccc1 ; c1ccc(-c2ccccc2-c2ccc(N(c3ccc(-c4ccc(C5CCCCC5)cc4)cc3)c3ccc4c(c3)C(c3ccccc3)(c3ccccc3)c3cccc(-c5ccccc5)c3-4)cc2)cc1] 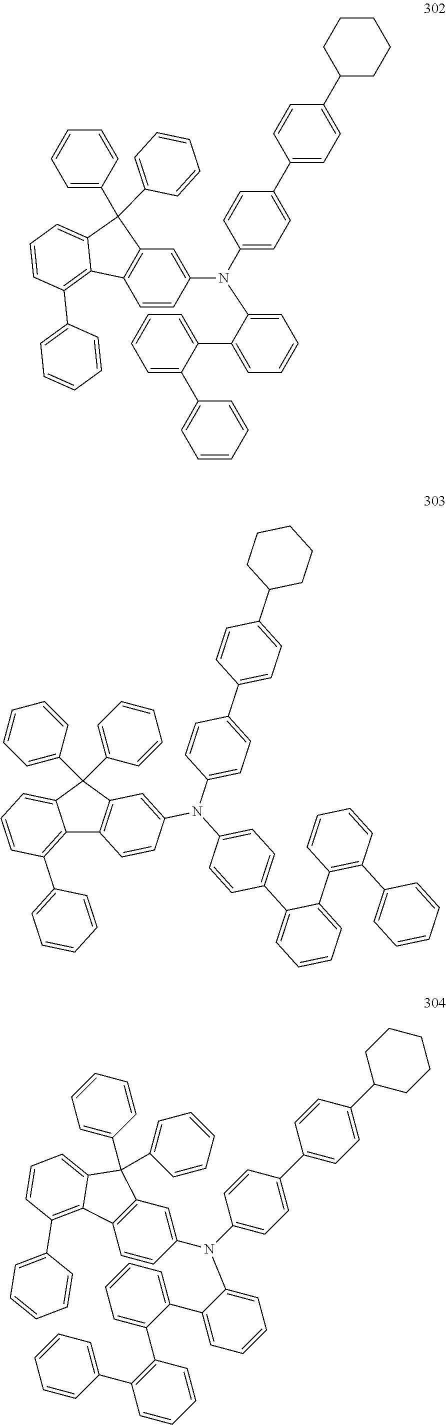 [SMILES: c1ccc(-c2ccccc2-c2ccccc2N(c2ccc(-c3ccc(C4CCCCC4)cc3)cc2)c2ccc3c(c2)C(c2ccccc2)(c2ccccc2)c2cccc(-c4ccccc4)c2-3)cc1 ; c1ccc(-c2ccccc2-c2ccccc2-c2ccc(N(c3ccc(-c4ccc(C5CCCCC5)cc4)cc3)c3ccc4c(c3)C(c3ccccc3)(c3ccccc3)c3cccc(-c5ccccc5)c3-4)cc2)cc1 ; c1ccc(-c2ccccc2-c2ccccc2-c2ccccc2N(c2ccc(-c3ccc(C4CCCCC4)cc3)cc2)c2ccc3c(c2)C(c2ccccc2)(c2ccccc2)c2cccc(-c4ccccc4)c2-3)cc1] 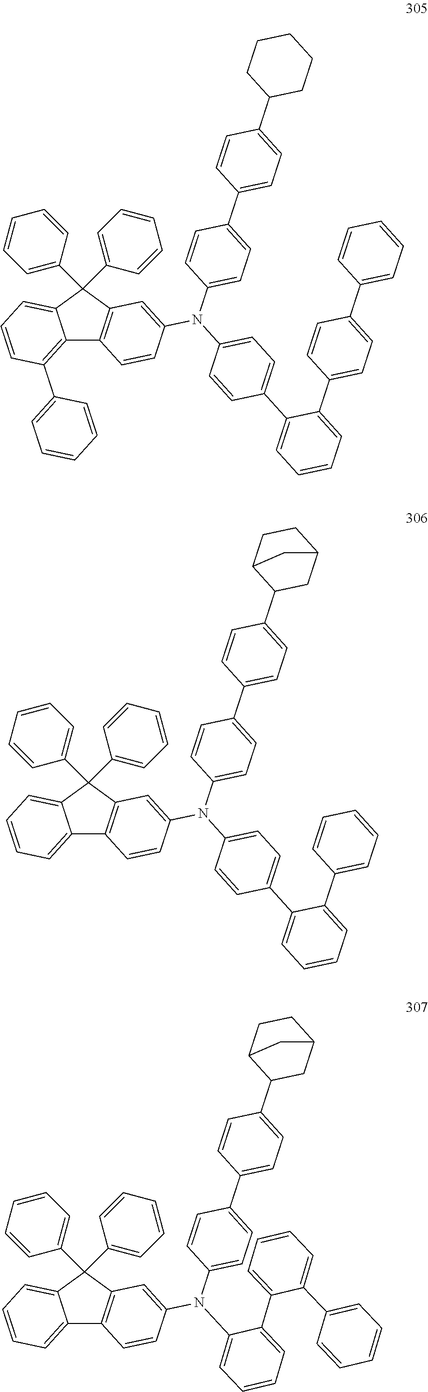 [SMILES: c1ccc(-c2ccc(-c3ccccc3-c3ccc(N(c4ccc(-c5ccc(C6CCCCC6)cc5)cc4)c4ccc5c(c4)C(c4ccccc4)(c4ccccc4)c4cccc(-c6ccccc6)c4-5)cc3)cc2)cc1 ; c1ccc(-c2ccccc2-c2ccc(N(c3ccc(-c4ccc(C5CC6CCC5C6)cc4)cc3)c3ccc4c(c3)C(c3ccccc3)(c3ccccc3)c3ccccc3-4)cc2)cc1 ; c1ccc(-c2ccccc2-c2ccccc2N(c2ccc(-c3ccc(C4CC5CCC4C5)cc3)cc2)c2ccc3c(c2)C(c2ccccc2)(c2ccccc2)c2ccccc2-3)cc1] 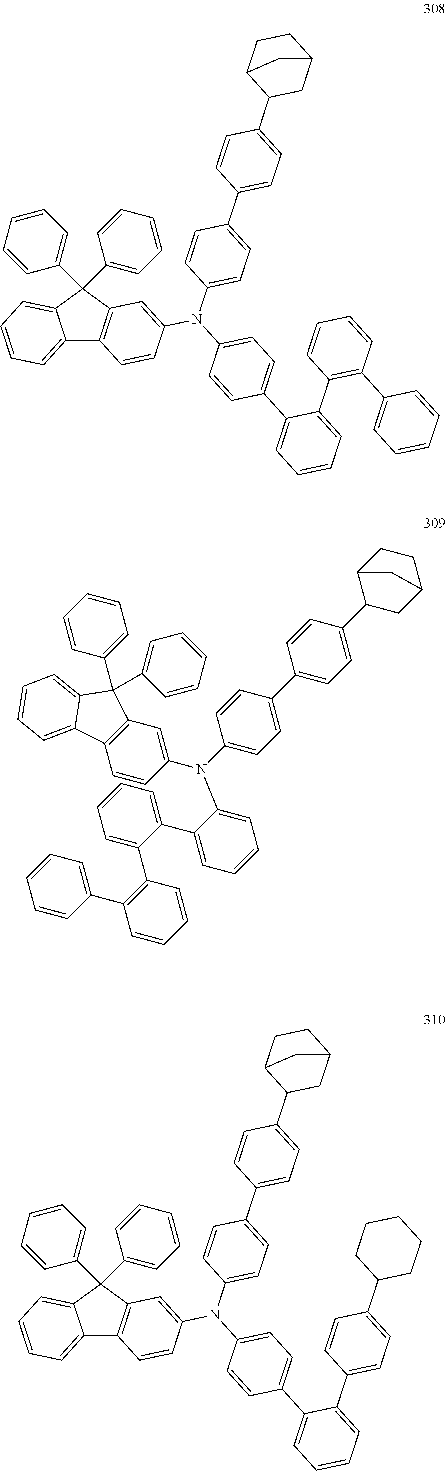 [SMILES: c1ccc(-c2ccccc2-c2ccccc2-c2ccc(N(c3ccc(-c4ccc(C5CC6CCC5C6)cc4)cc3)c3ccc4c(c3)C(c3ccccc3)(c3ccccc3)c3ccccc3-4)cc2)cc1 ; c1ccc(-c2ccccc2-c2ccccc2-c2ccccc2N(c2ccc(-c3ccc(C4CC5CCC4C5)cc3)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5ccc(C6CC7CCC6C7)cc5)cc4)c4ccc(-c5ccccc5-c5ccc(C6CCCCC6)cc5)cc4)cc32)cc1] 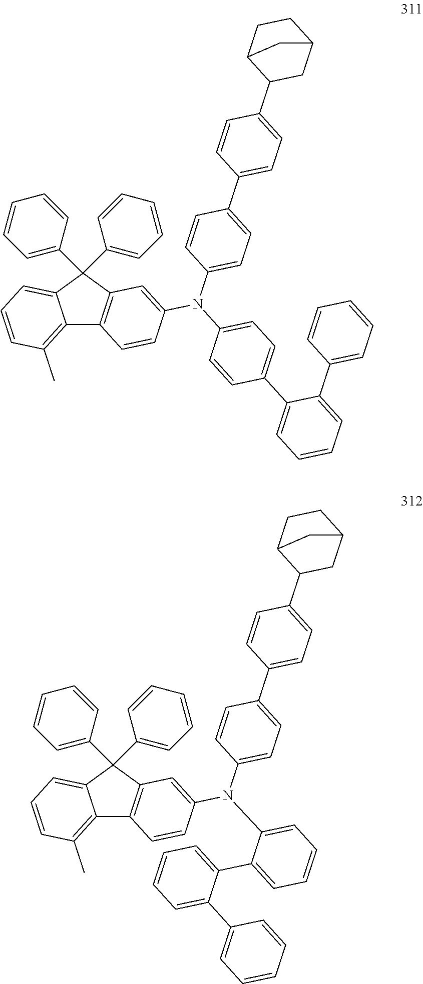 [SMILES: Cc1cccc2c1-c1ccc(N(c3ccc(-c4ccc(C5CC6CCC5C6)cc4)cc3)c3ccc(-c4ccccc4-c4ccccc4)cc3)cc1C2(c1ccccc1)c1ccccc1 ; Cc1cccc2c1-c1ccc(N(c3ccc(-c4ccc(C5CC6CCC5C6)cc4)cc3)c3ccccc3-c3ccccc3-c3ccccc3)cc1C2(c1ccccc1)c1ccccc1] 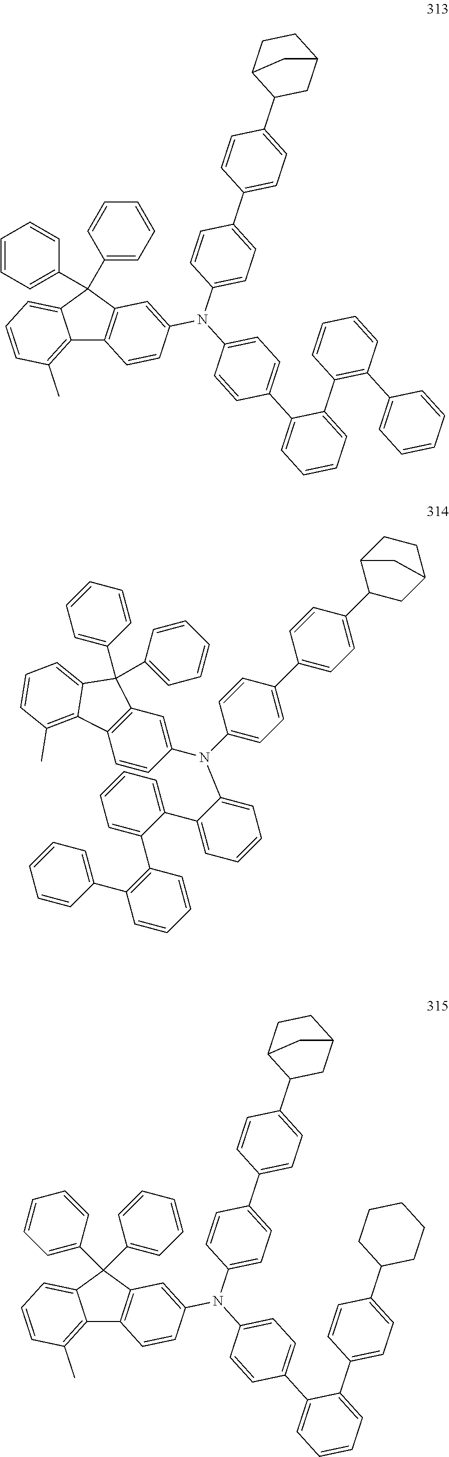 [SMILES: Cc1cccc2c1-c1ccc(N(c3ccc(-c4ccc(C5CC6CCC5C6)cc4)cc3)c3ccc(-c4ccccc4-c4ccccc4-c4ccccc4)cc3)cc1C2(c1ccccc1)c1ccccc1 ; Cc1cccc2c1-c1ccc(N(c3ccc(-c4ccc(C5CC6CCC5C6)cc4)cc3)c3ccccc3-c3ccccc3-c3ccccc3-c3ccccc3)cc1C2(c1ccccc1)c1ccccc1 ; Cc1cccc2c1-c1ccc(N(c3ccc(-c4ccc(C5CC6CCC5C6)cc4)cc3)c3ccc(-c4ccccc4-c4ccc(C5CCCCC5)cc4)cc3)cc1C2(c1ccccc1)c1ccccc1] 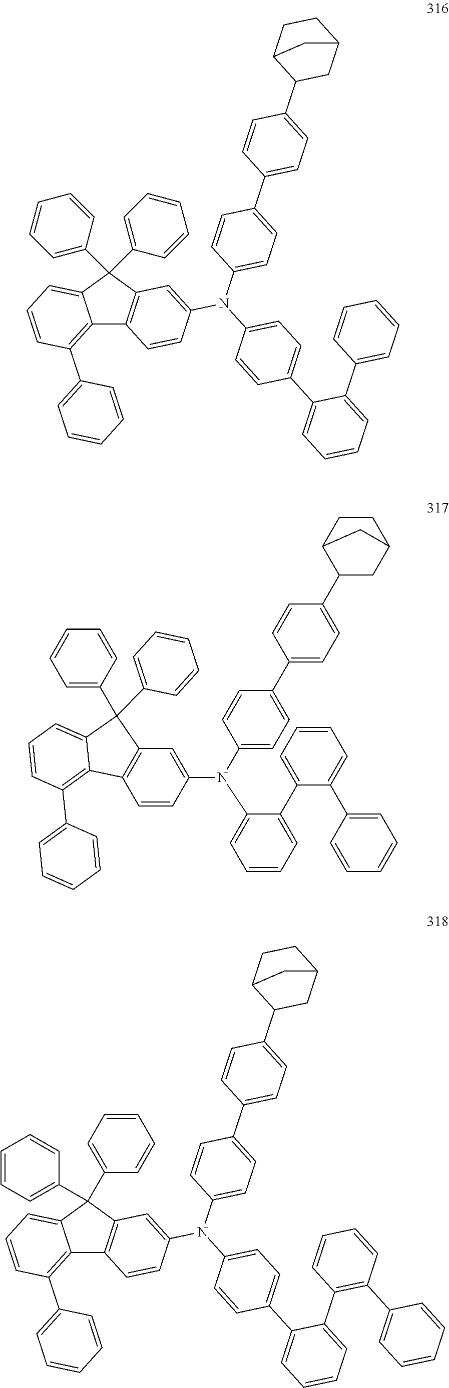 [SMILES: c1ccc(-c2ccccc2-c2ccc(N(c3ccc(-c4ccc(C5CC6CCC5C6)cc4)cc3)c3ccc4c(c3)C(c3ccccc3)(c3ccccc3)c3cccc(-c5ccccc5)c3-4)cc2)cc1 ; c1ccc(-c2ccccc2-c2ccccc2N(c2ccc(-c3ccc(C4CC5CCC4C5)cc3)cc2)c2ccc3c(c2)C(c2ccccc2)(c2ccccc2)c2cccc(-c4ccccc4)c2-3)cc1 ; c1ccc(-c2ccccc2-c2ccccc2-c2ccc(N(c3ccc(-c4ccc(C5CC6CCC5C6)cc4)cc3)c3ccc4c(c3)C(c3ccccc3)(c3ccccc3)c3cccc(-c5ccccc5)c3-4)cc2)cc1] 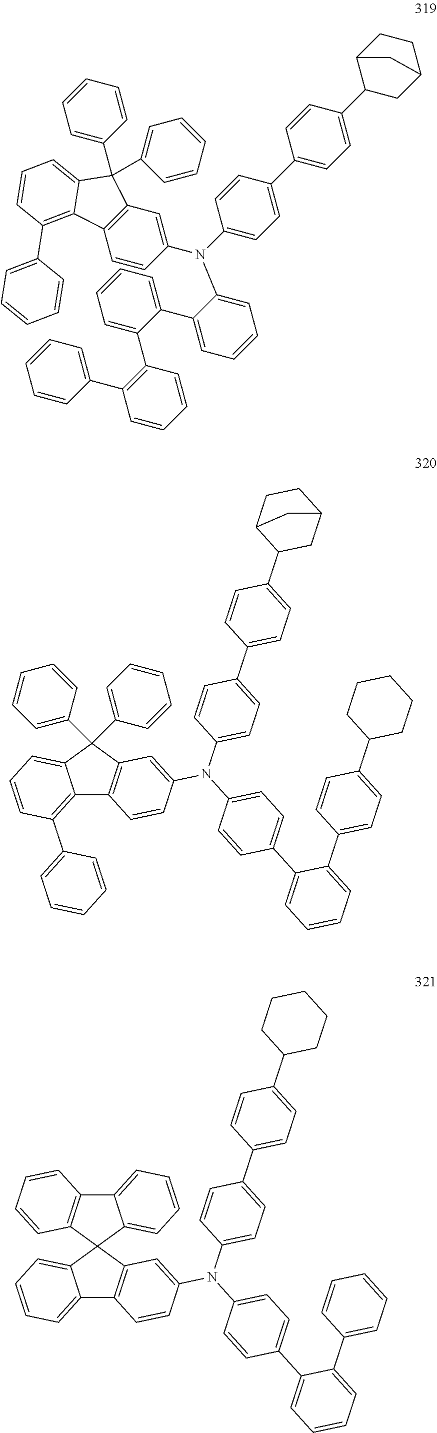 [SMILES: c1ccc(-c2ccccc2-c2ccccc2-c2ccccc2N(c2ccc(-c3ccc(C4CC5CCC4C5)cc3)cc2)c2ccc3c(c2)C(c2ccccc2)(c2ccccc2)c2cccc(-c4ccccc4)c2-3)cc1 ; c1ccc(-c2cccc3c2-c2ccc(N(c4ccc(-c5ccc(C6CC7CCC6C7)cc5)cc4)c4ccc(-c5ccccc5-c5ccc(C6CCCCC6)cc5)cc4)cc2C3(c2ccccc2)c2ccccc2)cc1 ; c1ccc(-c2ccccc2-c2ccc(N(c3ccc(-c4ccc(C5CCCCC5)cc4)cc3)c3ccc4c(c3)C3(c5ccccc5-c5ccccc53)c3ccccc3-4)cc2)cc1] 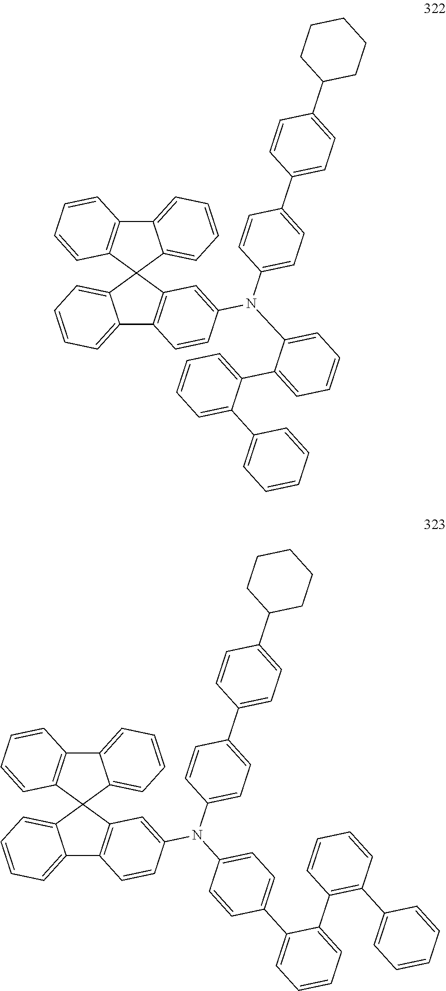 [SMILES: c1ccc(-c2ccccc2-c2ccccc2N(c2ccc(-c3ccc(C4CCCCC4)cc3)cc2)c2ccc3c(c2)C2(c4ccccc4-c4ccccc42)c2ccccc2-3)cc1 ; c1ccc(-c2ccccc2-c2ccccc2-c2ccc(N(c3ccc(-c4ccc(C5CCCCC5)cc4)cc3)c3ccc4c(c3)C3(c5ccccc5-c5ccccc53)c3ccccc3-4)cc2)cc1] 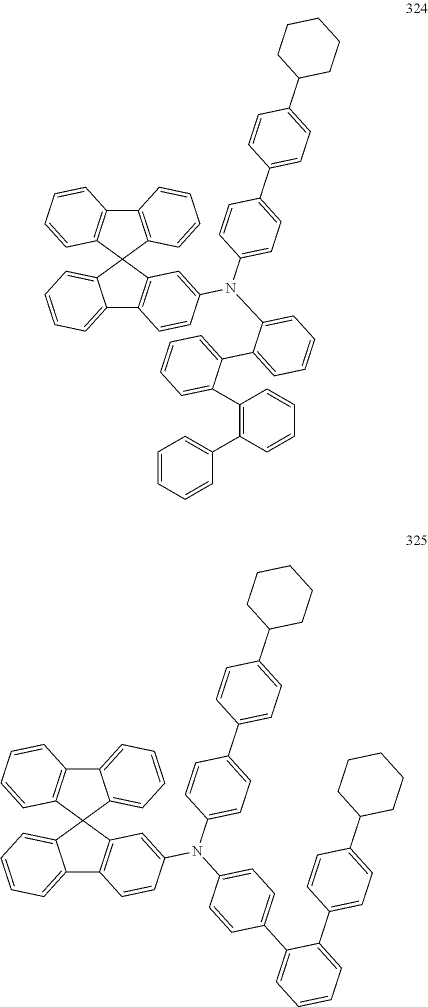 [SMILES: c1ccc(-c2ccccc2-c2ccccc2-c2ccccc2N(c2ccc(-c3ccc(C4CCCCC4)cc3)cc2)c2ccc3c(c2)C2(c4ccccc4-c4ccccc42)c2ccccc2-3)cc1 ; c1ccc(-c2ccc(N(c3ccc(-c4ccc(C5CCCCC5)cc4)cc3)c3ccc4c(c3)C3(c5ccccc5-c5ccccc53)c3ccccc3-4)cc2)c(-c2ccc(C3CCCCC3)cc2)c1] 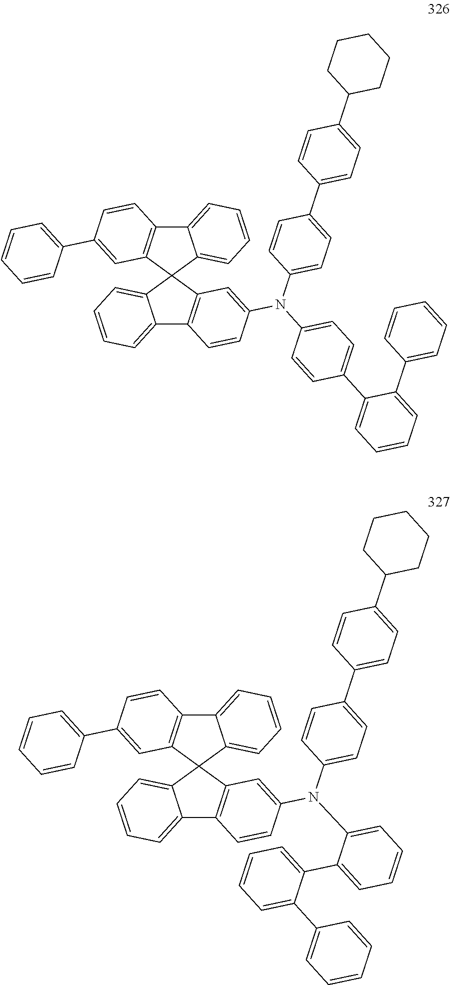 [SMILES: c1ccc(-c2ccc3c(c2)C2(c4ccccc4-3)c3ccccc3-c3ccc(N(c4ccc(-c5ccc(C6CCCCC6)cc5)cc4)c4ccc(-c5ccccc5-c5ccccc5)cc4)cc32)cc1 ; c1ccc(-c2ccc3c(c2)C2(c4ccccc4-3)c3ccccc3-c3ccc(N(c4ccc(-c5ccc(C6CCCCC6)cc5)cc4)c4ccccc4-c4ccccc4-c4ccccc4)cc32)cc1] 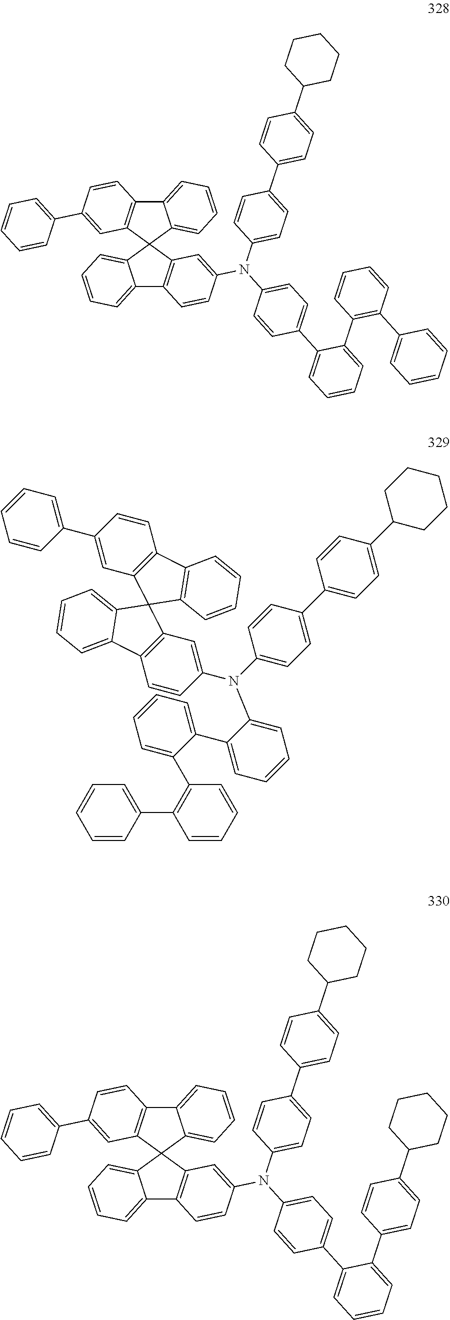 [SMILES: c1ccc(-c2ccc3c(c2)C2(c4ccccc4-3)c3ccccc3-c3ccc(N(c4ccc(-c5ccc(C6CCCCC6)cc5)cc4)c4ccc(-c5ccccc5-c5ccccc5-c5ccccc5)cc4)cc32)cc1 ; c1ccc(-c2ccc3c(c2)C2(c4ccccc4-3)c3ccccc3-c3ccc(N(c4ccc(-c5ccc(C6CCCCC6)cc5)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccc(C6CCCCC6)cc5)cc4)c4ccc(-c5ccccc5-c5ccc(C6CCCCC6)cc5)cc4)cc32)cc1] 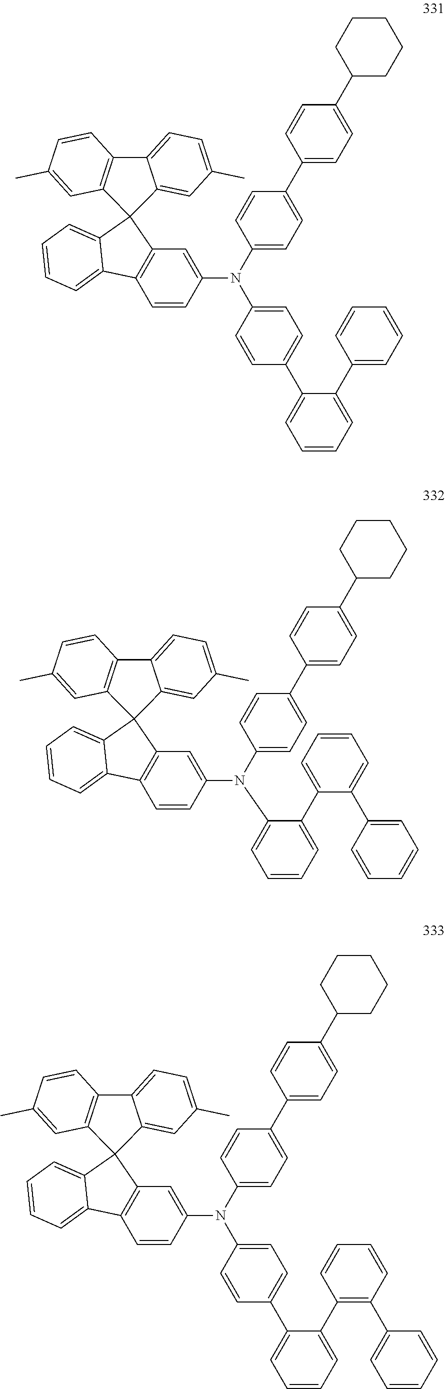 [SMILES: Cc1ccc2c(c1)C1(c3ccccc3-c3ccc(N(c4ccc(-c5ccc(C6CCCCC6)cc5)cc4)c4ccc(-c5ccccc5-c5ccccc5)cc4)cc31)c1cc(C)ccc1-2 ; Cc1ccc2c(c1)C1(c3ccccc3-c3ccc(N(c4ccc(-c5ccc(C6CCCCC6)cc5)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4ccc(-c5ccccc5-c5ccccc5-c5ccccc5)cc4)cc31)c1cc(C)ccc1-2] 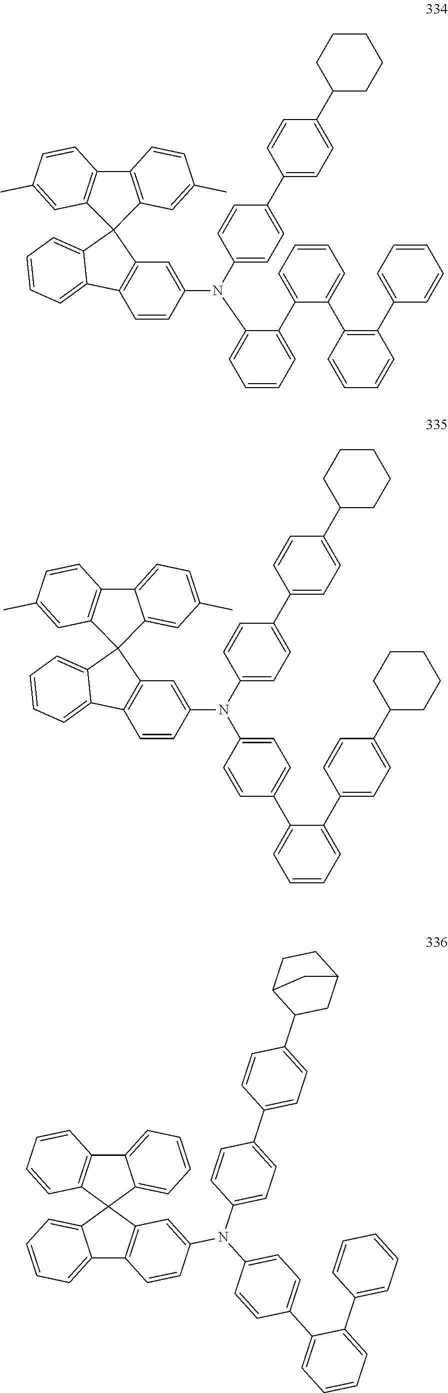 [SMILES: Cc1ccc2c(c1)C1(c3ccccc3-c3ccc(N(c4ccc(-c5ccc(C6CCCCC6)cc5)cc4)c4ccccc4-c4ccccc4-c4ccccc4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4ccc(-c5ccccc5-c5ccc(C6CCCCC6)cc5)cc4)cc31)c1cc(C)ccc1-2 ; c1ccc(-c2ccccc2-c2ccc(N(c3ccc(-c4ccc(C5CC6CCC5C6)cc4)cc3)c3ccc4c(c3)C3(c5ccccc5-c5ccccc53)c3ccccc3-4)cc2)cc1] 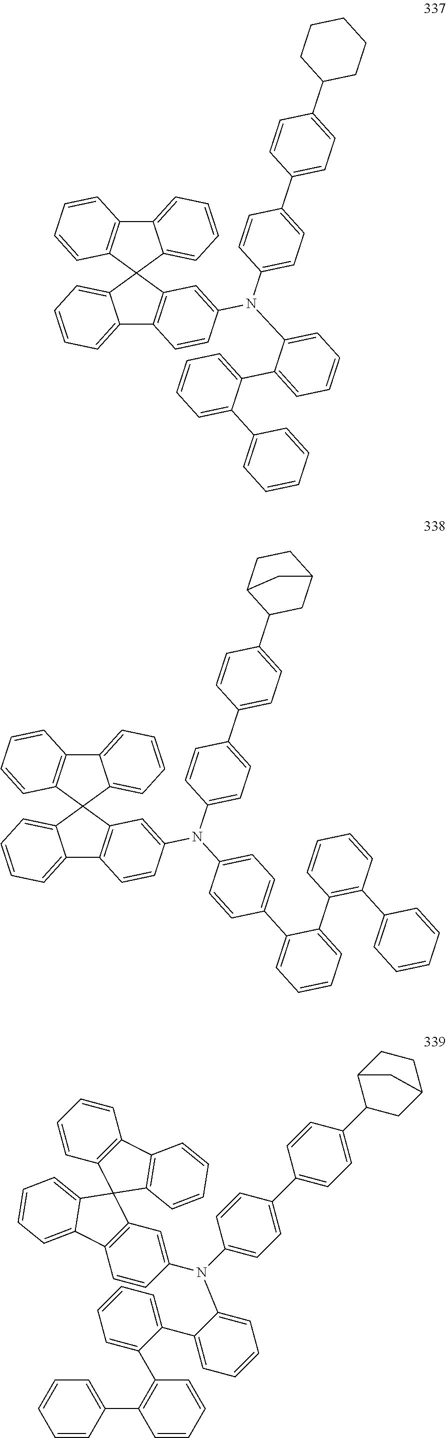 [SMILES: c1ccc(-c2ccccc2-c2ccccc2N(c2ccc(-c3ccc(C4CCCCC4)cc3)cc2)c2ccc3c(c2)C2(c4ccccc4-c4ccccc42)c2ccccc2-3)cc1 ; c1ccc(-c2ccccc2-c2ccccc2-c2ccc(N(c3ccc(-c4ccc(C5CC6CCC5C6)cc4)cc3)c3ccc4c(c3)C3(c5ccccc5-c5ccccc53)c3ccccc3-4)cc2)cc1 ; c1ccc(-c2ccccc2-c2ccccc2-c2ccccc2N(c2ccc(-c3ccc(C4CC5CCC4C5)cc3)cc2)c2ccc3c(c2)C2(c4ccccc4-c4ccccc42)c2ccccc2-3)cc1] 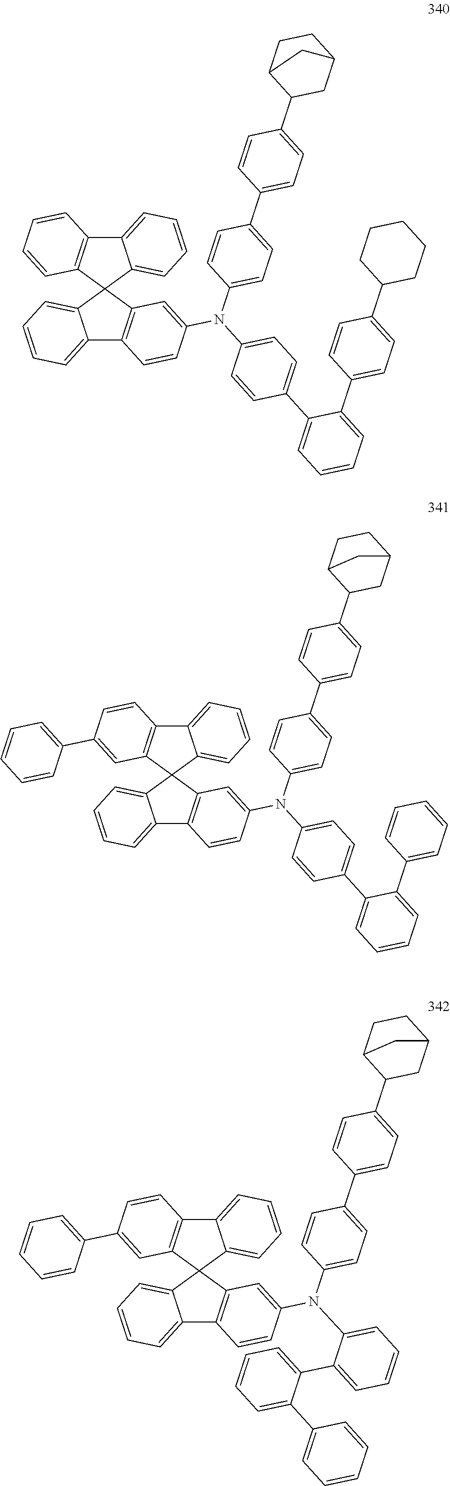 [SMILES: c1ccc(-c2ccc(N(c3ccc(-c4ccc(C5CC6CCC5C6)cc4)cc3)c3ccc4c(c3)C3(c5ccccc5-c5ccccc53)c3ccccc3-4)cc2)c(-c2ccc(C3CCCCC3)cc2)c1 ; c1ccc(-c2ccc3c(c2)C2(c4ccccc4-3)c3ccccc3-c3ccc(N(c4ccc(-c5ccc(C6CC7CCC6C7)cc5)cc4)c4ccc(-c5ccccc5-c5ccccc5)cc4)cc32)cc1 ; c1ccc(-c2ccc3c(c2)C2(c4ccccc4-3)c3ccccc3-c3ccc(N(c4ccc(-c5ccc(C6CC7CCC6C7)cc5)cc4)c4ccccc4-c4ccccc4-c4ccccc4)cc32)cc1] 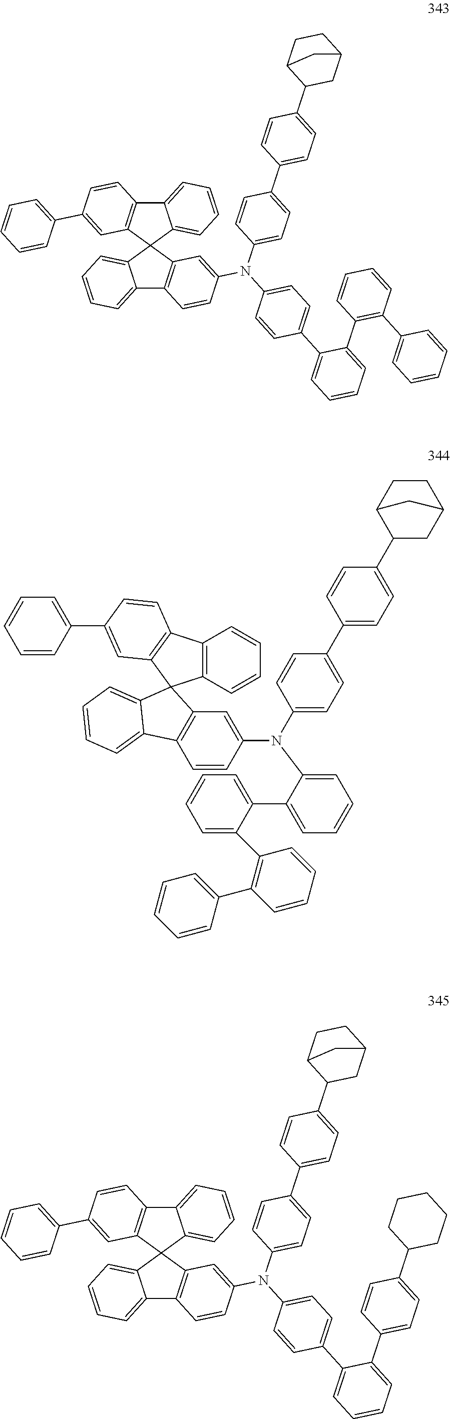 [SMILES: c1ccc(-c2ccc3c(c2)C2(c4ccccc4-3)c3ccccc3-c3ccc(N(c4ccc(-c5ccc(C6CC7CCC6C7)cc5)cc4)c4ccc(-c5ccccc5-c5ccccc5-c5ccccc5)cc4)cc32)cc1 ; c1ccc(-c2ccc3c(c2)C2(c4ccccc4-3)c3ccccc3-c3ccc(N(c4ccc(-c5ccc(C6CC7CCC6C7)cc5)cc4)c4ccccc4-c4ccccc4-c4ccccc4-c4ccccc4)cc32)cc1 ; c1ccc(-c2ccc3c(c2)C2(c4ccccc4-3)c3ccccc3-c3ccc(N(c4ccc(-c5ccc(C6CC7CCC6C7)cc5)cc4)c4ccc(-c5ccccc5-c5ccc(C6CCCCC6)cc5)cc4)cc32)cc1] 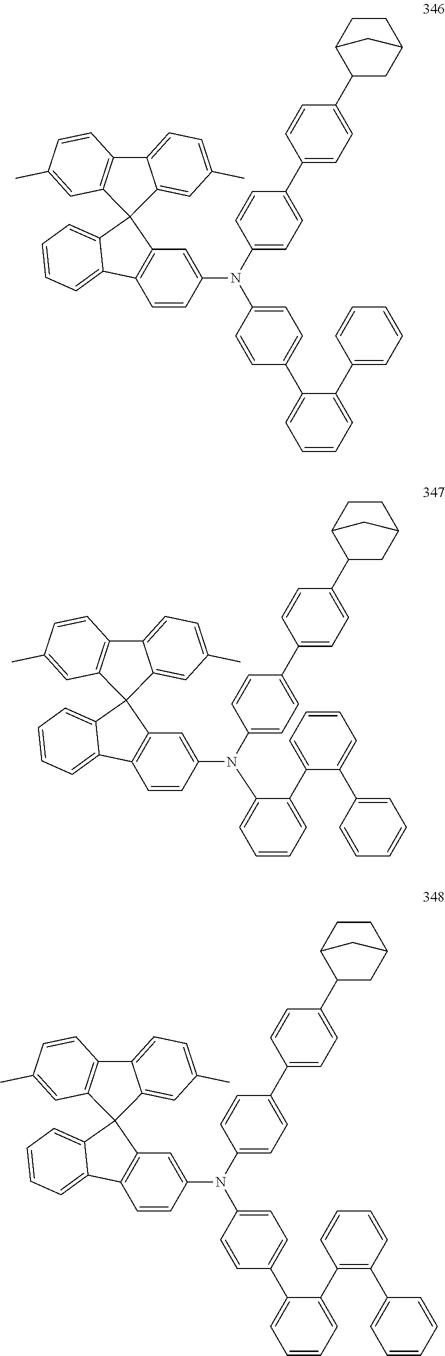 [SMILES: Cc1ccc2c(c1)C1(c3ccccc3-c3ccc(N(c4ccc(-c5ccc(C6CC7CCC6C7)cc5)cc4)c4ccc(-c5ccccc5-c5ccccc5)cc4)cc31)c1cc(C)ccc1-2 ; Cc1ccc2c(c1)C1(c3ccccc3-c3ccc(N(c4ccc(-c5ccc(C6CC7CCC6C7)cc5)cc4)c4ccccc4-c4ccccc4-c4ccccc4)cc31)c1cc(C)ccc1-2 ; Cc1ccc2c(c1)C1(c3ccccc3-c3ccc(N(c4ccc(-c5ccc(C6CC7CCC6C7)cc5)cc4)c4ccc(-c5ccccc5-c5ccccc5-c5ccccc5)cc4)cc31)c1cc(C)ccc1-2] 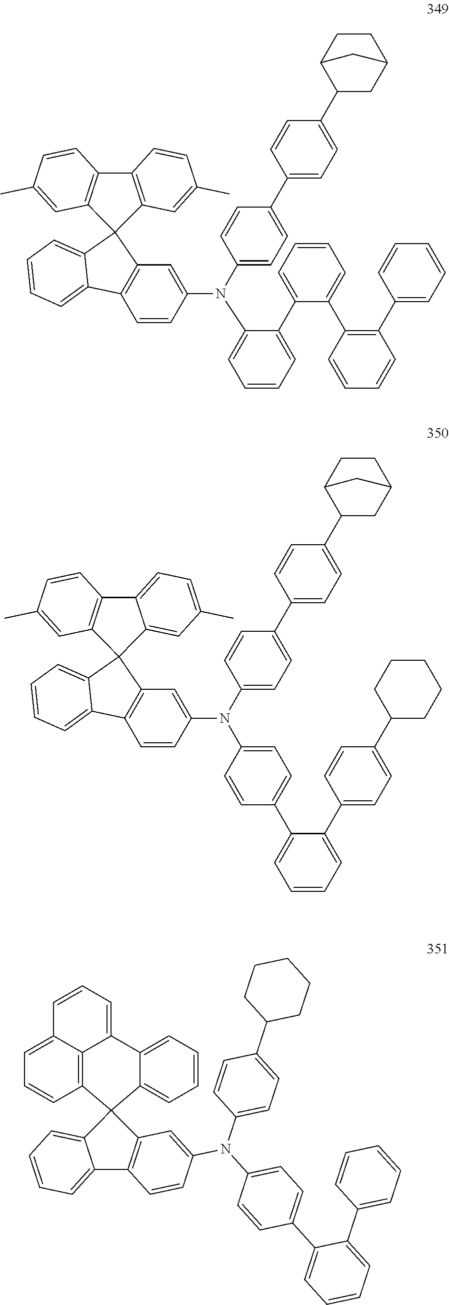 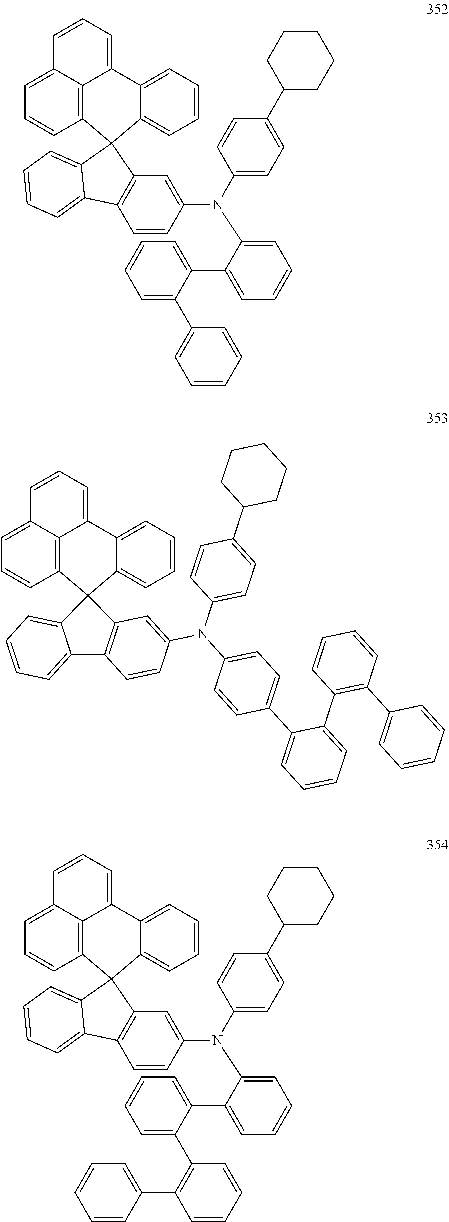 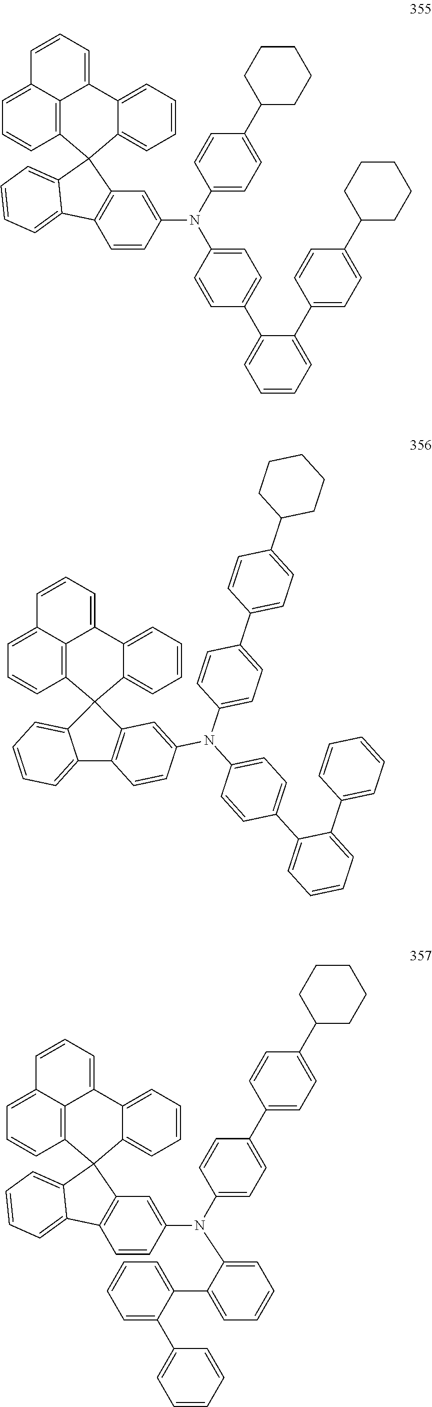 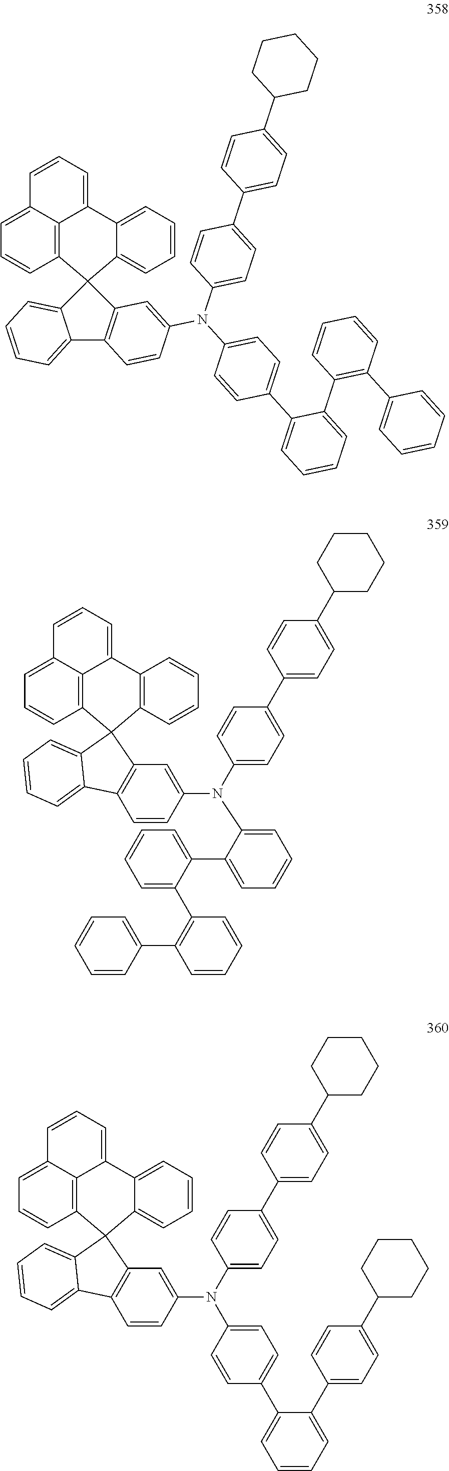 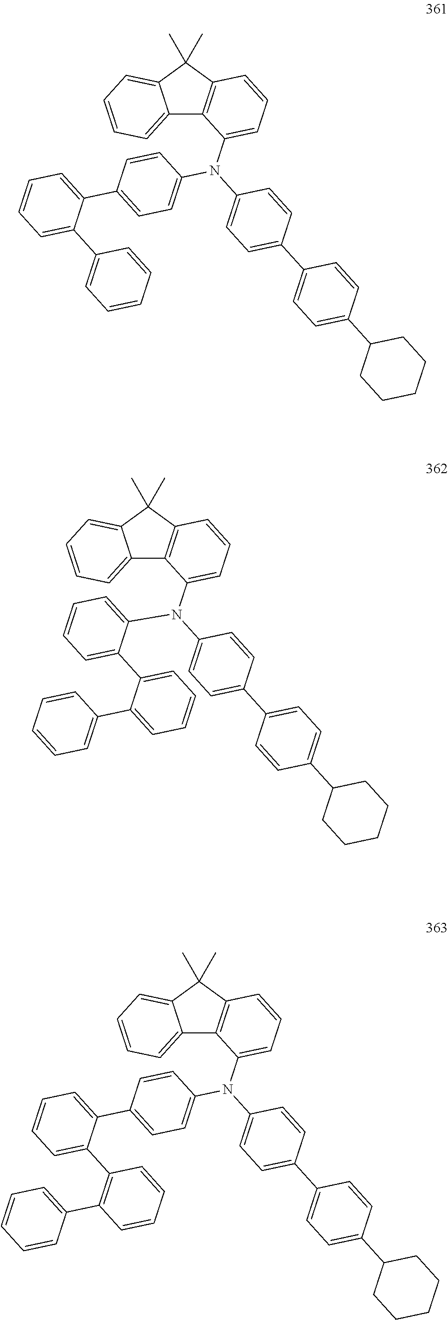 [SMILES: CC1(C)c2ccccc2-c2c(N(c3ccc(-c4ccc(C5CCCCC5)cc4)cc3)c3ccc(-c4ccccc4-c4ccccc4)cc3)cccc21 ; CC1(C)c2ccccc2-c2c(N(c3ccc(-c4ccc(C5CCCCC5)cc4)cc3)c3ccccc3-c3ccccc3-c3ccccc3)cccc21 ; CC1(C)c2ccccc2-c2c(N(c3ccc(-c4ccc(C5CCCCC5)cc4)cc3)c3ccc(-c4ccccc4-c4ccccc4-c4ccccc4)cc3)cccc21] 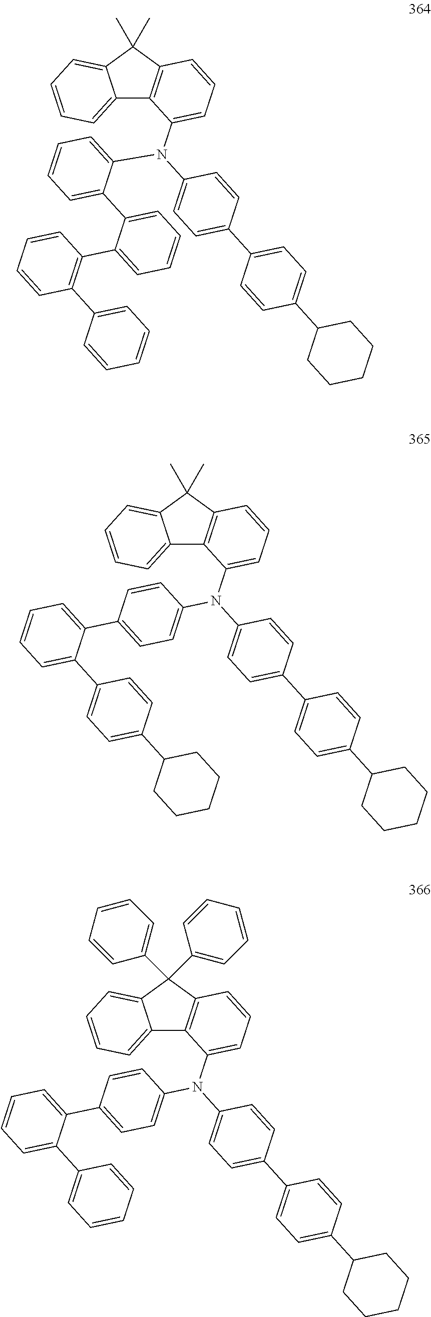 [SMILES: CC1(C)c2ccccc2-c2c(N(c3ccc(-c4ccc(C5CCCCC5)cc4)cc3)c3ccccc3-c3ccccc3-c3ccccc3-c3ccccc3)cccc21 ; CC1(C)c2ccccc2-c2c(N(c3ccc(-c4ccc(C5CCCCC5)cc4)cc3)c3ccc(-c4ccccc4-c4ccc(C5CCCCC5)cc4)cc3)cccc21 ; c1ccc(-c2ccccc2-c2ccc(N(c3ccc(-c4ccc(C5CCCCC5)cc4)cc3)c3cccc4c3-c3ccccc3C4(c3ccccc3)c3ccccc3)cc2)cc1] 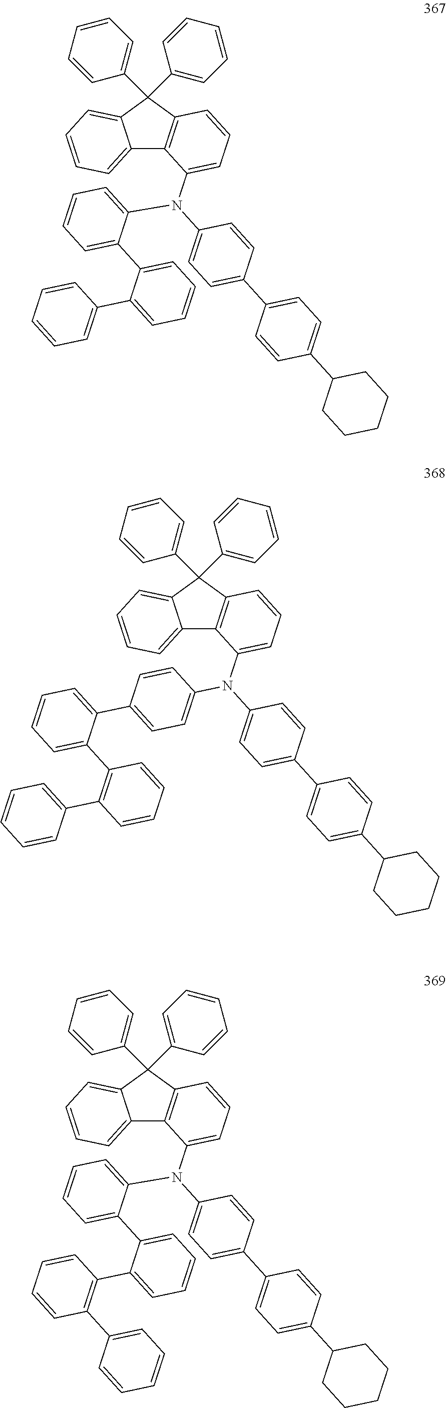 [SMILES: c1ccc(-c2ccccc2-c2ccccc2N(c2ccc(-c3ccc(C4CCCCC4)cc3)cc2)c2cccc3c2-c2ccccc2C3(c2ccccc2)c2ccccc2)cc1 ; c1ccc(-c2ccccc2-c2ccccc2-c2ccc(N(c3ccc(-c4ccc(C5CCCCC5)cc4)cc3)c3cccc4c3-c3ccccc3C4(c3ccccc3)c3ccccc3)cc2)cc1 ; c1ccc(-c2ccccc2-c2ccccc2-c2ccccc2N(c2ccc(-c3ccc(C4CCCCC4)cc3)cc2)c2cccc3c2-c2ccccc2C3(c2ccccc2)c2ccccc2)cc1] 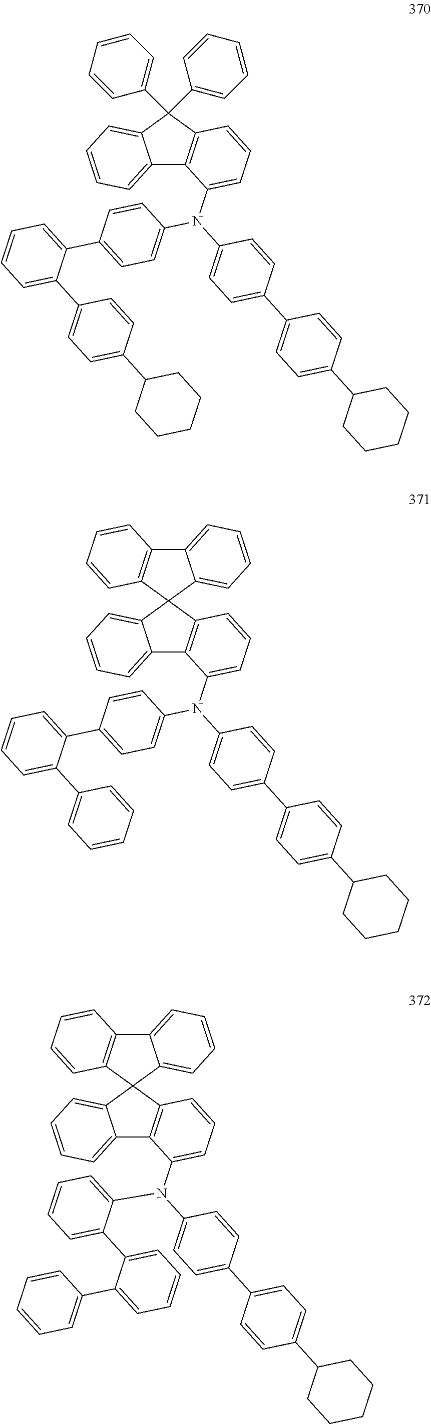 [SMILES: c1ccc(C2(c3ccccc3)c3ccccc3-c3c(N(c4ccc(-c5ccc(C6CCCCC6)cc5)cc4)c4ccc(-c5ccccc5-c5ccc(C6CCCCC6)cc5)cc4)cccc32)cc1 ; c1ccc(-c2ccccc2-c2ccc(N(c3ccc(-c4ccc(C5CCCCC5)cc4)cc3)c3cccc4c3-c3ccccc3C43c4ccccc4-c4ccccc43)cc2)cc1 ; c1ccc(-c2ccccc2-c2ccccc2N(c2ccc(-c3ccc(C4CCCCC4)cc3)cc2)c2cccc3c2-c2ccccc2C32c3ccccc3-c3ccccc32)cc1] 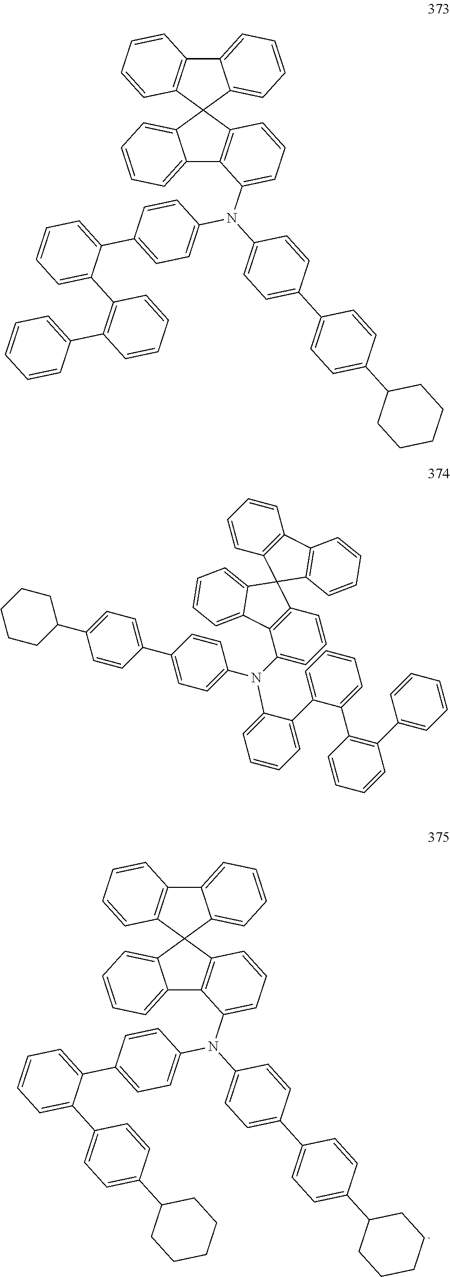 [SMILES: c1ccc(-c2ccccc2-c2ccccc2-c2ccc(N(c3ccc(-c4ccc(C5CCCCC5)cc4)cc3)c3cccc4c3-c3ccccc3C43c4ccccc4-c4ccccc43)cc2)cc1 ; c1ccc(-c2ccccc2-c2ccccc2-c2ccccc2N(c2ccc(-c3ccc(C4CCCCC4)cc3)cc2)c2cccc3c2-c2ccccc2C32c3ccccc3-c3ccccc32)cc1 ; c1ccc(-c2ccc(N(c3ccc(-c4ccc(C5CCCCC5)cc4)cc3)c3cccc4c3-c3ccccc3C43c4ccccc4-c4ccccc43)cc2)c(-c2ccc(C3CCCCC3)cc2)c1]
